(19) United States
(12) Patent Application Publication
(10) Pub. No.: US 2024/0368133 A1
(43) Pub. Date: Nov. 7, 2024

(54) Compounds for the Treatment of Pain, in Particular, Neuropathic Pain, and/or Other Diseases or Disorders that are Associated with AT2R and/or AT2R Mediated Signaling

(71) Applicant: Confo Therapeutics N.V., Gent-Zwijnaarde (BE)

(72) Inventors: Ann DE BLIECK, Sinaai (BE); Christel Jeanne Marie MENET, Brussels (BE)

(21) Appl. No.: 18/293,160

(22) PCT Filed: Jul. 28, 2022
(86) PCT No.: PCT/EP2022/071231

§ 371 Date: Jan. 29, 2024

Related U.S. Application Data
(60) Provisional application No. 63/227,555, filed on Jul. 30, 2021.
(60) Provisional application No. 63/227,552, filed on Jul. 30, 2021.

(51) Int. Cl. C07D 403/14; A61K 31/496; A61K 31/501; A61K 31/506; A61K 31/517; A61P 25/04; C07D 401/14; C07D 409/14; C07D 413/14; C07D 417/14

(52) U.S. Cl. CPC C07D 403/14; A61K 31/496; A61K 31/501; A61K 31/506; A61K 31/517; A61P 25/04; C07D 401/14; C07D 409/14; C07D 413/14; C07D 417/14

(57) ABSTRACT

The present invention relates to compounds that can be used for the prevention, treatment and/or management of pain, in particular chronic pain, such as neuropathic pain, and/or other diseases or disorders that are associated with AT2R and/or AT2R-mediated signaling.

### FIELD

The present invention relates to compounds that can be used for the prevention, treatment and/or management of pain, in particular chronic pain, such as neuropathic pain, and/or other diseases or disorders that are associated with AT2R and/or AT2R-mediated signaling (as further described herein).

### BACKGROUND

Pain can occur in many forms and can have various causes and underlying pathophysiological mechanisms. Pain can be spontaneous, chronic or acute, and can for example be caused by physical damage or potential damage to the body (so-called “nociceptive pain”) or by damage to and/or disease of the somatosensory nervous system (so-called “neuropathic pain”), such as pain caused by damage to or disease of the peripheral nerves (i.e. the nerves beyond the brain and the spinal cord), which is also referred to as “peripheral neuropathy” or “neuropathy” for short.

Pain states can also take the form of hypersensitivity to pain, for example in case of so-called “inflammatory pain”, which term is generally used to describe the spontaneous hypersensitivity to pain that occurs in response to tissue damage and inflammation (e.g. post-operative pain, trauma, arthritis). Persistent pain states are often associated with the development of hyperalgesia (an increase in pain evoked by noxious stimuli and also a lowered threshold for pain) and/or allodynia (an increase in sensitivity to previously non-noxious levels of stimulation), although the term hyperalgesia has also been used in the literature to collectively refer to both hyperalgesia and allodynia (see for example Guindon and Hohmann, British Journal of Pharmacology (2008) 153, 319-334).

The prevention, treatment and/or management of chronic and severe pain in patients has been described, in the words of Guindon and Hohmann (supra), as “the burden of clinicians”.

Various animal models have been developed to experimentally assess pathophysiological mechanisms underlying distinct clinical pain states induced by tissue injury, inflammation, nerve trauma, chemotherapeutic agents and metabolic challenges, which models also permit pre-clinical evaluation and validation of the therapeutic efficacy of putative analgesics. Reference is for example made to Dubner and Ren, “Assessing transient and persistent pain in animals”, In: Textbook of Pain, 4^{th} edition (Wall and Melzack, Eds.), pp. 359-369, Churchill Livingstone, 1999, 53, 319-334 (also cited in Guindon and Hohmann, supra).

Also, various molecular targets for the prevention, treatment and/or management of pain have been suggested in the art. These include cannabinoid CB2 receptors (see again for example Guindon and Hohmann, supra), NMDA receptors (see for example Parsons, European Journal of Pharmacology, 429 Z2001, 71-78), various ion channels (see for example Dib-Hajj et al., Brain Research Reviews, Volume 60, Issue 1, April 2009, Pages 65-83 and Markman and Dworkin, The Journal of Pain, Volume 7, Issue 1, Supplement, January 2006, Pages S38-S47), sphingosine-1-phosphate receptors (see for example Welch et al., Biochemical Pharmacology, Volume 84, Issue 12, 15 Dec. 2012, Pages 1551-1562) and Monoacylglycerol lipase (MAGL) (see for example WO 2020/112905).

Neuropathic pain is a chronic secondary pain condition caused by damage to and/or disease of the somatosensory nervous system that is generally characterized by hyperalgesia and/or allodynia. Neuropathic pain affects around 7 to 10% of the general population and can have a major impact on quality of life. Reference is for example made to Szok et al., Behav. Neurol., 2019: 8685954; Colloca et al., Nat. Rev. Dis. Primers, 2017 Feb. 16, 3: 17002; Alles and Smith, Pharmacol. Rev. 70: 315-347, April 2018; and Cavalli et al., Int. J. Immunopathol. Pharmacol., 2019 January-December, 33; as well as the further references cited therein.

As mentioned in these references, neuropathic pain (also abbreviated as “NP”) is caused by a lesion or disease of the somatosensory system, including peripheral fibers (A3, A6 and C fibers) and central neurons. Multiple causes of neuropathic pain have been described, including metabolic diseases such as diabetes; cancer and cancer treatments such as chemotherapy; neurological conditions such as those caused by autoimmune diseases (e.g. multiple sclerosis); neurodegenerative conditions such as Parkinson's disease; stroke; neuropathy caused by viral infections such as those caused by Herpes viruses (e.g. shingles); leprosy; Guillain-Barre syndrome; HIV infections; blood vessel diseases and vascular malformations; autoimmune conditions; and injury to nerves or the nervous system.

Accordingly, chronic neuropathic pain can originate from the peripheral part of the nervous system (for example in the case of trigeminal or post-herpetic neuralgia, peripheral nerve injury, painful polyneuropathies, or radiculopathies) or can originate from or involve the central nervous system (for example in the case of chronic neuropathic pain that develops as a result of spinal cord or brain injury, stroke or multiple sclerosis). As mentioned, when it originates from nerves beyond the brain and the central nervous system, it is also referred to as “peripheral neuropathy” or “neuropathy” for short.

As mentioned by Szok et al., supra, the International Association for the Study of Pain (IASP) in 2019 published a classification of these heterogeneous pain syndromes, with the following subtypes being recognized:
subtypes of chronic peripheral NP: trigeminal neuralgia (TN), chronic NP after peripheral nerve injury, painful polyneuropathy, post-herpetic neuralgia, and painful radiculopathy.
subtypes of chronic central NP: chronic central NP associated with spinal cord injury (SCI), chronic central NP associated with brain injury, chronic central post-stroke pain, and chronic central NP associated with multiple sclerosis.

As also mentioned by Szok et al.: “In general, NP conditions are underrecognized, underdiagnosed, and undertreated”.

The angiotensin II receptor type 2 (also referred to herein as “AT2R”) has been proposed as a target for the treatment of neuropathic pain. Reference is for example made to Shepherd et al., PNAS, vol. 115, no. 34, E8057-E8066 and Keppel Hesselink and Schatman, Journal of Pain Research, 2017, 10: 439-443; and Matavelli and Siragy, J. Cardiovasc. Pharmacol., 2015; 65(3): 226-232 and WO 2015/003223. Some of the known modulators of AT2R that have been proposed or investigated for the treatment of neuropathic pain include the compounds EMA200 (also known as PD-123319), EMA300, EMA400 (also known as PD-126055) and EMA401/olodanrigan (see for example Smith et al., Pain Medicine 2013, 14: 692-705; Anand et al., Mol. Pain 2015, 11: 38, as well as WO 2006/066361); the compound PD-123177 (see for example Singh and Karnik, J. Cell. Signal., 2016 June, 1(111)); and the compound known as C-38 (see for example Wallinder et al., ACS Med. Chem. Lett., 2015, 6, 2, 178-182; and Isaksson et al., Chemistry Open 2019, 8(1), 114-125.

The mechanism behind the analgesic properties of AT2R antagonists (such as EMA-401 referred to below) remains to be further elucidated. Some authors report that there are indications from pre-clinical studies that the analgesic properties of AT2R antagonists may be explained through a mechanism involving the modulation of macrophage-mediated neuro-immune interactions (Shepherd et al., J. Neurosci., 2018, 38(32): 7032-7057 and Shepherd et al., Proc. Natl. Acad. Sci. USA, 2018 Aug. 21; 115(34): E8057-E8066); whereas other authors have suggested, based on the finding that AT2R is co-located with TRPV1 in human dorsal root ganglions (DRGs), that AT2R plays a role in nociception by capsaicin sensitive sensory neurons and that AT2R antagonists may inhibit pain responses as well as neurite outgrowth in such cells (Anand et al., Eur. J. Pain, 17, (2013), 1012-1026).

AT2R and its ligand angiotensin II are well known in the art. Reference is for example made to Matavelli and Siragy, J. Cardiovasc. Pharmacol., 2015; 65(3): 226-232; Kaschina et al., Pharmacological Research 125 (2017), 39-47 (review); Berk, Science's STKE, 2003, Vol. 2003, Issue 181, pp. pe16; Juillerat-Jeanneret, J. Med. Chem., 2020, 63, 5, 1978-1995; Zhang et al., Nature, 2017; 544(7650): 327-332; Kemp et al., Circ. Res., 2014; 115(3): 388-table 1399; Namsolleck et al., Curr. Hypertens. Rep., (2014) 16: 416; Steckelings et al., Curr. Opin. Pharmacol., 2011 April; 11(2): 187-92; Mehta and Griendling, Am. J. Physiol. Cell Physiol., 292: C82-C97, 2007; Carey and Padia, Endocrine Hypertension, Volume 19, ISSUE 3, P84-87, Apr. 1, 2008; and Singh and Karnik, supra; as well as some of the further references cited herein.

As described in these references, AT2R is a G-protein coupled receptor that forms part of the renin-angiotensin system (RAS), a system that comprises multiple enzymes, peptide hormones and receptors and that is known, amongst its various biological functions, to be a major regulatory element in the control of cardiovascular and renal function.

Some of the main receptors involved in the RAS are the angiotensin II receptor type 1 (AT1R), angiotensin II receptor type 2 (AT2R), the Ang IV receptor (also known as AT4R), the pro(renin) receptor and the MAS receptor. Some of the native peptide ligands that are known to be involved in the RAS are the angiotensins, the ligands of the pro(renin) receptor (such as renin and prorenin) and ligands of the MAS receptor.

The angiotensins include the octapeptide Ang II and its natural degradation product Ang III (which are both known to be ligands for AT1R and AT2R), the hexapeptide Ang (1-7) (which is known to be the endogenous ligand for the MAS receptor), Ang IV (a natural degradation product of Ang II that is the main ligand of AT4R) and Ang I, a decapeptide precursor of Ang II that itself appears to have no major known direct biological activity.

Ang II, which has been described as the pivotal peptide hormone of the RAS, is known to be a potent pressor hormone and a primary regulator of aldosterone secretion by the adrenal cortex to promote sodium retention by the kidneys. As such, it is an important effector controlling blood pressure and volume in the cardiovascular system. Ang II is also used as a medication (marketed under the brand name GIAPREZA™) for treatment of vasodilatory shock.

Ang II and its natural degradation product Ang III are known to act (mainly) through AT1R and AT2R, which are both G protein-coupled receptors with about 34% sequence identity with each other. The actions of AT1R and AT2R are generally assumed to oppose each other. Activation of AT1R has been described as inducing biological actions such as, without limitation, cellular dedifferentiation and growth, vasoconstriction, antinatriuresis, aldosterone secretion, and sympathetic activation that ultimately lead to hypertension. Activation of AT2R has been described as inducing, among other biological effects, cellular differentiation and growth inhibition/apoptosis, vasodilation and natriuresis that potentially lower blood pressure leading to antihypertensive effects in renal disease, and as having a protective role with respect to various tissues and organs.

AT2R is known to be highly expressed in fetal tissue, including fetal aorta, gastrointestinal mesenchyme, connective tissue, skeletal system, brain, adrenal medulla and fetal kidney tissue. AT2R expression generally declines after birth, with significant levels in adults mainly in the myometrium and with lower levels in the adrenal gland and fallopian tube. Otherwise, in the tissues of healthy adults, the expression levels of AT2R are generally low, but AT2R is known to be strongly upregulated under pathological conditions such as tissue damage and injury (including vascular injury, neuronal injury, myocardial infarction and brain ischemia), where AT2R is thought to provide an endogenous protection to inflammatory, oxidative and apoptotic processes, again mainly by antagonizing AT1R.

In particular, as described by Anand (supra), “AngII and AT2R are co-expressed in nociceptive human sensory neurons, and the levels of AngII, the major endogenous ligand in human peripheral nerves, are preserved after injury. [. . . ] Hence increased AngII/AT2R signaling in DRG neurons secondary to peripheral nerve injury may have a key role in chronic pain mechanisms, including neuropathic pain.” Anand also hypothesizes that the mode of action of the known AT2R antagonist EMA401 “appears to involve inhibition of augmented AngII/AT2R induced p38 and p42/p44 MAPK activation, and hence inhibition of DRG neuron hyperexcitability and sprouting of DRG neurons”. Similarly, Smith et al. (2013), supra, describe that an analgesic dose of the AT2R antagonist EMA300 blocks augmented angiotensin II/AT2R signaling in the dorsal root ganglions which in turn inhibits p38 MAPK and p44/p42 MAPK activation in the ipsilateral lumbar DRGs of nerve-injured rats.

Despite antagonists of AT2R such as EMA401 showing promise in the treatment of post-herpetic neuralgia in human subjects (see for example Rice et al., The Lancet, Volume 383, P1637-1647, May 10, 2014) and in pre-clinical pain models of shingles, diabetes, osteoarthritis, HIV and chemotherapy (see for example WO 2006/066361 and Anand et al., supra), it would appear that the known AT2R antagonists have so far found limited success in their (further) clinical development.

Rice et al., Pain, 2021, March, 1 describe the results of two multicentre, randomised, double-blind treatment Phase 2b studies on the analgesic efficacy and safety of EMA401 in patients with post-herpetic neuralgia and painful diabetic neuropathy. As mentioned by Rice et al.: “The primary outcome for both the studies was change in weekly mean of the 24-hour average pain score, using a numeric rating scale (NRS), from baseline to Week 12. Both the studies were prematurely terminated due to preclinical hepatotoxicity on long-term dosing, although not observed in these studies.”. Rice et al. further state that “[ . . . ] as the studies were terminated prematurely, no firm conclusion could be drawn but the consistent clinical improvement in pain intensity reduction across these two studies in two different populations is worth noting.”

As will be clear from the above, there remains a constant need in the art for compounds that can be used in the prevention and/or treatment of pain, and in particular of chronic pain such as neuropathic pain. In the words of Rice et al.: “Existing treatments for peripheral neuropathic pain (PNP) have modest efficacy and are often not well tolerated, and the development of improved treatments for these common chronic pain conditions is recognised as a significant unmet need”.

In particular, there is a need for novel classes of molecules that can be used to target AT2R and/or to modulate the interaction(s) of AT2R with one or more of its ligands (such as Ang II) and that can be used in the prevention and/or treatment of various forms of chronic pain, such as the chronic pain states that are caused by and/or associated with damage to and/or disease of the somatosensory nervous system (and in particular damage to and/or disease of the peripheral nerves), the chronic pain states that are caused by and/or associated with hypersensitivity to pain in response to tissue damage and/or inflammation, and more generally the chronic pain states that are associated with AT2R-mediated signaling, Ang II-mediated activation of AT2R and/or Ang II-mediated activation signaling pathways involving p38 MAPK and/or p44/p42 MAPK.

### DESCRIPTION

The invention generally aims to meet this need by providing compounds that can interact with AT2R. In particular, the invention generally aims to provide compounds that can modulate (as defined herein) AT2R and AT2R-mediated signaling, such as the AT2R-mediated signaling that is associated with binding to AT2R of an AT2R ligand (such as a natural ligand of AT2R such as Ang II).

The compounds provided by the invention (which are also referred to herein as the “compounds of the invention”) are as further described herein.

Without being limited to any particular explanation, hypothesis or mechanism-of-action it is generally assumed that the compounds of the invention are capable of binding to AT2R in a manner that allows the compounds of the invention to compete for binding to AT2R with one or more ligands of AT2R (and in particular one or more natural ligands of AT2R such as Ang II).

Also, generally, the compounds of the invention have favorable selectivity for AT2R (e.g. compared to AT1R).

Furthermore, generally, based on their overall chemical structures, the compounds of the invention are assumed to comprise a pharmacophore that is favorable for clinical applications and therapeutic uses in humans and other mammals. It is also expected that the compounds of the invention may have certain advantages in terms of safety and tolerability compared to some of the known AT2R modulators described in the art.

Also, without being limited to any specific explanation, hypothesis or mechanism-of-action, it will be clear from the present description that the compounds of the invention are generally assumed to be capable of interacting with angiotensin-type receptors, and in particular with the angiotensin II receptor type 2.

In particular, it is assumed that the compounds of the invention are capable of modulating (as defined herein), and in particular specifically modulating (as defined herein), such as inhibiting, the angiotensin II receptor type 2, AT2R-mediated signaling and/or the pathways and/or biological processes in which AT2R and/or AT2R-mediated signaling is involved. More in particular, as mentioned herein, it is assumed that the compounds of the invention are capable of competing for binding to AT2R with one or more natural ligands of AT2R.

Thus, it is expected that the compounds and composition thereof can also be used for the prevention and treatment of diseases and disorders that can be prevented or treated by modulating, in a subject in need thereof, the angiotensin II receptor type 2, AT2R-mediated signaling and/or the pathways and/or biological processes in which AT2R and/or AT2R-mediated signaling is involved, in particular by administering, to said subject, one or more pharmaceutically active amounts (e.g. doses) of a compound of the invention, according to a suitable treatment or dosage regimen (which can be determined by the treating physician based on the state of the patient, the nature of the disease involved, the severity of the disease and/or its symptoms, and other factors that can be determined and suitably taken into account by the treating physician).

Thus, it is further expected that the compounds and composition can be used for the prevention and treatment of diseases and disorders that can be prevented or treated by administering, to a subject in need thereof, a compound that is capable of competing with the binding of one or more natural ligands to the angiotensin II receptor type 2, in particular by administering, to said subject, one or more pharmaceutically active amounts (e.g. doses) of a compound of the invention, according to a suitable treatment or dosage regimen (which can be determined by the treating physician based on the state of the patient, the nature of the disease involved, the severity of the disease and/or its symptoms, and other factors that can be determined and suitably taken into account by the treating physician). Without being limited to any specific explanation, hypothesis or mechanism-of-action, it will be clear to the skilled person that, usually, administration to a subject of a compound that is capable of competing with the binding of one or more natural ligands to the angiotensin II receptor type 2 will result in reducing the AT2R-mediated signaling that is associated with the binding of said ligand(s) to AT2R and/or inhibiting and/or antagonizing the pathways and/or biological processes that are triggered and/or activated by the binding of said ligand(s) to AT2R.

Examples of such diseases and disorders that are associated with AT2R, AT2R-mediated signaling, the pathways and/or biological processes in which AT2R and/or AT2R-mediated signaling is involved and/or binding of one or more natural ligands to AT2R will be clear to the skilled person (for example, from the prior art cited herein) and include other diseases and disorders for which the use of known modulators (and in particular modulators that compete for ligand binding to AT2R) and/or the use of known inhibitors and/or antagonists of AT2R and/or AT2R-mediated signaling have been described in the art.

Reference is for example also made to the listing of AT2R-mediated diseases and disorders given in WO 2019/179515. Reference is also made to Bonas-Guarch et al., Nat. Commun., 2018 Jan. 22; 9(1): 321 and Dominguez-Cruz et al., Gene, 2018 Nov. 30; 677: 324-331, which state the following on the involvement of AT2R in diabetes, in particular type II diabetes: “This rare variant identified in Xq23 chromosome was located near the AGTR2 gene, and showed nearly twofold increased risk for T2D in males, which represents, to our knowledge, the largest effect size identified so far in Europeans, and a magnitude similar to other variants with large effects identified in other populations. Using binding and gene-reporter analyses, we demonstrated a functional role of this variant and proposed a possible mechanism behind the pathophysiology of T2D in T risk allele carriers, in which this rare variant could favor a gain of function of AGTR2, previously associated with insulin resistance. AGTR2 appears, therefore, as a potential therapeutic target for this disease, which would be in line with previous studies showing that the blockade of the renin-angiotensin system in mice and in humans prevents the onset of T2D, and restores normoglycemia”; and “This gene encodes the receptor coupled to a G protein that helps the angiotensin II (Ang II) mediate its actions (Harrison-Bernard, 2009). Furthermore, the AGTR2 gene is a modulator of insulin sensitivity, and previous studies have showed a blockade of the renin-angiotensin system in mice (Frantz et al., 2013; Leung, 2007); in humans it prevents the onset of T2D and restores normoglycaemia (Geng et al., 2013).” Thus, it is expected that the compounds of the invention can be used in the prevention and treatment of diabetes, in particular type II diabetes.

The invention further relates to compositions, and in particular pharmaceutical compositions, that comprise at least one compound of the invention. These compositions, which are as further described herein, are also referred to as “compositions of the invention”.

The invention in particular relates to uses of the compounds and compositions of the invention in the prevention, treatment and/or management of pain, in particular chronic pain, such as various forms of neuropathic pain and/or inflammatory pain, as further described herein, as well as other diseases or disorders that are associated with AT2R and/or AT2R-mediated signaling (as further described herein).

These and other features, aspects, embodiments, uses and advantages of the present invention will become clear from the further description herein.

WO 2020/112905 describes compounds and methods for treating diseases using inhibitors of mono-acylglycerol lipase (MAGL), in which the compounds have the following formula, in which R1 is a —C(O)OR¹⁵ group (with R¹⁵ being hydrogen or C₁-C₆ alkyl) or a —C(O)NR¹⁰R¹¹ group (with R¹⁰ and R¹¹ each independently being hydrogen or C₁-C₆ alkyl).

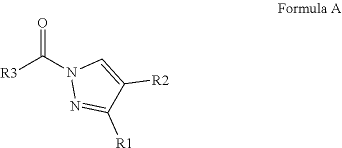

Among the diseases mentioned are atopic dermatitis, bladder dysfunction associated with multiple sclerosis, cardiovascular disease, contact dermatitis, cystic fibrosis, dermatomyositis, eczema, endometriosis, enteritis, fibromyalgia, Tourette syndrome, inflammatory bowel disease, interstitial cystitis, irritable bowel syndrome, ischemia, labor, abdominal pain, abdominal pain associated with irritable bowel syndrome, acute pain, back pain, cancer pain, chest pain, functional chest pain, joint pain, menstrual pain, metabolic disorders, musculoskeletal diseases, neuropathy, osteoarthritis, pancreatitis, pharyngitis, post-mastectomy pain syndrome, post-trigeminal neuralgia, post-operative pain, renal ischemia, rheumatoid arthritis, skeletal muscle contusion, skin diseases, sunburn, systemic lupus erythematosus, toothache, vaso-occlusive painful crises in sickle cell disease, and visceral pain.

Among the various MAGL inhibitors for which detailed chemical structures are listed, WO 2020/112905 in paragraph [00118] on page 53 describes a compound having the formula:

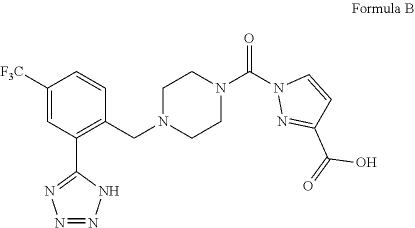 [SMILES: O=C(O)c1ccn(C(=O)N2CCN(Cc3ccc(C(F)(F)F)cc3-c3nnn[nH]3)CC2)n1]

No activity against AT2R is described or suggested. Also, as can be seen from Formula B, said MAGL inhibitor carries a substituent (i.e. a CF₃ group) on a position meta relative to the tetrazole group, and there are linking groups between both the piperazine ring and the tetrazole-substituted phenyl ring (i.e. a methylene linking group) as well as the piperazine ring and the carboxyl-substituted pyrazole group of Formula A. Also, in the compounds of Formulae A and B, the substituted pyrazole group is linked to the rest of the molecule via a nitrogen atom in the pyrazole ring and not a carbon atom.

In the present description and claims:
when a term is not specifically defined herein, it has its usual meaning in the art, which will be clear to the skilled person. Reference is for example made to the standard handbooks in the field of organic and/or medicinal chemistry, such as Karrer, Organic Chemistry, 3^{rd} Ed, 1947; Vogel, Practical Organic Chemistry, 3^{rd} Ed, 1964; Roberts and Caserio, Basic Principles of Organic Chemistry, 1^{st} Ed, 1965; Carey and Sundberg, Advanced Organic Chemistry, 2^{nd} Ed, 1985; Michael B. Smith, March's Advanced Organic Chemistry: Reactions, Mechanisms, and Structure, 1^{st} Ed, 2020; Jonathan Clayden, Nick Greeves, Stuart Warren, Organic Chemistry, 2^{nd} Ed, 2012; D. Sriram, Medicinal Chemistry, 2^{nd} Ed, 2010; and Camille Georges Wermuth, David Aldous, Pierre Raboisson, The Practice of Medicinal Chemistry, 4^{th} Ed, 2015; as well as to the “Glossary of terms used in medicinal chemistry (IUPAC Recommendations 1998)” by Wermuth et al., Pure and Applied Chemistry, 70 (5): 1129-1143 (1998).
when certain aspects are indicated as being preferred (for example, as being preferred for a certain part or structural element of a compound of the invention and/or the presence of a certain preferred substituent or combination of substituents), and such an aspect can be suitably combined with another aspect that is described as being preferred (for example, as being preferred for another part or structural element of a compound of the invention and/or the presence of another preferred substituent or combination of substituents), then the combination of such preferred aspects forms another preferred aspect of the invention. It should also be understood that the same applies, mutatis mutandis, to aspects that are described herein as being “more preferred”, “particularly preferred”, “even more preferred”, “most preferred” or by means of similar/equivalent wording;
when a structural formula is used to schematically illustrate or define a compound of the invention or an aspect or embodiment of the invention, and said structural formula shows an atom for which not all (required or possible) bonds/groups/substituents are shown, then it should be understood that the bonds/groups/substituents that are not explicitly shown can be in accordance with the further description herein of said compound/aspect/embodiment, and in the absence of such further description can be hydrogen or can each optionally be suitably and independently chosen from a suitable substituent (as defined herein), but will usually be a hydrogen atom;
when reference is made to a “suitable substituent” or where it is stated that an atom or position in a compound of the invention may be “suitably substituted”, each of these substituents (and the combination thereof) can be suitably chosen by the skilled person, optionally after a limited degree of trial-and-error. Suitable substituents will be clear to the skilled person based on the disclosure herein (optionally after a limited amount of trial-and-error) and for example include the substituents or combination of substituents that are mentioned herein and/or that are present in the compounds of the invention that are illustrated in the Experimental Part below (see compounds A-01 to A-251 in Table 1). Other suitable substituents will be clear to the skilled person based on the disclosure herein, and for example include (but are not limited to) halogen (i.e. F, Cl, Br and I), C₁-C₈ alkyl (in particular C₁-C₄ alkyl), C₁-C₈ alkoxy (in particular C₁-C₄ alkoxy), C₁-C₈ amine (in particular C₁-C₄ amine), cyclopropyl, cyclobutyl, trifluoromethyl (CF₃) and cyano, as well as for example oxygen (i.e. so as to form a carbonyl group); and may for example also include one or more of the substituents that are present in the compounds of the invention that are exemplified in the Experimental Part below (see the relevant substituents or combinations of substituents present in compounds A-01 to A-251 in Table 1);
when a particular position in a compound of the invention (and in particular a carbon atom or nitrogen atom that is present at said position) is said to be “optionally substituted”, the atom at that position can be unsubstituted or suitably substituted with 1, 2 and up to 3 (in the case of a carbon atom) or 1 or 2 (in the case of a nitrogen atom) substituents, which are suitably and each independently chosen from one or more suitable substituents (as defined herein). The maximum number of such suitable substituents that can be present at a particular position in a compound of the invention will depend on how many hydrogen atoms are carried by the atom that is present at said position and that can be suitably replaced by a suitable substituent (as defined herein), also taking into account factors such as the other atoms in the compound of the invention to which the atom in said position is bound. Also, as will be clear, the total number of substituents that can be present in or on a compound of the invention (or any structural part thereof) will generally depend on the number of atoms in such a compound that are (suitably) available for such substitutions (e.g. that carry a hydrogen atom that can be suitably replaced by such a substituent);
each compound of the invention can optionally be in the form of a suitable salt or ester (as further described herein), and in particular in the form of a pharmaceutically acceptable salt or ester (also as further described herein). Examples of suitable salts will be clear to the skilled person and include, but are not limited to, those mentioned in the further description herein;
a compound of the invention (including any salt or ester thereof) can generally be in any suitable or desired physical form, for example in a suitable crystalline form (including co-crystal forms or co-crystal salt forms), in a suitable amorphous form, and/or in the form of an anhydrate or a suitable hydrate or solvate (including but not limited to a hydrate or hemi-hydrate);
a compound of the invention (including or a salt or ester thereof) can generally also be present in a solution, for example in a suitable solvent or mixture of solvents, such as a suitable aqueous solvent or buffer. For pharmaceutical use, a compound of the invention can in particular be present in a pharmaceutically acceptable solution, which will usually be (sterile) water or a pharmaceutically acceptable aqueous buffer or solution. Suitable examples of such pharmaceutically acceptable aqueous buffers or solutions will be clear to the skilled person and include, but are not limited to, those mentioned in the further description herein;
a compound of the invention (or a salt or ester thereof) can generally be in pure or essentially pure form, in isolated or essentially isolated form, or in a suitable mixture with one or more other compounds;
where a compound of the invention contains one or more chiral atoms or centers, it can be in the form of one or more different optical forms, such as in the form of different enantiomers or different stereoisomers (e.g. in the form of two or more enantiomers or in the form of two or more diastereomers, with the term “diastereomer” or “diastereoisomer” generally being used herein to refer to any stereoisomer that is not an enantiomer). Generally, and unless explicitly indicated otherwise or when the specific context requires otherwise, when a compound of the invention can exist as one or more such optical forms, the term “compound of the invention” as used herein refers to and encompasses all such possible optical forms (e.g. all possible enantiomers or stereoisomers, respectively). Also, when a compound of the invention can exist in one or more such optical forms, the term “compound of the invention” encompasses each of such optical forms in essentially pure form (and/or in essentially isolated form) as well as mixtures of two or more such possible optical forms (in any ratio), including but not limited to racemic mixtures.
a compound of the invention can also be (administered or used) in the form of a suitable prodrug, e.g. a compound that, upon administration to a subject and/or under physiological conditions, is converted into a compound of the invention. Reference is for example made to IYAKUHIN no KAIHATSU (Development of Pharmaceuticals), Vol. 7, Design of Molecules, p. 163-198, Published by HIROKAWA SHOTEN (1990).
a compound of the invention may be suitably labeled with a suitable label (e.g. an atom, group, moiety or entity that, under suitable conditions, can (be used to) generate a detectable signal. Some specific but non-limiting aspects of suitable labels include radio-isotopes (e.g., ²H, ³H, ¹³C, ¹⁴C, ¹⁸F, ³⁵S ¹²⁵I) and fluorescent or phosphorescent labels. Such labeled compounds can be used for purposes known per se, such as for tissue distribution/penetration studies and for PK and other pharmacological studies and in the field of medical diagnosis and the like, using a suitable technique for detecting the label. For example, radiolabeled compounds of the invention may be used as a tracer in Positron Emission Tomography (PET) techniques;
as will be clear to the skilled person from the further description herein, it is possible that, in some cases as further indicated herein, different parts or structural elements of a compound of the invention are either directly linked (i.e. via a covalent bond) or alternatively linked via a suitable linking group or bridging group. When such a linking group (also referred to herein as an “alkylene linking group”) is present it will generally be an C₁ or C₂ alkylene group (and is preferably a methylene group, also referred to herein as a “methylene linking group”), in which each carbon atom in said alkylene linking group can independently optionally be suitably substituted (such as with one or two methyl groups per carbon atom) but is preferably unsubstituted (i.e. a —CH₂— linking group). Alternatively, such an alkylene linking group may be a carbonyl (i.e. a —(C═O)—) group, as further described herein;
when an atom is substituted with a fluor atom, said substituent will also be referred to as a “fluorine” or “fluoro” substituent (with similar terminology also being applied where a carbon atom is substituted with another halogen such as chlorine or bromine);
the term “bioisostere”, when used in respect of a compound, group or moiety, respectively, has its usual meaning in the art and as such refers to compounds, groups or moieties that have similar physical or chemical properties and as a result have broadly similar biological activities than said compound, group or moiety (Friedman HL (1951), NASNRS 206: 295-358). Bioisosteres, which are usually obtained by replacing an atom or group of atoms with another, broadly similar, atom or group of atoms, may have one or more enhanced properties (such as reduced toxicity, improved bioavailability, modified or improved activity, or an improved metabolic profile) compared to the original compound, group or moiety. Reference is for example made to Meanwell, Tactics in Contemporary Drug Design pp. 283-380, Topics in Medicinal Chemistry, Volume 9, 2014;
the term “pharmacophore” generally refers to the commonly used IUPAC definition, i.e. “an ensemble of steric and electronic features that is necessary to ensure the optimal supramolecular interactions with a specific biological target and to trigger (or block) its biological response”. Reference is for example made to the “Glossary of terms used in medicinal chemistry (IUPAC Recommendations 1998)” by Wermuth et al., Pure and Applied Chemistry, 70 (5): 1129-1143 (1998);
the term “alkyl” is defined as a linear or branched saturated aliphatic hydrocarbon. In some embodiments, an alkyl group has from 1 to 12 carbon atoms, particularly from 1 to 8 (“C₁₋₈ alkyl” or “C₁-C₈ alkyl”), for example from 1 to 6 carbon atoms (“C₁₋₆ alkyl” or “C₁-C₆ alkyl”), such as 1 up to 4 carbon atoms (“C₁₋₄ alkyl” or “C₁-C₄ alkyl”), more particularly 1, 2, 3, 4, 5, 6, 7, or 8 carbon atoms. For example, as used herein, the terms “C₁₋₈ alkyl” and or “C₁-C₈ alkyl” refer to a linear or branched group of 1 to 8 carbon atoms (e.g. methyl, ethyl, n-propyl, isopropyl, n-butyl, isobutyl, sec-butyl, tert-butyl, n-pentyl, isopentyl, neopentyl or 1-hexyl, 2-hexyl, 3-hexyl, 2-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, etc.), which are optionally substituted by one or more (such as 1 to 3) suitable substituents such as halogen (also referred to as “haloalkyl”, e.g. CH₂F, CHF₂, CF₃, CCl₃, C₂F₅, C₂Cl₅, CH₂CF₃, CH₂Cl or CH₂CH₂CF₃, etc.). The terms “C₁₋₄ alkyl” and “C₁-C₄ alkyl” refer to a linear or branched aliphatic hydrocarbon chain of 1 to 4 carbon atoms (i.e. methyl, ethyl, n-propyl, isopropyl, n-butyl, isobutyl, sec-butyl or tert-butyl);
the term “C₁-C₈ alkoxy” refers to an alkoxy group that comprises a C₁-C₈ alkyl group as defined herein. Similarly, the term “C₁-C₆ alkoxy” refers to an alkoxy group that comprises a C₁-C₆ alkyl group as defined herein and the term “C₁-C₄ alkoxy” refers to an alkoxy group that comprises a C₁-C₄ alkyl group;
the term “C₁-C₈ amine” refers to an amine group NR_{F}R_{G} in which each of R_{F} and R_{G} is independently hydrogen or a C₁-C₈ alkyl group as defined herein (provided that R_{F} and R_{G} are not both hydrogen). Similarly, the term “C₁-C₆ amine” refers to an amine group NR_{F}R_{G} in which each of R_{F} and R_{G} is independently hydrogen or a C₁-C₆ alkyl group as defined herein (provided that R_{F} and R_{G} are not both hydrogen) and the term “C₁-C₄ amine” refers to an amine group NR_{F}R_{G} in which each of R_{F} and R_{G} is independently hydrogen or a C₁-C₄ alkyl group as defined herein (provided that R_{F} and R_{G} are not both hydrogen);
the term “cycloalkyl” refers to a saturated cycloalkyl group with between 3 and 6 carbon atoms (e.g. cyclopropyl, cyclobutyl, cyclopentyl or cyclohexyl), which may suitably be unsubstituted or substituted (as described herein), but which when substituted is most preferably suitably substituted with only C₁ or C₂ alkyl groups (and preferably 1 or 2 methyl groups);
the term “heterocyclyl” refers to “a non-aromatic ring with between 3 and 10 ring atoms, and preferably 3, 4, 5 or 6 ring atoms, which suitably contains carbon atoms and 1 or 2 hetero-atoms (each suitably chosen from N, O or S) such that each such hetero-atom is most preferably connected to two carbon atoms in the ring (and most preferably not to another hetero-atom in the ring), which non-aromatic ring may suitably be unsubstituted or substituted (as described herein), but which when substituted is most preferably suitably substituted with only C₁ or C₂ alkyl groups (and preferably 1 or 2 methyl groups). Some representative but non-limiting examples include pyrroline, pyrrolidine, pyrazolidine, imidazoline, tetrahydrofuran, piperidine, piperazine, morpholine. Each such heterocyclyl group, if present, may suitably be unsubstituted or substituted (as described herein), but which when substituted is most preferably suitably substituted with only C₁ or C₂ alkyl groups (and preferably 1 or 2 methyl groups). Also, when such a heterocyclyl group contains one or more nitrogen atoms, each such nitrogen atom may be unsubstituted or suitably substituted with an C₁-C₃ alkyl group (and preferably a methyl group);
the terms “modulate”, “modulation”, “modulator” and “target” will have their usual meaning in the art, for which reference is inter alia made to the definitions given in WO 98/06737. Generally, in the context of the present invention, “modulating” or “to modulate” generally means either reducing or inhibiting the activity of, or alternatively increasing the activity of, a GPCR, as measured using a suitable in vitro, cellular or in vivo assay (such as those mentioned herein). In particular, “modulating” or “to modulate” may mean either reducing or inhibiting the activity of, or alternatively increasing the activity of, a GPCR, as measured using a suitable in vitro, cellular or in vivo assay (such as those mentioned herein), by at least 1%, preferably at least 5%, such as at least 10% or at least 25%, for example by at least 50%, at least 60%, at least 70%, at least 80%, or 90% or more, compared to activity of a GPCR in the same assay under the same conditions but without the presence of the compound, amino acid sequence or polypeptide of the invention (as applicable). As will be clear to the skilled person, “modulating” may also involve effecting a change (which may either be an increase or a decrease) in affinity, avidity, specificity and/or selectivity of a GPCR for one or more of its targets, ligands or substrates; and/or effecting a change (which may either be an increase or a decrease) in the sensitivity of a GPCR for one or more conditions in the medium or surroundings in which a GPCR is present (such as pH, ion strength, the presence of co-factors, etc.), compared to the same conditions but without the presence of the compound, amino acid sequence or polypeptide of the invention (as applicable). As will be clear to the skilled person, this may again be determined in any suitable manner and/or using any suitable assay known per se, such as the assays described herein or in the prior art cited herein. “Modulating” may also mean effecting a change (i.e. an activity as an agonist, as an antagonist, as a reverse agonist and/or as an allosteric modulator, respectively, depending on the GPCR and the desired biological or physiological effect) with respect to one or more biological or physiological mechanisms, effects, responses, functions, pathways or activities in which a GPCR (or in which its substrate(s), ligand(s) or pathway(s) are involved, such as its signaling pathway or metabolic pathway and their associated biological or physiological effects) is involved. Again, as will be clear to the skilled person, such an action as an agonist or an antagonist may be determined in any suitable manner and/or using any suitable (in vitro and usually cellular or in vivo) assay known per se, such as the assays described herein or in the prior art cited herein. In particular, an action as an agonist or antagonist may be such that an intended biological or physiological activity (such as, for example and without limitation, receptor mediated signaling) is increased or decreased, respectively, by at least 1%, preferably at least 5%, such as at least 10% or at least 25%, for example by at least 50%, at least 60%, at least 70%, at least 80%, or 90% or more, compared to the biological or physiological activity in the same assay under the same conditions but without the presence of the relevant compound, amino acid sequence or polypeptide of the invention. Generally, in the context of the present description and claims, it should be understood that the present invention and disclosure are not limited to any specific mechanism by which a compound, amino acid sequence or polypeptide of the invention modulates its target (as long as the target is modulated as described herein), and thus that modulating may, for example and without limitation, involve binding at an orthosteric site or allosteric modulation (i.e. binding at an allosteric site, see for example George et al., Nat. Rev. Drug Discov. 1: 808-820 (2002); Kenakin, Trends Pharmacol. Sci. 25: 186-192 (2002) and Rios et al., Pharmacol. Ther. 92: 71-87 (2001)), reducing or inhibiting the binding of a GPCR to one of its substrates or ligands and/or competing with a natural ligand or substrate for binding to the GPCR. Modulating may also involve activating a GPCR or the mechanism or pathway in which it is involved. Modulating may be reversible or irreversible, but for pharmaceutical and pharmacological purposes will usually be in a reversible manner. Also, generally, a “modulator” is a compound or factor that can modulate a GPCR, which as set out herein means that it can, under physiological conditions and/or the conditions used in a relevant assay or model, enhance, inhibit/reduce or otherwise alter, influence or affect a functional property of a biological activity or process (for example, the biological activity of a target and/or of the signaling and/or biological pathways in which said target is involved, or a biological read-out associated with said target or signaling). In this context, a modulator may be used to modulate a target in vitro (e.g. as part of an assay or screen) and/or in vivo (for example, when the modulator is administered to an animal (e.g. for veterinary purposes or as part of an in vivo model), a subject (i.e. for therapeutic purposes, i.e. the prevention or treatment of one or more diseases in said subject that can be prevented or treated through the use of said modulator). Modulators or candidate modulators may for example be identified, tested and/or further characterized by means of an in vitro screen or assay that involves the relevant target, such as a primary screen (e.g. a screen used to identify modulators of the target from a set or library of test chemicals with unknown activity with respect to the target) and/or a secondary assay (e.g. an assay used for validating hits from a primary screen and/or used in optimizing hit molecules, e.g. as part of hits-to-leads chemistry), and/or a cellular model or animal model that allows for one or more relevant parameters (i.e. the parameters to be modulated) to be tested/determined. For instance, such an assay or screen may be configured as an in vitro assay or screen, which will generally involve binding of the (candidate) modulator to the target, upon which a signal generated by said binding is measured. Suitable techniques for such in vitro screening will be clear to the skilled person, and are for example described in Eldefrawi et al., (1987). FASEB J., Vol. 1, pages 262-271 and Rauh et al., (1990), Trends in Pharmacol. Sci., vol. 11, pages 325-329. For example, such an assay or screen may be configured as a binding assay or screen, in which the (candidate) modulator is used to displace a detectable ligand from the target (e.g. a radioactive or fluorescent ligand), upon which the amount of ligand displaced from the target by the modulator is determined. Generally, a compound will be considered a modulator when it alters or changes said functional property of a biological activity or process (i.e. a signal or read-out in a relevant assay or model) by at least 1 percent, such as at least 5 percent or more, compared to the value(s) obtained when the same assay or model is performed without the presence of the (candidate) modulator:
the term “affinity” denotes the strength or stability of a molecular interaction. The affinity is commonly given by the K_{D}, or dissociation constant, which has units of mol/liter (or M). The affinity can also be expressed as an association constant, K_{A}, which equals 1/K_{D} and has units of (mol/liter)⁻¹ (or M⁻¹). Affinity can be determined in a manner known per se;
it should also be clear that, in the present description and claims, the term “monocyclic”, when this term refers to a ring system (such as an aliphatic ring system or an aromatic ring system), refers to a ring system that comprises or essentially consists of a single ring, which ring suitably comprises or essentially consists of carbon atoms and optionally one or more (such as 1 or 2) hetero-atoms (which hetero-atoms are preferably each independently and suitably chosen, more preferably from O, N and/or S). Depending on the atoms that are present in said ring, a monocyclic ring can suitably comprise or essentially consist of between 3 and 10 ring atoms, such as 3, 4, 5, 6, 7, 8, 9 or 10 ring atoms, and in particular 4, 5 or 6 ring atoms.

Similarly, in the present description and claims, the term “bicyclic”, when this term refers to a ring system (such as an aliphatic ring system or an aromatic ring system), refers to a ring system that comprises or essentially consists of two rings which are suitably fused with each other (usually such that the two fused rings share two ring atoms, in which each of the rings that form the bicyclic ring system suitably comprises or essentially consists of carbon atoms and optionally one or more (such as 1 or 2) hetero-atoms (which hetero-atoms are preferably each independently and suitably chosen, more preferably from O, N and/or S). Generally, and depending on the number of atoms that are present in each ring, a bicyclic ring can suitably comprise or essentially consist of between 8 and 12 atoms, such as 8, 9, 10, 11 or 12 ring atoms. The fused rings in a bicyclic ring system will usually share two carbon atoms, but as will be clear to the skilled person, it is also possible for the fused rings to share one carbon atom and one nitrogen atom if a nitrogen atom is suitably present.

It will also be clear to the skilled person that a bicyclic ring system can suitably comprise or essentially contain two aliphatic rings (again suitably fused, usually such that the two fused rings share two atoms), two aromatic rings (again suitably fused, usually such that the two fused rings share two atoms and that the totality of the two rings in the bicyclic rings system forms a conjugated planar ring system); or suitably comprise or essentially consist of one aromatic ring and one aliphatic ring (again suitably fused, usually such that the two fused rings share two atoms). Herein, for the sake of convenience only and without limiting the scope of the present disclosure or claims, a bicyclic ring system that comprises at least one aromatic ring will generally be referred to herein as an aromatic ring system, irrespective of whether the second ring in the ring system is aromatic or aliphatic. As will also be clear to the skilled person, a bicyclic ring system can also suitably comprise two of the monocyclic ring systems referred to herein, which are suitably fused to each other (i.e. such that in the bicyclic ring system, the two fused rings share at least two ring atoms).

Similarly, in the present description and claims, the term “polycyclic”, when this term refers to a ring system (such as an aliphatic or aromatic ring system), refers to a ring system that comprises or essentially consists of two or more rings (such as 2, 3, 4 or 5 rings), in which each ring within the ring system is suitably and independently fused to at least one other ring within the ring system (usually such that said two rings that are fused to each other share two rings atoms). Again, in a polycyclic ring system, each of the rings that form the ring system suitably comprises or essentially consists of carbon atoms and may optionally comprise one or more (such as 1 or 2) hetero-atoms (which hetero-atoms are preferably each independently and suitably chosen, more preferably from O, N and/or S). As will be clear to the skilled person, the total number of ring atoms in a polycyclic ring system will depend on the total number of rings in the ring system, the number of ring atoms in each ring, and the number of ring atoms that are shared between the different rings. The fused rings in a polycyclic bicyclic ring system will usually share two carbon atoms, but as will be clear to the skilled person, it is also possible for the fused rings to share one carbon atom and one nitrogen atom if a nitrogen atom is suitably present.

It will also be clear to the skilled person that a polycyclic ring system can suitably comprise or essentially contain only aliphatic rings, can suitably comprise or essentially contain only aromatic rings, or can suitably be comprised of one or more aliphatic rings and one or more aromatic rings (and again, for the sake of convenience only and without limiting the scope of the present disclosure or claims, a polycyclic ring system that comprises at least one aromatic ring will generally be referred to herein as an aromatic ring system, irrespective of whether the other rings in the system are aromatic and/or aliphatic). As will also be clear to the skilled person, a polycyclic ring system can also suitably comprise two or more of the monocyclic ring systems referred to herein, which are suitably fused to each other (i.e. such that in the polycyclic ring system, two rings that are fused to each other share at least two ring atoms).

It will also be clear that, in the context of the present description and claims, the term “tricyclic”, when this term refers to a polycyclic ring system as described herein, refers to a polycyclic ring system that essentially consists of three rings.

Also, in the present description and claims, the term “heterocyclic”, when this term refers to a ring system (such as an aliphatic or aromatic ring system), refers to a ring system that suitably contains one or more hetero-atoms (which are preferably each independently chosen from N, O or S); and the term “hetero-aromatic”, when this term refers to an aromatic ring system, refers to an aromatic ring system that suitably contains one or more hetero-atoms (which are preferably each independently chosen from N, O or S).

It should be noted that, in a compound of the invention that comprises a ring system as described herein:
the ring system may be covalently bound or linked to the other part (or parts) of the compound of the invention at any suitable position (or positions) of the ring system, also relative to any other structural elements that are present in or on the ring system, such as relative to any hetero-atoms that are present in the ring system and/or relative to any substituents that are present on the ring system. Also, where the ring system contains one or more nitrogen atoms, the ring system may be covalently bound or linked to one or more other parts and/or structural elements of the compound of the invention via said nitrogen atom; and/or
each of the atoms that make up said ring system may independently either not carry any substituents or may be suitably substituted (as defined herein), with the number of possible substituents on a given ring atom being dependent on how many further bonds a given ring atom can form in addition to the bonds it has to the other ring atoms (e.g. how many hydrogen atoms said ring atom carries that can be suitably replaced by a suitable substituent). Generally, if one or more such suitable substituents are present, they may be present at any suitable position or positions of the ring system, also relative to any other structural elements of the ring system, such as relative to any hetero-atoms that are present in the ring system and/or relative to the position (or positions) where the ring system is covalently linked to the other part (or parts) of the compound of the invention. Also, where the ring system contains one or more nitrogen atoms, each of said nitrogen atoms may independently either not be substituted or be suitably substituted (as defined herein), again depending on how many further bonds said nitrogen ring atom can form in addition to the bonds it has to the other ring atoms (e.g. how many hydrogen atoms said nitrogen atom carries that can be suitably replaced by a suitable substituent); and/or
where said ring system contains one or more hetero-atoms, each of said one or more hetero-atoms may be at any suitable position(s) in the ring system (i.e. relative to the carbon atoms in the ring system), also relative to any other structural elements that are present in or on said ring system, such as relative to any further hetero-atoms that may be present in the ring system, relative to any substituents that are present on the ring system and/or relative to the position (or positions) where the ring system is covalently linked to the other part (or parts) of the compound of the invention. When it is desired or intended that one or more rings of the ring system that contain one or more hetero-atoms should be aromatic rings, then the one or more hetero-atoms that are present in said ring(s) should be at position(s) relative to the carbon atoms in said ring(s) and any other hetero-atoms in said ring(s) such that the resulting ring(s) form a conjugated planar ring system. Also, where the ring system contains one or more nitrogen atoms, each of said nitrogen atoms may independently also be suitably substituted (as defined herein), again depending on how many further bonds said nitrogen ring atom can form in addition to the bonds it has to the other ring atoms.

It should further be clear that, in the present description and claims, the terms “aliphatic ring”, “aliphatic ring system” have their usual meaning in the art and generally refer to aliphatic compounds that comprise or essentially consist of one or more rings (such as a single ring or two or more fused rings). As used herein, and as customary in the art, the term aliphatic ring system also comprises rings and ring systems that contain one or more double or triple bonds, as long as said ring is not an aromatic ring (as defined herein).

Also, the term “alicyclic” is used herein to refer to a non-aromatic ring system that only comprises carbon atoms.

Generally, as will be clear to the skilled person, such an aliphatic ring system can be monocyclic, bicyclic, tricyclic or polycyclic (all as described herein) and can contain only carbon atoms or can suitably contain carbon atoms and a suitable number (such as 1, 2, 3 or more, depending on the total number of rings in the aliphatic ring system) of hetero-atoms that are each independently chosen from N, S or O. It should also be noted that each atom in such an aliphatic ring system can be suitably substituted (as defined herein). An aliphatic ring system can also suitably contain one or more double bonds (such as a carbon-carbon double bond), albeit that, for the purposes of convenience only and without limiting the scope of the present disclosure or claims, a ring system that contains at least one aromatic ring will generally be referred to herein as an aromatic ring system.

An aliphatic ring system as described herein may also be a bridged ring system, in which the terms “bridged” or “bridge” have their usual meaning in the art (i.e. meaning that such a bridged aliphatic ring system comprises two rings which share three or more atoms, separating the two bridgehead atoms by a bridge containing at least one atom, for example with an alkylene bridge as described herein). Such a bridged aliphatic ring system may also suitably contain one or more hetero-atoms. Some specific but non-limiting examples of bridged aliphatic ring systems include bicyclo[2.2.1]heptane and bicyclo[2.2.2]octane as well as the bridged ring systems that are present in the compounds of the invention that are exemplified in the Experimental Part below (see for example compounds A-189, A-190, A-232 and A-233). For the purposes of the present description and claims, a 6-, 7-, 8-, 9- or 10-membered ring in which there is a direct covalent bond between two carbon atoms in the ring such that resulting ring system essentially comprises two fused rings that share said two connected ring atoms (as for example in decalin or the bicyclic diaza-structure of formula XCVIII herein) will also be considered a “bridged” ring system, even though in such structures the “bridge” consists of a covalent bond.

Some specific, but non-limiting examples of aliphatic ring systems include, but are not limited to:
monocyclic aliphatic ring systems containing only carbon atoms: cycloalkanes such as cyclopropyl, cyclobutyl, cyclopentyl, cyclohexyl, cycloheptyl and cyclo-octyl, and cycloalkenes such as cyclopentene and cyclohexene;
monocyclic aliphatic ring systems containing carbon atoms and nitrogen atoms: azetidine, pyrrolidine, 2-pyrroline, 3-pyrroline, pyrazolidine, imidazolidine, 2-pyrazoline, 2-imidazoline, piperidine, piperazine;
monocyclic aliphatic ring systems containing carbon atoms and oxygen atoms: oxetane, tetrahydrofuran, 1,3-dioxolane, tetrahydropyran, 1,4-dioxane, 2H-pyran, 4H-pyran, 1,4-dioxine;
monocyclic aliphatic ring systems containing carbon atoms and sulfur atoms: tetrahydrothiophene (thiolane), 1,2-oxothiolane, 1,3-oxothiolane;
monocyclic aliphatic ring systems containing carbon atoms and two different hetero-atoms: 1,2-oxathiolane, 1,3-oxathiolane, morpholine, thiomorpholine, 1,2-thiazine, 1,4-thiazine;
bicyclic aliphatic ring systems containing carbon atoms and nitrogen atoms: pyrrolizidine, decahydroisoquinoline, decahydroquinoline,
bicyclic aliphatic ring systems containing carbon atoms and sulfur atoms;
bicyclic aliphatic ring systems containing carbon atoms and two or more different hetero-atoms.

Some other examples of aliphatic rings/ring systems will be clear to the skilled person based on the further disclosure herein and from the compounds of the invention exemplified in the Experimental Part below that contain such aliphatic rings/ring systems.

Again, when present in a compound of the invention, such an aliphatic ring system may be suitably covalently bound or linked to another part (or two or more other parts) of the compound of the invention at any suitable position of the ring system (again, dependent upon whether the atom that is present at said position can form a covalent bond with the other part(s) of the compound of the invention). Also, again, each of the atoms that make up the aliphatic ring system may independently either not carry any substituents or may be optionally substituted (as defined herein), with the number of possible substituents on a given ring atom being dependent on the number of covalent bonds that said ring atom can form in addition to its bonds that it has to other ring atoms.

It will also be clear that, in the present description and claims, the terms “aromatic ring” or “aromatic ring system” have their usual meaning in the art and generally refer to rings that form a conjugated planar ring system.

Generally, as will be clear to the skilled person, such an aromatic ring system can be monocyclic, bicyclic, tricyclic or polycyclic (all as described herein) and can contain only carbon atoms or can suitably contain a suitable number (such as 1, 2, 3 or more, depending on the total number of rings in the aromatic ring system) of hetero-atoms (which are preferably each independently chosen from N, S or O). It should also be noted that each atom in such an aromatic ring system can be suitably substituted (as defined herein). As also described herein, for the sake of convenience only, a polycyclic ring system that contains one or more aliphatic rings in addition to the one or more aromatic rings (for example xanthene) will be considered an aromatic ring system.

Some specific, but non-limiting examples of such aromatic ring systems include, but are not limited to:
monocyclic aromatic ring systems containing only carbon atoms: phenyl;
monocyclic aromatic ring systems containing carbon atoms and one or more nitrogen atoms: pyrrole, pyrazole, imidazole, 1,2,4-triazole, 1,2,3-triazole, tetrazole, pyridine, pyridazine, pyrimidine, pyrazine, 1,2,4-triazine, 1,3,5-triazine, azepine, 1,4-diazepine;
monocyclic aromatic ring systems containing carbon atoms and oxygen atoms: furan;
monocyclic aromatic ring systems containing carbon atoms and sulfur atoms: thiophene;
monocyclic aromatic ring systems containing carbon atoms and two different hetero-atoms: thiazole, oxazole, isoxazole, isothiazole, 1,2,3-oxadiazole, 1,2,4-oxadiazole, 1,2,5-oxadiazole, 1,3,5-oxadiazole;
bicyclic aromatic ring systems containing only carbon atoms: naphtalene, indene, 2,3-dihydro-indene;
bicyclic aromatic ring systems containing carbon atoms and nitrogen atoms: indole, isoindole, indoline, quinoline, isoquinoline, indolizine, indazole, benzimidazole, quinoxaline, phtalazine, quinazoline, cinnoline, 1,8-naphteridine, pyrido[3,2-d]-pyrimidine, pyrido[4,3-d]-pyrimidine, pyrido[3,4-b]pyrazine, pyrido[2,3-b]pyrazine, pteridine, 4-azaindole, 5-azaindole, 6-azaindole, 7-azaindole, purine, tetrahydroquinoline, 1,2-dihydroquinoline, 1,2-dihydroisoquinoline;
bicyclic aromatic ring systems containing carbon atoms and oxygen atoms: benzofuran, isobenzofuran;
bicyclic aromatic ring systems containing carbon atoms and sulfur atoms: benzo[c]thiophene, benzo[b]thiophene;
bicyclic aromatic ring systems containing carbon atoms and two or more different hetero-atoms: benzo[c]isoxazole, benzo[d]isoxazole, benzo[c]thiazole, benzo[d]thiazole, benzo[d]oxazole, benzo[e][1,2]oxazine, benzo[e][1,3]oxazine, benzo[b][1,4]oxazine;
tricyclic ring and polycyclic ring systems: anthracene, phenantrene, pyrene, benzo(a)pyrene, carbazole, alloxazine, acridin, phenazine, phenoxazine, phenothiazine.

Some other examples of aromatic rings/ring systems will be clear to the skilled person based on the further disclosure herein and from the compounds of the invention exemplified in the Experimental Part below that contain such aromatic rings/ring systems.

Again, when present in a compound of the invention, such an aromatic ring system may be suitably covalently bound or linked to another part (or two or more other parts) of the compound of the invention at any suitable position of the ring system (again, dependent upon whether said ring atom can form a covalent bond in addition to the bonds that link it to other ring atoms). Also, again, each of the atoms that make up the aromatic ring system may independently either not carry any substituents or may be optionally substituted (as defined herein), again, dependent upon whether said ring atom can form covalent bonds with such substituents in addition to the bonds that link it to other ring atoms.

It should also be noted, as will be clear to the skilled person, that a ring system as described herein may also be suitably substituted with one or more (but usually only one or two and often only one) oxygen atom. Some specific but non-limiting examples of ring systems that are substituted with an oxygen atom include, but are not limited to: quinolin-2-[1H]-one, isoquinolin-1-[2H]-one.

Other examples of ring systems that can be present in a compound of the invention will become clear to the skilled person based on the disclosure herein, and include the ring systems that are present in the compounds of the invention that are exemplified in the Experimental Part below.

As further described herein and as illustrated by some of the compounds of the invention that are exemplified in the Experimental Part below, when a compound comprises two or more rings or ring systems (which rings are not fused into a single ring system) said rings may be suitably either be directly linked via a covalent bond or indirectly linked via a suitable (unsubstituted or suitably substituted) alkylene linking group (as further defined herein), such as a (unsubstituted or suitably substituted) methylene linking group (in which case, as mentioned herein, each such alkylene linking group may alternatively be a carbonyl group).

As further described herein, as part of their overall structure, the compounds of the invention generally comprise an aliphatic ring (which aliphatic ring is as further described herein) that is covalently linked (i.e. either directly or via an alkylene linking group and in particular a methylene linking group, both as defined herein) to an aromatic ring (which aromatic ring is as further described herein), which aromatic ring further at least carries an acidic substituent (which an acidic substituent is as further described herein, and which, without being limited to any hypothesis or explanation, is thought to allow for interaction or improved interaction with AT2R and in particular with one or more of the amino acids in the AT2R sequence that are thought to be present in the putative binding pocket/binding site on/in AT2R to which the compounds of the invention are assumed to bind) on a carbon atom that is adjacent to (i.e. in the ortho-position relative to) the carbon atom of the ring to which the aliphatic ring is bound. This is schematically illustrated in the following scheme (Scheme A), in which the aliphatic ring is schematically exemplified using a 6-membered ring, the aromatic ring is schematically exemplified using a 6-membered ring (with the aromatic nature of the ring being schematically indicated using a dashed circle).

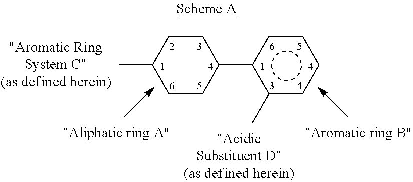

The above Scheme A also shows the numbering system for the atoms of said aliphatic ring and said aromatic ring which, for the sake of convenience only and without limiting the scope of the present disclosure or claims, will be used in the present description and claims.

Also, solely for the sake of convenience only and without limiting the scope of the present disclosure or claims, said aliphatic ring will also be referred to herein as “Aliphatic Ring A” or “Ring A” etc. and will also be denoted as/by “[A]”, said aromatic ring will also be referred to herein as “Aromatic Ring B” or “Ring B” etc. and will also be denoted as/by “[B]”, and said acidic substituent will also be referred to herein as the “Acidic Substituent”, “Substituent D” etc. and denoted as/by “[D]”.

With reference to the above Scheme A, in the further description and claims:
the position/atom in the Aromatic Ring B that is indicated as “3” in the above Scheme A will also be generally referred to as “a position/atom that is in the ortho-position relative to the Acidic Substituent”; and
the position/atom in the Aromatic Ring B that is indicated as “4” in the above Scheme A will also be generally referred to as “a position/atom that is in the meta-position relative to the Acidic Substituent”; and
the position/atom in the Aromatic Ring B that is indicated as “5” in the above Scheme A will also be generally referred to as “a position/atom that is in the para-position relative to the Acidic Substituent”.

It should be noted that, in the present description and claims, the wording “a position/atom that is in the meta-position relative to the Acidic Substituent” is used solely to indicate the position/atom on the Aromatic Ring B that is indicated as “4” in the above Scheme A (and not the position/atom on the Aromatic Ring B that is indicated as “6” in the above Scheme A, which will also be referred to herein as “the other ortho-position on the aromatic ring”).

With respect to the Aliphatic Ring A, it should also be noted that, as further described herein, the Aliphatic Ring A may also essentially consist of a bridged ring system (as further described herein), which may be unsubstituted or substituted (as further described herein); or a fused ring system (as further described herein), which may be unsubstituted or substituted (as further described herein); or a spiro-type ring system, which may be unsubstituted or substituted (as further described herein). Some specific but non-limiting examples of such a fused ring system or spiro-type ring system are given herein as Formulae LXXXV and XCII, Formula XCVIII and Formula C, respectively.

More generally, the Aliphatic Ring A may be a bioisostere (as defined herein) of the ring structure of Scheme C herein and in particular of the ring structure of Scheme D herein. Suitable bioisosteres will be clear to the skilled person based on the disclosure herein.

With respect to the Aromatic Ring B, it should be noted that, if said Ring B is a 5-membered ring, that the same numbering system will be used, with the position/atom to which the Aromatic Ring B is linked to the Aliphatic Ring A again being indicated as position “1” but without the position/atom that is indicated as “6” in the above Scheme A.

Similarly, with respect to the Aliphatic Ring A, it should be noted that, if said Ring A is a 5-membered ring, that the same numbering system as shown in Scheme A will be used, with the position/atom to which the Aromatic Ring System C is linked to the Aliphatic Ring A again being indicated as position “1” but without the position/atom that is indicated as “6” in the above Scheme A. It should also be noted, with respect to the Aliphatic Ring A, that if said Ring A is a 7-membered ring, that the same numbering system will be used, with the position/atom to which the Aromatic Ring System C is linked to the Aliphatic Ring A being indicated as position “1” and there being an additional position/atom in the Aliphatic Ring A that is present between position/atom “1” and position/atom “6” and that will be referred to herein as position/atom “7” (similarly, if Ring A is an 8-membered ring, there will be additional positions/atoms “7” and “8” between positions “1” and “6” as shown in Scheme A).

Also, with reference to the above Scheme A, and again for the sake of convenience only and without limiting the scope of the present disclosure or claims, in the further description and claims, the positions/atoms on the Aliphatic Ring A that are indicated as “1” and “4”, respectively, in Scheme A will also be referred to as positions/atoms in Ring A that are “opposite” to each other. Similarly, when the Aliphatic Ring A is a 5-membered ring, each of the positions/atoms “3” and “4” will be deemed opposite to the position/atom at position “1” in Ring A, and when the Aliphatic Ring A is a 7-membered ring, each of the positions/atoms “4” and “5” will be deemed opposite to the position/atom at position “1” in Ring A.

When the Aliphatic Ring A is an 8-membered ring, positions “4”, “5” and “6” (and in particular position “5”) are deemed to be opposite to position “1” in the ring; and when the Aliphatic Ring A is an 9-membered ring, positions “5” and “6” are deemed to be opposite to position “1” in Ring A; and when the Aliphatic Ring A is a 10-membered ring, positions “5”, “6” and “7” (and in particular position “6”) are deemed to be opposite to position “1” in Ring A (and as further described herein, the Aromatic Ring B will be linked to such an 8-, 9- or 10-membered Aliphatic Ring A at one of the positions/atoms in Ring A that is opposite to the atom in position “1”, i.e. the atom/position to which the Aromatic Ring System C is linked). Based on the disclosure herein, it will also be clear to the skilled person which positions/atoms should be considered “opposite” to position “1” in Ring A when Ring A is a bridged ring system or a spiro ring system, as essentially the same considerations will apply.

From the further description herein, it will also be clear to the skilled person that the compounds of the invention will generally, and preferably, comprise a second aromatic ring system (which is different from the Aromatic Ring B) which second aromatic ring system is covalently linked either directly or via an alkylene linking group and in particular a methylene linking group (both as defined herein, and which as mentioned herein may also be a carbonyl group) to the position/atom on the Aliphatic Ring A that is indicated as “1” in the above formula (i.e. the position/atom “opposite to” the position/atom to which the Aromatic Ring B is linked). Generally, but again for the sake of convenience only and without limiting the scope of the present disclosure or claims, said second aromatic ring system (which is as further described herein) will also be referred to herein as “Aromatic Ring System C” or “Ring System C” and denoted as/by “[C]” and the position/atom on the Aliphatic Ring A to which said Aromatic Ring System C is linked will be considered as position “1” of the aliphatic ring A for the purposes of applying the numbering system that is schematically shown in the above Scheme A and further explained in the preceding paragraphs.

Thus, generally, the compounds of the invention can schematically be represented as having the following overall structure:

[Aromatic Ring System C]−[Aliphatic Ring A]−[Aromatic Ring B] Scheme B:

in which:
the Aromatic Ring System C, the Aliphatic Ring A and the Aromatic Ring B are each as further described herein;
   and:
the Aromatic Ring System C is either directly covalently bound to the Aliphatic Ring A or linked to the Aliphatic Ring A via an alkylene linking group (as defined herein, and which as mentioned herein may also be a carbonyl group), and in particular a methylene linking group (as defined herein);
   and:
the Aliphatic Ring A is either directly covalently bound to the Aromatic Ring B or linked to the Aromatic Ring B via an alkylene linking group (as defined herein, and which as mentioned herein may also be a carbonyl group), and in particular a methylene linking group (as defined herein);
   and:
the Aromatic Ring System C and the Aromatic Ring B are linked to the Aliphatic Ring A at ring atoms within the Aliphatic Ring A that are opposite to each other (as further defined herein).

In particular, the compounds of the invention can schematically be represented as having the following overall structure set out in the above Scheme B, in which:
the Aromatic Ring System C is a ring system that comprises one or more rings, in which at least one of the rings is an aromatic ring (and up to all of the rings can be aromatic rings), which ring system is as further described herein (and may optionally also be suitably substituted, as further described herein);
   and:
the Aliphatic Ring A has the overall structure that is as schematically represented by the following Scheme C:

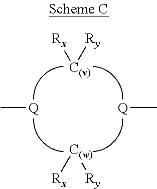

in which each C is a carbon atom, each atom Q is independently a carbon atom or a nitrogen atom (with preferably at least one of the atoms Q being a nitrogen atom and more preferably both atoms Q being nitrogen atoms), v and w are as further defined herein, and each of the Rₓ groups present and each R_{y} groups present are independently as further defined herein (and in which one of Rₓ or R_{y} linked to the C₍ᵥ₎ carbon chain and one of the Rₓ or R_{y} linked to the C_{(w)} carbon chain may be such that, taken together, they form an alkylene bridge or a covalent bond, such that the resulting structure is a bridged ring system or a fused ring system, respectively, both as further described herein); and more in particular an overall structure that is as schematically represented by the following Scheme D:

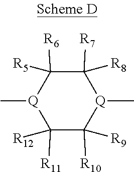

in which each atom Q is independently a carbon atom or a nitrogen atom (with preferably at least one of the atoms Q being a nitrogen atom and more preferably both atoms Q being nitrogen atoms), and each of R₅ to R₁₂ are independently as further defined herein (and in which one of R₅ or R₈ and one of R₉ or R₁₂ may be such that, taken together, they form an alkylene bridge or a covalent bond, such that the resulting structure is a bridged ring system or a fused ring system, respectively, both as further described herein); or in which alternatively the Aliphatic Ring A is a ring system of one of Formulae XCIX or C:

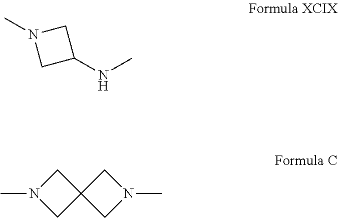

in which said ring system of Formula XCIX or Formula C may optionally be suitably substituted with one or more suitable substituents that, when present, are each preferably independently chosen from the group consisting of hydrogen, methyl, ethyl, halogen (in particular fluor (F)), CF₃ and isopropyl;
   and:
the Aromatic Ring B has an overall structure that is as schematically represented by the following Scheme E:

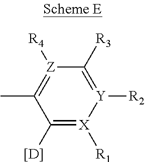

in which [D] represents the Acidic Substituent [D] (which is as further described herein), and each of X, Y and Z and each of R₁, R₂, R₃ and R₄ (when present) are as further described herein; and in particular an overall structure that is as schematically represented by the following Scheme F:

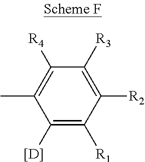

in which [D] represents the Acidic Substituent [D] (which is as further described herein), and each R₁, R₂, R₃ and R₄ are as further described herein; and more in particular an is overall structure that is as schematically represented by the following Scheme G:

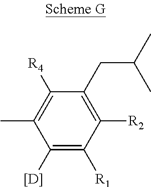

in which [D] represents the Acidic Substituent [D] (which is as further described herein), and each R₁, R₂ and R₄ are as further described herein;
   and:
the Aromatic Ring System C is either directly covalently bound to the Aliphatic Ring A or linked to the Aliphatic Ring A via an alkylene linking group (as defined herein, and which as mentioned herein may also be a carbonyl group), and in particular a methylene linking group (as defined herein);
   and
the Aliphatic Ring A is either directly covalently bound to the Aromatic Ring B or linked to the Aromatic Ring B via an alkylene linking group (as defined herein, and which as mentioned herein may also be a carbonyl group), and in particular a methylene linking group (as defined herein);
   and:
the Aromatic Ring System C and the Aromatic Ring B are linked to the Aliphatic Ring A at ring atoms within the Aliphatic Ring A that are opposite to each other (as further defined herein).

It should also be understood that, as will be clear to the skilled person from the further disclosure herein, if a preferred aspect is cited or described herein for one of the Aromatic Ring System C, the Aliphatic Ring A, the Aromatic Ring B and/or the Substituent D and/or any substituent(s) or combination of substituents, that said preferred aspect is preferably suitably combined with aspects that are cited or described herein as being preferred for the others of the Aromatic Ring System C, the Aliphatic Ring A, the Aromatic Ring B, the Substituent D and/or for any other substituent(s) or combination of substituents (and that the same will apply mutatis mutandis to aspects that cited or described herein as “more preferred”, “particularly preferred” and “most preferred” for each of the Aromatic Ring System C, the Aliphatic Ring A, the Aromatic Ring B, the Substituent D and/or said substituent(s) or combination of substituents).

Again, each of the Aromatic Ring System C, the Aliphatic Ring A, the Aromatic Ring B and the acidic Substituent D, as well as each of the alkylene linking groups C₍ₘ₎R_{A}R_{B} and C₍ₙ₎R_{C}R_{D} (when present) and the substituents R₁ to R₁₂ (when present) can generally be as further described herein, and are preferably each according to the preferred aspects that are described for each of them herein.

Thus, as further described herein, in a first specific but non-limiting aspect, the compounds of the invention will have the following structure (Formula I):

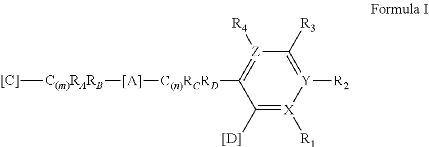

in which:
the aromatic ring system denoted by [C], the acidic substituent denoted by [D], each of X, Y and Z, each of R₁, R₂, R₃ and R₄ (when present), each of R_{A}, R_{B}, R_{C} and R_{D}, and m and n are each as further defined herein (and are in particular in accordance to preferred aspects that are described herein for each of these);
   and in which:
[A] represents a ring system that is as schematically represented by the following Scheme C:

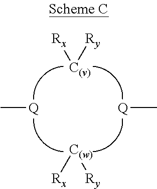

in which each atom Q, and each group Rₓ and R_{y} and each of v and w are as further described herein; and in particular a ring system that is as schematically represented by the following Scheme D:

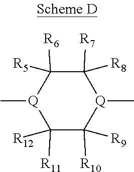

in which each Q and each of R₅ to R₁₂ are as further described herein; or alternatively [A] is a ring system of one of Formulae XCIX or C:

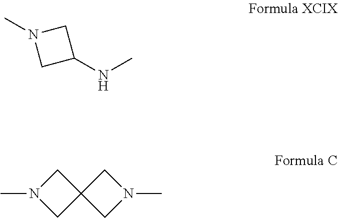

in which said ring system of Formula XCIX or Formula C may optionally be suitably substituted with one or more suitable substituents that, when present, are each preferably independently chosen from the group consisting of hydrogen, methyl, ethyl, halogen (in particular fluor (F)), CF₃ and isopropyl.

According to a more specific but non-limiting aspect, the compounds of the invention will have the following structure (Formula II):

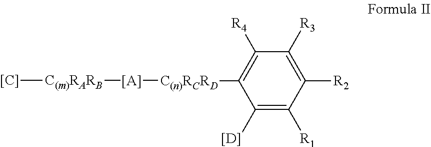

in which:
the aromatic ring system denoted by [C], the acidic substituent denoted by [D], each of R₁, R₂, R₃ and R₄, each of R_{A}, R_{B}, R_{C} and R_{D}, and m and n are each as further defined herein (and are in particular in accordance to preferred aspects that are described herein for each of these);
   and in which:
[A] represents a ring system that is as schematically represented by the following Scheme C:

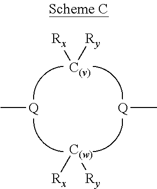

in which each atom Q, and each group Rₓ and R_{y} and each of v and w are as further described herein; and in particular a ring system that is as schematically represented by the following Scheme D:

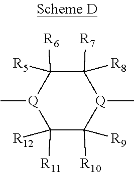

in which each Q and each of Rₛ to R₁₂ are as further described herein; or alternatively [A] is a ring system of one of Formulae XCIX or C:

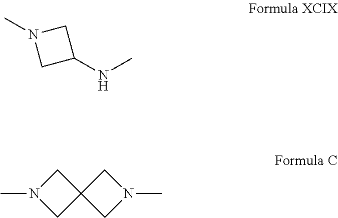

in which said ring system of Formula XCIX or Formula C may optionally be suitably substituted with one or more suitable substituents that, when present, are each preferably independently chosen from the group consisting of hydrogen, methyl, ethyl, halogen (in particular fluor (F)), CF₃ and isopropyl.

According to a more specific but non-limiting aspect, the compounds of the invention will have the following structure (Formula III):

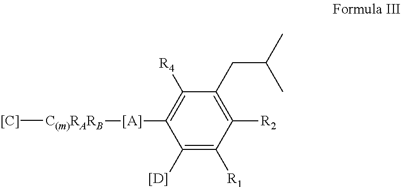

in which:
the aromatic ring system denoted by [C], the acidic substituent denoted by [D], each of R₁, R₂ and R₄, each of R_{A} and R_{B}, and m are each as further defined herein (and are in particular in accordance to preferred aspects that are described herein for each of these);
   and in which:
[A] represents a ring system that is as schematically represented by the following Scheme C:

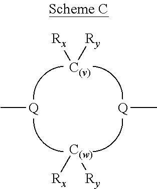

in which each atom Q, and each group Rₓ and R_{y} and each of v and w are as further described herein; and in particular a ring system that is as schematically represented by the following Scheme D:

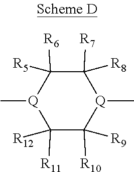

in which each Q and each of R₅ to R₁₂ are as further described herein; or alternatively [A] is a ring system of one of Formulae XCIX or C:

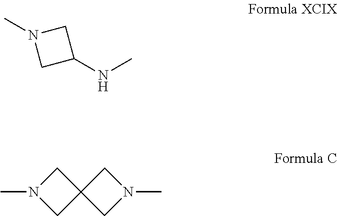

in which said ring system of Formula XCIX or Formula C may optionally be suitably substituted with one or more suitable substituents that, when present, are each preferably independently chosen from the group consisting of hydrogen, methyl, ethyl, halogen (in particular fluor (F)), CF₃ and isopropyl.

As further described herein, according to a preferred but non-limiting aspect of the invention, the compounds of the invention will have the following structure (Formula IV):

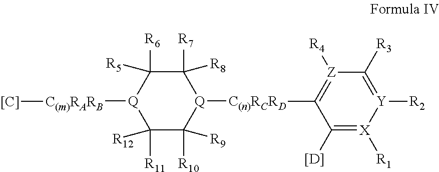

in which the aromatic ring system denoted by [C], the acidic substituent denoted by [D], each of X, Y and Z, each of R₁, R₂, R₃ and R₄ (when present), each of R₅, R₆, R₇, R₈, R₉, R₁₀, R₁₁ and R₁₂, each of R_{A}, R_{B}, R_{C} and R_{D}, each Q, and m and n are each as further defined herein (and are in particular in accordance to preferred aspects that are described herein for each of these).

As also described herein, according to a more preferred but non-limiting aspect of the invention, the compounds of the invention will have the following structure (Formula V):

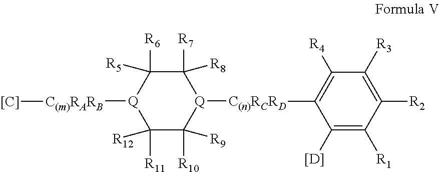

in which the aromatic ring system denoted by [C], the acidic substituent denoted by [D], each of R₁, R₂, R₃ and R₄, each of R₅, R₆, R₇, R₈, R₉, R₁₁, R₁₂ and R₁₂, each of R_{A}, R_{B}, R_{C} and R_{D}, each Q, and m and n are each as further defined herein (and are in particular in accordance to preferred aspects that are described herein for each of these).

As also described herein, according to a particularly preferred but non-limiting aspect of the invention, the compounds of the invention will have the following structure (Formula VI):

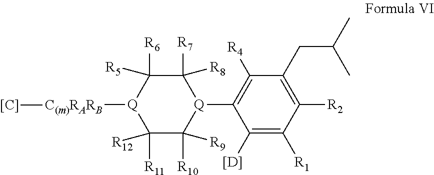

in which the aromatic ring system denoted by [C], the acidic substituent denoted by [D], each of R₁, R₂ and R₄, each of R₅, R₆, R₇, R₈, R₉, R₁₀, R₁₁ and R₁₂, each of R_{A} and R_{B}, each Q, and m are each as further defined herein (and are in particular in accordance to preferred aspects that are described herein for each of these).

In a specific but non-limiting aspect of the invention, in the compounds of the invention (and in particular in compounds of the invention of Formula I, Formula II, Formula III, Formula IV, Formula V and/or Formula VI):
the group R₁ is preferably chosen from the group consisting of H, halogen (F, Cl, Br or I, and preferably F or Cl), C₁-C₈ alkyl, C₃ or C₄ cycloalkyl, CF₃, C₁-C₈ alkoxy, amine (—NH₂) or C₁-C₂ substituted amine (e.g. dimethylamine or diethylamine) and cyano.

In another specific but non-limiting aspect of the invention, in the compounds of the invention (and in particular in compounds of the invention of Formula I, Formula II, Formula III, Formula IV, Formula V and/or Formula VI):
the group R₂ is preferably chosen from the group consisting of H, halogen (F, Cl, Br or I, and preferably F or Cl), C₁-C₈ alkyl, C₁-C₈ alkoxy, cycloalkyl, amine (—NH₂) or C₁-C₂ substituted amine (e.g. dimethylamine or diethylamine).

In another specific but non-limiting aspect of the invention, in the compounds of the invention (and in particular in compounds of the invention of Formula I, Formula II, Formula IV and/or Formula V):
the group R₃ is preferably chosen from the group consisting of H, halogen (F, Cl, Br or I, and preferably F or Cl), C₁₋₈ alkyl (e.g. sec-butyl and in particular iso-butyl), including fluor-substituted methyl groups (e.g. trifluoromethyl and CHF₂), C₁-C₈ alkoxy, —O—CF₃, methoxyethyloxy (—O—(CH₂)₂O—CH₃) or difluoroethoxy (—O—CH₂—CHF₂), cycloalkyl (e.g. cyclopropoxy, cyclobutoxy or cyclopentoxy), —CH₂— cycloalkyl, —O—CH₂-cycloalkyl, —O-cycloalkyl, —NH-cycloalkyl, —N(C₁-C₃)-cycloalkyl, —NH-heteroalkyl, —N(C₁-C₃)-heteroalkyl, heterocyclyl (e.g. oxirane), —CH₂-heterocyclyl, —O—CH₂-heterocyclyl (e.g. —O—CH₂-oxirane), —O-heterocyclyl, —NH-heterocyclyl, —N(C₁-C₃)-heterocyclyl, vinyl or methyl-substituted vinyl (e.g. —CH═CHCH₃, —CH═C(CH₃)₂ or —CH═CH₂), or allyl or methyl-substituted allyl (e.g. —CH₂CH═CH₂), isobutenyl or methyl substituted isobutenyl (e.g. ═C(CH₃)₂) and cyano.

In a particularly preferred aspect of the invention, the group R₃ is isobutyl (i.e. as in the compounds of the invention of Formula III and/or Formula VI).

In a more specific but non-limiting aspect of the invention, in the compounds of the invention (and in particular in compounds of the invention of Formula I, Formula II, Formula IV and/or Formula V):
the group R₁ is preferably chosen from the group consisting of H, halogen (F, Cl, Br or I, and preferably F or Cl), C₁-C₈ alkyl, C₃ or C₄ cycloalkyl, CF₃, C₁-C₈ alkoxy, amine (—NH₂) or C₁-C₂ substituted amine (e.g. dimethylamine or diethylamine) and cyano; and/or
the group R₂ is preferably chosen from the group consisting of H, halogen (F, Cl, Br or I, and preferably F or Cl), C₁-C₈ alkyl, C₁-C₈ alkoxy, cycloalkyl, amine (—NH₂) or C₁-C₂ substituted amine (e.g. dimethylamine or diethylamine); and/or
the group R₃ is preferably chosen from the group consisting of H, halogen (F, Cl, Br or I, and preferably F or Cl), C₁₋₈ alkyl (e.g. sec-butyl and in particular iso-butyl), including fluor-substituted methyl groups (e.g. trifluoromethyl and CHF₂) C₁-C₈ alkoxy, —O—CF₃, methoxyethyloxy (—O—(CH₂)₂O—CH₃) or difluoroethoxy (—O—CH₂—CHF₂), cycloalkyl (e.g. cyclopropoxy, cyclobutoxy or cyclopentoxy), —CH₂— cycloalkyl, —O—CH₂-cycloalkyl, —O-cycloalkyl, —NH-cycloalkyl, —N(C₁-C₃)-cycloalkyl, —NH-heteroalkyl, —N(C₁-C₃)-heteroalkyl, heterocyclyl (e.g. oxirane), —CH₂-heterocyclyl, —O—CH₂-heterocyclyl (e.g. —O—CH₂-oxirane), —O-heterocyclyl, —NH-heterocyclyl, —N(C₁-C₃)-heterocyclyl, vinyl or methyl-substituted vinyl (e.g. —CH═CHCH₃, —CH═C(CH₃)₂ or —CH═CH₂), or allyl or methyl-substituted allyl (e.g. —CH₂CH═CH₂), isobutenyl or methyl substituted isobutenyl (e.g. ═C(CH₃)₂) and
cyano; and is most preferably isobutyl (i.e. as in the compounds of the invention of Formula III and/or Formula VI).

In particular, according to this last aspect in the compounds of the invention (and in particular in compounds of the invention of Formula I, Formula II, Formula IV and/or Formula V):
the group R₁ is preferably chosen from the group consisting of H, halogen (F, Cl, Br or I, and preferably F or Cl), C₁-C₈ alkyl, C₃ or C₄ cycloalkyl, CF₃, C₁-C₈ alkoxy, amine (—NH₂) or C₁-C₂ substituted amine (e.g., dimethylamine or diethylamine) and cyano; and
the group R₂ is preferably chosen from the group consisting of H, halogen (F, Cl, Br or I, and preferably F or Cl), C₁-C₈ alkyl, C₁-C₈ alkoxy, cycloalkyl, amine (—NH₂) or C₁-C₂ substituted amine (e.g. dimethylamine or diethylamine); and
the group R₃ is preferably chosen from the group consisting of H, halogen (F, Cl, Br or I, and preferably F or Cl), C₁₋ₛ alkyl (e.g. sec-butyl and in particular iso-butyl), including fluor-substituted methyl groups (e.g. trifluoromethyl and CHF₂), C₁-C₈ alkoxy, —O—CF₃, methoxyethyloxy (—O—(CH₂)₂O—CH₃) or difluoroethoxy (—O—CH₂—CHF₂), cycloalkyl (e.g. cyclopropoxy, cyclobutoxy or cyclopentoxy), —CH₂-cycloalkyl, —O—CH₂-cycloalkyl, —O-cycloalkyl, —NH-cycloalkyl, —N(C₁-C₃)-cycloalkyl, —NH— heteroalkyl, —N(C₁-C₃)-heteroalkyl, heterocyclyl (e.g. oxirane), —CH₂-heterocyclyl, —O—CH₂-heterocyclyl (e.g. —O—CH₂-oxirane), —O-heterocyclyl, —NH-heterocyclyl, —N(C₁-C₃)-heterocyclyl, vinyl or methyl-substituted vinyl (e.g. —CH═CHCH₃, —CH═C(CH₃)₂ or —CH═CH₂), or allyl or methyl-substituted allyl (e.g. —CH₂CH═CH₂), isobutenyl or methyl substituted isobutenyl (e.g. ═C(CH₃)₂) and cyano; and is most preferably isobutyl (i.e. as in the compounds of the invention of Formula III and/or Formula VI).

In the compounds of the invention as described herein, whenever mention is made of an atom that is indicated by the letter Q (e.g. as in the compounds of Formulas IV, V and VI), each such atom Q can independently be a carbon atom or a nitrogen atom, preferably such that, when two such atoms Q are present in an aliphatic ring A as described herein, at least one such atom Q is a nitrogen atom and more preferably such that both such atoms Q in said aliphatic ring A are each a nitrogen atom.

In the compounds of the invention as described herein, when a group R₄ is present (e.g. as in the compounds described by Formulas I, II, III, IV, V and VI), said group R₄, when present, is preferably chosen from the group consisting of H or halogen (F, Cl, Br or I, and preferably F or Cl).

In the compounds of the invention as described herein, when a group R₅, R₆, R₇, R₈, R₉, R₁₀, R₁₁ and/or R₁₂ is present (e.g. as in the compounds described by Formulas IV, V and VI), each such group R₅, R₆, R₇, R₈, R₉, R₁₀, R₁₁ and/or R₁₂, when present, is preferably independently chosen from the group consisting of hydrogen, methyl, ethyl, fluor (F), CF₃ or isopropyl.

In the compounds of the invention as described herein, when a group R_{A}, R_{B}, R_{C}, and/or R_{D} is present (e.g. as in the compounds described by Formulas I, II, III, IV, V and VI, it being understood that the compounds of Formulas III and IV will not contain a group R_{C} or R_{D}), each such group R_{A}, R_{B}, R_{C} and/or R_{D}, when present, is independently chosen from hydrogen, methyl and/or trifluoromethyl, or where R_{A}+R_{B}, when present, together with the carbon atom to which they are bound form a carbonyl (C═O) group; and/or R_{C}+R_{D}, when present, together with the carbon atom to which they are bound form a carbonyl (C═O) group (in other words, in which R_{A}+R_{B} together are replaced by a single oxygen atom so as to form a carbonyl group with the carbon atom to which said oxygen atom is bound, and/or in which R_{C}+R_{D}, when present, together are replaced by a single oxygen atom so as to form a carbonyl group with the carbon atom to which said oxygen atom is bound), and are preferably each a hydrogen atom.

Generally, the compounds of the invention will have an affinity for AT2R (as measured according to the protocol set out in Example 2 below) that is better than 1.0×10⁻⁵ M (i.e. better than 10 micromolar), preferably better than 1.0×10⁻⁶M (i.e. better than 1 micromolar), more preferably better than 1.0×10⁻⁷ M (i.e. better than 0.1 micromolar), even more preferably better than 1.0×10⁻⁸ M (i.e. better than 10 nanomolar) [In this respect, it should be noted that: (i) in the present description and in accordance with generally accepted scientific practice, 10⁻⁵ will also be written as “E-05”, 10⁻⁶ will also be written as “E-06”, etc.; and that: (ii) by way of illustration, for the purposes of the present description and claims, an affinity of—for example—1.0×10⁻⁸ M (10 nanomolar) is considered “better” than an affinity of—for example—1.0×10⁻⁶ M (1 micromolar) and similarly an affinity of—for example—5 nanomolar is considered better than an affinity of—for example—10 nanomolar.]

Thus, in further specific but non-limiting aspects, the invention relates to:
a compound of the invention (as further described herein) that has an affinity for AT2R (measured according to the protocol set out in Example 2 below) that is better than 10 micromolar, preferably better than 1 micromolar, more preferably better than 0.1 micromolar, even more preferably better than 10 nanomolar;
   and in particular:
a compound of Formula I (as further described herein) that has an affinity for AT2R (measured according to the protocol set out in Example 2 below) that is better than 10 micromolar, preferably better than 1 micromolar, more preferably better than 0.1 micromolar, even more preferably better than 10 nanomolar;
   and more in particular:
a compound of Formula IV (as further described herein) that has an affinity for AT2R (measured according to the protocol set out in Example 2 below) that is better than 10 micromolar, preferably better than 1 micromolar, more preferably better than 0.1 micromolar, even more preferably better than 10 nanomolar;
   and even more in particular:
a compound of Formula II (as further described herein) that has an affinity for AT2R (measured according to the protocol set out in Example 2 below) that is better than 10 micromolar, preferably better than 1 micromolar, more preferably better than 0.1 micromolar, even more preferably better than 10 nanomolar;
   and even more in particular:
a compound of Formula V (as further described herein) that has an affinity for AT2R (measured according to the protocol set out in Example 2 below) that is better than 10 micromolar, preferably better than 1 micromolar, more preferably better than 0.1 micromolar, even more preferably better than 10 nanomolar;
   and even more in particular:
a compound of Formula III (as further described herein) that has an affinity for AT2R (measured according to the protocol set out in Example 2 below) that is better than 10 micromolar, preferably better than 1 micromolar, more preferably better than 0.1 micromolar, even more preferably better than 10 nanomolar;
   and even more in particular:
a compound of Formula VI (as further described herein) that has an affinity for AT2R (measured according to the protocol set out in Example 2 below) that is better than 10 micromolar, preferably better than 1 micromolar, more preferably better than 0.1 micromolar, even more preferably better than 10 nanomolar.

In further specific but non-limiting aspect, the invention relates to a compound of the invention (as further described herein) in which: (i) the Aromatic Ring System [C], the Aliphatic Ring [A], the Aromatic Ring [B] and the acidic substituent [D] are each as further described herein; and (ii) the substituent(s) that are present in such a compound of the invention (in which each such substituent can independently be as further described herein for the particular substituent involved) as well as the particular combination of such substituents that is present in said compound of the invention; and (iii) m and n (which as described herein can each independently be 0 or 1), are each such that (and are in combination such that) said compound of the invention has an affinity for AT2R (measured according to the protocol set out in Example 2 below) that is better than 10 micromolar, preferably better than 1 micromolar, more preferably better than 0.1 micromolar, even more preferably better than 10 nanomolar. In particular, the invention relates to such a compound of the invention in which each of the substituents R₁, R₂, R₃ and R₄ on the aromatic ring [B](when such substituent(s) are present on the aromatic ring [B], in accordance with the further definitions given herein) as well as the particular combination of such substituents R₁, R₂, R₃ and R₄ that is present on the aromatic ring [B], are each such that (and are in combination such that) said compound of the invention has an affinity for AT2R (measured according to the protocol set out in Example 2 below) that is better than 10 micromolar, preferably better than 1 micromolar, more preferably better than 0.1 micromolar, even more preferably better than 10 nanomolar.

In further specific but non-limiting aspects, the invention relates to:
a compound of Formula I (as further described herein) in which: (i) the Aromatic Ring System [C], the Aliphatic Ring [A] and the acidic substituent [D] are each as further described herein; and (ii) each of X, Y and Z, as well as the particular combination of the atoms X, Y and Z that is present in said compound of Formula I; and (iii) each of the substituents R₁, R₂, R₃ and R₄ (when present) as well as the particular combination of such substituents R₁, R₂, R₃ and R₄ that is present in said compound of Formula I; and (iv) m and n (which as described herein can each independently be 0 or 1); and (v) each of the substituents R_{A}, R_{B}, R_{C} and R_{D} (when present) as well as the particular combination of such substituents R_{A}, R_{B}, R_{C} and R_{D} that is present in said compound of Formula I; and (vi) any further substituents that are present in such a compound of Formula I (e.g. on the aromatic ring system [C] and/or on the aliphatic ring system [A], as further described herein) as well as the particular combination of such substituents that is present in said compound of Formula I, are each such that (and are in combination such that) said compound of Formula I has an affinity for AT2R (measured according to the protocol set out in Example 2 below) that is better than 10 micromolar, preferably better than 1 micromolar, more preferably better than 0.1 micromolar, even more preferably better than 10 nanomolar;
   and more in particular:
a compound of Formula IV (as further described herein) in which: (i) the Aromatic Ring System [C] and the acidic substituent [D] are each as further described herein; and (ii) each atom Q (which as mentioned herein can independently be a carbon atom or a nitrogen atom, with preferably at least one Q being a nitrogen atom); and (iii) each of X, Y and Z, as well as the particular combination of the atoms X, Y and Z that is present in said compound of Formula IV; and (iv) each of the substituents R₁, R₂, R₃ and R₄ (when present) as well as the particular combination of such substituents R₁, R₂, R₃ and R₄ that is present in said compound of Formula IV; and (v) each of the substituents R₅ to R₁₂ (when present) as well as the particular combination of such substituents R₅ to R₁₂ that is present in said compound of Formula IV; and (vi) m and n (which as described herein can each independently be 0 or 1); and (vii) each of the substituents R_{A}, R_{B}, R_{C} and R_{D} (when present) as well as the particular combination of such substituents R_{A}, R_{B}, R_{C} and R_{D} that is present in said compound of Formula IV; and (viii) any further substituents that are present in such a compound of Formula IV (e.g. on the aromatic ring system [C]) as well as the particular combination of such substituents that is present in said compound of Formula IV, are each such that (and are in combination such that) said compound of Formula IV has an affinity for AT2R (measured according to the protocol set out in Example 2 below) that is better than 10 micromolar, preferably better than 1 micromolar, more preferably better than 0.1 micromolar, even more preferably better than 10 nanomolar;
   and even more in particular:
a compound of Formula II (as further described herein) in which: (i) the Aromatic Ring System [C], the Aliphatic Ring [A] and the acidic substituent [D] are each as further described herein; and (ii) each of the substituents R₁, R₂, R₃ and R₄ (when present) as well as the particular combination of such substituents R₁, R₂, R₃ and R₄ that is present in said compound of Formula II; and (iii) m and n (which as described herein can each independently be 0 or 1); and (iv) each of the substituents R_{A}, R_{B}, R_{C} and R_{D} (when present) as well as the particular combination of such substituents R_{A}, R_{B}, R_{C} and R_{D} that is present in said compound of Formula II; and (v) any further substituents that are present in such a compound of Formula II (e.g. on the aromatic ring system [C] and/or on the aliphatic ring system [A], as further described herein) as well as the particular combination of such substituents that is present in said compound of Formula II, are each such that (and are in combination such that) said compound of Formula II has an affinity for AT2R (measured according to the protocol set out in Example 2 below) that is better than 10 micromolar, preferably better than 1 micromolar, more preferably better than 0.1 micromolar, even more preferably better than 10 nanomolar;
   and even more in particular:
a compound of Formula V (as further described herein) in which: (i) the Aromatic Ring System [C] and the acidic substituent [D] are each as further described herein; and (ii) each atom Q (which as mentioned herein can independently be a carbon atom or a nitrogen atom, with preferably at least one Q being a nitrogen atom); and (iii) each of the substituents R₁, R₂, R₃ and R₄ (when present) as well as the particular combination of such substituents R₁, R₂, R₃ and R₄ that is present in said compound of Formula V; and (v) each of the substituents R₅ to R₁₂ (when present) as well as the particular combination of such substituents R₅ to R₁₂ that is present in said compound of Formula V; and (vi) m and n (which as described herein can each independently be 0 or 1); and (vi) each of the substituents R_{A}, R_{B}, R_{C} and R_{D} (when present) as well as the particular combination of such substituents R_{A}, R_{B}, R_{C} and R_{D} that is present in said compound of Formula V; and (viii) any further substituents that are present in such a compound of Formula V (e.g. on the aromatic ring system [C]) as well as the particular combination of such substituents that is present in said compound of Formula IV, are each such that (and are in combination such that) said compound of Formula IV has an affinity for AT2R (measured according to the protocol set out in Example 2 below) that is better than 10 micromolar, preferably better than 1 micromolar, more preferably better than 0.1 micromolar, even more preferably better than 10 nanomolar;
   and even more in particular:
a compound of Formula III (as further described herein) in which: (i) the Aromatic Ring System [C], the Aliphatic Ring [A] and the acidic substituent [D] are each as further described herein; and (ii) each of the substituents R₁, R₂ and R₄ (when present) as well as the particular combination of such substituents R₁, R₂ and R₄ that is present in said compound of Formula III; and (iii) m (which as described herein can be 0 or 1); and (iii) each of the substituents R_{A} and R_{B} (when present) as well as the particular combination of such substituents R_{A} and R_{B} that is present in said compound of Formula III; and (iv) any further substituents that are present in such a compound of Formula III (e.g. on the aromatic ring system [C] and/or on the aliphatic ring system [A], as further described herein) as well as the particular combination of such substituents that is present in said compound of Formula III, are each such that (and are in combination such that) said compound of Formula III has an affinity for AT2R (measured according to the protocol set out in Example 2 below) that is better than 10 micromolar, preferably better than 1 micromolar, more preferably better than 0.1 micromolar, even more preferably better than 10 nanomolar;
   and even more in particular:
a compound of Formula VI (as further described herein) in which: (i) the Aromatic Ring System [C] and the acidic substituent [D] are each as further described herein; and (ii) each atom Q (which as mentioned herein can independently be a carbon atom or a nitrogen atom, with preferably at least one Q being a nitrogen atom); and (iii) each of the substituents R₁, R₂ and R₄ (when present) as well as the particular combination of such substituents R₁, R₂ and R₄ that is present in said compound of Formula VI; and (iv) each of the substituents R₅ to R₁₂ (when present) as well as the particular combination of such substituents R₅ to R₁₂ that is present in said compound of Formula VI; and (v) m (which as described herein can be 0 or 1); and (vi) each of the substituents R_{A} and R_{B}(when present) as well as the particular combination of such substituents R_{A} and R_{B} that is present in said compound of Formula VI; and (vii) any further substituents that are present in such a compound of Formula VI (e.g. on the aromatic ring system [C]) as well as the particular combination of such substituents that is present in said compound of Formula VI, are each such that (and are in combination such that) said compound of Formula VI has an affinity for AT2R (measured according to the protocol set out in Example 2 below) that is better than 10 micromolar, preferably better than 1 micromolar, more preferably better than 0.1 micromolar, even more preferably better than 10 nanomolar.

For the purposes of each of the above aspects of the invention and the further aspects of the invention as described herein, it should be noted that a substituent can also be a hydrogen atom (i.e., meaning that the relevant position in the compound of the invention is not substituted) provided the definition of said substituent, the further description of the relevant compound and/or the context allows.

As further described herein, the Aromatic Ring System C can be a monocyclic, bicyclic or polycyclic ring system, provided that at least one of the rings which is present in the ring system is an aromatic ring. According to a preferred aspect, essentially all of the rings that are present in the Aromatic Ring System C, are aromatic rings (although this is not critical).

Generally, although it will become clear to the skilled person from the description herein that the exact nature or structure of the Aromatic Ring System C is not critical as long as the Aromatic Ring System C contains at least one aromatic ring, it is preferred that the Aromatic Ring System C is a monocyclic or bicyclic ring system.

It is also preferred (but not critical) that the Aromatic Ring System C is suitably linked to the Aliphatic Ring A (i.e., either directly or via an alkylene linking group as defined herein and in particular a methylene linking group, both as defined herein) at a ring atom that forms part of an aromatic ring that is present within the Aromatic Ring System C.

The ring atoms that make up the Aromatic Ring System C can suitably consist entirely out of carbon atoms, or alternatively the Aromatic Ring System C can suitably comprise one or more hetero-atoms (in which any hetero-atoms that are present are preferably each independently but suitably chosen from N, S or O). When the Aromatic Ring System C suitably contains at least one hetero-atom, each of the rings that make up the Aromatic Ring System C can suitably contain no hetero-atoms or suitably contain one or more hetero-atoms, provided that the total number of hetero-atoms in the entire Aromatic Ring System C is at least one.

As will be clear to the skilled person, when the Aromatic Ring System C contains at least one hetero-atom, the number of hetero-atoms in the Aromatic Ring System C will usually depend on the total number of rings in the Aromatic Ring System C and the number of atoms in each ring. Generally, when the Aromatic Ring System C contains at least one hetero-atom, a 5-membered ring that forms part of the Aromatic Ring System C may suitably comprise up to three (such as 0, 1, 2 or 3) hetero-atoms and a 6-membered ring that forms part of the Aromatic Ring System C may suitably comprise up to three (such as 0, 1, 2 or 3) hetero-atoms, provided that the total number of hetero-atoms in the entire Aromatic Ring System C is at least one. When the Aromatic Ring System C is a bicyclic or polycyclic ring system, it is possible for two rings within the ring system to share a nitrogen atom. Also, when the Aromatic Ring System C contains at least one hetero-atom and is a monocyclic ring, the total number of hetero-atoms in the Aromatic Ring System C can suitably be 1, 2 or 3. Also, generally, when the Aromatic Ring System C contains two or more hetero-atoms, said hetero-atoms can suitably be the same or different (with again, as mentioned herein, preferably at least one of said hetero-atoms being a nitrogen atom).

As can be seen from the various formula's given herein, the Aromatic Ring System C is linked to one of the atoms Q in the Aliphatic Ring A. When said atom Q to which the Aromatic Ring System C is linked is a nitrogen atom, then the Aromatic Ring System C is preferably linked (i.e. either directly or via an alkylene linking group as defined herein, and in particular a methylene linking group, both as defined herein) to the Aliphatic Ring A at a carbon atom that is present in the Aromatic Ring System C (which carbon atom preferably, as mentioned herein, forms part of an aromatic ring within the Aromatic Ring System C, although this is again not critical) and not via a nitrogen atom in the Aromatic Ring System C (i.e. when the Aromatic Ring System C contains at least one nitrogen atom), because it is assumed that the resulting compounds will generally not be sufficiently stable for pharmaceutical use (unless the Aromatic Ring System C is linked to the remainder of the molecule via a carbonyl linking group that is linked to such a nitrogen atom in the Aromatic Ring System C).

In a preferred but non-limiting aspect, which aspect is also illustrated by some of the non-limiting examples of compounds of the invention that are given in the Experimental Part below, the Aromatic Ring System C: (i) contains at least one hetero-atom (with preferably at least one of said hetero-atoms being a nitrogen atom); and (ii) is such that it is linked to the Aliphatic Ring A (i.e. either directly via a covalent bond or via an alkylene linking group as further described herein) at a carbon atom within the Aromatic Ring System C that forms part of an aromatic ring within the Aromatic Ring System C (which ring, in case the Aromatic Ring System C is a monocyclic ring, essentially forms the Aromatic Ring System C); and (iii) is further such that said aromatic ring to which the Aliphatic Ring A is linked contains (i.e. in addition to the carbon atom to which the Aliphatic Ring A is linked) at least one (such as 1 or 2) hetero-atoms chosen from N, S and O (with preferably at least one of said hetero-atoms being a nitrogen atom).

Examples of aromatic ring systems that can be present as the Aromatic Ring System C in the compounds of the invention will be clear to the skilled person based on the disclosure herein, and generally include the monocyclic, bicyclic and polycyclic aromatic ring systems referred to herein.

Some preferred but non-limiting examples of aromatic ring systems that can be present as the Aromatic Ring System C in the compounds of the invention will be clear to the skilled person based on the further disclosure herein and include:

An Unsubstituted or Suitably Substituted pyrazolyl Group (Such as a pyrazol-3-yl Group), for Example:
2,5-dimethylpyrazol-3-yl;
1-methylpyrazol-3-yl;
2-methylpyrazol-3-yl;
   An Unsubstituted or Substituted Imidazolyl Group (Such as an imidazol-2-yl Group or an imidazol-4-yl Group), for Example:
1-methylimidazol-2-yl;
1-methylimidazol-4-yl;
3-methylimidazol-4-yl;
2,3-dimethylimidazol-4-yl;
   An Unsubstituted or Substituted triazolyl group (such as a 1,2,4-triazol-3-yl Group), for Example:
4-methyl-1,2,4-triazol-3-yl;
A pyrazine group;
   An Unsubstituted or Substituted Pyrimidine Group (Such as a 2-pyrimidine or 4-pyrimidine Group), for Example:
2-pyrimidine;
4-[2-methyl]-pyrimidine;
   An Unsubstituted or Substituted Pyridazine Group (Such as a 3-pyridazine Group), for Example:
6-methyl-3-pyridazine;
5-methyl-3-pyridazine;
6-methoxy-3-pyridazine;
   An Unsubstituted or Substituted Pyridyl Group (Such as a 2-pyridyl Group), for Example:
4-chloro-2-pyridyl;
5-chloro-2-pyridyl;
3-chloro-2-pyridyl;
4-methoxy-2-pyridyl;
5-methoxy-2-pyridyl;
4-fluoro-2-pyridyl;
5-fluoro-2-pyridyl;
4-methoxy-3,5-dimethyl-2-pyridyl;
3,5-dimethyl-2-pyridyl;
3-methoxy-2-pyridyl;
5-methyl-2-pyridyl;
4-(trifluoromethyl)-2-pyridyl;
3-methyl-2-pyridyl;
4-methyl-2-pyridyl;
5-chloro-3-fluoro-2-pyridyl;
4-methoxy-3-methyl-2-pyridyl;
3-chloro-5-fluoro-2-pyridyl;
4,5-dimethoxy-2-pyridyl;
4-chloro-5-fluoro-2-pyridyl;
3,5-difluoro-2-pyridyl;

### An Unsubstituted or Substituted Indazole Group, for Example:

1-methyl-indazole;
   An Unsubstituted or Substituted imidazo[1,2-a]pyridine Group, for Example:
imidazo[1,2-a]pyridine;
   An Unsubstituted or Substituted quinoline Group, for Example:
2-quinoline;
   An Unsubstituted or Substituted quinazoline Group, for Example:
2-quinazoline;
   An Unsubstituted or Substituted quinoxaline Group, for Example:
2-methyl-3-quinoxaline;
   An Unsubstituted or Substituted Oxazole Group (Such as an 1,4-oxazole Group), for Example:
1,4-oxazole;

### An Unsubstituted or Substituted Isoxazole Group (Such as an Isoxazole Group), for Example:

5-methyl-isoxazole;
   An Unsubstituted or Substituted Oxadiazole Group (Such as an 1,2,4-oxadiazole Group), for Example:
1,2,4-oxadiazole;
3-methyl-1,2,4-oxadiazole;
5-methyl-1,2,4-oxadiazole;
5-methyl-1,3,4-oxadiazole;
   An Unsubstituted or Substituted Benzoxazole Group (Such as an 1,3-benzoxazole Group), for Example:
2-[1,3-benzoxazole];
   An Unsubstituted or Substituted oxazolopyridine Group (Such as an oxazolo[4,5-b]pyridine Group), for Example:
oxazolo[4,5-b]pyridine;
   An Unsubstituted or Substituted Thiazole Group (Such as a 2-thiazole Group or a 4-thiazole Group), for Example:
5-methyl-4-thiazole;
5-methyl-2-thiazole;
2-methyl-thiazole;
   An Unsubstituted or Substituted Benzothiazolyl Group (such as an 1,3-benzothiazol-2-yl group), for example:
1,3-benzothiazol-2-yl;
   An unsubstituted or substituted pyrimidinone group (such as an pyrimidin-4-one group), for example:
2-methyl-1H-pyrimidin-4-one;
6-methyl-1H-pyrimidin-4-one;
   An unsubstituted or substituted quinazolin-4-on group (such as a quinazolin-4-on group), for example:
quinazolin-4-on;
2-ethyl-3-quinazolin-4-one;
   An unsubstituted or substituted quinolinone group (such as a quinolin-2-on group), for example:
1-methyl-quinolin-2-one; An unsubstituted or substituted bicyclic pyrimidinone group, for example:
1-methyl-7H-pyrazolo[3,4-d]pyrimidin-4-one;
8-methyl-pyrido[1,2-a]pyrimidin-4-one;
1-methyl-pyrazolo[1,5-a]pyrimidin-7-one;
pyrido[1,2-a]pyrimidin-4-one;
6-methyl-pyrido[1,2-a]pyrimidin-4-one;
3-methyl-thiazolo[3,2-a]pyrimidin-5-one.

As will be clear to the skilled person based on the disclosure herein, the Aromatic Ring System C can also be suitably substituted (as defined herein) by one or more (such as 1, 2, 3 or 4) suitable substituents on one or more (such as 1, 2, 3 or 4) suitable positions/ring atoms of the Aromatic Ring System C. Suitable substituents will be clear to the skilled person and for example include the substituent(s) that are present on the Aromatic Ring System C in the compounds of the invention that are illustrated in the Experimental Part below, as well as other suitable substituents mentioned herein. In a specific but non-limiting embodiment of the invention, the Aromatic Ring System C does not carry a carboxylic acid (COOH) group.

As generally mentioned herein, the total number of suitable substituents on the Aromatic Ring System C is generally not critical and will usually depend on the size of the Aromatic Ring System C and the number of ring atoms in the Aromatic Ring System C. Usually, the total number of substituents will be:
when the Aromatic Ring System C is a monocyclic 5-membered ring: 0, 1, 2 or 3, and preferably 0, 1 or 2;
when the Aromatic Ring System C is a monocyclic 6-membered ring: 0, 1, 2 or 3, and preferably 0, 1 or 2;
when the Aromatic Ring System C is a bicyclic ring system: 0, 1, 2, 3, 4 or 5, and preferably 0, 1, 2 or 3;

According to a specific, but non-limiting aspect, each of the one or more substituents on the Aromatic Ring System C is each suitably and independently chosen from one or more of: methyl, ethyl, cyclopropyl, methoxy, trifluoromethyl, cyano/nitrile, a halogen atom (in particular fluorine or chlorine), a C₁-C₄ amine or an oxygen atom (i.e. so as to form, together with the carbon atom to which said oxygen is linked, a carbonyl group where said carbon atom is capable of forming such a carbonyl group).

In one non-limiting aspect of the invention, the Aromatic Ring System C is either:
a monocyclic 5- or 6-membered aromatic ring system, and preferably a monocyclic 5- or 6-membered aromatic ring system that comprises at least one nitrogen atom; or
a bicyclic aromatic ring system (as defined herein) in which each ring is a 5-membered and/or a 6-membered aromatic ring (i.e. such that the entire bicyclic ring system forms a conjugated planar ring system), and preferably a bicyclic aromatic ring system in which each ring is a 5-membered or 6-membered aromatic ring and in which the aromatic ring to which the Aliphatic Ring [A] is linked comprises at least one nitrogen atom.

As already generally mentioned herein for the Aromatic Ring System C, when the Aromatic Ring System C is a monocyclic or bicyclic aromatic ring system that is comprised of one or two 5- and/or 6-membered rings, respectively, it is preferably such that it suitably comprises at least one hetero-atom (with each such hetero-atom preferably being independently but suitably chosen from O, N or S, with preferably at least one of the hetero-atoms present being a nitrogen atom), with the total number of hetero-atoms in the Aromatic Ring System C preferably being 1 or 2 in case of a monocyclic ring system and preferably being 1, 2, 3 or 4 in case of a bicyclic ring system. Also, and again as exemplified by some of the non-limiting examples of compounds of the invention that are given in the Experimental Part below, such an Aromatic Ring System C that is comprised of one or two 5- and/or 6-membered rings, respectively, is preferably further such that the Aliphatic Ring A is linked (i.e. either directly via a covalent bond or via an alkylene linking group as further described herein) to a carbon atom within the Aromatic Ring System C, which carbon atom forms part of an aromatic ring within the Aromatic Ring System C (which ring, in case the Aromatic Ring System C is a monocyclic ring, essentially forms the Aromatic Ring System C), in which said aromatic ring contains, in addition to said one carbon atom to which the Aliphatic Ring A is linked, at least one (such as 1 or 2) hetero-atoms chosen from N, S and O (with preferably at least one of the hetero-atoms present being a nitrogen atom). Some non-limiting examples of aromatic ring systems that can be present in the compounds of the invention as the Aromatic Ring System C include the following (in which it should be understood that: (i) when, in the following Tables A, B, C and D, reference is made to the atom “A” or “Q” and to the groups R, R₁, R₂, R₃ and R₄, such atoms A and Q and such groups R, R₁, R₂, R₃ and R₄ are as defined in said Tables A to D; and (ii) that the definitions of the atoms A and Q and of the groups R, R₁, R₂, R₃ and R₄ that are given in the Tables A to D below only apply to the structures shown in said Tables and not to any other atoms, groups, structures or formulas set out in the present description):

TABLE A

| Formula | Structure |
|---|---|
| VII | 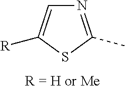 |
| VIII | 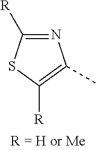 |
| IX | 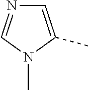 |
| X | 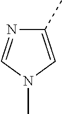 |
| XI | 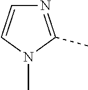 |
| XII | 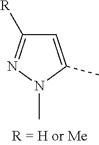 |
| XIII | 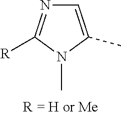 |
| XIV | 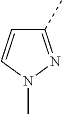 |
| XV | 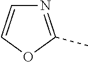 |
| XVI | 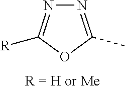 |
| XVII | 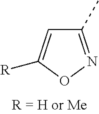 |
| XVIII | 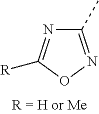 |
| XIX | 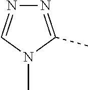 |
| XX | 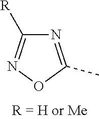 |
| XXI | 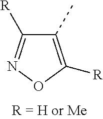 |
| XXII | 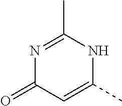 |
| XXIII | 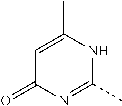 |
| XXIV | 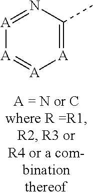 |
| XXV | 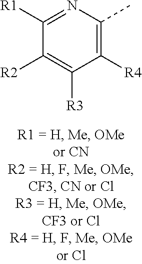 |
| XXVI | 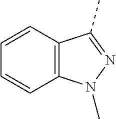 |
| XXVII | 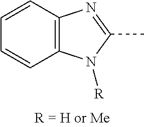 |
| XXVIII | 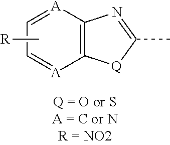 |
| XXIX | 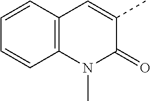 |
| XXX | 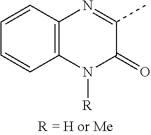 |
| XXXI | 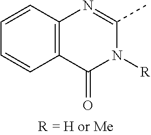 |
| XXXII | 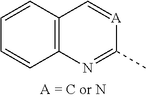 |
| XXXIII | 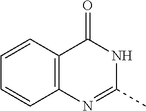 |
| XXXIV | 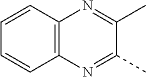 |
| XXXV | 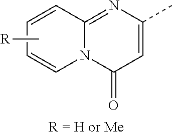 |
| XXXVI | 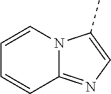 |
| XXXVII | 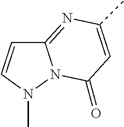 |
| XXXVIII | 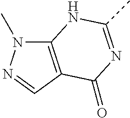 |
| XXXIX | 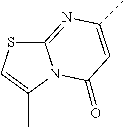 |

with some specific examples including the following ring systems:

TABLE B

| Formula | Structure |
|---|---|
| XL | 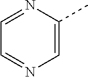 |
| XLI | 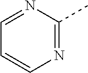 |
| XLII | 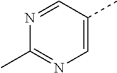 |
| XLIII | 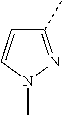 |
| XLIV | 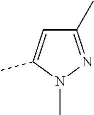 |
| XLV | 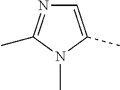 |
| XLVI | 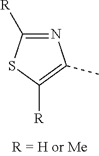 |
| XLVII | 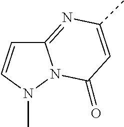 |
| XLVIII | 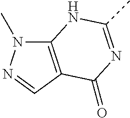 |
| XLIX | 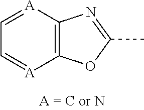 |
| L | 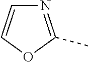 |
| LI | 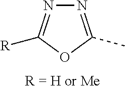 |
| LII | 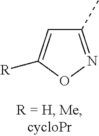 |
| LIII | 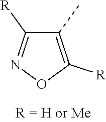 |
| LIV | 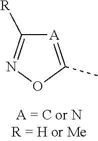 |
| LV | 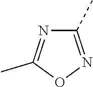 |
| LVI | 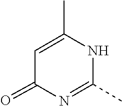 |
| LVII | 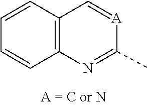 |
| LVIII | 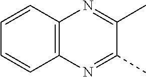 |
| — | — |

and with some preferred examples including the following ring systems:

TABLE C

| Formula | Structure |
|---|---|
| LIX | 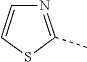 |
| LX | 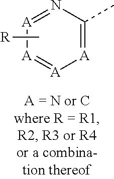 |
| LXI | 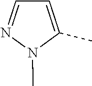 |
| LXII | 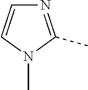 |
| LXIII | 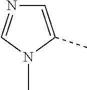 |
| LXIV | 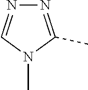 |
| LXV | 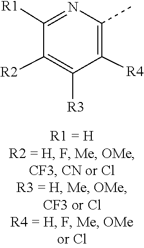 |
| LXVI | 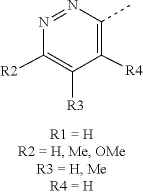 |
| LXVII | 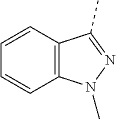 |
| LXVIII | 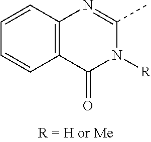 |
| LXIX | 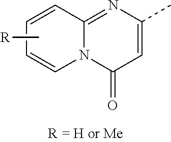 |
| LXX | 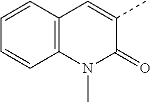 |
| LXXI | 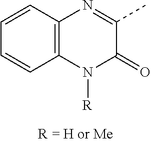 |
| LXXII | 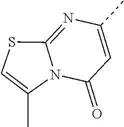 |

and with the following rings systems being particularly preferred:

TABLE D

| Formula | Structure |
|---|---|
| LXXIII | 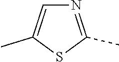 |
| LXXIV | 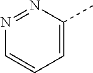 |
| LXXV | 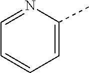 |
| LXXVI | 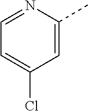 |
| LXXVII | 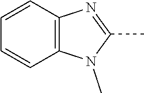 |
| LXXVIII | 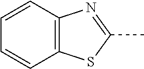 |
| LXXIX | 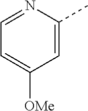 |
| LXXX | 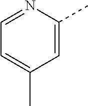 |
| LXXXI | 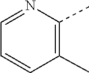 |
| LXXXII | 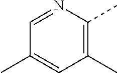 |
| LXXXIII | 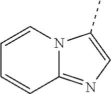 |
| — | — |

Thus, according to one non-limiting aspect of the invention, the Aromatic Ring System C is chosen from the group consisting of the aromatic rings/ring systems of Formulae VII to LXXXIII.

According to a preferred but non-limiting aspect of the invention, the Aromatic Ring System C is chosen from the group consisting of the aromatic rings/ring systems of Formulae LIX to LXXXIII.

According to a particularly preferred but non-limiting aspect of the invention, the Aromatic Ring System C is chosen from the group consisting of the aromatic rings/ring systems of Formulae LXXIII to LXXXIII.

Thus, in further aspects, the invention relates to:
a compound of the invention (as further described herein) in which the Aromatic Ring System C is chosen from the group consisting of the aromatic rings/ring systems of Formulae VII to LXXXIII, preferably from the group consisting of the aromatic rings/ring systems of Formulae LIX to LXXXIII, and more preferably from the group consisting of the aromatic rings/ring systems of Formulae LXXIII to LXXXIII;
   and in particular:
a compound of Formula I (as further described herein) in which the Aromatic Ring System C is chosen from the group consisting of the aromatic rings/ring systems of Formulae VII to LXXXIII, preferably from the group consisting of the aromatic rings/ring systems of Formulae LIX to LXXXIII, and more preferably from the group consisting of the aromatic rings/ring systems of Formulae LXXIII to LXXXIII;
   and more in particular:
a compound of Formula IV (as further described herein) in which the Aromatic Ring System C is chosen from the group consisting of the aromatic rings/ring systems of Formulae VII to LXXXIII, preferably from the group consisting of the aromatic rings/ring systems of Formulae LIX to LXXXIII, and more preferably from the group consisting of the aromatic rings/ring systems of Formulae LXXIII to LXXXIII;
   and even more in particular:
a compound of Formula II (as further described herein) in which the Aromatic Ring System C is chosen from the group consisting of the aromatic rings/ring systems of Formulae VII to LXXXIII, preferably from the group consisting of the aromatic rings/ring systems of Formulae LIX to LXXXIII, and more preferably from the group consisting of the aromatic rings/ring systems of Formulae LXXIII to LXXXIII;
   and even more in particular:
a compound of Formula V (as further described herein) in which the Aromatic Ring System C is chosen from the group consisting of the aromatic rings/ring systems of Formulae VII to LXXXIII, preferably from the group consisting of the aromatic rings/ring systems of Formulae LIX to LXXXIII, and more preferably from the group consisting of the aromatic rings/ring systems of Formulae LXXIII to LXXXIII;
   and even more in particular:
a compound of Formula III (as further described herein) in which the Aromatic Ring System C is chosen from the group consisting of the aromatic rings/ring systems of Formulae VII to LXXXIII, preferably from the group consisting of the aromatic rings/ring systems of Formulae LIX to LXXXIII, and more preferably from the group consisting of the aromatic rings/ring systems of Formulae LXXIII to LXXXIII;
   and even more in particular:
a compound of Formula VI (as further described herein) in which the Aromatic Ring System C is chosen from the group consisting of the aromatic rings/ring systems of Formulae VII to LXXXIII, preferably from the group consisting of the aromatic rings/ring systems of Formulae LIX to LXXXIII, and more preferably from the group consisting of the aromatic rings/ring systems of Formulae LXXIII to LXXXIII;
   in which such compound of the invention (i.e. a compound of the invention according to any of the preceding aspects) is preferably further such that such compound has an affinity for AT2R (measured according to the protocol set out in Example 2 below) that is better than 10 micromolar, preferably better than 1 micromolar, more preferably better than 0.1 micromolar, even more preferably better than 10 nanomolar (and for the remainder is as further described herein).

As generally described herein, for each of the aromatic rings/ring systems shown as Formulae VII to LXXXIII, respectively, it is not excluded that, if such a ring or ring system is present as the Aromatic Ring System C in a compound of the invention, such aromatic ring or ring system can suitably be substituted (or, in case of an aromatic ring of Formulae VII to LXXXIII that already carries one or more substitutions, suitably further substituted) with one or more (such as 1, 2 or 3) further substituents (in which suitable substituents will be clear to the skilled person and for example include the substituent(s) that are present on the Aromatic Ring System C in the compounds of the invention that are illustrated in the Experimental Part below, as well as other suitable substituents mentioned herein). However, for each of the aromatic rings/ring systems shown as Formulae VII to LXXXIII, it is generally preferred that these are present as the Aromatic Ring System C in a compound of the invention without any such (additional) substituents, in particular where such an aromatic ring or ring system is shown, in one of Formulae VII to LXXXIII, to already carry one or more substituents. Also, in a specific but non-limiting embodiment of the invention, the Aromatic Ring System C does not carry a carboxylic acid (COOH) group.

Thus, preferably, the aromatic ring or ring system that is present as the Aromatic Ring System C in a compound of the invention preferably consists of one of the aromatic rings/ring systems having the structure set out in Formulae VII to LXXXIII, more preferably of one of the aromatic rings/ring systems having the structure set out in Formulae LIX to LXXXIII, and most preferably of one of the aromatic rings/ring systems having the structure set out in Formulae LXXIII to LXXXIII.

Some specific but non-limiting examples of compounds of the invention (including such compounds of the invention that are particularly preferred) that contain an Aromatic Ring System C as described herein will become clear to the skilled person based on the disclosure herein and/or are as exemplified in the Experimental Part below.

As mentioned herein, the Aromatic Ring C is linked, either directly via a covalent bond but preferably via an alkylene linking group (as defined herein, and which as mentioned herein may also be a carbonyl group) and in particular a methylene linking group (as defined herein), to an aliphatic ring, which aliphatic ring is as further described herein and which, as mentioned, is also referred to as “Aliphatic Ring A” or “Ring A” and also denoted as/by “[A]”.

The Aliphatic Ring A is generally a 5-, 6-, 7-, 8-, 9- or 10-membered ring, and is preferably a 5-, 6- or 7-membered ring, and is most preferably a 6-membered ring, in which said ring is essentially comprised of carbon atoms and one or more (such as 1 or 2) hetero-atoms (said hetero-atoms, when present, preferably each being independently chosen from O, S and N, but most preferably being nitrogen atoms, as further described herein). It is also not excluded that the Aliphatic Ring A may also suitably contain one or more double bonds (provided that the presence of these bonds do not make the Aliphatic Ring A into an aromatic ring system), but preferably Ring A is fully saturated (i.e. without any double bonds in the ring) as it is expected that compounds that contain an unsaturated Ring A will usually not be sufficiently stable for pharmaceutical use.

The Aliphatic Ring A is preferably a monocyclic ring, but as further described herein may also be a bridged monocyclic ring that suitably comprises a suitable alkylene bridge (as defined herein) that comprises 1 or 2 carbon atoms, such that the Aliphatic Ring A forms part of a “bicyclo”-type structure that comprises between 7 and 12 atoms in total (including the alkylene bridge) and preferably comprises 7, 8 or 9 atoms in total (including the alkylene bridge). For example and without limitation, the Aliphatic Ring A may comprise a cyclohexane, piperidine or piperazine ring that suitably comprises a suitable alkylene bridge (as defined herein) that comprises 1 or 2 carbon atoms (for example, suitably bridging positions “2” and “6”, positions “2” and “5”, positions “3” and “6” or positions “3” and “5” in the Aliphatic Ring A, such as 3,8-diazabicyclo[3.2.1]octane and 2,5-diazabicyclo[2.2.1]heptane (see also Formulae LXXXV and XCII herein). In the Experimental Part below, compounds A-189 and A-190 provide some specific but non-limiting example of compounds of the invention that comprise a bridged Aliphatic Ring A. As also generally mentioned herein, aliphatic 5-, 6-, 7-, 8-, 9- and 10-membered aliphatic ring systems in which two of the ring atoms are suitably “bridged” by a covalent bond so as to form a system that comprises two fused rings that share said two “bridged” atoms (for example, as in the aliphatic ring system of Formula XCVIII and the aliphatic ring system that is present in Compounds A-232 and A-233) are also deemed to be bridged ring structures for the purposes of the present description and claims. Such a bridged ring structure or fused ring structure may optionally also be suitably substituted with one or more substituents that, when present, may each independently be chosen from the substituents mentioned herein for the groups Rₓ or R_{y} or R₅ to R₁₂.

The Aliphatic Ring A may also be a spiro-type structure (with any such spiro structure, preferably containing between 7 and 10 atoms in total), as for example exemplified by the spiro-type structure shown as Formula C below. Such a spiro-type structure may optionally also be suitably substituted with one or more substituents that, when present, may each independently be chosen from the substituents mentioned herein for the groups Rₓ or R_{y} and/or R₅ to R₁₂.

As further described herein, the Aliphatic Ring A is linked (either directly or via a alkylene linking group as further described herein) to the Aromatic Ring System C and also linked (again either directly or via an alkylene linking group as further described herein) to the Aromatic Ring B, preferably either such that the Aromatic Ring System C is linked to the Aliphatic Ring A via an alkylene linking group (with the Aromatic Ring B being directly linked to the Aliphatic Ring A) or such that the Aromatic Ring B is linked to the Aliphatic Ring A via an alkylene linking group (with the Aromatic Ring System C being directly linked to the Aliphatic Ring A), and more preferably such that the Aromatic Ring System C is linked to the Aliphatic Ring A via an alkylene linking group (with the Aromatic Ring B being directly linked to the Aliphatic Ring A). As described herein, when present, the alkylene linking group is preferably a methylene linking group (i.e. as represented in Formulae I to VI as “C₍ₘ₎R_{A}R_{B}” and “C₍ₙ₎R_{C}R_{D}”, respectively). As also mentioned herein, when such a linking group is present, it may also be a carbonyl group instead of a methylene group (i.e. with C₍ₘ₎R_{A}R_{B} or C₍ₙ₎R_{C}R_{D} being C═O).

Also, as further described herein, the Aromatic Ring System C and the Aromatic Ring B are most preferably linked to the Aliphatic Ring A at atoms in the Aliphatic Ring A that are opposite to each other (as defined herein) in said Aliphatic Ring A.

As also further described herein, the Aliphatic Ring A is preferably such that it contains at least one nitrogen atom, which nitrogen atom is linked (either directly or via a methylene linking group as further described herein) to either the Aromatic Ring System C or the Aromatic Ring B. More preferably, the Aliphatic Ring A is such that it contains two nitrogen atoms, with one of said nitrogen atoms being linked (either directly or via a methylene linking group as further described herein) to the Aromatic Ring System C and the other of said nitrogen atoms being linked (either directly or via a methylene linking group as further described herein) to the Aromatic Ring B. Again, when the Aliphatic Ring A contains one such nitrogen atom or two such nitrogen atoms, the ring atoms in Ring A to which the Aromatic Ring System C and the Aromatic Ring B, respectively, are linked are most preferably positioned opposite to each other (as defined herein) in the Aliphatic Ring A.

In a specific but non-limiting preferred aspect, an Aliphatic Ring A as described herein consists of carbon atoms and 0, 1 or 2 (and preferably 1 or 2 and more preferably 2) nitrogen atoms, in particular such that the Aliphatic Ring A contains a total of 5, 6 or 7 (and preferably 6) ring atoms (excluding any carbon atoms that are present in an alkylene bridge if an alkylene bridge is present).

Thus, generally, the Aliphatic Ring A can have an overall structure that can be schematically represented as follows (Scheme C):

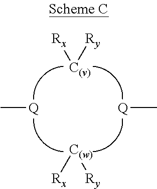

in which:
each C, when present, represents a carbon atom;
   and
each Q is independently a carbon atom or a nitrogen atom, preferably such that at least one atom Q is a nitrogen atom, and more preferably such that both atoms Q are nitrogen atoms;

and
v is an integer between 1 and 4, and w is an integer between 1 and 4, such that the sum of (v+w) is 3, 4, 5, 6, 7 or 8 (and preferably 3, 4 or 5, with v and w more preferably both being 2) and such that the difference (v−w) is either 1, 0 or −1;
   and
each carbon atom C carries a group Rₓ and a group R_{y}, in which each group Rₓ that is present can independently be hydrogen or a suitable substituent (in which said substituent is preferably chosen from hydrogen, methyl, ethyl, halogen (in particular fluor (F)), CF₃ and isopropyl) and each group R_{y} that is present can independently be hydrogen or a substituent chosen from a suitable substituent (in which said substituent is preferably chosen from hydrogen, methyl, ethyl, halogen (in particular fluor (F)), CF₃ and isopropyl).

As also described and exemplified herein, the Ring A can also be a ring that is suitably bridged by an alkylene bridge (as defined herein, but not shown in Scheme C) or a covalent bond (also not shown in Scheme C), in which one end of said alkylene bridge or covalent bond is linked to one of the carbon atoms that make up the carbon chain represented by C₍ᵥ₎ and the other end of said alkylene bridge or covalent bond is linked to one of the carbon atoms that make up the carbon chain represented by C_{(w)}. As will be clear to the skilled person, when Ring A is bridged by an alkylene bridge, one of the Rₓ or R_{y} on the carbon chain represented by C₍ᵥ₎ and one of the Rₓ or R_{y} on the carbon chain represented by C_{(w)} will be replaced by the alkylene bridge; and when Ring A is bridged by a covalent bond, one of the Rₓ or R_{y} on the carbon chain represented by C₍ᵥ₎ and one of the Rₓ or R_{y} on the carbon chain represented by C_{(w)} will be replaced by said covalent bond.

Most preferably, the Ring A is not substituted with an oxygen atom (i.e. so as to form a carbonyl group with the ring carbon atom to which said oxygen is bound).

As mentioned, Ring A can also be a spiro-type structure (with any such spiro structure, preferably containing between 7 and 10 atoms in total), as for example exemplified by the spiro-type structures shown as Formula C below. Where possible in view of the number of (remaining) covalent bonds that are available on each carbon atom that is present in such a spiro structure, each carbon atom in such a spiro-type structure may also suitably carry a group Rₓ and/or a group R_{y} as defined herein.

With reference to Scheme C and Scheme D, the Aliphatic Ring A is linked, at one of the atoms Q in the Aliphatic Ring A, to the Aromatic Ring System C and is also linked, at the other of the atoms Q in the Aliphatic Ring A, to the Aromatic Ring B. For this purpose, the two atoms Q are preferably at positions in the Aliphatic Ring A that are opposite to each other (as further defined herein). For example, when the Aliphatic Ring A is a 6-membered ring, the Aromatic Ring System C is preferably linked to the ring atom of the Aliphatic Ring A that is at position “1” and the Aromatic Ring B is preferably linked to the ring atom of the Aliphatic Ring A that is at position “4” (with the numbering of the positions/atoms in the Aliphatic Ring A being as described herein). Similarly, when the Aliphatic Ring A is a 7-membered ring, the Aromatic Ring System C is preferably linked to the ring atom of the Aliphatic Ring A that is at position “1” and the Aromatic Ring B is preferably linked to the ring atom of the Aliphatic Ring A that is at position “4” or at position “5” (with the numbering of the positions/atoms in the Aliphatic Ring A being as described herein).

As also further described herein, the Aliphatic Ring A may be directly linked to the Aromatic Ring System C (i.e. via a covalent bond between one of the atoms Q and a ring atom in the Aromatic Ring System C) or via an alkylene linking group (as defined herein) and in particular a methylene linking group (as defined herein) which links an atom Q in the Aliphatic Ring A to a ring atom in the Aromatic Ring System C (in which, as further described herein, said ring atom in the Aromatic Ring System C is preferably a carbon atom which is present within an aromatic ring in said Aromatic Ring System C, which aromatic ring preferably contains at least one hetero-atom and in particular at least one nitrogen atom). As also further described herein, the Aliphatic Ring A may be directly linked to the Aromatic Ring B (i.e. via a covalent bond between the other atom Q and a ring atom in the Aromatic Ring B) or via an alkylene linking group (as defined herein) and in particular a methylene linking group (as defined herein) which links the other atom Q in the Aliphatic Ring A to a ring atom in the Aromatic Ring B (in which, as further described herein, said ring atom in the Aromatic Ring B is in an ortho position relative to the Acidic Substituent D). In the above Formulae I to VI, this is represented by “—C₍ₘ₎R_{A}R_{B}—” and “—C₍ₙ₎R_{C}R_{D}—”, respectively, in which m can be 0 or 1 and n can be 0 or 1 (with m=0 or n=0, respectively, meaning that a direct covalent bond is present), so that the sum of m+n can be 2, 1 or 0. Most preferably, at least one such an alkylene linking group (as defined herein), and in particular at least one such methylene linking group (as defined herein), is present, meaning that either m=1 and n=0 or m=0 and n=1 with the sum of m+n being 1 or 0 (with m and n both being 0 or both being 1, meaning that the sum of m+n is either 0 or 2, respectively, being much less preferred). According to a particularly preferred aspect, m=1 and n=0 (as is the case in the structures of Formulae III and VI).

It will be clear to the skilled person that, according to a preferred aspect of the ring that is represented by Scheme C in which v is 2 and w is 2, the Aliphatic Ring A will be a 6-membered ring. In such a 6-membered ring, each carbon atom can optionally (and independently) be substituted with one or two suitable substituents, which are preferably and independently chosen from hydrogen, methyl, ethyl, halogen (in particular fluor (F)), CF₃ and isopropyl.

Such a 6-membered ring can also be schematically represented by the following structure (Scheme D), in which the optional substituents are indicated as R₅ to R₁₂:

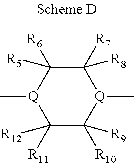

in which:
each Q is independently a carbon atom or a nitrogen atom, preferably such that at least one Q is a nitrogen atom, and more preferably such that both atoms Q are nitrogen atoms;
   and:
each of R₅, R₆, R₇, R₈, R₉, R₁₀, R₁₁ and R₁₂ are preferably independently chosen from the group consisting of hydrogen, methyl, ethyl, halogen (in particular fluor (F)), CF₃ and isopropyl.

By means of some preferred, but non-limiting illustrative examples, the Aliphatic Ring A may for example be:
an (optionally suitably substituted) cyclohexane ring, to which the Aromatic Ring System C is linked (i.e. either directly or via an alkylene linking group and in particular a methylene linking group, both as defined herein) at the “1” position of the cyclohexane ring and the Aromatic Ring B is linked (i.e. either directly or via an alkylene linking group and in particular a methylene linking group, both as defined herein) at the “4” position of the cyclohexane ring;
   and may in particular be:
a (optionally suitably substituted) piperidine ring, in which the Aromatic Ring System C is linked (i.e. either directly or via an alkylene linking group as defined herein) to the nitrogen atom of the piperidine ring and the Aromatic Ring B is linked (i.e. either directly or via an alkylene linking group and in particular a methylene linking group, both as defined herein) at the “4” position of the piperidine ring (or vice versa, with the Aromatic Ring B linked to the nitrogen atom and the Aromatic Ring System C at the “4” position); and may more in particular be:
an (optionally suitably substituted) piperazine ring, to which the Aromatic Ring System C is linked (i.e. either directly or via an alkylene linking group and in particular a methylene linking group, both as defined herein) to the nitrogen atom at the “1” position and to which the Aromatic Ring B is linked (i.e. either directly or via an alkylene linking group and in particular a methylene linking group, both as defined herein) to the nitrogen atom at the “4” position of the piperazine ring.

As described herein, as an alternative to a structure of Scheme C or Scheme D, the Aliphatic Ring A may be a ring system of one of Formulae XCIX or C:

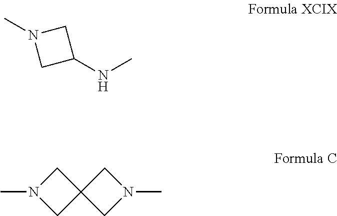

in which said ring system of Formula XCIX or Formula C may optionally be suitably substituted with one or more suitable substituents that, when present, are each preferably independently chosen from the group consisting of hydrogen, methyl, ethyl, halogen (in particular fluor (F)), CF₃ and isopropyl.

Some non-limiting examples of aliphatic rings that can be present in the compounds of the invention as the Aliphatic Ring A include the following:

TABLE E

| Formula | Structure |
|---|---|
| LXXXIV | 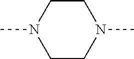 |
| LXXXV | 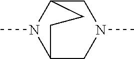 |
| LXXXVI | 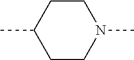 |
| LXXXVII | 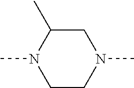 |
| LXXXVIII | 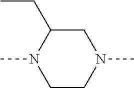 |
| LXXXIX | 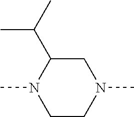 |
| XC | 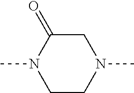 |
| XCI | 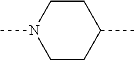 |
| XCII | 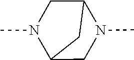 |
| XCIII | 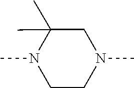 |
| XCIV | 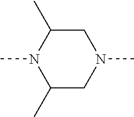 |
| XCV | 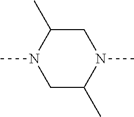 |
| XCVI | 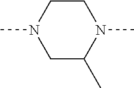 |
| XCVII | 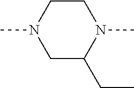 |
| XCVIII | 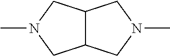 |
| XCIX | 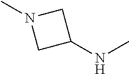 |
| C | 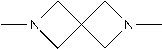 |
| — | — |

As will be clear to the skilled person based on the disclosure herein as well as the compounds of the invention that are exemplified in the Experimental Part below, the Aliphatic Ring A (in its various aspects as disclosed herein) can be unsubstituted or substituted. As will also be clear to the skilled person when the Aliphatic Ring A is unsubstituted, each R_{X} and R_{y} in Scheme C, and each of R₅ to R₁₂ in Scheme D will be a hydrogen atom.

As also mentioned herein, when the Aliphatic Ring A is substituted, each substituent that is present can generally be independently chosen from the group consisting of methyl, ethyl, fluor (F), CF₃ and isopropyl (and are preferably independently chosen from the group consisting of methyl, ethyl and isopropyl). Thus, generally, and as will be clear to the skilled person, when the Aliphatic Ring A is substituted, each Rₓ and R_{y} in Scheme C, and each of R₅ to R₁₂ in Scheme D, can be independently chosen from the group consisting of hydrogen, methyl, ethyl, fluor (F), CF₃ and isopropyl (and are preferably independently chosen from the group consisting of methyl, ethyl and isopropyl), provided that: (i) with respect to Scheme C, at least one of the groups Rₓ and R_{y} is chosen from the group consisting of methyl, ethyl, fluor (F), CF₃ and isopropyl (and is preferably chosen from the group consisting of methyl, ethyl and isopropyl); and/or that: (ii) with respect to Scheme D, at least one of the groups R₅ to R₁₂ is chosen from the group consisting of methyl, ethyl, fluor (F), CF₃ and isopropyl (and is preferably chosen from the group consisting of methyl, ethyl and isopropyl).

When the Aliphatic Ring A is substituted with two or more substituents as described herein, said substituents can suitably be the same or different. Also, each of said substituents can suitably be present on different carbon atoms in the Aliphatic Ring A, but it is also possible that one carbon atom in the Aliphatic Ring A suitably carries two such substituents (provided that such carbon atom can suitably carry two substituents). It will also be clear to the skilled person that, where a carbon atom in the Aliphatic Ring A carries a single substituent, that such carbon atom may in addition also suitably carry a hydrogen atom.

It will also be clear to the skilled person that the maximum number of substituents that can be present on the Aliphatic Ring A will depend on the number of carbon atom that are present in the Aliphatic Ring A and on the number of substituents that each such carbon atom can carry. Thus, by means of example and without limitation, in the 6-membered Aliphatic Ring A that is schematically represented by Scheme D, the maximum number of substituents that can be present on Ring A is 8 (i.e. when each of R₅ to R₁₂ is a substituent as described herein).

Generally however, and without limitation, the total number of substituents on Ring A will be 0, 1, 2, 3, 4, 5 or 6, preferably 0, 1, 2, 3 or 4, and more preferably 0, 1 or 2. Again, as described herein, such substituents can be the same or different, and can be present on different carbon atoms in the Aliphatic Ring A, but it is also possible that one carbon atom in the Aliphatic Ring A suitably carries two such substituents (provided that such carbon atom can suitably carry two substituents).

According to one specific, but non-limiting aspect, the total number of substituents on Ring A will be 0, 1, 2, 3 or 4 (and is preferably 0, 1 or 2), in which any substituents that are present on the Aliphatic Ring A are preferably independently chosen from methyl, ethyl and isopropyl (and are preferably methyl). Again, as described herein, such substituents can be the same or different, and can be present on different carbon atoms in the Aliphatic Ring A, but it is also possible that one carbon atom in the Aliphatic Ring A suitably carries two such substituents (provided that such carbon atom can suitably carry two substituents).

Some preferred but non-limiting examples of compounds of the invention in which the Aliphatic Ring A is substituted are given in the Experimental Part below as Compounds A-173 to A-188, Compounds A-216 to A-218, Compounds A-222 to A-224 and Compounds A-226 to A-229. It should be noted that the Aliphatic Ring A in other compounds of the invention can be substituted in the same way (i.e. carry the same substituent(s) on the same carbon atom(s)) as the Aliphatic Ring A is substituted in one of said Compounds A-173 to A-188, Compounds A-216 to A-218, Compounds A-222 to A-224 and Compounds A-226 to A-229 (in other words, other compounds of the invention can contain a substituted Aliphatic Ring A that is the same as the substituted Aliphatic Ring A that is present in one of the Compounds as Compounds A-173 to A-188, Compounds A-216 to A-218, Compounds A-222 to A-224 and Compounds A-226 to A-229).

Also, when the Aliphatic Ring A is substituted with one or more substituents as described herein, said substituents can be suitably present on any carbon atom in the Aliphatic Ring A that can suitably carry one or more such substituents. According to one specific but non-limiting aspect, when the Aliphatic Ring A is substituted with one or more substituents as described herein, at least one of these substituents is present on a carbon atom that, in Ring A, is adjacent to the carbon or nitrogen atom (and preferably nitrogen atom) in the Aliphatic Ring A to which the Aromatic Ring System C is linked. According to an even more specific, but non-limiting aspect, where the Aliphatic Ring A carries 1, 2, 3 or 4 such substituents (as described herein), and in particular 1 or 2 such substituents, all said substituents are suitably present on one or both of the carbon atoms that in Ring A, are adjacent to the carbon or nitrogen atom (and preferably nitrogen atom) in the Aliphatic Ring A to which the Aromatic Ring System C is linked. Again, such substituents are preferably independently chosen from methyl, ethyl and isopropyl, and are most preferably methyl.

In an even more particular aspect, in a compound of the invention, the Aliphatic Ring A is unsubstituted or is substituted with 1 or 2 substituents (and preferably one substituent) chosen from methyl, ethyl and isopropyl (and that preferably is/are methyl), in which said substituent(s) are present on the carbon atom(s) in the Aliphatic Ring A that are adjacent to the carbon or nitrogen atom (and preferably nitrogen atom) in the Aliphatic Ring A to which the Aromatic Ring System C is linked.

Further aspects of the invention relate to compounds of the invention (and in particular compounds of Formula I, II, III, IV, V or VI) in which the Aliphatic Ring A is unsubstituted or substituted as described herein (and in particular as described in the preceding paragraphs), in which such a compound of the invention is preferably further such that such compound has an affinity for AT2R (measured according to the protocol set out in Example 2 below) that is better than 10 micromolar, preferably better than 1 micromolar, more preferably better than 0.1 micromolar, even more preferably better than 10 nanomolar (and for the remainder is as further described herein).

Some specific but non-limiting examples of compounds of the invention (including such compounds of the invention that are particularly preferred) that contain an Aliphatic Ring A as described herein will become clear to the skilled person based on the disclosure herein and/or are as exemplified in the Experimental Part below.

As described herein, the Aromatic Ring B will generally be a 6-membered aromatic ring that comprises carbon atoms and may optionally (and suitably) contain 1, 2 or 3 nitrogen atoms, provided that the Aromatic Ring B is at least still such that it can carry the Acidic Substituent D in a position ortho relative to the Aliphatic Ring A (i.e. as further described herein). Also, when the Aromatic Ring B contains one or more nitrogen-atoms, it is preferably further such that it can still carry the one or more substituents or combination of such substituents that are described herein as being preferably present on the Aromatic Ring B (i.e. in the positions on the Aromatic Ring B that are described herein as being preferred for said substituents); and more preferably such that it can still carry the one or more substituents or combination of such substituents that are described herein as being particularly or most preferred, respectively, for being on the Aromatic Ring B (i.e. in the positions on the Aromatic Ring B that are described herein as being particularly or most preferred, respectively, for said substituents).

Some preferred examples of aromatic rings that can form the 6-membered ring within the Aromatic Ring B are phenyl (benzene), pyridin-2-yl and 1,4-pyrimidin-2-yl, with phenyl being most preferred.

As described herein, most preferably the Aromatic Ring B will be a 6-membered ring, although in the invention in its broadest sense, it is not excluded that the Aromatic Ring B may be a 5-membered aromatic ring (which may optionally also suitably contain 1 or 2 hetero-atoms chosen from O, N or S), provided that such a 5-membered aromatic ring is such that it can still carry the one or more substituents or combination of such substituents that are described herein as being preferably present on a 6-membered Aromatic Ring B. More preferably, when such a 5-membered aromatic ring is present instead of a 6-membered aromatic ring, the resulting 5-membered Aromatic Ring B is preferably such that it is a bio-isostere of the 6-membered Aromatic Rings B described herein, and more preferably a bio-isostere of the 6-membered Aromatic Ring B represented by Scheme E, even more preferably a bioisostere of the 6-membered Aromatic Ring B represented by Scheme F, and most preferably a bioisostere of the 6-membered Aromatic Ring B represented by Scheme G.

The Aromatic Ring B will most preferably be a monocyclic ring, as the use of a bicyclic aromatic ring system as the Aromatic Ring B will often result in a compound that cannot suitably comprise the preferred substituents that are described herein for the Aromatic Ring B (such as an isobutyl group in position 5 and a fluorine in one of positions 3 or 4).

As further described herein, the Aromatic Ring B will carry an acidic substituent (i.e. the Acidic Substituent D) on a carbon atom of Ring B that is adjacent to the carbon atom at which the Aliphatic Ring A is linked to the Aromatic Ring B. Said Acidic Substituent D can be any suitable acidic group or substituent (with suitable groups/substituents being clear to the skilled person based on the disclosure herein, optionally after a limited degree of trial-and-error) and is preferably chosen from the group consisting of a carboxylic acid group (i.e. a —C(═O)—OH group), acylsulfonamide groups (such as, for example and without limitation, an acylsulfonamide group of the general formula CONHSO₂R or SO₂NHCOR wherein R is —C₁-C₈ alkyl, CH₂(C₁-C₈ cycloalkyl), CH₂(heterocyclyl with 1-6 ring atoms), C₁-C₈ alkoxy or C₁-C₈ amine), a tetrazole group or a group that is a bioisostere (as defined herein) of a tetrazole group (such as, for example and without limitation, a 4H-1,2,4-oxadiazol-5-one group) and is more preferably tetrazole or a bioisostere thereof, and most preferably tetrazole.

As also further described herein, the Aromatic Ring B will preferably carry at least one further substituent that is chosen from the group consisting of: methyl, ethyl, propyl, isopropyl, cyclopropyl, methylcyclopropyl, n-butyl, sec-butyl, isobutyl, cyclobutyl, methylcyclobutyl, vinyl, allyl, isobutenyl, trifluoromethyl, methoxy, ethoxy, n-propoxy, isopropoxy, isobutoxy, difluoroethoxy, methoxyethyloxy, fluorine, chlorine, cyano, oxirane, cyclopropoxy, cyclobutoxy, cyclopentoxy, N-acetyl, substituted and unsubstituted carbamoyl, which substituent (when present) is most preferably present on a carbon atom in the Ring B that is in a position para to the position In Ring B of the carbon atom to which the Acidic Substituent D is linked (e.g. position “5” according to the numbering system used wherein when the Aromatic Ring B is a 6-membered aromatic ring such as a phenyl ring), in which case said substituent will form the substituent R₃ as indicated in Schemes E and F below and some of the further Schemes and Formulae provided herein. Said substituent is preferably an isobutyl group, as for example illustrated by the compounds of Formulae III and VI.

As also further described herein, it is also possible that, in addition to the Acidic Substituent [D] and said at least one further substituent chosen from the group consisting of: methyl, ethyl, propyl, isopropyl, n-butyl, sec-butyl, isobutyl, methoxy, ethoxy, n-propoxy, isopropoxy, methylmethoxy (which substituent, as mentioned herein, is preferably isobutyl and is in the para position relative to Acidic Substituent D), the Aromatic Ring B may optionally be further suitably substituted with one or more (such as 1 or 2) suitable substituents (as defined herein), which substituents are indicated as R₁, R₂ and R₄ in Schemes E to G and Formulae I to VI herein.

Thus, according to a specific but non-limiting aspect, the Aromatic Ring B has an overall structure that can be schematically represented as follows (Scheme E):

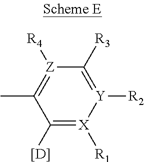

in which:
the acidic substituent denoted by [D] is chosen from the group consisting of a carboxylic acid (—COOH) group, an acylsulfonamide group (such as an acylsulfonamide group of the general formula CONHSO₂R or SO₂NHCOR wherein R is —C₁-C₈ alkyl, CH₂(C₁-C₈ cycloalkyl), CH₂(heterocyclyl with 1-6 ring atoms), C₁-C₈ alkoxy or C₁-C₈ amine), a tetrazole group or a group that is a bioisostere (as defined herein) of a tetrazole group (such as, for example and without limitation, a 4H-1,2,4-oxadiazol-5-one group), and is more preferably tetrazole or a bioisostere thereof, and most preferably tetrazole;
   and:
X and Y are each either a nitrogen atom or a carbon atom, such that either both X and Y are carbon atoms or only one or X or Y is a nitrogen atom (with the other of X and Y being a carbon atom);
   and:
Z is a nitrogen atom or a carbon atom;
   and:
R₁, when present, is chosen from the group consisting of H, halogen (F, Cl, Br or I, and preferably F or Cl), C₁-C₈ alkyl, C₃ or C₄ cycloalkyl, CF₃, C₁-C₈ alkoxy, amine (—NH₂) or C₁-C₂ substituted amine (e.g. dimethylamine or diethylamine) and cyano;
   and:
R₂, when present, is chosen from the group consisting of H, halogen (F, Cl, Br or I, and preferably F or Cl), C₁-C₈ alkyl, C₁-C₈ alkoxy, cycloalkyl, amine (—NH₂) or C₁-C₂ substituted amine (e.g. dimethylamine or diethylamine);
   and:
R₃ is chosen from the group consisting of H, halogen (F, Cl, Br or I, and preferably F or Cl), C₁₋₈ alkyl (e.g. sec-butyl and in particular iso-butyl), including fluor-substituted methyl groups (e.g. trifluoromethyl and CHF₂), C₁-C₈ alkoxy, —O—CF₃, methoxyethyloxy (—O—(CH₂)₂O—CH₃) or difluoroethoxy (—O—CH₂—CHF₂), cycloalkyl (e.g. cyclopropoxy, cyclobutoxy or cyclopentoxy), —CH₂-cycloalkyl, —O—CH₂-cycloalkyl, —O-cycloalkyl, —NH— cycloalkyl, —N(C₁-C₃)-cycloalkyl, —NH-heteroalkyl, —N(C₁-C₃)-heteroalkyl, heterocyclyl (e.g. oxirane), —CH₂-heterocyclyl, —O—CH₂-heterocyclyl (e.g. —O—CH₂-oxirane), —O— heterocyclyl, —NH-heterocyclyl, —N(C₁-C₃)-heterocyclyl, vinyl or methyl-substituted vinyl (e.g. —CH═CHCH₃, —CH═C(CH₃)₂ or —CH═CH₂), or allyl or methyl-substituted allyl (e.g. —CH₂CH═CH₂), isobutenyl or methyl substituted isobutenyl (e.g. ═C(CH₃)₂) and cyano; and is most preferably isobutyl (i.e. as in the compounds of the invention of Formula III and/or Formula VI); and:
R₄, when present, is chosen from the group consisting of H or halogen (F, Cl, Br or I, and preferably F or Cl);
   and provided that:
X, Y and Z are chosen such (i.e. each from a carbon atom or a nitrogen atom) that the resulting ring structure form an aromatic ring (i.e. a planar conjugated ring);
   and further provided that:
when X is a nitrogen atom, R₁ is not present;
   and further provided that:
when Y is a nitrogen atom, R₂ is not present;
   and further provided that:
when Z is a nitrogen atom, R₄ is not present.

Preferably, only one of X, Y and Z is a nitrogen atom (with the others of X, Y and Z being carbon atoms), and more preferably only one of Y and Z is a nitrogen atom (with X being a carbon atom and the other of Y and Z also being a carbon atom, and even more preferably Z is a nitrogen atom (with both X and Y being carbon atoms). Most preferably, all of X, Y and Z are carbon atoms (i.e. such that Ring B is a substituted phenyl ring without any nitrogen atoms).

According to a preferred but non-limiting aspect, the Aromatic Ring B has an overall structure that can be schematically represented as follows (Scheme F):

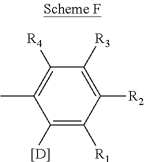

and thus comprises or essentially consists of a phenyl ring that is substituted with the acidic substituent denoted by [D] and the substituents R₁, R₂, R₃ and/or R₄, in which:
the acidic substituent denoted by [D] is chosen from the group consisting of a carboxylic acid (—COOH) group, an acylsulfonamide group (such as an acylsulfonamide group of the general formula CONHSO₂R or SO₂NHCOR wherein R is —C₁-C₈ alkyl, CH₂(C₁-C₈ cycloalkyl), CH₂(heterocyclyl with 1-6 ring atoms), C₁-C₈ alkoxy or C₁-C₈ amine), a tetrazole group or a group that is a bioisostere (as defined herein) of a tetrazole group (such as, for example and without limitation, a 4H-1,2,4-oxadiazol-5-one group), and is more preferably tetrazole or a bioisostere thereof, and most preferably tetrazole;
   and:
R₁ is chosen from the group consisting of H, halogen (F, Cl, Br or I, and preferably F or Cl), C₁-C₈ alkyl, C₃ or C₄ cycloalkyl, CF₃, C₁-C₈ alkoxy, amine (—NH₂) or C₁-C₂ substituted amine (e.g. dimethylamine or diethylamine) and cyano;
   and:
R₂ is chosen from the group consisting of H, halogen (F, Cl, Br or I, and preferably F or Cl), C₁-C₈ alkyl, C₁-C₈ alkoxy, cycloalkyl, amine (—NH₂) or C₁-C₂ substituted amine (e.g. dimethylamine or diethylamine);
   and:
R₃ is chosen from the group consisting of H, halogen (F, Cl, Br or I, and preferably F or Cl), C₁₋₈ alkyl (e.g. sec-butyl and in particular iso-butyl), including fluor-substituted methyl groups (e.g. trifluoromethyl and CHF₂), C₁-C₈ alkoxy, —O—CF₃, methoxyethyloxy (—O—(CH₂)₂O—CH₃) or difluoroethoxy (—O—CH₂—CHF₂), cycloalkyl (e.g. cyclopropoxy, cyclobutoxy or cyclopentoxy), —CH₂-cycloalkyl, —O—CH₂-cycloalkyl, —O-cycloalkyl, —NH— cycloalkyl, —N(C₁-C₃)-cycloalkyl, —NH-heteroalkyl, —N(C₁-C₃)-heteroalkyl, heterocyclyl (e.g. oxirane), —CH₂-heterocyclyl, —O—CH₂-heterocyclyl (e.g. —O—CH₂-oxirane), —O— heterocyclyl, —NH-heterocyclyl, —N(C₁-C₃)-heterocyclyl, vinyl or methyl-substituted vinyl (e.g. —CH═CHCH₃, —CH═C(CH₃)₂ or —CH═CH₂), or allyl or methyl-substituted allyl (e.g. —CH₂CH═CH₂), isobutenyl or methyl substituted isobutenyl (e.g. ═C(CH₃)₂) and cyano; and is most preferably isobutyl (i.e. as in the compounds of the invention of Formula III and/or Formula VI); and:
R₄ is chosen from the group consisting of H or halogen (F, Cl, Br or I, and preferably F or Cl).

According to a particularly preferred but non-limiting aspect, the Aromatic Ring B has an overall structure that can be schematically represented as follows (Scheme G):

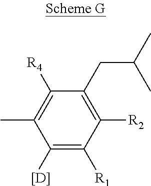

in which:
the acidic substituent denoted by [D] is chosen from the group consisting of a carboxylic acid (—COOH) group, an acylsulfonamide group (such as an acylsulfonamide group of the general formula CONHSO₂R or SO₂NHCOR wherein R is —C₁-C₈ alkyl, CH₂(C₁-C₈ cycloalkyl), CH₂(heterocyclyl with 1-6 ring atoms), C₁-C₈ alkoxy or C₁-C₈ amine), a tetrazole group or a group that is a bioisostere (as defined herein) of a tetrazole group (such as, for example and without limitation, a 4H-1,2,4-oxadiazol-5-one group), and is more preferably tetrazole or a bioisostere thereof, and most preferably tetrazole;
   and:
R₁ is chosen from the group consisting of H, halogen (F, Cl, Br or I, and preferably F or Cl), C₁-C₈ alkyl, C₃ or C₄ cycloalkyl, CF₃, C₁-C₈ alkoxy, amine (—NH₂) or C₁-C₂ substituted amine (e.g. dimethylamine or diethylamine) and cyano;
and:
R₂ is chosen from the group consisting of H, halogen (F, Cl, Br or I, and preferably F or Cl), C₁-C₈ alkyl, C₁-C₈ alkoxy, cycloalkyl, amine (—NH₂) or C₁-C₂ substituted amine (e.g. dimethylamine or diethylamine);
   and:
R₄ is chosen from the group consisting of H or halogen (F, Cl, Br or I, and preferably F or Cl).

Some specific but non-limiting examples of compounds of the invention (including such compounds of the invention that are particularly preferred) that contain an Aromatic Ring B as described herein will become clear to the skilled person based on the disclosure herein and/or are as exemplified in the Experimental Part below.

In a specific, but non-limiting aspect, the invention relates to a compound of Formula I:

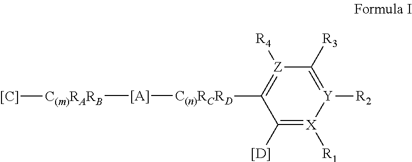

in which:
[C] is a monocyclic or polycyclic (and preferably monocyclic or bicyclic) aromatic ring system, such that: (i) the aromatic ring system contains at least one aromatic ring; (ii) the aromatic ring system is linked to the remainder of the compound of Formula I (i.e. either directly via a covalent bond or via the alkylene linking group —C₍ₘ₎R_{A}R_{B}— when said alkylene linking group is present) via a carbon atom that is present in said at least one aromatic ring; and (iii) said at least one aromatic ring (i.e. the aromatic ring that contains the carbon atom to which the remainder of the compound of Formula I is linked) preferably contains at least one (such as 1 or 2) hetero-atom(s), which hetero-atoms (when present) are preferably each independently and suitably chosen from N, S and O and are more preferably such that at least one of the hetero-atoms (when present) is a nitrogen atom; with the aromatic ring system denoted as [C] preferably being an aromatic ring or ring system that is chosen from the group consisting of the aromatic rings/ring systems of Formulae VII to LXXXIII, more preferably from the group consisting of the aromatic rings/ring systems of Formulae LIX to LXXXIII, and even more preferably from the group consisting of the aromatic rings/ring systems of Formulae LXXIII to LXXXIII;
   and:
m is 1 or 0 and n is 1 or 0, such that the sum of m and n is either 2, 1 or 0 and is preferably 1 or 0 (it being understood that when m=0, R_{A} and R_{B} will not be present and that when n=0, R_{C} and R_{D} will not be present), with m preferably being 1 and n preferably being 0;
   and:
each of R_{A}, R_{B}, R_{C} and R_{D}, when present, is independently chosen from hydrogen, methyl and/or trifluoromethyl, or where R_{A}+R_{B}, when present, together with the carbon atom to which they are bound form a carbonyl (C═O) group; or R_{C}+R_{D}, when present, together with the carbon atom to which they are bound form a carbonyl (C═O) group (in other words, in which R_{A}+R_{B} together are replaced by a single oxygen atom so as to form a carbonyl group with the carbon atom to which said oxygen atom is bound, or in which R_{C}+R_{D}, when present, together are replaced by a single oxygen atom so as to form a carbonyl group with the carbon atom to which said oxygen atom is bound), and are preferably each a hydrogen atom;
   and:
[A] is either a ring system that is as schematically represented by the following Scheme C:

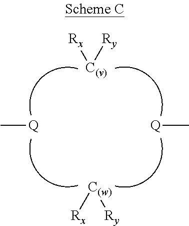

in which each atom Q is independently a carbon atom or a nitrogen atom (and preferably such that at least one atom Q is a nitrogen atom, and more preferably such that both atoms Q are nitrogen atoms), v is an integer between 1 and 4 (i.e. 1, 2, 3 or 4) and w is an integer between 1 and 4 (i.e. 1, 2, 3 or 4), such that the sum of (v+w) is 3, 4, 5, 6, 7 or 8 (and preferably 3, 4 or 5, with v and w more preferably both being 2) and such that the difference (v-w) is either 1, 0 or −1, and each Rₓ that is present and each R_{y} that is present is preferably independently chosen from the group consisting of hydrogen, methyl, ethyl, fluor (F), CF₃ and isopropyl; and in particular a ring system that is as schematically represented by the following Scheme D:

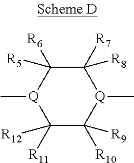

in which each atom Q is independently a carbon atom or a nitrogen atom (and preferably such that at least one atom Q is a nitrogen atom, and more preferably such that both atoms Q are nitrogen atoms) and each of R₅, R₆, R₇, R₈, R₉, R₁₀, R₁₁ and R₁₂ are preferably independently chosen from the group consisting of hydrogen, methyl, ethyl, fluor (F), CF₃ and isopropyl; or alternatively a ring system of formula XCIX or C

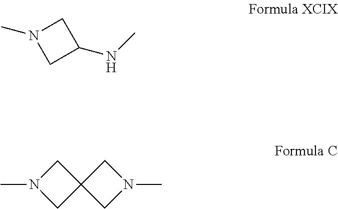

in which said ring system of Formula XCIX or Formula C may optionally be suitably substituted with one or more suitable substituents that, when present, are each preferably independently chosen from the group consisting of hydrogen, methyl, ethyl, fluor (F), CF₃ and isopropyl;
   and:
the acidic substituent denoted by [D] is chosen from the group consisting of a carboxylic acid (—COOH) group, an acylsulfonamide group (such as an acylsulfonamide group of the general formula CONHSO₂R or SO₂NHCOR wherein R is —C₁-C₈ alkyl, CH₂(C₁-C₈ cycloalkyl), CH₂(heterocyclyl with 1-6 ring atoms), C₁-C₈ alkoxy or C₁-C₈ amine), a tetrazole group or a group that is a bioisostere (as defined herein) of a tetrazole group (such as, for example and without limitation, a 4H-1,2,4-oxadiazol-5-one group), and is more preferably tetrazole or a bioisostere thereof, and most preferably tetrazole;
   and:
X and Y are each either a nitrogen atom or a carbon atom, such that either both X and Y are carbon atoms or only one or X or Y is a nitrogen atom (with the other of X and Y being a carbon atom);
   and:
Z is a nitrogen atom or a carbon atom;
   and:
R₁, when present, is chosen from the group consisting of H, halogen (F, Cl, Br or I, and preferably F or Cl), C₁-C₈ alkyl, C₃ or C₄ cycloalkyl, CF₃, C₁-C₈ alkoxy, amine (—NH₂) or C₁-C₂ substituted amine (e.g. dimethylamine or diethylamine) and cyano;
   and:
R₂, when present, is chosen from the group consisting of H, halogen (F, Cl, Br or I, and preferably F or Cl), C₁-C₈ alkyl, C₁-C₈ alkoxy, cycloalkyl, amine (—NH₂) or C₁-C₂ substituted amine (e.g. dimethylamine or diethylamine);
   and:
R₃ is chosen from the group consisting of H, halogen (F, Cl, Br or I, and preferably F or Cl), C₁₋₈ alkyl (e.g. sec-butyl and in particular iso-butyl), including fluor-substituted methyl groups (e.g. trifluoromethyl and CHF₂), C₁-C₈ alkoxy, —O—CF₃, methoxyethyloxy (—O—(CH₂)₂O—CH₃) or difluoroethoxy (—O—CH₂—CHF₂), cycloalkyl (e.g. cyclopropoxy, cyclobutoxy or cyclopentoxy), —CH₂-cycloalkyl, —O—CH₂-cycloalkyl, —O-cycloalkyl, —NH— cycloalkyl, —N(C₁-C₃)-cycloalkyl, —NH-heteroalkyl, —N(C₁-C₃)-heteroalkyl, heterocyclyl (e.g. oxirane), —CH₂-heterocyclyl, —O—CH₂-heterocyclyl (e.g. —O—CH₂-oxirane), —O— heterocyclyl, —NH-heterocyclyl, —N(C₁-C₃)-heterocyclyl, vinyl or methyl-substituted vinyl (e.g. —CH═CHCH₃, —CH═C(CH₃)₂ or —CH═CH₂), or allyl or methyl-substituted allyl (e.g. —CH₂CH═CH₂), isobutenyl or methyl substituted isobutenyl (e.g. ═C(CH₃)₂) and cyano; and is most preferably isobutyl;
   and:
R₄, when present, is chosen from the group consisting of H or halogen (F, Cl, Br or I, and preferably F or Cl);
   and provided that:
X, Y and Z are chosen such (i.e. each from a carbon atom or a nitrogen atom) that the resulting ring structure form an aromatic ring (i.e. a planar conjugated ring);
   and further provided that:
when X is a nitrogen atom, R₁ is not present;
   and further provided that:
when Y is a nitrogen atom, R₂ is not present;
   and further provided that:
when Z is a nitrogen atom, R₄ is not present.

As further described herein, the invention in particular aspect relates to such a compound of Formula I that has an affinity for AT2R (measured according to the protocol set out in Example 2 below) that is better than 10 micromolar, preferably better than 1 micromolar, more preferably better than 0.1 micromolar, even more preferably better than 10 nanomolar. In a more particular aspect, the invention relates to a compound of Formula I in which: (i) the Aromatic Ring System [C], the Aliphatic Ring [A] and the acidic substituent [D] are each as further described herein; and (ii) each of X, Y and Z, as well as the particular combination of the atoms X, Y and Z that is present in said compound of Formula I; and (iii) each of the substituents R₁, R₂, R₃ and R₄ (when present) as well as the particular combination of such substituents R₁, R₂, R₃ and R₄ that is present in said compound of Formula I; and (iv) m and n (which as described herein can each independently be 0 or 1); and (v) each of the substituents R_{A}, R_{B}, R_{C} and R_{D} (when present) as well as the particular combination of such substituents R_{A}, R_{B}, R_{C} and R_{D} that is present in said compound of Formula I; and (vi) any further substituents that are present in such a compound of Formula I (e.g. on the aromatic ring system [C] and/or on the aliphatic ring system [A], as further described herein) as well as the particular combination of such substituents that is present in said compound of Formula I, are each such that (and are in combination such that) said compound of Formula I has an affinity for AT2R (measured according to the protocol set out in Example 2 below) that is better than 10 micromolar, preferably better than 1 micromolar, more preferably better than 0.1 micromolar, even more preferably better than 10 nanomolar.

In a further specific, but non-limiting aspect, the invention relates to a compound of Formula II:

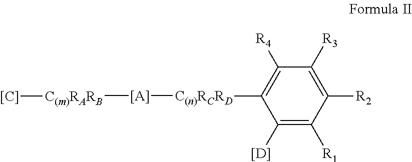

in which:
[C] is a monocyclic or polycyclic (and preferably monocyclic or bicyclic) aromatic ring system, such that: (i) the aromatic ring system contains at least one aromatic ring; (ii) the aromatic ring system is linked to the remainder of the compound of Formula II (i.e. either directly via a covalent bond or via the alkylene linking group —C₍ₘ₎R_{A}R_{B}— when said alkylene linking group is present) via a carbon atom that is present in said at least one aromatic ring; and (iii) said at least one aromatic ring (i.e. the aromatic ring that contains the carbon atom to which the remainder of the compound of Formula II is linked) preferably contains at least one (such as 1 or 2) hetero-atom(s), which hetero-atoms (when present) are preferably each independently and suitably chosen from N, S and O and are more preferably such that at least one of the hetero-atoms (when present) is a nitrogen atom; with the aromatic ring system denoted as [C] preferably being an aromatic ring or ring system that is chosen from the group consisting of the aromatic rings/ring systems of Formulae VII to LXXXIII, more preferably from the group consisting of the aromatic rings/ring systems of Formulae LIX to LXXXIII, and even more preferably from the group consisting of the aromatic rings/ring systems of Formulae LXXIII to LXXXIII;
   and:
m is 1 or 0 and n is 1 or 0, such that the sum of m and n is either 2, 1 or 0 and is preferably 1 or 0 (it being understood that when m=0, R_{A} and R_{B} will not be present and that when n=0, R_{C} and R_{D} will not be present), with m preferably being 1 and n preferably being 0;
   and:
each of R_{A}, R_{B}, R_{C} and R_{D}, when present, is independently chosen from hydrogen, methyl and/or trifluoromethyl, or where R_{A}+R_{B}, when present, together with the carbon atom to which they are bound form a carbonyl (C═O) group; or R_{C}+R_{D}, when present, together with the carbon atom to which they are bound form a carbonyl (C═O) group (in other words, in which R_{A}+R_{B} together are replaced by a single oxygen atom so as to form a carbonyl group with the carbon atom to which said oxygen atom is bound, or in which R_{C}+R_{D}, when present, together are replaced by a single oxygen atom so as to form a carbonyl group with the carbon atom to which said oxygen atom is bound), and are preferably each a hydrogen atom;
   and:
[A] is either a ring system that is as schematically represented by the following Scheme C:

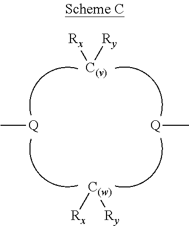

in which each atom Q is independently a carbon atom or a nitrogen atom (and preferably such that at least one atom Q is a nitrogen atom, and more preferably such that both atoms Q are nitrogen atoms), v is an integer between 1 and 4 (i.e. 1, 2, 3 or 4) and w is an integer between 1 and 4 (i.e. 1, 2, 3 or 4), such that the sum of (v+w) is 3, 4, 5, 6, 7 or 8 (and preferably 3, 4 or 5, with v and w more preferably both being 2) and such that the difference (v-w) is either 1, 0 or −1, and each Rₓ that is present and each R_{y} that is present is preferably independently chosen from the group consisting of hydrogen, methyl, ethyl, fluor (F), CF₃ and isopropyl; and in particular a ring system that is as schematically represented by the following Scheme D:

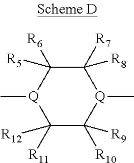

in which each atom Q is independently a carbon atom or a nitrogen atom (and preferably such that at least one atom Q is a nitrogen atom, and more preferably such that both atoms Q are nitrogen atoms) and each of R₅, R₆, R₇, R₈, R₉, R₁₀, R₁₁ and R₁₂ can independently be a suitable substituent that is preferably independently chosen from the group consisting of hydrogen, methyl, ethyl, fluor (F), CF₃ and isopropyl; or alternatively a ring system of formula XCIX or C

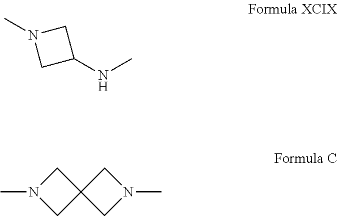

in which said ring system of Formula XCIX or Formula C may optionally be suitably substituted with one or more suitable substituents that, when present, are each preferably independently chosen from the group consisting of hydrogen, methyl, ethyl, fluor (F), CF₃ and isopropyl;
   and:
the acidic substituent denoted by [D] is chosen from the group consisting of a carboxylic acid (—COOH) group, (such as an acylsulfonamide group of the general formula CONHSO₂R or SO₂NHCOR wherein R is —C₁-C₈ alkyl, CH₂(C₁-C₈ cycloalkyl), CH₂(heterocyclyl with 1-6 ring atoms), C₁-C₈ alkoxy or C₁-C₈ amine), a tetrazole group or a group that is a bioisostere (as defined herein) of a tetrazole group (such as, for example and without limitation, a 4H-1,2,4-oxadiazol-5-one group), and is more preferably tetrazole or a bioisostere thereof, and most preferably tetrazole;
   and:
R₁ is chosen from the group consisting of H, halogen (F, Cl, Br or I, and preferably F or Cl), C₁-C₈ alkyl, C₃ or C₄ cycloalkyl, CF₃, C₁-C₈ alkoxy, amine (—NH₂) or C₁-C₂ substituted amine (e.g. dimethylamine or diethylamine) and cyano;
   and:
R₂ is chosen from the group consisting of H, halogen (F, Cl, Br or I, and preferably F or Cl), C₁-C₈ alkyl, C₁-C₈ alkoxy, cycloalkyl, amine (—NH₂) or C₁-C₂ substituted amine (e.g. dimethylamine or diethylamine);
   and:
R₃ is chosen from the group consisting of H, halogen (F, Cl, Br or I, and preferably F or Cl), C₁₋₈ alkyl (e.g. sec-butyl and in particular iso-butyl), including fluor-substituted methyl groups (e.g. trifluoromethyl and CHF₂), C₁-C₈ alkoxy, —O—CF₃, methoxyethyloxy (—O—(CH₂)₂O—CH₃) or difluoroethoxy (—O—CH₂—CIF₂), cycloalkyl (e.g. cyclopropoxy, cyclobutoxy or cyclopentoxy), —CH₂-cycloalkyl, —O—CH₂-cycloalkyl, —O-cycloalkyl, —NH— cycloalkyl, —N(C₁-C₃)-cycloalkyl, —NH-heteroalkyl, —N(C₁-C₃)-heteroalkyl, heterocyclyl (e.g. oxirane), —CH₂-heterocyclyl, —O—CH₂-heterocyclyl (e.g. —O—CH₂-oxirane), —O— heterocyclyl, —NH-heterocyclyl, —N(C₁-C₃)-heterocyclyl, vinyl or methyl-substituted vinyl (e.g. —CH═CHCH₃, —CH═C(CH₃)₂ or —CH═CH₂), or allyl or methyl-substituted allyl (e.g. —CH₂CH═CH₂), isobutenyl or methyl substituted isobutenyl (e.g. ═C(CH₃)₂) and cyano; and is most preferably isobutyl;
   and:
R₄ is chosen from the group consisting of H or halogen (F, Cl, Br or I, and preferably F or Cl).

As further described herein, the invention in particular aspect relates to such a compound of Formula II that has an affinity for AT2R (measured according to the protocol set out in Example 2 below) that is better than 10 micromolar, preferably better than 1 micromolar, more preferably better than 0.1 micromolar, even more preferably better than nanomolar. In a more particular aspect, the invention relates to a compound of Formula II (as further described herein) in which: (i) the Aromatic Ring System [C], the Aliphatic Ring [A] and the acidic substituent [D] are each as further described herein; and (ii) each of the substituents R₁, R₂, R₃ and R₄ (when present) as well as the particular combination of such substituents R₁, R₂, R₃ and R₄ that is present in said compound of Formula II; and (iii) m and n (which as described herein can each independently be 0 or 1); and (iv) each of the substituents R_{A}, R_{B}, R_{C} and R_{D} (when present) as well as the particular combination of such substituents R_{A}, R_{B}, R_{C} and R_{D} that is present in said compound of Formula II; and (v) any further substituents that are present in such a compound of Formula II (e.g. on the aromatic ring system [C] and/or on the aliphatic ring system [A], as further described herein) as well as the particular combination of such substituents that is present in said compound of Formula II, are each such that (and are in combination such that) said compound of Formula II has an affinity for AT2R (measured according to the protocol set out in Example 2 below) that is better than 10 micromolar, preferably better than 1 micromolar, more preferably better than 0.1 micromolar, even more preferably better than 10 nanomolar.

In a more specific, but non-limiting aspect, the invention relates to a compound of Formula III:

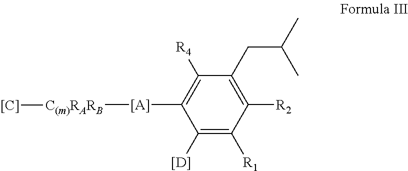

in which:
[C] is a monocyclic or polycyclic (and preferably monocyclic or bicyclic) aromatic ring system, such that: (i) the aromatic ring system contains at least one aromatic ring; (ii) the aromatic ring system is linked to the remainder of the compound of Formula III (i.e. either directly via a covalent bond or via the alkylene linking group —C₍ₘ₎R_{A}R_{B}— when said alkylene linking group is present) via a carbon atom that is present in said at least one aromatic ring; and (iii) said at least one aromatic ring (i.e. the aromatic ring that contains the carbon atom to which the remainder of the compound of Formula III is linked) preferably contains at least one (such as 1 or 2) hetero-atom(s), which hetero-atoms (when present) are preferably each independently and suitably chosen from N, S and O and are more preferably such that at least one of the hetero-atoms (when present) is a nitrogen atom; with the aromatic ring system denoted as [C] preferably being an aromatic ring or ring system that is chosen from the group consisting of the aromatic rings/ring systems of Formulae VII to LXXXIII, more preferably from the group consisting of the aromatic rings/ring systems of Formulae LIX to LXXXIII, and even more preferably from the group consisting of the aromatic rings/ring systems of Formulae LXXIII to LXXXIII;
   and:
m is 1 or 0;
   and:
each of R_{A} and R_{B}, when present, is independently chosen from hydrogen, methyl and/or trifluoromethyl, or where R_{A}+R_{B}, when present, together with the carbon atom to which they are bound form a carbonyl (C═O) group (in other words, in which R_{A}+R_{B} together are replaced by a single oxygen atom so as to form a carbonyl group with the carbon atom to which said oxygen atom is bound); and are preferably each a hydrogen atom;
   and:
[A] is either a ring system that is as schematically represented by the following Scheme C:

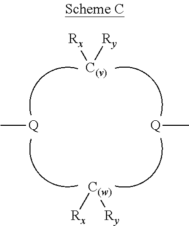

in which each atom Q is independently a carbon atom or a nitrogen atom (and preferably such that at least one atom Q is a nitrogen atom, and more preferably such that both atoms Q are nitrogen atoms), v is an integer between 1 and 4 (i.e. 1, 2, 3 or 4) and w is an integer between 1 and 4 (i.e. 1, 2, 3 or 4), such that the sum of (v+w) is 3, 4, 5, 6, 7 or 8 (and preferably 3, 4 or 5, with v and w more preferably both being 2) and such that the difference (v−w) is either 1, 0 or −1, and each Rₓ that is present and each R_{y} that is present is preferably independently chosen from the group consisting of hydrogen, methyl, ethyl, fluor (F), CF₃ and isopropyl; and in particular a ring system that is as schematically represented by the following Scheme D:

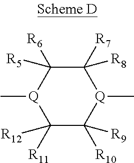

in which each atom Q is independently a carbon atom or a nitrogen atom (and preferably such that at least one atom Q is a nitrogen atom, and more preferably such that both atoms Q are nitrogen atoms) and each of R₅, R₆, R₇, R₈, R₉, R₁₀, R₁₁ and R₁₂ can independently be a suitable substituent that is preferably independently chosen from the group consisting of hydrogen, methyl, ethyl, fluor (F), CF₃ and isopropyl; or alternatively a ring system of formula XCIX or C

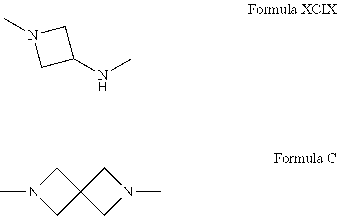

in which said ring system of Formula XCIX or Formula C may optionally be suitably substituted with one or more suitable substituents that, when present, are each preferably independently chosen from the group consisting of hydrogen, methyl, ethyl, fluor (F), CF₃ and isopropyl;
   and:
the acidic substituent denoted by [D] is chosen from the group consisting of a carboxylic acid (—COOH) group, an acylsulfonamide group (such as an acylsulfonamide group of the general formula CONHSO₂R or SO₂NHCOR wherein R is —C₁-C₈ alkyl, CH₂(C₁-C₈ cycloalkyl), CH₂(heterocyclyl with 1-6 ring atoms), C₁-C₈ alkoxy or C₁-C₈ amine), a tetrazole group or a group that is a bioisostere (as defined herein) of a tetrazole group (such as, for example and without limitation, a 4H-1,2,4-oxadiazol-5-one group), and is more preferably tetrazole or a bioisostere thereof, and most preferably tetrazole;
   and:
R₁ is chosen from the group consisting of H, halogen (F, Cl, Br or I, and preferably F or Cl), C₁-C₈ alkyl, C₃ or C₄ cycloalkyl, CF₃, C₁-C₈ alkoxy, amine (—NH₂) or C₁-C₂ substituted amine (e.g. dimethylamine or diethylamine) and cyano;
   and:
R₂ is chosen from the group consisting of H, halogen (F, Cl, Br or I, and preferably F or Cl), C₁-C₈ alkyl, C₁-C₈ alkoxy, cycloalkyl, amine (—NH₂) or C₁-C₂ substituted amine (e.g. dimethylamine or diethylamine);
   and:
R₄ is chosen from the group consisting of H or halogen (F, Cl, Br or I, and preferably F or Cl).

As further described herein, the invention in particular aspect relates to such a compound of Formula III that has an affinity for AT2R (measured according to the protocol set out in Example 2 below) that is better than 10 micromolar, preferably better than 1 micromolar, more preferably better than 0.1 micromolar, even more preferably better than 10 nanomolar. In a more particular aspect, the invention relates to a compound of Formula III (as further described herein) in which: (i) the Aromatic Ring System [C], the Aliphatic Ring [A] and the acidic substituent [D] are each as further described herein; and (ii) each of the substituents R₁, R₂ and R₄ (when present) as well as the particular combination of such substituents R₁, R₂ and R₄ that is present in said compound of Formula III; and (iii) m (which as described herein can be 0 or 1); and (iii) each of the substituents R_{A} and R_{B} (when present) as well as the particular combination of such substituents R_{A} and R_{B} that is present in said compound of Formula III; and (iv) any further substituents that are present in such a compound of Formula III (e.g. on the aromatic ring system [C] and/or on the aliphatic ring system [A], as further described herein) as well as the particular combination of such substituents that is present in said compound of Formula III, are each such that (and are in combination such that) said compound of Formula III has an affinity for AT2R (measured according to the protocol set out in Example 2 below) that is better than 10 micromolar, preferably better than 1 micromolar, more preferably better than 0.1 micromolar, even more preferably better than 10 nanomolar.

In another preferred but non-limiting aspect, the invention relates to a compound of Formula IV:

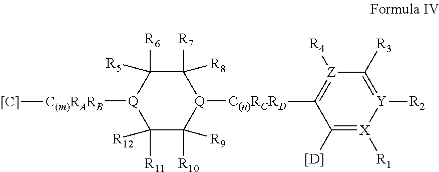

in which:
[C] is a monocyclic or polycyclic (and preferably monocyclic or bicyclic) aromatic ring system, such that: (i) the aromatic ring system contains at least one aromatic ring; (ii) the aromatic ring system is linked to the remainder of the compound of Formula IV (i.e. either directly via a covalent bond or via the alkylene linking group —C₍ₘ₎R_{A}R_{B}— when said alkylene linking group is present) via a carbon atom that is present in said at least one aromatic ring; and (iii) said at least one aromatic ring (i.e. the aromatic ring that contains the carbon atom to which the remainder of the compound of Formula IV is linked) preferably contains at least one (such as 1 or 2) hetero-atom(s), which hetero-atoms (when present) are preferably each independently and suitably chosen from N, S and O and are more preferably such that at least one of the hetero-atoms (when present) is a nitrogen atom; with the aromatic ring system denoted as [C] preferably being an aromatic ring or ring system that is chosen from the group consisting of the aromatic rings/ring systems of Formulae VII to LXXXIII, more preferably from the group consisting of the aromatic rings/ring systems of Formulae LIX to LXXXIII, and even more preferably from the group consisting of the aromatic rings/ring systems of Formulae LXXIII to LXXXIII;
   and:
m is 1 or 0 and n is 1 or 0, such that the sum of m and n is either 2, 1 or 0 and is preferably 1 or 0 (it being understood that when m=0, R_{A} and R_{B} will not be present and that when n=0, R_{C} and R_{D} will not be present), with m preferably being 1 and n preferably being 0;
   and:
each of R_{A}, R_{B}, R_{C} and R_{D}, when present, is independently chosen from hydrogen, methyl and/or trifluoromethyl, or where R_{A}+R_{B}, when present, together with the carbon atom to which they are bound form a carbonyl (C═O) group; or R_{C}+R_{D}, when present, together with the carbon atom to which they are bound form a carbonyl (C═O) group (in other words, in which R_{A}+R_{B} together are replaced by a single oxygen atom so as to form a carbonyl group with the carbon atom to which said oxygen atom is bound, or in which R_{C}+R_{D}, when present, together are replaced by a single oxygen atom so as to form a carbonyl group with the carbon atom to which said oxygen atom is bound); and are preferably each a hydrogen atom;
   and:
each Q is independently a carbon atom or a nitrogen atom, preferably such that at least one atom Q is a nitrogen atom, and more preferably such that both atoms Q are nitrogen atoms;
   and:
each of R₅, R₆, R₇, R₈, R₉, R₁₀, R₁₁ and R₁₂ can independently be a suitable substituent that is preferably independently chosen from the group consisting of hydrogen, methyl, ethyl, fluor (F), CF₃ and isopropyl;
   and:
the acidic substituent denoted by [D] is chosen from the group consisting of a carboxylic acid (—COOH) group, an acylsulfonamide group (such as an acylsulfonamide group of the general formula CONHSO₂R or SO₂NHCOR wherein R is —C₁-C₈ alkyl, CH₂(C₁-C₈ cycloalkyl), CH₂(heterocyclyl with 1-6 ring atoms), C₁-C₈ alkoxy or C₁-C₈ amine), a tetrazole group or a group that is a bioisostere (as defined herein) of a tetrazole group (such as, for example and without limitation, a 4H-1,2,4-oxadiazol-5-one group), and is more preferably tetrazole or a bioisostere thereof, and most preferably tetrazole; and:
X and Y are each either a nitrogen atom or a carbon atom, such that either both X and Y are carbon atoms or only one or X or Y is a nitrogen atom (with the other of X and Y being a carbon atom);
   and:
Z is a nitrogen atom or a carbon atom;
   and:
R₁, when present, is chosen from the group consisting of H, halogen (F, Cl, Br or I, and preferably F or Cl), C₁-C₈ alkyl, C₃ or C₄ cycloalkyl, CF₃, C₁-C₈ alkoxy, amine (—NH₂) or C₁-C₂ substituted amine (e.g. dimethylamine or diethylamine) and cyano;
   and:
R₂, when present, is chosen from the group consisting of H, halogen (F, Cl, Br or I, and preferably F or Cl), C₁-C₈ alkyl, C₁-C₈ alkoxy, cycloalkyl, amine (—NH₂) or C₁-C₂ substituted amine (e.g. dimethylamine or diethylamine);
   and:
R₃ is chosen from the group consisting of H, halogen (F, Cl, Br or I, and preferably F or Cl), C₁₋₈ alkyl (e.g. sec-butyl and in particular iso-butyl), including fluor-substituted methyl groups (e.g. trifluoromethyl and CHF₂), C₁-C₈ alkoxy, —O—CF₃, methoxyethyloxy (—O—(CH₂)₂O—CH₃) or difluoroethoxy (—O—CH₂—CHF₂), cycloalkyl (e.g. cyclopropoxy, cyclobutoxy or cyclopentoxy), —CH₂-cycloalkyl, —O—CH₂-cycloalkyl, —O-cycloalkyl, —NH— cycloalkyl, —N(C₁-C₃)-cycloalkyl, —NH-heteroalkyl, —N(C₁-C₃)-heteroalkyl, heterocyclyl (e.g. oxirane), —CH₂-heterocyclyl, —O—CH₂-heterocyclyl (e.g. —O—CH₂-oxirane), —O— heterocyclyl, —NH-heterocyclyl, —N(C₁-C₃)-heterocyclyl, vinyl or methyl-substituted vinyl (e.g. —CH═CHCH₃, —CH═C(CH₃)₂ or —CH═CH₂), or allyl or methyl-substituted allyl (e.g. —CH₂CH═CH₂), isobutenyl or methyl substituted isobutenyl (e.g. ═C(CH₃)₂) and cyano; and is most preferably isobutyl;
   and:
R₄, when present, is chosen from the group consisting of H or halogen (F, Cl, Br or I, and preferably F or Cl);
   and provided that:
X, Y and Z are chosen such (i.e. each from a carbon atom or a nitrogen atom) that the resulting ring structure form an aromatic ring (i.e. a planar conjugated ring);
   and further provided that:
when X is a nitrogen atom, R₁ is not present;
   and further provided that:
when Y is a nitrogen atom, R₂ is not present;
   and further provided that:
when Z is a nitrogen atom, R₄ is not present.

As further described herein, the invention in particular aspect relates to such a compound of Formula IV that has an affinity for AT2R (measured according to the protocol set out in Example 2 below) that is better than 10 micromolar, preferably better than 1 micromolar, more preferably better than 0.1 micromolar, even more preferably better than 10 nanomolar. In a more particular aspect, the invention relates to a compound of Formula IV (as further described herein) in which: (i) the Aromatic Ring System [C] and the acidic substituent [D] are each as further described herein; and (ii) each atom Q (which as mentioned herein can independently be a carbon atom or a nitrogen atom, with preferably at least one Q being a nitrogen atom); and (iii) each of X, Y and Z, as well as the particular combination of the atoms X, Y and Z that is present in said compound of Formula IV; and (iv) each of the substituents R₁, R₂, R₃ and R₄ (when present) as well as the particular combination of such substituents R₁, R₂, R₃ and R₄ that is present in said compound of Formula IV; and (v) each of the substituents R₅ to R₁₂ (when present) as well as the particular combination of such substituents R₅ to R₁₂ that is present in said compound of Formula IV; and (vi) m and n (which as described herein can each independently be 0 or 1); and (vii) each of the substituents R_{A}, R_{B}, R_{C} and R_{D} (when present) as well as the particular combination of such substituents R_{A}, R_{B}, R_{C} and R_{D} that is present in said compound of Formula IV; and (viii) any further substituents that are present in such a compound of Formula IV (e.g. on the aromatic ring system [C]) as well as the particular combination of such substituents that is present in said compound of Formula IV, are each such that (and are in combination such that) said compound of Formula IV has an affinity for AT2R (measured according to the protocol set out in Example 2 below) that is better than 10 micromolar, preferably better than 1 micromolar, more preferably better than 0.1 micromolar, even more preferably better than 10 nanomolar.

In a more preferred but non-limiting aspect, invention relates to a compound of Formula V:

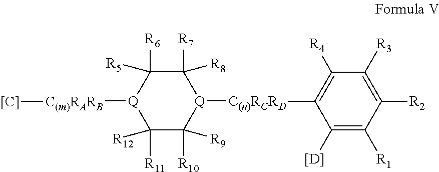

in which:
[C] is a monocyclic or polycyclic (and preferably monocyclic or bicyclic) aromatic ring system, such that: (i) the aromatic ring system contains at least one aromatic ring; (ii) the aromatic ring system is linked to the remainder of the compound of Formula V (i.e. either directly via a covalent bond or via the alkylene linking group —C₍ₘ₎R_{A}R_{B}— when said alkylene linking group is present) via a carbon atom that is present in said at least one aromatic ring; and (iii) said at least one aromatic ring (i.e. the aromatic ring that contains the carbon atom to which the remainder of the compound of Formula V is linked) preferably contains at least one (such as 1 or 2) hetero-atom(s), which hetero-atoms (when present) are preferably each independently and suitably chosen from N, S and O and are more preferably such that at least one of the hetero-atoms (when present) is a nitrogen atom; with the aromatic ring system denoted as [C] preferably being an aromatic ring or ring system that is chosen from the group consisting of the aromatic rings/ring systems of Formulae VII to LXXXIII, more preferably from the group consisting of the aromatic rings/ring systems of Formulae LIX to LXXXIII, and even more preferably from the group consisting of the aromatic rings/ring systems of Formulae LXXIII to LXXXIII;
   and:
m is 1 or 0 and n is 1 or 0, such that the sum of m and n is either 2, 1 or 0 and is preferably 1 or 0 (it being understood that when m=0, R_{A} and R_{B} will not be present and that when n=0, R_{C} and R_{D} will not be present), with m preferably being 1 and n preferably being 0;
   and:
each of R_{A}, R_{B}, R_{C} and R_{D}, when present, is independently chosen from hydrogen, methyl and/or trifluoromethyl, or where R_{A}+R_{B}, when present, together with the carbon atom to which they are bound form a carbonyl (C═O) group; or R_{C}+R_{D}, when present, together with the carbon atom to which they are bound form a carbonyl (C═O) group (in other words, in which R_{A}+R_{B} together are replaced by a single oxygen atom so as to form a carbonyl group with the carbon atom to which said oxygen atom is bound, or in which R_{C}+R_{D}, when present, together are replaced by a single oxygen atom so as to form a carbonyl group with the carbon atom to which said oxygen atom is bound), and are preferably each a hydrogen atom;
   and:
each Q is independently a carbon atom or a nitrogen atom, preferably such that at least one atom Q is a nitrogen atom, and more preferably such that both atoms Q are nitrogen atoms;
   and:
each of R₅, R₆, R₇, R₈, R₉, R₁₀, R₁₁ and R₁₂ can independently be a suitable substituent that is preferably independently chosen from the group consisting of hydrogen, methyl, ethyl, fluor (F), CF₃ and isopropyl;
   and:
the acidic substituent denoted by [D] is chosen from the group consisting of a carboxylic acid (—COOH) group, an acylsulfonamide group (such as an acylsulfonamide group of the general formula CONHSO₂R or SO₂NHCOR wherein R is —C₁-C₈ alkyl, CH₂(C₁-C₈ cycloalkyl), CH₂(heterocyclyl with 1-6 ring atoms), C₁-C₈ alkoxy or C₁-C₈ amine), a tetrazole group or a group that is a bioisostere (as defined herein) of a tetrazole group (such as, for example and without limitation, a 4H-1,2,4-oxadiazol-5-one group), and is more preferably tetrazole or a bioisostere thereof, and most preferably tetrazole;
   and:
R₁ is chosen from the group consisting of H, halogen (F, Cl, Br or I, and preferably F or Cl), C₁-C₈ alkyl, C₃ or C₄ cycloalkyl, CF₃, C₁-C₈ alkoxy, amine (—NH₂) or C₁-C₂ substituted amine (e.g. dimethylamine or diethylamine) and cyano;
   and:
R₂ is chosen from the group consisting of H, halogen (F, Cl, Br or I, and preferably F or Cl), C₁-C₈ alkyl, C₁-C₈ alkoxy, cycloalkyl, amine (—NH₂) or C₁-C₂ substituted amine (e.g. dimethylamine or diethylamine);
   and:
R₃ is chosen from the group consisting of H, halogen (F, Cl, Br or I, and preferably F or Cl), C₁₋₈ alkyl (e.g. sec-butyl and in particular iso-butyl), including fluor-substituted methyl groups (e.g. trifluoromethyl and CHF₂), C₁-C₈ alkoxy, —O—CF₃, methoxyethyloxy (—O—(CH₂)₂O—CH₃) or difluoroethoxy (—O—CH₂—CIF₂), cycloalkyl (e.g. cyclopropoxy, cyclobutoxy or cyclopentoxy), —CH₂-cycloalkyl, —O—CH₂-cycloalkyl, —O-cycloalkyl, —NH— cycloalkyl, —N(C₁-C₃)-cycloalkyl, —NH-heteroalkyl, —N(C₁-C₃)-heteroalkyl, heterocyclyl (e.g. oxirane), —CH₂-heterocyclyl, —O—CH₂-heterocyclyl (e.g. —O—CH₂-oxirane), —O— heterocyclyl, —NH-heterocyclyl, —N(C₁-C₃)-heterocyclyl, vinyl or methyl-substituted vinyl (e.g. —CH═CHCH₃, —CH═C(CH₃)₂ or —CH═CH₂), or allyl or methyl-substituted allyl (e.g. —CH₂CH═CH₂), isobutenyl or methyl substituted isobutenyl (e.g. ═C(CH₃)₂) and cyano; and is most preferably isobutyl;
   and:
R₄ is chosen from the group consisting of H or halogen (F, Cl, Br or I, and preferably F or Cl).

As further described herein, the invention in particular aspect relates to such a compound of Formula V that has an affinity for AT2R (measured according to the protocol set out in Example 2 below) that is better than 10 micromolar, preferably better than 1 micromolar, more preferably better than 0.1 micromolar, even more preferably better than 10 nanomolar. In a more particular aspect, the invention relates to a compound of Formula V (as further described herein) in which: (i) the Aromatic Ring System [C] and the acidic substituent [D] are each as further described herein; and (ii) each atom Q (which as mentioned herein can independently be a carbon atom or a nitrogen atom, with preferably at least one Q being a nitrogen atom); and (iii) each of the substituents R₁, R₂, R₃ and R₄ (when present) as well as the particular combination of such substituents R₁, R₂, R₃ and R₄ that is present in said compound of Formula V; and (v) each of the substituents R₅ to R₁₂ (when present) as well as the particular combination of such substituents R₅ to R₁₂ that is present in said compound of Formula V; and (vi) m and n (which as described herein can each independently be 0 or 1); and (vi) each of the substituents R_{A}, R_{B}, R_{C} and R_{D} (when present) as well as the particular combination of such substituents R_{A}, R_{B}, R_{C} and R_{D} that is present in said compound of Formula V; and (viii) any further substituents that are present in such a compound of Formula V (e.g. on the aromatic ring system [C]) as well as the particular combination of such substituents that is present in said compound of Formula V, are each such that (and are in combination such that) said compound of Formula V has an affinity for AT2R (measured according to the protocol set out in Example 2 below) that is better than 10 micromolar, preferably better than 1 micromolar, more preferably better than 0.1 micromolar, even more preferably better than 10 nanomolar.

In a particularly preferred but non-limiting aspect, invention relates to a compound of Formula VI:

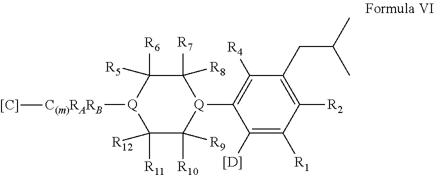

in which:
[C] is a monocyclic or polycyclic (and preferably monocyclic or bicyclic) aromatic ring system, such that: (i) the aromatic ring system contains at least one aromatic ring; (ii) the aromatic ring system is linked to the remainder of the compound of Formula VI (i.e. either directly via a covalent bond or via the alkylene linking group —C₍ₘ₎R_{A}R_{B}— when said alkylene linking group is present) via a carbon atom that is present in said at least one aromatic ring; and (iii) said at least one aromatic ring (i.e. the aromatic ring that contains the carbon atom to which the remainder of the compound of Formula VI is linked) preferably contains at least one (such as 1 or 2) hetero-atom(s), which hetero-atoms (when present) are preferably each independently and suitably chosen from N, S and O and are more preferably such that at least one of the hetero-atoms (when present) is a nitrogen atom; with the aromatic ring system denoted as [C] preferably being an aromatic ring or ring system that is chosen from the group consisting of the aromatic rings/ring systems of Formulae VII to LXXXIII, more preferably from the group consisting of the aromatic rings/ring systems of Formulae LIX to LXXXIII, and even more preferably from the group consisting of the aromatic rings/ring systems of Formulae LXXIII to LXXXIII;
   and:
m is 1 or 0;
   and:
each of R_{A} and R_{B} is independently chosen from hydrogen, methyl and/or trifluoromethyl, or where R_{A}+R_{B}, when present, together with the carbon atom to which they are bound form a carbonyl (C═O) group (in other words, in which R_{A}+R_{B} together are replaced by a single oxygen atom so as to form a carbonyl group with the carbon atom to which said oxygen atom is bound); and are preferably each a hydrogen atom;
   and:
each Q is independently a carbon atom or a nitrogen atom, preferably such that at least one atom Q is a nitrogen atom, and more preferably such that both atoms Q are nitrogen atoms;
   and:
each of R₅, R₆, R₇, R₈, R₉, R₁₀, R₁₁ and R₁₂ can independently be a suitable substituent that is preferably independently chosen from the group consisting of hydrogen, methyl, ethyl, fluor (F), CF₃ and isopropyl;
   and:
the acidic substituent denoted by [D] is chosen from the group consisting of a carboxylic acid (—COOH) group, an acylsulfonamide group (such as an acylsulfonamide group of the general formula CONHSO₂R or SO₂NHCOR wherein R is —C₁-C₈ alkyl, CH₂(C₁-C₈ cycloalkyl), CH₂(heterocyclyl with 1-6 ring atoms), C₁-C₈ alkoxy or C₁-C₈ amine), a tetrazole group or a group that is a bioisostere (as defined herein) of a tetrazole group (such as, for example and without limitation, a 4H-1,2,4-oxadiazol-5-one group), and is more preferably tetrazole or a bioisostere thereof, and most preferably tetrazole;
   and:
R₁ is chosen from the group consisting of H, halogen (F, Cl, Br or I, and preferably F or Cl), C₁-C₈ alkyl, C₃ or C₄ cycloalkyl, CF₃, C₁-C₈ alkoxy, amine (—NH₂) or C₁-C₂ substituted amine (e.g. dimethylamine or diethylamine) and cyano;
   and:
R₂ is chosen from the group consisting of H, halogen (F, Cl, Br or I, and preferably F or Cl), C₁-C₈ alkyl, C₁-C₈ alkoxy, cycloalkyl, amine (—NH₂) or C₁-C₂ substituted amine (e.g. dimethylamine or diethylamine);
   and:
R₄ is chosen from the group consisting of H or halogen (F, Cl, Br or I, and preferably F or Cl).

As further described herein, the invention in particular aspect relates to such a compound of Formula VI that has an affinity for AT2R (measured according to the protocol set out in Example 2 below) that is better than 10 micromolar, preferably better than 1 micromolar, more preferably better than 0.1 micromolar, even more preferably better than 10 nanomolar. In a more particular aspect, the invention relates to a compound of Formula VI (as further described herein) in which: (i) the Aromatic Ring System [C] and the acidic substituent [D] are each as further described herein; and (ii) each atom Q (which as mentioned herein can independently be a carbon atom or a nitrogen atom, with preferably at least one Q being a nitrogen atom); and (iii) each of the substituents R₁, R₂ and R₄ (when present) as well as the particular combination of such substituents R₁, R₂ and R₄ that is present in said compound of Formula VI; and (iv) each of the substituents R₅ to R₁₂ (when present) as well as the particular combination of such substituents R₅ to R₁₂ that is present in said compound of Formula VI; and (v) m (which as described herein can be 0 or 1); and (vi) each of the substituents R_{A} and R_{B} (when present) as well as the particular combination of such substituents R_{A} and R_{B} that is present in said compound of Formula VI; and (vii) any further substituents that are present in such a compound of Formula VI (e.g. on the aromatic ring system [C]) as well as the particular combination of such substituents that is present in said compound of Formula VI, are each such that (and are in combination such that) said compound of Formula VI has an affinity for AT2R (measured according to the protocol set out in Example 2 below) that is better than 10 micromolar, preferably better than 1 micromolar, more preferably better than 0.1 micromolar, even more preferably better than 10 nanomolar.

Some specific but non-limiting examples of compounds of the invention that are according to each of the Formulae I to VI (including such compounds that are preferred, more preferred, particularly preferred and/or most preferred), and/or that are according to the various aspects and preferred aspects of the invention that are defined herein with reference to one of the Formulae I to VI, will become clear to the skilled person based on the disclosure herein and/or are as exemplified in the Experimental Part below.

The compounds of the invention can be used in the prevention, treatment and/or management of pain, in particular chronic pain, such as the chronic pain states mentioned herein.

Thus, in a further aspect, the invention relates to the use of a compound of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein) in the prevention, treatment and/or management of pain, in particular in prevention, treatment and/or management of chronic pain.

In a further aspect, the invention relates to the use of a pharmaceutical composition that comprises a compound of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein) in the prevention, treatment and/or management of pain, in particular in prevention, treatment and/or management of chronic pain. Such a pharmaceutical composition will generally be as further described herein, and will generally comprise a pharmaceutically active amount of a compound of the invention, for example an amount that will allow the doses of the compound of the invention is mentioned herein to be administered to a subject using a suitable dosage regimen (for example as a single or multiple administrations/doses per day).

In a further aspect, the invention relates to a method for the prevention, treatment and/or management of pain, in particular chronic pain, in a subject, comprising administering to a subject (i.e. a subject in need of such prevention, treatment or management) a pharmaceutically active amount of a compound of the invention or a pharmaceutical composition comprising the same (for example, in accordance with a suitable dosage regimen as further described herein).

It will be clear to the skilled person that for the treatment of chronic pain (including the further conditions described herein that are associated with chronic pain, such as neuropathic pain and inflammatory pain) usually the compounds or compositions of the invention must be administered or used for a prolonged period of time (e.g. at least a week, such as at least a month, such as at least three months or longer, and/or until the pain is relieved or subsides), for example as part of a chronic treatment regimen or as part of an overall treatment regimen for the management of chronic pain. Such a chronic treatment regimen or pain management regimen and the use of a compound or composition of the invention as part of such a regimen can be determined by the treating physician based on the overall condition of the patient, the nature and cause of the pain and/or the particular pain state involved, and other relevant factors that will be clear to a clinician. Such treatment regimens and dosage regimens form further aspects of the invention.

The compounds and compositions of the invention can in particular be used in the prevention, treatment and/or management of pain, in particular chronic pain, where such (chronic) pain is neuropathic pain. Such neuropathic pain can be neuropathic pain that originates from the peripheral part of the nervous system (for example in the case of trigeminal or post-herpetic neuralgia, peripheral nerve injury, painful polyneuropathies, or radiculopathies) or can be neuropathic pain that originates from and/or involves the central nervous system (or example in the case of chronic neuropathic pain that develops as a result of spinal cord or brain injury, stroke or multiple sclerosis). Such uses and corresponding/associated methods of treatment form further aspects of the invention.

In a specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) can be used in the prevention, treatment and/or management of (peripheral) neuropathy. Such uses and corresponding/associated methods of treatment form further aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) can be used in the prevention, treatment and/or management of trigeminal neuralgia. Such uses and corresponding/associated methods of treatment form further aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) can be used in the prevention, treatment and/or management of chronic NP after peripheral nerve injury. Such uses and corresponding/associated methods of treatment form further aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) can be used in the prevention, treatment and/or management of painful polyneuropathy. Such uses and corresponding/associated methods of treatment form further aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) can be used in the prevention, treatment and/or management of post-herpetic neuralgia. Such uses and corresponding/associated methods of treatment form further aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) can be used in the prevention, treatment and/or management of painful radiculopathy. Such uses and corresponding/associated methods of treatment form further aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) can be used in the prevention, treatment and/or management of chronic central NP associated with spinal cord injury (SCI). Such uses and corresponding/associated methods of treatment form further aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) can be used in the prevention, treatment and/or management of chronic central NP associated with brain injury. Such uses and corresponding/associated methods of treatment form further aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) can be used in the prevention, treatment and/or management of chronic central post-stroke pain. Such uses and corresponding/associated methods of treatment form further aspects of the invention.

However, it should generally be noted that, without being limited to any specific mechanism or hypothesis, it is expected that the compounds of the invention will likely be more efficacious in preventing, treating or managing types of neuropathic pain that are generally associated with the peripheral nervous system (i.e. compared to types of neuropathic pain that are generally associated with the central nervous system), so that the use of the compounds of the invention in the prevention, treatment and/or management of the types of neuropathic pain that are associated with and/or arise out of the peripheral nervous system (e.g. caused by disease, disfunction or damage of/to peripheral nerves) will generally form preferred applications and aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) can be used in the prevention, treatment and/or management of chronic central NP associated with multiple sclerosis. Such uses and corresponding/associated methods of treatment form further aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) can be used in the prevention, treatment and/or management of inflammatory pain. Such uses and corresponding/associated methods of treatment form further aspects of the invention.

In the context of the invention, prevention, treatment and/or management of pain (including the various types of pain mentioned herein) also includes reducing, alleviating and/or relieving pain and/or the intensity, severity and/or duration of pain; reducing, alleviating and/or relieving sensitivity or hypersensitivity to pain; reducing, alleviating and/or relieving hyperalgesia; and/or reducing, alleviating and/or relieving allodynia, as well as delaying the onset of pain.

In a further aspect, the invention relates to methods for the prevention, treatment and/or management of a chronic neuropathic pain that originates from the peripheral part of the nervous system in a subject, which method comprises administering to a subject (i.e. a subject in need of such prevention, treatment or management) a pharmaceutically active amount of a compound of the invention or a pharmaceutical composition comprising the same (for example, in accordance with a suitable dosage regimen as further described herein).

In a further aspect, the invention relates to methods for the prevention, treatment and/or management of a chronic neuropathic pain that originates from and/or involve the central nervous system that originates from the peripheral part of the nervous system in a subject, which method comprises administering to a subject (i.e. a subject in need of such prevention, treatment or management) a pharmaceutically active amount of a compound of the invention or a pharmaceutical composition comprising the same (for example, in accordance with a suitable dosage regimen as further described herein).

In a further aspect, the invention relates to methods for the prevention, treatment and/or management of peripheral neuropathy in a subject, comprising administering to a subject (i.e. a subject in need of such prevention, treatment or management) a pharmaceutically active amount of a compound of the invention or a pharmaceutical composition comprising the same (for example, in accordance with a suitable dosage regimen as further described herein).

In a further aspect, the invention relates to methods for the prevention, treatment and/or management of diabetic neuropathy in a subject, comprising administering to a subject (i.e. a subject in need of such prevention, treatment or management) a pharmaceutically active amount of a compound of the invention or a pharmaceutical composition comprising the same (for example, in accordance with a suitable dosage regimen as further described herein).

In a further aspect, the invention relates to methods for the prevention, treatment and/or management of pain, in particular chronic pain, comprising administering to a subject (i.e. a subject in need of such prevention, treatment or management) a pharmaceutically active amount of a compound of the invention or a pharmaceutical composition comprising the same (for example, in accordance with a suitable dosage regimen as further described herein), in which said (chronic) pain is one of the following pain states: trigeminal or post-herpetic neuralgia, peripheral nerve injury, painful polyneuropathy, painful radiculopathy, chronic neuropathic pain that develops as a result of spinal cord or brain injury, stroke or multiple sclerosis; chronic NP after peripheral nerve injury, chronic central NP associated with spinal cord injury (SCI), chronic central NP associated with brain injury, chronic central post-stroke pain, chronic central NP associated with multiple sclerosis, and/or chronic pain resulting from tissue injury (also including, without limitation, chronic pain following surgery and phantom pain following amputation).

Again, it will be clear to the skilled person that for the treatment of any of the chronic pain states mentioned herein, usually the compounds or compositions of the invention must be administered or used for a prolonged period of time (e.g. at least a week, such as at least a month, such as at least three months or longer, and/or until the pain is relieved or subsides), for example as part of a chronic treatment regimen or as part of an overall treatment regimen for the management of chronic pain. Such a chronic treatment regimen or pain management regimen and the use of a compound or composition of the invention as part of such a regimen can be determined by the treating physician based on the overall condition of the patient, the nature and cause of the pain and/or the particular pain state involved, and other relevant factors that will be clear to a clinician. Such treatment regimens and dosage regimens form further aspects of the invention.

It will again also be clear that, the context of the various pain states mentioned herein, prevention, treatment and/or management of pain of these pain states also includes reducing, alleviating and/or relieving pain and/or the intensity, severity and/or duration of pain; reducing, alleviating and/or relieving sensitivity or hypersensitivity to pain; reducing, alleviating and/or relieving hyperalgesia; and/or reducing, alleviating and/or relieving allodynia, as well as delaying the onset of pain.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) are used for reducing, alleviating and/or relieving hyperalgesia, in particular hyperalgesia that is associated with chronic pain, more in particular hyperalgesia that is associated with neuropathic pain and/or inflammatory pain, such as hyperalgesia that is associated with one of the specific pain states mentioned herein. Such uses and corresponding/associated methods of treatment form further aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) are used for reducing, alleviating and/or relieving allodynia, in particular allodynia that is associated with chronic pain, more in particular allodynia that is associated with neuropathic pain and/or inflammatory pain, such as allodynia that is associated with one of the specific pain states mentioned herein. Such uses and corresponding/associated methods of treatment form further aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) are used for reducing, alleviating and/or relieving inflammatory pain, such as hypersensitivity to pain that occurs in response to tissue damage and inflammation. Such uses and corresponding/associated methods of treatment form further aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) are used for reducing, alleviating and/or relieving pain, and in particular inflammatory pain, that is caused by and/or associated with trauma and/or arthritis. Such uses and corresponding/associated methods of treatment form further aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) are used for reducing, alleviating and/or relieving post-operative pain. Such uses and corresponding/associated methods of treatment form further aspects of the invention.

Again, it will be clear to the skilled person that for the prevention, treatment and/or management of any of the aforementioned pain states, the compounds or compositions of the invention must be administered or used according to a suitable treatment regimen and/or as an overall treatment regimen for the management of the pain state involved (which may for example be applied until the pain subsides or is reduced and/or the underlying disease or condition is treated or reduced, or may be chronic in case of a chronic disease). Such a treatment regimen or pain management regimen and the use of a compound or composition of the invention as part of such a regimen can be determined by the treating physician based on the overall condition of the patient, the nature and cause of the pain and/or the particular pain state involved, and other relevant factors that will be clear to a clinician. Such treatment regimens and dosage regimens form further aspects of the invention.

It will also be clear to the skilled person that, as part of the use of a compound or composition of the invention in the prevention, treatment and/or management of any of the types of pain and/or pain states mentioned herein, the use of a compound or composition of the invention may be suitably combined with the administration or use of one or more other compounds or active substances known per se for the prevention, treatment and/or management of pain (such as chronic pain), and in particular be suitably combined with the administration or use of one or more other compounds or active substances known per se for the prevention, treatment and/or management of the particular type of pain or pain state involved. Such compounds and active substances will be clear to the skilled person based on the disclosure herein, and the use of the compound of the invention in combination with such other compounds and active substances as part of such a treatment regimen can be determined by the treating physician based on the overall condition of the patient, the nature and cause of the pain and/or the particular pain state involved, and other relevant factors that will be clear to a clinician. Such treatment regimens and dosage regimens form further aspects of the invention.

Again, as further described herein, when a compound of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein) is present in a pharmaceutical composition (as described herein) and/or is used for a therapeutic use (as further described herein) and/or as part of a method-of-treatment (as further described herein), such a compound of the invention preferably has an affinity for AT2R (measured according to the protocol set out in Example 2 below) that is better than 10 micromolar, more preferably better than 1 micromolar, even more preferably better than 0.1 micromolar, and may in particular have an affinity for AT2R that is better than 10 nanomolar.

As mentioned herein, it is also expected that the compounds of the invention can be used in the prevention and treatment of diseases and disorders that can be prevented or treated by modulating, in a subject in need thereof, the angiotensin II receptor type 2, AT2R-mediated signaling and/or the pathways and/or biological processes in which AT2R and/or AT2R-mediated signaling is involved. It is further expected that the compounds and composition can be used for the prevention and treatment of diseases and disorders that can be prevented or treated by administering, to a subject in need thereof, a compound that is capable of competing with the binding of one or more natural ligands to the angiotensin II receptor type 2.

Such diseases include, but are not limited to those diseases that are mentioned in the art as being associated with AT2R and/or AT2R-mediated signaling and other diseases and disorders for which the use of known modulators (and in particular modulators that compete for ligand binding to AT2R) and/or the use of known inhibitors and/or antagonists of AT2R and/or AT2R-mediated signaling have been described in the art. Reference is for example made to the diseases and disorders listed in WO 2019/179515 (listed in more detail below).

Thus, in another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) are used in the prevention and/or treatment of diseases and disorders that can be prevented or treated by modulating, in a subject in need thereof, the angiotensin II receptor type 2, AT2R-mediated signaling and/or the pathways and/or biological processes in which AT2R and/or AT2R-mediated signaling, in particular by administering, to said subject, one or more pharmaceutically active amounts (e.g. doses) of a compound of the invention, according to a suitable treatment or dosage regimen (which can be determined by the treating physician based on the state of the patient, the nature of the disease involved, the severity of the disease and/or its symptoms, and other factors that can be determined and suitably taken into account by the treating physician). Such uses and corresponding/associated methods of treatment, as well as such treatment regimens and dosage regimens form further aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) are used in the prevention and/or treatment of diseases and disorders that can be prevented or treated by administering, to a subject in need thereof, a compound that is capable of competing with the binding of one or more natural ligands to the angiotensin II receptor, in particular by administering, to said subject, one or more pharmaceutically active amounts (e.g. doses) of a compound of the invention, according to a suitable treatment or dosage regimen (which can be determined by the treating physician based on the state of the patient, the nature of the disease involved, the severity of the disease and/or its symptoms, and other factors that can be determined and suitably taken into account by the treating physician).

Such uses and corresponding/associated methods of treatment, as well as such treatment regimens and dosage regimens form further aspects of the invention.

In another specific, but non-limiting aspect, compounds and compositions of the invention (such as a compound of Formula I as defined herein, in particular a compound of Formula II as defined herein, more in particular a compound of Formula III as defined herein, and preferably a compound of Formula IV as defined herein, more preferably a compound of Formula V as defined herein, and even more preferably a compound of Formula VI as defined herein, or a pharmaceutical composition comprising the same) are used in the prevention and/or treatment of one of the following diseases and disorders (which are mentioned in for example WO 2019/179515 as being AT2R-mediated disorders):
a cerebrovascular disorder (which includes cerebral vasospasm and cerebral ischemia); cognitive dysfunction (which includes amnesia, senile dementia, AIDS related Dementia and Down syndrome); central nervous system diseases or conditions (including addiction such as alcoholism, anxiety, depression or dysthymia, epilepsy, hyperactivity, pain, Parkinson's disease, mental illness, sleep disorders, irregularities autonomic function, and tardive dyskinesia, schizophrenia, demyelinating diseases such as multiple sclerosis and amyotrophic lateral sclerosis; respiratory diseases (including bronchospasm, asthma and chronic obstructive airway disease): neuronal tumor; inflammatory disease (including inflammatory bowel disease and osteoarthritis); gastrointestinal (GI) disease or condition (including ulcerative colitis, Crohn's disease and incontinence); vasodilation caused by blood flow disorders; allergic diseases (including allergies such as eczema, rhinitis and contact dermatitis); vasospasm (including angina, migraine and Raynaud's disease); fiber and collagen disease (including scleroderma and eosinophilic schistosomiasis; reflex sympathetic dystrophy (including shoulder-hand syndrome); stress-related somatic disorders; peripheral neuropathy; neuralgia; autoimmune disease (including systemic erythema) Lupus, rheumatoid arthritis, psoriasis and graft versus host disease; and rheumatic diseases including fibrositis.
neuropathic disorders (including primary neuropathy and secondary neuropathy, such as peripheral neuropathy) or symptoms associated with it (including hyperesthesia, hyperalgesia, allodynia, spontaneous burning, numbness, weakness, burning pain), shooting pain and loss of reflexes, preferably neuropathic pain; wherein the secondary neuropathy includes: diabetic neuropathy; herpes zoster-associated neuropathy; uremia-associated neuropathy; amyloidosis neuropathy; neuropathy; hereditary motor and sensory neuropathy; hereditary sensory neuropathy; hereditary sensory and autonomic neuropathy; hereditary neuropathy with ulcer damage; nitrofurantoin neuropathy; sausage-like swelling neuropathy; neuropathy caused by nutritional deficiencies; neuropathy and complex regional pain syndrome; neuropathy caused by repetitive activities (such as typing or working on assembly lines); neuropathy caused by antiretroviral drugs (such as zalcitabine and didanosine), antibiotics (such as nitrazole and isoniazid), gold compounds, chemotherapeutic drugs (e.g. vincristine), alcohol, lead; peripheral neuropathy caused by arsenic, mercury, and organophosphate insecticides; peripheral neuropathy associated with the infection process (e.g. Guillain-Barre syndrome);
a condition characterized by neuronal hypersensitivity, including hyperalgesia conditions, such as fibromyalgia, irritable bowel syndrome;
a disorder associated with abnormal nerve regeneration, including neuronal hypersensitivity, breast pain, interstitial cystitis, vulvar pain, cancer-induced neuropathy;
inflammatory pain, which can be attributed to conditions characterized by inflammation (including burns such as chemistry, friction or thermal burns; autoimmune diseases such as rheumatoid arthritis; inflammatory bowel diseases such as Crohn's disease and Colitis; osteoarthritis, carditis, dermatitis, myositis, neuritis and collagen vascular disease);
impaired nerve conduction velocity, which may be associated with neuropathic disorders (such as peripheral neuropathy) as described above, as well as carpal tunnel syndrome, ulnar neuropathy, Guillain-Barre syndrome, facial scapula muscle atrophy, and disc herniation;
cell proliferative disorders, including cancer (including leukemia, melanoma, prostate cancer, breast cancer, ovarian cancer, basal cell carcinoma, squamous cell carcinoma, sarcoma, fibrosarcoma, colon cancer, lung cancer); and non-cancerous hyperplasia symptoms (including skin conditions such as sputum, keloids, psoriasis, sputum disorders, and scar tissue reduction and cosmetic remodeling);
conditions associated with imbalance between bone resorption and bone formation, including osteoporosis;
which method comprises administering, to said subject, one or more pharmaceutically active amounts (e.g. doses) of a compound of the invention, according to a suitable treatment or dosage regimen (which can be determined by the treating physician based on the state of the patient, the nature of the disease involved, the severity of the disease and/or its symptoms, and other factors that can be determined and suitably taken into account by the treating physician).

In a further aspect, the invention relates to methods for the prevention, treatment and/or management of diabetes, and in particular type II diabetes, in a subject, comprising administering to a subject (i.e. a subject in need of such prevention, treatment or management) a pharmaceutically active amount of a compound of the invention or a pharmaceutical composition comprising the same (for example, in accordance with a suitable dosage regimen as further described herein).

The compounds of the invention may be prepared in a manner known per se, for example as known per se for structurally related compounds, using well-known techniques of organic chemistry. Some specific but non-limiting methods are illustrated in the Experimental Part below.

It will also be clear that when the desired compounds of the invention, and/or the starting materials, precursors and/or intermediates used in the preparation thereof, contain functional groups that are sensitive to the reaction conditions used in the preparation of the compounds of the invention (i.e. that would undergo undesired reactions under those conditions if they were not suitably protected) can be protected during said reaction with a suitable protective group, which protective group can then be suitably removed after either completion of said reaction and/or as a later or final step in the preparation of the compounds of the invention. Suitable protective groups, as well as methods and conditions for inserting them and removing them, will be clear to the skilled person and are generally described in the standard handbooks of organic chemistry, such as Greene and Wuts, “Protective groups in organic synthesis”, 3^{rd} Edition, Wiley and Sons, 1999. It will also be clear to the skilled person that compounds of the invention in which one or more functional groups have been protected with suitable functional groups can find use as intermediates in the production and/or synthesis of the compounds of the invention, and as such form a further aspect of the invention.

For pharmaceutical use, the compounds of the invention may be used as a free acid or base, and/or in the form of a pharmaceutically acceptable acid-addition and/or base-addition salt (e.g. obtained with non-toxic organic or inorganic acid or base), in the form of a hydrate, solvate and/or complex, and/or in the form or a pro-drug or pre-drug, such as an ester. Such salts, hydrates, solvates, etc. and the preparation thereof will be clear to the skilled person; reference is for instance made to the salts, hydrates, solvates, etc. described in U.S. Pat. Nos. 6,372,778, 6,369,086, 6,369,087 and 6,372,733.

Generally, for pharmaceutical use, the compounds of the inventions may be formulated as a pharmaceutical preparation comprising at least one compound of the invention and at least one pharmaceutically acceptable carrier, diluent or excipient and/or adjuvant, and optionally one or more further pharmaceutically active compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is again made to for instance U.S. Pat. Nos. 6,372,778, 6,369,086, 6,369,087 and 6,372,733, as well as to the standard handbooks, such as the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other pharmaceutically active substances (which may or may not lead to a synergistic effect with the compounds of the invention) and other substances that are commonly used in pharmaceutical formulations, such as lubricating agents, wetting agents, emulsifying and suspending agents, dispersing agents, disintegrants, bulking agents, fillers, preserving agents, sweetening agents, flavoring agents, flow regulators, release agents, etc. The compositions may also be formulated so as to provide rapid, sustained or delayed release of the active compound(s) contained therein, for example using liposomes or hydrophilic polymeric matrices based on natural gels or synthetic polymers.

The above preparations may be prepared in a manner known per se, which usually involves mixing the active substance(s) to be used with the one or more pharmaceutically acceptable carriers, necessary under aseptic conditions. Reference is again made to U.S. Pat. Nos. 6,372,778, 6,369,086, 6,369,087 and 6,372,733 and the further prior art mentioned above, as well as to the standard handbooks, such as the latest edition of Remington's Pharmaceutical Sciences.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the invention, e.g. about 10, 25, 50, 100, 200, 300 or 400 mg per unit dosage.

The compounds can be administered by a variety of routes including the oral, rectal, transdermal, subcutaneous, intravenous, intramuscular or intranasal routes, depending mainly on the specific preparation used and the condition to be treated or prevented, and with oral and intravenous administration usually being preferred. The at least one compound of the invention will generally be administered in an “effective amount”, by which is meant any amount of a compound of the Formulas I or VI above that, upon suitable administration, is sufficient to achieve the desired therapeutic or prophylactic effect in the individual to which it is administered. Usually, depending on the condition to be prevented or treated and the route of administration, such an effective amount will usually be between 0.01 to 1000 mg, more often between 0.1 and 500 mg, such as between 1 and 250 mg, for example about 5, 10, 20, 50, 100, 150, 200 or 250 mg, per kilogram body weight of the patient per day, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. Nos. 6,372,778, 6,369,086, 6,369,087 and 6,372,733 and the further prior art mentioned above, as well as to the standard handbooks, such as the latest edition of Remington's Pharmaceutical Sciences.

Thus, in a further aspect, the invention relates to a composition, and in particular a composition for pharmaceutical use, that contains at least one compound of the invention and at least one suitable carrier (i.e. a carrier suitable for pharmaceutical use). The invention also relates to the use of a compound of the invention in the preparation of such a composition.

It is also envisaged that the above compounds and compositions may be of value in the veterinary field, which for the purposes herein not only includes the prevention and/or treatment of diseases in animals, but also—for economically important animals such as cattle, pigs, sheep, chicken, fish, etc.—enhancing the growth and/or weight of the animal and/or the amount and/or the quality of the meat or other products obtained from the animal.

Thus, in a further aspect, the invention relates to a composition for veterinary use that contains at least one compound of the invention and at least one suitable carrier (i.e. a carrier suitable for veterinary use). The invention also relates to the use of a compound of the invention in the preparation of such a composition.

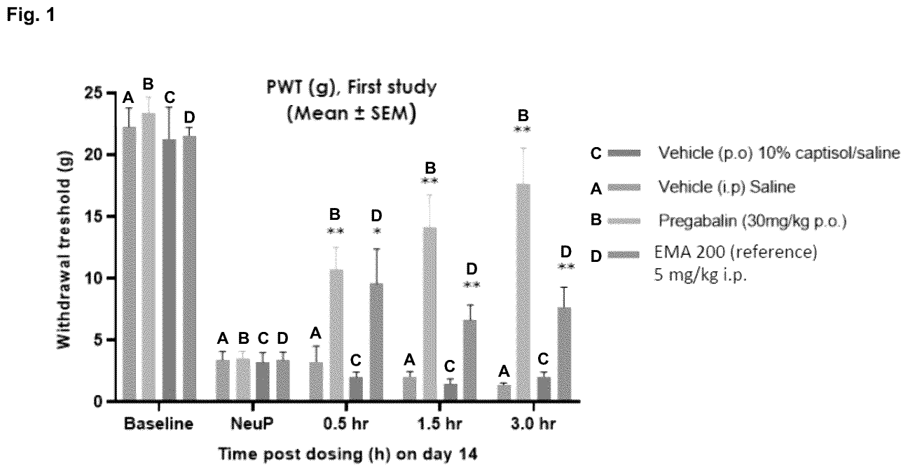

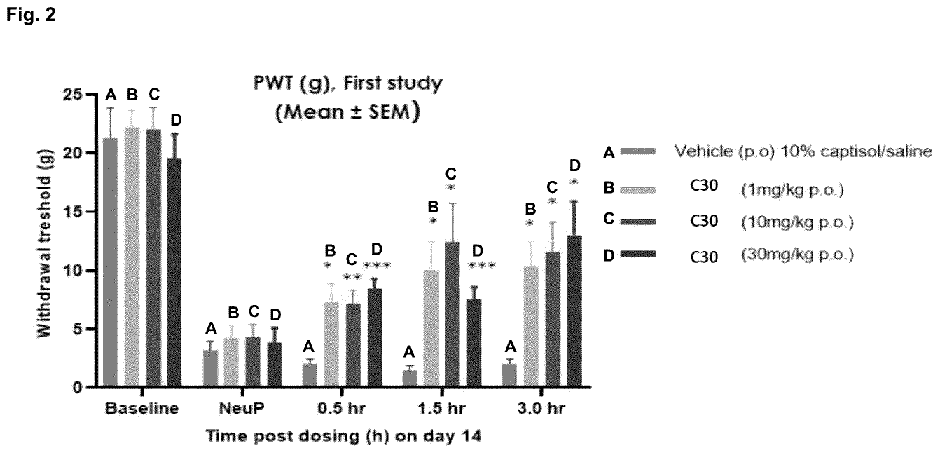

The invention will now be illustrated by means of the experimental part below and FIGS. 1 and 2, which are graphs showing results obtained in the CCI model that is used in Example 3). These experimental part and figures do not limit the scope of the invention in any way.

### EXPERIMENTAL PART

### Example 1: Synthetic Procedures

### Synthesis of Intermediates

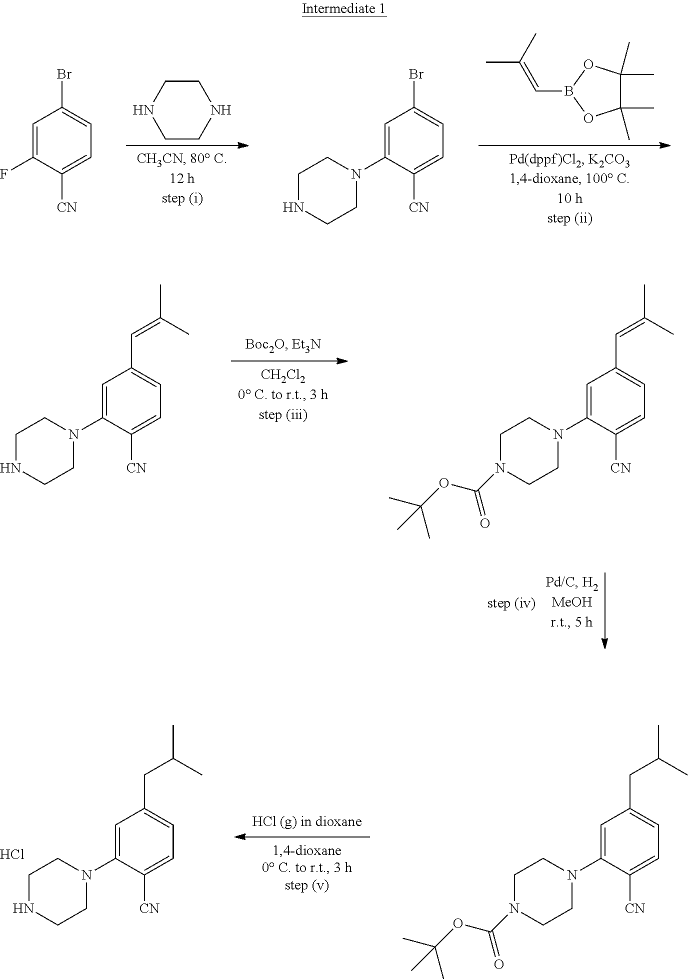

### Step (i): 4-Bromo-2-(piperazin-1-yl)benzonitrile

After addition of piperazine (107.5 g, 1250.0 mmol) to a stirred solution of 4-bromo-2-fluorobenzonitrile (50 g, 250.0 mmol) in acetonitrile (500 mL), the reaction was continued at 80° C. for 12 h. After the completion of the reaction was confirmed by TLC, the reaction mixture was evaporated to dryness, water was added and the obtained solution was extracted with EtOAc (3×500 mL). The combined organic layers were washed with sat. brine, dried over anhydrous sodium sulfate and evaporated under reduced pressure. The crude gummy solid (57.0 g, crude) was taken to the next step without purification.

### Step (ii): 4-(2-Methylprop-1-en-1-yl)-2-(piperazin-1-yl)benzonitrile

To a stirred solution of 4-bromo-2-(piperazin-1-yl)benzonitrile (57.0 g, 214.2 mmol) in 1,4-dioxane (500 mL) was added 4,4,5,5-tetramethyl-2-(2-methylprop-1-en-1-yl)-1,3,2-dioxaborolane (46.76 g, 257.0 mmol), followed by K₂CO₃ (73.89 g, 535.4 mmol) and the resultant mixture was bubbled with argon for 20 min. Then Pd(dppf)Cl₂ (15.7 g, 21.4 mmol) was added after which the reaction was heated to 100° C. for 10 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure afforded 4-(2-methylprop-1-en-1-yl)-2-(piperazin-1-yl)benzonitrile (47.0 g) which was taken to the next step without additional purification.

### Step (iii): tert-Butyl 4-(2-cyano-5-(2-methylprop-1-en-1-yl)phenyl)piperazine-1-carboxylate

To a stirred solution of 4-(2-methylprop-1-en-1-yl)-2-(piperazin-1-yl)benzonitrile (47 g, 194.8 mmol) in CH₂Cl₂ (300 mL) at 0° C. was added Et₃N (81.0 mL, 584.3 mmol). The solution was stirred at 0° C. for 10 minutes, after which Boc-anhydride (63.8 g, 292.5 mmol) was added and the reaction was allowed to continue stirring at room temperature for an additional 3 hours. After completion of the reaction was confirmed by TLC, the reaction mixture was diluted with cold water and extraction with CH₂Cl₂ (3×300 mL) was performed. The combined organic layers were washed with sat. brine, dried over anhydrous sodium sulfate and evaporated under reduced pressure. The crude obtained was purified by column chromatography over silica gel (20-30% EtOAc in hexane) to afford tert-butyl 4-(2-cyano-5-(2-methylprop-1-en-1-yl)phenyl)piperazine-1-carboxylate as a white solid (53.2 g, 80%).

### Step (iv): tert-Butyl 4-(2-cyano-5-isobutylphenyl)piperazine-1-carboxylate

A stirred solution of tert-butyl 4-(2-cyano-5-(2-methylprop-1-en-1-yl)phenyl)piperazine-1-carboxylate (52 g, 19.4 mmol) in MeOH (200 mL) was hydrogenated over 10% Pd/C (5.2 g) under 5 Kg/cm² H₂ pressure using a Parr hydrogenator for 5 h at ambient temperature. After confirming the completion of reaction by LC-MS, the reaction mixture was filtered through a Celite® bed and was evaporated in vacuo. The crude residue (47.5 g) was taken to the next step without further purification.

### Step (v): 4-Isobutyl-2-(piperazin-1-yl)benzonitrile hydrochloride

To the stirred solution of tert-butyl 4-(2-cyano-5-isobutylphenyl)piperazine-1-carboxylate (47.5 g, 138.5 mmol) in 1,4-dioxane (50 mL) was added HCl (g) in dioxane (150 mL) at 0° C., after which the solution was slowly warmed up to room temperature and continued stirring at r.t. for 3 h. After the completion of the reaction was confirmed by TLC, the reaction mixture was evaporated to dryness under reduced pressure. The crude residue was further triturated with hexane to afford 4-isobutyl-2-(piperazin-1-yl)benzonitrile hydrochloride (35.0 g).

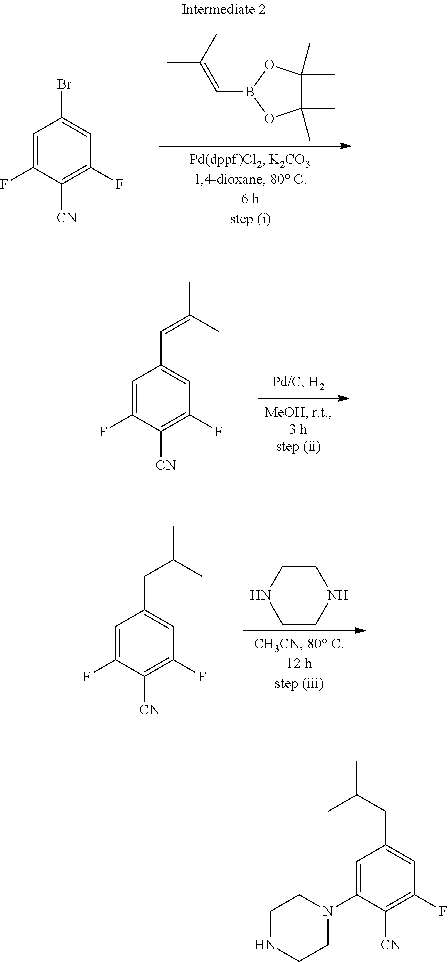 [SMILES: N#Cc1c(F)cc(Br)cc1F ; CC(C)=CB1OC(C)(C)C(C)(C)O1 ; CC(C)=Cc1cc(F)c(C#N)c(F)c1 ; CC(C)Cc1cc(F)c(C#N)c(F)c1 ; C1CNCCN1 ; CC(C)Cc1cc(F)c(C#N)c(N2CCNCC2)c1]

### Step (i): 2,6-Difluoro-4-(2-methylprop-1-en-1-yl)benzonitrile

To a stirred solution of 4-bromo-2,6-difluorobenzonitrile (5.0 g, 22.93 mmol) in 1,4-dioxane (100 mL) was added 4,4,5,5-tetramethyl-2-(2-methylprop-1-en-1-yl)-1,3,2-dioxaborolane (5.01 g, 27.52 mmol), followed by K₂CO₃ (7.92 g, 57.34 mmol) and the resultant mixture was bubbled with argon for 20 min. Then Pd(dppf)Cl₂ (0.935 g, 1.156 mmol) was added after which the reaction was heated to 80° C. for 6 h. After the completion of reaction was confirmed by TLC, the reaction mixture was evaporated under reduced pressure to remove the volatiles. The residue was dissolved with ethyl acetate and washed with water, sat. brine, dried over anhydrous sodium sulfate and evaporated under reduced pressure to afford a crude residue. The crude compound thus obtained was purified by column chromatography over silica gel eluting with 15-20% EtOAc in hexane to afford 2,6-difluoro-4-(2-methylprop-1-en-1-yl)benzonitrile as colorless gum (4.0 g, 90%).

### Step (ii): 2,6-Difluoro-4-isobutylbenzonitrile

A stirred solution of 2,6-difluoro-4-(2-methylprop-1-en-1-yl)benzonitrile (4 g, 20.72 mmol) in MeOH was hydrogenated over 10% Pd/C (0.4 g) under 5 Kg/cm² H₂ pressure using a Parr hydrogenator for 3 h at ambient temperature. After confirming the completion of reaction by LC-MS, the reaction mixture was filtered through a Celite® bed and was evaporated in vacuo. The crude residue was purified by silica chromatography using 20-25% EtOAc in hexane to afford 2,6-difluoro-4-isobutylbenzonitrile as a colorless gum (3.3 g, 82%).

### Step (iii): 2-Fluoro-4-isobutyl-6-(piperazin-1-yl)benzonitrile

To a stirred solution of 2,6-difluoro-4-isobutylbenzonitrile (2 g, 10.25 mmol) in acetonitrile (25 mL) was added piperazine (2.20 g, 25.64 mmol) after which the reaction was kept at 80° C. for 12 h. After the completion of the reaction was confirmed by TLC, the reaction mixture was evaporated to dryness, water was added and the obtained solution was extracted with EtOAc (2×100 mL). The combined organic layers were washed with sat. brine, dried over anhydrous sodium sulfate and evaporated under reduced pressure. The crude residue obtained as an off-white solid (2.40 g, 95%) was used as such in the following steps.

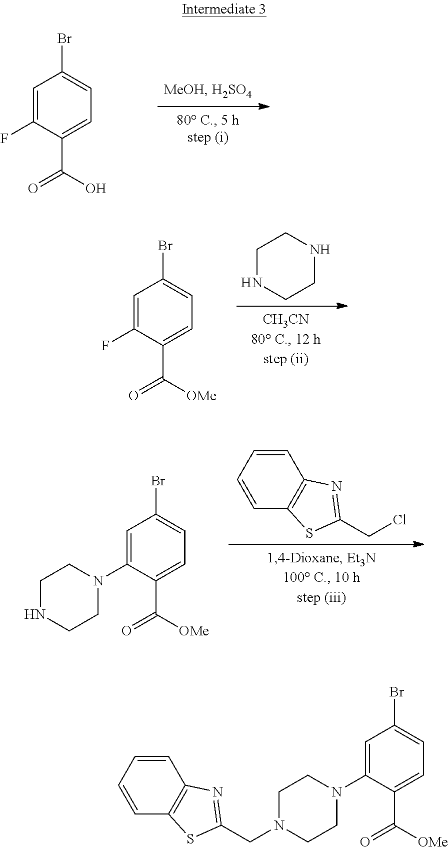 [SMILES: O=C(O)c1ccc(Br)cc1F ; COC(=O)c1ccc(Br)cc1F ; C1CNCCN1 ; COC(=O)c1ccc(Br)cc1N1CCNCC1 ; ClCc1nc2ccccc2s1 ; COC(=O)c1ccc(Br)cc1N1CCN(Cc2nc3ccccc3s2)CC1]

### Step (i): Methyl 4-bromo-2-fluorobenzoate

To a stirred solution of 4-bromo-2-fluorobenzoic acid (5.0 g, 22.83 mmol) in MeOH (50 mL) was added H₂SO₄ (25 mL) at 0° C., after which the reaction was brought to 80° C. and kept stirring for 5 h. After the completion of the reaction was confirmed by TLC, the reaction mixture was evaporated to dryness, water was added, and the obtained solution was treated with sat. NaHCO₃ and extracted with ethyl acetate (3×100 mL). The combined organic layers were washed with water, sat. brine and dried over anhydrous sodium sulfate. Evaporation under reduced pressure afforded a crude residue (4.93 g crude) which was taken for the next step without additional purification.

### Step (ii): Methyl 4-bromo-2-(piperazin-1-yl)benzoate

To a stirred solution of methyl 4-bromo-2-fluorobenzoate (4.8 g, 20.6 mmol) in acetonitrile (50 mL) was added piperazine (8.86 g, 103.0 mmol), after which the resulting solution was stirred at 80° C. for 12 h. After completion of the reaction was confirmed by TLC, the reaction mixture was evaporated to dryness, water was added and the obtained solution was extracted with EtOAc (3×100 mL). The combined organic layers were washed with sat. brine, dried over anhydrous sodium sulfate and evaporated in vacuo to afford methyl 4-bromo-2-(piperazin-1-yl)benzoate (5.33 g crude) which was used as such in the next step.

### Step (iii): Methyl 2-(4-(benzo[d]thiazol-2-ylmethyl)piperazin-1-yl)-4-bromobenzoate

To a stirred solution of methyl 4-bromo-2-(piperazin-1-yl)benzoate (5.0 g, 16.72 mmol) in 1,4-dioxane (50 mL) was added Et₃N (6.95 mL, 50.15 mmol) at 0° C., followed after 10 minutes by the addition of 2-(chloromethyl)benzo[d]thiazole (3.65 g, 20.06 mmol). The resulting reaction mixture was stirred at 100° C. for 10 h. After completion of the reaction was confirmed by TLC, cold water was added to the reaction mixture which was then extracted with CH₂Cl₂ (3×100 mL). The combined organic layers were washed with sat. brine, dried over anhydrous sodium sulfate and concentrated in vacuo to afford a crude residue, which was further purified by column chromatography over silica gel (35-45% EtOAc in hexane), yielding the envisaged compound methyl 2-(4-(benzo[d]thiazol-2-ylmethyl)piperazin-1-yl)-4-bromobenzoate (5.17 g, 69%).

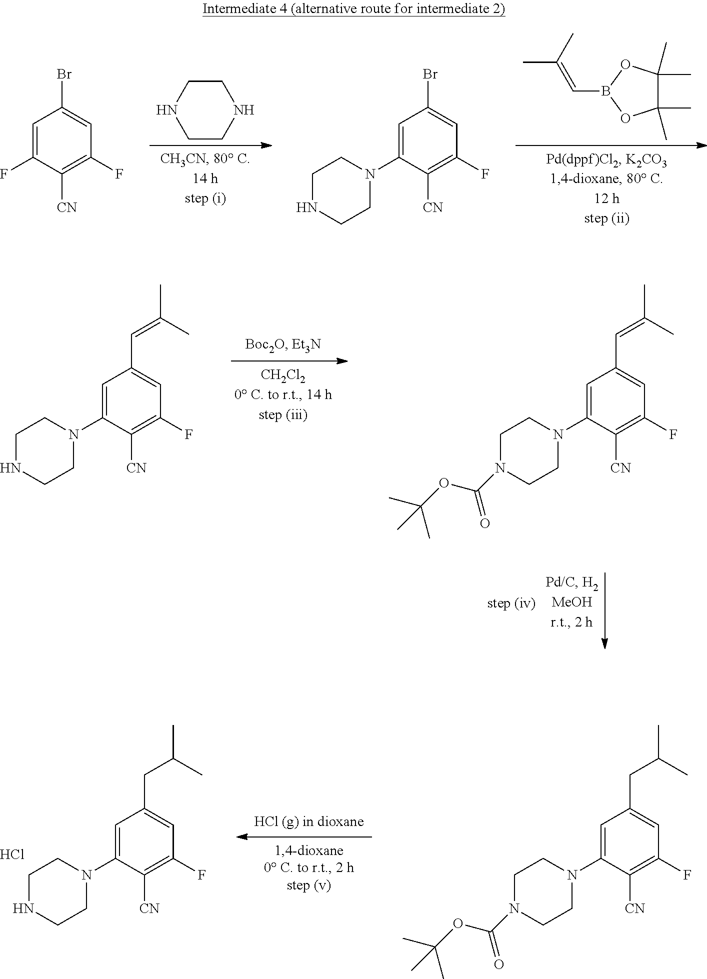

### Step (i): 4-Bromo-2-fluoro-6-(piperazin-1-yl)benzonitrile

After addition of piperazine (0.986 g, 11.4 mmol) to a stirred solution of 4-bromo-2,6-difluorobenzonitrile (0.5 g, 2.29 mmol) in acetonitrile (20 mL), the reaction was continued at 80° C. for 14 h. After the completion of the reaction was confirmed by TLC, the reaction mixture was evaporated to dryness. Next, the crude residue was purified by column chromatography over silica gel, eluting using 24% of MeOH in CH₂Cl₂, to afford 4-bromo-2-fluoro-6-(piperazin-1-yl)benzonitrile as gummy liquid (0.51 g, 78%).

### Step (ii): 2-Fluoro-4-(2-methylprop-1-en-1-yl)-6-(piperazin-1-yl)benzonitrile

To a stirred solution of 4-bromo-2-fluoro-6-(piperazin-1-yl)benzonitrile (0.5 g, 1.76 mmol) in 1,4-dioxane (20 mL) was added 4,4,5,5-tetramethyl-2-(2-methylprop-1-en-1-yl)-1,3,2-dioxaborolane (0.385 g, 2.1 mmol), followed by K₂CO₃ (0.608 g, 4.4 mmol) and the resultant mixture was bubbled with argon for 20 min. Then Pd(dppf)Cl₂ (0.065 g, 0.088 mmol) was added after which the reaction was heated to 80° C. for 12 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure afforded 2-fluoro-4-(2-methylprop-1-en-1-yl)-6-(piperazin-1-yl)benzonitrile which was taken to the next step without additional purification.

### Step (iii): tert-Butyl 4-(2-cyano-3-fluoro-5-(2-methylprop-1-en-1-yl)phenyl)piperazine-1-carboxylate

To an ice-cold solution of 2-fluoro-4-(2-methylprop-1-en-1-yl)-6-(piperazin-1-yl)benzonitrile in CH₂Cl₂ (25 mL) was added Et₃N (0.62 mL, 4.4 mmol). The solution was stirred at 0° C. for 10 minutes, after which Boc-anhydride (0.575 g, 2.64 mmol) was added and the reaction was allowed to continue at room temperature for an additional 14 hours. After completion of the reaction was confirmed by TLC, cold water was added to the reaction mixture which was extracted with CH₂Cl₂ (3×300 mL). The combined organic layers were washed with sat. brine, dried over anhydrous sodium sulfate and evaporated under reduced pressure. The crude obtained was purified by column chromatography over silica gel (20-30% EtOAc in hexane) to afford tert-butyl 4-(2-cyano-3-fluoro-5-(2-methylprop-1-en-1-yl)phenyl)piperazine-1-carboxylate as a colorless gum (0.41 g, 65% over 2 steps).

### Step (iv): tert-Butyl 4-(2-cyano-3-fluoro-5-isobutylphenyl)piperazine-1-carboxylate

A stirred solution of tert-butyl 4-(2-cyano-3-fluoro-5-(2-methylprop-1-en-1-yl)phenyl)piperazine-1-carboxylate (0.4 g, 1.11 mmol) in MeOH (20 mL) was hydrogenated over 10% Pd/C (0.1 g) under 5 Kg/cm² H₂ pressure using a Parr hydrogenator for 2 h at ambient temperature. After confirming the completion of reaction by LC-MS, the reaction mixture was filtered through a Celite® bed and was concentrated in vacuo, yielding the desired compound as a colorless gum (0.36 g, 90%).

### Step (v): 2-Fluoro-4-isobutyl-6-(piperazin-1-yl)benzonitrile hydrochloride

tert-Butyl 4-(2-cyano-3-fluoro-5-isobutylphenyl)piperazine-1-carboxylate (0.35 g, 0.97 mmol) was dissolved in 4M HCl in 1,4-dioxane (20 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford 2-fluoro-4-isobutyl-6-(piperazin-1-yl)benzonitrile hydrochloride as an off-white solid (0.25 g, 89%).

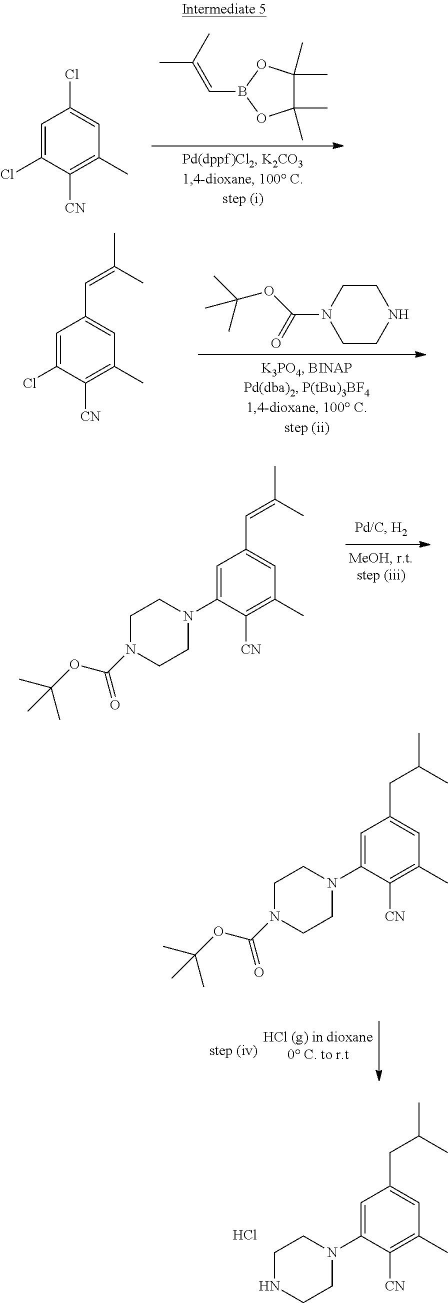

### Step (i): 2-Chloro-6-methyl-4-(2-methylprop-1-en-1-yl)benzonitrile

To a stirred solution of 2,4-dichloro-6-methylbenzonitrile (0.25 g, 1.34 mmol) in 1,4-dioxane (10 mL) was added 4,4,5,5-tetramethyl-2-(2-methylprop-1-en-1-yl)-1,3,2-dioxaborolane (0.27 g, 1.47 mmol), followed by K₂CO₃ (0.46 g, 3.35 mmol) and the resultant mixture was bubbled with argon for 20 min. Then Pd(dppf)Cl₂ (0.049 g, 0.067 mmol) was added after which the reaction was heated to 100° C. for 12 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (10-15% EtOAc in hexane) afforded 2-chloro-6-methyl-4-(2-methylprop-1-en-1-yl)benzonitrile as an off-white solid (0.21 g, 77%).

### Step (ii): tert-Butyl 4-(2-cyano-3-methyl-5-(2-methylprop-1-en-1-yl)phenyl)piperazine-1-carboxylate

To a stirred solution of 2-chloro-6-methyl-4-(2-methylprop-1-en-1-yl)benzonitrile (0.21 g, 1.02 mmol) in 1,4-dioxane (10 mL) was added tert-butyl piperazine-1-carboxylate (0.228 g, 1.23 mmol), followed by K₃PO₄ (0.541 g, 2.55 mmol) and BINAP (0.025 g, 0.041 mmol). The resultant mixture was bubbled with argon for 20 min. Then P(tBu)₃·BF₄ (0.012 g, 0.041 mmol) followed by Pd(dba)₂ (0.030 g, 0.051 mmol) were added after which the reaction was heated to 100° C. for 14 h. After the completion of reaction was confirmed by TLC, the reaction mixture was evaporated in vacuo to remove the volatiles and the residue was re-dissolved with ethyl acetate and washed with water and sat. brine. Drying over anhydrous sodium sulfate and concentration in vacuo, followed by column chromatography over silica gel (25-30% EtOAc in hexane) afforded the desired compound as a pale-yellow gum (0.15 g, 41%).

### Step (iii): tert-Butyl 4-(2-cyano-5-isobutyl-3-methylphenyl)piperazine-1-carboxylate

A stirred solution of tert-butyl 4-(2-cyano-3-methyl-5-(2-methylprop-1-en-1-yl)phenyl)piperazine-1-carboxylate (0.15 g, 0.42 mmol) in MeOH (20 mL) was hydrogenated over 10% Pd/C (40 mg) under 5 Kg/cm² H₂ pressure using a Parr hydrogenator for 2 h at ambient temperature. After confirming the completion of reaction by LC-MS, the reaction mixture was filtered through a Celite® bed and was concentrated in vacuo, yielding the envisaged hydrogenated compound as a colorless gum (0.12 g, 80%).

### Step (iv): 4-Isobutyl-2-methyl-6-(piperazin-1-yl)benzonitrile hydrochloride

tert-Butyl 4-(2-cyano-5-isobutyl-3-methylphenyl)piperazine-1-carboxylate (0.12 g, 0.336 mmol) was dissolved in 4M HCl in 1,4-dioxane (10 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford 4-isobutyl-2-methyl-6-(piperazin-1-yl)benzonitrile hydrochloride as an off-white solid (0.08 g, 81%).

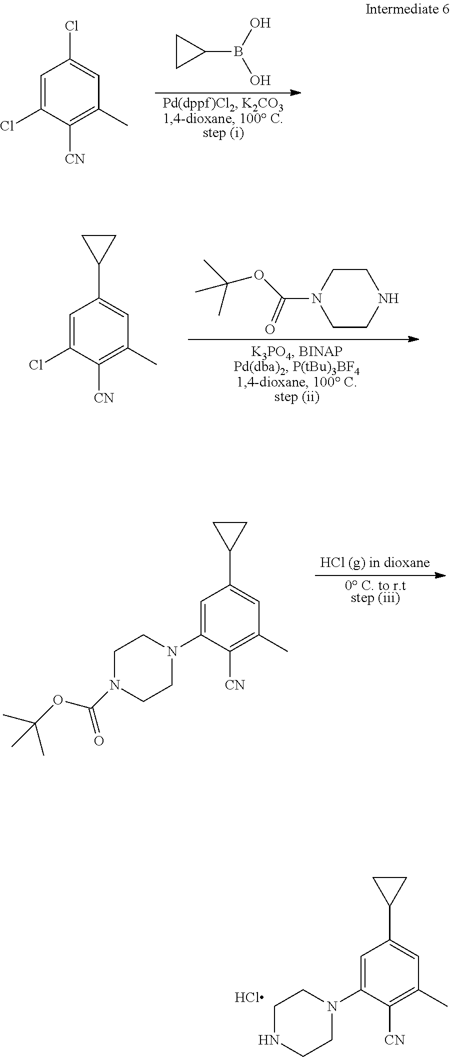

### Step (i): 2-Chloro-4-cyclopropyl-6-methylbenzonitrile

To a stirred solution of 2,4-dichloro-6-methylbenzonitrile (0.3 g, 1.61 mmol) in 1,4-dioxane (10 mL) was added cyclopropylboronic acid (0.152 g, 1.77 mmol), followed by K₂CO₃ (0.555 g, 4.025 mmol) and the resultant mixture was bubbled with argon for 20 min.

Then Pd(dppf)Cl₂ (0.058 g, 0.0805 mmol) was added after which the reaction was heated to 100° C. for 12 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (10-13% EtOAc in hexane) afforded 2-chloro-4-cyclopropyl-6-methylbenzonitrile as a gummy liquid (0.21 g, 68%).

### Step (ii): tert-Butyl 4-(2-cyano-5-cyclopropyl-3-methylphenyl)piperazine-1-carboxylate

To a stirred solution of 2-chloro-4-cyclopropyl-6-methylbenzonitrile (0.2 g, 1.047 mmol) in 1,4-dioxane (10 mL) was added tert-butyl piperazine-1-carboxylate (0.232 g, 1.25 mmol), followed by K₃PO₄ (0.555 g, 2.617 mmol) and BINAP (0.026 g, 0.0418 mmol). The resultant mixture was bubbled with argon for 20 min. Then P(tBu)₃·BF₄ (0.012 g, 0.0418 mmol) followed by Pd(dba)₂ (0.030 g, 0.0523 mmol) were added after which the reaction was heated to 100° C. for 14 h. After the completion of reaction was confirmed by TLC, the reaction mixture was evaporated in vacuo to remove the volatiles and the residue was re-dissolved with ethyl acetate and washed with water and sat. brine. Drying over anhydrous sodium sulfate and concentration in vacuo, followed by column chromatography over silica gel (25-30% EtOAc in hexane) afforded the desired compound as a pale-yellow gum (0.18 g, 51%).

### Step (iii): 4-Cyclopropyl-2-methyl-6-(piperazin-1-yl)benzonitrile hydrochloride

tert-Butyl 4-(2-cyano-5-cyclopropyl-3-methylphenyl)piperazine-1-carboxylate (0.18 g, 0.527 mmol) was dissolved in 4M HCl in 1,4-dioxane (10 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford 4-cyclopropyl-2-methyl-6-(piperazin-1-yl)benzonitrile hydrochloride as an off-white solid (0.12 g, 82%).

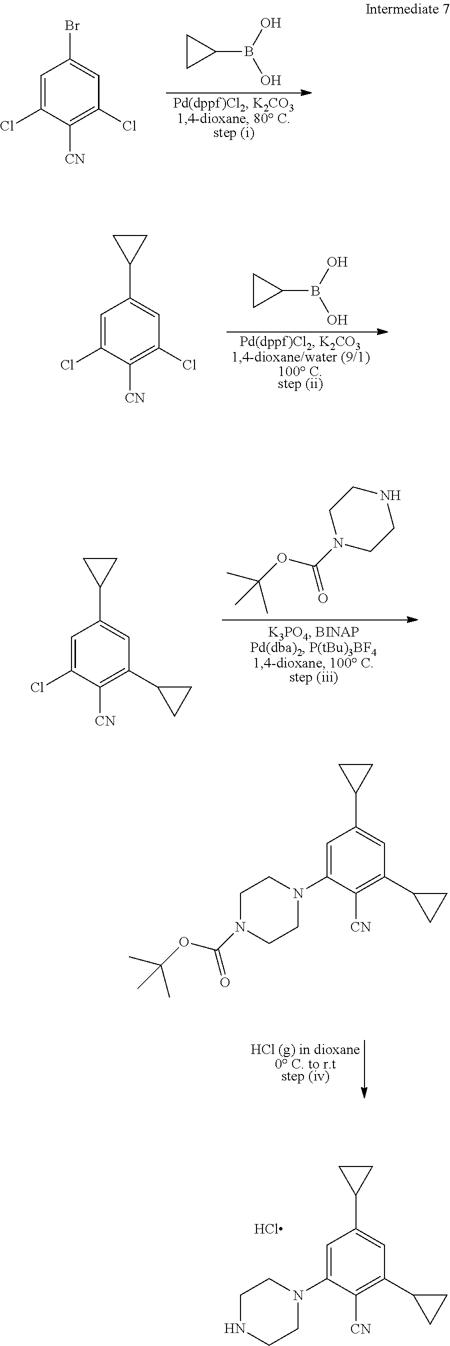

### Step (i): 2,6-Dichloro-4-cyclopropylbenzonitrile

To a stirred solution of 4-bromo-2,6-dichlorobenzonitrile (1 g, 3.985 mmol) in 1,4-dioxane (25 mL) was added cyclopropylboronic acid (0.411 g, 4.78 mmol), followed by K₂CO₃ (1.354 g, 9.963 mmol) and the resultant mixture was bubbled with argon for 20 min. Then Pd(dppf)Cl₂ (0.146 g, 0.1992 mmol) was added after which the reaction was heated to 80° C. for 12 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (10-12% EtOAc in hexane) afforded 2,6-dichloro-4-cyclopropylbenzonitrile as a gummy liquid (0.74 g, 88%).

### Step (ii): 2-Chloro-4,6-dicyclopropylbenzonitrile

To a stirred solution of 2,6-dichloro-4-cyclopropylbenzonitrile (0.37 g, 1.745 mmol) in 1,4-dioxane (20 mL) was added cyclopropylboronic acid (0.18 g, 2.06 mmol), followed by K₂CO₃ (0.593 g, 4.36 mmol) and the resultant mixture was bubbled with argon for 20 min. Then Pd(dppf)Cl₂ (0.064 g, 0.0873 mmol) was added after which the reaction was heated to 100° C. for 12 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (15-18% EtOAc in hexane) afforded 2-chloro-4,6-dicyclopropylbenzonitrile as a gummy liquid (0.3 g, 79%).

### Step (iii): tert-Butyl 4-(2-cyano-3,5-dicyclopropylphenyl)piperazine-1-carboxylate

To a stirred solution of 2-chloro-4,6-dicyclopropylbenzonitrile (0.3 g, 1.38 mmol) in 1,4-dioxane (20 mL) was added tert-butyl piperazine-1-carboxylate (0.308 g, 1.66 mmol), followed by K₃PO₄ (0.731 g, 3.45 mmol) and BINAP (0.034 g, 0.0552 mmol). The resultant mixture was bubbled with argon for 20 min. Then P(tBu)₃·BF₄ (0.016 g, 0.0552 mmol) followed by Pd(dba)₂ (0.040 g, 0.069 mmol) were added after which the reaction was heated to 100° C. for 14 h. After the completion of reaction was confirmed by TLC, the reaction mixture was evaporated in vacuo to remove the volatiles and the residue was re-dissolved with ethyl acetate and washed with water and sat. brine. Drying over anhydrous sodium sulfate and concentration in vacuo, followed by column chromatography over silica gel (30-35% EtOAc in hexane) afforded the desired compound as a pale-yellow gum (0.31 g, 60%).

### Step (iv): 2,4-Dicyclopropyl-6-(piperazin-1-yl)benzonitrile hydrochloride

tert-Butyl 4-(2-cyano-3,5-dicyclopropylphenyl)piperazine-1-carboxylate (0.3 g, 0.817 mmol) was dissolved in 4M HCl in 1,4-dioxane (10 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford 2,4-dicyclopropyl-6-(piperazin-1-yl)benzonitrile hydrochloride as an off-white solid (0.21 g, 82%).

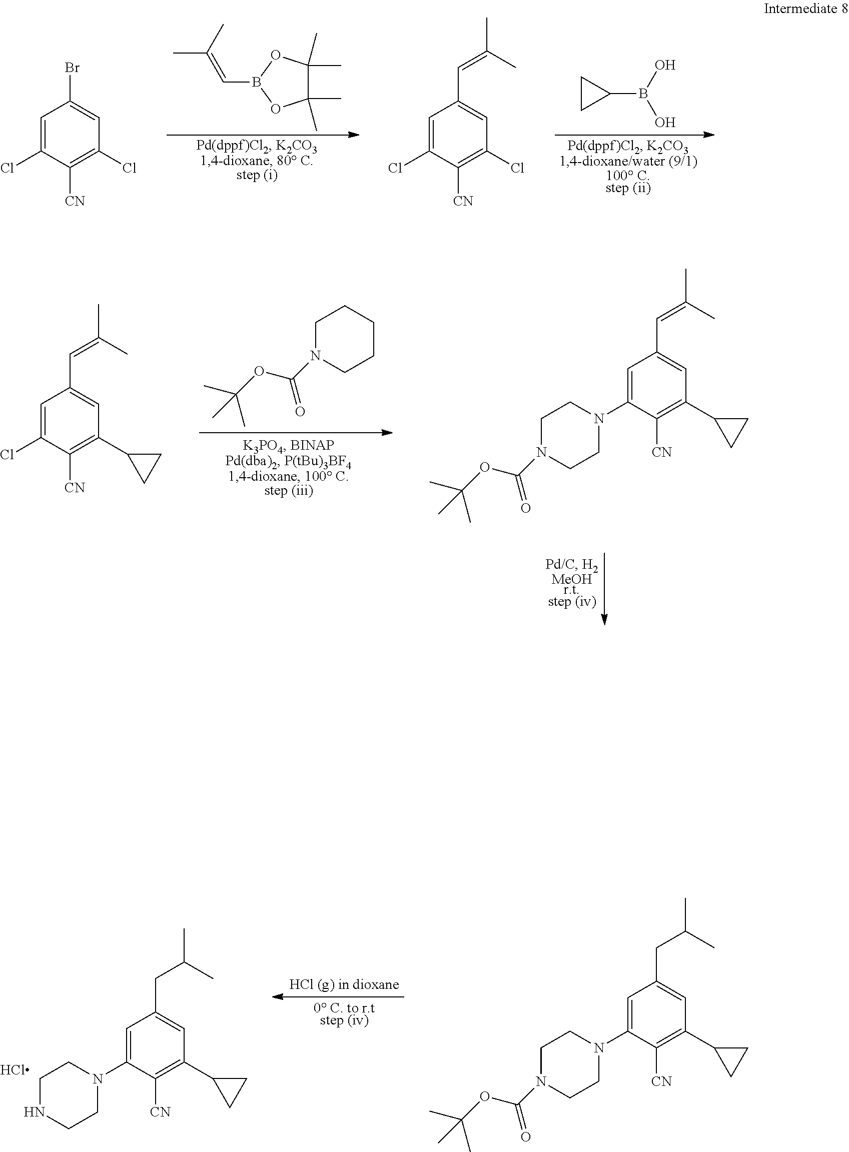

### Step (i): 2,6-Dichloro-4-(2-methylprop-1-en-1-yl)benzonitrile

To a stirred solution of 4-bromo-2,6-dichlorobenzonitrile (0.5 g, 2.0 mmol) in 1,4-dioxane (20 mL) was added 4,4,5,5-tetramethyl-2-(2-methylprop-1-en-1-yl)-1,3,2-dioxaborolane (0.436 g, 2.4 mmol), followed by K₂CO₃ (0.69 g, 5 mmol) and the resultant mixture was bubbled with argon for 20 min. Then Pd(dppf)Cl₂ (0.074 g, 0.10 mmol) was added after which the reaction was heated to 80° C. for 12 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (10-12% EtOAc in hexane) afforded 2,6-dichloro-4-(2-methylprop-1-en-1-yl)benzonitrile as a gummy liquid (0.4 g, 88%).

### Step (ii): 2-Chloro-6-cyclopropyl-4-(2-methylprop-1-en-1-yl)benzonitrile

To a stirred solution of 2,6-dichloro-4-(2-methylprop-1-en-1-yl)benzonitrile (0.2 g, 0.885 mmol) in 1,4-dioxane (10 mL) was added cyclopropylboronic acid (0.091 g, 1.06 mmol), followed by K₂CO₃ (0.305 g, 2.21 mmol) and the resultant mixture was bubbled with argon for 20 min. Then Pd(dppf)Cl₂ (0.033 g, 0.044 mmol) was added after which the reaction was heated to 100° C. for 12 h. After the completion of reaction was confirmed by LC-MS, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (10-15% EtOAc in hexane) afforded 2-chloro-6-cyclopropyl-4-(2-methylprop-1-en-1-yl)benzonitrile as a gummy liquid (0.175 g, 87%).

### Step (iii): tert-Butyl 4-(2-cyano-3-cyclopropyl-5-(2-methylprop-1-en-1-yl)phenyl)piperazine-1-carboxylate

To a stirred solution of 2-chloro-6-cyclopropyl-4-(2-methylprop-1-en-1-yl)benzonitrile (0.17 g, 0.735 mmol) in 1,4-dioxane (10 mL) was added tert-butyl piperazine-1-carboxylate (0.164 g, 0.883 mmol), followed by K₃PO₄ (0.390 g, 1.837 mmol) and BINAP (0.018 g, 0.029 mmol). The resultant mixture was bubbled with argon for 20 min. Then P(tBu)₃·BF₄ (0.009 g, 0.029 mmol) followed by Pd(dba)₂ (0.021 g, 0.368 mmol) were added after which the reaction was heated to 100° C. for 14 h. After the completion of reaction was confirmed by LC-MS, the reaction mixture was evaporated in vacuo to remove the volatiles and the residue was re-dissolved with ethyl acetate and washed with water and sat. brine. Drying over anhydrous sodium sulfate and concentration in vacuo, followed by column chromatography over silica gel (20-25% EtOAc in hexane) afforded the desired compound as a pale-yellow gum (0.16 g, 57%).

### Step (iv): tert-Butyl 4-(2-cyano-5-isobutyl-3-methylphenyl)piperazine-1-carboxylate

A stirred solution of tert-butyl 4-(2-cyano-3-cyclopropyl-5-(2-methylprop-1-en-1-yl)phenyl)piperazine-1-carboxylate (0.13 g, 0.341 mmol) in MeOH (20 mL) was hydrogenated over 10% Pd/C (30 mg) under 5 Kg/cm² H₂ pressure using a Parr hydrogenator for 2 h at ambient temperature. After confirming the completion of reaction by LC-MS, the reaction mixture was filtered through a Celite® bed and was concentrated in vacuo, yielding the envisaged hydrogenated compound as a colorless gum (0.11 g, 84%).

### Step (v): 2-Cyclopropyl-4-isobutyl-6-(piperazin-1-yl)benzonitrile hydrochloride

tert-Butyl 4-(2-cyano-5-isobutyl-3-methylphenyl)piperazine-1-carboxylate (0.10 g, 0.26 mmol) was dissolved in 4M HCl in 1,4-dioxane (5 mL) at 0° C., after which the solution was slowly warmed up to room temperature and continued stirring at r.t. for 2 h.

After the completion of the reaction was confirmed by TLC, the reaction mixture was evaporated to dryness under reduced pressure. The crude residue was further triturated with diethyl ether to afford 2-cyclopropyl-4-isobutyl-6-(piperazin-1-yl)benzonitrile hydrochloride as an off-white solid (0.06 g, 72%).

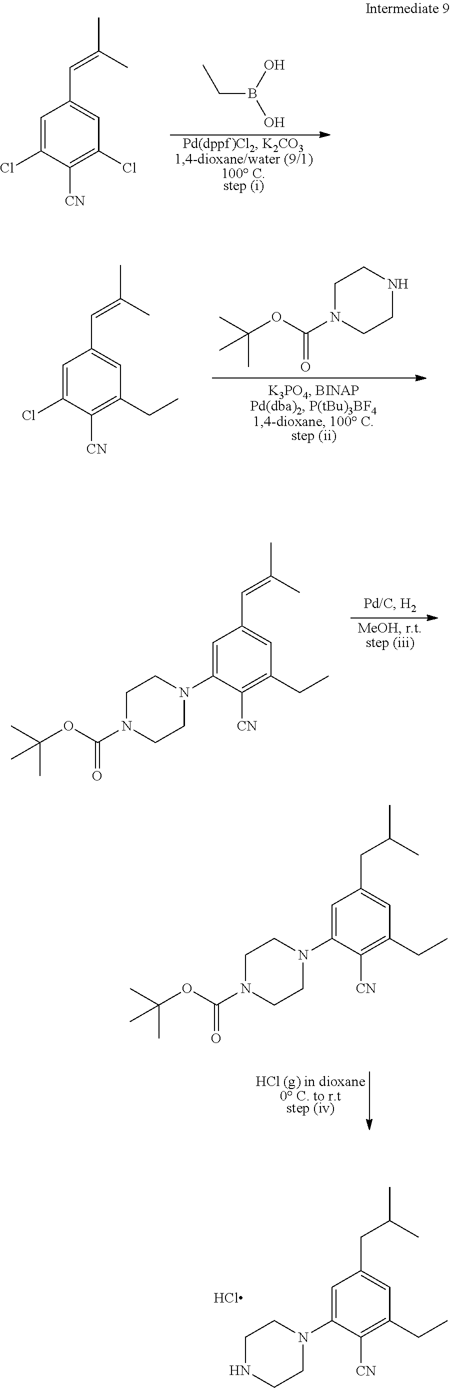

### Step (i): 2-Chloro-6-ethyl-4-(2-methylprop-1-en-1-yl)benzonitrile

To a stirred solution of 2,6-dichloro-4-(2-methylprop-1-en-1-yl)benzonitrile (0.2 g, 0.885 mmol) in 1,4-dioxane (10 mL) was added ethylboronic acid (0.078 g, 1.06 mmol), followed by K₂CO₃ (0.305 g, 2.21 mmol) and the resultant mixture was bubbled with argon for 20 min. Then Pd(dppf)Cl₂ (0.033 g, 0.044 mmol) was added after which the reaction was heated to 100° C. for 12 h. After the completion of reaction was confirmed by LC-MS, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (10-15% EtOAc in hexane) afforded 2-chloro-6-ethyl-4-(2-methylprop-1-en-1-yl)benzonitrile as a gummy liquid (0.169 g, 87%).

### Step (ii): tert-Butyl 4-(2-cyano-3-ethyl-5-(2-methylprop-1-en-1-yl)phenyl)piperazine-1-carboxylate

To a stirred solution of 2-chloro-6-ethyl-4-(2-methylprop-1-en-1-yl)benzonitrile (0.17 g, 0.735 mmol) in 1,4-dioxane (10 mL) was added tert-butyl piperazine-1-carboxylate (0.164 g, 0.883 mmol), followed by K₃PO₄ (0.390 g, 1.837 mmol) and BINAP (0.018 g, 0.029 mmol). The resultant mixture was bubbled with argon for 20 min. Then P(tBu)₃·BF₄ (0.009 g, 0.029 mmol) followed by Pd(dba)₂ (0.021 g, 0.368 mmol) were added after which the reaction was heated to 100° C. for 14 h. After the completion of reaction was confirmed by LC-MS, the reaction mixture was evaporated in vacuo to remove the volatiles and the residue was re-dissolved with ethyl acetate and washed with water and sat. brine. Drying over anhydrous sodium sulfate and concentration in vacuo, followed by column chromatography over silica gel (20-25% EtOAc in hexane) afforded the desired compound as a pale-yellow gum (0.155 g, 57%).

### Step (iii): tert-Butyl 4-(2-cyano-3-ethyl-5-isobutylphenyl)piperazine-1-carboxylate

A stirred solution of tert-butyl 4-(2-cyano-3-ethyl-5-(2-methylprop-1-en-1-yl)phenyl)piperazine-1-carboxylate (0.155 g, 0.419 mmol) in MeOH (20 mL) was hydrogenated over 10% Pd/C (40 mg) under 5 Kg/cm² H₂ pressure using a Parr hydrogenator for 2 h at ambient temperature. After confirming the completion of reaction by LC-MS, the reaction mixture was filtered through a Celite® bed and was concentrated in vacuo, yielding the envisaged hydrogenated compound as a colorless gum (0.145 g, 93%).

### Step (iv): 2-Ethyl-4-isobutyl-6-(piperazin-1-yl)benzonitrile hydrochloride

tert-Butyl 4-(2-cyano-3-ethyl-5-isobutylphenyl)piperazine-1-carboxylate (0.145 g, 0.390 mmol) was dissolved in 4M HCl in 1,4-dioxane (5 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford 2-ethyl-4-isobutyl-6-(piperazin-1-yl)benzonitrile hydrochloride as an off-white solid (0.097 g, 92%).

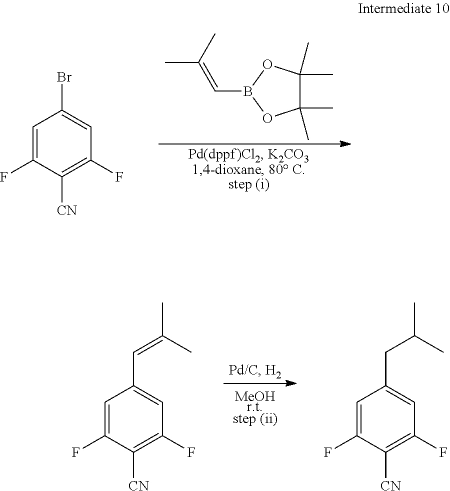

### Step (i): 2,6-Difluoro-4-(2-methylprop-1-en-1-yl)benzonitrile

To a stirred solution of 4-bromo-2,6-dichlorobenzonitrile (10 g, 45.87 mmol) in 1,4-dioxane was added 4,4,5,5-tetramethyl-2-(2-methylprop-1-en-1-yl)-1,3,2-dioxaborolane (10.01 g, 55.05 mmol), followed by K₂CO₃ (15.82 g, 114.68 mmol) and the resultant mixture was bubbled with argon for 20 min. Then Pd(dppf)Cl₂ (3.37 g, 4.58 mmol) was added after which the reaction was heated to 80° C. for 6 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (15-20% EtOAc in hexane) afforded 2,6-difluoro-4-(2-methylprop-1-en-1-yl)benzonitrile as a colorless gum (7.10 g, 80%).

### Step (ii): 2,6-Difluoro-4-isobutylbenzonitrile

A stirred solution of 2,6-difluoro-4-(2-methylprop-1-en-1-yl)benzonitrile (7.0 g, 36.2 mmol) in MeOH was hydrogenated over 10% Pd/C (1.4 g) under 5 Kg/cm² H₂ pressure using a Parr hydrogenator for 3 h at ambient temperature. After confirming the completion of reaction by LC-MS, the reaction mixture was filtered through a Celite® bed and was concentrated in vacuo, yielding the envisaged hydrogenated compound as a viscous liquid (5.81 g, 83%).

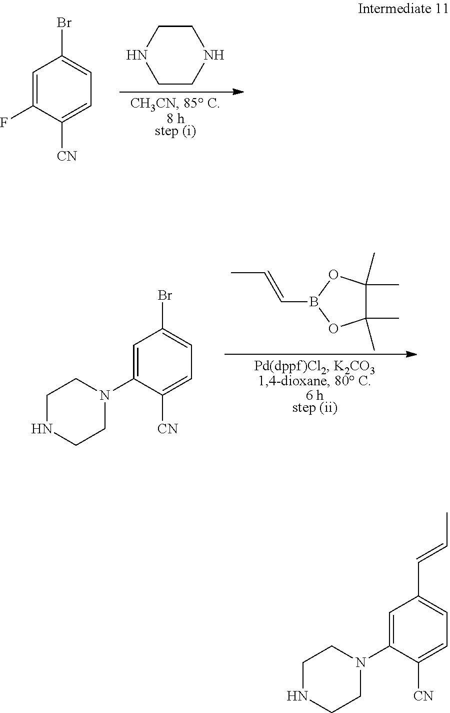

### Step (i): 4-Bromo-2-(piperazin-1-yl)benzonitrile

After addition of piperazine (431 mg, 5.0 mmol) to a stirred solution of 4-bromo-2-fluorobenzonitrile (200 mg, 1.0 mmol) in acetonitrile (5 mL), the reaction was continued at 85° C. for 8 h. After completion of the reaction was confirmed by TLC, the reaction mixture was evaporated to dryness, diluted with water and extracted with EtOAc. The combined organic layers were washed with sat. brine, dried over anhydrous sodium sulfate and evaporated under reduced pressure. The crude gummy colorless liquid was taken to the next step without purification.

### Step (ii): 2-(Piperazin-1-yl)-4-(prop-1-en-1-yl)benzonitrile

To a stirred solution of 4-bromo-2-(piperazin-1-yl)benzonitrile (0.25 g, 0.94 mmol) in 1,4-dioxane (20 mL) was added 4,4,5,5-tetramethyl-2-(prop-1-en-1-yl)-1,3,2-dioxaborolane (0.19 g, 1.1 mmol), followed by K₂CO₃ (0.39 g, 2.81 mmol) and the resultant mixture was bubbled with argon for 20 min. Then Pd(dppf)Cl₂ (0.068 g, 0.09 mmol) was added after which the reaction was heated to 80° C. for 6 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and solvent evaporation under reduced pressure afforded 2-(piperazin-1-yl)-4-(prop-1-en-1-yl)benzonitrile (47.0 g) which was taken to the next step without additional purification (used in method L, step 2).

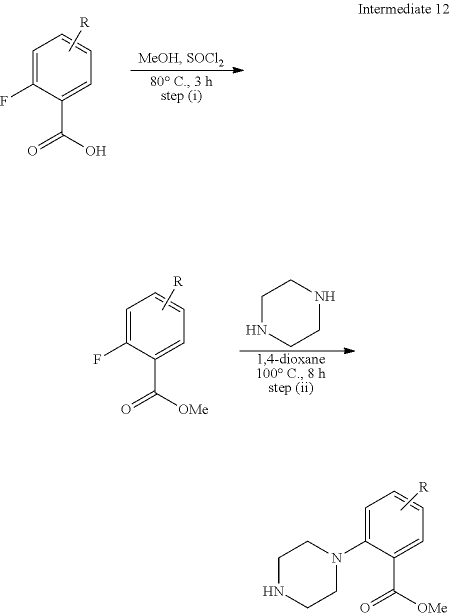

### Step (i): Substituted methyl 2-fluorobenzoate

To a stirred solution of a substituted 2-fluorobenzoic acid (3.5 mmol) in MeOH (25 mL) was added SOCl₂ (0.8 mL, 10.7 mmol) at 0° C., after which the reaction was brought to 80° C. and kept stirring at this temperature for 3 h. After the completion of the reaction was confirmed by TLC, the reaction mixture was evaporated to dryness, diluted with water, washed with sat. NaHCO₃ and extracted with ethyl acetate. The combined organic layers were washed with water, sat. brine and dried over anhydrous sodium sulfate. Evaporation under reduced pressure afforded a crude residue which was taken for the next step without additional purification.

### Step (ii): Substituted methyl 2-(piperazin-1-yl)benzoate

To a stirred solution of a substituted methyl 2-fluorobenzoate of interest (3.2 mmol) in 1,4-dioxane (50 mL) was added piperazine (1.4 g, 16.2 mmol). Upon completion of the addition, the reaction was kept stirring at 100° C. for 8 h. After the completion of the reaction was confirmed by TLC, the reaction mixture was evaporated to dryness, water was added and the resulting solution was extracted with EtOAc. The organic layer was washed with sat. brine, dried over anhydrous sodium sulfate and evaporated in vacuo to afford a crude residue, which after column chromatography (SiO₂, 3-10% MeOH in CH₂Cl₂), afforded the desired substituted methyl 2-(piperazin-1-yl)benzoate.

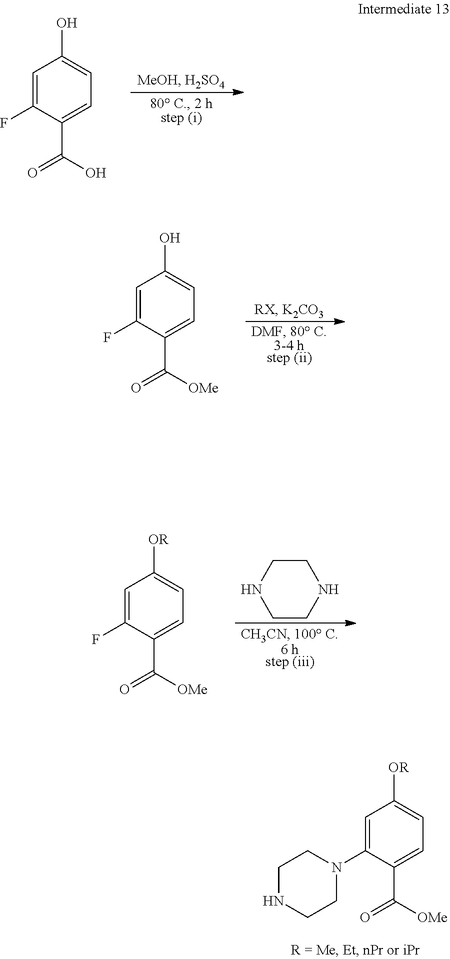

### Step (i): Methyl 2-fluoro-4-hydroxybenzoate

To a stirred solution of 2-fluoro-4-hydroxybenzoic acid (1.0 g, 6.4 mmol) in MeOH (30 mL) was added H₂SO₄ (10 mL, 64.0 mmol) at 0° C., after which the reaction was brought to 80° C. for 2 h. After the completion of the reaction was confirmed by TLC, the reaction mixture was evaporated to dryness, diluted with water, washed with sat. NaHCO₃ and extracted with ethyl acetate. The combined organic layers were washed with water, sat. brine and dried over anhydrous sodium sulfate. Evaporation under reduced pressure afforded a crude residue (1.08 g crude) which was taken for the next step without purification.

### Step (ii): Methyl 4-alkoxy 2-fluoro-benzoate

To a stirred solution of methyl 2-fluoro-4-hydroxybenzoate (1.0 g, 5.9 mmol) in DMF (25 mL) was added K₂CO₃ (1.62 g, 11.75 mmol) at 0° C., after which the solution was allowed to warm up to room temperature. After stirring at r.t. for 10 minutes, an alkyl halide of interest (8.81 mmol) was added, after which the reaction mixture was brought to 80° C. and kept stirring at this temperature for 3-4 h. After completion of the reaction was confirmed by TLC, water was added to the reaction mixture and extraction with ethyl acetate was performed. The combined organic layers were washed with water, sat. brine, dried over anhydrous sodium sulfate and evaporated under reduced pressure to afford a crude residue. The obtained residual compound was purified by column chromatography over silica gel, eluting with 10-15% EtOAc in hexane, affording the envisaged methyl 4-alkoxy 2-fluoro-benzoate.

### Step (iii): Methyl 4-alkoxy-2-(piperazin-1-yl)benzoate

To a stirred solution of methyl 4-alkoxy 2-fluoro-benzoate (4.71 mmol) in acetonitrile (30 mL) was added piperazine (23.5 mmol), after which the resulting solution was stirred at 100° C. for 6 h. After completion of the reaction was confirmed by TLC, the reaction mixture was evaporated to dryness, water was added, after which the obtained solution was extracted with EtOAc. The combined organic layers were washed with sat. brine, dried over anhydrous sodium sulfate and evaporated under reduced pressure to afford the methyl 4-alkoxy-2-(piperazin-1-yl)benzoate of interest. The crude compound was taken to the next step without any additional purification.

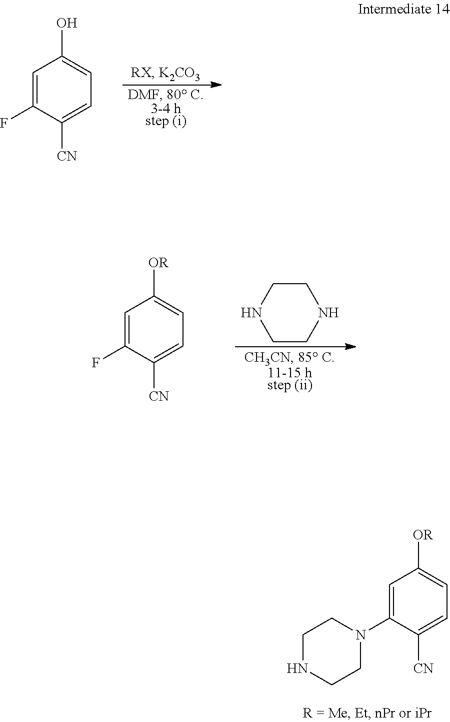

### Step (i): 4-Alkoxy-2-fluorobenzonitrile

To a stirred solution of 2-fluoro-4-hydroxybenzonitrile (1.0 mmol) in DMF was added K₂CO₃ (3.0 mmol) at 0° C., after which the solution was allowed to warm up to room temperature. After stirring at r.t. for 10 minutes, an alkyl halide of interest (1.2 mmol) was added, after which the reaction mixture was brought to 80° C. and kept stirring at this temperature for 3-4 h. After completion of the reaction was confirmed by TLC, the reaction mixture was extracted with water and ethyl acetate. The combined organic layers were washed with water, sat. brine, dried over anhydrous sodium sulfate and evaporated under reduced pressure to afford a crude residue. The obtained residual compound was purified by column chromatography over silica gel, eluting with EtOAc in hexane, affording the 4-alkoxy-2-fluorobenzonitrile.

### Step (ii): 4-Alkoxy-2-(piperazin-1-yl)benzonitrile

To a stirred solution of 4-alkoxy-2-fluorobenzonitrile (1 equiv.) in acetonitrile was added piperazine (5 equiv.), after which the resulting solution was stirred at 85° C. for 11-15 h. After completion of the reaction was confirmed by TLC, the reaction mixture was evaporated to dryness, water was added and the resulting solution was extracted with EtOAc. The combined organic layers were washed with sat. brine, dried over anhydrous sodium sulfate and evaporated under reduced pressure to afford 4-alkoxy-2-(piperazin-1-yl)benzonitrile as a colorless oil. The crude compound was taken to the next step without any additional purification (used in method L, step 2).

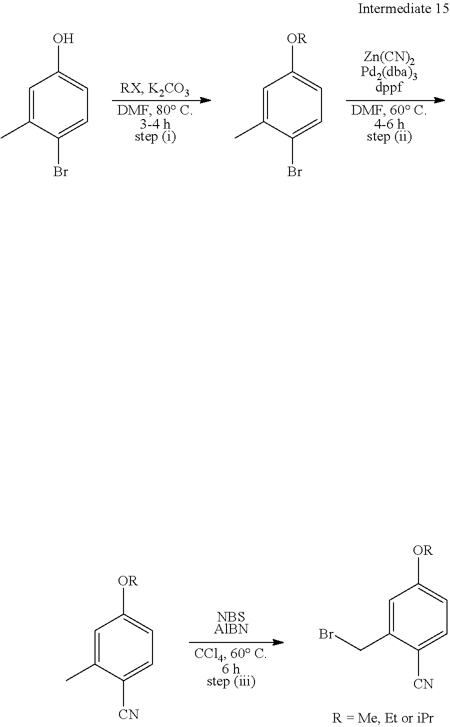

### Step (i): 4-Alkoxy-l-bromo-2-methylbenzene

To a stirred solution of 4-bromo-3-methylphenol (1.0 g, 5.3 mmol) in DMF (25 mL) was added K₂CO₃ (1.44 g, 10.7 mmol) at 0° C., after which the solution was allowed to warm up to room temperature. After stirring at r.t. for 10 minutes, an alkyl halide of interest (8.02 mmol) was added, after which the reaction mixture was brought to 80° C. and kept stirring at this temperature for 3-4 h. After completion of the reaction was confirmed by TLC, water was added to the reaction mixture which was extracted with ethyl acetate. The combined organic layers were washed with water, sat. brine, dried over anhydrous sodium sulfate and evaporated under reduced pressure to afford a crude residue. The obtained residual compound was purified by column chromatography over silica gel, eluting with EtOAc in hexane, affording the 4-alkoxy-1-bromo-2-methylbenzene.

### Step (ii): 4-Alkoxy-2-methylbenzonitrile

To a stirred solution of 4-alkoxy-1-bromo-2-methylbenzene (4.36 mmol) in DMF (30 mL) was added Zn(CN)₂ (0.77 g, 6.54 mmol), followed by a catalytic amount of dppf (0.024 g, 0.043 mmol). The resulting reaction mixture was bubbled with argon for 20 min, after which Pd₂(dba)₃ (0.039 g, 0.043 mmol) was added and the reaction brought to 60° C. After completion of reaction was confirmed by TLC, water was added to the reaction mixture which was extracted with ethyl acetate. The combined organic layers were washed with water, sat. brine, dried over anhydrous sodium sulfate and evaporated under reduced pressure. The obtained crude compound was purified by column chromatography over silica gel, eluting with EtOAc in hexane, to afford the 4-alkoxy-2-methylbenzonitrile of interest.

### Step (iii): 4-Alkoxy-2-(bromomethyl)benzonitrile

To a stirred solution of 4-alkoxy-2-methylbenzonitrile (1.99 mmol) in CCl₄ (25 mL) was added NBS (0.71 g, 3.99 mmol), followed by AIBN (0.032 g, 0.2 mmol). Upon completion of the addition, the resulting reaction mixture was heated to 60° C. for 6 h. After completion of reaction was confirmed by TLC, the reaction mixture was cooled down and cold water was added. Extraction with ethyl acetate was performed, after which the combined organic layers were subsequently washed with water and sat. brine. Drying over anhydrous sodium sulfate and solvent evaporation under reduced pressure, yielded a crude residue, which was further purified by column chromatography over silica gel (10-15% EtOAc in hexane).

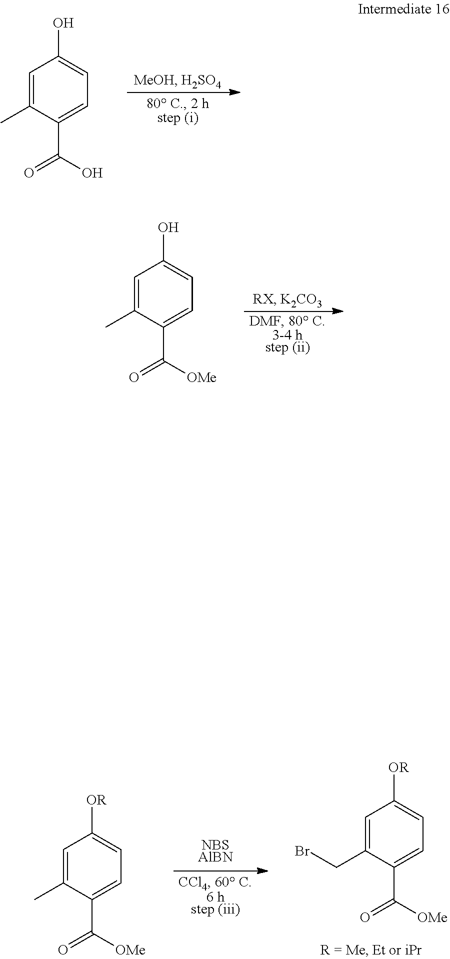

### Step (i): Methyl 4-hydroxy-2-methylbenzoate

To a stirred solution of 4-hydroxy-2-methylbenzoic acid (1.0 g, 6.57 mmol) in MeOH (30 mL) was added H₂SO₄ (10 mL) at 0° C., after which the reaction was brought to 80° C. for 2 h. After the completion of the reaction was confirmed by TLC, the reaction mixture was evaporated to dryness, water was added to the reaction mixture which was treated with sat. NaHCO₃ and extracted with ethyl acetate. The combined organic layers were washed with water, sat. brine and dried over anhydrous sodium sulfate. Evaporation under reduced pressure afforded a crude residue (1.12 g crude) which was taken for the next step without further purification.

### Step (ii): Methyl 4-alkoxy-2-methylbenzoate

To a stirred solution of methyl 4-hydroxy-2-methylbenzoate (1.0 g, 6.01 mmol) in DMF (30 mL) was added K₂CO₃ (1.66 g, 12.03 mmol) at 0° C., after which the solution was allowed to warm up to room temperature. After stirring at r.t. for 10 minutes, an alkyl halide of interest (9.02 mmol) was added, after which the reaction mixture was brought to 80° C. and kept stirring at this temperature for 3-4 h. After completion of the reaction was confirmed by TLC, water was added to the reaction mixture which was then extracted with ethyl acetate. The combined organic layers were washed with water, sat. brine, dried over anhydrous sodium sulfate and evaporated under reduced pressure to afford a crude residue. The obtained residual compound was purified by column chromatography over silica gel, eluting with EtOAc in hexane, affording methyl 4-alkoxy-2-methylbenzoate.

### Step (iii): Methyl 4-alkoxy-2-(bromomethyl)benzoate

To a stirred solution of methyl 4-alkoxy-2-methylbenzoate (2.40 mmol) in CCl₄ (30 mL) was added NBS (0.85 g, 4.80 mmol), followed by AIBN (0.035 g, 0.24 mmol). Upon completion of the addition, the resulting reaction mixture was heated to 60° C. for 6 h. After completion of reaction was confirmed by TLC, the reaction mixture was cooled down and cold water was added. Extraction with ethyl acetate was performed, after which the combined organic layers were subsequently washed with water and sat. brine. Drying over anhydrous sodium sulfate and solvent evaporation under reduced pressure, yielded a crude residue, which was further purified by column chromatography over silica gel (10-20% EtOAc in hexane).

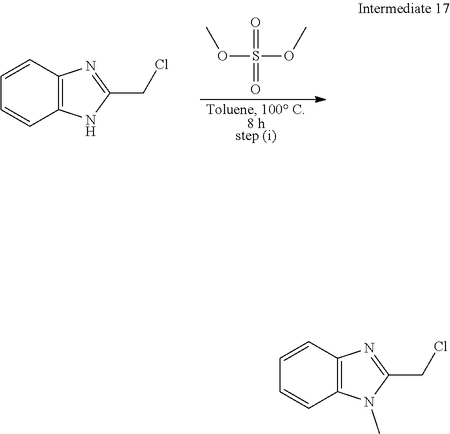

### Step (i): 2-(Chloromethyl)-1-methyl-1H-benzo[d]imidazole

To a stirred solution of 2-(chloromethyl)-1H-benzo[d]imidazole (5 g, 30.0 mmol) in anhydrous toluene (50 mL) was added dimethyl sulfate (4.26 mL, 45.0 mmol) after which the solution was stirred at 100° C. for 8 h. After the completion of the reaction was confirmed by TLC, the reaction mixture was evaporated to dryness, water was added to the solution which was then extracted with ethyl acetate (2×50 mL). The combined organic layers were washed with sat. brine, dried over anhydrous sodium sulfate and evaporated in vacuo to afford the envisaged methylated heterocycle as a pale-yellow solid (5.6 g, crude). The crude compound was used in the following steps without additional purification.

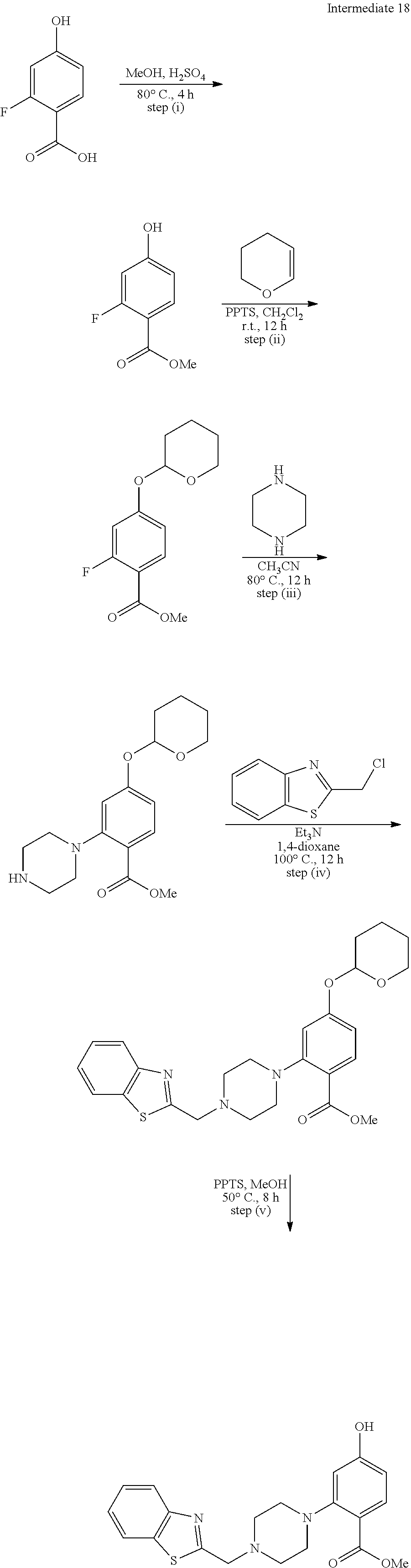

### Step (i): Methyl 2-fluoro-4-hydroxybenzoate

To a stirred solution of 2-fluoro-4-hydroxybenzoic acid (5.0 g, 32.03 mmol) in MeOH (50 mL) was added H₂SO₄ (25 mL) at 0° C., after which the reaction was brought to 80° C. for 4 h. After the completion of the reaction was confirmed by TLC, the reaction mixture was evaporated to dryness, water was added followed by treatment of sat. NaHCO₃ and extraction with ethyl acetate. The combined organic layers were washed with water, sat. brine and dried over anhydrous sodium sulfate. Evaporation under reduced pressure afforded a crude residue (5.11 g crude) which was used as such in the next step without further purification.

### Step (ii): Methyl 2-fluoro-4-((tetrahydro-2H-pyran-2-yl)oxy)benzoate

To a stirred solution of methyl 2-fluoro-4-hydroxybenzoate (5.0 g, 29.4 mmol) in anhydrous CH₂Cl₂ (75 mL) at room temperature was added PPTS (804 mg, 2.94 mmol). After stirring for 10 minutes, 3,4-dihydro-2H-pyran (4.94 g, 58.79 mmol) was added, after which the reaction mixture was kept stirring at r.t. for 12 h. After completion of the reaction was confirmed by TLC, the reaction mixture was diluted with water and extracted with CH₂Cl₂. The combined organic layers were washed with water, sat. brine, dried over anhydrous sodium sulfate and concentrated in vacuo to afford a crude residue. The obtained residual compound was purified by column chromatography over silica gel, eluting with 10-15% EtOAc in hexane, affording methyl 2-fluoro-4-((tetrahydro-2H-pyran-2-yl)oxy)benzoate (6.81 g, 91%).

### Step (iii): Methyl 2-(piperazin-1-yl)-4-((tetrahydro-2H-pyran-2-yl)oxy)benzoate

To a stirred solution of methyl 2-fluoro-4-((tetrahydro-2H-pyran-2-yl)oxy)benzoate (6.0 g, 23.60 mmol) in acetonitrile (100 mL) was added piperazine (10.15 g, 118.02 mmol), after which the resulting solution was stirred at 80° C. for 12 h. After completion of the reaction was confirmed by TLC, the reaction mixture was evaporated to dryness, water was added and the obtained solution was extracted with EtOAc. The combined organic layers were washed with sat. brine, dried over anhydrous sodium sulfate and evaporated under reduced pressure to afford a crude residue (6.8 g crude), which was taken to the next step without any additional purification.

### Step (iv): Methyl 2-(4-(benzo[d]thiazol-2-ylmethyl)piperazin-1-yl)-4-((tetrahydro-2H-pyran-2-yl)oxy)benzoate

To a stirred solution of methyl 2-(piperazin-1-yl)-4-((tetrahydro-2H-pyran-2-yl)oxy)benzoate (6.5 g, 20.29 mmol) in 1,4-dioxane (60 mL) was added Et₃N (8.43 mL, 60.86 mmol) at 0° C., followed after 10 minutes by the addition of 2-(chloromethyl)benzo[d]thiazole (5.54 g, 30.43 mmol). The resulting reaction mixture was stirred at 100° C. for 12 h. After completion of the reaction was confirmed by TLC, water was added to the reaction mixture and the obtained mixture was extracted with ethyl acetate. The combined organic layers were washed with water, sat. brine, dried over anhydrous sodium sulfate and evaporated in vacuo to afford a crude residue. The crude compound was further purified by column chromatography over silica gel (30-40% EtOAc in hexane), yielding the desired compound (6.3 g, 78%).

### Step (v): Methyl 2-(4-(benzo[d]thiazol-2-ylmethyl)piperazin-1-yl)-4-hydroxybenzoate

To a stirred solution of methyl 2-(4-(benzo[d]thiazol-2-ylmethyl)piperazin-1-yl)-4-((tetrahydro-2H-pyran-2-yl)oxy)benzoate (6.0 g, 12.83 mmol) in MeOH (50 mL) was added PPTS (322 mg, 1.28 mmol), after which the resulting solution was stirred at 50° C. for 8 h. After completion of the reaction was confirmed by TLC, the reaction mixture was evaporated to dryness, water was added and the obtained mixture was extracted with EtOAc. The combined organic layers were washed with sat. brine, dried over anhydrous sodium sulfate and concentrated to afford a crude residue, which was purified via column chromatography (SiO₂, 40-50% EtOAc in hexane), affording the envisaged product (3.42 g, 70%).

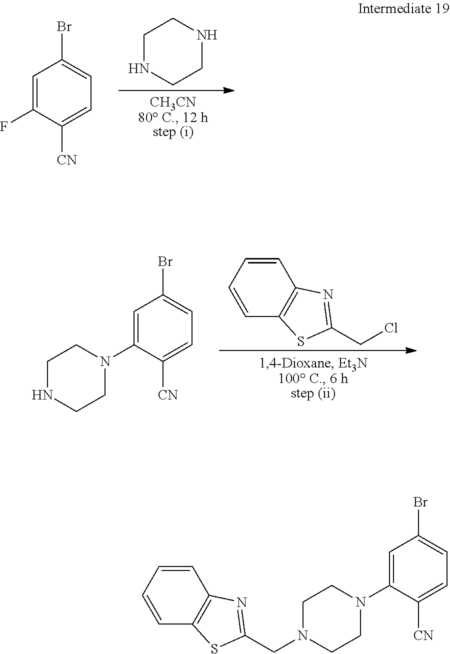

### Step (i): 4-Bromo-2-(piperazin-1-yl)benzonitrile

To a stirred solution of 4-bromo-2-fluorobenzonitrile (10.0 g, 50.0 mmol) in acetonitrile (100 mL) was added piperazine (21.5 g, 250.0 mmol), after which the resulting solution was stirred at 80° C. for 12 h. After completion of the reaction was confirmed by TLC, the reaction mixture was evaporated to dryness, water was added and the obtained mixture was extracted with EtOAc (3×200 mL). The combined organic layers were washed with sat. brine, dried over anhydrous sodium sulfate and evaporated in vacuo to afford 4-bromo-2-(piperazin-1-yl)benzonitrile (12.1 g crude) which was taken as such to the next step.

### Step (iii): 2-(4-(Benzo[d]thiazol-2-ylmethyl)piperazin-1-yl)-4-bromobenzonitrile

To a stirred solution of 4-bromo-2-(piperazin-1-yl)benzonitrile (6.0 g, 22.5 mmol) in 1,4-dioxane (50 mL) was added Et₃N (9.37 mL, 67.6 mmol) at 0° C., followed after 10 minutes by the addition of 2-(chloromethyl)benzo[d]thiazole (4.98 g, 27.06 mmol). The resulting reaction mixture was stirred at 100° C. for 6 h. After completion of the reaction was confirmed by TLC, cold water was added to the reaction mixture which was then extracted with CH₂Cl₂ (3×20 mL). The combined organic layers were washed with sat. brine, dried over anhydrous sodium sulfate and concentrated under reduced pressure to afford a crude residue, which was further purified by column chromatography over silica gel (30-40% EtOAc in hexane), yielding the envisaged compound 2-(4-(benzo[d]thiazol-2-ylmethyl)piperazin-1-yl)-4-bromobenzonitrile as a pale-yellow solid (5.3 g, 57%).

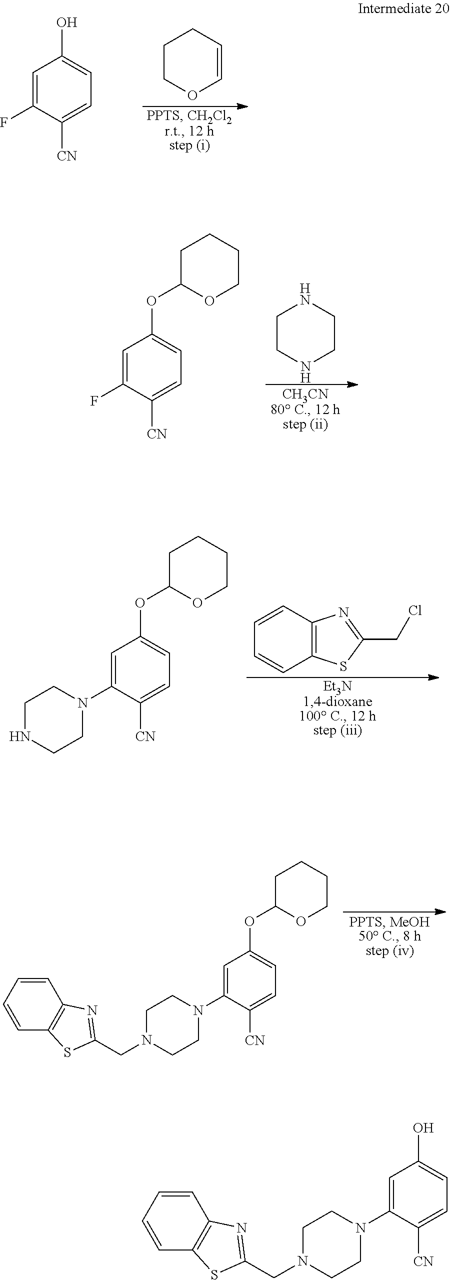

### Step (i): 2-Fluoro-4-((tetrahydro-2H-pyran-2-yl)oxy)benzonitrile

To a stirred solution of 2-fluoro-4-hydroxybenzonitrile (7.5 g, 54.7 mmol) in anhydrous CH₂Cl₂ (75 mL) at room temperature was added PPTS (1.37 g, 5.47 mmol). After stirring for 10 minutes, 3,4-dihydro-2H-pyran (9.2 g, 109.4 mmol) was added, after which the reaction mixture was kept stirring at r.t. for 12 h. After completion of the reaction was confirmed by TLC, water was added to the reaction mixture which was then extracted with CH₂Cl₂. The combined organic layers were washed with water, sat. brine, dried over anhydrous sodium sulfate and concentrated in vacuo to afford a crude residue. The obtained residual compound was purified by column chromatography over silica gel, eluting with 10-15% EtOAc in hexane, affording 2-fluoro-4-((tetrahydro-2H-pyran-2-yl)oxy)benzonitrile (11.0 g, 91%).

### Step (ii): 2-(Piperazin-1-yl)-4-((tetrahydro-2H-pyran-2-yl)oxy)benzonitrile

To a stirred solution of 2-fluoro-4-((tetrahydro-2H-pyran-2-yl)oxy)benzonitrile (8.0 g, 36.17 mmol) in acetonitrile (150 mL) was added piperazine (15.6 g, 180.8 mmol), after which the resulting solution was stirred at 80° C. for 12 h. After completion of the reaction was confirmed by TLC, the reaction mixture was evaporated to dryness, water was added and the obtained solution was extracted with EtOAc. The combined organic layers were washed with sat. brine, dried over anhydrous sodium sulfate and evaporated under reduced pressure to afford a crude residue (8.5 g crude), which was taken to the next step without any additional purification.

### Step (iii): 2-(4-(Benzo[d]thiazol-2-ylmethyl)piperazin-1-yl)-4-((tetrahydro-2H-pyran-2-yl)oxy)benzonitrile

To a stirred solution of 2-(piperazin-1-yl)-4-((tetrahydro-2H-pyran-2-yl)oxy)benzonitrile (8.0 g, 27.85 mmol) in 1,4-dioxane (100 mL) was added Et₃N (11.6 mL, 83.5 mmol) at 0° C., followed after 10 minutes by the addition of 2-(chloromethyl)benzo[d]thiazole (6.08 g, 33.4 mmol). The resulting reaction mixture was stirred at 100° C. for 12 h. After completion of the reaction was confirmed by TLC, water was added to the reaction mixture which was then extracted with ethyl acetate. The combined organic layers were washed with water, sat. brine, dried over anhydrous sodium sulfate and evaporated in vacuo to afford a crude residue. The crude compound was further purified by column chromatography over silica gel (30-40% EtOAc in hexane), yielding the desired compound (8.2 g, 68%).

### Step (iv): 2-(4-(Benzo[d]thiazol-2-ylmethyl)piperazin-1-yl)-4-hydroxybenzonitrile

To a stirred solution of 2-(4-(benzo[d]thiazol-2-ylmethyl)piperazin-1-yl)-4-((tetrahydro-2H-pyran-2-yl)oxy)benzonitrile (8.0 g, 18.41 mmol) in MeOH (100 mL) was added PPTS (462 mg, 1.84 mmol), after which the resulting solution was stirred at 50° C. for 8 h. After completion of the reaction was confirmed by TLC, the reaction mixture was evaporated to dryness, water was added and the obtained solution extracted with EtOAc. The combined organic layers were washed with sat. brine, dried over anhydrous sodium sulfate and concentrated to afford a crude residue, which was purified via column chromatography (SiO₂, 40-50% EtOAc in hexane), affording the envisaged product (3.2 g, 50%).

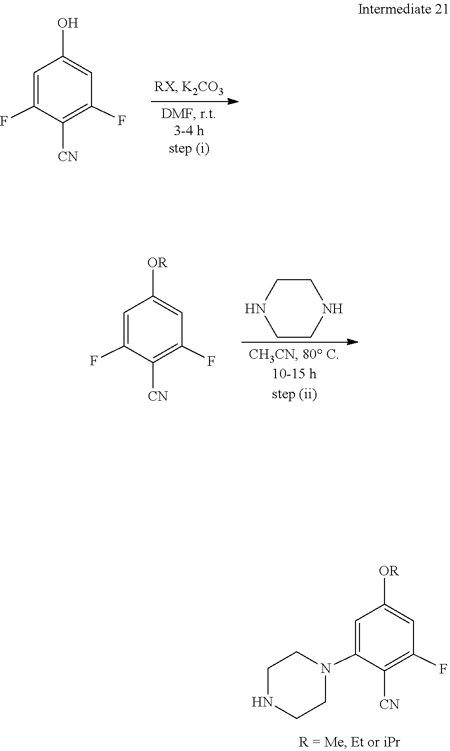

### Step (i): 4-Alkoxy-2,6-difluorobenzonitrile

To a stirred solution of 2,6-difluoro-4-hydroxybenzonitrile (1 equiv.) in DMF was added K₂CO₃ (2 equiv.) at 0° C., after which the solution was allowed to warm up to room temperature. After stirring at r.t. for 10 minutes, an alkyl halide of interest (1.5 equiv.) was added, after which the reaction mixture was continued stirring at room temperature. After completion of the reaction was confirmed by TLC, water was added to the reaction mixture which was then extracted with ethyl acetate. The combined organic layers were washed with water, sat. brine, dried over anhydrous sodium sulfate and evaporated under reduced pressure to afford a crude residue. The obtained residual compound was purified by column chromatography over silica gel, eluting with EtOAc in hexane, affording the 4-alkoxy-2,6-difluorobenzonitrile of interest.

### Step (ii): 4-Alkoxy-2-fluoro-6-(piperazin-1-yl)benzonitrile

To a stirred solution of 4-alkoxy-2,6-difluorobenzonitrile (1 equiv.) in acetonitrile was added piperazine (5 equiv.), after which the resulting solution was stirred at 80° C. for 10-15 h. After completion of the reaction was confirmed by TLC, the reaction mixture was evaporated to dryness, water was added and the obtained solution was then extracted with EtOAc. The combined organic layers were washed with sat. brine, dried over anhydrous sodium sulfate and concentrated in vacuo. Subsequent purification via column chromatography (SiO₂, eluting with MeOH in CH₂Cl₂), afforded the targeted 4-alkoxy-2-fluoro-6-(piperazin-1-yl)benzonitrile.

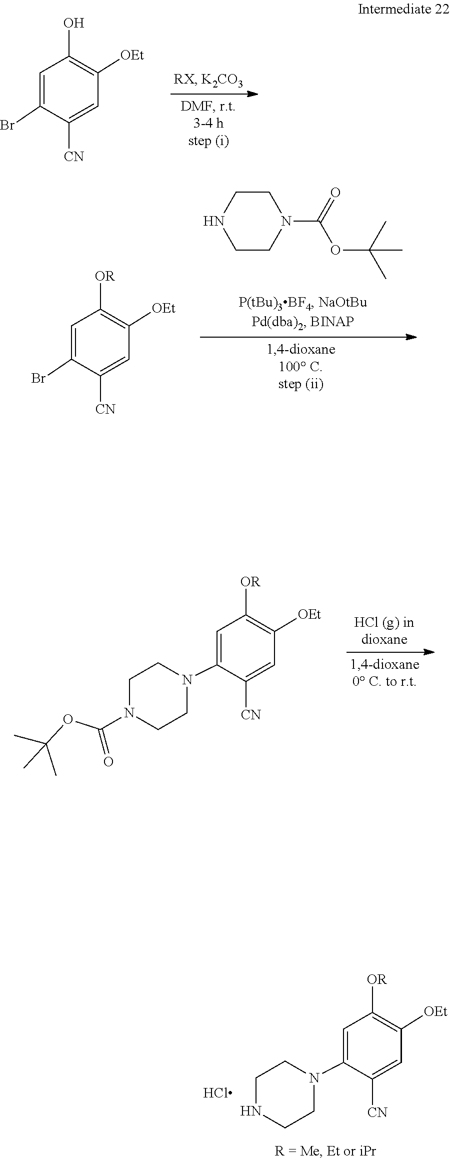

### Step (i): 4-Alkoxy-2-bromo-5-ethoxybenzonitrile

To a stirred solution of 2-bromo-5-ethoxy-4-hydroxybenzonitrile (1 equiv.) in DMF was added K₂CO₃ (2-2.5 equiv.) at 0° C., after which the solution was allowed to warm up to room temperature. After stirring at r.t. for 10 minutes, an alkyl halide of interest (1.5 equiv.) was added, after which the reaction mixture was kept stirring at room temperature. After completion of the reaction was confirmed by TLC, water was added to the reaction mixture which was then extracted with ethyl acetate. The combined organic layers were washed with water, sat. brine, dried over anhydrous sodium sulfate and evaporated under reduced pressure to afford a crude residue. The obtained residual compound was purified by column chromatography over silica gel, eluting with EtOAc in hexane, as such affording the 4-alkoxy-2-bromo-5-ethoxybenzonitrile of interest.

### Step (ii): 4-Alkoxy-3-ethoxy-6-(4-tert-butoxycarbonylpiperazin-1-yl)benzonitrile

To a stirred solution of 4-alkoxy-2-bromo-5-ethoxybenzonitrile (1 equiv.) in 1,4-dioxane was added tert-butyl piperazine-1-carboxylate (1.5 equiv), followed by NaOtBu (2-2.5 equiv.) and P(tBu)₃·BF₄ (1.5 equiv.) and the resultant mixture was bubbled with argon for 20 min. Then Pd(dba)₂ (0.05 equiv.) and BINAP (0.1 equiv.) were added after which the reaction was heated to 100° C. until completion of the reaction was observed by TLC. The reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (30-40% EtOAc in hexane) yielded the desired substituted nitrile.

### Step (iii): 4-Alkoxy-3-ethoxy-6-(piperazin-1-yl)benzonitrile hydrochloride

To a stirred solution of 4-alkoxy-3-ethoxy-6-(4-tert-butoxycarbonylpiperazin-1-yl)benzonitrile (1 equiv.) in 1,4-dioxane was added an excess of HCl (g) in dioxane at 0° C. The reaction was allowed to slowly warm up to room temperature and kept stirring at room temperature until complete conversion was confirmed by TLC. The reaction mixture was concentrated to dryness, followed by trituration in an appropriate solvent (e.g. diethyl ether). The obtained hydrochloride salt was used as such in the following nucleophilic substitution.

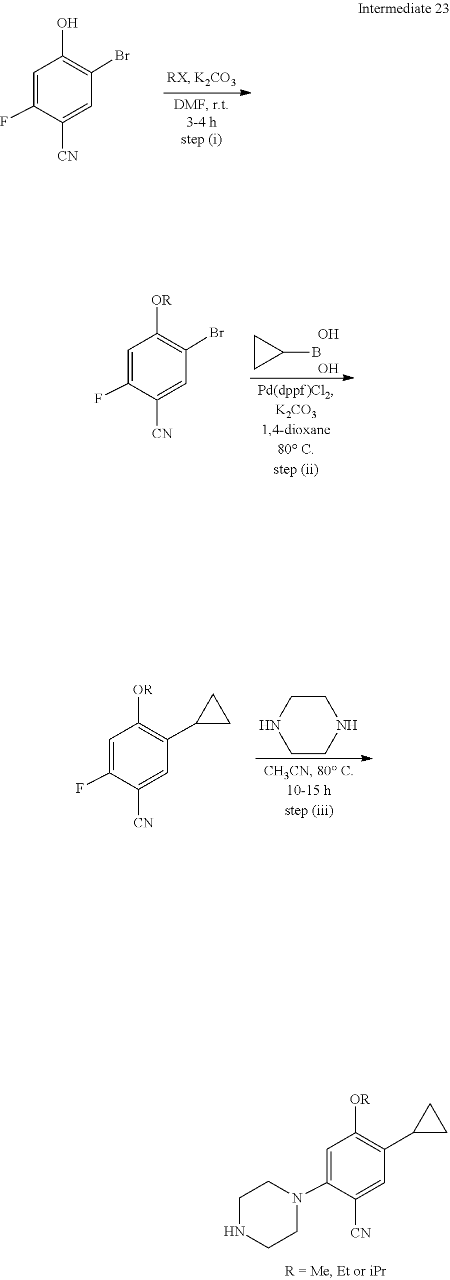

### Step (i): 4-Alkoxy-5-bromo-2-fluorobenzonitrile

To a stirred solution of 5-bromo-2-fluoro-4-hydroxybenzonitrile (1 equiv.) in DMF was added K₂CO₃ (2 equiv.) at 0° C., after which the solution was allowed to warm up to room temperature. After stirring at r.t. for 10 minutes, an alkyl halide of interest (1.5 equiv.) was added, after which the reaction mixture was left stirring at room temperature. After completion of the reaction was confirmed by TLC, water was added to the reaction mixture which was then extracted with ethyl acetate. The combined organic layers were washed with water, sat. brine, dried over anhydrous sodium sulfate and evaporated under reduced pressure to afford a crude residue. The obtained residual compound was purified by column chromatography over silica gel, eluting with EtOAc in hexane, affording the 4-alkoxy-5-bromo-2-fluorobenzonitrile of interest.

### Step (ii): 4-Alkoxy-5-cyclopropyl-2-fluorobenzonitrile

To a stirred solution of 4-alkoxy-5-bromo-2-fluorobenzonitrile (1 equiv., 0.371 mmol) in 1,4-dioxane (10 mL) was added cyclopropylboronic acid (1.2 equiv., 39 mg, 0.445 mmol), followed by K₂CO₃ (2.5 equiv., 128 mg, 0.927 mmol) and the resulting mixture was bubbled with argon for 20 min. Then Pd(dppf)Cl₂ (0.05 equiv., 14 mg, 0.019 mmol) was added after which the reaction was heated to 80° C. for 12 h. After the completion of reaction was confirmed by TLC and LC-MS, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (15-20% EtOAc in hexane) yielded the desired 4-alkoxy-5-cyclopropyl-2-fluorobenzonitrile.

### Step (iii): 4-Alkoxy-3-cyclopropyl-6-(piperazin-1-yl)benzonitrile

To a stirred solution of 4-alkoxy-5-cyclopropyl-2-fluorobenzonitrile (1 equiv.) in acetonitrile was added piperazine (5 equiv.), after which the resulting solution was stirred at 80° C. for 10-15 h. After completion of the reaction was confirmed by TLC, the reaction mixture was evaporated to dryness, water was added and the obtained solution was then extracted with EtOAc. The combined organic layers were washed with sat. brine, dried over anhydrous sodium sulfate and concentrated in vacuo. Subsequent purification via column chromatography (SiO₂, eluting with 20-30% EtOAc in hexane), afforded the targeted 4-alkoxy-3-cyclopropyl-6-(piperazin-1-yl)benzonitrile as a gummy solid.

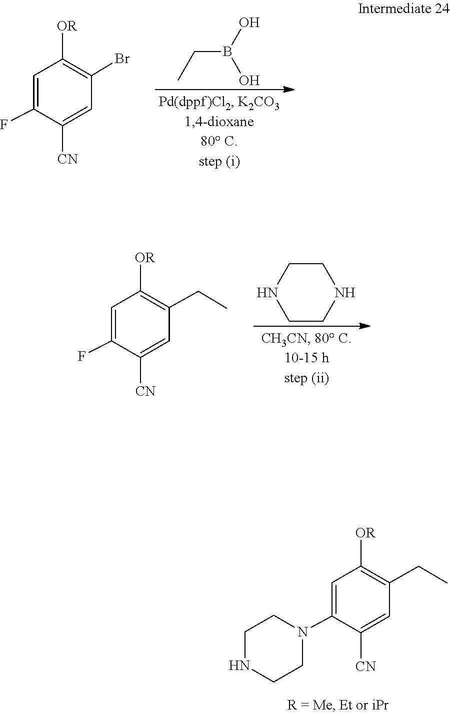

### Step (i): 4-Alkoxy-5-ethyl-2-fluorobenzonitrile

To a stirred solution of 4-alkoxy-5-bromo-2-fluorobenzonitrile (1 equiv., 0.371 mmol) in 1,4-dioxane (10 mL) was added ethylboronic acid (1.2 equiv., 33 mg, 0.445 mmol), followed by K₂CO₃ (2.5 equiv., 128 mg, 0.927 mmol) and the resulting mixture was bubbled with argon for 20 min. Then Pd(dppf)Cl₂ (0.05 equiv., 14 mg, 0.019 mmol) was added after which the reaction was heated to 80° C. for 12 h. After the completion of reaction was confirmed by TLC and LC-MS, the reaction mixture was evaporated under reduced pressure to remove the volatiles and the residue was re-dissolved with ethyl acetate and washed with water and sat. brine. Subsequent drying over anhydrous sodium sulfate and concentration in vacuo, followed by column chromatography over silica gel (15-20% EtOAc in hexane) yielded the desired 4-alkoxy-5-ethyl-2-fluorobenzonitrile.

### Step (iii): 4-Alkoxy-3-ethyl-6-(piperazin-1-yl)benzonitrile

To a stirred solution of 4-alkoxy-5-ethyl-2-fluorobenzonitrile (1 equiv.) in acetonitrile was added piperazine (5 equiv.), after which the resulting solution was stirred at 80° C. for 10-15 h. After completion of the reaction was confirmed by TLC, the reaction mixture was evaporated to dryness, diluted with water and extracted with EtOAc. The combined organic layers were washed with sat. brine, dried over anhydrous sodium sulfate and concentrated under reduced pressure. Subsequent purification via column chromatography (SiO₂, eluting with 20-30% EtOAc in hexane), afforded the targeted 4-alkoxy-3-ethyl-6-(piperazin-1-yl)benzonitrile.

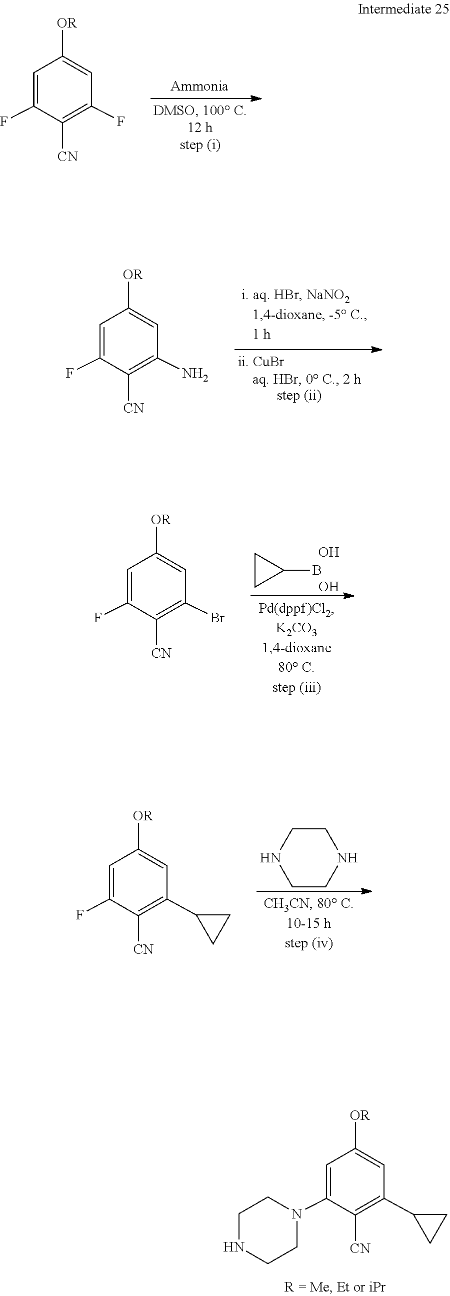

### Step (i): 2-Amino-4-alkoxy-6-fluorobenzonitrile

To a stirred solution of 4-alkoxy-2,6-difluorobenzonitrile of interest (1 equiv., 50.7 mmol) in DMSO (10 mL) in a sealed tube was added an excess of ammonia in 1,4-dioxane, after which the reaction mixture was brought to 100° C. for 12 h. After completion of the reaction was confirmed by TLC, the reaction mixture was diluted with water and extraction with diethyl ether was performed. The combined organic layers were washed with sat. brine, dried over anhydrous sodium sulfate and evaporated under reduced pressure to afford a crude residue. The obtained residual compound was purified via column chromatography over silica gel (eluting with 10-15% EtOAc in hexane), affording the 2-amino-4-alkoxy-6-fluorobenzonitrile of interest as an off-white solid.

### Step (ii): 4-Alkoxy-2-bromo-6-fluorobenzonitrile

To a stirred solution of 2-amino-4-alkoxy-6-fluorobenzonitrile (1 equiv., 12.88 mmol) in 1,4-dioxane (10 mL) was added aqueous HBr (25 mL) at 0° C., after which the solution was allowed to warm up to room temperature. After stirring at r.t. for 30 minutes, the reaction mixture was cooled down to −5° C. and a solution of NaNO₂ (1.2 equiv, 1.06 g, 15.4 mmol) in water (5 mL) was carefully added. Upon completion of the addition, the reaction mixture was kept stirring at −5° C. for an additional hour. Next, this reaction mixture was slowly transferred to a pre-cooled solution of CuBr (1.1 equiv., 2.03 g, 14.2 mmol) in aqueous HBr (5 mL), after which the combined solution was left stirring at 0° C. for 2 h. After completion of the reaction was confirmed by TLC, the reaction mixture was neutralized with sat. NaHCO₃ and extraction with ethyl acetate was performed. The combined organic layers were subsequently washed with water, sat. brine and dried over anhydrous sodium sulfate. Concentration under reduced pressure afforded a crude residue, which was further purified by column chromatography over silica gel, eluting with 1-2% EtOAc in hexane, affording the 4-alkoxy-2-bromo-6-fluorobenzonitrile of interest as a gummy liquid.

### Step (iii): 4-Alkoxy-2-cyclopropyl-6-fluorobenzonitrile

To a stirred solution of 4-alkoxy-2-bromo-6-fluorobenzonitrile (1 equiv., 1.93 mmol) in 1,4-dioxane (10 mL) was added cyclopropylboronic acid (1.2 equiv., 199 mg, 2.32 mmol), followed by K₂CO₃ (2.5 equiv., 667 mg, 4.83 mmol) and the resulting mixture was bubbled with argon for 20 min. Then Pd(dppf)Cl₂ (0.05 equiv., 70 mg, 0.097 mmol) was added after which the reaction was heated to 80° C. for 12 h. After the completion of reaction was confirmed by TLC and LC-MS, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (10-20% EtOAc in hexane) yielded the desired 4-alkoxy-2-cyclopropyl-6-fluorobenzonitrile.

### Step (iv): 4-Alkoxy-2-cyclopropyl-6-(piperazin-1-yl)benzonitrile

To a stirred solution of 4-alkoxy-2-cyclopropyl-6-fluorobenzonitrile (1 equiv., 1.36 mmol) in acetonitrile (10 mL) was added piperazine (5 equiv., 586 mg, 6.8 mmol), after which the resulting solution was stirred at 80° C. for 10-15 h. After completion of the reaction was confirmed by TLC, the reaction mixture was evaporated to dryness, water was added and the obtained solution was extracted with EtOAc. The combined organic layers were washed with sat. brine, dried over anhydrous sodium sulfate and concentrated in vacuo. Subsequent purification via column chromatography (SiO₂, eluting with 2-4% MeOH in CH₂Cl₂), afforded the targeted 4-alkoxy-2-cyclopropyl-6-(piperazin-1-yl)benzonitrile of interest.

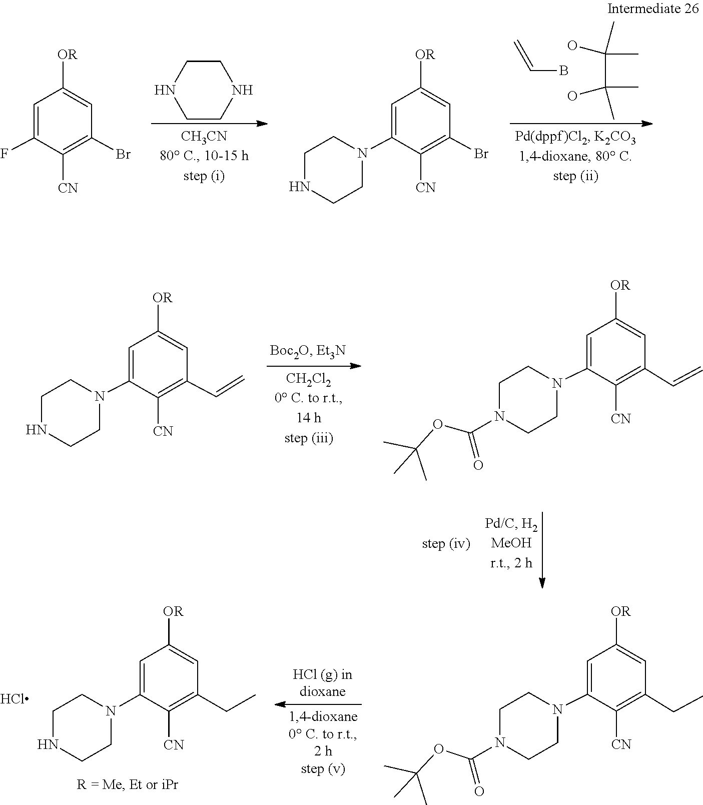

### Step (i): 4-Alkoxy-2-bromo-6-(piperazin-1-yl)benzonitrile

After addition of piperazine (5 equiv., 831 mg, 9.65 mmol) to a stirred solution of 4-alkoxy-2-bromo-6-fluorobenzonitrile (1 equiv., 1.93 mmol) in acetonitrile (15 mL), the reaction was continued at 80° C. for 10-15 h. After the completion of the reaction was confirmed by TLC, the reaction mixture was evaporated to dryness. Next, the crude residue was purified by column chromatography over silica gel, eluting using 24% of MeOH in CH₂Cl₂, to afford the envisaged 4-alkoxy-2-bromo-6-(piperazin-1-yl)benzonitrile as a gummy liquid.

### Step (ii): 4-Alkoxy-2-(piperazin-1-yl)-6-vinylbenzonitrile

To a stirred solution of 4-alkoxy-2-bromo-6-(piperazin-1-yl)benzonitrile (1 equiv., 1.23 mmol) in 1,4-dioxane (10 mL) was added 4,4,5,5-tetramethyl-2-vinyl-1,3,2-dioxaborolane (1.2 equiv., 228 mg, 1.48 mmol), followed by K₂CO₃ (2.5 equiv., 424 mg, 3.07 mmol) and the resultant mixture was bubbled with argon for 20 min. Then Pd(dppf)Cl₂ (0.05 equiv., 45 mg, 0.062 mmol) was added after which the reaction was heated to 80° C. for 12 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure afforded the desired 4-alkoxy-2-(piperazin-1-yl)-6-vinylbenzonitrile which was taken to the next step without additional purification.

### Step (iii): tert-Butyl 4-(5-alkoxy-2-cyano-3-vinylphenyl)piperazine-1-carboxylate

To an ice-cold solution of 4-alkoxy-2-(piperazin-1-yl)-6-vinylbenzonitrile in CH₂Cl₂ (25 mL) was added Et₃N (2.5 equiv., 0.43 mL, 3.08 mmol). The solution was stirred at 0° C. for 10 minutes, after which Boc-anhydride (1.5 equiv., 403 mg, 1.85 mmol) was added and the reaction was allowed to continue at room temperature for an additional 14 hours. After completion of the reaction was confirmed by TLC, cold water was added to the reaction mixture which was then extracted with CH₂Cl₂. The combined organic layers were washed with sat. brine, dried over anhydrous sodium sulfate and evaporated under reduced pressure. The crude obtained was purified by column chromatography over silica gel (20-30% EtOAc in hexane) to afford tert-butyl 4-(5-alkoxy-2-cyano-3-vinylphenyl)piperazine-1-carboxylate as a colorless gum.

### Step (iv): tert-Butyl 4-(5-alkoxy-2-cyano-3-ethylphenyl)piperazine-1-carboxylate

A stirred solution of tert-butyl 4-(5-alkoxy-2-cyano-3-vinylphenyl)piperazine-1-carboxylate (1 equiv., 0.808 mmol) in MeOH (20 mL) was hydrogenated over 10% Pd/C (70 mg) under 5 Kg/cm² H₂ pressure using a Parr hydrogenator for 2 h at ambient temperature. After confirming the completion of reaction by LC-MS, the reaction mixture was filtered through a Celite® bed and was concentrated in vacuo, yielding the desired compound as a colorless gum.

### Step (v): 4-Alkoxy-2-ethyl-6-(piperazin-1-yl)benzonitrile hydrochloride

tert-Butyl 4-(5-alkoxy-2-cyano-3-ethylphenyl)piperazine-1-carboxylate (1 equiv., 0.72 mmol) was dissolved in 4M HCl in 1,4-dioxane (10 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford the targeted 4-alkoxy-2-ethyl-6-(piperazin-1-yl)benzonitrile hydrochloride as an off-white solid.

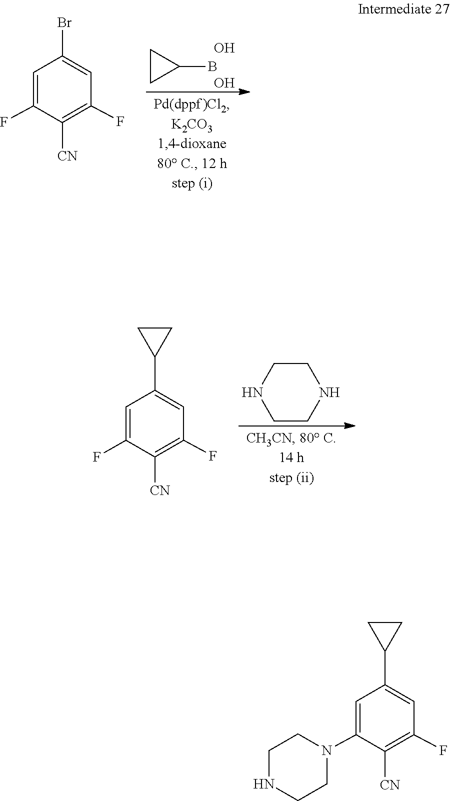

### Step (i): 4-Cyclopropyl-2,6-difluorobenzonitrile

To a stirred solution of 4-bromo-2,6-difluorobenzonitrile (1 equiv., 91, mg, 0.417 mmol) in 1,4-dioxane (10 mL) was added cyclopropylboronic acid (1.2 equiv., 43 mg, 0.50 mmol), followed by K₂CO₃ (2.5 equiv., 144 mg, 1.04 mmol) and the resulting mixture was bubbled with argon for 20 min. Then Pd(dppf)Cl₂ (0.05 equiv., 15 mg, 0.021 mmol) was added after which the reaction was heated to 80° C. for 12 h. After the completion of reaction was confirmed by TLC and LC-MS, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (15-20% EtOAc in hexane), yielded the desired 4-cyclopropyl-2,6-difluorobenzonitrile as a pale-yellow solid (64 mg, 85%).

### Step (ii): 2-Fluoro-4-cyclopropyl-6-(piperazin-1-yl)benzonitrile

To a stirred solution of 4-cyclopropyl-2,6-difluorobenzonitrile (1 equiv., 60 mg, 0.335 mmol) in acetonitrile (5 mL) was added piperazine (5 equiv., 144 mg, 1.67 mmol), after which the resulting solution was stirred at 80° C. for 14 h. After completion of the reaction was confirmed by TLC, the reaction mixture was evaporated to dryness, water was added and the obtained solution extracted with EtOAc. The combined organic layers were washed with sat. brine, dried over anhydrous sodium sulfate and concentrated in vacuo. Subsequent purification via column chromatography (SiO₂, eluting with 2-4% MeOH in CH₂Cl₂), afforded the targeted 2-fluoro-4-cyclopropyl-6-(piperazin-1-yl)benzonitrile as a gummy liquid (59 mg, 72%).

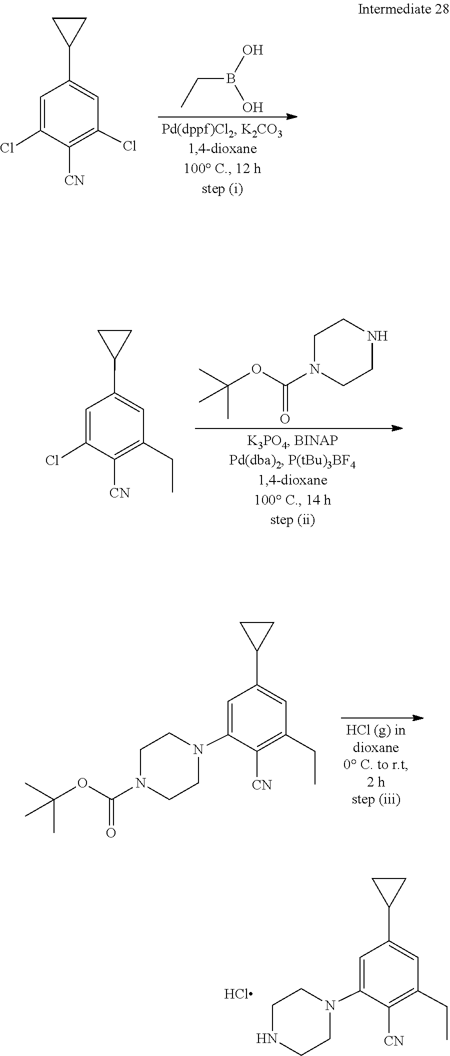

### Step (i): 2-Chloro-4-cyclopropyl-6-ethylbenzonitrile

To a stirred solution of 2,6-dichloro-4-cyclopropylbenzonitrile (0.37 g, 1.745 mmol) in 1,4-dioxane (20 mL) was added ethylboronic acid (0.152 g, 2.06 mmol), followed by K₂CO₃ (0.593 g, 4.36 mmol) and the resultant mixture was bubbled with argon for 20 min. Then Pd(dppf)Cl₂ (0.064 g, 0.0873 mmol) was added after which the reaction was heated to 100° C. for 12 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (15-18% EtOAc in hexane), afforded 2-chloro-4-cyclopropyl-6-ethylbenzonitrile as a gummy liquid (0.29 g, 77%).

### Step (ii): tert-Butyl 4-(2-cyano-5-cyclopropyl-3-ethylphenyl)piperazine-1-carboxylate

To a stirred solution of 2-chloro-4-cyclopropyl-6-ethylbenzonitrile (0.3 g, 1.41 mmol) in 1,4-dioxane (20 mL) was added tert-butyl piperazine-1-carboxylate (0.314 g, 1.69 mmol), followed by K₃PO₄ (0.731 g, 3.45 mmol) and BINAP (0.034 g, 0.0552 mmol). The resultant mixture was bubbled with argon for 20 min. Then P(tBu)₃·BF₄ (0.016 g, 0.0552 mmol) followed by Pd(dba)₂ (0.040 g, 0.069 mmol) were added after which the reaction was heated to 100° C. for 14 h. After the completion of reaction was confirmed by TLC, the reaction mixture was evaporated in vacuo to remove the volatiles and the residue was re-dissolved with ethyl acetate and washed with water and sat. brine. Drying over anhydrous sodium sulfate and concentration in vacuo, followed by column chromatography over silica gel (30-35% EtOAc in hexane), afforded the desired compound as a pale-yellow gum (0.29 g, 60%).

### Step (iii): 4-Cyclopropyl-2-ethyl-6-(piperazin-1-yl)benzonitrile hydrochloride

tert-Butyl 4-(2-cyano-5-cyclopropyl-3-ethylphenyl)piperazine-1-carboxylate (0.29 g, 0.816 mmol) was dissolved in 4M HCl in 1,4-dioxane (10 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford 4-cyclopropyl-2-ethyl-6-(piperazin-1-yl)benzonitrile hydrochloride as an off-white solid (0.20 g, 80%).

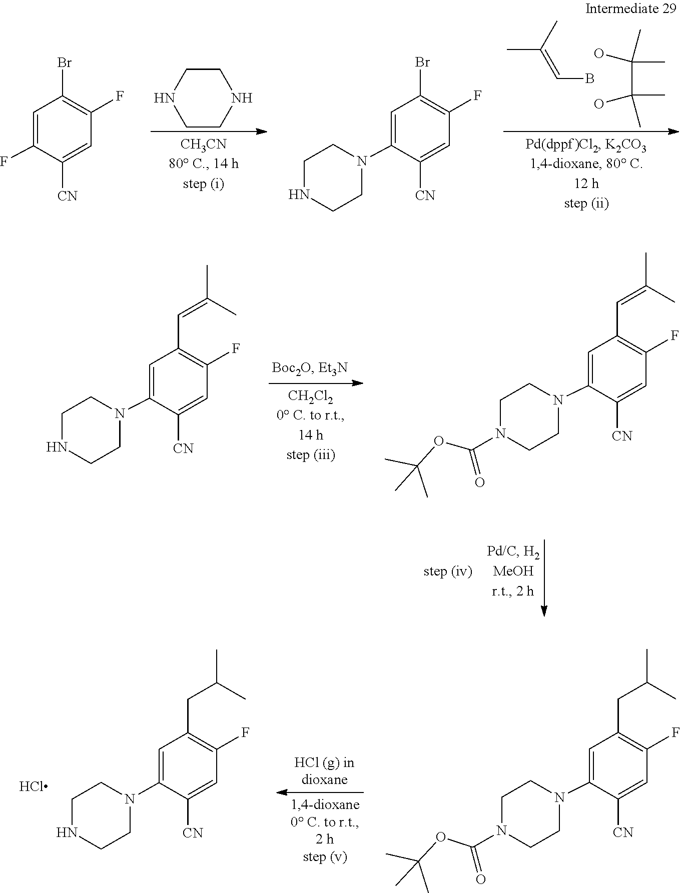

### Step (i): 4-Bromo-5-fluoro-2-(piperazin-1-yl)benzonitrile

After addition of piperazine (1.97 g, 22.9 mmol) to a stirred solution of 4-bromo-2,5-difluorobenzonitrile (1.0 g, 4.58 mmol) in acetonitrile (25 mL), the reaction was continued at 80° C. for 14 h. After the completion of the reaction was confirmed by TLC, the reaction mixture was evaporated to dryness. Next, the crude residue was purified by column chromatography over silica gel, eluting using 2-4% of MeOH in CH₂Cl₂ to afford 4-bromo-5-fluoro-2-(piperazin-1-yl)benzonitrile as gummy liquid (1.03 g, 79%).

### Step (ii): 5-Fluoro-4-(2-methylprop-1-en-1-yl)-2-(piperazin-1-yl)benzonitrile

To a stirred solution of 4-bromo-5-fluoro-2-(piperazin-1-yl)benzonitrile (1.01 g, 3.55 mmol) in 1,4-dioxane (30 mL) was added 4,4,5,5-tetramethyl-2-(2-methylprop-1-en-1-yl)-1,3,2-dioxaborolane (776 mg, 4.26 mmol), followed by K₂CO₃ (1.22 g, 8.88 mmol) and the resultant mixture was bubbled with argon for 20 min. Then Pd(dppf)Cl₂ (130 mg, 0.178 mmol) was added after which the reaction was heated to 80° C. for 12 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure afforded 5-fluoro-4-(2-methylprop-1-en-1-yl)-2-(piperazin-1-yl)benzonitrile which was taken to the next step without additional purification.

### Step (iii): tert-Butyl 4-(2-cyano-4-fluoro-5-(2-methylprop-1-en-1-yl)phenyl)piperazine-1-carboxylate

To an ice-cold solution of 5-fluoro-4-(2-methylprop-1-en-1-yl)-2-(piperazin-1-yl)benzonitrile in CH₂Cl₂ (50 mL) was added Et₃N (1.24 mL, 8.88 mmol). The solution was stirred at 0° C. for 10 minutes, after which Boc-anhydride (1.16 g, 5.33 mmol) was added and the reaction was allowed to continue at room temperature for an additional 14 hours. After completion of the reaction was confirmed by TLC, cold water was added to the reaction mixture which was then extracted with CH₂Cl₂. The combined organic layers were washed with sat. brine, dried over anhydrous sodium sulfate and evaporated under reduced pressure. The crude obtained was purified by column chromatography over silica gel (20-30% EtOAc in hexane) to afford tert-butyl 4-(2-cyano-4-fluoro-5-(2-methylprop-1-en-1-yl)phenyl)piperazine-1-carboxylate as a colorless gum (0.85 g, 85% over 2 steps).

### Step (iv): tert-Butyl 4-(2-cyano-4-fluoro-5-isobutylphenyl)piperazine-1-carboxylate

A stirred solution of tert-butyl 4-(2-cyano-4-fluoro-5-(2-methylprop-1-en-1-yl)phenyl)piperazine-1-carboxylate (0.85 g, 2.36 mmol) in MeOH (30 mL) was hydrogenated over 10% Pd/C (0.2 g) under 5 Kg/cm² H₂ pressure using a Parr hydrogenator for 2 h at ambient temperature. After confirming the completion of reaction by LC-MS, the reaction mixture was filtered through a Celite® bed and was concentrated in vacuo, yielding the desired compound as a colorless gum (0.78 g, 91%).

### Step (v): 5-Fluoro-4-isobutyl-2-(piperazin-1-yl)benzonitrile hydrochloride

tert-Butyl 4-(2-cyano-4-fluoro-5-isobutylphenyl)piperazine-1-carboxylate (0.18 g, 0.336 mmol) was dissolved in 4M HCl in 1,4-dioxane (10 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford 5-fluoro-4-isobutyl-2-(piperazin-1-yl)benzonitrile hydrochloride as an off-white solid (0.12 g, 81%).

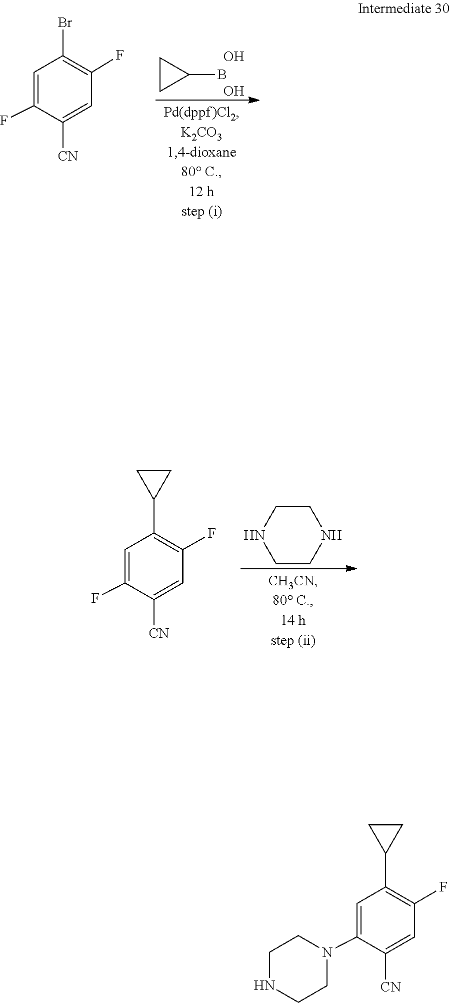

### Step (i): 4-Cyclopropyl-2,5-difluorobenzonitrile

To a stirred solution of 4-bromo-2,5-difluorobenzonitrile (1 equiv., 2.15 g, 9.86 mmol) in 1,4-dioxane (120 mL) was added cyclopropylboronic acid (1.2 equiv., 1.02 g, 11.8 mmol), followed by K₂CO₃ (2.5 equiv., 3.41 g, 24.6 mmol) and the resulting mixture was bubbled with argon for 20 min. Then Pd(dppf)Cl₂ (0.05 equiv., 361 mg, 0.493 mmol) was added after which the reaction was heated to 80° C. for 12 h. After the completion of reaction was confirmed by TLC and LC-MS, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (15-20% EtOAc in hexane), yielded the desired 4-cyclopropyl-2,5-difluorobenzonitrile as an off-white solid (1.57 g, 89%).

### Step (ii): 5-Fluoro-4-cyclopropyl-2-(piperazin-1-yl)benzonitrile

To a stirred solution of 4-cyclopropyl-2,5-difluorobenzonitrile (1 equiv., 1.50 g, 8.37 mmol) in acetonitrile (120 mL) was added piperazine (5 equiv., 3.61 g, 41.9 mmol), after which the resulting solution was stirred at 80° C. for 14 h. After completion of the reaction was confirmed by TLC, the reaction mixture was evaporated to dryness, water was added and the resulting solution was extracted with EtOAc. The combined organic layers were washed with sat. brine, dried over anhydrous sodium sulfate and concentrated in vacuo. Subsequent purification via column chromatography (SiO₂, eluting with 24% MeOH in CH₂Cl₂), delivered the desired 5-fluoro-4-cyclopropyl-2-(piperazin-1-yl)benzonitrile as a gummy liquid (1.60 g, 78%).

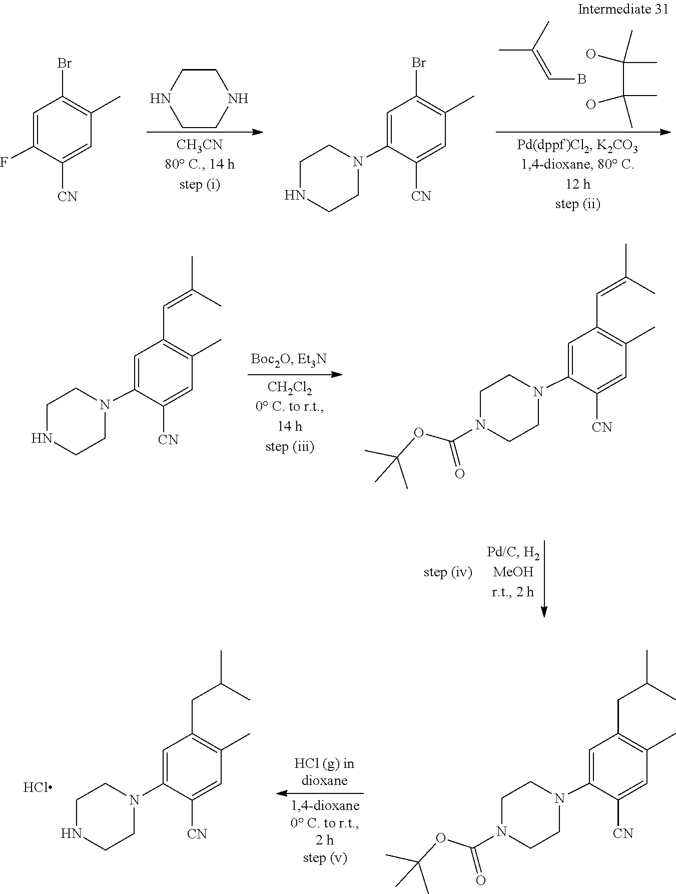

### Step (i): 4-Bromo-5-methyl-2-(piperazin-1-yl)benzonitrile

After addition of piperazine (2.01 g, 23.36 mmol) to a stirred solution of 4-bromo-2-fluoro-5-methylbenzonitrile (1 g, 4.67 mmol) in acetonitrile (30 mL), the reaction was continued at 80° C. for 14 h. After the completion of the reaction was confirmed by TLC, the reaction mixture was evaporated to dryness. Next, the crude residue was purified by column chromatography over silica gel eluting with 2-4% of MeOH in CH₂Cl₂ to afford 4-bromo-5-methyl-2-(piperazin-1-yl)benzonitrile as gummy liquid (0.86 g, 66%).

### Step (ii): 5-Methyl-4-(2-methylprop-1-en-1-yl)-2-(piperazin-1-yl)benzonitrile

To a stirred solution of 4-bromo-5-methyl-2-(piperazin-1-yl)benzonitrile (0.85 g, 3.03 mmol) in 1,4-dioxane (25 mL) was added 4,4,5,5-tetramethyl-2-(2-methylprop-1-en-1-yl)-1,3,2-dioxaborolane (663 mg, 3.64 mmol), followed by K₂CO₃ (1.04 g, 7.58 mmol) and the resultant mixture was bubbled with argon for 20 min. Then Pd(dppf)Cl₂ (111 mg, 0.151 mmol) was added after which the reaction was heated to 80° C. for 12 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure afforded 5-methyl-4-(2-methylprop-1-en-1-yl)-2-(piperazin-1-yl)benzonitrile which was taken to the next step without additional purification.

### Step (iii): tert-Butyl 4-(2-cyano-4-methyl-5-(2-methylprop-1-en-1-yl)phenyl)piperazine-1-carboxylate

To an ice-cold solution of 5-methyl-4-(2-methylprop-1-en-1-yl)-2-(piperazin-1-yl)benzonitrile in CH₂Cl₂ (50 mL) was added Et₃N (1.06 mL, 7.58 mmol). The solution was stirred at 0° C. for 10 minutes, after which Boc-anhydride (0.992 g, 4.54 mmol) was added and the reaction was allowed to continue at room temperature for an additional 14 hours. After completion of the reaction was confirmed by TLC, cold water was added to the reaction mixture which was then extracted with CH₂Cl₂. The combined organic layers were washed with sat. brine, dried over anhydrous sodium sulfate and evaporated under reduced pressure. The crude obtained was purified by column chromatography over silica gel (25-30% EtOAc in hexane) to afford tert-butyl 4-(2-cyano-4-methyl-5-(2-methylprop-1-en-1-yl)phenyl)piperazine-1-carboxylate as a colorless gum (0.65 g, 63% over 2 steps).

### Step (iv): tert-Butyl 4-(2-cyano-5-isobutyl-4-methylphenyl)piperazine-1-carboxylate

A stirred solution of tert-butyl 4-(2-cyano-4-methyl-5-(2-methylprop-1-en-1-yl)phenyl)piperazine-1-carboxylate (0.65 g, 1.83 mmol) in MeOH (30 mL) was hydrogenated over 10% Pd/C (0.2 g) under 5 Kg/cm² H₂ pressure using a Parr hydrogenator for 2 h at ambient temperature. After confirming the completion of reaction by LC-MS, the reaction mixture was filtered through a Celite® bed and was concentrated in vacuo, yielding the desired compound as a colorless gum (0.54 g, 83%).

### Step (v): 4-Isobutyl-5-methyl-2-(piperazin-1-yl)benzonitrile hydrochloride

tert-Butyl 4-(2-cyano-5-isobutyl-4-methylphenyl)piperazine-1-carboxylate (0.25 g, 0.70 mmol) was dissolved in 4M HCl in 1,4-dioxane (10 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford 4-isobutyl-5-methyl-2-(piperazin-1-yl)benzonitrile hydrochloride as an off-white solid (0.15 g, 75%).

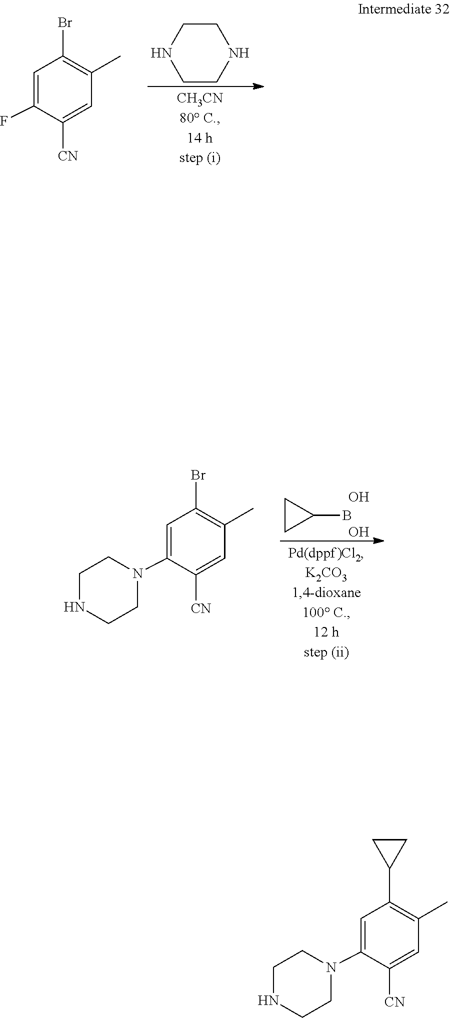

### Step (i): 4-Bromo-5-methyl-2-(piperazin-1-yl)benzonitrile

To a stirred solution of 4-bromo-2-fluoro-5-methylbenzonitrile (1 equiv., 200 mg, 0.934 mmol) in acetonitrile (15 mL) was added piperazine (5 equiv., 402 mg, 4.67 mmol), after which the resulting solution was stirred at 80° C. for 14 h. After completion of the reaction was confirmed by TLC, the reaction mixture was evaporated to dryness, water was added and the resulting solution was extracted with EtOAc. The combined organic layers were washed with sat. brine, dried over anhydrous sodium sulfate and concentrated in vacuo. Subsequent purification via column chromatography (SiO₂, eluting with 2-4% MeOH in CH₂Cl₂), delivered 4-bromo-5-methyl-2-(piperazin-1-yl)benzonitrile as a gummy liquid (210 mg, 80%).

### Step (ii): 4-Cyclopropyl-5-methyl-2-(piperazin-1-yl)benzonitrile

To a stirred solution of 4-bromo-5-methyl-2-(piperazin-1-yl)benzonitrile (1 equiv., 200 mg, 0.714 mmol) in 1,4-dioxane (10 mL) was added cyclopropylboronic acid (1.2 equiv., 74 mg, 0.857 mmol), followed by K₂CO₃ (2.5 equiv., 247 mg, 1.78 mmol) and the resulting mixture was bubbled with argon for 20 min. Then Pd(dppf)Cl₂ (0.05 equiv., 26 mg, 0.036 mmol) was added after which the reaction was heated to 100° C. for 12 h. After the completion of reaction was confirmed by TLC and LC-MS, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (25-30% EtOAc in hexane), afforded the targeted 4-cyclopropyl-5-methyl-2-(piperazin-1-yl)benzonitrile as a gummy liquid (150 mg, 87%).

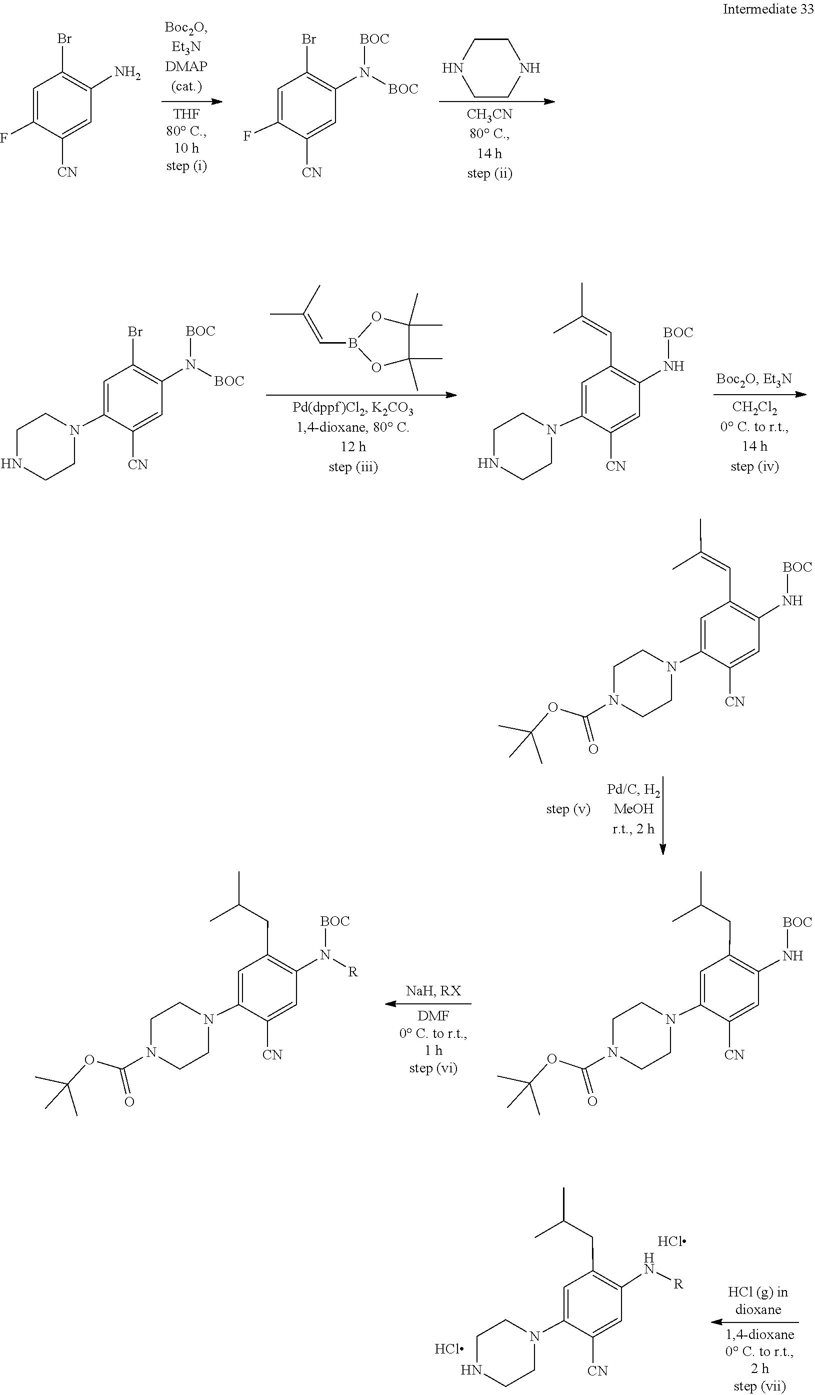

### Step (i): N,N-di-Boc-protected 2-bromo-5-cyano-4-fluoroaniline

To a stirred solution of 5-amino-4-bromo-2-fluorobenzonitrile (2.0 g, 9.38 mmol) in THE (50 mL) was added Et₃N (2.63 mL, 18.78 mmol), followed by a catalytic amount of DMAP and Boc-anhydride (4.1 g, 18.78 mmol). Upon completion of the addition, the reaction mixture was brought to 80° C. and was allowed to continue at 80° C. for 10 h. After completion of the reaction was confirmed by TLC, cold water was added to the reaction which was extracted with EtOAc. The combined organic layers were washed with sat. brine, dried over anhydrous sodium sulfate and evaporated under reduced pressure. The crude obtained was purified by column chromatography over silica gel (10-15% EtOAc in hexane) to afford the targeted di-Boc-protected aniline as an off-white solid (2.67 g, 69%).

### Step (ii): N,N-di-Boc-protected 2-bromo-5-cyano-4-(piperazin-1-yl)aniline

After addition of piperazine (2.21 g, 25.72 mmol) to a stirred solution of N,N-di-Boc-protected 2-bromo-5-cyano-4-fluoroaniline (2.67 g, 6.43 mmol) in acetonitrile (100 mL), the reaction was continued at 80° C. for 14 h. After the completion of the reaction was confirmed by TLC, the reaction mixture was evaporated to dryness. Next, the crude residue was purified by column chromatography over silica gel eluting with 2-4% of MeOH in CH₂Cl₂, to afford N,N-di-Boc-protected 2-bromo-5-cyano-4-(piperazin-1-yl)aniline as gummy liquid (2.51 g, 81%).

### Step (iii): tert-Butyl (5-cyano-2-(2-methylprop-1-en-1-yl)-4-(piperazin-1-yl)phenyl) carbamate

To a stirred solution of N,N-di-Boc-protected 2-bromo-5-cyano-4-(piperazin-1-yl)aniline (2.50 g, 5.19 mmol) in 1,4-dioxane (50 mL) was added 4,4,5,5-tetramethyl-2-(2-methylprop-1-en-1-yl)-1,3,2-dioxaborolane (1.14 g, 6.23 mmol), followed by K₂CO₃ (11.9 g, 12.98 mmol) and the resultant mixture was bubbled with argon for 20 min. Then Pd(dppf)Cl₂ (190 mg, 0.26 mmol) was added after which the reaction was heated to 80° C. for 12 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure afforded tert-butyl (5-cyano-2-(2-methylprop-1-en-1-yl)-4-(piperazin-1-yl)phenyl)carbamate which was taken to the next step without additional purification.

### Step (iv): tert-Butyl 4-(4-((tert-butoxycarbonyl)amino)-2-cyano-5-(2-methylprop-1-en-1-yl)phenyl)piperazine-1-carboxylate

To an ice-cold solution of tert-butyl (5-cyano-2-(2-methylprop-1-en-1-yl)-4-(piperazin-1-yl)phenyl)carbamate in CH₂Cl₂ (100 mL) was added Et₃N (1.81 mL, 12.98 mmol). The solution was stirred at 0° C. for 10 minutes, after which Boc-anhydride (1.70 g, 7.78 mmol) was added and the reaction was allowed to continue at room temperature for an additional 14 hours. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The crude obtained was purified by column chromatography over silica gel (15-20% EtOAc in hexane) to afford tert-butyl 4-(4-((tert-butoxycarbonyl)amino)-2-cyano-5-(2-methylprop-1-en-1-yl)phenyl)piperazine-1-carboxylate as a colorless gum (1.62 g, 70% over 2 steps).

### Step (v): tert-Butyl 4-(4-((tert-butoxycarbonyl)amino)-2-cyano-5-isobutylphenyl)piperazine-1-carboxylate

A stirred solution of tert-butyl 4-(4-((tert-butoxycarbonyl)amino)-2-cyano-5-(2-methylprop-1-en-1-yl)phenyl)piperazine-1-carboxylate (1.61 g, 3.53 mmol) in MeOH (50 mL) was hydrogenated over 10% Pd/C (0.3 g) under 5 Kg/cm² H₂ pressure using a Parr hydrogenator for 2 h at ambient temperature. After confirming the completion of reaction by LC-MS, the reaction mixture was filtered through a Celite® bed and was concentrated in vacuo, yielding the desired compound as a colorless gum (1.48 g, 92%).

### Step (vi): tert-Butyl 4-(4-((tert-butoxycarbonyl)(alkyl)amino)-2-cyano-5-isobutylphenyl)piperazine-1-carboxylate

To a stirred suspension of NaH (21 mg, 0.524 mmol) in DMF (5 mL) at 0° C. was added a solution of tert-butyl 4-(4-((tert-butoxycarbonyl)amino)-2-cyano-5-isobutylphenyl)piperazine-1-carboxylate (200 mg, 0.436 mmol) in DMF (5 mL), after which the resulting mixture was allowed to warm up to room temperature. After stirring at r.t. for 30 minutes, the suspension was cooled down again to 0° C. and an alkyl halide of interest (0.436 mmol) was added, after which the reaction mixture was left stirring at room temperature for one hour. After completion of the reaction was confirmed by TLC and LC-MS, the reaction mixture was quenched with ice water and an extraction with ethyl acetate was performed. The combined organic layers were washed with water, sat. brine, dried over anhydrous sodium sulfate and evaporated under reduced pressure to afford a crude residue. The obtained residual compound was purified by column chromatography over silica gel, eluting with 25-35% EtOAc in hexane, affording the targeted compound of interest as a colorless gum.

### Step (vii): 5-Alkylamino-4-isobutyl-2-(piperazin-1-yl)benzonitrile hydrochloride

tert-Butyl-4-(4-((tert-butoxycarbonyl)(alkyl)amino)-2-cyano-5-isobutylphenyl)piperazine-1-carboxylate (0.32 mmol) was dissolved in 4M HCl in 1,4-dioxane (5 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford the envisaged 5-alkylamino-4-isobutyl-2-(piperazin-1-yl)benzonitrile hydrochloride as an off-white solid.

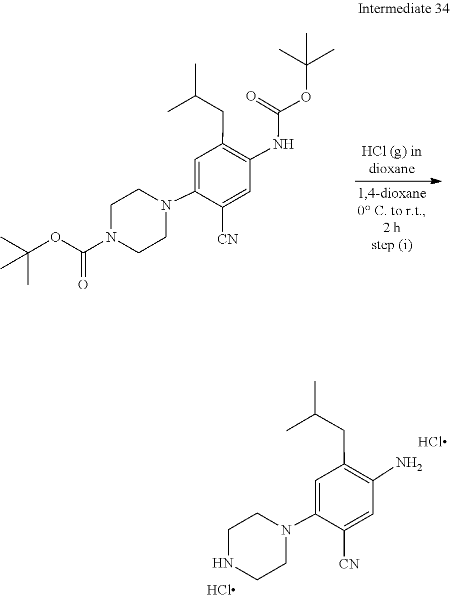 [SMILES: CC(C)Cc1cc(N2CCN(C(=O)OC(C)(C)C)CC2)c(C#N)cc1NC(=O)OC(C)(C)C ; CC(C)Cc1cc(N2CCNCC2)c(C#N)cc1N]

### Step (i): 5-Amino-4-isobutyl-2-(piperazin-1-yl)benzonitrile dihydrochloride

tert-Butyl-4-(4-((tert-butoxycarbonyl)amino)-2-cyano-5-isobutylphenyl)piperazine-1-carboxylate (220 mg, 0.48 mmol) was dissolved in 4M HCl in 1,4-dioxane (10 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford the envisaged 5-amino-4-isobutyl-2-(piperazin-1-yl)benzonitrile dihydrochloride as an off-white solid (0.12 g, 76%).

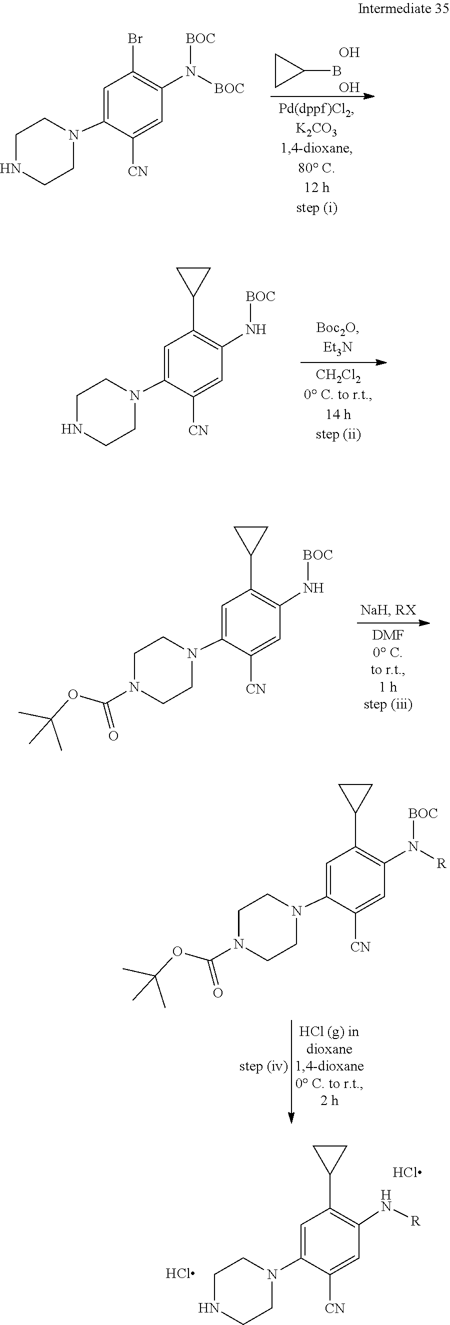

### Step (i): tert-Butyl (5-cyano-2-cyclopropyl-4-(piperazin-1-yl)phenyl)carbamate

To a stirred solution of N,N-di-Boc-protected 2-bromo-5-cyano-4-(piperazin-1-yl)aniline (144 mg, 0.30 mmol) in 1,4-dioxane (10 mL) was added cyclopropylboronic acid (1.2 equiv., 31 mg, 0.36 mmol), followed by K₂CO₃ (104 mg, 0.75 mmol) and the resultant mixture was bubbled with argon for 20 min. Then Pd(dppf)Cl₂ (11 mg, 0.015 mmol) was added after which the reaction was heated to 80° C. for 12 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure afforded tert-butyl (5-cyano-2-cyclopropyl-4-(piperazin-1-yl)phenyl)carbamate which was taken to the next step without additional purification.

### Step (ii): tert-Butyl 4-(4-((tert-butoxycarbonyl)amino)-2-cyano-5-cyclopropylphenyl)piperazine-1-carboxylate

To an ice-cold solution of tert-butyl (5-cyano-2-cyclopropyl-4-(piperazin-1-yl)phenyl)carbamate in CH₂Cl₂ (10 mL) was added Et₃N (0.11 mL, 0.75 mmol). The solution was stirred at 0° C. for 10 minutes, after which Boc-anhydride (98 mg, 0.45 mmol) was added and the reaction was allowed to continue at room temperature for an additional 14 hours. After completion of the reaction was confirmed by TLC, cold water was added to the reaction mixture which was extracted with CH₂Cl₂. The combined organic layers were washed with sat. brine, dried over anhydrous sodium sulfate and evaporated under reduced pressure. The crude obtained was purified by column chromatography over silica gel (15-20% EtOAc in hexane) to afford tert-butyl 4-(4-((tert-butoxycarbonyl)amino)-2-cyano-5-cyclopropylphenyl)piperazine-1-carboxylate as a colorless gum (110 mg, 82% over 2 steps).

### Step (iii): tert-Butyl 4-(4-((tert-butoxycarbonyl)(alkyl)amino)-2-cyano-5-cyclopropylphenyl)piperazine-I-carboxylate

To a stirred suspension of NaH (12 mg, 0.294 mmol) in DMF (3 mL) at 0° C. was added a solution of tert-butyl 4-(4-((tert-butoxycarbonyl)amino)-2-cyano-5-cyclopropylphenyl)piperazine-1-carboxylate (108 mg, 0.245 mmol) in DMF (5 mL), after which the resulting mixture was allowed to warm up to room temperature. After stirring at r.t. for 30 minutes, the suspension was cooled down again to 0° C. and an alkyl halide of interest (0.294 mmol) was added, after which the reaction mixture was left stirring at room temperature for one hour. After completion of the reaction was confirmed by TLC and LC-MS, the reaction mixture was quenched with ice water and an extraction with ethyl acetate was performed. The combined organic layers were washed with water, sat. brine, dried over anhydrous sodium sulfate and evaporated under reduced pressure to afford a crude residue. The obtained residual compound was purified by column chromatography over silica gel, eluting with 25-35% EtOAc in hexane, affording the targeted compound of interest as a colorless gum.

### Step (iv): 5-Alkylamino-4-cyclopropyl-2-(piperazin-1-yl)benzonitrile dihydrochloride

tert-Butyl-4-(4-((tert-butoxycarbonyl)(alkyl)amino)-2-cyano-5-cyclopropylphenyl) piperazine-1-carboxylate (0.184 mmol) was dissolved in 4M HCl in 1,4-dioxane (5 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford the envisaged 5-alkylamino-4-cyclopropyl-2-(piperazin-1-yl)benzonitrile dihydrochloride as an off-white solid.

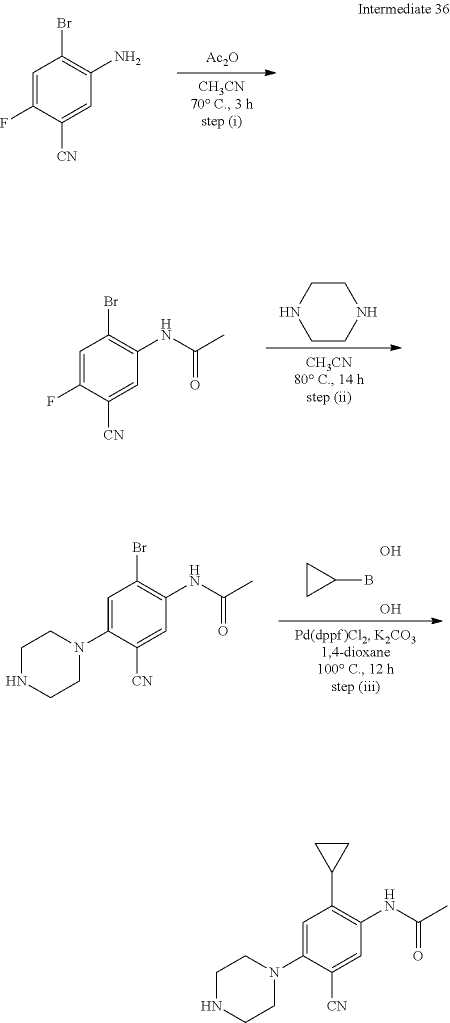

### Step (i): N-(2-Bromo-5-cyano-4-fluorophenyl)acetamide

To a stirred solution of 5-amino-4-bromo-2-fluorobenzonitrile (5 g, 23.25 mmol) in acetonitrile (50 mL) at 0° C. was added acetic anhydride (4.7 mL, 46.51 mmol). Upon completion of the addition, the reaction mixture was brought to 70° C. and was allowed to continue at 70° C. for 3 h. After completion of the reaction was confirmed by TLC, cold water was added to the reaction mixture which was then extracted with EtOAc. The combined organic layers were washed with sat. brine, dried over anhydrous sodium sulfate and evaporated under reduced pressure. The obtained crude residue was purified by column chromatography over silica gel (10-15% EtOAc in hexane) to afford the title compound as a colorless gum (4.7 g, 79%).

### Step (ii): N-(2-Bromo-5-cyano-4-(piperazin-1-yl)phenyl)acetamide

After addition of piperazine (3.47 g, 40.43 mmol) to a stirred solution of N-(2-bromo-5-cyano-4-(piperazin-1-yl)phenyl)acetamide (4.6 g, 10.11 mmol) in acetonitrile (50 mL), the reaction was continued at 80° C. for 14 h. After the completion of the reaction was confirmed by TLC, the reaction mixture was evaporated to dryness. Next, the crude residue was purified by column chromatography over silica gel eluting with 24% of MeOH in CH₂Cl₂ to afford N-(2-bromo-5-cyano-4-(piperazin-1-yl)phenyl)acetamide as a gummy liquid (4.2 g, 73%).

### Step (iii): N-(5-cyano-2-cyclopropyl-4-(piperazin-1-yl)phenyl)acetamide

To a stirred solution of N-(2-bromo-5-cyano-4-(piperazin-1-yl)phenyl)acetamide (0.25 g, 0.774 mmol) in 1,4-dioxane (10 mL) was added cyclopropylboronic acid (80 mg, 0.928 mmol), followed by K₂CO₃ (267 mg, 1.93 mmol) and the resultant mixture was bubbled with argon for 20 min. Then Pd(dppf)Cl₂ (28 mg, 0.039 mmol) was added after which the reaction was heated to 100° C. for 12 h. After the completion of reaction was confirmed by TLC and LC-MS, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by additional column chromatography over silica gel (20-25% EtOAc in hexane) yielded N-(5-cyano-2-cyclopropyl-4-(piperazin-1-yl)phenyl)acetamide as a gummy solid (125 mg, 57%).

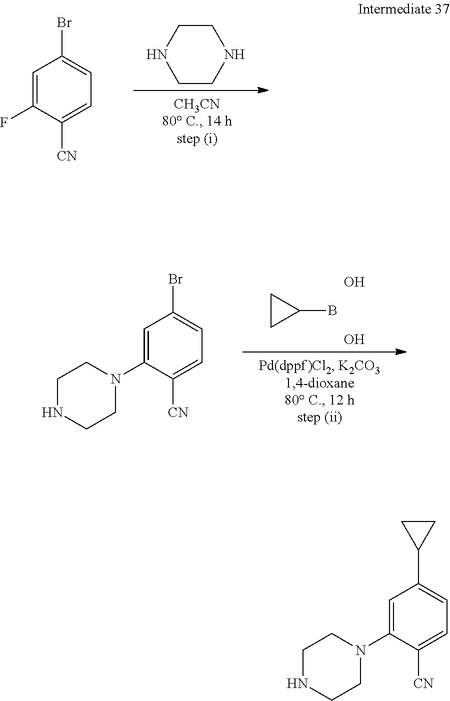

### Step (i): 4-Bromo-2-(piperazin-1-yl)benzonitrile

After addition of piperazine (645 mg, 7.5 mmol) to a stirred solution of 4-bromo-2-fluorobenzonitrile (300 mg, 1.5 mmol) in acetonitrile (10 mL), the reaction was continued at 80° C. for 14 h. After completion of the reaction was confirmed by TLC, the reaction mixture was evaporated to dryness, water was added and the resulting solution was extracted with EtOAc. The combined organic layers were washed with sat. brine, dried over anhydrous sodium sulfate and evaporated under reduced pressure. The obtained crude residue was purified by column chromatography over silica gel (2-4% MeOH in CH₂Cl₂), yielding 4-bromo-2-(piperazin-1-yl)benzonitrile as gummy liquid (0.31 g, 77%).

### Step (ii): 2-(Piperazin-1-yl)-4-cyclopropylbenzonitrile

To a stirred solution of 4-bromo-2-(piperazin-1-yl)benzonitrile (0.31 g, 1.12 mmol) in 1,4-dioxane (15 mL) was added cyclopropylboronic acid (0.117 g, 1.35 mmol), followed by K₂CO₃ (0.387 g, 2.80 mmol) and the resulting mixture was bubbled with argon for 20 min.

Then Pd(dppf)Cl₂ (0.041 g, 0.056 mmol) was added after which the reaction was heated to 80° C. for 12 h. After the completion of reaction was confirmed by TLC and LC-MS, the reaction mixture was evaporated in vacuo to remove the volatiles and the residue was re-dissolved with ethyl acetate and washed with water and sat. brine. Subsequent drying over anhydrous sodium sulfate and solvent evaporation under reduced pressure afforded 2-(piperazin-1-yl)-4-cyclopropylbenzonitrile, which was further purified via column chromatography over silica gel (10-20% EtOAc in hexane) to obtain the envisaged substituted benzonitrile as a pale-yellow gum (0.21 g, 82%).

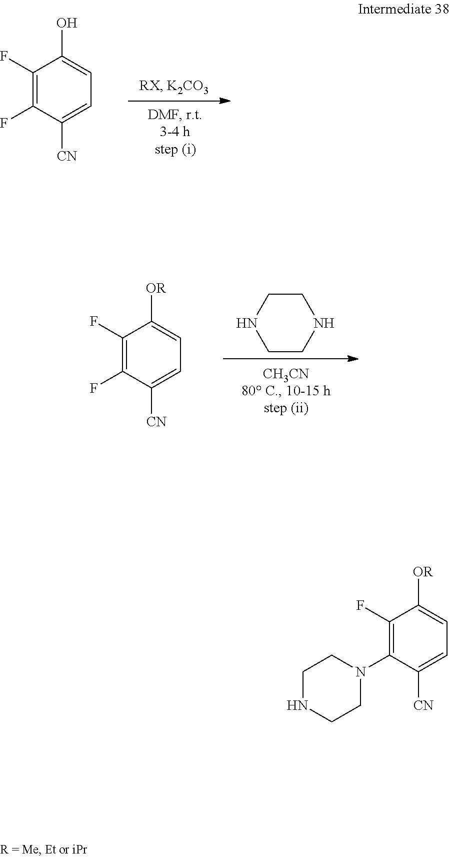

### Step (i): 4-Alkoxy-2,3-difluorobenzonitrile

To a stirred solution of 2,3-difluoro-4-hydroxybenzonitrile (1 equiv., 1.94 mmol) in DMF (10 mL) was added K₂CO₃ (2 equiv., 3.87 mmol) at 0° C., after which the solution was allowed to warm up to room temperature. After stirring at r.t. for 10 minutes, an alkyl halide of interest (1.5 equiv., 2.90 mmol) was added, after which the reaction mixture was left stirring at room temperature. After completion of the reaction was confirmed by TLC, the reaction mixture was diluted with water and extracted with ethyl acetate. The combined organic layers were washed with water, sat. brine, dried over anhydrous sodium sulfate and evaporated under reduced pressure to afford a crude residue. The obtained residual compound was purified by column chromatography over silica gel, eluting with EtOAc in hexane, affording the 4-alkoxy-2,3-difluorobenzonitrile of interest.

### Step (ii): 4-Alkoxy-3-fluoro-2-(piperazin-1-yl)benzonitrile

To a stirred solution of 4-alkoxy-2,3-difluorobenzonitrile (1 equiv., 1.27 mmol) in acetonitrile (10 mL) was added piperazine (5 equiv., 0.545 g, 6.35 mmol), after which the resulting solution was stirred at 80° C. for 10-15 h. After completion of the reaction was confirmed by TLC, the reaction mixture was evaporated to dryness, water was added and the resulting solution was then extracted with EtOAc. The combined organic layers were washed with sat. brine, dried over anhydrous sodium sulfate and concentrated in vacuo. Subsequent purification via column chromatography (SiO₂, eluting with MeOH in CH₂Cl₂), afforded the targeted 4-alkoxy-3-fluoro-2-(piperazin-1-yl)benzonitrile.

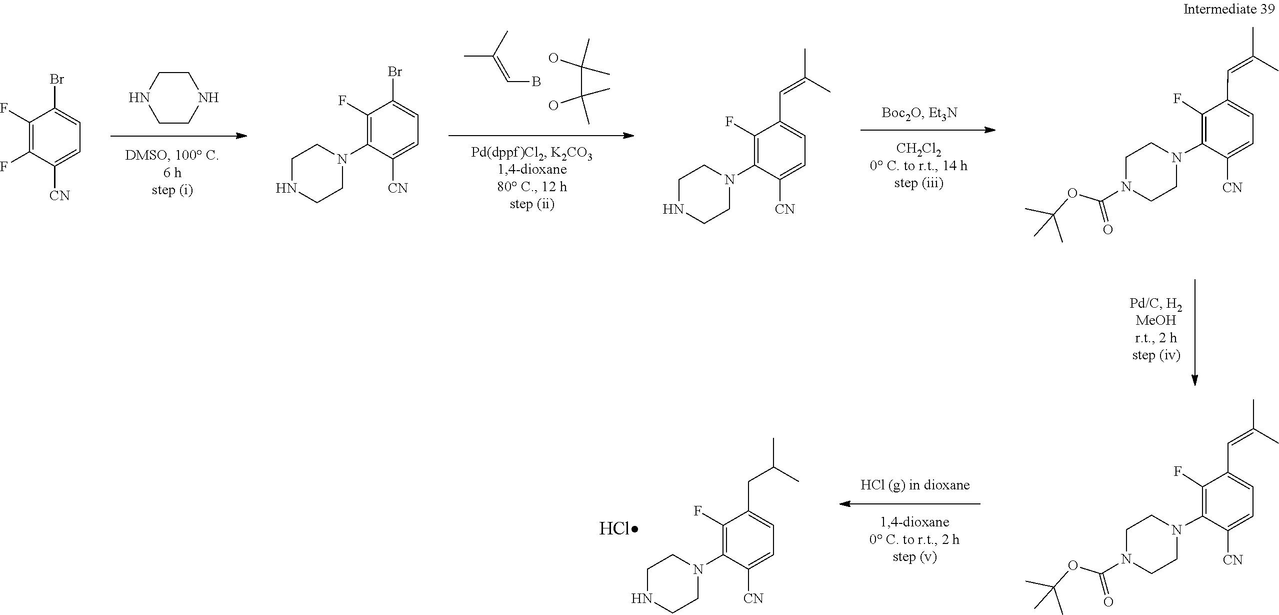

### Step (i): 4-Bromo-3-fluoro-2-(piperazin-1-yl)benzonitrile

After addition of piperazine (0.042 g, 0.481 mmol) to a stirred solution of 4-bromo-2,3-difluorobenzonitrile (0.070 g, 0.321 mmol) in DMSO (10 mL), the reaction was continued at 100° C. for 6 h. After the completion of the reaction was confirmed by TLC, cold water was added to the reaction mixture which was then extracted with ethyl acetate. The combined organic layers were washed with sat. brine, dried over anhydrous sodium sulfate and concentrated in vacuo, yielding 4-bromo-3-fluoro-2-(piperazin-1-yl)benzonitrile as a white solid (0.08 g, 87%).

### Step (ii): 3-Fluoro-4-(2-methylprop-1-en-1-yl)-2-(piperazin-1-yl)benzonitrile

To a stirred solution of 4-bromo-3-fluoro-2-(piperazin-1-yl)benzonitrile (0.08 g, 0.282 mmol) in 1,4-dioxane (20 mL) was added 4,4,5,5-tetramethyl-2-(2-methylprop-1-en-1-yl)-1,3,2-dioxaborolane (0.076 g, 0.422 mmol), followed by K₂CO₃ (0.096 g, 0.704 mmol) and the resulting mixture was bubbled with argon for 20 min. Then Pd(dppf)Cl₂ (0.010 g, 0.014 mmol) was added after which the reaction was heated to 80° C. for 12 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure afforded 3-fluoro-4-(2-methylprop-1-en-1-yl)-2-(piperazin-1-yl)benzonitrile which was taken to the next step without additional purification.

### Step (iii): tert-Butyl 4-(6-cyano-2-fluoro-3-(2-methylprop-1-en-1-yl)phenyl)piperazine-1-carboxylate

To an ice-cold solution of 3-fluoro-4-(2-methylprop-1-en-1-yl)-2-(piperazin-1-yl)benzonitrile in CH₂Cl₂ (25 mL) was added Et₃N (0.10 mL, 0.705 mmol). The solution was stirred at 0° C. for 10 minutes, after which Boc-anhydride (0.092 g, 0.423 mmol) was added and the reaction was allowed to continue at room temperature for an additional 14 hours. After completion of the reaction was confirmed by TLC, cold water was added to the reaction mixture which was then extracted with CH₂Cl₂. The combined organic layers were washed with sat. brine, dried over anhydrous sodium sulfate and evaporated under reduced pressure. The crude obtained was purified by column chromatography over silica gel (15-20% EtOAc in hexane) to afford tert-butyl 4-(6-cyano-2-fluoro-3-(2-methylprop-1-en-1-yl)phenyl)piperazine-1-carboxylate as a colorless gum (0.072 g, 71% over 2 steps).

### Step (iv): tert-Butyl 4-(6-cyano-2-fluoro-3-isobutylphenyl)piperazine-1-carboxylate

A stirred solution of tert-butyl 4-(6-cyano-2-fluoro-3-(2-methylprop-1-en-1-yl)phenyl)piperazine-1-carboxylate (0.072 g, 0.20 mmol) in MeOH (20 mL) was hydrogenated over 10% Pd/C (0.020 g) under 5 Kg/cm² H₂ pressure using a Parr hydrogenator for 2 h at ambient temperature. After confirming the completion of reaction by LC-MS, the reaction mixture was filtered through a Celite® bed and was concentrated in vacuo, yielding the desired compound as a colorless gum (0.060 g, 78%).

### Step (v): 3-Fluoro-4-isobutyl-2-(piperazin-1-yl)benzonitrile hydrochloride

tert-Butyl 4-(6-cyano-2-fluoro-3-isobutylphenyl)piperazine-1-carboxylate (0.060 g, 0.166 mmol) was dissolved in 4M HCl in 1,4-dioxane (5 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford 3-fluoro-4-isobutyl-2-(piperazin-1-yl)benzonitrile hydrochloride as an off-white solid (0.038 g, 53%).

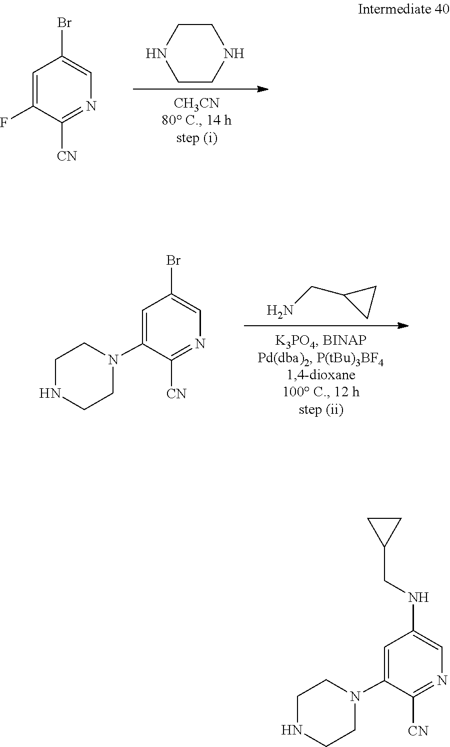 [SMILES: N#Cc1ncc(Br)cc1F ; C1CNCCN1 ; N#Cc1ncc(Br)cc1N1CCNCC1 ; NCC1CC1 ; N#Cc1ncc(NCC2CC2)cc1N1CCNCC1]

### Step (i): 5-Bromo-3-piperazin-1-yl-pyridine-2-carbonitrile

To a stirred solution of 5-bromo-3-fluoro-pyridine-2-carbonitrile (1.00 g, 4.97 mmol) in acetonitrile (25 mL) was added piperazine (2.14 g, 24.87 mmol) and the resulting mixture was heated to 80° C. for 14 h. After the completion of the reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (2-3% MeOH in CH₂Cl₂), afforded 5-bromo-3-piperazin-1-yl-pyridine-2-carbonitrile as a gummy liquid (0.81 g, 62%).

### Step (ii): 5-(Cyclopropylmethylamino)-3-piperazin-1-yl-pyridine-2-carbonitrile

To a stirred solution of 5-bromo-3-piperazin-1-yl-pyridine-2-carbonitrile (0.25 g, 0.936 mmol) in 1,4-dioxane (10 mL) was added cyclopropylmethanamine (0.079 g, 1.123 mmol), followed by K₃PO₄ (0.496 g, 2.34 mmol) and BINAP (0.023 g, 0.0374 mmol). The resultant mixture was bubbled with argon for 20 min. Then P(tBu)₃·BF₄ (0.011 g, 0.0374 mmol) followed by Pd(dba)₂ (0.027 g, 0.0468 mmol) were added after which the reaction was heated to 100° C. for 12 h. After the completion of the reaction was confirmed by TLC, the reaction mixture was evaporated in vacuo to remove the volatiles and the residue was re-dissolved with ethyl acetate and washed with water and sat. brine. Drying over anhydrous sodium sulfate and concentration in vacuo, followed by column chromatography over silica gel (25-30% EtOAc in hexane), afforded the desired compound as a pale-yellow gum (0.22 g, 91%).

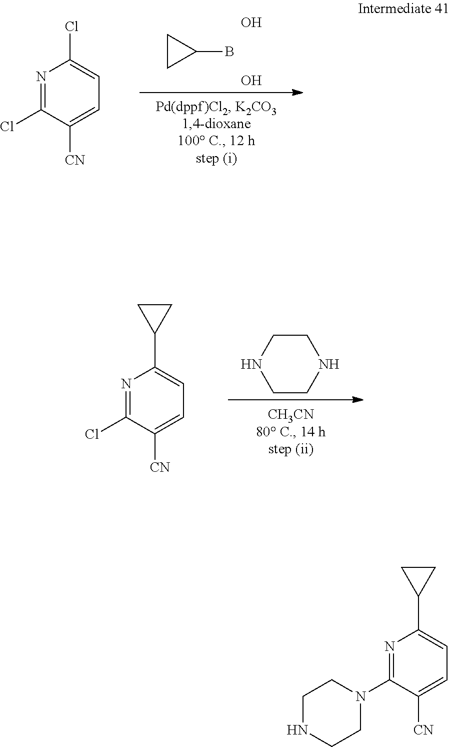

### Step (i): 2-Chloro-6-cyclopropyl-pyridine-3-carbonitrile

To a stirred solution of 2,6-dichloropyridine-3-carbonitrile (400 mg, 2.31 mmol) in 1,4-dioxane (20 mL) was added cyclopropylboronic acid (218 mg, 2.54 mmol), followed by K₂CO₃ (798 mg, 5.78 mmol) and the resulting mixture was bubbled with argon for 20 min. Then Pd(dppf)Cl₂ (85 mg, 0.116 mmol) was added after which the reaction was heated to 100° C. for 12 h. After the completion of the reaction was confirmed by TLC and LC-MS, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (20-30% EtOAc in hexane), afforded the desired 2-chloro-6-cyclopropyl-pyridine-3-carbonitrile as a colorless gum (252 mg, 61%).

### Step (ii): 6-Cyclopropyl-2-piperazin-1-yl-pyridine-3-carbonitrile

To a stirred solution of 2-chloro-6-cyclopropyl-pyridine-3-carbonitrile (250 mg, 1.40 mmol) in acetonitrile (10 mL) was added piperazine (603 mg, 7.00 mmol), after which the resulting solution was stirred at 80° C. for 14 h. After completion of the reaction was confirmed by TLC, the reaction mixture was evaporated to dryness, water was added and the resulting solution was extracted with EtOAc. The combined organic layers were washed with sat. brine, dried over anhydrous sodium sulfate and concentrated in vacuo. Subsequent purification via column chromatography (SiO₂, eluting with 2-3% MeOH in CH₂Cl₂), delivered 6-cyclopropyl-2-piperazin-1-yl-pyridine-3-carbonitrile as a viscous liquid (297 mg, 93%).

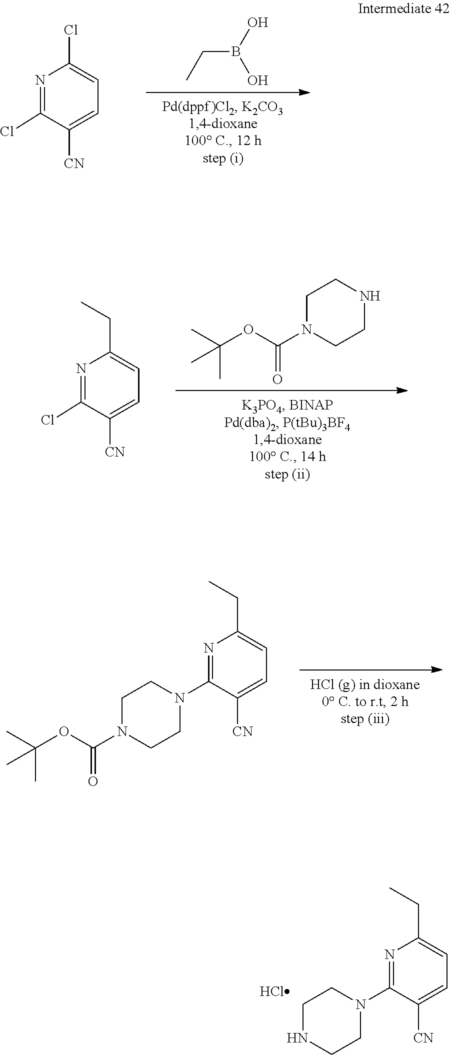

### Step (i): 2-Chloro-6-ethyl-pyridine-3-carbonitrile

To a stirred solution of 2,6-dichloropyridine-3-carbonitrile (0.25 g, 1.445 mmol) in 1,4-dioxane (10 mL) was added ethylboronic acid (0.117 g, 1.590 mmol), followed by K₂CO₃ (0.499 g, 3.613 mmol) and the resultant mixture was bubbled with argon for 20 min. Then Pd(dppf)Cl₂ (0.053 g, 0.0723 mmol) was added after which the reaction was heated to 100° C. for 12 h. After confirming complete conversion of the reaction by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (20-30% EtOAc in hexane), afforded 2-chloro-6-ethyl-pyridine-3-carbonitrile as a colorless liquid (0.195 g, 80%).

### Step (ii): tert-Butyl 4-(3-cyano-6-ethyl-2-pyridyl)piperazine-1-carboxylate

To a stirred solution of 2-chloro-6-ethyl-pyridine-3-carbonitrile (0.195 g, 1.170 mmol) in 1,4-dioxane (10 mL) was added tert-butyl piperazine-1-carboxylate (0.262 g, 1.40 mmol), followed by K₃PO₄ (0.404 g, 2.93 mmol) and BINAP (0.029 g, 0.0468 mmol). The resultant mixture was bubbled with argon for 20 min. Then P(tBu)₃·BF₄ (0.014 g, 0.0468 mmol), followed by Pd(dba)₂ (0.034 g, 0.058 mmol), were added after which the reaction was heated to 100° C. for 14 h. After the completion of the reaction was confirmed by TLC, the reaction mixture was evaporated in vacuo to remove the volatiles and the residue was re-dissolved with ethyl acetate and washed with water and sat. brine. Drying over anhydrous sodium sulfate and concentration in vacuo, followed by column chromatography over silica gel (25-30% EtOAc in hexane), afforded the desired compound as a pale-yellow liquid (0.125 g, 34%).

### Step (iii): 6-Ethyl-2-piperazin-1-yl-pyridine-3-carbonitrile hydrochloride

tert-Butyl 4-(3-cyano-6-ethyl-2-pyridyl)piperazine-1-carboxylate (0.12 g, 0.379 mmol) was dissolved in 4M HCl in 1,4-dioxane (10 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford 6-ethyl-2-piperazin-1-yl-pyridine-3-carbonitrile hydrochloride as an off-white solid (0.08 g, 84%).

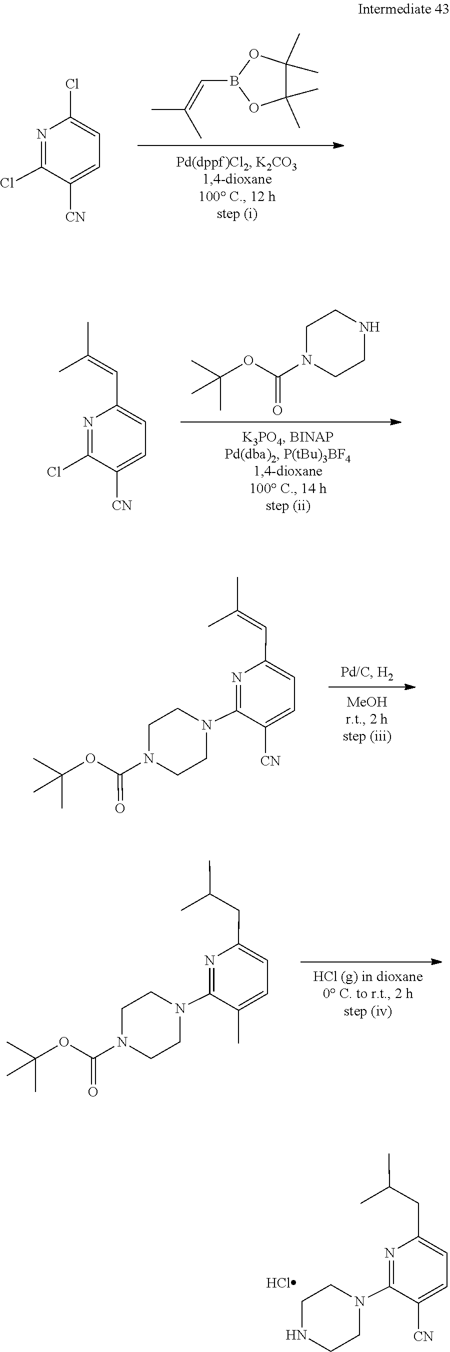

### Step (i): 2-Chloro-6-(2-methylprop-1-enyl)pyridine-3-carbonitrile

To a stirred solution of 2,6-dichloropyridine-3-carbonitrile (0.50 g, 2.89 mmol) in 1,4-dioxane (15 mL) was added 4,4,5,5-tetramethyl-2-(2-methylprop-1-en-1-yl)-1,3,2-dioxaborolane (0.578 g, 3.18 mmol), followed by K₂CO₃ (0.998 g, 7.225 mmol) and the resultant mixture was bubbled with argon for 20 min. Then Pd(dppf)Cl₂ (0.106 g, 0.145 mmol) was added after which the reaction was heated to 100° C. for 12 h. After the completion of the reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (20-30% EtOAc in hexane) afforded 2-chloro-6-(2-methylprop-1-enyl)pyridine-3-carbonitrile as a colorless gum (0.22 g, 40%).

### Step (ii): tert-Butyl 4-[3-cyano-6-(2-methylprop-1-enyl)-2-pyridyl]piperazine-1-carboxylate

To a stirred solution of 2-chloro-6-(2-methylprop-1-enyl)pyridine-3-carbonitrile (0.220 g, 1.14 mmol) in 1,4-dioxane (10 mL) was added tert-butyl piperazine-1-carboxylate (0.319 g, 1.71 mmol), followed by K₃PO₄ (0.606 g, 2.86 mmol) and BINAP (0.071 g, 0.114 mmol). The resultant mixture was bubbled with argon for 20 min. Then P(tBu)₃·BF₄ (0.033 g, 0.114 mmol) followed by Pd(dba)₂ (0.033 g, 0.057 mmol) were added after which the reaction was heated to 100° C. for 14 h. After the completion of reaction was confirmed by TLC, the reaction mixture was evaporated in vacuo to remove the volatiles and the residue was re-dissolved with ethyl acetate and washed with water and sat. brine. Drying over anhydrous sodium sulfate and concentration in vacuo, followed by column chromatography over silica gel (20-30% EtOAc in hexane), afforded the desired compound as a colorless gum (0.15 g, 38%).

### Step (iii): tert-Butyl 4-(3-cyano-6-isobutyl-2-pyridyl)piperazine-1-carboxylate

A stirred solution of tert-butyl 4-[3-cyano-6-(2-methylprop-1-enyl)-2-pyridyl]piperazine-1-carboxylate (0.15 g, 0.434 mmol) in MeOH (10 mL) was hydrogenated over 10% Pd/C (0.04 g) under 5 Kg/cm² H₂ pressure using a Parr hydrogenator for 2 h at ambient temperature. After confirming the completion of reaction by LC-MS, the reaction mixture was filtered through a Celite® bed and was concentrated in vacuo, yielding the envisaged hydrogenated compound as a colorless viscous liquid (0.12 g, 80%).

### Step (iv): 6-Isobutyl-2-piperazin-1-yl-pyridine-3-carbonitrile hydrochloride

tert-Butyl 4-(3-cyano-6-isobutyl-2-pyridyl)piperazine-1-carboxylate (0.12 g, 0.348 mmol) was dissolved in 4M HCl in 1,4-dioxane (10 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford 6-isobutyl-2-piperazin-1-yl-pyridine-3-carbonitrile hydrochloride as an off-white solid (0.087 g, 89%).

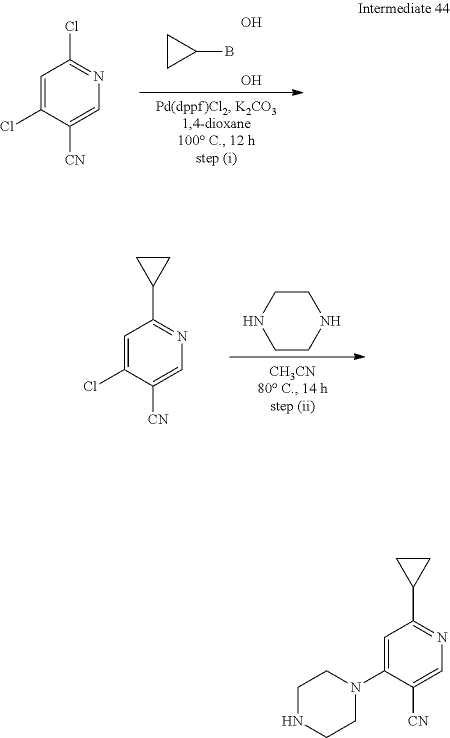

### Step (i): 4-Chloro-6-cyclopropyl-pyridine-3-carbonitrile

To a stirred solution of 4,6-dichloropyridine-3-carbonitrile (0.50 g, 2.89 mmol) in 1,4-dioxane (20 mL) was added cyclopropylboronic acid (0.273 g, 3.18 mmol), followed by K₂CO₃ (0.998 g, 7.225 mmol) and the resulting mixture was bubbled with argon for 20 min. Then Pd(dppf)Cl₂ (0.106 g, 0.145 mmol) was added after which the reaction was heated to 100° C. for 12 h. After the completion of the reaction was confirmed by TLC and LC-MS, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (20-30% EtOAc in hexane), yielded the desired 4-chloro-6-cyclopropyl-pyridine-3-carbonitrile as a colorless solid (0.175 g, 34%).

### Step (ii): 6-Cyclopropyl-4-piperazin-1-yl-pyridine-3-carbonitrile

To a stirred solution of 4-chloro-6-cyclopropyl-pyridine-3-carbonitrile (0.17 g, 0.952 mmol) in acetonitrile (10 mL) was added piperazine (0.410 g, 4.76 mmol), after which the resulting solution was stirred at 80° C. for 14 h. After completion of the reaction was confirmed by TLC, the reaction mixture was evaporated to dryness, diluted with water and extracted with EtOAc. The combined organic layers were washed with sat. brine, dried over anhydrous sodium sulfate and concentrated in vacuo. Subsequent purification via column chromatography (SiO₂, eluting with 2-4% MeOH in CH₂Cl₂), delivered the targeted 6-cyclopropyl-4-piperazin-1-yl-pyridine-3-carbonitrile as a gummy liquid (0.18 g, 82%).

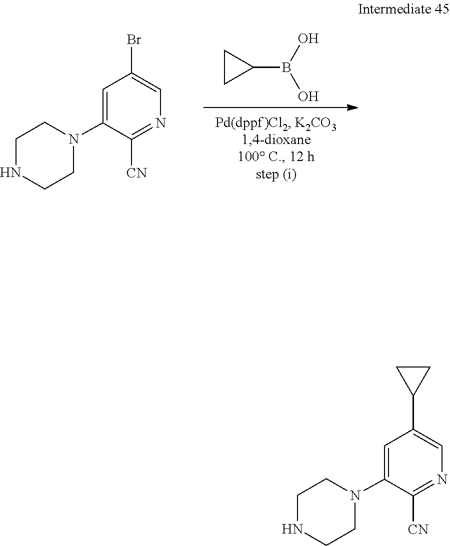 [SMILES: N#Cc1ncc(Br)cc1N1CCNCC1 ; OB(O)C1CC1 ; N#Cc1ncc(C2CC2)cc1N1CCNCC1]

### Step (i): 5-Cyclopropyl-3-piperazin-1-yl-pyridine-2-carbonitrile

To a stirred solution of 5-bromo-3-piperazin-1-yl-pyridine-2-carbonitrile (300 mg, 1.12 mmol) in 1,4-dioxane (20 mL) was added cyclopropylboronic acid (116 mg, 1.35 mmol), followed by K₂CO₃ (388 mg, 2.81 mmol) and the resulting mixture was bubbled with argon for 20 min. Then Pd(dppf)Cl₂ (41 mg, 0.056 mmol) was added after which the reaction was heated to 100° C. for 12 h. After the completion of the reaction was confirmed by TLC and LC-MS, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (2-3% MeOH in CH₂Cl₂), afforded the desired 5-cyclopropyl-3-piperazin-1-yl-pyridine-2-carbonitrile as a colorless gum (155 mg, 60%).

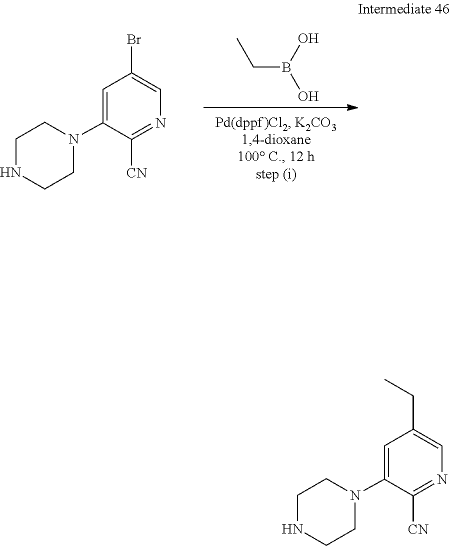

### Step (i): 5-Ethyl-3-piperazin-1-yl-pyridine-2-carbonitrile

To a stirred solution of 5-bromo-3-piperazin-1-yl-pyridine-2-carbonitrile (350 mg, 1.31 mmol) in 1,4-dioxane (20 mL) was added ethylboronic acid (116 mg, 1.57 mmol), followed by K₂CO₃ (453 mg, 3.28 mmol) and the resulting mixture was bubbled with argon for 20 min. Then Pd(dppf)Cl₂ (48 mg, 0.066 mmol) was added after which the reaction was heated to 100° C. for 12 h. After the completion of reaction was confirmed by TLC and LC-MS, the reaction mixture was concentrated under reduced pressure to remove the volatiles and the residue was re-dissolved with ethyl acetate and washed with water and sat. brine. Subsequent drying over anhydrous sodium sulfate and evaporation under reduced pressure, followed by column chromatography over silica gel (2-3% MeOH in CH₂Cl₂), delivered 5-ethyl-3-piperazin-1-yl-pyridine-2-carbonitrile as a colorless gum (250 mg, 88%).

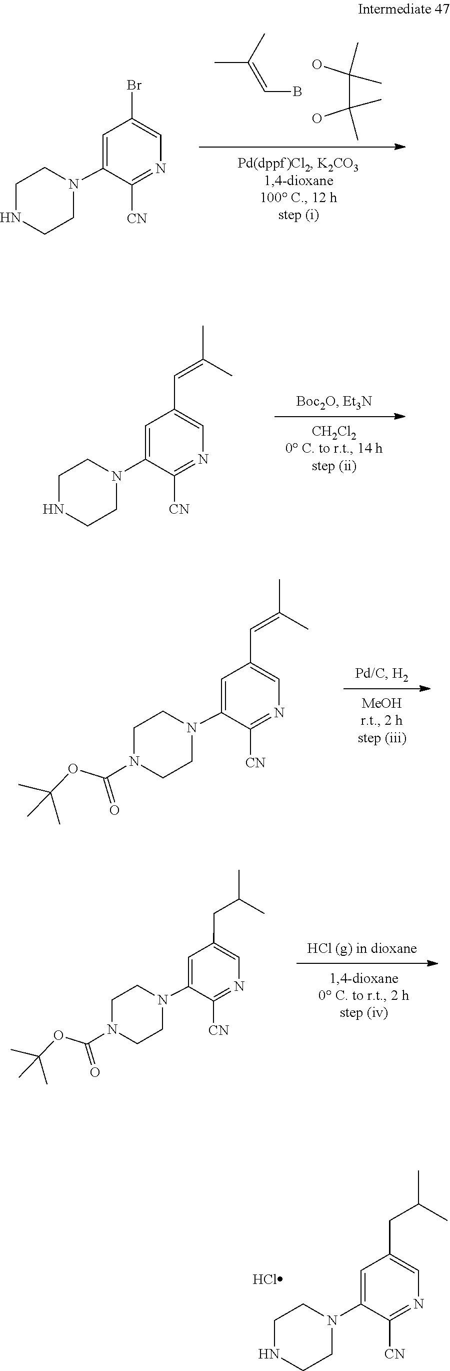

### Step (i): 5-(2-Methylprop-1-enyl)-3-piperazin-1-yl-pyridine-2-carbonitrile

To a stirred solution of 5-bromo-3-piperazin-1-yl-pyridine-2-carbonitrile (0.30 g, 1.12 mmol) in 1,4-dioxane (15 mL) was added 4,4,5,5-tetramethyl-2-(2-methylprop-1-en-1-yl)-1,3,2-dioxaborolane (245 mg, 1.35 mmol), followed by K₂CO₃ (388 mg, 2.81 mmol) and the resultant mixture was bubbled with argon for 20 min. Then Pd(dppf)Cl₂ (41 mg, 0.056 mmol) was added after which the reaction was heated to 100° C. for 12 h. After the completion of the reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure afforded 5-(2-methylprop-1-enyl)-3-piperazin-1-yl-pyridine-2-carbonitrile which was used as such in the next step without additional purification.

### Step (ii): tert-Butyl 4-[2-cyano-5-(2-methylprop-1-enyl)-3-pyridyl]piperazine-1-carboxylate

To an ice-cold solution of 5-(2-methylprop-1-enyl)-3-piperazin-1-yl-pyridine-2-carbonitrile in CH₂Cl₂ (10 mL) was added Et₃N (0.392 mL, 2.81 mmol). The solution was stirred at 0° C. for 10 minutes, after which Boc-anhydride (0.368 g, 1.68 mmol) was added and the reaction was allowed to continue at room temperature for an additional 14 hours. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The obtained crude was purified by column chromatography over silica gel (20-30% EtOAc in hexane) to afford tert-butyl 4-[2-cyano-5-(2-methylprop-1-enyl)-3-pyridyl]piperazine-1-carboxylate as a colorless gum (0.15 g, 39% over 2 steps).

### Step (iii): tert-Butyl 4-(2-cyano-5-isobutyl-3-pyridyl)piperazine-1-carboxylate

A stirred solution of tert-butyl 4-[2-cyano-5-(2-methylprop-1-enyl)-3-pyridyl]piperazine-1-carboxylate (0.15 g, 0.438 mmol) in MeOH (10 mL) was hydrogenated over 10% Pd/C (0.04 g) under 5 Kg/cm² H₂ pressure using a Parr hydrogenator for 2 h at ambient temperature. After confirming the completion of reaction by LC-MS, the reaction mixture was filtered through a Celite® bed and was concentrated in vacuo, yielding the desired compound as a colorless gum (0.12 g, 83%).

### Step (iv): 5-Isobutyl-3-piperazin-1-yl-pyridine-2-carbonitrile hydrochloride

tert-Butyl 4-(2-cyano-5-isobutyl-3-pyridyl)piperazine-1-carboxylate (0.12 g, 0.348 mmol) was dissolved in 4M HCl in 1,4-dioxane (10 mL) at 0° C., after which the solution was slowly warmed up to room temperature and continued stirring at r.t. for 2 h. After the completion of the reaction was confirmed by TLC, the reaction mixture was evaporated to dryness under reduced pressure. The crude residue was further triturated with diethyl ether to afford the targeted hydrochloride salt as an off-white solid (0.088 g, 90%).

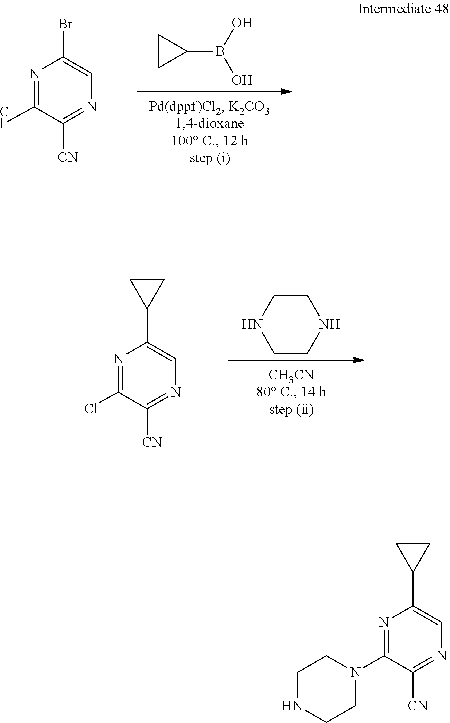 [SMILES: N#Cc1ncc(Br)nc1Cl ; OB(O)C1CC1 ; N#Cc1ncc(C2CC2)nc1Cl ; C1CNCCN1 ; N#Cc1ncc(C2CC2)nc1N1CCNCC1]

### Step (i): 3-Chloro-5-cyclopropyl-pyrazine-2-carbonitrile

To a stirred solution of 3,5-dichloropyrazine-2-carbonitrile (0.50 g, 2.87 mmol) in 1,4-dioxane (20 mL) was added cyclopropylboronic acid (296 mg, 3.45 mmol), followed by K₂CO₃ (993 mg, 7.18 mmol) and the resulting mixture was bubbled with argon for 20 min. Then Pd(dppf)Cl₂ (105 mg, 0.144 mmol) was added after which the reaction was heated to 100° C. for 12 h. After the completion of the reaction was confirmed by TLC and LC-MS, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (2-3% MeOH in CH₂Cl₂), afforded the desired 3-chloro-5-cyclopropyl-pyrazine-2-carbonitrile as a colorless gum (310 mg, 60%).

### Step (ii): 5-Cyclopropyl-3-piperazin-1-yl-pyrazine-2-carbonitrile

To a stirred solution of 3-chloro-5-cyclopropyl-pyrazine-2-carbonitrile (0.30 g, 1.67 mmol) in acetonitrile (15 mL) was added piperazine (0.72 g, 8.35 mmol), after which the resulting solution was stirred at 80° C. for 14 h. After completion of the reaction was confirmed by TLC, the reaction mixture was evaporated to dryness, water was added and the resulting solution was then extracted with EtOAc. The combined organic layers were washed with sat. brine, dried over anhydrous sodium sulfate and concentrated in vacuo. Subsequent purification via column chromatography (SiO₂, eluting with 2-4% MeOH in CH₂Cl₂), delivered the targeted 5-cyclopropyl-3-piperazin-1-yl-pyrazine-2-carbonitrile as a gummy liquid (0.30 g, 79%).

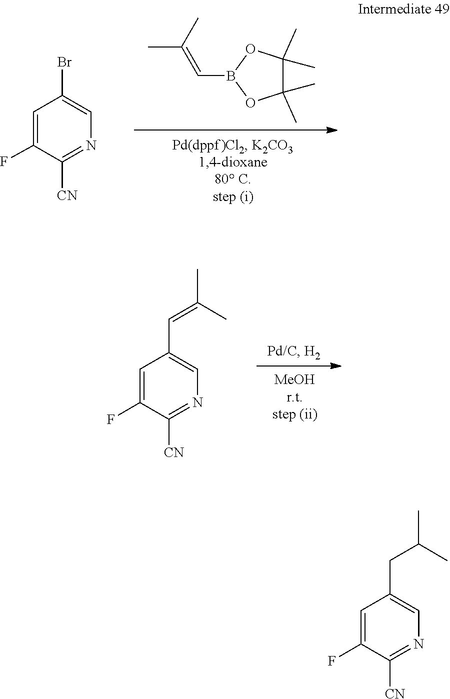

### Step (i): 3-Fluoro-5-(2-methylprop-1-enyl)pyridine-2-carbonitrile

To a stirred solution of 5-bromo-3-fluoro-pyridine-2-carbonitrile (500 mg, 2.49 mmol) in 1,4-dioxane (10 mL) was added 4,4,5,5-tetramethyl-2-(2-methylprop-1-en-1-yl)-1,3,2-dioxaborolane (544 mg, 2.99 mmol), followed by K₂CO₃ (860 mg, 6.22 mmol) and the resultant mixture was bubbled with argon for 20 min. Then Pd(dppf)Cl₂ (182 mg, 0.249 mmol) was added after which the reaction was heated to 80° C. for 6 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (20-25% EtOAc in hexane) afforded 3-fluoro-5-(2-methylprop-1-enyl)pyridine-2-carbonitrile as a colorless gum (385 mg, 88%).

### Step (ii): 3-Fluoro-5-isobutyl-pyridine-2-carbonitrile

A stirred solution of 3-fluoro-5-(2-methylprop-1-enyl)pyridine-2-carbonitrile (300 mg, 1.70 mmol) in MeOH (10 mL) was hydrogenated over 10% Pd/C (0.050 g) under 5 Kg/cm² H₂ pressure using a Parr hydrogenator for 3 h at ambient temperature. After confirming the completion of reaction by LC-MS, the reaction mixture was filtered through a Celite® bed and was concentrated in vacuo, yielding the envisaged hydrogenated compound as a viscous liquid (270 mg, 89%).

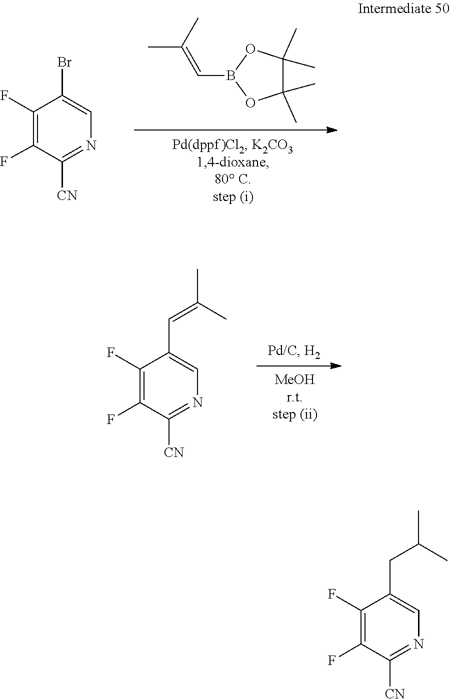

### Step (i): 2,3-Difluoro-4-(2-methylprop-1-enyl)benzonitrile

To a stirred solution of 4-bromo-2,3-difluoro-benzonitrile (200 mg, 0.917 mmol) in 1,4-dioxane (5 mL) was added 4,4,5,5-tetramethyl-2-(2-methylprop-1-en-1-yl)-1,3,2-dioxaborolane (200 mg, 1.10 mmol), followed by K₂CO₃ (317 mg, 2.29 mmol) and the resultant mixture was bubbled with argon for 20 min. Then Pd(dppf)Cl₂ (67 mg, 0.092 mmol) was added after which the reaction was heated to 80° C. for 6 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (15-20% EtOAc in hexane), afforded 2,3-difluoro-4-(2-methylprop-1-enyl)benzonitrile as a colorless gum (160 mg, 90%).

### Step (ii): 2,3-Difluoro-4-isobutyl-benzonitrile

A stirred solution of 2,3-difluoro-4-(2-methylprop-1-enyl)benzonitrile (160 mg, 0.828 mmol) in MeOH (5 mL) was hydrogenated over 10% Pd/C (0.025 g) under 5 Kg/cm² H₂ pressure using a Parr hydrogenator for 3 h at ambient temperature. After confirming the completion of reaction by LC-MS, the reaction mixture was filtered through a Celite® bed and was concentrated in vacuo. The obtained crude residue was purified by column chromatography (SiO₂, 40-45% EtOAc in hexane), yielding the envisaged hydrogenated compound as a colorless gum (121 mg, 75%).

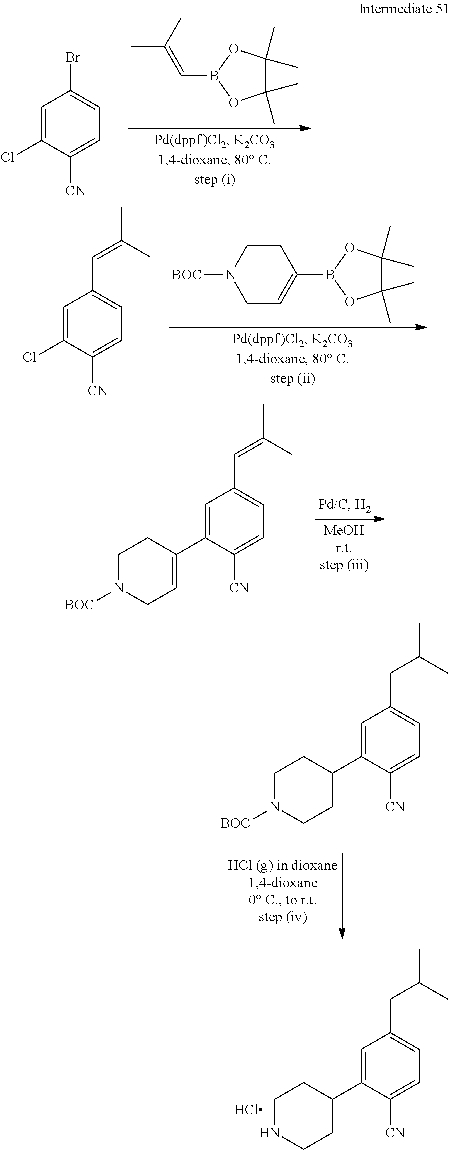

### Step (i): 2-Chloro-4-(2-methylprop-1-enyl)benzonitrile

To a stirred solution of 4-bromo-2-chloro-benzonitrile (1.00 g, 4.62 mmol) in 1,4-dioxane (20 mL) was added 4,4,5,5-tetramethyl-2-(2-methylprop-1-en-1-yl)-1,3,2-dioxaborolane (1.01 g, 5.54 mmol), followed by K₂CO₃ (1.60 g, 11.5 mmol) and the resultant mixture was bubbled with argon for 20 min. Then Pd(dppf)Cl₂ (169 mg, 0.23 mmol) was added after which the reaction was heated to 80° C. for 6 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (15-20% EtOAc in hexane), afforded 2-chloro-4-(2-methylprop-1-enyl)benzonitrile as a colorless gum (665 mg, 75%).

### Step (ii): tert-Butyl 4-[2-cyano-5-(2-methylprop-1-enyl)phenyl]-3,6-dihydro-2H-pyridine-1-carboxylate

To a stirred solution of 2-chloro-4-(2-methylprop-1-enyl)benzonitrile (300 mg, 1.57 mmol) in 1,4-dioxane (10 mL) was added tert-butyl 4-(4,4,5,5-tetramethyl-1,3,2-dioxaborolan-2-yl)-3,6-dihydro-2H-pyridine-1-carboxylate (581 mg, 1.88 mmol), followed by K₂CO₃ (541 mg, 3.91 mmol) and the resultant mixture was bubbled with argon for 20 min. Then Pd(dppf)Cl₂ (115 mg, 0.16 mmol) was added after which the reaction was heated to 100° C. for 12 h. After the completion of the reaction was confirmed by TLC, the reaction mixture was evaporated under reduced pressure to remove the volatiles and the residue was re-dissolved with ethyl acetate and washed with water and sat. brine. Subsequent drying over anhydrous sodium sulfate and evaporation in vacuo, followed by column chromatography over silica gel (25-30% EtOAc in hexane), yielded tert-butyl 4-[2-cyano-5-(2-methylprop-1-enyl)phenyl]-3,6-dihydro-2H-pyridine-1-carboxylate as a colorless gum (244 mg, 46%).

### Step (iii): tert-Butyl 4-(2-cyano-5-isobutyl-phenyl)piperidine-1-carboxylate

A stirred solution of tert-butyl 4-[2-cyano-5-(2-methylprop-1-enyl)phenyl]-3,6-dihydro-2H-pyridine-1-carboxylate (240 mg, 0.709 mmol) in MeOH (5 mL) was hydrogenated over 10% Pd/C (0.045 g) under 5 Kg/cm² H₂ pressure using a Parr hydrogenator for 3 h at ambient temperature. After confirming the completion of the reaction by LC-MS, the reaction mixture was filtered through a Celite® bed and was concentrated in vacuo. The obtained crude residue was purified by column chromatography (SiO₂, 40-45% EtOAc in hexane), delivering the desired hydrogenated compound as a colorless gum (189 mg, 78%).

### Step (iv): 4-Isobutyl-2-(4-piperidyl)benzonitrile hydrochloride

tert-Butyl 4-(2-cyano-5-isobutyl-phenyl)piperidine-1-carboxylate (180 mg, 0.526 mmol) was dissolved in 4M HCl in 1,4-dioxane (3 mL) at 0° C., after which the solution was slowly warmed up to room temperature and continued stirring at r.t. for 5 additional hours.

After the completion of the reaction was confirmed by TLC, the reaction mixture was evaporated to dryness under reduced pressure. The crude residue was further triturated with hexane to afford the targeted hydrochloride salt as an off-white solid (132 mg, 90%).

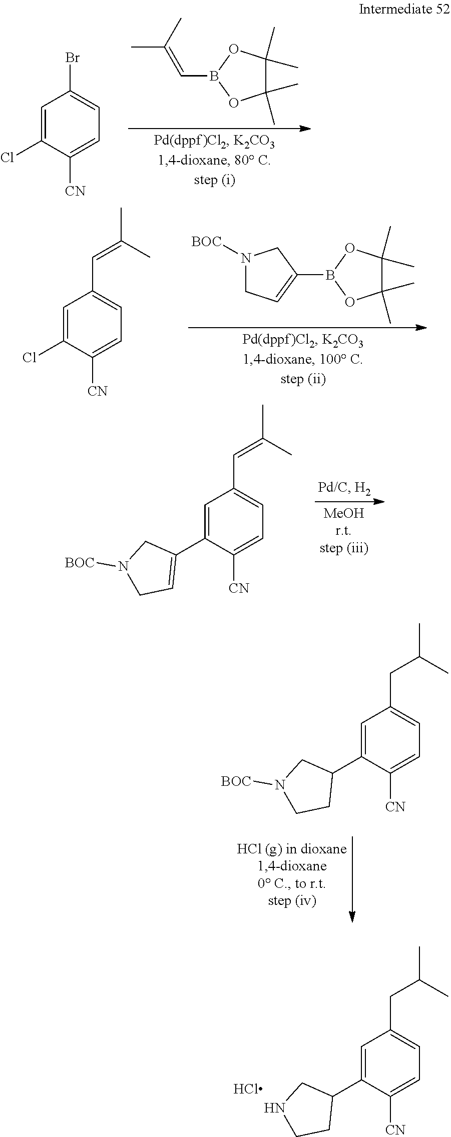

### Step (i): 2-Chloro-4-(2-methylprop-1-enyl)benzonitrile

To a stirred solution of 4-bromo-2-chloro-benzonitrile (1.00 g, 4.62 mmol) in 1,4-dioxane (20 mL) was added 4,4,5,5-tetramethyl-2-(2-methylprop-1-en-1-yl)-1,3,2-dioxaborolane (1.01 g, 5.54 mmol), followed by K₂CO₃ (1.60 g, 11.5 mmol) and the resultant mixture was bubbled with argon for 20 min. Then Pd(dppf)Cl₂ (169 mg, 0.23 mmol) was added after which the reaction was heated to 80° C. for 6 h. After the completion of the reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (15-20% EtOAc in hexane), afforded 2-chloro-4-(2-methylprop-1-enyl)benzonitrile as a colorless gum (665 mg, 75%).

### Step (ii): tert-Butyl 3-[2-cyano-5-(2-methylprop-1-enyl)phenyl]-2,5-dihydropyrrole-1-carboxylate

To a stirred solution of 2-chloro-4-(2-methylprop-1-enyl)benzonitrile (200 mg, 1.04 mmol) in 1,4-dioxane (10 mL) was added tert-butyl 3-(4,4,5,5-tetramethyl-1,3,2-dioxaborolan-2-yl)-2,5-dihydropyrrole-1-carboxylate (370 mg, 1.25 mmol), followed by K₂CO₃ (361 mg, 2.61 mmol) and the resultant mixture was bubbled with argon for 20 min. Then Pd(dppf)Cl₂ (38 mg, 0.052 mmol) was added after which the reaction was heated to 100° C. for 12 h. After the completion of the reaction was confirmed by TLC, the reaction mixture was evaporated under reduced pressure to remove the volatiles and the residue was re-dissolved with ethyl acetate and washed with water and sat. brine. Subsequent drying over anhydrous sodium sulfate and evaporation in vacuo, followed by column chromatography over silica gel (25-30% EtOAc in hexane), yielded tert-butyl 3-[2-cyano-5-(2-methylprop-1-enyl)phenyl]-2,5-dihydropyrrole-1-carboxylate as a colorless gum (190 mg, 56%).

### Step (iii): tert-Butyl 3-(2-cyano-5-isobutyl-phenyl)pyrrolidine-1-carboxylate

A stirred solution of tert-butyl 3-[2-cyano-5-(2-methylprop-1-enyl)phenyl]-2,5-dihydropyrrole-1-carboxylate (190 mg, 0.586 mmol) in MeOH (5 mL) was hydrogenated over 10% Pd/C (0.025 g) under 5 Kg/cm² H₂ pressure using a Parr hydrogenator for 3 h at ambient temperature. After confirming the completion of the reaction by LC-MS, the reaction mixture was filtered through a Celite® bed and was concentrated under reduced pressure. The obtained crude residue was purified by column chromatography (SiO₂, 40-45% EtOAc in hexane), delivering the desired hydrogenated compound as a colorless gum (143 mg, 74%).

### Step (iv): 4-Isobutyl-2-pyrrolidin-3-yl-benzonitrile hydrochloride

tert-Butyl 3-(2-cyano-5-isobutyl-phenyl)pyrrolidine-1-carboxylate (140 mg, 0.426 mmol) was dissolved in 4M HCl in 1,4-dioxane (2 mL) at 0° C., after which the solution was slowly warmed up to room temperature and continued stirring at r.t. for 5 additional hours. After the completion of the reaction was confirmed by TLC, the reaction mixture was evaporated to dryness under reduced pressure. The crude residue was further triturated with hexane to afford the targeted hydrochloride salt which was used as such in the following alkylation reaction (102 mg, 90%).

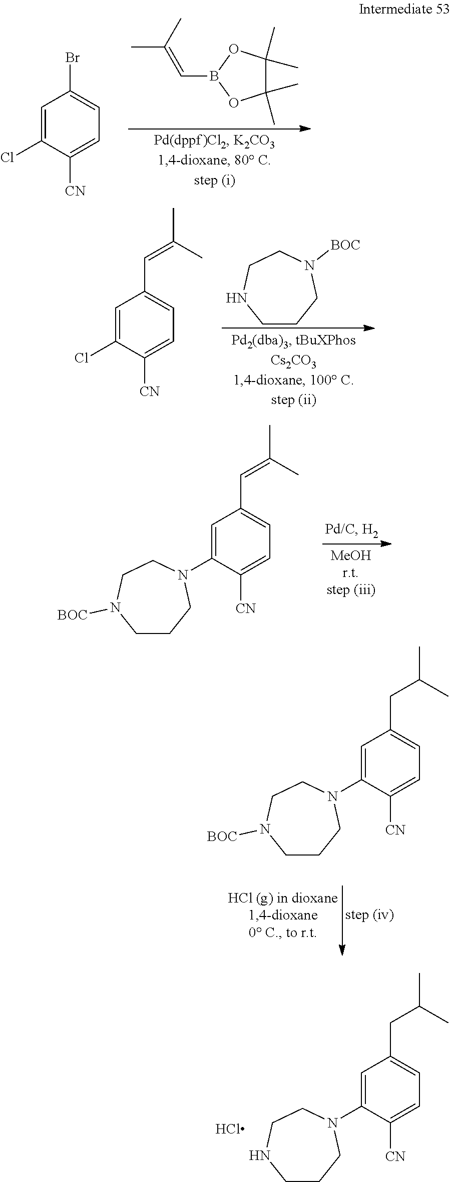

### Step (i): 2-Chloro-4-(2-methylprop-1-enyl)benzonitrile

Synthesized according to the described protocol mentioned in step (i) of intermediate 51 and intermediate 52.

### Step (ii): tert-Butyl 4-[2-cyano-5-(2-methylprop-1-enyl)phenyl]-1,4-diazepane-1-carboxylate

To a stirred solution of 2-chloro-4-(2-methylprop-1-enyl)benzonitrile (178 mg, 0.929 mmol) in 1,4-dioxane (5 mL) was added tert-butyl 1,4-diazepane-1-carboxylate (223 mg, 1.11 mmol), followed by Cs₂CO₃ (756 mg, 2.32 mmol). The resultant mixture was bubbled with argon for 20 min, after which tBuXPhos (20 mg, 0.046 mmol) and Pd₂(dba)₃ (43 mg, 0.046 mmol) were added. Upon completion of the addition, the reaction was brought to 100° C. for 14 h until full conversion of the reaction was observed by TLC. The reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (35-40% EtOAc in hexane), yielded the desired substituted nitrile as a colorless gum (135 mg, 41%).

### Step (iii): tert-Butyl 4-(2-cyano-5-isobutyl-phenyl)-1,4-diazepane-1-carboxylate

A stirred solution of tert-butyl 4-[2-cyano-5-(2-methylprop-1-enyl)phenyl]-1,4-diazepane-1-carboxylate (130 mg, 0.364 mmol) in MeOH (5 mL) was hydrogenated over 10% Pd/C (30 mg) under 5 Kg/cm² H₂ pressure using a Parr hydrogenator for 3 h at ambient temperature. After confirming the completion of the reaction by LC-MS, the reaction mixture was filtered through a Celite® bed and was concentrated under reduced pressure. The obtained crude residue was purified by column chromatography (SiO₂, 40-45% EtOAc in hexane), delivering the desired hydrogenated compound as a colorless gum (102 mg, 78%).

### Step (iv): 2-(1,4-Diazepan-1-yl)-4-isobutyl-benzonitrile hydrochloride

To a stirred solution of tert-butyl 4-(2-cyano-5-isobutyl-phenyl)-1,4-diazepane-1-carboxylate (100 mg, 0.279 mmol) in 1,4-dioxane (1 mL) was added HCl (g) in dioxane (2 mL) at 0° C., after which the solution was slowly warmed up to room temperature and continued stirring at r.t. for 5 h. After the completion of the reaction was confirmed by TLC, the reaction mixture was evaporated to dryness under reduced pressure. The crude residue was further triturated with hexane to afford the targeted hydrochloride salt as an off-white solid (70 mg, 84%).

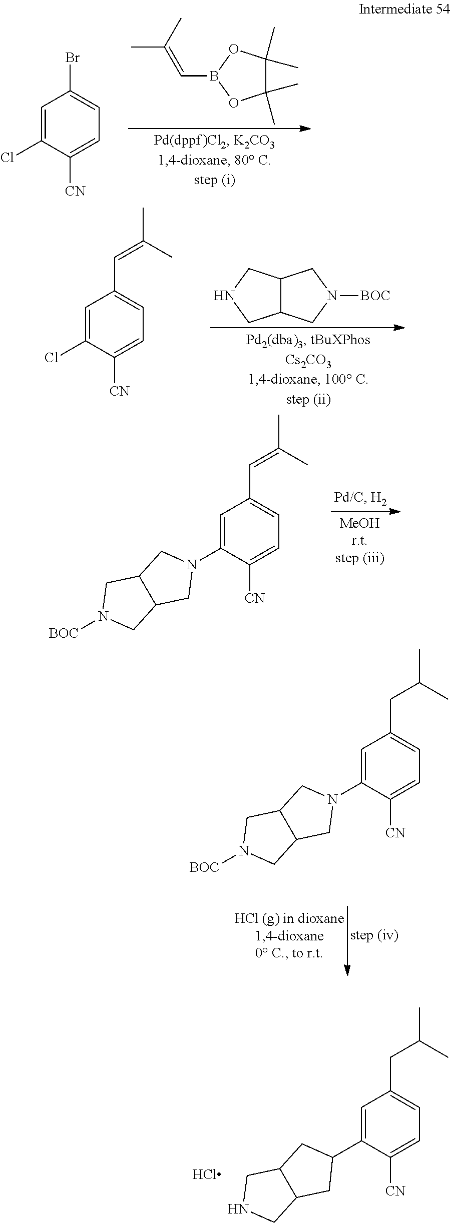

### Step (i): 2-Chloro-4-(2-methylprop-1-enyl)benzonitrile

Synthesized according to the described protocol mentioned in step (i) of intermediate 51 and intermediate 52.

### Step (ii): tert-Butyl 2-[2-cyano-5-(2-methylprop-1-enyl)phenyl]-1,3,3a,4,6,6a-hexahydropyrrolo[3,4-c]pyrrole-5-carboxylate

To a stirred solution of 2-chloro-4-(2-methylprop-1-enyl)benzonitrile (250 mg, 1.30 mmol) in 1,4-dioxane (10 mL) was added tert-butyl 2,3,3a,4,6,6a-hexahydro-1H-pyrrolo[3,4-c]pyrrole-5-carboxylate (332 mg, 1.57 mmol), followed by Cs₂CO₃ (1.06 g, 3.26 mmol). The resultant mixture was bubbled with argon for 20 min, after which tBuXPhos (28 mg, 0.065 mmol) and Pd₂(dba)₃ (60 mg, 0.065 mmol) were added. Upon completion of the addition, the reaction was brought to 100° C. for 14 h until full conversion of the reaction was observed via TLC. The reaction mixture was concentrated under reduced pressure to remove the volatiles and the residue was re-dissolved with ethyl acetate and washed with water and sat. brine. Subsequent drying over anhydrous sodium sulfate and evaporation under reduced pressure, followed by column chromatography over silica gel (35-40% EtOAc in hexane), yielded the envisaged substituted nitrile as a colorless gum (216 mg, 45%).

### Step (iii): tert-Butyl 2-(2-cyano-5-isobutyl-phenyl)-1,3,3a,4,6,6a-hexahydropyrrolo[3,4-c]pyrrole-5-carboxylate

A stirred solution of tert-butyl 2-[2-cyano-5-(2-methylprop-1-enyl)phenyl]-1,3,3a,4,6,6a-hexahydropyrrolo[3,4-c]pyrrole-5-carboxylate (198 mg, 0.539 mmol) in MeOH (5 mL) was hydrogenated over 10% Pd/C (40 mg) under 5 Kg/cm² H₂ pressure using a Parr hydrogenator for 3 h at ambient temperature. After confirming the completion of the reaction by LC-MS, the reaction mixture was filtered through a Celite® bed and was concentrated in vacuo. The obtained crude residue was purified by column chromatography (SiO₂, 40-45% EtOAc in hexane), affording the desired hydrogenated compound as a colorless gum (171 mg, 86%).

### Step (iv): 2-(2,3,3a,4,6,6a-Hexahydro-1H-pyrrolo[3,4-c]pyrrol-5-yl)-4-isobutyl-benzonitrile hydrochloride

To a stirred solution of tert-butyl 2-(2-cyano-5-isobutyl-phenyl)-1,3,3a,4,6,6a-hexahydropyrrolo[3,4-c]pyrrole-5-carboxylate (170 mg, 0.46 mmol) in 1,4-dioxane (1 mL) was added HCl (g) in dioxane (2 mL) at 0° C., after which the solution was slowly warmed up to room temperature and kept stirring at r.t. for an additional 5 hours. After the completion of the reaction was confirmed by TLC, the reaction mixture was evaporated to dryness under reduced pressure. The obtained crude hydrochloride salt was used as such in the following reaction (135 mg crude).

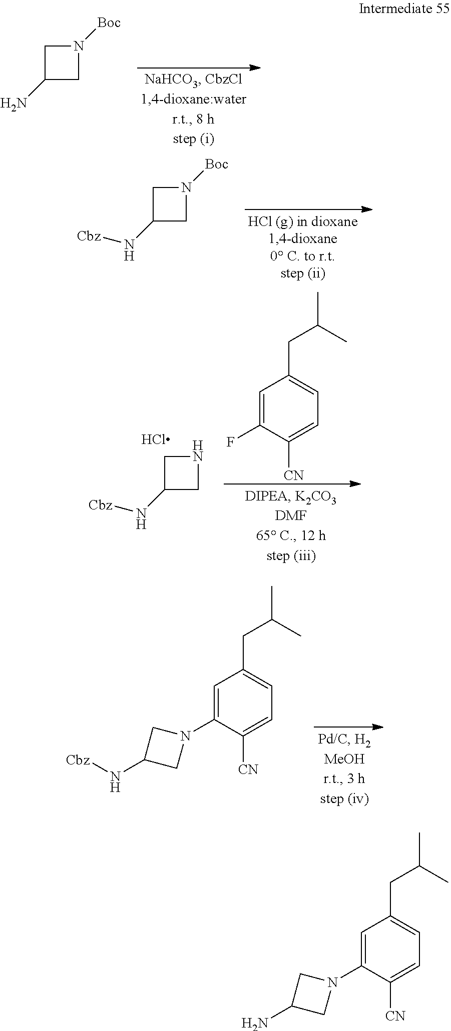

### Step (i): tert-Butyl 3-(benzyloxycarbonylamino)azetidine-1-carboxylate

To a stirred solution of tert-butyl 3-aminoazetidine-1-carboxylate (300 mg, 1.74 mmol) in a 1,4-dioxane:water mixture (7:3, 10 mL) at 0° C. was added NaHCO₃ (366 mg, 4.35 mmol), followed by a 50 wt % benzyl chloroformate solution in toluene (0.87 mL, 2.61 mmol). Upon completion of the addition, the reaction was allowed to warm up to room temperature and kept stirring at room temperature for 8 h. After the completion of the reaction was confirmed by TLC, the reaction mixture was concentrated to dryness and extraction with EtOAc and water was performed. The combined organic layers were washed with sat. brine, dried over anhydrous sodium sulfate and concentrated in vacuo. The obtained crude residue was purified by column chromatography over silica gel (30-35% EtOAc in hexane) to afford tert-butyl 3-(benzyloxycarbonylamino)azetidine-1-carboxylate as a gummy liquid (185 mg, 35%).

### Step (ii): Benzyl N-(azetidin-3-yl)carbamate hydrochloride

To a stirred solution of tert-butyl 3-(benzyloxycarbonylamino)azetidine-1-carboxylate (185 mg, 0.603 mmol) in 1,4-dioxane (1 mL) was added HCl (g) in dioxane (5 mL) at 0° C., after which the solution was slowly warmed up to room temperature and kept stirring at r.t. for an additional 5 hours. After the completion of the reaction was confirmed by TLC, the reaction mixture was evaporated to dryness under reduced pressure. The obtained crude hydrochloride salt was used as such in the following reaction (135 mg crude).

### Step (iii): Benzyl N-[1-(2-cyano-5-isobutyl-phenyl)azetidin-3-yl]carbamate

To a stirred solution of benzyl N-(azetidin-3-yl)carbamate hydrochloride (135 mg, 0.556 mmol) at room temperature in DMF (5 mL) was added DIPEA (0.24 mL, 1.390 mmol) and K₂CO₃ (154 mg, 1.112 mmol). Subsequently, 2-fluoro-4-isobutylbenzonitrile (109 mg, 0.612 mmol) was added to this mixture at room temperature, after which the reaction was brought to 65° C. for 12 h. The solution was cooled down, cold water was added and an extraction with EtOAc was performed. The combined organic layers were washed with sat. brine, dried over anhydrous sodium sulfate and concentrated under reduced pressure. The organic residue was purified by column chromatography over silica gel (2-3% MeOH in CH₂Cl₂) to yield the envisaged nitrile as a gummy liquid (100 mg, 46% over 2 steps).

### Step (iv): 2-(3-Aminoazetidin-1-yl)-4-isobutyl-benzonitrile

A stirred solution of benzyl N-[1-(2-cyano-5-isobutyl-phenyl)azetidin-3-yl]carbamate (100 mg, 0.275 mmol) in MeOH (5 mL) was hydrogenated over 10% Pd/C (40 mg) under 5 Kg/cm² H₂ pressure using a Parr hydrogenator for 3 h at ambient temperature. After confirming the completion of the reaction by LC-MS, the reaction mixture was filtered through a Celite® bed and was concentrated in vacuo. The desired hydrogenated compound was obtained as a colorless gum (51 mg, 80%) and used as such in the following reaction step.

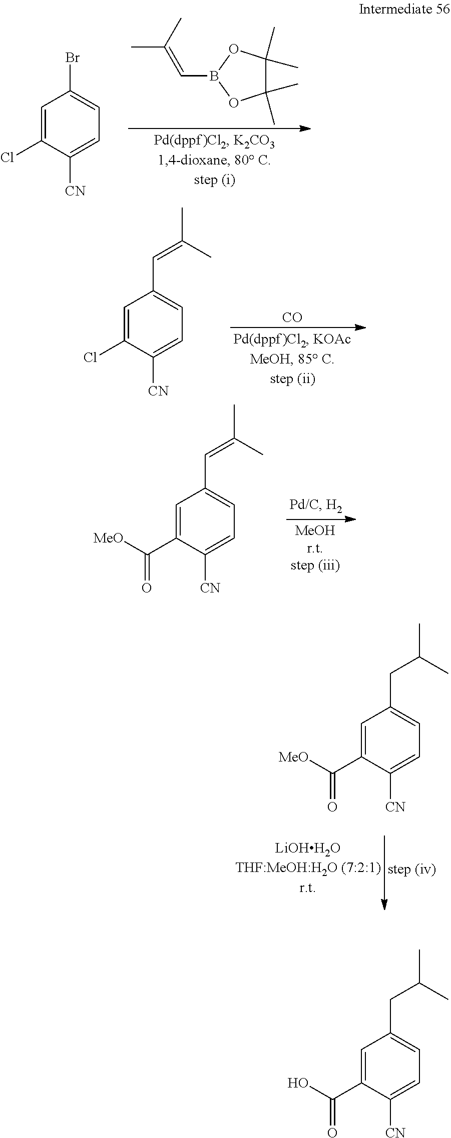

### Step (i): 2-Chloro-4-(2-methylprop-1-enyl)benzonitrile

Synthesized according to the described protocol mentioned in step (i) of intermediate 51 and intermediate 52.

### Step (ii): Methyl 2-cyano-5-(2-methylprop-1-enyl)benzoate

To a stirred solution of 2-chloro-4-(2-methylprop-1-enyl)benzonitrile (3.50 g, 18.3 mmol) in MeOH (25 mL) was added potassium acetate (4.48 g, 45.7 mmol), followed by Pd(dppf)Cl₂ (668 mg, 0.913 mmol). The resultant mixture was bubbled with argon for 20 min, after which the reaction was brought to 85° C. for 12 h under 5 Kg/cm² CO pressure using a Parr apparatus. After confirming full conversion of the reaction via TLC and LC-MS, the reaction mixture was concentrated under reduced pressure to remove the volatiles. The obtained crude residue was purified by column chromatography over silica gel (1-2% MeOH in CH₂Cl₂) to afford the envisaged methyl ester as a gummy liquid (983 mg, 25%).

### Step (iii): Methyl 2-cyano-5-isobutyl-benzoate

A stirred solution of methyl 2-cyano-5-(2-methylprop-1-enyl)benzoate (200 mg, 0.929 mmol) in MeOH (5 mL) was hydrogenated over 10% Pd/C (40 mg) under 5 Kg/cm² H₂ pressure using a Parr hydrogenator for 3 h at ambient temperature. After confirming the completion of the reaction by LC-MS, the reaction mixture was filtered through a Celite® bed and was concentrated in vacuo. The obtained colorless gum was used as such in the next step (168 mg, 83%).

### Step (iv): 2-Cyano-5-isobutyl-benzoic acid

To an ice-cold solution of methyl 2-cyano-5-isobutyl-benzoate (165 mg, 0.759 mmol) in THF:MeOH:H₂O (7:2:1, 10 mL) was added lithium hydroxide monohydrate (48 mg, 1.14 mmol) after which the solution was kept stirring at room temperature for 8 h. After the completion of the reaction was confirmed by TLC and LC-MS, the reaction mixture was concentrated under reduced pressure to remove the volatiles. Extraction with water and ethyl acetate was performed, after which the aqueous phase was neutralized with 1M HCl. The carboxylic acid was extracted into ethyl acetate, after which the organic layer was dried over anhydrous Na₂SO₄, filtered and concentrated in vacuo to obtain the crude carboxylic acid of interest as a colorless gum (122 mg, 79%).

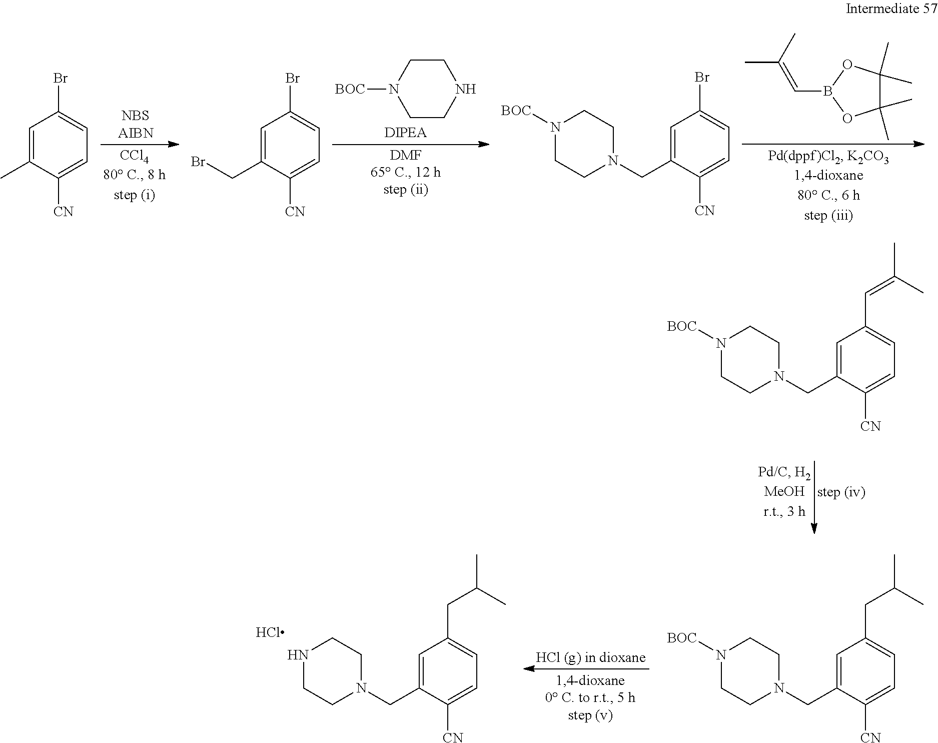

### Step (i): 4-Bromo-2-(bromomethyl)benzonitrile

To a stirred solution of 4-bromo-2-methyl-benzonitrile (8.00 g, 40.8 mmol) in CCl₄ (80 mL) was added NBS (7.99 g, 44.9 mmol), followed by AIBN (335 mg, 2.04 mmol). Upon completion of the addition, the resulting reaction mixture was heated to 80° C. for 8 h. After completion of the reaction was confirmed by TLC, the reaction mixture was cooled down. Extraction with cold water and ethyl acetate was performed, after which the combined organic layers were subsequently washed with water and sat. brine. Drying over anhydrous sodium sulfate and solvent evaporation under reduced pressure, yielded a crude residue, which was further purified by column chromatography over silica gel (20-30% EtOAc in hexane) to afford the desired halide as an off-white solid (2.02 g, 18%).

### Step (ii): tert-Butyl 4-[(5-bromo-2-cyano-phenyl)methyl]piperazine-1-carboxylate

To a stirred solution of Boc-protected piperazine (500 mg, 2.68 mmol) in DMF (8 mL) was added 4-bromo-2-(bromomethyl)benzonitrile (886 mg, 3.22 mmol), followed by DIPEA (1.17 mL, 6.71 mmol), after which the reaction was continued at 65° C. for 12 h. Upon completion of the reaction (confirmed by TLC), cold water was added to the reaction mixture and extraction with EtOAc was performed. The combined organic layers were washed with sat. brine, dried over anhydrous sodium sulfate and concentrated in vacuo. The obtained crude residue was purified by column chromatography (SiO₂, 2-3% MeOH in CH₂Cl₂) to yield tert-butyl 4-[(5-bromo-2-cyano-phenyl)methyl]piperazine-1-carboxylate as a gummy liquid (880 mg, 86%).

### Step (iii): tert-Butyl 4-[[2-cyano-5-(2-methylprop-1-enyl)phenyl]methyl]piperazine-1-carboxylate

To a stirred solution of tert-butyl 4-[(5-bromo-2-cyano-phenyl)methyl]piperazine-1-carboxylate (850 mg, 2.24 mmol) in 1,4-dioxane (10 mL) was added 4,4,5,5-tetramethyl-2-(2-methylprop-1-en-1-yl)-1,3,2-dioxaborolane (448 mg, 2.46 mmol), followed by K₂CO₃ (772 mg, 5.59 mmol) and the resultant mixture was bubbled with argon for 20 min. Then Pd(dppf)Cl₂ (164 mg, 0.224 mmol) was added after which the reaction was heated to 80° C. for 6 h. After the completion of reaction was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (15-20% EtOAc in hexane) afforded tert-butyl 4-[[2-cyano-5-(2-methylprop-1-enyl)phenyl]methyl]piperazine-1-carboxylate as a colorless gum (740 mg, 93%).

### Step (iv): tert-Butyl 4-[(2-cyano-5-isobutyl-phenyl)methyl]piperazine-1-carboxylate

A stirred solution of tert-butyl 4-[[2-cyano-5-(2-methylprop-1-enyl)phenyl]methyl]piperazine-1-carboxylate (730 mg, 2.054 mmol) in MeOH (20 mL) was hydrogenated over 10% Pd/C (80 mg) under 5 Kg/cm² H₂ pressure using a Parr hydrogenator for 3 h at ambient temperature. After confirming the completion of the reaction by LC-MS, the reaction mixture was filtered through a Celite® bed and was concentrated in vacuo. The obtained hydrogenated compound was used as such in the next step (colorless gum, 650 mg, 89%).

### Step (v): 4-Isobutyl-2-(piperazin-1-ylmethyl)benzonitrile hydrochloride

To a stirred solution of tert-butyl 4-[(2-cyano-5-isobutyl-phenyl)methyl]piperazine-1-carboxylate (550 mg, 1.54 mmol) in 1,4-dioxane (5 mL) was added HCl (g) in dioxane (10 mL) at 0° C., after which the solution was slowly warmed up to room temperature and kept stirring at r.t. for 5 h. After the completion of the reaction was confirmed by TLC, the reaction mixture was evaporated to dryness under reduced pressure. The obtained crude hydrochloride salt was used as such in the following reaction (450 mg crude).

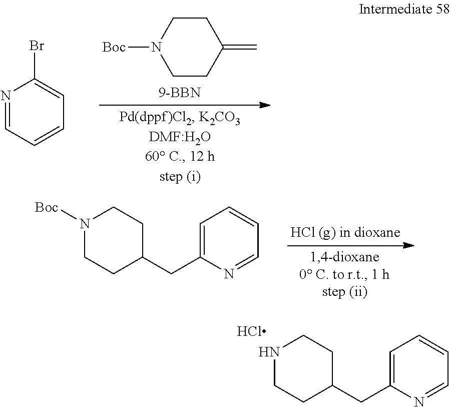 [SMILES: Brc1ccccn1 ; C=C1CCN(C(=O)OC(C)(C)C)CC1 ; CC(C)(C)OC(=O)N1CCC(Cc2ccccn2)CC1 ; c1ccc(CC2CCNCC2)nc1]

### Step (i): tert-Butyl 4-(2-pyridylmethyl)piperidine-1-carboxylate

A solution of tert-butyl 4-methylenepiperidine-1-carboxylate (1.50 g, 7.60 mmol) in 0.5M 9-BBN (15.2 mL, 7.60 mmol) was stirred for 1 h at 80° C., after which 2-bromopyridine (1.00 g, 6.33 mmol), Pd(dppf)Cl₂·CH₂Cl₂ (258 mg, 0.316 mmol) and K₂CO₃ (1.14 g, 8.23 mmol) in DMF:H₂O (9:1, 10 mL) were added. The resulting mixture was heated to 60° C. for 12 h. After the completion of the reaction was confirmed by TLC and LC-MS, the reaction mixture was poured into water and extracted with ethyl acetate. The combined organic layers were washed with sat. brine, dried over anhydrous sodium sulfate and concentrated in vacuo. Subsequent column chromatography over silica gel (10-20% EtOAc in hexane) afforded tert-butyl 4-(2-pyridylmethyl)piperidine-1-carboxylate as a colorless oil (997 mg, 57%).

### Step (ii): 2-(4-Piperidylmethyl)pyridine hydrochloride

A mixture of tert-butyl 4-(2-pyridylmethyl)piperidine-i-carboxylate (990 mg, 3.58 mmol) in 4M HCl (g) in dioxane (10 mL) at 0° C. was slowly warmed up to room temperature and kept stirring at r.t. under inert atmosphere for 1 h. After the completion of the reaction was confirmed by LC-MS, the reaction mixture was evaporated to dryness to give the envisaged hydrochloride salt as an off-white solid (747 mg, 98%).

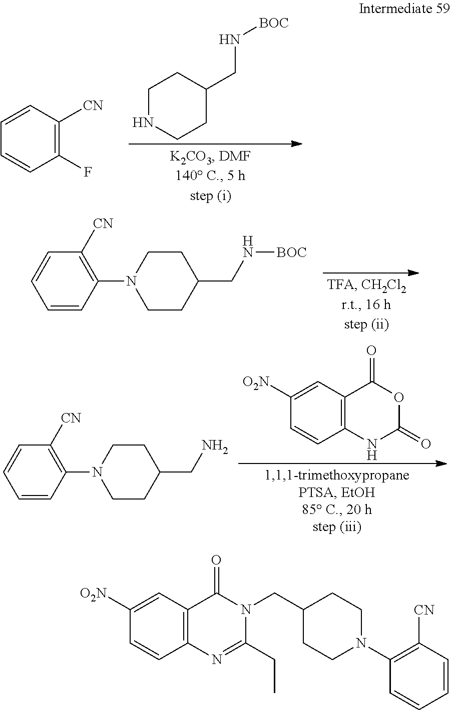 [SMILES: N#Cc1ccccc1F ; CC(C)(C)OC(=O)NCC1CCNCC1 ; CC(C)(C)OC(=O)NCC1CCN(c2ccccc2C#N)CC1 ; N#Cc1ccccc1N1CCC(CN)CC1 ; O=c1[nH]c2ccc([N+](=O)[O-])cc2c(=O)o1 ; CCc1nc2ccc([N+](=O)[O-])cc2c(=O)n1CC1CCN(c2ccccc2C#N)CC1]

### Step (i): tert-butyl N-[[1-(2-cyanophenyl)-4-piperidyl]methyl]carbamate

To a stirred solution of 2-fluorobenzonitrile (10.0 g, 82.6 mmol) in dry DMF (200 mL) was added tert-butyl (piperidin-4-ylmethyl)carbamate (21.2 g, 99.1 mmol), followed by K₂CO₃ (22.8 g, 165.1 mmol) after which the solution was brought to 140° C. for 5 h. After the completion of the reaction was confirmed by TLC, the reaction mixture was poured into ice water and extraction with EtOAc was performed. The combined organic layers were washed with sat. brine, dried over sodium sulfate and concentrated under reduced pressure. Subsequent column chromatography of the obtained crude residue on silica gel (15-20% EtOAc in hexane) afforded tert-butyl N-[[1-(2-cyanophenyl)-4-piperidyl]methyl]carbamate as a colorless solid (17.7 g, 68%).

### Step (ii): 2-[4-(aminomethyl)-1-piperidyl]benzonitrile

To an ice-cold solution of tert-butyl N-[[1-(2-cyanophenyl)-4-piperidyl]methyl]carbamate (13.6 g, 43.1 mmol) in anhydrous methylene chloride (300 mL) was carefully added trifluoroacetic acid (4.95 mL, 64.7 mmol). Upon completion of the addition, the reaction was allowed to slowly warm up to room temperature and kept stirring at this temperature for 16 h. When complete conversion of the reaction was confirmed by TLC, a sat. NaHCO₃ solution was added to neutralize the reaction mixture and extraction with CH₂Cl₂ was performed. The combined organic layers were washed with sat. brine, dried over anhydrous Na₂SO₄ and concentrated in vacuo to deliver 2-[4-(aminomethyl)-1-piperidyl]benzonitrile as a pale-yellow liquid (7.98 g, 86%).

### Step (iii): 2-[4-[(2-ethyl-6-nitro-4-oxo-quinazolin-3-yl)methyl]-1-piperidyl]benzonitrile

To a stirred solution of 2-[4-(aminomethyl)-1-piperidyl]benzonitrile (4.00 g, 18.6 mmol) in anhydrous EtOH (10 mL) was added 6-nitro-1H-3,1-benzoxazine-2,4-dione (3.87 g, 18.6 mmol) and PTSA (320 mg, 1.86 mmol) after which the reaction was stirred at 85° C. for 2 h. At this point, 1,1,1-trimethoxypropane (22.2 mL, 149.6 mmol) was added and stirring was continued at 85° C. for an additional 18 hours. Upon completion of the reaction (as determined by TLC), the reaction mixture was cooled down to 0° C. and the resulting precipitate was filtered off to yield 2-[4-[(2-ethyl-6-nitro-4-oxo-quinazolin-3-yl)methyl]-1-piperidyl]benzonitrile as a yellow solid (4.19 g, 54%).

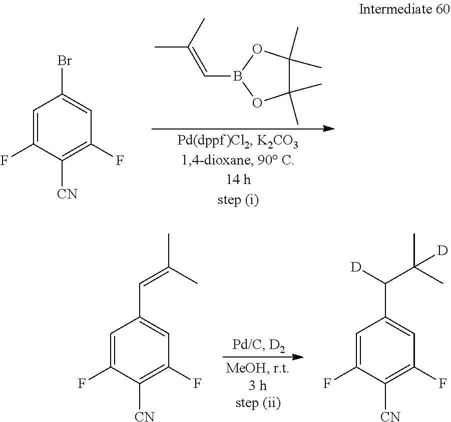 [SMILES: N#Cc1c(F)cc(Br)cc1F ; CC(C)=CB1OC(C)(C)C(C)(C)O1 ; CC(C)=Cc1cc(F)c(C#N)c(F)c1 ; [2H]C(c1cc(F)c(C#N)c(F)c1)C([2H])(C)C]

Intermediate 60, a deuterated analog of 2,6-difluoro-4-(2-methylprop-1-en-1-yl)benzonitrile, intermediate 10) which was used as an intermediate for a number of compounds of the invention, was prepared as follows:

### Step (i): 2,6-Difluoro-4-(2-methylprop-1-en-1-yl)benzonitrile

To a stirred solution of 4-bromo-2,6-difluorobenzonitrile (5.0 g, 22.93 mmol) in 1,4-dioxane (75 mL) was added 4,4,5,5-tetramethyl-2-(2-methylprop-1-en-1-yl)-1,3,2-dioxaborolane (5.01 g, 27.52 mmol), followed by K₂CO₃ (7.92 g, 57.34 mmol) and the resultant mixture was bubbled with argon for 20 min. Then Pd(dppf)Cl₂ (0.839 g, 1.147 mmol) was added after which the reaction was heated to 90° C. for 14 h. After the completion of reaction was confirmed by TLC, the reaction mixture was evaporated under reduced pressure to remove the volatiles. The residue was dissolved with ethyl acetate and washed with water, sat. brine, dried over anhydrous sodium sulfate and evaporated under reduced pressure to afford a crude residue. The crude compound thus obtained was purified by column chromatography over silica gel, eluting with 15-20% EtOAc in hexane, to afford 2,6-difluoro-4-(2-methylprop-1-en-1-yl)benzonitrile as a colorless gum (3.6 g, 81%).

### Step (ii): 4-(1,2-Dideuterio-2-methyl-propyl)-2,6-difluoro-benzonitrile

A stirred solution of 2,6-difluoro-4-(2-methylprop-1-en-1-yl)benzonitrile (1 g, 5.18 mmol) in MeOH (10 mL) was hydrogenated over 10% Pd/C (0.10 g) under 2 Kg/cm² D₂ pressure using a Parr hydrogenator for 3 h at ambient temperature. After confirming the completion of reaction by LC-MS, the reaction mixture was filtered through a Celite® bed and was evaporated in vacuo. The crude residue was purified by silica chromatography using 10-13% EtOAc in hexane to afford 4-(1,2-dideuterio-2-methyl-propyl)-2,6-difluoro-benzonitrile as a pale yellow liquid (0.92 g, 90%).

### General Synthetic Methods for Preparation of the Compounds of the Invention

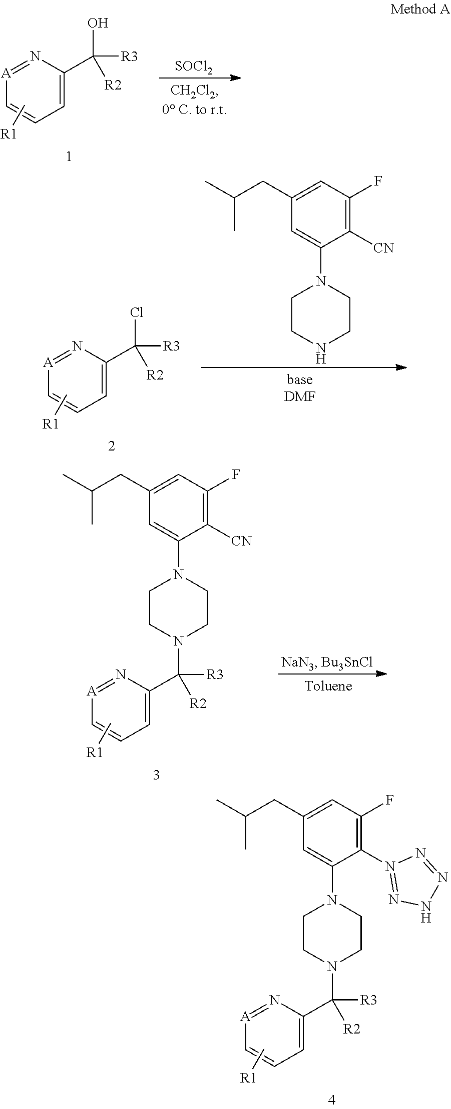

To a stirred solution of compound 1 (1 equiv.) in CH₂Cl₂ was added SOCl₂ (2.5 equiv.) at 0° C. Upon completion of the addition, the reaction was allowed to reach room temperature and was kept stirring at this temperature for 1 to 3 hours. After completion of the reaction was confirmed by TLC, the reaction mixture was evaporated to dryness, neutralized with a cold sodium bicarbonate solution and extracted with CH₂Cl₂. The combined organic layers were washed with sat. brine, dried over anhydrous sodium sulfate and evaporated under reduced pressure to get a crude residue, which was taken to the next step without additional purification.

To a stirred solution of intermediate 2 (1 equiv.) in DMF at 0° C. was added DIPEA or K₂CO₃ (2 to 2.5 equiv.) after which the solution was stirred at r.t. for 10 minutes. A heteroaryl chloride of interest 2 (1.2 equiv.) was added and the reaction was continued at r.t. overnight. After completion of the reaction was confirmed by TLC, the reaction mixture was diluted with cold water and extracted with EtOAc. The combined organic layers were washed with sat. brine, dried over anhydrous sodium sulfate and evaporated under reduced pressure. The crude thus obtained was purified by column chromatography over silica gel, eluting with 2-3% of MeOH in CH₂Cl₂.

A mixture of the nitrile 3 (1 equiv.), NaN₃ (5 to 8 equiv.) and Bu₃SnCl (5 to 8 equiv.) in toluene was stirred at 140° C. in a sealed tube until completion of the reaction was confirmed by TLC. The reaction mixture was evaporated under reduced pressure and the thus obtained residue was re-dissolved in CH₂Cl₂. The organic phase was washed with a 10% NaOH solution. The aqueous layer was then neutralized with a citric acid solution and extracted with CH₂Cl₂. The combined organic layers were washed with sat. brine, dried over anhydrous sodium sulfate and evaporated in vacuo. The crude compound was purified by silica chromatography, eluting the envisaged compound with 4-5% of MeOH in CH₂Cl₂.

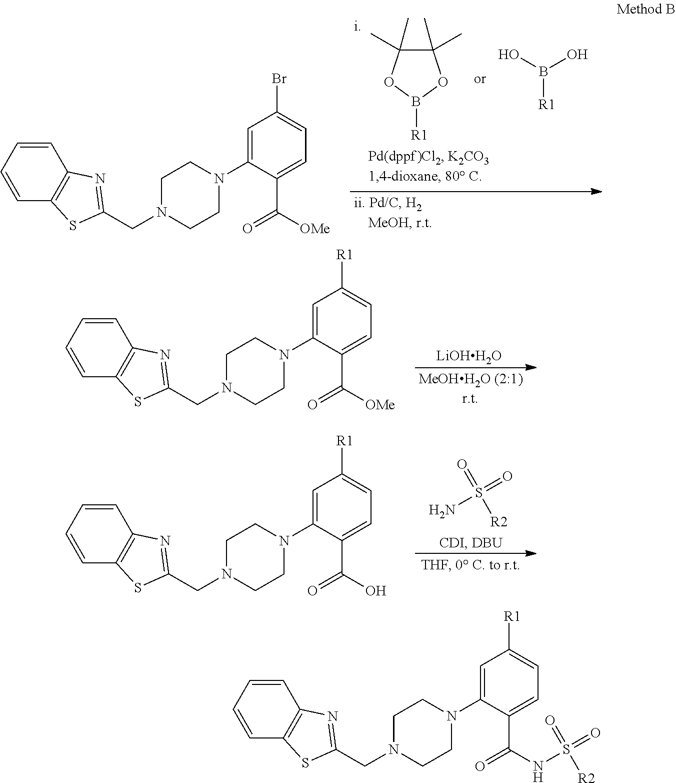

To a stirred solution of intermediate 3 (1 equiv.) in 1,4-dioxane was added a substituted boronic acid or boronic pinacol ester of interest (1.2 equiv), followed by K₂CO₃ (2.5 equiv.) and the resultant mixture was bubbled with argon for 20 min. Then Pd(dppf)Cl₂ (0.1 equiv.) was added after which the reaction was heated to 80° C. until completion of the reaction was observed by TLC. The reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel yielded the desired compound.

When needed, a solution of a substituted 2-(4-(benzo[d]thiazol-2-ylmethyl)piperazin-1-yl)benzoate (1 equiv.) in MeOH was hydrogenated over 10% Pd/C under 5 Kg/cm² H₂ pressure using a Parr hydrogenator at ambient temperature. The reaction mixture was filtered over a Celite Bed®, optionally followed by a trituration in an appropriate solvent (e.g. diethyl ether).

Following the hydrogenation, a solution of substituted methyl 2-(4-(benzo[d]thiazol-2-ylmethyl)piperazin-1-yl)benzoate (1 equiv.) and lithium hydroxide monohydrate (2 equiv.) in MeOH:H₂O (2:1 ratio) was stirred at room temperature. After completion of the hydrolysis reaction, as determined by TLC and LC-MS, the mixture was concentrated under reduced pressure to get rid of solvents. The organic residue was diluted with water and back washed with ethyl acetate. The aqueous phase was subsequently neutralized with 1M citric acid and extraction with ethyl acetate was performed. The combined organic layers were dried over anhydrous Na₂SO₄, filtered and concentrated under reduced pressure to afford the carboxylic acid which was used as such in the next step without any purification.

To a solution of the carboxylic acid (1 equiv.) in THE was added CDI (2 equiv.) at 0° C., after which the reaction was left stirring at room temperature for 15 minutes. Next, DBU (2 equiv.) and a substituted sulfonamide of interest (1.2 equiv.) were added, after which the reaction mixture was kept stirring at room temperature overnight. After confirmation of the completion of the reaction by TLC, the solution was evaporated to dryness, diluted with water and extracted with ethyl acetate. The combined organic layers were washed with sat. brine, dried over anhydrous sodium sulfate and evaporated in vacuo. Finally, column chromatography over silica gel using a mixture of MeOH in CH₂Cl₂ allowed isolation of the envisaged compound.

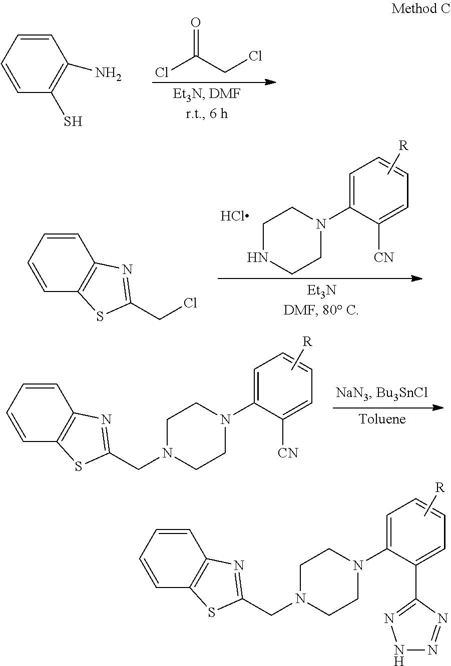

A solution of 2-aminobenzenethiol (1.0 g, 8.0 mmol) and Et₃N (3.32 mL, 24.0 mmol) in DMF (25 mL) was stirred at 0° C., followed by careful addition of 2-chloroacetyl chloride (1.35 g, 12.0 mmol). Upon completion of the addition, the resultant mixture was allowed to warm up to room temperature and kept stirring at r.t. for 6 h. After the completion of the reaction was confirmed by TLC, the reaction mixture was diluted with water and extracted with ethyl acetate. The combined organic layers were washed with water, sat. brine, dried over anhydrous sodium sulfate and evaporated under reduced pressure. The crude 2-(chloromethyl)benzo[d]thiazole was used in the following step without additional purification (1.22 g, crude).

To a stirred solution of a substituted nitrile of interest (1 equiv.) in DMF at 0° C. was added triethylamine (2.5-3 equiv.) after which the reaction was stirred at room temperature for 10 minutes. Subsequently, 2-(chloromethyl)benzo[d]thiazole (1.5 equiv.) was added and the reaction was continued at 80° C. After completion of the reaction was confirmed by TLC, the reaction mixture was diluted with cold water and extracted with EtOAc. The combined organic layers were washed with water, sat. brine, dried over anhydrous sodium sulfate and evaporated under reduced pressure. The crude obtained was purified by column chromatography over silica gel.

A mixture of the nitrile containing intermediate (1 equiv.), NaN₃ (5 to 8 equiv.) and Bu₃SnCl (5 to 8 equiv.) in toluene was stirred at 150° C. in a sealed tube. After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layer was washed with sat. brine, dried over anhydrous sodium sulfate and evaporated under reduced pressure. The crude compound was purified by column chromatography over silica gel.

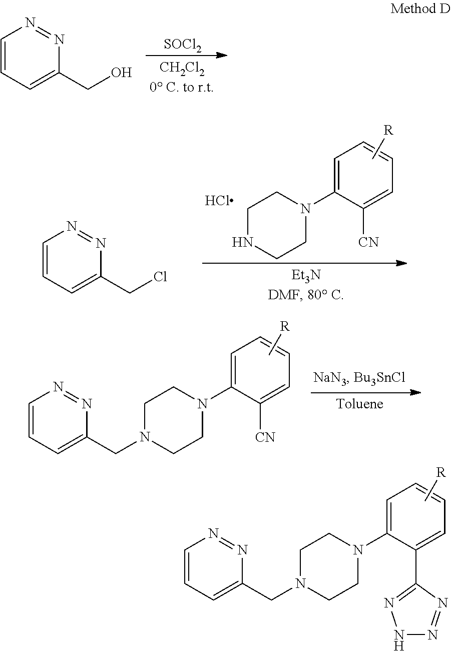

To a stirred solution of 3-(hydroxymethyl)pyridazine (30 g, 272.72 mmol) in CH₂Cl₂ (300 mL) was added SOCl₂ (48.46 mL, 681.81 mmol) at 0° C. and the reaction was allowed to stir for 1 h. After completion of the reaction was confirmed by TLC, the reaction mixture was diluted with a cold sat. NaHCO₃ solution and extracted with CH₂Cl₂. The combined organic layer was washed with sat. brine, dried over anhydrous sodium sulfate and evaporated under reduced pressure. The crude 3-(chloromethyl)pyridazine (31 g, 88%) thus obtained was used in the next step without further purification.

To a stirred solution of a substituted nitrile of interest (1 equiv.) in DMF at 0° C. was added triethylamine (3 equiv.) after which the reaction was stirred at room temperature for 10 minutes. Subsequently, 3-(chloromethyl)pyridazine (1.5 equiv.) was added and the reaction was continued at 80° C. After completion of the reaction was confirmed by TLC, the reaction mixture was diluted with cold water and extracted with EtOAc. The combined organic layers were washed with water, sat. brine, dried over anhydrous sodium sulfate and evaporated under reduced pressure. The crude obtained was purified by column chromatography over silica gel.

A mixture of the nitrile containing intermediate (1 equiv.), NaN₃ (5 to 8 equiv.) and Bu₃SnCl (5 to 8 equiv.) in toluene was stirred at 150° C. in a sealed tube. After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layer was washed with sat. brine, dried over anhydrous sodium sulfate and evaporated under reduced pressure. The crude compound was purified by column chromatography over silica gel.

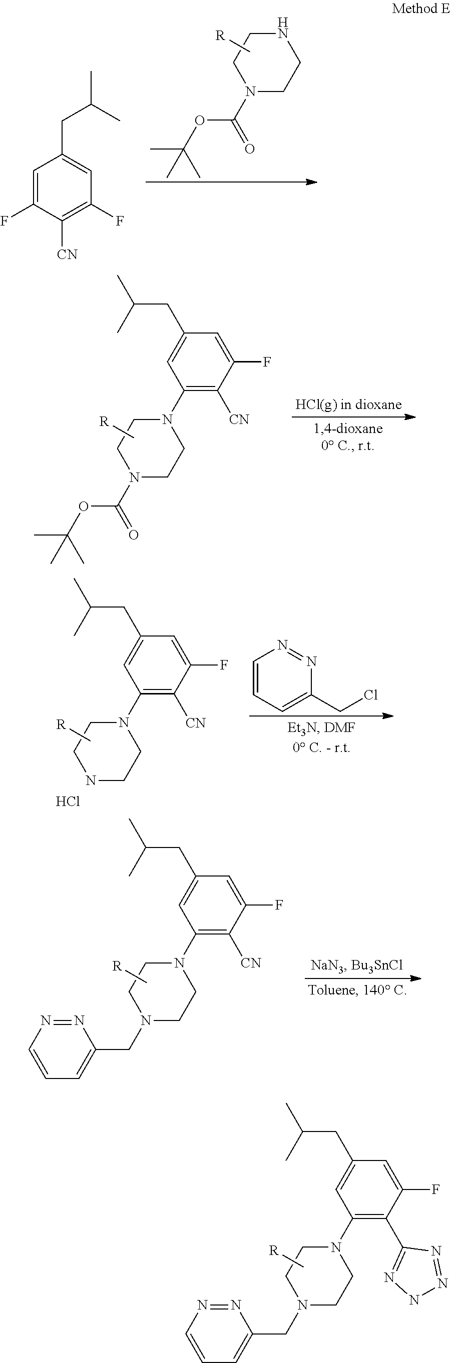

To a stirred solution at room temperature of a substituted piperazine of interest (1.1-1.2 equiv.) in DMF or DMSO was added K₂CO₃ (3 equiv.) or DIPEA (2.5 to 3 equiv.). Subsequently, 2,6-difluoro-4-isobutylbenzonitrile (1 equiv.) was added to this mixture at room temperature, after which the reaction was brought to 80-100° C. until completion of the reaction was confirmed by TLC. The solution was cooled down, diluted with cold water and an extraction with CH₂Cl₂ was performed. The combined organic layers were washed with sat. brine, dried over anhydrous sodium sulfate and evaporated under reduced pressure. The organic residue was purified by column chromatography over silica gel.

In a next step, the isolated SNAr product (1 equiv.) was dissolved in 1,4-dioxane to which HCl (g) in dioxane was added at 0° C. Upon completion of the addition, the reaction was allowed to slowly warm up to room temperature and kept stirring until complete conversion was obtained (via TLC). The reaction mixture was concentrated in vacuo, followed by washing of the crude residue with hexane. The crude compound was taken to the next step without additional purification.

To a stirred solution of nitrile (1 equiv.) in DMF at 0° C. was added triethylamine (3 to 3.5 equiv.) or K₂CO₃ (2 equiv.) dropwise, after which the reaction was stirred at room temperature for 10 minutes. Subsequently, 3-(chloromethyl)pyridazine (1.2 equiv.) was added and the reaction was continued at room temperature. After completion of the reaction was confirmed by TLC, the reaction mixture was diluted with cold water and extracted with CH₂Cl₂. The combined organic layers were washed with water, sat. brine, dried over anhydrous sodium sulfate and evaporated under reduced pressure. The crude obtained was purified by column chromatography over silica gel.

A mixture of the nitrile containing intermediate (1 equiv.), NaN₃ (5 to 8 equiv.) and Bu₃SnCl (5 to 8 equiv.) in toluene was stirred at 140-150° C. in a sealed tube. After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layer was washed with sat. brine, dried over anhydrous sodium sulfate and evaporated under reduced pressure. The crude compound was purified by column chromatography over silica gel.

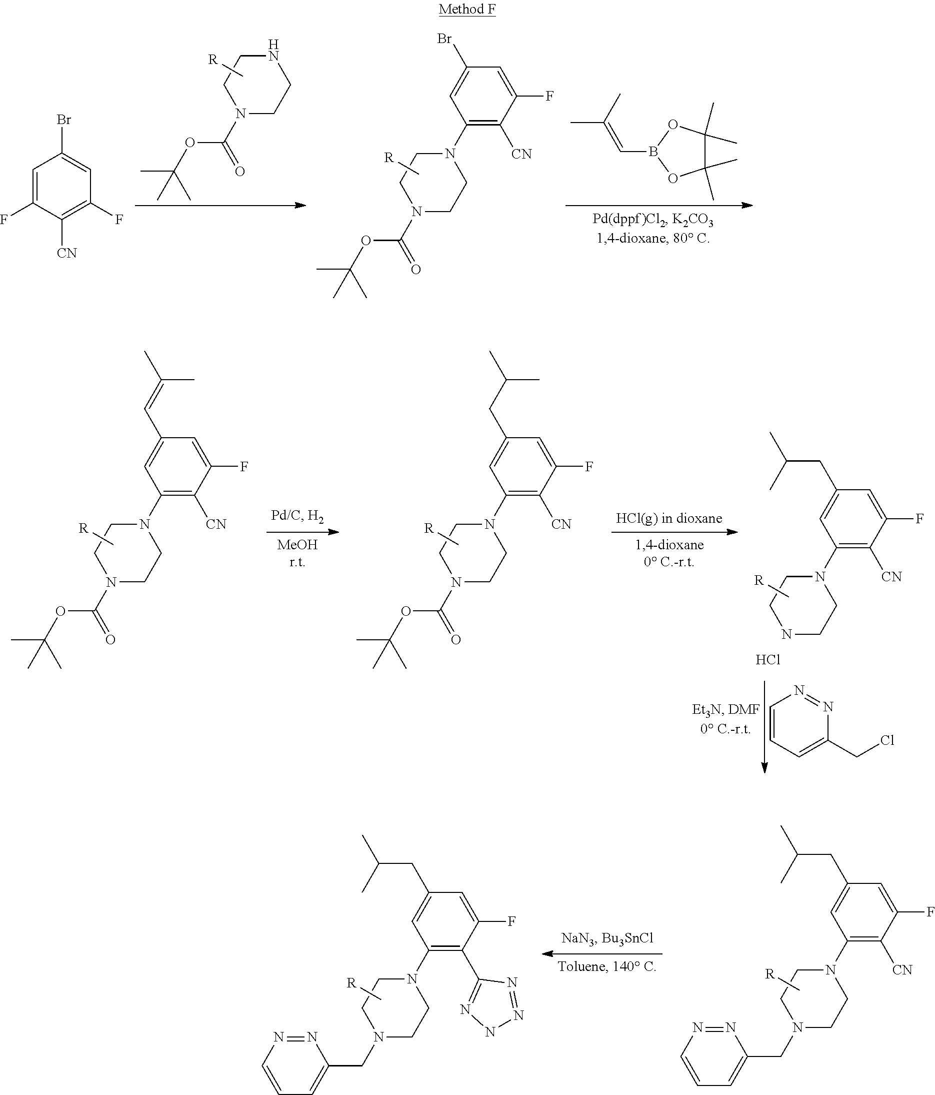

To a stirred solution at room temperature of a substituted piperazine of interest (1.2 equiv.) in DMF or DMSO was added K₂CO₃ or DIPEA (2.5-3 equiv.). Subsequently, 2,6-difluoro-4-isobutylbenzonitrile (1 equiv.) was added to this mixture at room temperature, after which the reaction was brought to 80-100° C. until completion of the reaction was confirmed by TLC. The solution was cooled down, diluted with cold water and an extraction with CH₂Cl₂ was performed. The combined organic layers were washed with sat. brine, dried over anhydrous sodium sulfate and evaporated under reduced pressure. The organic residue was purified by column chromatography over silica gel.

To a stirred solution of isolated SNAr product (1 equiv.) in 1,4-dioxane was added 4,4,5,5-tetramethyl-2-(2-methylprop-1-en-1-yl)-1,3,2-dioxaborolane (1.2 equiv.), followed by K₂CO₃ (2.5 equiv.) and the resultant mixture was bubbled with argon for 20 min. Then Pd(dppf)Cl₂ (0.1 equiv.) was added after which the reaction was heated to 80° C. until completion of reaction was observed by TLC. The reaction mixture was concentrated under reduced pressure to remove the volatiles and the residue was re-dissolved with ethyl acetate and washed with water and sat. brine. Subsequent drying over anhydrous sodium sulfate and evaporation under reduced pressure, followed by column chromatography over silica gel, afforded the compound of interest.

Following the Suzuki coupling, a hydrogenation using a Parr hydrogenator at ambient temperature was performed. Therefore, the Suzuki product (1 equiv.) was dissolved in MeOH in the presence of 10% Pd/C while putting the reaction under 5 Kg/cm² H₂ pressure. After confirming the completion of the reaction by LC-MS, the reaction mixture was filtered through a Celite® bed and was concentrated in vacuo, yielding the envisaged hydrogenated compound. The crude compound was used as such in the following deprotection.

In a next step, the isolated compound (1 equiv.) was dissolved in 1,4-dioxane to which HCl (g) in dioxane was added at 0° C. Upon completion of the addition, the reaction was allowed to slowly warm up to room temperature and kept stirring until complete conversion was obtained (via TLC). The reaction mixture was concentrated in vacuo, followed by washing of the crude residue with hexane. The crude compound was taken to the next step without additional purification.

To a stirred solution of the deprotected compound (1 equiv.) in DMF at 0° C. was added triethylamine (3 to 4 equiv.) or DIPEA (2.5 to 3 equiv.) dropwise or Cs₂CO₃ (1.5 equiv.), after which the reaction was continued at room temperature for 10 minutes. Subsequently, 3-(chloromethyl)pyridazine (1.2 equiv.) was added and the reaction was continued at room temperature or heated to 60-80° C. After completion of the reaction was confirmed by TLC, the reaction mixture was diluted with cold water and extracted with CH₂Cl₂. The combined organic layers were washed with water, sat. brine, dried over anhydrous sodium sulfate and evaporated under reduced pressure. The crude obtained was purified by column chromatography over silica gel.

In a final reaction, a mixture of the nitrile containing intermediate (1 equiv.), NaN₃ (5 to 8 equiv.) and Bu₃SnCl (5 to 8 equiv.) in toluene was stirred at 140° C. in a sealed tube. After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layer was washed with sat. brine, dried over anhydrous sodium sulfate and evaporated under reduced pressure. The crude compound was purified by column chromatography over silica gel.

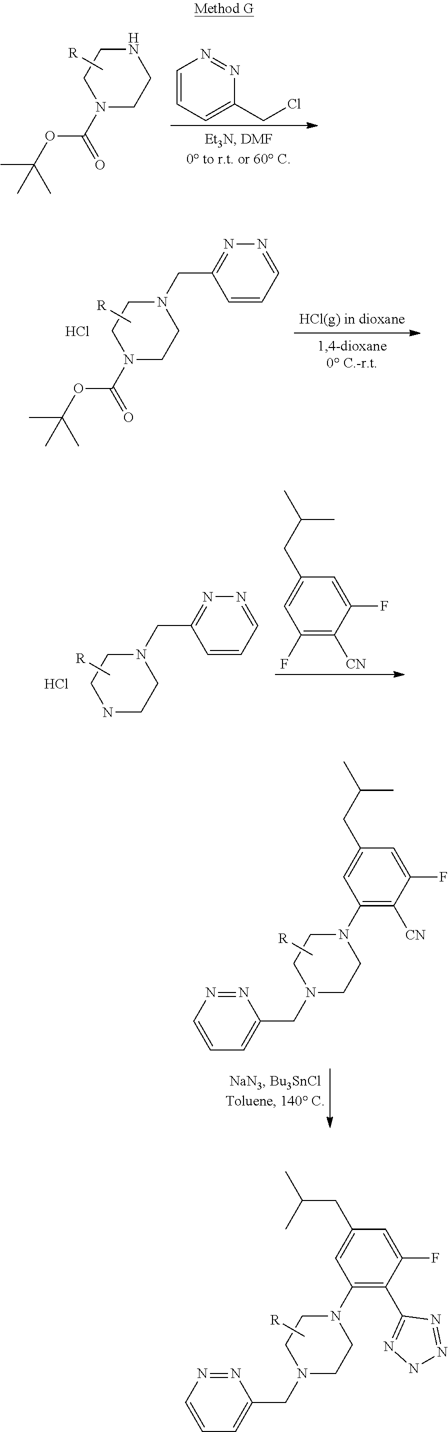

To a stirred solution of the substituted piperazine of interest (1 equiv.) in DMF at 0° C. was added triethylamine or DIPEA (3 to 4 equiv.) dropwise, after which the reaction was continued at room temperature for 10 minutes. Subsequently, 3-(chloromethyl)pyridazine (1.2 equiv.) was added after which the reaction was continued and brought to 60° C. when necessary to push conversion. After completion of the reaction was confirmed by TLC, the reaction mixture was diluted with cold water and extracted with CH₂Cl₂. The combined organic layers were washed with water, sat. brine, dried over anhydrous sodium sulfate and evaporated under reduced pressure. The crude obtained was purified by column chromatography over silica gel.

Following the alkylation reaction, the isolated compound (1 equiv.) was dissolved in 1,4-dioxane to which HCl (g) in dioxane was added at 0° C. Upon completion of the addition, the reaction was allowed to slowly warm up to room temperature and kept stirring until complete conversion was obtained (via TLC). The reaction mixture was concentrated in vacuo, followed by washing of the crude residue with hexane. The crude compound was taken to the next step without additional purification.

The isolated compound was taken into a SNAr reaction. Therefore, to a stirred solution at room temperature of the deprotected compound (1.0 equiv.) in DMF or DMSO was added DIPEA and/or K₂CO₃ (2.5 equiv. for each base, respectively). Subsequently, 2,6-difluoro-4-isobutylbenzonitrile (1.1 equiv.) was added to this mixture at room temperature, after which the reaction was brought to 60-80° C. until completion of the reaction was confirmed by TLC. The solution was cooled down, diluted with cold water and an extraction with CH₂Cl₂ was performed. The combined organic layers were washed with sat. brine, dried over anhydrous sodium sulfate and evaporated under reduced pressure. The organic residue was purified by column chromatography over silica gel.

In a final stage, a mixture of the nitrile containing intermediate (1 equiv.), NaN₃ (5 to 8 equiv.) and Bu₃SnCl (5 to 8 equiv.) in toluene was stirred at 140° C. in a sealed tube. After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layer was washed with sat. brine, dried over anhydrous sodium sulfate and evaporated under reduced pressure. The crude compound was purified by column chromatography over silica gel.

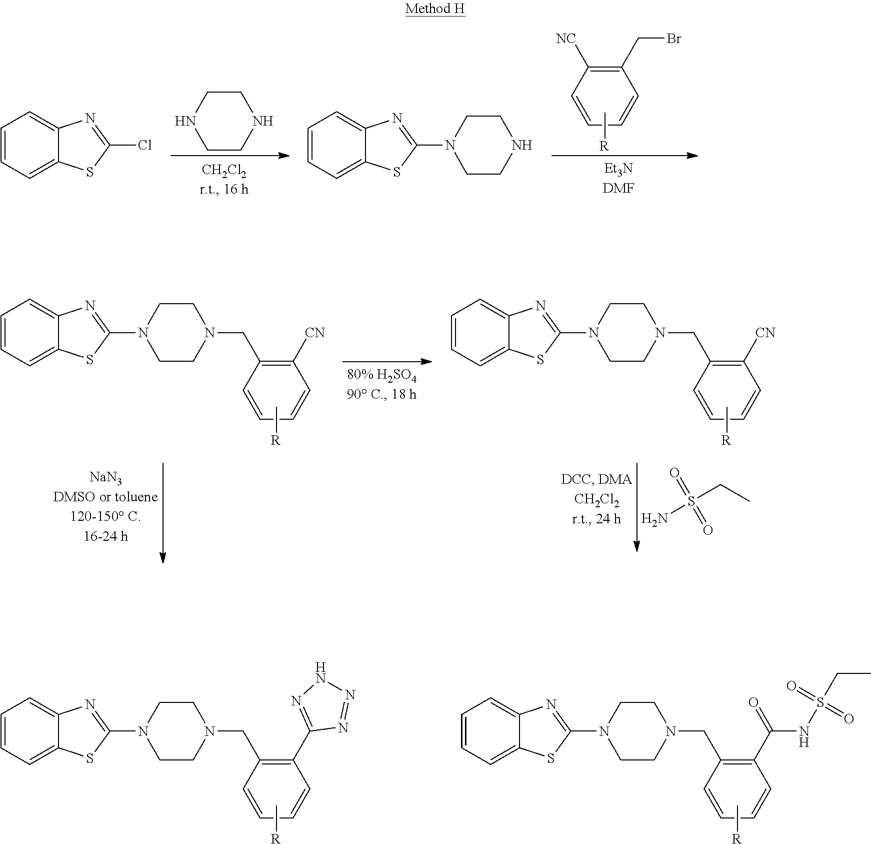

To a solution of 2-chloro-1,3-benzothiazole (2.0 g, 11.80 mmol) in dry methylene chloride (20 mL) was added piperazine (6.1 g, 71.00 mmol) and the solution was stirred at room temperature for 16 h. After this time, the reaction mixture was poured into ice water and extracted with methylene chloride. The organic layer was washed with sat. brine, dried over sodium sulfate, filtered and concentrated under reduced pressure to give the desired compound (2.5 g, 96%) as a colorless solid which was directly used in the next step without any further purification.

To a solution of 2-piperazin-1-yl-1,3-benzothiazole (100 mg, 0.45 mmol) in anhydrous DMF (10 mL) at 0° C. was added triethylamine (0.132 mL, 0.95 mmol) and a substituted 2-(bromomethyl)benzonitrile of interest (0.45 mmol), after which the reaction was kept stirring at room temperature or 100° C. Upon completion of the reaction as determined by TLC, the reaction mixture was poured into ice water and extracted with methylene chloride or ethyl acetate. The organic layer was washed with sat. brine, dried over sodium sulfate, filtered and concentrated in vacuo to give a residue which was purified on a column of silica gel.

A solution of the obtained nitrile (1.34 mmol) in 80% sulfuric acid (5 mL) was stirred at 90° C. for 18 h, after which the reaction mixture was poured into ice water and the pH was adjusted to ˜5 with 2M aqueous sodium hydroxide. The aqueous phase was extracted with methylene chloride, washed with sat. brine, dried over sodium sulfate, filtered and concentrated under reduced pressure to give a residue which was purified using column chromatography (silica gel, 5-10% methanol in methylene chloride).

To a solution of a substituted 2-[[4-(1,3-benzothiazol-2-yl)piperazin-1-yl]methyl]benzoic acid (0.33 mmol) in dry methylene chloride (5 mL) at r.t. was added DCC (700 mg, 3.3 mmol), followed by DMAP (41 mg, 0.33 mmol) and ethanesulfonamide (36 mg, 0.37 mmol) after which the solution was stirred at room temperature for 24 h. After this time, the reaction mixture was poured into ice water and extracted with methylene chloride. The organic layer was washed with 10% aqueous citric acid, sat. brine, dried over sodium sulfate, filtered and concentrated under reduced pressure. The obtained residue was further purified on a column of silica gel using EtOAc in hexane.

To a solution of a substituted 2-[[4-(1,3-benzothiazol-2-yl)piperazin-1-yl]methyl]benzonitrile (0.15 mmol) in DMSO or toluene (2-5 mL) was added NaN₃ (79 mg, 1.22 mmol) and Bu₃SnCl (0.33 mL, 1.22 mmol) after which the reaction was brought to 120-150° C. for 16-24 h. After this time, the reaction mixture was poured into ice water and extracted with methylene chloride. The organic layer was washed with a 10% NaOH solution and subsequently, the aqueous layer was neutralized with a citric acid solution. Extraction of the aq. phase with CH₂Cl₂ was performed. The combined organic layers were washed with sat. brine, dried over anhydrous sodium sulfate and evaporated under reduced pressure to afford the crude residue. The residual solid was further purified by column chromatography over silica gel, eluting with 5-10% of MeOH in CH₂Cl₂.

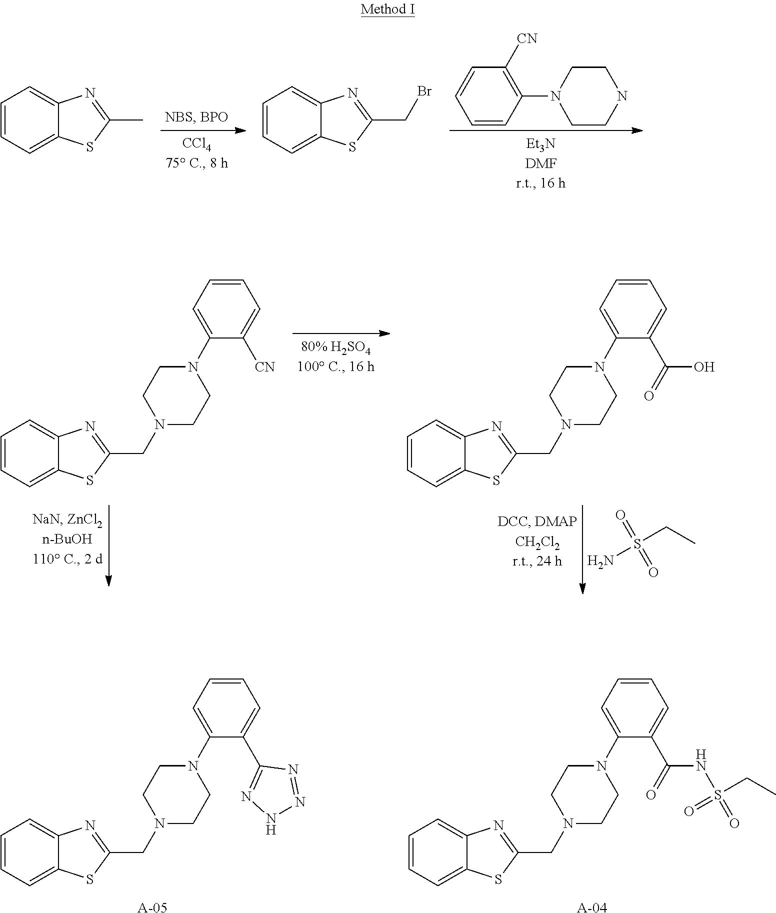

To a solution of 2-methyl-1,3-benzothiazole (100 mg, 1.30 mmol) in dry carbon tetrachloride (4 mL) was added NBS (238 mg, 1.30 mmol), benzoyl peroxide (62 mg, 0.20 mmol) and the reaction was stirred at 75° C. for 8 h. After this time, the reaction mixture was poured into ice water and extracted with methylene chloride. The organic layer was washed with sat. brine, dried over sodium sulfate, filtered and concentrated to dryness to give the heteroaryl bromide which was directly used in the next step without any further purification.

2-(Bromomethyl)benzo[d]thiazole (82 mg, 0.4 mmol) was slowly added to a solution of 2-piperazin-1-ylbenzonitrile (100 mg, 0.4 mmol) in dry DMF (2.5 mL) at 0° C., together with triethylamine (130 mg, 0.13 mmol). Upon completion of the addition, the reaction was allowed to slowly warm up to room temperature and kept stirring for 16 h. When the reaction had completed, the mixture was poured into ice water and extracted with methylene chloride. The organic layer was washed with sat. brine, dried over sodium sulfate, filtered and concentrated under reduced pressure to give a residual solid which was purified on a column of silica gel (20% EtOAc/80% hexane) (colorless solid, 15 mg, 11%).

A solution of 2-[4-(1,3-benzothiazol-2-ylmethyl)piperazin-1-yl]benzonitrile (400 mg, 1.20 mmol) in 80% H₂SO₄ (2.8 mL) was stirred at 100° C. for 16 h. Next, the reaction mixture was poured into ice water and the pH was adjusted to ˜5 with 2M aqueous NaOH solution. The aqueous phase was extracted with methylene chloride, washed with sat. brine, dried over sodium sulfate, filtered and concentrated in vacuo. Column chromatography (SiO₂, 80% EtOAc/20% hexane) yielded the carboxylic acid (250 mg, 59%) as a colorless solid.

To a solution of the obtained carboxylic acid (100 mg, 0.2 mmol) in dry methylene chloride (5 mL) was added DCC (580 mg, 2.8 mmol), followed by DMAP (345 mg, 2.8 mmol) and ethanesulfonamide (24 mg, 0.24 mmol), after which the suspension was stirred at room temperature for 24 h. When the reaction indicated full conversion by TLC, the reaction mixture was poured into ice water and extracted with methylene chloride. The organic layer was washed with 10% aqueous citric acid, sat. brine, dried over sodium sulfate, filtered and concentrated to dryness. The residual solid was purified on a column of silica gel (50% ethyl acetate/50% hexanes) to give 2-[4-(1,3-benzothiazol-2-ylmethyl)piperazin-1-yl]-N-ethylsulfonyl-benzamide A-04 (20 mg, 22%) as a colorless solid.

To a solution of 2-[4-(1,3-benzothiazol-2-ylmethyl)piperazin-1-yl]benzonitrile (100 mg, 0.20 mmol) in n-butanol (3 mL) was added NaN₃ (194 mg, 2.90 mmol) and a catalytic amount of ZnCl₂ (27 mg, 0.20 mmol) after which the suspension was stirred at 110° C. for 2 days. Upon completion, the reaction mixture was poured into ice water and extracted with methylene chloride. The organic layer was washed with sat. brine, dried over sodium sulfate, filtered and concentrated under reduced pressure. Column chromatography (SiO₂, 10% methanol/90% methylene chloride) yielded the envisaged tetrazole A-05 (10 mg, 13%) as a colorless solid.

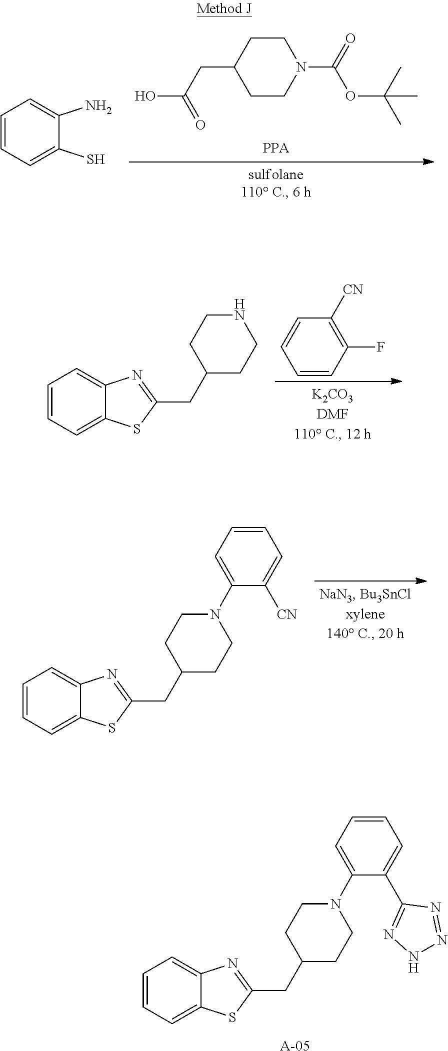

To a stirred solution of 2-aminobenzenethiol (0.5 g, 4.0 mmol) in sulfolane (10 mL) was added a piperidine derivative (1.16 g, 4.8 mmol) followed by PPA (2.5 mL, 16.0 mmol) after which the tube was sealed and the resultant mixture was stirred at 110° C. for 6 h. After the completion of the reaction was confirmed by TLC, the reaction mixture was basified with a 10% NaOH solution and extracted with CH₂Cl₂ (2×50 mL). The combined organic layers were washed with sat. brine, dried over anhydrous sodium sulfate and evaporated under reduced pressure. The crude compound thus obtained was purified by column chromatography over silica gel, eluting with 20-30% of EtOAc in hexane, affording 2-(4-piperidylmethyl)-1,3-benzothiazole as a colorless solid (710 mg, 77%).

To a stirred solution of 2-(4-piperidylmethyl)-1,3-benzothiazole (700 mg, 3.0 mmol) in DMF (20 mL) was added K₂CO₃ (1.25 g, 9.0 mmol), after which the reaction was kept stirring at r.t. for 15 minutes. 2-Fluorobenzonitrile (0.55 g, 4.5 mmol) was added at r.t. and the reaction was continued at 110° C. for 12 h. After completion of the reaction was confirmed by TLC, the reaction mixture was diluted with cold water and extracted with CH₂Cl₂ (3×50 mL). The combined organic layers were washed with sat. brine, dried over anhydrous sodium sulfate and evaporated under reduced pressure. The crude thus obtained was purified by column chromatography (silica gel, ₄₀-50% EtOAc in hexane) to afford the desired nitrile as a colorless solid (405 mg, 40%).

To a stirred solution of nitrile (70 mg, 0.21 mmol) in xylene (5 mL) in a sealed tube was added in NaN₃ (100 mg, 1.7 mmol) and Bu₃SnCl (0.45 mL, 1.7 mmol). The resultant mixture was stirred at 140° C. for 20 h. After completion of the reaction was confirmed by TLC, the reaction mixture was evaporated to dryness, the residue thus obtained was dissolved in CH₂Cl₂ and washed with a 10% NaOH solution. The aqueous layer was then neutralized with a citric acid solution and extraction with CH₂Cl₂ (2×25 mL) performed. The combined organic layers were washed with sat. brine, dried over anhydrous sodium sulfate and evaporated under reduced pressure to afford the crude residue. The crude compound was further purified by column chromatography over silica gel, eluting with 4-5% of MeOH in CH₂Cl₂, followed by trituration with diethyl ether to afford A-06 as a colorless solid (41 mg, 51%).

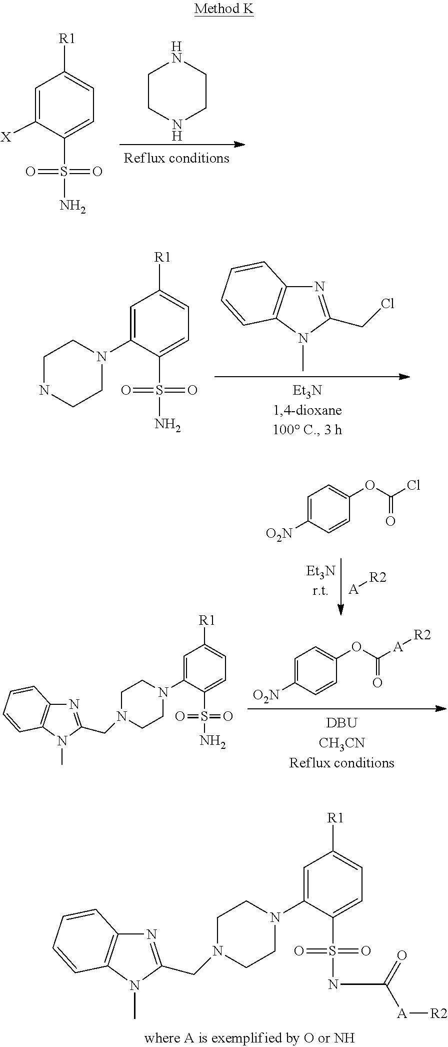

To a stirred solution of a substituted phenylsulfonamide of interest (2.65 mmol) in CH₃CN (10 mL) was added piperazine (6.6 mmol), after which the reaction mixture was refluxed at 80° C. until completion of the reaction by TLC was observed. The reaction mixture was evaporated in vacuo and the crude product obtained was purified by column chromatography over silica gel (2-4% MeOH in CH₂Cl₂) to afford the SNAr product.

To an ice-cold solution of the purified sulfonamide (1.57 mmol) in 1,4-dioxane (10 mL) was added triethylamine (0.66 mL, 4.71 mmol), followed by 2-(chloromethyl)-1-methyl-1H-benzo[d]imidazole (0.425 g, 2.355 mmol). The reaction mixture was stirred at 100° C., while monitoring the completion of the reaction by TLC. Upon completion, the reaction mixture was evaporated to dryness, diluted with ethyl acetate, washed with water and sat. brine, dried over anhydrous sodium sulfate and evaporated under reduced pressure to afford a crude residue. The residual solid was purified by column chromatography (silica gel, 45-50% ethyl acetate in hexane) to afford the alkylated piperazine derivative.

To an ice-cold solution of (4-nitrophenyl) chloroformate (0.5 g, 2.48 mmol) in CH₂Cl₂ or CH₃CN (20 mL) was added a primary alcohol or amine of choice (4.975 mmol), followed by triethylamine or DIPEA (6.2 mmol). The resulting solution was stirred at r.t. until completion of the reaction was observed by TLC. Next, the reaction mixture was evaporated under reduced pressure to afford the envisaged product which was used as such in the next step without further purification.

To a stirred solution of alkylated sulfonamide (0.25 mmol) in CH₃CN (5 mL) was added the carbamate or carbonate of choice (0.375 mmol), followed by DBU (57 mg, 0.375 mmol). The resulting solution was refluxed until completion of the reaction by TLC was reached. Next, the reaction mixture was evaporated to dryness, diluted in ethyl acetate, washed with water, sat. brine, dried over anhydrous sodium sulfate and evaporated under reduced pressure. The crude residue thus obtained was purified by column chromatography over silica gel.

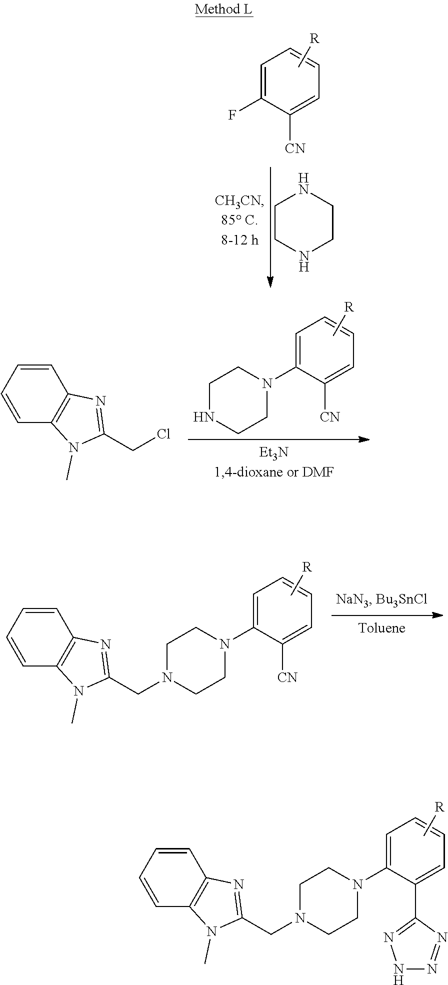

To a stirred solution of a substituted 2-fluorobenzonitrile (1 equiv.) in acetonitrile at 0° C. was added piperazine (5 equiv.) after which the resulting solution was stirred at 85° C. for 8-12 h. After completion of the reaction was confirmed by TLC, the reaction mixture was evaporated in vacuo, water was added, followed by extraction with EtOAc. The combined organic layers were washed with sat. brine, dried over anhydrous sodium sulfate and evaporated under reduced pressure. The crude obtained was taken to the next step without further purification.

To a stirred solution of substituted 2-(piperazin-1-yl)benzonitrile (1.0 mmol) in 1,4-dioxane or DMF, triethylamine (3.0 mmol) was added at r.t., followed after 10 minutes by addition of 2-(chloromethyl)-1-methyl-1H-benzo[d]imidazole (1.1 mmol). The reaction mixture was stirred at r.t. or 100° C. for 4-6 h. After completion of the reaction was confirmed by TLC, water was added to the reaction mixture followed by extraction with ethyl acetate. The combined organic layers were washed with water, sat. brine, dried over anhydrous sodium sulfate and evaporated under reduced pressure. The crude residual compound was purified by column chromatography over silica gel, eluting with MeOH in CH₂Cl₂ to afford the envisaged compound.

A mixture of the nitrile containing intermediate (1 equiv.), NaN₃ (8 equiv.) and Bu₃SnCl (8 equiv.) in toluene was stirred at 150° C. for 18-24 h in a sealed tube. After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layer was washed with sat. brine, dried over anhydrous sodium sulfate and evaporated under reduced pressure. The crude compound was purified by trituration or column chromatography over silica gel.

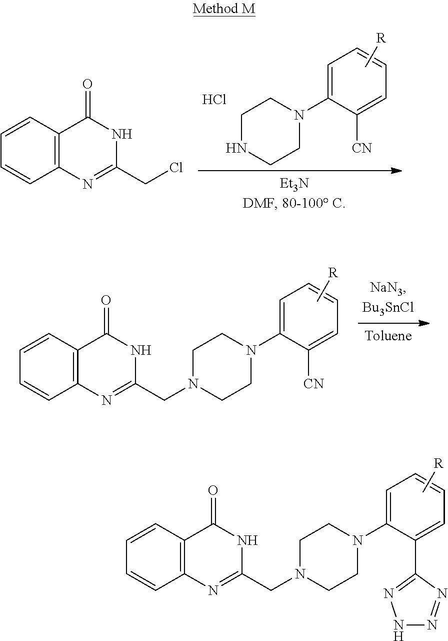

To a stirred solution of a substituted 2-(piperazin-1-yl)benzonitrile of interest (1 equiv.) in DMF, was added triethylamine (2.5-3 equiv.) at r.t., followed after 10 minutes by addition of 2-(chloromethyl)quinazolin-4-(3H)-one (1.2-1.5 equiv.). The reaction mixture was stirred at r.t. or 80-100° C. After completion of the reaction was confirmed by TLC, the reaction mixture was diluted with cold water and extracted with ethyl acetate. The combined organic layers were washed with water, sat. brine, dried over anhydrous sodium sulfate and evaporated under reduced pressure. The crude residual compound was purified by column chromatography over silica gel.

A mixture of the nitrile containing intermediate (1 equiv.), NaN₃ (5 to 8 equiv.) and Bu₃SnCl (5 to 8 equiv.) in toluene was stirred at 150° C. for 14-24 h in a sealed tube. After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layer was washed with sat. brine, dried over anhydrous sodium sulfate and evaporated under reduced pressure. The crude compound was purified by trituration or column chromatography over silica gel.

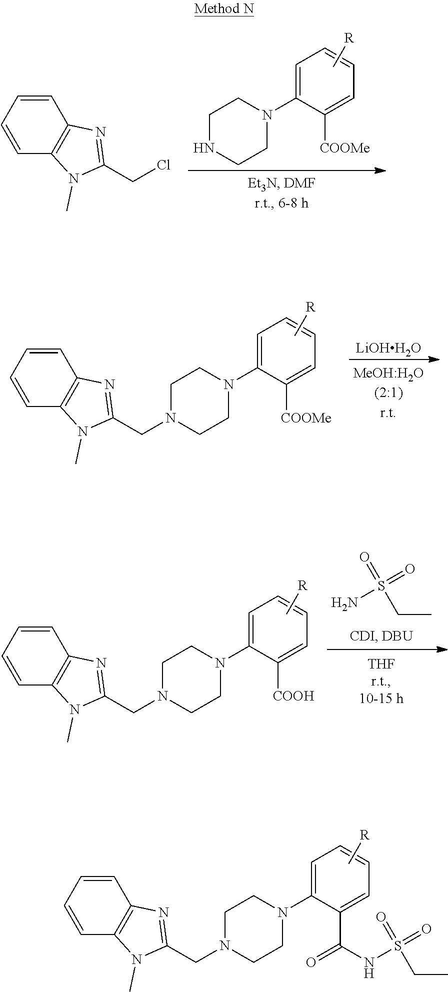

To a stirred solution of intermediate 12 (1.58 mmol) in DMF (20 mL) was added Et₃N (0.66 mL, 4.76 mmol) at 0° C., followed after 10 minutes by the addition of 2-(chloromethyl)-1-methyl-1H-benzo[d]imidazole (0.32 g, 1.74 mmol). The resulting reaction mixture was stirred at r.t. for 6-8 h. After completion of the reaction was confirmed by TLC, the reaction mixture was diluted with water and extraction with ethyl acetate was performed.

The combined organic layers were washed with water, sat. brine, dried over anhydrous sodium sulfate and evaporated in vacuo to afford a crude residue. The crude compound was further purified by column chromatography over silica gel (20-30% EtOAc in hexane) to afford the envisaged nucleophilic substitution product.

A solution of the obtained ester (0.52 mmol) and lithium hydroxide monohydrate (25 mg, 1.04 mmol) in MeOH:H₂O (2:1) was stirred at room temperature. After completion of the reaction was confirmed by TLC and LC-MS, the reaction mixture was concentrated under reduced pressure to remove the volatiles and the residual solid was diluted with water. Extraction with ethyl acetate was performed, after which the aqueous phase was neutralized with citric acid. The carboxylic acid was extracted into ethyl acetate, after which the organic layer was dried over anhydrous Na₂SO₄, filtered and concentrated in vacuo to obtain the crude carboxylic acid of interest. The acid was used as such in the next step without additional purification.

To a solution of substituted 2-(4-((1-methyl-1H-benzo[d]imidazol-2-yl)methyl)piperazin-1-yl)benzoic acid (0.42 mmol) in anhydrous THE (10 mL) at 0° C. was added CDI (139 mg, 0.85 mmol), followed by DBU (0.13 mL, 0.85 mmol) and ethanesulfonamide (46 mg, 0.47 mmol) after which the solution was stirred at room temperature for 10-15 h. Upon completion of the reaction, as determined by TLC, the mixture was evaporated to dryness. The obtained residue was diluted with water and extraction with ethyl acetate was performed. The combined organic layers were washed with sat. brine, dried over anhydrous sodium sulfate and evaporated under reduced pressure. The crude solid was further purified by column chromatography over silica gel (2-5% MeOH in CH₂Cl₂).

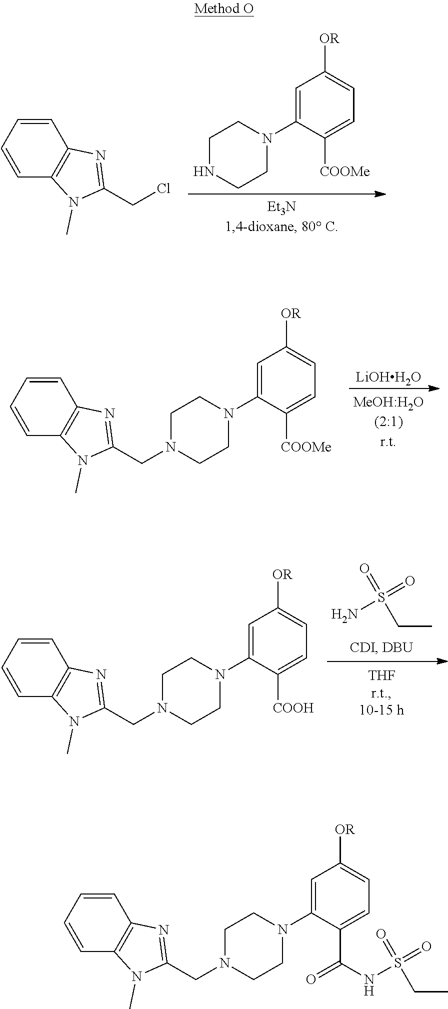

To a stirred solution of intermediate 13 (1.79 mmol) in 1,4-dioxane (30 mL) was added Et₃N (0.62 mL, 4.49 mmol) at 0° C., followed after 10 minutes by the addition of 2-(chloromethyl)-1-methyl-1H-benzo[d]imidazole (0.39 g, 2.15 mmol). The resulting reaction mixture was stirred at 80° C. After completion of the reaction was confirmed by TLC, the reaction mixture was diluted with water and extraction with ethyl acetate was performed. The combined organic layers were washed with water, sat. brine, dried over anhydrous sodium sulfate and evaporated in vacuo to afford a crude residue. The crude compound was further purified by column chromatography over silica gel.

A solution of the obtained ester (0.88 mmol) and lithium hydroxide monohydrate (42 mg, 1.75 mmol) in MeOH:H₂O (2:1) was stirred at room temperature until completion of the reaction was confirmed by TLC and LC-MS. The reaction mixture was concentrated under reduced pressure to remove the volatiles and the residual solid was diluted with water. Extraction with ethyl acetate was performed, after which the aqueous phase was neutralized with citric acid. The carboxylic acid was extracted into ethyl acetate, after which the organic layer was dried over anhydrous Na₂SO₄, filtered and concentrated in vacuo to obtain the crude compound of interest. The acid was used as such in the next step without additional purification.

To a solution of 4-alkoxy-2-(4-((1-methyl-1H-benzo[d]imidazol-2-yl)methyl)piperazin-1-yl)benzoic acid (0.24 mmol) in anhydrous THE (5 mL) at 0° C. was added CDI (79 mg, 0.49 mmol), followed by DBU (0.074 mL, 0.49 mmol) and ethanesulfonamide (32 mg, 0.29 mmol) after which the solution was stirred at room temperature. Upon completion of the reaction, as determined by TLC, the mixture was evaporated to dryness. The obtained residue was diluted with water and extraction with ethyl acetate was performed. The combined organic layers were washed with sat. brine, dried over anhydrous sodium sulfate and evaporated under reduced pressure. The crude solid was further purified by column chromatography over silica gel (5-10% MeOH in CH₂Cl₂).

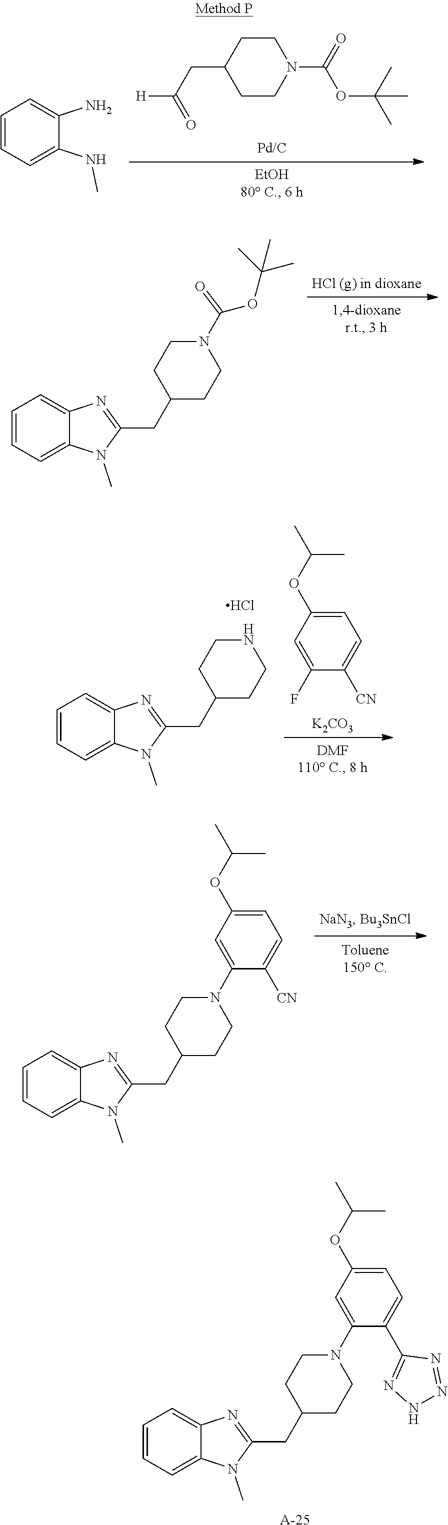

To a stirred solution of N-2-methylbenzene-1,2-diamine (0.5 g, 4.09 mmol) and tert-butyl 4-(2-oxoethyl)piperidine-1-carboxylate (0.93 g, 4.09 mmol) in EtOH (30 mL) was added Pd/C (100 mg) after which the reaction mixture was stirred at 80° C. for 6 h. After the completion of the reaction was confirmed by TLC, the reaction mixture was filtered through a Celite® bed and evaporated to dryness. Water was added to the obtained residue followed by extraction of the solution with ethyl acetate. The combined organic layers were washed with water, sat. brine, dried over anhydrous sodium sulfate and evaporated under reduced pressure to afford a crude mixture, which was further purified through column chromatography (SiO₂, 35-45% EtOAc in hexane), affording the desired scaffold as a pale-yellow solid (0.62 g, 46%).

To a stirred solution of tert-butyl 4-((1-methyl-1H-benzo[d]imidazol-2-yl)methyl)piperidine-1-carboxylate (0.5 g, 1.52 mmol) in 1,4-dioxane (5 mL) was added a solution of HCl (g) in dioxane (20 mL) at 0° C. Upon completion of the addition, the solution was left stirring at r.t. for 3 h. After completion of the reaction was confirmed by TLC, the reaction mixture was evaporated to dryness under reduced pressure and subsequently washed with hexane to afford the crude hydrochloride salt, which was taken to the next step without purification (0.36 g crude).

To a stirred solution of 1-methyl-2-(piperidin-4-ylmethyl)-1H-benzo[d]imidazole hydrochloride (100 mg, 0.38 mmol) in DMF (10 mL) was added K₂CO₃ (156 mg, 1.12 mmol), after which the reaction was kept stirring at r.t. for 10 minutes. 2-Fluoro-4-isopropoxybenzonitrile (80 mg, 0.45 mmol) was added at r.t. and the reaction was continued at 100° C. for 8 h. After completion of the reaction was confirmed by TLC, the reaction mixture was diluted with cold water and extracted with EtOAc. The combined organic layers were washed with sat. brine, dried over anhydrous sodium sulfate and evaporated in vacuo. The crude thus obtained was purified by column chromatography (silica gel, ₂-3% MeOH in CH₂Cl₂) to afford the desired nitrile as an off-white solid (90 mg, 61%).

A solution of nitrile (85 mg, 0.22 mmol), NaN₃ (114 mg, 1.75 mmol) and Bu₃SnCl (0.44 mL, 1.75 mmol) in toluene (5 mL) in a sealed tube was stirred at 150° C. for 24 h. After completion of the reaction was confirmed by TLC, the reaction mixture was evaporated under reduced pressure. The residual solids were dissolved in CH₂Cl₂ and washed with a 10% NaOH solution. The aqueous layer was then neutralized with a citric acid solution and extraction with CH₂Cl₂ was performed. The combined organic layers were washed with sat. brine, dried over anhydrous sodium sulfate and evaporated under reduced pressure to afford the crude residue. The crude compound thus obtained was further purified by column chromatography over silica gel, eluting with 5-6% of MeOH in CH₂Cl₂ to afford A-25 as an off-white solid (7 mg, 7%).

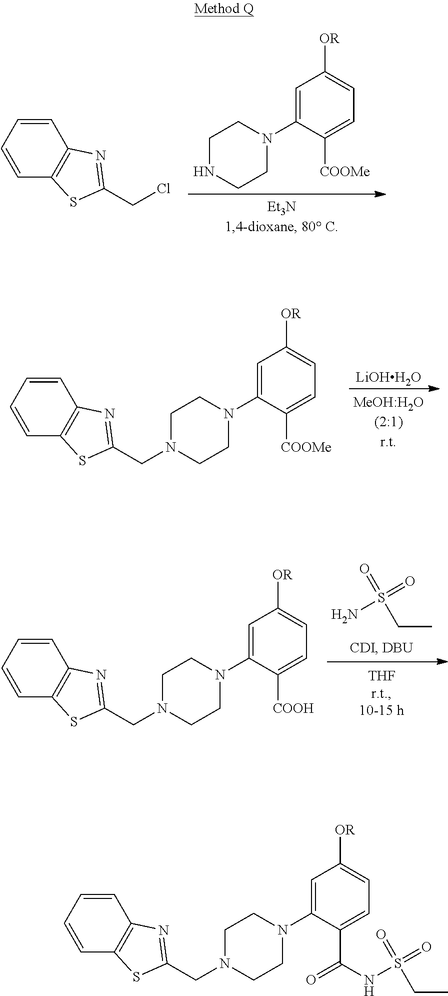

To a stirred solution of intermediate 13 (1.79 mmol) in 1,4-dioxane (30 mL) was added Et₃N (0.62 mL, 4.49 mmol) at 0° C., followed after 10 minutes by the addition of 2-(chloromethyl)benzo[d]thiazole (0.39 g, 2.15 mmol). The resulting reaction mixture was stirred at 80° C. After completion of the reaction was confirmed by TLC, the reaction mixture was diluted with water and extraction with ethyl acetate was performed. The combined organic layers were washed with water, sat. brine, dried over anhydrous sodium sulfate and evaporated in vacuo to afford a crude residue. The crude compound was further purified by column chromatography over silica gel.

A solution of the obtained ester (0.70 mmol) and lithium hydroxide monohydrate (34 mg, 1.41 mmol) in MeOH:H₂O (2:1) was stirred at room temperature until completion of the reaction was confirmed by TLC and LC-MS. The reaction mixture was concentrated under reduced pressure to remove the volatiles and water was added to the residual solid. Extraction with ethyl acetate was performed, after which the aqueous phase was neutralized with citric acid. The carboxylic acid was extracted into ethyl acetate, after which the organic layer was dried over anhydrous Na₂SO₄, filtered and concentrated in vacuo to obtain the crude compound of interest. The acid was used as such in the next step without additional purification.

To a solution of 4-alkoxy-2-(4-(benzo[d]thiazol-2-ylmethyl)piperazin-1-yl)benzoic acid (0.36 mmol) in anhydrous THE (8 mL) at 0° C. was added CDI (118 mg, 0.73 mmol), followed by DBU (0.10 mL, 0.73 mmol) and ethanesulfonamide (48 mg, 0.43 mmol) after which the solution was stirred at room temperature. Upon completion of the reaction, as determined by TLC, the mixture was evaporated to dryness. Water was added to the obtained residue followed by extraction of the reaction mixture with ethyl acetate. The combined organic layers were washed with sat. brine, dried over anhydrous sodium sulfate and evaporated under reduced pressure. The obtained crude solid was further purified by column chromatography over silica gel (5-10% MeOH in CH₂Cl₂).

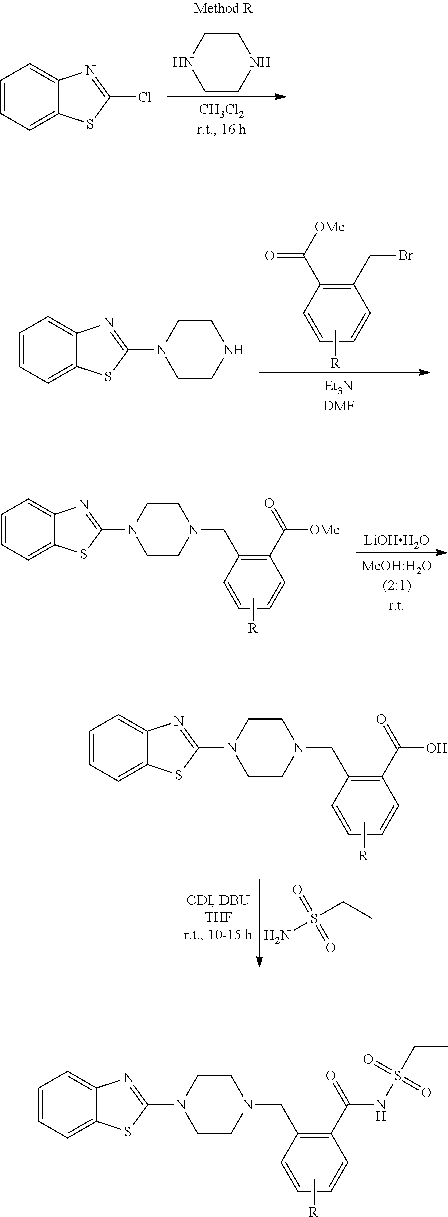

To a solution of 2-chloro-1,3-benzothiazole (2.0 g, 11.80 mmol) in dry methylene chloride (20 mL) was added piperazine (6.1 g, 71.00 mmol) and the solution was stirred at room temperature for 16 h. After this time, the reaction mixture was poured into ice water and extracted with methylene chloride. The organic layer was washed with sat. brine, dried over sodium sulfate, filtered and concentrated under reduced pressure to give the desired compound (2.5 g, 96%) as a colorless solid which was directly used in the next step without any further purification.

To a solution of 2-piperazin-1-yl-1,3-benzothiazole (0.23 g, 1.04 mmol) in anhydrous DMF (30 mL) at 0° C. was added triethylamine (0.36 mL, 2.70 mmol) and a substituted methyl 2-(bromomethyl)benzoate of interest (0.90 mmol), after which the reaction was kept stirring at room temperature or 100° C. Upon completion of the reaction as determined by TLC, the reaction mixture was poured into ice water and extracted with ethyl acetate. The organic layer was washed with sat. brine, dried over sodium sulfate, filtered and concentrated in vacuo to give a residue which was purified on a column of silica gel (2-5% MeOH in CH₂Cl₂).

A solution of the obtained ester (0.35 mmol) and lithium hydroxide monohydrate (17 mg, 0.70 mmol) in MeOH:H₂O (2:1) was stirred at room temperature until completion of the reaction was confirmed by TLC and LC-MS. The reaction mixture was concentrated under reduced pressure to remove the volatiles and water was added to the residual solids. Extraction with ethyl acetate was performed, after which the aqueous phase was neutralized with citric acid. The carboxylic acid was extracted into ethyl acetate, after which the organic layer was dried over anhydrous Na₂SO₄, filtered and concentrated in vacuo to obtain the crude compound of interest. The acid was used as such in the next step without additional purification.

To a solution of a substituted 2-((4-(benzo[d]thiazol-2-yl)piperazin-1-yl)methyl)benzoic acid (0.24 mmol) in dry THF (5 mL) at 0° C. was added CDI (79 mg, 0.48 mmol), followed by DBU (0.07 mL, 0.48 mmol) and ethanesulfonamide (32 mg, 0.29 mmol) after which the solution was stirred at room temperature for 10-15 h. Upon completion of the reaction, as determined by TLC, the mixture was evaporated to dryness. Water was added to the obtained residue followed by extraction with ethyl acetate. The combined organic layers were washed with sat. brine, dried over anhydrous sodium sulfate and concentrated under reduced pressure. The obtained crude solid was further purified by column chromatography over silica gel (5-10% MeOH in CH₂Cl₂).

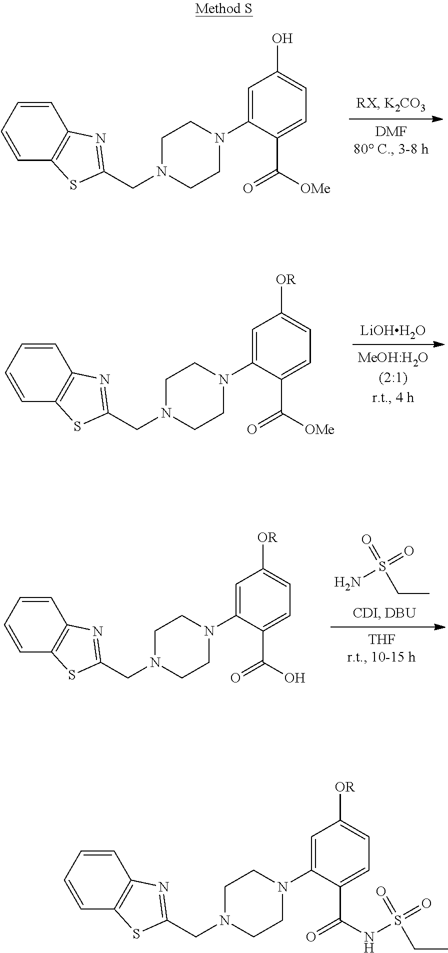

To a stirred solution of methyl 2-(4-(benzo[d]thiazol-2-ylmethyl)piperazin-1-yl)-4-hydroxybenzoate (200 mg, 0.52 mmol) in DMF (10 mL) was added K₂CO₃ (144 mg, 1.04 mmol) at 0° C., after which the solution was allowed to warm up to room temperature. After stirring at r.t. for 10 minutes, an alkyl halide of interest (0.78 mmol) was added, after which the reaction mixture was brought to 80° C. and kept stirring at this temperature for 3-8 h. After completion of the reaction was confirmed by TLC, water was added to the reaction mixture followed by extraction with ethyl acetate. The combined organic layers were washed with water, sat. brine, dried over anhydrous sodium sulfate and evaporated under reduced pressure to afford a crude residue. The obtained residual compound was purified by column chromatography over silica gel, eluting with EtOAc in hexane, affording the methyl 4-alkoxy-2-(4-(benzo[d]thiazol-2-ylmethyl)piperazin-1-yl)benzoate.

A solution of the obtained ester (0.38 mmol) and lithium hydroxide monohydrate (18 mg, 0.75 mmol) in MeOH:H₂O (2:1) was stirred at room temperature for 4 h. After completion of the reaction was confirmed by TLC and LC-MS, the reaction mixture was concentrated under reduced pressure to remove the volatiles and the residual solid was diluted with water. Extraction with ethyl acetate was performed, after which the aqueous phase was neutralized with citric acid. The carboxylic acid was extracted into ethyl acetate, after which the organic layer was dried over anhydrous Na₂SO₄, filtered and concentrated in vacuo to obtain the crude carboxylic acid of interest. The acid was used as such in the next step without additional purification.

To a solution of substituted 2-(4-(benzo[d]thiazol-2-ylmethyl)piperazin-1-yl)benzoic acid (0.31 mmol) in anhydrous THE (10 mL) at 0° C. was added CDI (101 mg, 0.63 mmol), followed by DBU (0.095 mL, 0.63 mmol) and ethanesulfonamide (41 mg, 0.38 mmol), after which the solution was stirred at room temperature for 10-15 h. Upon completion of the reaction, as determined by TLC, the mixture was evaporated to dryness. Water was added to the obtained residue followed by extraction with ethyl acetate. The combined organic layers were washed with sat. brine, dried over anhydrous sodium sulfate and evaporated under reduced pressure. The crude solid was further purified by column chromatography over silica gel (5-10% MeOH in CH₂Cl₂).

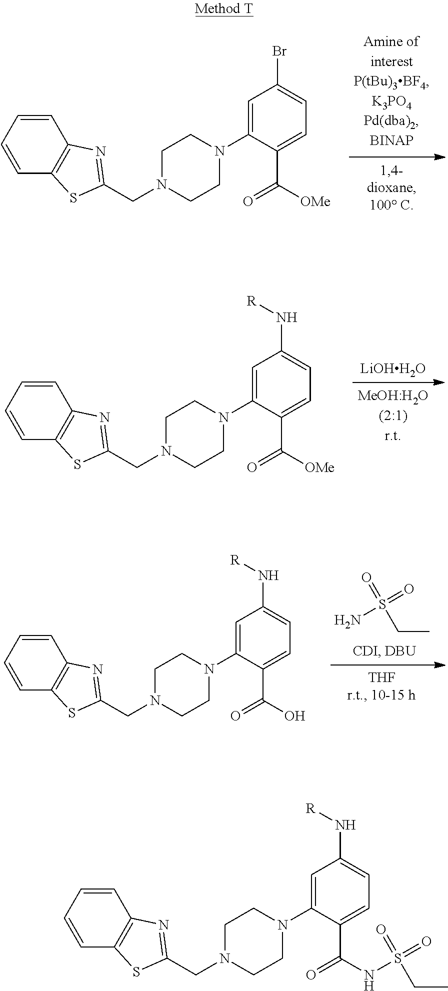

To a stirred solution of intermediate 3 (1 equiv.) in 1,4-dioxane was added a substituted amine of interest (1.5 equiv), followed by K₃PO₄ (2 equiv.) and P(tBu)₃·BF₄ (1 equiv.) and the resultant mixture was bubbled with argon for 20 min. Then Pd(dba)₂ (0.1 equiv.) and BINAP (0.2 equiv.) were added after which the reaction was heated to 100° C. until completion of the reaction was observed by TLC. The reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel yielded the desired compound.

Following the Buchwald coupling, a solution of substituted methyl 2-(4-(benzo[d]thiazol-2-ylmethyl)piperazin-1-yl)benzoate (1 equiv.) and lithium hydroxide monohydrate (2 equiv.) in MeOH:H₂O (2:1 ratio) was stirred at room temperature. After completion of the hydrolysis reaction, as determined by TLC and LC-MS, the mixture was concentrated under reduced pressure to remove the volatiles. Water was added to the organic residue and back washed with ethyl acetate. The aqueous phase was subsequently neutralized with 1M citric acid and extraction with ethyl acetate was performed. The combined organic layers were dried over anhydrous Na₂SO₄, filtered and concentrated under reduced pressure to afford the carboxylic acid which was used as such in the next step without any purification.

To a solution of the carboxylic acid (1 equiv.) in THE was added CDI (2 equiv.) at 0° C., after which the reaction was left stirring at room temperature for 15 minutes. Next, DBU (2 equiv.) and ethanesulfonamide (1.2 equiv.) were added, after which the reaction mixture was kept at room temperature overnight. After confirmation of the completion of the reaction by TLC, the solution was evaporated to dryness, water was added and the reaction mixture was extracted with ethyl acetate. The combined organic layers were washed with sat. brine, dried over anhydrous sodium sulfate and evaporated in vacuo. Finally, column chromatography over silica gel using a mixture of MeOH in CH₂Cl₂ allowed isolation of the envisaged compound.

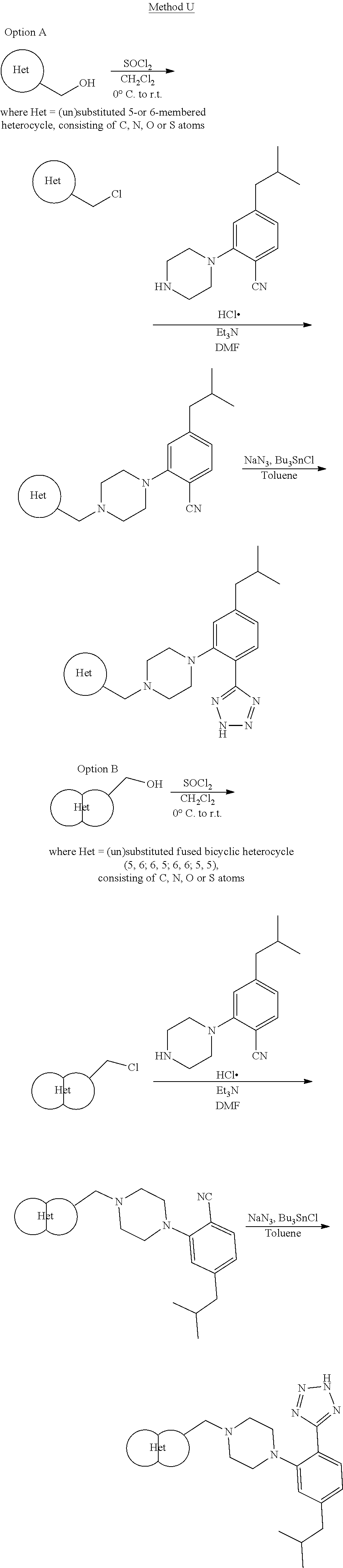

To a stirred solution of a 3-(hydroxymethyl) heterocycle (1 equiv.) in CH₂Cl₂ was added SOCl₂ (2 equiv.) at 0° C. and the reaction was allowed to stir for 1 to 3 hours. After completion of the reaction was confirmed by TLC, the reaction mixture was diluted with a cold sat. NaHCO₃ solution and extracted with CH₂Cl₂. The combined organic layers were washed with sat. brine, dried over anhydrous sodium sulfate and evaporated under reduced pressure. The obtained crude 3-(chloromethyl) heterocycle was used in the next step without further purification.

To a stirred solution of intermediate 1 (1 equiv.) in DMF at 0° C. was added triethylamine (3-4 equiv.) after which the reaction was stirred at room temperature for 10 minutes. Subsequently, a 3-(chloromethyl) heterocycle (1.2 equiv.) was added and the reaction was continued at room temperature or heated to 60-80° C. After completion of the reaction was confirmed by TLC, cold water was added to the reaction mixture which was followed by extraction with EtOAc. The combined organic layers were washed with sat. brine, dried over anhydrous sodium sulfate and evaporated under reduced pressure. The crude obtained was purified by column chromatography over silica gel.

A mixture of the nitrile containing intermediate (1 equiv.), NaN₃ (8 equiv.) and Bu₃SnCl (8 equiv.) in toluene was stirred at 140-150° C. in a sealed tube. After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The crude compound was purified through column chromatography over silica gel.

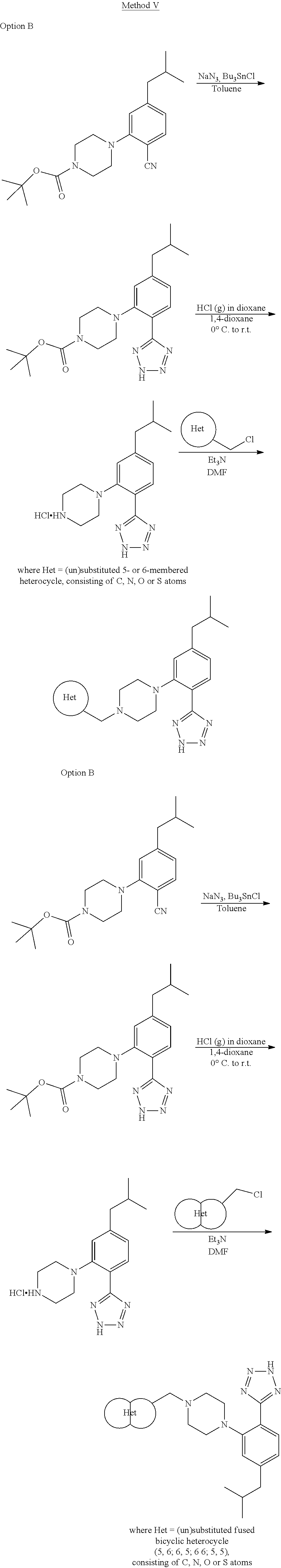

A mixture of Boc-protected 4-isobutyl-2-piperazin-1-ylbenzonitrile (1 equiv.), NaN₃ (8 equiv.) and Bu₃SnCl (8 equiv.) in toluene was stirred at 140-150° C. in a sealed tube.

After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The crude compound was purified through column chromatography over silica gel.

To a stirred solution of the Boc-protected tetrazole (1 equiv.) in 1,4-dioxane was added an excess of HCl (g) in dioxane at 0° C. The reaction was allowed to slowly warm up to room temperature and kept stirring at room temperature until complete conversion was confirmed by TLC. The reaction mixture was evaporated to dryness under reduced pressure, followed by trituration in an appropriate solvent (e.g. hexane). The obtained crude solid was used in the next step without further purification.

To a stirred solution of the tetrazole containing hydrochloride salt (1 equiv.) in DMF at 0° C. was added triethylamine (3-4 equiv.) after which the reaction was stirred at room temperature for 10 minutes. Subsequently, a 3-(chloromethyl) heterocycle (1.2 equiv.) was added and the reaction was continued at room temperature or heated to 60-80° C. After completion of the reaction was confirmed by TLC, the reaction mixture was diluted with cold water and extracted with EtOAc. The combined organic layers were washed with sat. brine, dried over anhydrous sodium sulfate and evaporated under reduced pressure. The crude obtained was purified by column chromatography over silica gel.

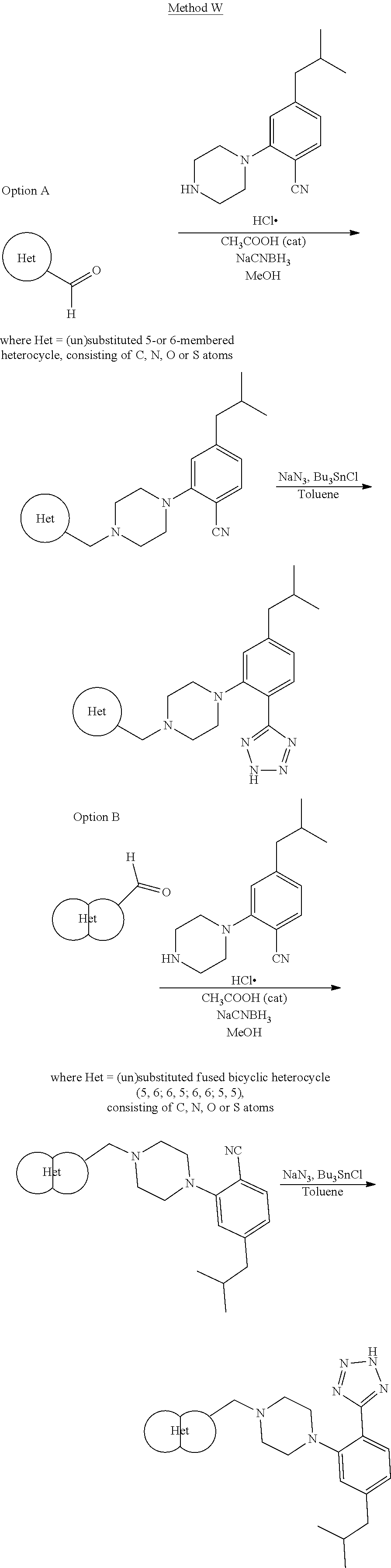

To a stirred solution of intermediate 1 (1 equiv.) in MeOH at 0° C. was added a heterocyclic aldehyde of interest (1.05 equiv.), followed by the addition of a catalytic amount of acetic acid, after which the reaction was stirred at room temperature until the imine formation was complete. Subsequently, sodium cyanoborohydride (3 equiv.) was added after which the reaction was continued at room temperature. After completion of the reaction was confirmed by TLC, cold water was added to the reaction mixture followed by extraction with EtOAc. The combined organic layers were washed with sat. brine, dried over anhydrous sodium sulfate and evaporated under reduced pressure. The obtained crude was purified by column chromatography over silica gel.

A mixture of the nitrile containing intermediate (1 equiv.), NaN₃ (8 equiv.) and Bu₃SnCl (8 equiv.) in toluene was stirred at 140-150° C. in a sealed tube. After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The crude compound was purified through column chromatography over silica gel.

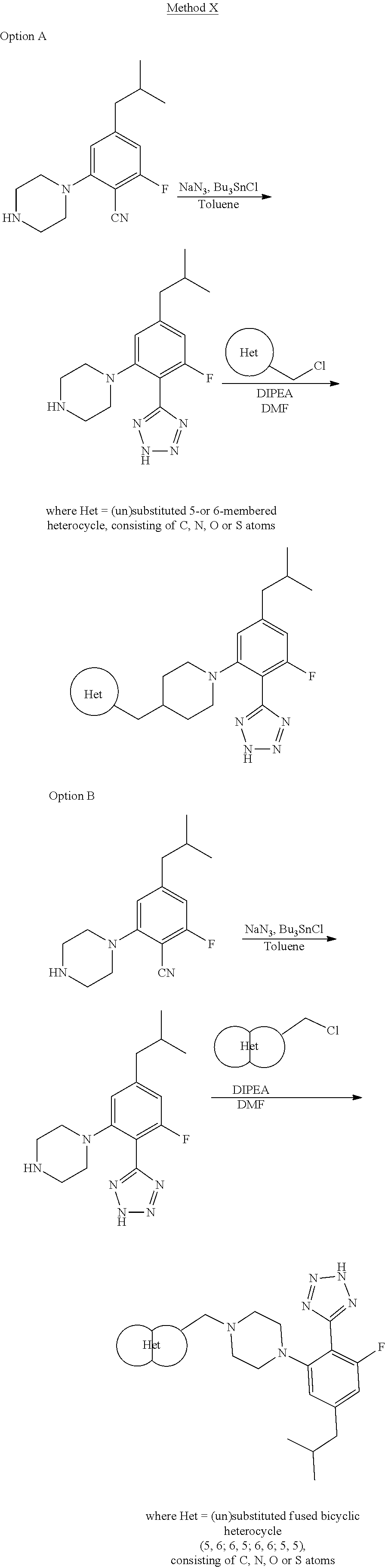

A mixture of intermediate 2 (1 equiv.), NaN₃ (8 equiv.) and Bu₃SnCl (8 equiv.) in toluene was stirred at 140-150° C. in a sealed tube. After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The crude compound was purified through column chromatography over silica gel.

To a stirred solution of the intermediate tetrazole (1 equiv.) in DMF at 0° C. was added DIPEA (2.5 equiv.) after which the reaction was stirred at room temperature for 10 minutes. Subsequently, a 3-(chloromethyl) heterocycle (1.2 equiv.) was added and the reaction was continued at room temperature or heated to 60-80° C. After completion of the reaction was confirmed by TLC, cold water was added to the reaction mixture followed by extraction with EtOAc. The combined organic layers were washed with sat. brine, dried over anhydrous sodium sulfate and evaporated under reduced pressure. The crude obtained was purified by column chromatography over silica gel.

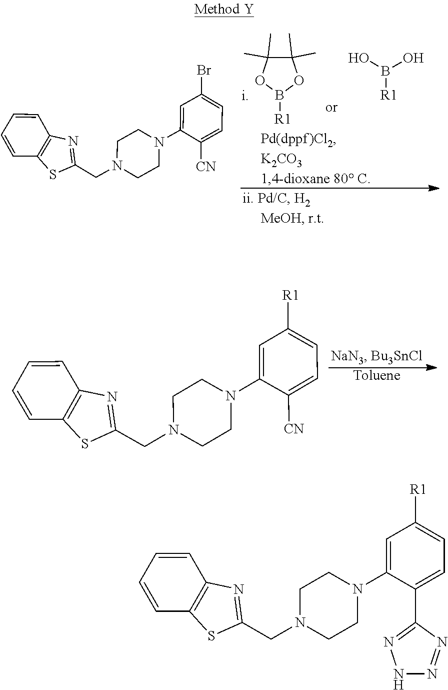

To a stirred solution of intermediate 19 (1 equiv.) in 1,4-dioxane was added a substituted boronic acid or boronic pinacol ester of interest (1.2 equiv), followed by K₂CO₃ (2.5 equiv.) and the resultant mixture was bubbled with argon for 20 min. Then Pd(dppf)Cl₂ (0.1 equiv.) was added after which the reaction was heated to 80° C. until completion of the reaction was observed by TLC. The reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel, yielded the desired compound.

When needed, a solution of a substituted 2-(4-(benzo[d]thiazol-2-ylmethyl)piperazin-1-yl)benzonitrile (1 equiv.) in MeOH was hydrogenated over 10% Pd/C under 5 Kg/cm² H₂ pressure using a Parr hydrogenator at ambient temperature. The reaction mixture was filtered over a Celite Bed®, optionally followed by a trituration in an appropriate solvent (e.g. diethyl ether).

Following the hydrogenation, a solution of substituted 2-(4-(benzo[d]thiazol-2-ylmethyl)piperazin-1-yl)benzonitrile (1 equiv.), NaN₃ (8 equiv.) and Bu₃SnCl (8 equiv.) in toluene was stirred at 140-150° C. in a sealed tube. After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layers were washed with sat. brine, dried over anhydrous sodium sulfate and evaporated in vacuo. The crude compound was purified through column chromatography over silica gel.

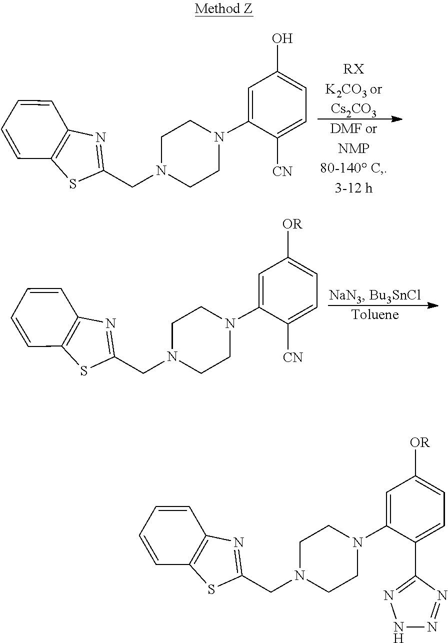

To a stirred solution of 2-(4-(benzo[d]thiazol-2-ylmethyl)piperazin-1-yl)-4-hydroxybenzonitrile (1 equiv., 250 mg, 0.71 mmol) in DMF or NMP (15 mL) was added K₂CO₃ or Cs₂CO₃ (2 equiv., 1.43 mmol) at 0° C., after which the solution was allowed to warm up to room temperature. After stirring at r.t. for 10 minutes, an alkyl halide of interest (1.2 equiv., 0.78 mmol) was added, after which the reaction mixture was brought to 80° C. (DMF) or 140° C. (NMP) and kept stirring at this temperature for 3-12 h. After completion of the reaction was confirmed by TLC, water was added to the reaction mixture followed by extraction with ethyl acetate. The combined organic layers were washed with water, sat. brine, dried over anhydrous sodium sulfate and evaporated under reduced pressure to afford a crude residue. The obtained residual compound was purified by column chromatography over silica gel.

Following the alkylation, a solution of substituted 2-(4-(benzo[d]thiazol-2-ylmethyl)piperazin-1-yl)benzonitrile (1 equiv.), NaN₃ (8 equiv.) and Bu₃SnCl (8 equiv.) in toluene was stirred at 140-150° C. in a sealed tube. After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layers were washed with sat. brine, dried over anhydrous sodium sulfate and evaporated in vacuo. The crude compound was purified through column chromatography over silica gel and triturated with an appropriate solvent of choice (e.g. diethyl ether).

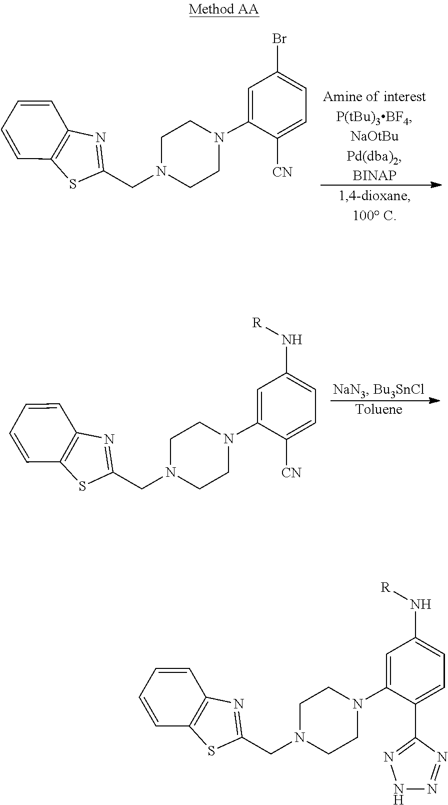

To a stirred solution of intermediate 19 (1 equiv.) in 1,4-dioxane was added a substituted amine of interest (1.5 equiv), followed by NaOtBu (1.5-2 equiv.) and P(tBu)₃·BF₄ (1.5-2 equiv.) and the resultant mixture was bubbled with argon for 20 min. Then Pd(dba)₂ (0.1 equiv.) and BINAP (0.2 equiv.) were added after which the reaction was heated to 100° C. until completion of the reaction was observed by TLC. The reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel yielded the desired substituted nitrile.

Following the Buchwald coupling, a solution of substituted 2-(4-(benzo[d]thiazol-2-ylmethyl)piperazin-1-yl)benzonitrile (1 equiv.), NaN₃ (8 equiv.) and Bu₃SnCl (8 equiv.) in toluene was stirred at 140-150° C. in a sealed tube. After completion of the reaction was confirmed by TLC, the reaction mixture was concentrated in vacuo and the thus obtained residue was re-dissolved in CH₂Cl₂. The organic layer was washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The crude compound was purified through column chromatography over silica gel and, when necessary, triturated with an appropriate solvent of choice (e.g. diethyl ether) to afford the envisaged substituted tetrazole.

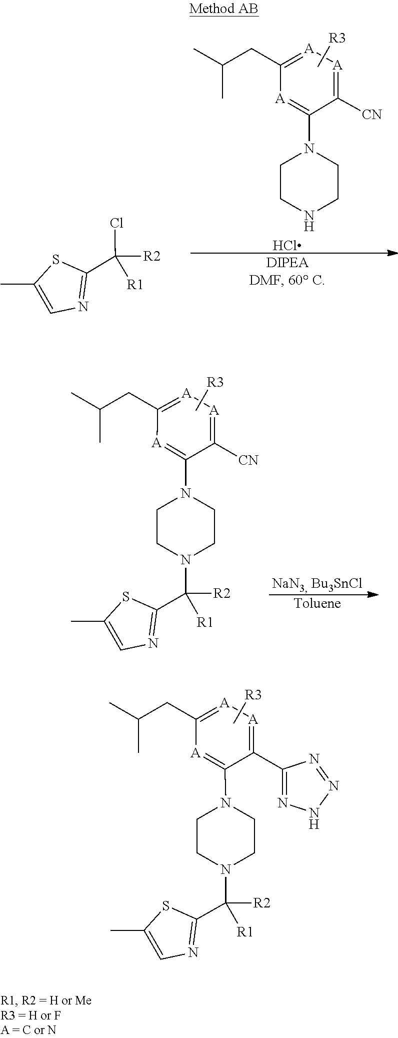

To a stirred solution of a substituted nitrile of interest (1 equiv.) in DMF at 0° C. was added DIPEA (2.5 equiv.) after which the solution was stirred at r.t. for 10 minutes. An optionally substituted 2-(chloromethyl)-5-methyl-thiazole (1.2 equiv.) was added and the reaction was continued at 60° C. for 8-12 hours. After completion of the reaction was confirmed by TLC, cold water was added and extraction with EtOAc was performed. The combined organic layers were washed with sat. brine, dried over anhydrous sodium sulfate and evaporated under reduced pressure. The crude thus obtained was purified by column chromatography over silica gel, eluting with 2-3% of MeOH in CH₂Cl₂.

A mixture of the obtained nitrile (1 equiv.), NaN₃ (5 to 8 equiv.) and Bu₃SnCl (5 to 8 equiv.) in toluene was stirred at 140-145° C. in a sealed tube until completion of the reaction was confirmed by TLC. The reaction mixture was evaporated under reduced pressure and the thus obtained residue was re-dissolved in CH₂Cl₂. The organic phase was washed with a 10% NaOH solution. The aqueous layer was then neutralized with a citric acid solution and extracted with CH₂Cl₂. The combined organic layers were washed with sat. brine, dried over anhydrous sodium sulfate and evaporated in vacuo. The crude compound was purified by silica chromatography, eluting the envisaged compound with 4-5% of MeOH in CH₂Cl₂.

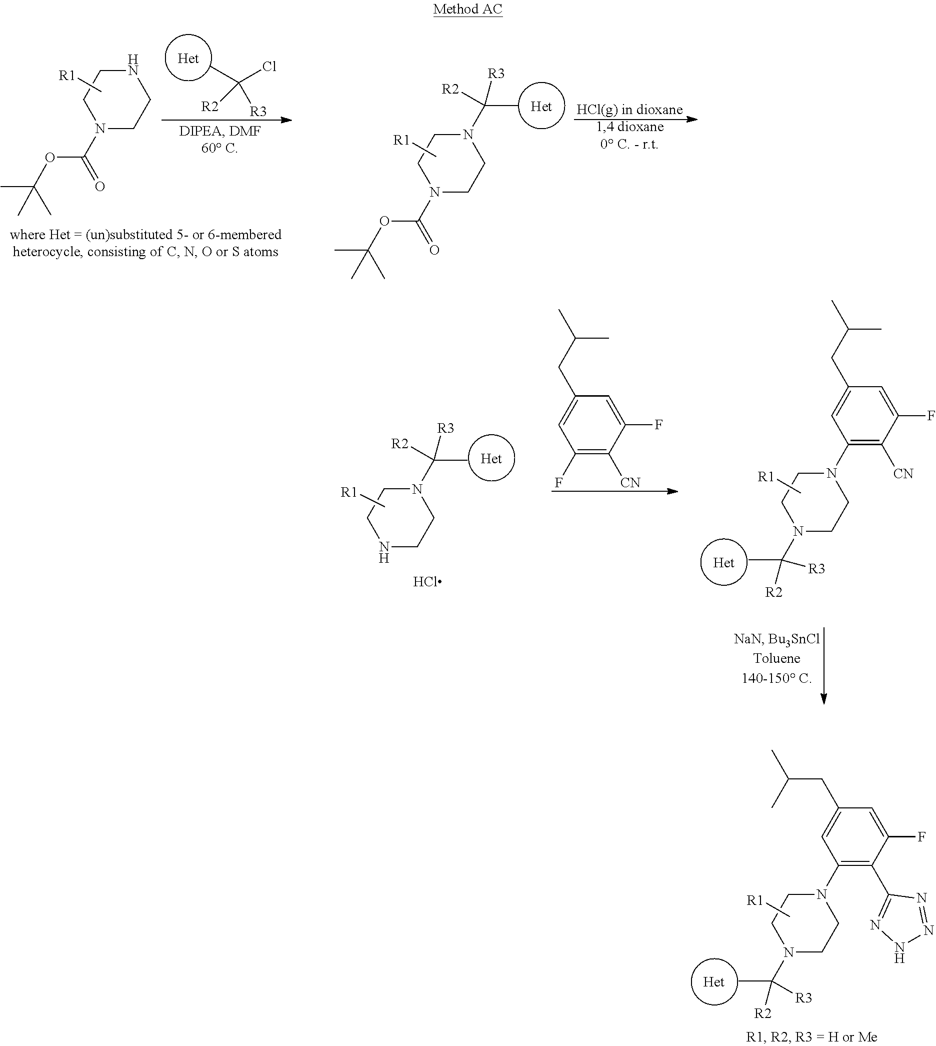

To a stirred solution of the substituted piperazine of interest (1 equiv.) in DMF at 0° C. was added DIPEA (2.5 equiv.) dropwise, after which the reaction was continued at room temperature for 10 minutes. Subsequently, a heteroaryl chloride (1.2 equiv.) was added after which the reaction was continued and brought to 60° C. when necessary to push conversion. After completion of the reaction was confirmed by TLC, the reaction mixture was extracted with cold water and EtOAc. The combined organic layers were washed with sat. brine, dried over anhydrous sodium sulfate and evaporated under reduced pressure. The crude obtained was purified by column chromatography over silica gel.

Following the alkylation reaction, the isolated compound (1 equiv.) was dissolved in 1,4-dioxane to which HCl (g) in dioxane was added at 0° C. Upon completion of the addition, the reaction was allowed to slowly warm up to room temperature and kept stirring until complete conversion was obtained (determined via TLC). The reaction mixture was concentrated in vacuo, followed by washing of the crude residue with hexane. The crude compound was taken to the next step without additional purification.

The isolated compound was taken into a SNAr reaction. Therefore, to a stirred solution of the deprotected compound (1.0 equiv.) in DMF at room temperature was added DIPEA (2.5 equiv.) and/or K₂CO₃ (2.0 equiv.). Subsequently, 2,6-difluoro-4-isobutylbenzonitrile (1.1 equiv.) was added to this mixture at room temperature, after which the reaction was brought to 65° C. When the completion of the reaction was confirmed by TLC, the solution was cooled down, cold water was added and an extraction with EtOAc was performed. The combined organic layers were washed with sat. brine, dried over anhydrous sodium sulfate and evaporated under reduced pressure. The organic residue was purified by column chromatography over silica gel.

In a final stage, a mixture of the nitrile containing intermediate (1 equiv.), NaN₃ (5 to 8 equiv.) and Bu₃SnCl (5 to 8 equiv.) in toluene was stirred at 140-150° C. in a sealed tube.

After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The crude compound was purified by column chromatography over silica gel.

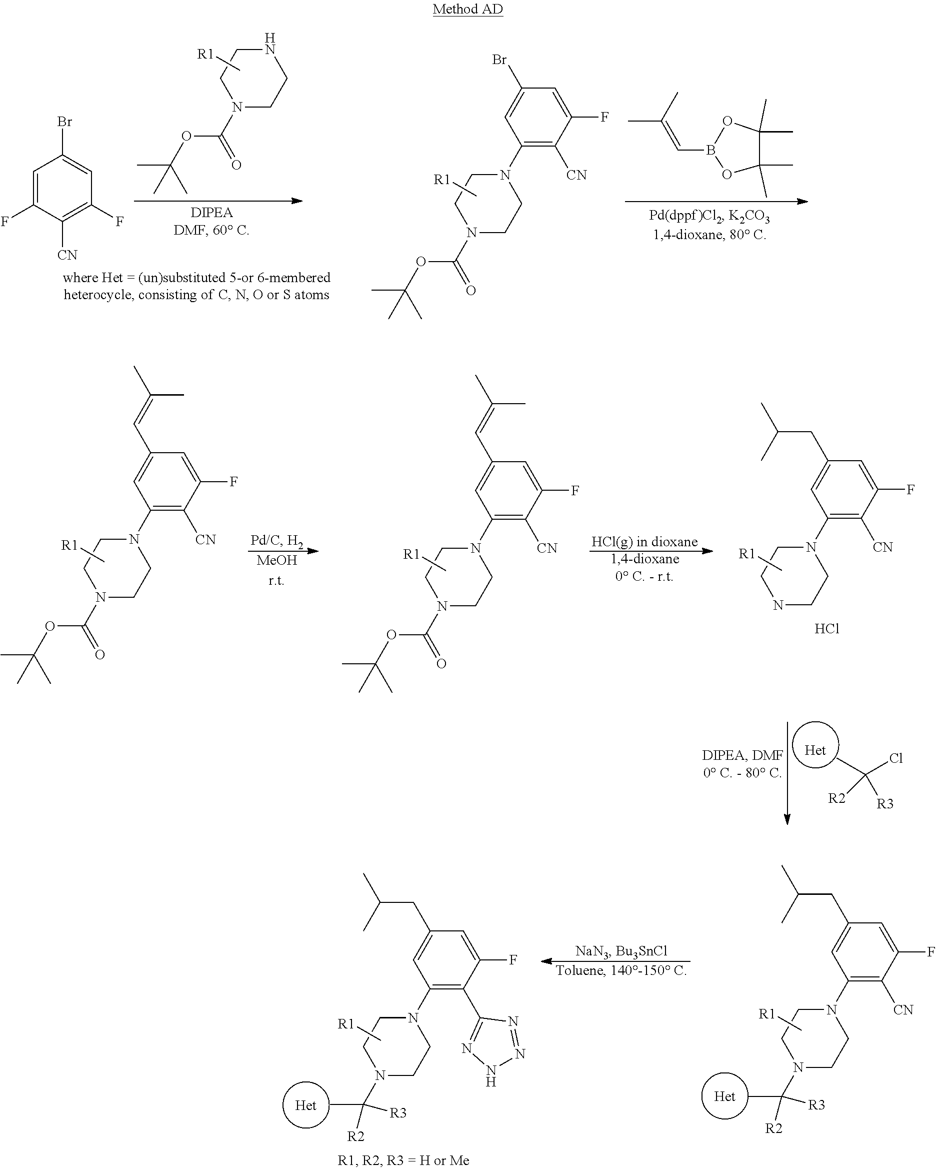

To a stirred solution at room temperature of a substituted piperazine of interest (1.2 equiv.) in DMF was added DIPEA (2.5 equiv.). Subsequently, 4-bromo-2,6-difluorobenzonitrile (1 equiv.) was added to this mixture at room temperature, after which the reaction was brought to 60° C. until completion of the reaction was confirmed by TLC. The solution was cooled down, cold water was added, followed by extraction with EtOAc. The combined organic layers were washed with sat. brine, dried over anhydrous sodium sulfate and evaporated under reduced pressure. The organic residue was purified by column chromatography over silica gel.

To a stirred solution of the isolated SNAr product (1 equiv.) in 1,4-dioxane was added 4,4,5,5-tetramethyl-2-(2-methylprop-1-en-1-yl)-1,3,2-dioxaborolane (1.2 equiv.), followed by K₂CO₃ (2.5 equiv.) and the resultant mixture was bubbled with argon for 20 min. Then Pd(dppf)Cl₂ (0.1 equiv.) was added after which the reaction was heated to 80° C. until completion of reaction was observed by TLC. The reaction mixture was concentrated under reduced pressure to remove the volatiles and the residue was re-dissolved with ethyl acetate and washed with water and sat. brine. Subsequent drying over anhydrous sodium sulfate and evaporation under reduced pressure, followed by column chromatography over silica gel, afforded the compound of interest.

Following the Suzuki coupling, a hydrogenation using a Parr hydrogenator at ambient temperature was performed. Therefore, the Suzuki product (1 equiv.) was dissolved in MeOH in the presence of 10% Pd/C while putting the reaction under 5 Kg/cm² H₂ pressure. After confirming the completion of the reaction by LC-MS, the reaction mixture was filtered through a Celite® bed and was concentrated in vacuo, yielding the envisaged hydrogenated compound. The crude compound was used as such in the following deprotection.

In a next step, the isolated compound (1 equiv.) was dissolved in 1,4-dioxane to which HCl (g) in dioxane was added at 0° C. Upon completion of the addition, the reaction was allowed to slowly warm up to room temperature and kept stirring until complete conversion was obtained (via TLC). The reaction mixture was concentrated in vacuo, followed by washing of the crude residue with hexane. The crude compound was taken to the next step without additional purification.

To a stirred solution of the deprotected compound (1 equiv.) in DMF at 0° C. was added DIPEA (3 equiv.) dropwise, after which the reaction was continued at room temperature for 10 minutes. Subsequently, a heteroaryl chloride of interest (1.2 equiv.) was added and the reaction was continued at room temperature or heated to 80° C. After completion of the reaction was confirmed by TLC, cold water was added to the reaction mixture, followed by extraction with CH₂Cl₂. The combined organic layers were washed with sat. brine, dried over anhydrous sodium sulfate and evaporated under reduced pressure. The crude obtained was purified by column chromatography over silica gel.

In a final reaction, a mixture of the nitrile containing intermediate (1 equiv.), NaN₃ (5 to 8 equiv.) and Bu₃SnCl (5 to 8 equiv.) in toluene was stirred at 140-150° C. in a sealed tube. After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layer was washed with sat. brine, dried over anhydrous sodium sulfate and evaporated under reduced pressure. The crude compound was purified by column chromatography over silica gel.

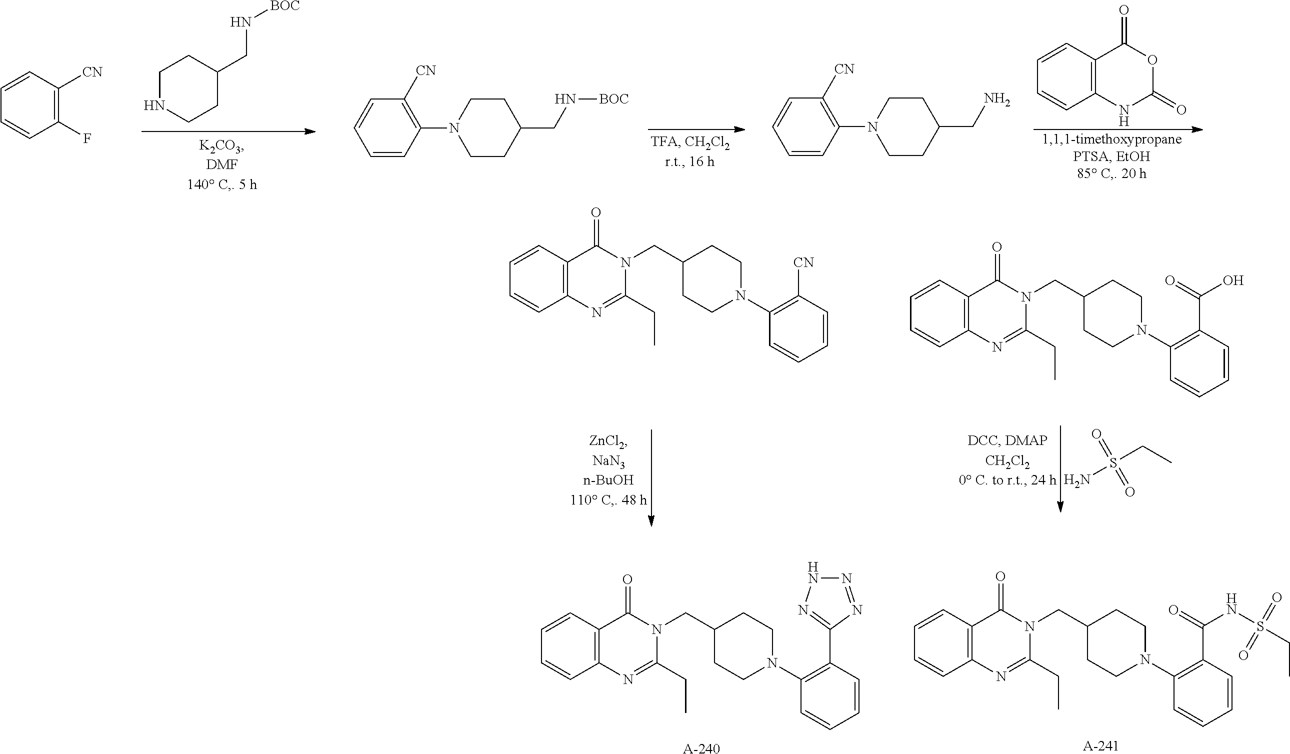

To a solution of 2-fluorobenzonitrile (2.00 g, 16.51 mmol) in dry DMF (15 mL) was added tert-butyl (piperidin-4-ylmethyl)carbamate (3.54 g, 16.51 mmol) and K₂CO₃ (4.56 g, 33.02 mmol) after which the solution was brought to 140° C. for 5 h. After this time, the reaction mixture was poured into ice water and extracted with methylene chloride. The organic layer was washed with sat. brine, dried over sodium sulfate, filtered and concentrated under reduced pressure. Subsequent column chromatography of the obtained crude residue on silica gel (15% EtOAc in hexane) afforded tert-butyl N-[[1-(2-cyanophenyl)-4-piperidyl]methyl]carbamate as a colorless solid (2.50 g, 48%).

In a next step, the isolated compound (1.00 g, 3.17 mmol) was dissolved in methylene chloride (10 mL) to which trifluoroacetic acid (1.00 mL, 12.7 mmol) was added at 0° C. Upon completion of the addition, the reaction was allowed to slowly warm up to room temperature and kept stirring at this temperature for 16 h. When complete conversion of the reaction was obtained (via TLC), the reaction mixture was concentrated in vacuo. The obtained residue was re-dissolved in methanol and the pH was adjusted to ˜10 using basic Amberlite™. The slurry was filtered and washed with methanol, followed by concentration of the filtrate under reduced pressure to deliver 2-[4-(aminomethyl)-1-piperidyl]benzonitrile (594 mg, 87%). The obtained nitrile was taken to the next step without additional purification.

To a solution of 2-[4-(aminomethyl)-1-piperidyl]benzonitrile (2.70 g, 12.54 mmol) in anhydrous EtOH (5 mL) was added 1H-3,1-benzoxazine-2,4-dione (2.05 g, 12.54 mmol) and PTSA monohydrate (239 mg, 1.25 mmol) after which the reaction was stirred at 85° C. for 2 h. At this point, 1,1,1-trimethoxypropane (15.0 mL, 100.3 mmol) was added and stirring was continued at 85° C. for an additional 18 hours. Upon completion of the reaction (as determined by TLC), the reaction mixture was cooled down to 0° C. The resulting precipitate was isolated to yield 2-[4-[(2-ethyl-4-oxo-quinazolin-3-yl)methyl]-1-piperidyl]benzonitrile as a colorless solid (2.01 g, 43%).

A solution of 2-[4-[(2-ethyl-4-oxo-quinazolin-3-yl)methyl]-1-piperidyl]benzonitrile (450 mg, 1.21 mmol) in 80% sulfuric acid (5 mL) was stirred at 90° C. for 24 h, after which the reaction mixture was poured into ice water and the pH was adjusted to ˜5 with 2N aqueous sodium hydroxide. The aqueous phase was extracted with methylene chloride, washed with sat. brine, dried over sodium sulfate, filtered and concentrated under reduced pressure to give a residue which was purified using column chromatography (SiO₂, 20% EtOAc in hexane) to deliver the carboxylic acid of interest as a colorless solid (350 mg, 74%).

To a solution of 2-[4-[(2-ethyl-4-oxo-quinazolin-3-yl)methyl]-1-piperidyl]benzoic acid (75 mg, 0.19 mmol) in dry methylene chloride (5 mL) at r.t. was added DCC (395 mg, 1.92 mmol), followed by DMAP (234 mg, 1.9 mmol) and ethanesulfonamide (32 mg, 0.29 mmol) after which the solution was stirred at room temperature for 24 h. After this time, the reaction mixture was poured into ice water and extraction with methylene chloride was performed. The organic layer was washed with 10% aqueous citric acid, sat. brine, dried over sodium sulfate, filtered and concentrated under reduced pressure. The obtained residue was further purified on a column of silica gel (20% EtOAc in hexane), affording the envisaged acylsulfonamide A-241 as a colorless solid (31 mg, 33%).

To a solution 2-[4-[(2-ethyl-4-oxo-quinazolin-3-yl)methyl]-1-piperidyl]benzonitrile (300 mg, 0.81 mmol) in n-butanol (5 mL) was added NaN₃ (63 mg, 0.97 mmol) and ZnCl₂ (121 mg, 0.89 mmol) after which the reaction was brought to 110° C. for 2 days. After this time, the reaction mixture was poured into ice water and the reaction mixture was extracted with methylene chloride. The organic layer was washed with a 10% NaOH solution and subsequently, the aqueous layer was neutralized with a citric acid solution. Extraction of the aq. phase with CH₂Cl₂ was performed. The combined organic layers were washed with sat. brine, dried over anhydrous sodium sulfate and evaporated under reduced pressure to afford the crude residue, which was further purified through column chromatography over silica gel (20% EtOAc in hexane) to deliver the targeted tetrazole A-240 as a colorless solid (54 mg, 16%).

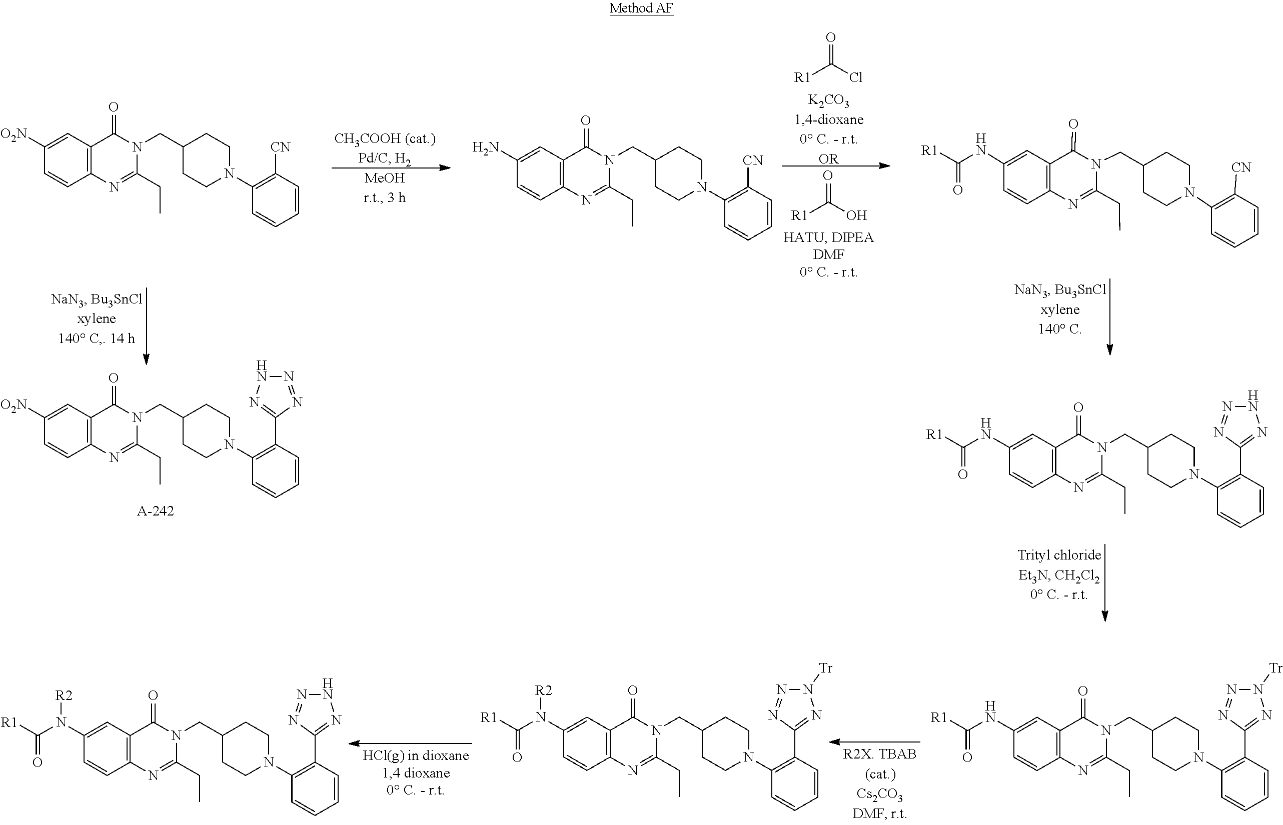

To a stirred solution of intermediate 59 (200 mg, 0.479 mmol) in anhydrous MeOH (20 mL) was added a catalytic amount of acetic acid and 10% Pd/C (50 mg) while putting the reaction under 5 Kg/cm² H₂ pressure using a Parr hydrogenator for 3 h at ambient temperature. After confirming the completion of the reaction by LC-MS, the reaction mixture was filtered through a Celite® bed and was concentrated in vacuo, yielding the envisaged hydrogenated compound which was used as such in the following amide coupling.

To a stirred solution of 2-[4-[(6-amino-2-ethyl-4-oxo-quinazolin-3-yl)methyl]-1-piperidyl]benzonitrile (185 mg, 0.478 mmol) in 1,4-dioxane (10 mL) at 0° C. was added K₂CO₃ (132 mg, 0.955 mmol), followed by an acyl chloride of interest (0.573 mmol). Alternatively, the amide coupling was performed by stirring a solution of 2-[4-[(6-amino-2-ethyl-4-oxo-quinazolin-3-yl)methyl]-1-piperidyl]benzonitrile (185 mg, 0.478 mmol) in DMF (5 mL) at 0° C. to which a carboxylic acid of interest (0.525 mmol), HATU (218 mg, 0.573 mmol) and DIPEA (0.21 mL, 1.19 mmol) were added. Upon completion of the addition, the reaction mixture was allowed to stir at r.t. for 14 h until complete conversion was observed by TLC. Water was added to the reaction mixture and extraction with EtOAc was performed. The combined organic layers were washed with sat. brine, dried over anhydrous sodium sulfate and evaporated under reduced pressure. The crude residue was purified by silica chromatography (3-5% of MeOH in CH₂Cl₂).

Next, a mixture of the nitrile containing intermediate (1 equiv.), NaN₃ (8 equiv.) and Bu₃SnCl (8 equiv.) in xylene was stirred at 140° C. in a sealed tube. After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The crude compound was purified by column chromatography (silica gel, 5-8% MeOH in CH₂Cl₂).

To a stirred solution of tetrazole of interest (1.85 mmol) in anhydrous CH₂Cl₂ at 0° C. was added Et₃N (0.39 mL, 2.77 mmol), followed by careful addition of trityl chloride (0.50 mL, 2.03 mmol). Upon completion of the addition, the reaction was allowed to slowly warm up to room temperature and was kept stirring at r.t. until completion of the reaction was confirmed by TLC. Water was added to the reaction mixture and extraction with CH₂Cl₂ was performed. The combined organic layers were washed with sat. brine, dried over anhydrous sodium sulfate and evaporated under reduced pressure, after which a trituration of the obtained crude residue with diethyl ether was performed.

To a stirred solution of an N-acyl-6-amino-2-ethyl-3-[[1-[2-(2-tritiotetrazol-5-yl)phenyl]-4-piperidyl]methyl]quinazolin-4-one of interest (0.125 mmol) in DMF (5 mL) was added Cs₂CO₃ (81 mg, 0.250 mmol), followed by the addition of an alkyl/aryl halide of interest (0.150 mmol) and a catalytic amount of TBAB, respectively, after which the reaction was continued stirring at r.t. for 14 h. After the completion of the reaction was confirmed by TLC, cold water was added to the reaction mixture and extraction with EtOAc was performed. The combined organic layers were washed with sat. brine, dried over anhydrous sodium sulfate and evaporated under reduced pressure. The obtained organic residue was triturated using diethyl ether to afford the envisaged alkylated intermediate.

In a final step, the alkylated intermediate (1 equiv., 0.105 mmol) was dissolved in 1,4-dioxane to which HCl (g) in dioxane (5 mL) was added at 0° C. Upon completion of the addition, the reaction was allowed to slowly warm up to room temperature and kept stirring at this temperature until complete conversion was obtained (via TLC). The reaction mixture was concentrated in vacuo, followed by a trituration with an appropriate solvent of choice (e.g. diethyl ether).

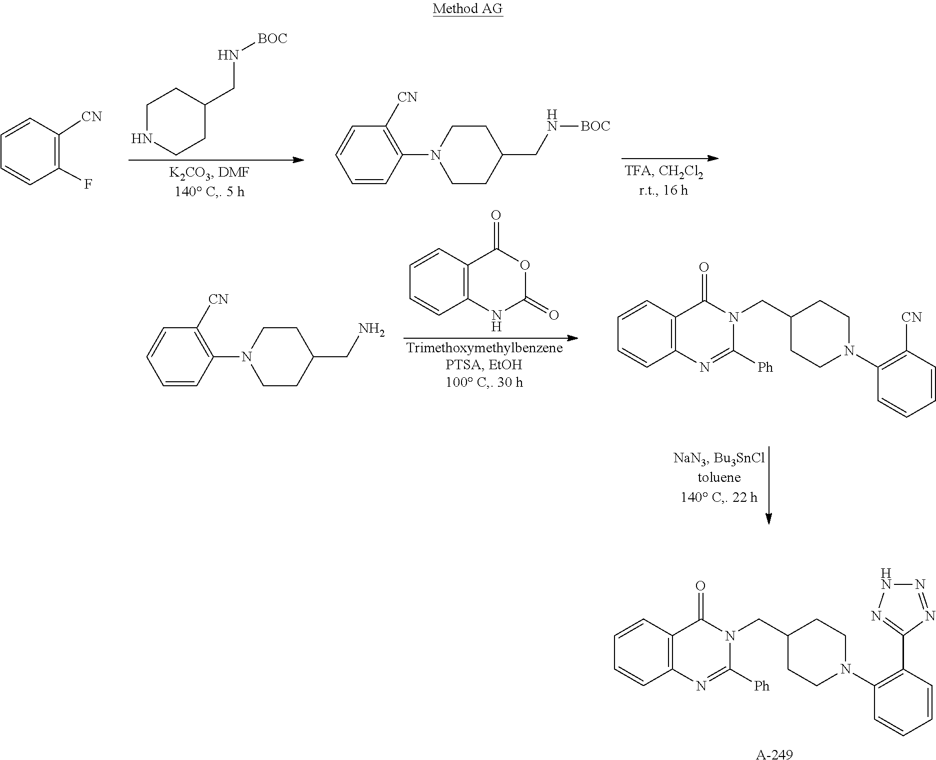

To a stirred solution of tert-butyl (piperidin-4-ylmethyl)carbamate (2.12 g, 9.91 mmol) and K₂CO₃ (2.28 g, 16.5 mmol) in dry DMF (25 mL) at r.t. was added 2-fluorobenzonitrile (1.00 g, 8.26 mmol) after which the reaction mixture was brought to 140° C. for 5 h. After the completion of the reaction was confirmed by TLC, ice-cold water was added and extraction with EtOAc was performed. The combined organic layers were washed with sat. brine, dried over anhydrous sodium sulfate and evaporated under reduced pressure. Subsequent column chromatography of the obtained crude residue (SiO₂, 15-20% EtOAc in hexane) afforded tert-butyl N-[[1-(2-cyanophenyl)-4-piperidyl]methyl]carbamate as a colorless solid (1.30 g, 50%).

In a next step, the isolated compound (1.20 g, 3.80 mmol) was dissolved in anhydrous methylene chloride (30 mL) to which trifluoroacetic acid (0.58 mL, 7.61 mmol) was added at 0° C. Upon completion of the addition, the reaction was allowed to slowly warm up to room temperature and kept stirring at this temperature for 16 h. When complete conversion of the reaction was confirmed by TLC, a sat. NaHCO₃ solution was added to neutralize the reaction mixture and extraction with CH₂Cl₂ was performed. The combined organic layers were washed with sat. brine, dried over anhydrous Na₂SO₄ and concentrated in vacuo to deliver 2-[4-(aminomethyl)-1-piperidyl]benzonitrile as a pale-yellow liquid (573 mg, 70%).

To a stirred solution of 2-[4-(aminomethyl)-1-piperidyl]benzonitrile (500 mg, 2.32 mmol) in anhydrous EtOH (5 mL) was added 1H-3,1-benzoxazine-2,4-dione (455 mg, 2.79 mmol) and PTSA (40 mg, 0.23 mmol) after which the reaction was stirred at 100° C. for 5 h. At this point, trimethoxmethylbenzene (3.20 mL, 18.6 mmol) was added and stirring was continued at 100° C. for an additional 25 hours. Upon completion of the reaction (as determined by TLC), the reaction mixture was cooled down to 0° C. and the resulting precipitate was filtered off to yield 2-[4-[(4-oxo-2-phenyl-quinazolin-3-yl)methyl]-1-piperidyl]benzonitrile as a pale-yellow solid (528 mg, 54%).

A solution 2-[4-[(4-oxo-2-phenyl-quinazolin-3-yl)methyl]-1-piperidyl]benzonitrile (200 mg, 0.476 mmol), NaN₃ (247 mg, 3.80 mmol) and Bu₃SnCl (1.03 mL, 3.80 mmol) in toluene (5 mL) was stirred at 140° C. for 22 h in a sealed tube. After the completion of the reaction mixture was confirmed by TLC, the reaction mixture was evaporated under reduced pressure and the obtained residue was re-dissolved in methylene chloride. The organic layer was washed with a 10% NaOH solution and subsequently, the aqueous layer was neutralized with a citric acid solution. Extraction of the aq. phase with CH₂Cl₂ was performed. The combined organic layers were washed with sat. brine, dried over anhydrous sodium sulfate and concentrated in vacuo to afford a crude residue, which was further purified through column chromatography over silica gel (4-5% MeOH in CH₂Cl₂) to yield the envisaged tetrazole 2-phenyl-3-[[1-[2-(2H-tetrazol-5-yl)phenyl]-4-piperidyl]methyl]quinazolin-4-one A-249 as a yellow solid (42 mg, 19%).

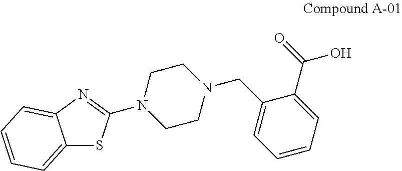 [SMILES: O=C(O)c1ccccc1CN1CCN(c2nc3ccccc3s2)CC1]

Prepared through method H by mixing 2-(piperazin-1-yl)benzo[d]thiazole (100 mg, 0.45 mmol) and 2-(bromomethyl)benzonitrile (89 mg, 0.45 mmol) for 2 h at r.t. Aqueous work-up with CH₂Cl₂, followed by chromatography on a column of silica gel using 70% EtOAc in hexane, yielded 2-[[4-(1,3-benzothiazol-2-yl)piperazin-1-yl]methyl]benzonitrile (20 mg, 13%) as a colorless solid in high purity.

Subsequent hydrolysis of the nitrile (450 mg, 1.34 mmol) in a 80% sulfuric acid solution was completed after overnight stirring at 90° C. Aq. work-up, followed by column chromatography (10% of MeOH in CH₂Cl₂), gave the envisaged compound A-01 as a colorless solid (290 mg, 61%).

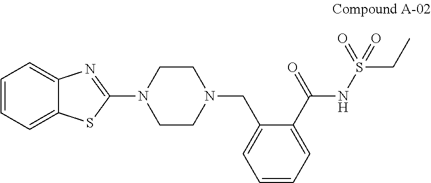 [SMILES: CCS(=O)(=O)NC(=O)c1ccccc1CN1CCN(c2nc3ccccc3s2)CC1]

Prepared through method H from a solution of 2-[[4-(1,3-benzothiazol-2-yl)piperazin-1-yl]methyl]benzoic acid (120 mg, 0.33 mmol), DCC (700 mg, 3.3 mmol), DMAP (41 mg, 0.33 mmol) and ethanesulfonamide (36 mg, 0.37 mmol) in anhydrous methylene chloride (5 mL). After stirring at room temperature for 24 h, an aq. work-up and column chromatography (silica gel using 75% EtOAc in hexane) was performed, yielding compound A-02 (18 mg, 11%) as a colorless solid in high purity.

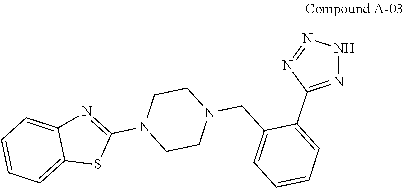 [SMILES: c1ccc(-c2nn[nH]n2)c(CN2CCN(c3nc4ccccc4s3)CC2)c1]

Prepared through method H from a solution of 2-[[4-(1,3-benzothiazol-2-yl)piperazin-1-yl]methyl]benzonitrile (50 mg, 0.15 mmol) and NaN₃ (100 mg, 1.5 mmol) in DMSO stirred at 120° C. for 16 h. An aq. work-up, followed by additional purification via a column of silica gel (10% methanol/90% methylene chloride), delivered compound A-03 (25 mg, 44%) as a colorless solid.

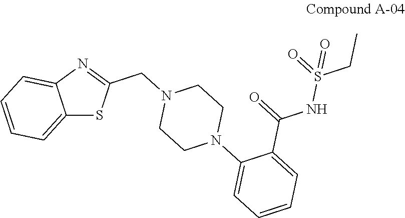 [SMILES: CCS(=O)(=O)NC(=O)c1ccccc1N1CCN(Cc2nc3ccccc3s2)CC1]

Prepared through method I.

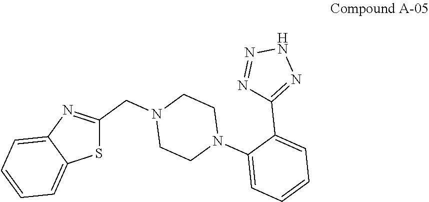 [SMILES: c1ccc(N2CCN(Cc3nc4ccccc4s3)CC2)c(-c2nn[nH]n2)c1]

Prepared through method I.

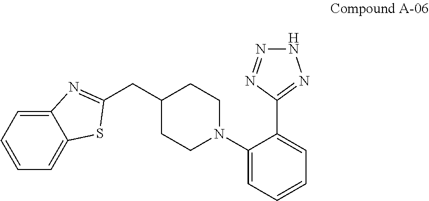 [SMILES: c1ccc(N2CCC(Cc3nc4ccccc4s3)CC2)c(-c2nn[nH]n2)c1]

Prepared through method J.

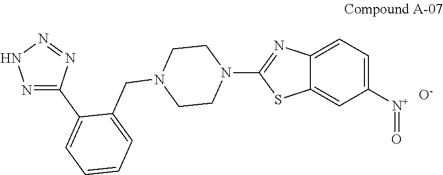

Prepared in a similar manner as described in method H by mixing 6-nitro-2-piperazin-1-yl)benzo[d]thiazole (200 mg, 0.757 mmol) and 2-(bromomethyl)benzonitrile (148 mg, 0.757 mmol) for 14 h at r.t. Aqueous work-up with CH₂Cl₂, followed by chromatography on a column of silica gel using 2-3% MeOH in CH₂Cl₂, yielded 2-[[4-(6-nitro-1,3-benzothiazol-2-yl)piperazin-1-yl]methyl]benzonitrile (138 mg, 48%) as a pale-brown solid in high purity. Subsequent tetrazole formation was performed, by mixing the substituted nitrile (70 mg, 0.184 mmol), NaN₃ (96 mg, 1.48 mmol) and Bu₃SnCl (0.40 mL, 1.48 mmol) in toluene (5 mL) at 140° C. for 18 h in a sealed tube. An aq. work-up, followed by additional purification via a column of silica gel (4-5% MeOH in CH₂Cl₂), delivered compound A-07 as a pale-yellow solid (40 mg, 51%).

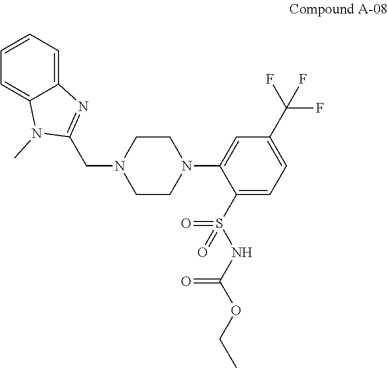 [SMILES: CCOC(=O)NS(=O)(=O)c1ccc(C(F)(F)F)cc1N1CCN(Cc2nc3ccccc3n2C)CC1]

Prepared through method K from 2-bromo-4-(trifluoromethyl)benzenesulfonamide (1.0 g, 3.289 mmol) to give 2-(piperazin-1-yl)-4-(trifluoromethyl)benzenesulfonamide as an off-white solid (0.90 g, 89%). By adding 2-(chloromethyl)-1-methyl-1H-benzo[d]imidazole and Et₃N to perform a nucleophilic substitution, the envisaged compound was obtained after 3 h at 100° C. After performing an aq. work-up and column chromatography (45-50% of ethyl acetate in hexane) 2-(4-((1-methyl-1H-benzo[d]imidazol-2-yl)methyl)piperazin-1-yl)-4-(trifluoromethyl)benzenesulfonamide was obtained as an off-white solid (0.62 g, 31%).

Mixing of the sulfonamide (0.15 g, 0.331 mmol) with ethyl (4-nitrophenyl) carbonate (104 mg, 0.496 mmol) and DBU (75 mg, 0.375 mmol) for 14 h at reflux temperature afforded the desired end product. Aq. work-up, followed by column chromatography (35-40% of ethyl acetate in hexane), yielded ethyl N-[2-[4-[(1-methylbenzimidazol-2-yl)methyl]piperazin-1-yl]-4-(trifluoromethyl)phenyl]sulfonylcarbamate as an off-white solid (35 mg, 20%).

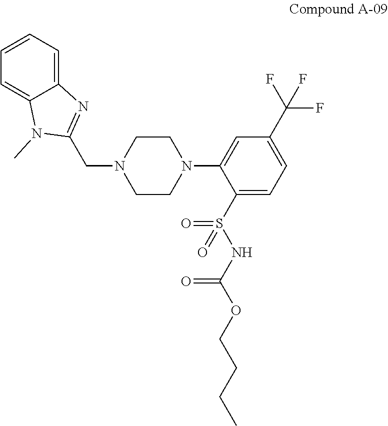 [SMILES: CCCCOC(=O)NS(=O)(=O)c1ccc(C(F)(F)F)cc1N1CCN(Cc2nc3ccccc3n2C)CC1]

Prepared through method K from 2-bromo-4-(trifluoromethyl)benzenesulfonamide (1.0 g, 3.289 mmol) to give 2-(piperazin-1-yl)-4-(trifluoromethyl)benzenesulfonamide as an off-white solid (0.90 g, 89%). By adding 2-(chloromethyl)-1-methyl-1H-benzo[d]imidazole and Et₃N to perform a nucleophilic substitution, the envisaged compound was obtained after 3 h at 100° C. After performing an aq. work-up and column chromatography (45-50% of ethyl acetate in hexane) 2-(4-((1-methyl-1H-benzo[d]imidazol-2-yl)methyl)piperazin-1-yl)-4-(trifluoromethyl)benzenesulfonamide was obtained as an off-white solid (0.62 g, 31%).

Mixing of the sulfonamide (0.075 g, 0.165 mmol) with butyl (4-nitrophenyl) carbonate (60 mg, 0.248 mmol) and DBU (57 mg, 0.375 mmol) for 14 h at reflux temperature afforded the desired end product. Aq. work-up, followed by column chromatography (35-40% of ethyl acetate in hexane), yielded butyl N-[2-[4-[(1-methylbenzimidazol-2-yl)methyl]piperazin-1-yl]-4-(trifluoromethyl)phenyl]sulfonylcarbamate as a white solid (10 mg, 14%).

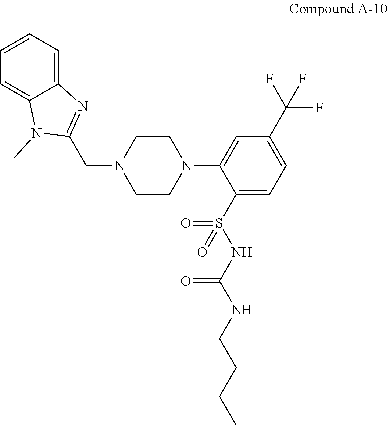 [SMILES: CCCCNC(=O)NS(=O)(=O)c1ccc(C(F)(F)F)cc1N1CCN(Cc2nc3ccccc3n2C)CC1]

Prepared through method K from 2-bromo-4-(trifluoromethyl)benzenesulfonamide (1.0 g, 3.289 mmol) to give 2-(piperazin-1-yl)-4-(trifluoromethyl)benzenesulfonamide as an off-white solid (0.90 g, 89%). By adding 2-(chloromethyl)-1-methyl-1H-benzo[d]imidazole and Et₃N to perform a nucleophilic substitution, the envisaged compound was obtained after 3 h at 100° C. After performing an aq. work-up and column chromatography (45-50% of ethyl acetate in hexane) 2-(4-((1-methyl-1H-benzo[d]imidazol-2-yl)methyl)piperazin-1-yl)-4-(trifluoromethyl)benzenesulfonamide was obtained as an off-white solid (0.62 g, 31%).

Mixing of the sulfonamide (0.15 g, 0.33 mmol) with 4-nitrophenyl butylcarbamate (0.118 g, 0.496 mmol), and DBU (75 mg, 0.496 mmol) for 14 h at reflux temperature afforded the desired end product. Aq. work-up, followed by column chromatography (35-40% of ethyl acetate in hexane), yielded 1-butyl-3-[2-[4-[(1-methylbenzimidazol-2-yl)methyl]piperazin-1-yl]-4-(trifluoromethyl)phenyl]sulfonyl-urea as an off-white solid (40 mg, 22%).

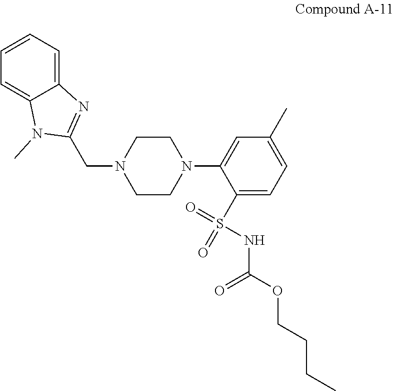 [SMILES: CCCCOC(=O)NS(=O)(=O)c1ccc(C)cc1N1CCN(Cc2nc3ccccc3n2C)CC1]

Prepared through method K from 2-fluoro-4-methyl-benzenesulfonamide (0.5 g, 2.65 mmol) to give 4-methyl-2-(piperazin-1-yl)benzenesulfonamide as an off-white solid (0.42 g, 63%). By adding 2-(chloromethyl)-1-methyl-1H-benzo[d]imidazole and Et₃N to perform a nucleophilic substitution, the envisaged compound was obtained after 3 h at 100° C. After performing an aq. work-up and column chromatography (45-50% of ethyl acetate in hexane) 4-methyl-2-(4-((1-methyl-1H-benzo[d]imidazol-2-yl)methyl)piperazin-1-yl)benzenesulfonamide was obtained as an off-white solid (0.46 g, 74%).

Mixing of the sulfonamide (0.1 g, 0.25 mmol) with butyl (4-nitrophenyl) carbonate (90 mg, 0.375 mmol) and DBU (57 mg, 0.375 mmol) for 14 h at reflux temperature afforded the desired end product. Aq. work-up, followed by column chromatography (35-40% of ethyl acetate in hexane), yielded butyl N-[4-methyl-2-[4-[(1-methylbenzimidazol-2-yl)methyl]piperazin-1-yl]phenyl]sulfonylcarbamate as a white solid (10 mg, 8%).

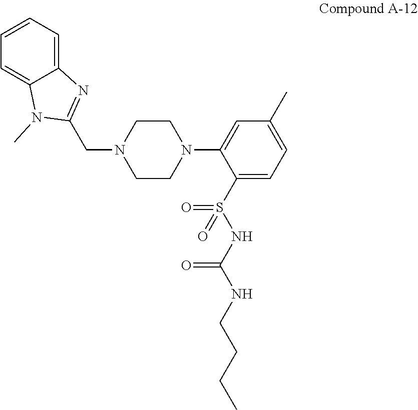 [SMILES: CCCCNC(=O)NS(=O)(=O)c1ccc(C)cc1N1CCN(Cc2nc3ccccc3n2C)CC1]

Prepared through method K from 2-fluoro-4-methyl-benzenesulfonamide (0.5 g, 2.65 mmol) to give 4-methyl-2-(piperazin-1-yl)benzenesulfonamide as an off-white solid (0.42 g, 63%). By adding 2-(chloromethyl)-1-methyl-1H-benzo[d]imidazole and Et₃N to perform a nucleophilic substitution, the envisaged compound was obtained after 3 h at 100° C. After performing an aq. work-up and column chromatography (45-50% of ethyl acetate in hexane) 4-methyl-2-(4-((1-methyl-1H-benzo[d]imidazol-2-yl)methyl)piperazin-1-yl)benzenesulfonamide was obtained as an off-white solid (0.46 g, 74%).

Mixing of the sulfonamide (0.1 g, 0.25 mmol) with 4-nitrophenyl butylcarbamate (90 mg, 0.375 mmol) and DBU (57 mg, 0.375 mmol) for 14 h at reflux temperature afforded the desired end product. Aq. work-up, followed by column chromatography (35-40% of ethyl acetate in hexane), yielded 1-butyl-3-[4-methyl-2-[4-[(1-methylbenzimidazol-2-yl)methyl]piperazin-1-yl]phenyl]sulfonyl-urea as a white solid (20 mg, 14%).

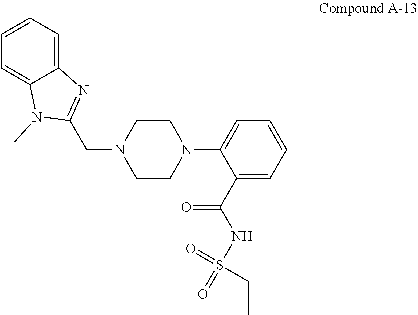 [SMILES: CCS(=O)(=O)NC(=O)c1ccccc1N1CCN(Cc2nc3ccccc3n2C)CC1]

Prepared through method N by adding 2-(chloromethyl)-1-methyl-1H-benzo[d]imidazole (0.32 mg, 1.74 mmol) to a solution of methyl 2-(piperazin-1-yl)benzoate (0.35 g, 1.58 mmol) and Et₃N (0.66 mL, 4.76 mmol) in DMF (20 mL) to perform a nucleophilic substitution. The envisaged compound was obtained after stirring the reaction mixture for 8 h at room temperature. After performing an aq. work-up and column chromatography (25-30% of ethyl acetate in hexane) methyl 2-(4-((1-methyl-1H-benzo[d]imidazol-2-yl)methyl)piperazin-1-yl)benzoate was obtained in moderate yield (195 mg, 34%).

Subsequent hydrolysis of the ester (190 mg, 0.52 mmol) in presence of lithium hydroxide monohydrate (25 mg, 1.04 mmol) in MeOH/H₂O (2:1) was completed after 3 h stirring at room temperature. Aq. work-up as described in method N delivered the crude acid (155 mg) which was used as such in the following reaction.

As such, mixing of the carboxylic acid (150 mg, 0.42 mmol) with CDI (139 mg, 0.85 mmol) in THE at 0° C., followed after 15 minutes by the addition of DBU (0.13 mL, 0.85 mmol) and ethanesulfonamide (46 mg, 0.47 mmol), afforded the envisaged end product after 12 h stirring of the reaction mixture at r.t. Aq. work-up, followed by column chromatography (4-5% MeOH in CH₂Cl₂), yielded N-ethylsulfonyl-2-[4-[(1-methylbenzimidazol-2-yl)methyl]piperazin-1-yl]benzamide as an off-white solid (34 mg, 18%).

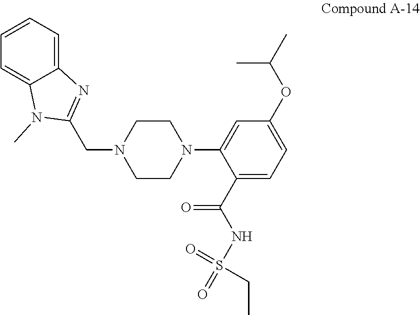 [SMILES: CCS(=O)(=O)NC(=O)c1ccc(OC(C)C)cc1N1CCN(Cc2nc3ccccc3n2C)CC1]

Prepared through method 0 by adding 2-(chloromethyl)-1-methyl-1H-benzo[d]imidazole (0.39 g, 2.15 mmol) to a solution of methyl 4-isopropoxy-2-(piperazin-1-yl)benzoate (0.5 g, 1.79 mmol) and Et₃N (0.62 mL, 4.49 mmol) in 1,4-dioxane (30 mL) to perform a nucleophilic substitution. The envisaged compound was obtained after stirring the reaction mixture for 3 h at 80° C. After performing an aq. work-up and column chromatography (30-40% of ethyl acetate in hexane) methyl 4-isopropoxy-2-(4-((1-methyl-1H-benzo[d]imidazol-2-yl)methyl)piperazin-1-yl)benzoate was obtained in moderate yield (380 mg, 50%).

Subsequent hydrolysis of the ester (370 mg, 0.88 mmol) in presence of lithium hydroxide monohydrate (42 mg, 1.75 mmol) in MeOH/H₂O (2:1) was completed after 4 h stirring at room temperature. Aq. work-up as described in method 0 delivered the crude acid which was used as such in the following reaction.

As such, mixing of the carboxylic acid (100 mg, 0.24 mmol) with CDI (79 mg, 0.49 mmol) in THE at 0° C., followed after 15 minutes by the addition of DBU (0.074 mL, 0.49 mmol) and ethanesulfonamide (32 mg, 0.29 mmol), afforded the envisaged end product after 11 h stirring of the reaction mixture at r.t. Aq. work-up, followed by column chromatography (5-6% MeOH in CH₂Cl₂), yielded N-ethylsulfonyl-4-isopropoxy-2-[4-[(1-methylbenzimidazol-2-yl)methyl]piperazin-1-yl]benzamide as a pale-yellow solid (10 mg, 8%).

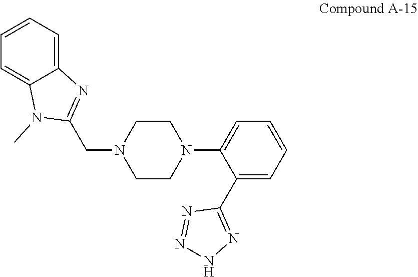 [SMILES: Cn1c(CN2CCN(c3ccccc3-c3nn[nH]n3)CC2)nc2ccccc21]

Prepared through method L from 2-(piperazin-1-yl)benzonitrile (334 mg, 1.78 mmol) in DMF (40 mL), to which Et₃N (0.74 mL, 5.34 mmol) and 2-(chloromethyl)-1-methyl-1H-benzo[d]imidazole (354 mg, 1.96 mmol) were added. The reaction mixture was stirred at r.t. for 6 h to afford the envisaged nucleophilic substitution compound as an off-white solid (265 mg, 45%).

Final tetrazole reaction was performed, by mixing the nitrile (260 mg, 0.78 mmol), NaN₃ (408 mg, 6.28 mmol) and Bu₃SnCl (1.54 mL, 6.28 mmol) in xylene (8 mL) at 140° C. for 18 h in a sealed tube. Aq. work-up, followed by trituration with n-pentane, afforded the desired tetrazole as an off-white solid (8 mg, 10%).

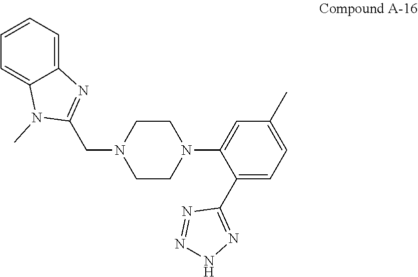 [SMILES: Cc1ccc(-c2nn[nH]n2)c(N2CCN(Cc3nc4ccccc4n3C)CC2)c1]

Prepared through method L from 4-methyl-2-(piperazin-1-yl)benzonitrile (400 mg, 1.98 mmol) in 1,4-dioxane (40 mL), to which Et₃N (0.82 mL, 6.0 mmol) and 2-(chloromethyl)-1-methyl-1H-benzo[d]imidazole (400 mg, 2.2 mmol) were added. The reaction mixture was stirred at 100° C. for 6 h to afford the envisaged nucleophilic substitution compound as an off-white solid (306 mg, 45%).

Final tetrazole reaction was performed, by mixing the nitrile (300 mg, 0.87 mmol), NaN₃ (451 mg, 6.95 mmol) and Bu₃SnCl (1.7 mL, 6.95 mmol) in toluene (8 mL) at 150° C. for 22 h in a sealed tube. Aq. work-up, followed by trituration with n-pentane, afforded the desired tetrazole as a pale-yellow solid with enriched purity (13 mg, 4%).

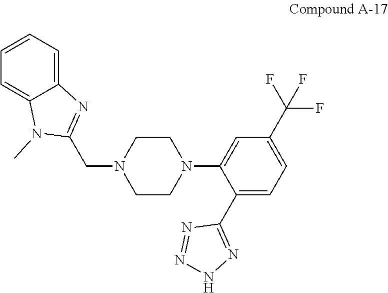 [SMILES: Cn1c(CN2CCN(c3cc(C(F)(F)F)ccc3-c3nn[nH]n3)CC2)nc2ccccc21]

Prepared through method L from 2-(piperazin-1-yl)-4-(trifluoromethyl)benzonitrile (500 mg, 1.95 mmol) in 1,4-dioxane (50 mL), to which Et₃N (0.81 mL, 5.87 mmol) and 2-(chloromethyl)-1-methyl-1H-benzo[d]imidazole (353 mg, 2.2 mmol) were added. The reaction mixture was stirred at 100° C. for 5 h to afford the envisaged nitrile as an off-white solid (352 mg, 45%).

Final tetrazole reaction was performed, by mixing the nitrile (300 mg, 0.75 mmol), NaN₃ (390 mg, 6.00 mmol) and Bu₃SnCl (1.5 mL, 6.00 mmol) in toluene (8 mL) at 150° C. for 24 h in a sealed tube. Aq. work-up, followed by trituration with n-pentane, afforded the desired tetrazole as a colorless solid with enriched purity (19 mg, 6%).

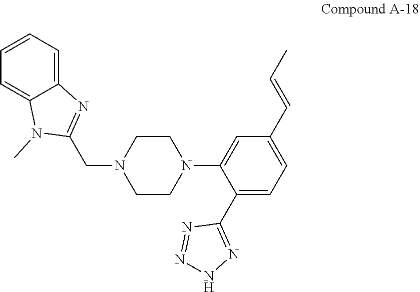 [SMILES: C/C=C/c1ccc(-c2nn[nH]n2)c(N2CCN(Cc3nc4ccccc4n3C)CC2)c1]

Prepared through method L from intermediate 11 (270 mg, 1.18 mmol) in 1,4-dioxane (25 mL), to which Et₃N (0.5 mL, 3.56 mmol) and 2-(chloromethyl)-1-methyl-1H-benzo[d]imidazole (236 mg, 1.3 mmol) were added. The reaction mixture was stirred at room temperature for 4 h to afford the envisaged nucleophilic substitution compound after aq. work-up and column chromatography (SiO₂, 30-40% EtOAc in hexane) as a pale-yellow solid (284 mg, 64%).

Final tetrazole reaction was performed, by mixing the nitrile (140 mg, 0.37 mmol), NaN₃ (196 mg, 3.0 mmol) and Bu₃SnCl (0.75 mL, 3.0 mmol) in toluene (5 mL) at 150° C. for 20 h in a sealed tube. Aq. work-up, followed by column chromatography (SiO₂, 3-4% MeOH in CH₂Cl₂), afforded the desired tetrazole as a pale-yellow solid (18 mg, 12%).

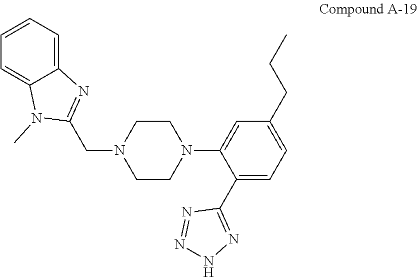 [SMILES: CCCc1ccc(-c2nn[nH]n2)c(N2CCN(Cc3nc4ccccc4n3C)CC2)c1]

A stirred solution of 2-(4-((1-methyl-1H-benzo[d]imidazol-2-yl)methyl)piperazin-1-yl)-4-(prop-1-en-1-yl)benzonitrile (140 mg, 0.37 mmol) in MeOH (20 mL) was hydrogenated over 10% Pd/C (15.0 mg) under 5 Kg/cm² H₂ pressure using a Parr hydrogenator for 3 h at ambient temperature. After confirming the completion of reaction by LC-MS, the reaction mixture was filtered through a Celite® bed and was evaporated in vacuo. The colorless gummy liquid (93 mg, 65%) was taken to the next step without further purification.

Final tetrazole reaction was performed (as described in the last step of method L), by mixing the nitrile (90 mg, 0.24 mmol), NaN₃ (125 mg, 1.9 mmol) and Bu₃SnCl (0.48 mL, 1.9 mmol) in toluene (5 mL) at 150° C. for 22 h in a sealed tube. Aq. work-up, followed by column chromatography (SiO₂, 4-5% MeOH in CH₂Cl₂), afforded the desired tetrazole as an off-white solid (13 mg, 13%).

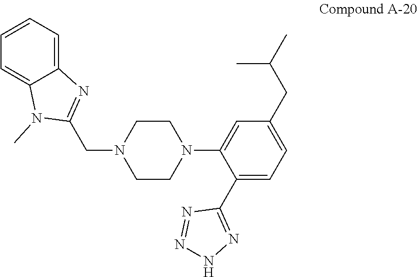 [SMILES: CC(C)Cc1ccc(-c2nn[nH]n2)c(N2CCN(Cc3nc4ccccc4n3C)CC2)c1]

A stirred solution of 2-(4-((1-methyl-1H-benzo[d]imidazol-2-yl)methyl)piperazin-1-yl)-4-(2-methylprop-1-en-1-yl)benzonitrile (85 mg, 0.22 mmol) in MeOH (10 mL) was hydrogenated over 10% Pd/C (15.0 mg) under 5 Kg/cm² H₂ pressure using a Parr hydrogenator for 3 h at ambient temperature. After confirming the completion of reaction by LC-MS, the reaction mixture was filtered through a Celite® bed and was evaporated in vacuo. The colorless gummy liquid (60 mg, 70%) was taken to the next step without further purification.

Final tetrazole reaction was performed (as described in the last step of method L), by mixing the nitrile (60 mg, 0.155 mmol), NaN₃ (81 mg, 1.24 mmol) and Bu₃SnCl (0.34 mL, 1.24 mmol) in toluene (5 mL) at 150° C. for 20 h in a sealed tube. Aq. work-up, followed by column chromatography (SiO₂, 4-5% MeOH in CH₂Cl₂), yielded the envisaged tetrazole as a colorless solid (8 mg, 12%).

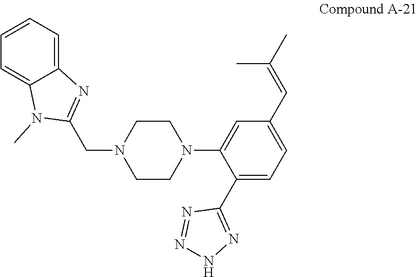 [SMILES: CC(C)=Cc1ccc(-c2nn[nH]n2)c(N2CCN(Cc3nc4ccccc4n3C)CC2)c1]

To a stirred solution of 4-bromo-2-(piperazin-1-yl)benzonitrile (200 mg, 0.75 mmol) in 1,4-dioxane (20 mL), was added Et₃N (0.31 mL, 2.25 mmol) at 0° C. after which the reaction was allowed to warm up to room temperature. After 10 minutes, 2-(chloromethyl)-1-methyl-1H-benzo[d]imidazole (149 mg, 0.82 mmol) was added and the resulting mixture was kept stirring at r.t. for 4 h. After completion of the reaction was confirmed by TLC, the reaction mixture was diluted with water and extracted with ethyl acetate. The combined organic layers were washed with water, sat. brine, dried over anhydrous sodium sulfate and evaporated under reduced pressure to afford a crude residue. Column chromatography over silica gel (30-40% EtOAc in hexane), yielded the desired nitrile (153 mg, 49%).

To a stirred solution of 4-bromo-2-(4-((1-methyl-1H-benzo[d]imidazol-2-yl)methyl)piperazin-1-yl)benzonitrile (150 mg, 0.37 mmol) in 1,4-dioxane (20 mL) was added 4,4,5,5-tetramethyl-2-(2-methylprop-1-en-1-yl)-1,3,2-dioxaborolane (73 mg, 0.4 mmol), followed by K₂CO₃ (151 mg, 1.0 mmol) and the resultant mixture was bubbled with argon for 20 min. Then Pd(dppf)Cl₂ (27 mg, 0.04 mmol) was added after which the reaction was heated to 80° C. for 6 h. After the completion of the reaction was confirmed by TLC, the reaction mixture was evaporated in vacuo to remove the volatiles and the residue was re-dissolved with ethyl acetate and washed with water and sat. brine. Subsequent drying over anhydrous sodium sulfate, solvent evaporation in vacuo and column chromatography (SiO₂, 4-5% MeOH in CH₂Cl₂), afforded 2-(4-((1-methyl-1H-benzo[d]imidazol-2-yl)methyl)piperazin-1-yl)-4-(2-methylprop-i-en-1-yl)benzonitrile (89 mg, 63%) as an off-white solid.

Final tetrazole reaction as described in the last step of method L was performed, by mixing the nitrile (80 mg, 0.20 mmol), NaN₃ (108 mg, 1.7 mmol) and Bu₃SnCl (0.42 mL, 1.7 mmol) in toluene (5 mL) at 150° C. for 20 h in a sealed tube. Aq. work-up, followed by column chromatography (SiO₂, 4-5% MeOH in CH₂Cl₂), afforded the envisaged tetrazole as a colorless solid (7 mg, 8%).

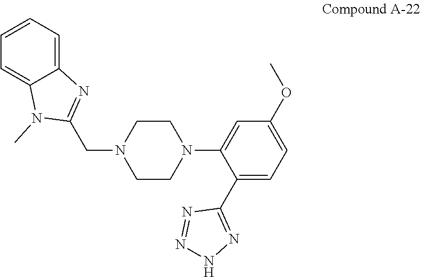 [SMILES: COc1ccc(-c2nn[nH]n2)c(N2CCN(Cc3nc4ccccc4n3C)CC2)c1]

Prepared through method L from 4-methoxy-2-(piperazin-1-yl)benzonitrile (500 mg, 2.30 mmol) in 1,4-dioxane (30 mL), to which Et₃N (0.82 mL, 6.9 mmol) and 2-(chloromethyl)-1-methyl-1H-benzo[d]imidazole (400 mg, 2.5 mmol) were added. The reaction mixture was stirred at 100° C. for 6 h to afford 4-methoxy-2-(4-((1-methyl-1H-benzo[d]imidazol-2-yl)methyl)piperazin-1-yl)benzonitrile as pale-yellow solid (358 mg, 43%).

Final tetrazole reaction was performed, by mixing the nitrile (350 mg, 0.9 mmol), NaN₃ (503 mg, 7.7 mmol) and Bu₃SnCl (1.66 mL, 7.7 mmol) in toluene (8 mL) at 150° C. for 20 h in a sealed tube. Aq. work-up, followed by column chromatography over silica gel (5% MeOH in CH₂Cl₂), delivered 2-[[4-[5-methoxy-2-(2H-tetrazol-5-yl)phenyl]piperazin-1-yl]methyl]-1-methyl-benzimidazole as a pale-yellow solid (12 mg, 3%).

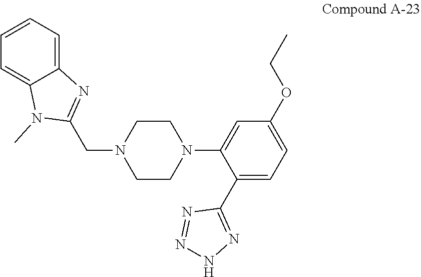 [SMILES: CCOc1ccc(-c2nn[nH]n2)c(N2CCN(Cc3nc4ccccc4n3C)CC2)c1]

Prepared through method L from 4-ethoxy-2-(piperazin-1-yl)benzonitrile (500 mg, 2.16 mmol) in 1,4-dioxane (30 mL), to which Et₃N (0.89 mL, 6.5 mmol) and 2-(chloromethyl)-1-methyl-1H-benzo[d]imidazole (430 mg, 2.4 mmol) were added. The reaction mixture was stirred at 100° C. for 5 h to afford the desired nitrile as an off-white solid (304 mg, 37%).

Final tetrazole reaction was performed, by mixing the nitrile (300 mg, 0.8 mmol), NaN₃ (415 mg, 6.4 mmol) and Bu₃SnCl (1.60 mL, 6.4 mmol) in toluene (8 mL) at 150° C. for 22 h in a sealed tube. Aq. work-up, followed by column chromatography (SiO₂, 5% MeOH in CH₂Cl₂), afforded the desired tetrazole as an off-white solid (12 mg, 4%).

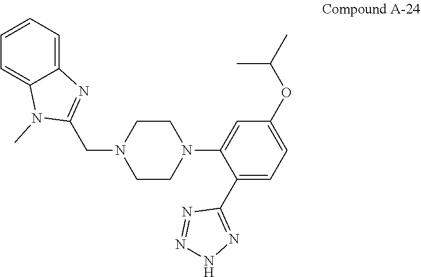 [SMILES: CC(C)Oc1ccc(-c2nn[nH]n2)c(N2CCN(Cc3nc4ccccc4n3C)CC2)c1]

Prepared through method L from 4-isopropoxy-2-(piperazin-1-yl)benzonitrile (500 mg, 2.04 mmol) in 1,4-dioxane (30 mL), to which Et₃N (0.85 mL, 6.11 mmol) and 2-(chloromethyl)-1-methyl-1H-benzo[d]imidazole (405 mg, 2.24 mmol) were added. The reaction mixture was stirred at 100° C. for 5 h to afford the desired nitrile as an off-white solid (365 mg, 46%).

Final tetrazole reaction was performed, by mixing the nitrile (250 mg, 0.6 mmol), NaN₃ (333 mg, 5.1 mmol), and Bu₃SnCl (1.28 mL, 5.1 mmol) in toluene (8 mL) at 150° C. for 21 h in a sealed tube. Aq. work-up, followed by column chromatography (SiO₂, 5% MeOH in CH₂Cl₂), afforded 2-[[4-[5-isopropoxy-2-(2H-tetrazol-5-yl)phenyl]piperazin-1-yl]methyl]-1-methyl-benzimidazole as a pale-yellow solid (8 mg, 3%).

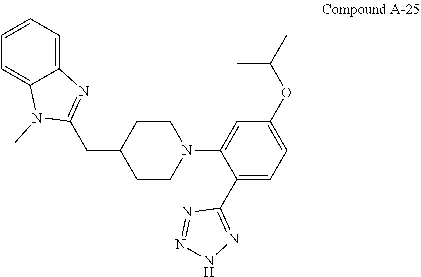 [SMILES: CC(C)Oc1ccc(-c2nn[nH]n2)c(N2CCC(Cc3nc4ccccc4n3C)CC2)c1]

Prepared through method P.

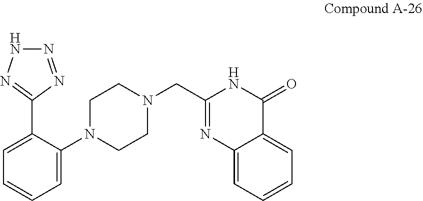 [SMILES: O=c1[nH]c(CN2CCN(c3ccccc3-c3nn[nH]n3)CC2)nc2ccccc12]

Prepared through method M from 2-(piperazin-1-yl)benzonitrile (70 mg, 0.38 mmol) in DMF (8 mL), to which Et₃N (0.16 mL, 1.13 mmol) and 2-(chloromethyl)quinazolin-4-(3H)-one (80 mg, 0.41 mmol) were added. The reaction mixture was stirred at r.t. for 8 h to afford the envisaged nucleophilic substitution compound as a pale-yellow solid (70 mg, 54%).

Final tetrazole reaction was performed, by mixing the nitrile (69 mg, 0.20 mmol), NaN₃ (104 mg, 1.60 mmol) and Bu₃SnCl (0.39 mL, 1.60 mmol) in xylene (5 mL) at 140° C. for 22 h in a sealed tube. Aq. work-up, followed by trituration with n-pentane, afforded the desired tetrazole as a brown colored solid (38 mg, 50%).

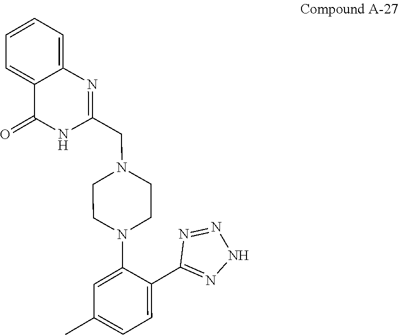 [SMILES: Cc1ccc(-c2nn[nH]n2)c(N2CCN(Cc3nc4ccccc4c(=O)[nH]3)CC2)c1]

Prepared through method M from 4-methyl-2-(piperazin-1-yl)benzonitrile (400 mg, 1.99 mmol) in DMF (30 mL), to which Et₃N (0.83 mL, 5.96 mmol) and 2-(chloromethyl)quinazolin-4-(3H)-one (425 mg, 2.19 mmol) were added. The reaction mixture was stirred at 100° C. for 8 h to afford the envisaged nucleophilic substitution compound as an off-white solid (321 mg, 45%).

Final tetrazole reaction was performed, by mixing the nitrile (300 mg, 0.83 mmol), NaN₃ (434 mg, 6.68 mmol) and Bu₃SnCl (1.7 mL, 6.68 mmol) in toluene (15 mL) at 150° C. for 22 h in a sealed tube. Aq. work-up, followed by trituration with n-pentane, afforded the desired tetrazole as an off-white solid with enriched purity (26 mg, 8%).

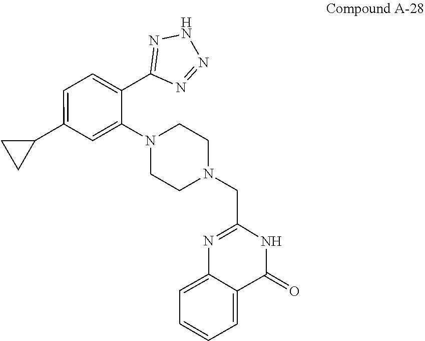 [SMILES: O=c1[nH]c(CN2CCN(c3cc(C4CC4)ccc3-c3nn[nH]n3)CC2)nc2ccccc12]

Prepared through method M from intermediate 37 (0.100 g, 0.440 mmol) and 2-(chloromethyl)quinazolin-4-(3H)-one (0.128 g, 0.660 mmol). The resulting reaction mixture was stirred at 80° C. for 6 h. Aq. work-up with EtOAc and column chromatography (45-50% EtOAc in hexane) yielded 4-4-cyclopropyl-2-(4-((4-oxo-3,4-dihydroquinazolin-2-yl)methyl)piperazin-1-yl)benzonitrile in good yield (0.11 g, 65%).

Final tetrazole reaction was performed, by mixing the nitrile (0.10 g, 0.259 mmol), sodium azide (0.135 g, 2.076 mmol) and Bu₃SnCl (0.676 g, 2.076 mmol) in toluene (10 mL) at 140° C. for 15 h in a sealed tube. Aq. work-up, as described in method M, followed by column chromatography (SiO₂, 5-8% MeOH in CH₂Cl₂) and final trituration using diethyl ether, afforded 2-[[4-[5-cyclopropyl-2-(2H-tetrazol-5-yl)phenyl]piperazin-1-yl]methyl]-3H-quinazolin-4-one as an off-white solid (11 mg, 10%)

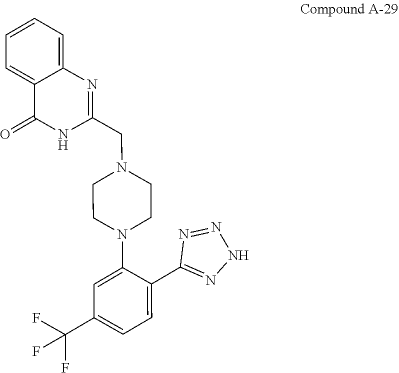 [SMILES: O=c1[nH]c(CN2CCN(c3cc(C(F)(F)F)ccc3-c3nn[nH]n3)CC2)nc2ccccc12]

Prepared through method M from 2-(piperazin-1-yl)-4-(trifluoromethyl)benzonitrile (400 mg, 1.57 mmol) in DMF (30 mL), to which Et₃N (0.66 mL, 4.70 mmol) and 2-(chloromethyl)quinazolin-4-(3H)-one (335 mg, 1.72 mmol) were added. The reaction mixture was stirred at 100° C. for 8 h to afford the envisaged nucleophilic substitution compound as an off-white solid (343 mg, 53%).

Final tetrazole reaction was performed, by mixing the nitrile (300 mg, 0.73 mmol), NaN₃ (377 mg, 5.80 mmol) and Bu₃SnCl (1.45 mL, 5.80 mmol) in toluene (15 mL) at 150° C. for 24 h in a sealed tube. Aq. work-up, followed by trituration with n-pentane, afforded the envisaged tetrazole as an off-white solid with enriched purity (31 mg, 9%).

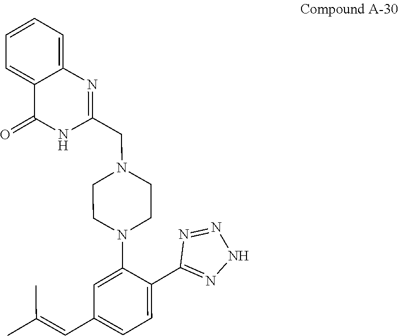 [SMILES: CC(C)=Cc1ccc(-c2nn[nH]n2)c(N2CCN(Cc3nc4ccccc4c(=O)[nH]3)CC2)c1]

Prepared through method M from 4-(2-methylprop-1-en-1-yl)-2-(piperazin-1-yl)benzonitrile (Scheme for intermediate 1, obtained after first 2 steps) (500 mg crude, 2.07 mmol) in DMF (40 mL), to which Et₃N (0.86 mL, 6.21 mmol) and 2-(chloromethyl)quinazolin-4-(3H)-one (484 mg, 2.5 mmol) were added. The resulting reaction mixture was stirred at r.t. for 8 h. Aq. work-up and column chromatography (2-3% MeOH in CH₂Cl₂) afforded 4-(2-methylprop-1-en-1-yl)-2-(4-((4-oxo-3,4-dihydroquinazolin-2-yl)methyl)piperazin-1-yl)benzonitrile in moderate yield (403 mg, 49%).

Final tetrazole reaction was performed, by mixing the nitrile (80 mg, 0.20 mmol), NaN₃ (104 mg, 1.6 mmol) and Bu₃SnCl (0.40 mL, 1.6 mmol) in toluene (5 mL) at 150° C. for 22 h in a sealed tube. Aq. work-up, followed by column chromatography (SiO₂, 4-5% MeOH in CH₂Cl₂), afforded the envisaged tetrazole as an off-white solid (23 mg, 26%).

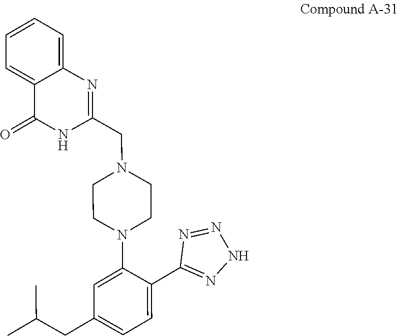 [SMILES: CC(C)Cc1ccc(-c2nn[nH]n2)c(N2CCN(Cc3nc4ccccc4c(=O)[nH]3)CC2)c1]

A stirred solution of 4-(2-methylprop-1-en-1-yl)-2-(4-((4-oxo-3,4-dihydroquinazolin-2-yl)methyl)piperazin-1-yl)benzonitrile (300 mg, 0.37 mmol) in MeOH (20 mL) was hydrogenated over 10% Pd/C (30 mg) under 5 Kg/cm² H₂ pressure using a Parr hydrogenator for 3 h at ambient temperature. After confirming the completion of the reaction by LC-MS, the reaction mixture was filtered through a Celite® bed and was evaporated in vacuo. The colorless gummy solid (240 mg) was taken to the next step without additional purification.

Final tetrazole reaction was performed (as described in the last step of method M), by mixing the nitrile (230 mg, 0.57 mmol), NaN₃ (298 mg, 4.6 mmol) and Bu₃SnCl (1.15 mL, 4.6 mmol) in toluene (15 mL) at 150° C. for 24 h in a sealed tube. Aq. work-up, followed by column chromatography (SiO₂, 4-5% MeOH in CH₂Cl₂), afforded 2-[[4-[5-isobutyl-2-(2H-tetrazol-5-yl)phenyl]piperazin-1-yl]methyl]-3H-quinazolin-4-one as a pale-yellow solid (34 mg, 13%).

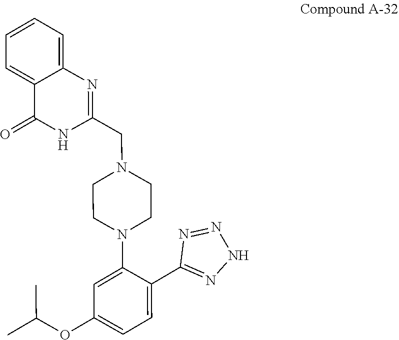 [SMILES: CC(C)Oc1ccc(-c2nn[nH]n2)c(N2CCN(Cc3nc4ccccc4c(=O)[nH]3)CC2)c1]

Prepared through method M from 4-isopropoxy-2-(piperazin-1-yl)benzonitrile (500 mg, 2.04 mmol) in DMF (30 mL), to which Et₃N (0.85 mL, 6.11 mmol) and 2-(chloromethyl)quinazolin-4-(3H)-one (436 mg, 2.24 mmol) were added. The reaction mixture was stirred at 100° C. for 10 h to afford the desired nitrile as an off-white solid (321 mg, 39%).

Final tetrazole reaction was performed, by mixing the nitrile (300 mg, 0.74 mmol), NaN₃ (389 mg, 5.94 mmol) and Bu₃SnCl (1.49 mL, 5.94 mmol) in toluene (15 mL) at 150° C. for 24 h in a sealed tube. Aq. work-up, followed by trituration with n-pentane, afforded the desired tetrazole as an off-white solid (13 mg, 4%).

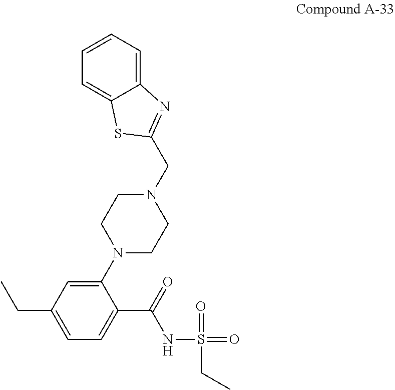 [SMILES: CCc1ccc(C(=O)NS(=O)(=O)CC)c(N2CCN(Cc3nc4ccccc4s3)CC2)c1]

Prepared through method B. To a stirred solution of intermediate 3 (160 mg, 0.36 mmol) in 1,4-dioxane (15 mL) was added ethylboronic acid (32 mg, 0.43 mmol), followed by K₂CO₃ (124 mg, 0.90 mmol) and the resultant mixture was bubbled with argon for 20 min. Then Pd(dppf)Cl₂ (26 mg, 0.04 mmol) was added after which the reaction was heated to 80° C. for 10 h. Upon completion of the reaction, the reaction mixture was evaporated in vacuo to remove the volatiles and the residue was re-dissolved with ethyl acetate and washed with water and sat. brine. Drying over anhydrous sodium sulfate and concentration under reduced pressure, followed by column chromatography over silica gel (40-45% EtOAc in hexane), yielded the envisaged compound (124 mg, 88%).

Subsequent hydrolysis of the ester (120 mg, 0.30 mmol) in presence of lithium hydroxide monohydrate (15 mg, 0.61 mmol) in MeOH:H₂O (10 mL, 2:1 ratio) was completed after 4 h stirring at room temperature. Aq. work-up as described in method B delivered the crude acid (108 mg crude) which was used as such in the following reaction.

As such, mixing of the carboxylic acid (100 mg, 0.26 mmol) with CDI (85 mg, 0.52 mmol) in THE (10 mL) at 0° C., followed after 15 minutes by the addition of DBU (0.08 mL, 0.52 mmol) and ethanesulfonamide (34 mg, 0.31 mmol), afforded the envisaged end product after 10 h stirring of the reaction mixture at r.t. Aq. work-up, followed by column chromatography (6-7% MeOH in CH₂Cl₂), yielded 2-[4-(1,3-benzothiazol-2-ylmethyl)piperazin-1-yl]-4-ethyl-N-ethylsulfonyl-benzamide as an off-white solid (10 mg, 8%).

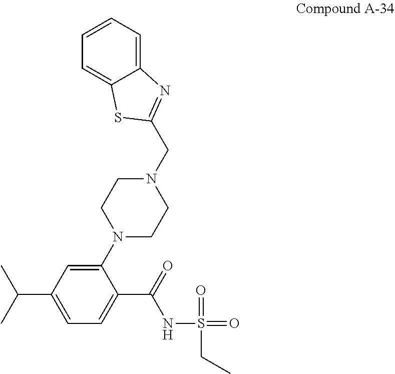 [SMILES: CCS(=O)(=O)NC(=O)c1ccc(C(C)C)cc1N1CCN(Cc2nc3ccccc3s2)CC1]

Prepared through method B. To a stirred solution of intermediate 3 (0.5 g, 1.12 mmol) in 1,4-dioxane (50 mL) was added 4,4,5,5-tetramethyl-2-(prop-1-en-2-yl)-1,3,2-dioxaborolane (0.23 g, 1.34 mmol), followed by K₂CO₃ (0.39 g, 2.80 mmol) and the resultant mixture was bubbled with argon for 20 min. Then Pd(dppf)Cl₂ (0.08 g, 0.11 mmol) was added after which the reaction was heated to 80° C. for 12 h. The reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (40-45% EtOAc in hexane) yielded the desired compound (384 mg, 84%).

A solution of methyl 2-(4-(benzo[d]thiazol-2-ylmethyl)piperazin-1-yl)-4-(prop-1-en-2-yl)benzoate (380 mg, 0.93 mmol) in MeOH (25 mL) was hydrogenated over 10% Pd/C (40 mg) under 5 Kg/cm² H₂ pressure using a Parr hydrogenator for 5 h at ambient temperature. The reaction mixture was filtered over a Celite Bed® and concentrated under reduced pressure. The crude compound was further triturated with diethyl ether to obtain methyl 2-(4-(benzo[d]thiazol-2-ylmethyl)piperazin-1-yl)-4-isopropylbenzoate as colorless gummy solid (307 mg).

A solution of methyl 2-(4-(benzo[d]thiazol-2-ylmethyl)piperazin-1-yl)-4-isopropylbenzoate (300 mg, 0.73 mmol) and lithium hydroxide monohydrate (35 mg, 1.46 mmol) in MeOH:H₂O (25 mL, 2:1 ratio) was stirred at room temperature for 5 h. After completion of the hydrolysis reaction, the mixture was concentrated under reduced pressure to get rid of the volatiles. Water was added to the organic residue and back washed with ethyl acetate. The aqueous phase was subsequently neutralized with 1M citric acid and extraction with ethyl acetate was performed. The combined organic layers were dried over anhydrous Na₂SO₄, filtered and concentrated under reduced pressure to afford the crude carboxylic acid (178 mg) which was taken for next step without purification.

To a solution of the carboxylic acid (170 mg, 0.43 mmol) in THE (20 mL) was added CDI (139 mg, 0.86 mmol) at 0° C., after which the reaction was left stirring at room temperature for 15 minutes. Next, DBU (0.13 mL, 0.86 mmol) and ethanesulfonamide (56 mg, 0.52 mmol) were added, after which the reaction mixture was kept at room temperature for 12 h. After confirmation of the completion of the reaction by TLC, the solution was evaporated to dryness, water was added followed by extraction with ethyl acetate. The combined organic layers were washed with sat. brine, dried over anhydrous sodium sulfate and evaporated in vacuo. Finally, column chromatography over silica gel (7-8% MeOH in CH₂Cl₂) allowed isolation of the envisaged compound as an off-white solid (40 mg, 19%).

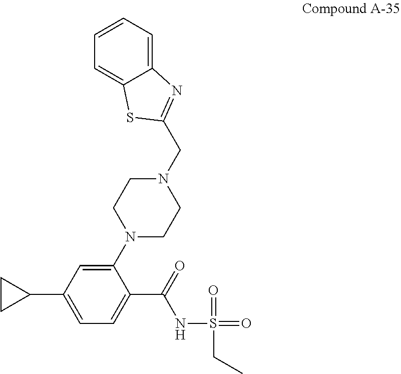 [SMILES: CCS(=O)(=O)NC(=O)c1ccc(C2CC2)cc1N1CCN(Cc2nc3ccccc3s2)CC1]

Prepared through method B. To a stirred solution of intermediate 3 (400 mg, 0.90 mmol) in 1,4-dioxane (50 mL) was added cyclopropylboronic acid (230 mg, 2.69 mmol), followed by K₂CO₃ (310 mg, 2.24 mmol) and the resultant mixture was bubbled with argon for 20 min. Then Pd(dppf)Cl₂ (66 mg, 0.09 mmol) was added after which the reaction was heated to 80° C. for 12 h. Upon completion of the reaction, the reaction mixture was evaporated in vacuo to remove the volatiles and the residue was re-dissolved with ethyl acetate and washed with water and sat. brine. Drying over anhydrous sodium sulfate and concentration under reduced pressure, followed by column chromatography over silica gel (45-50% EtOAc in hexane) yielded the envisaged compound (309 mg, 85%).

Subsequent hydrolysis of the ester (300 mg, 0.74 mmol) in presence of lithium hydroxide monohydrate (35 mg, 1.47 mmol) in MeOH:H₂O (15 mL, 2:1 ratio) was completed after 5 h stirring at room temperature. Aq. work-up as described in method B delivered the crude acid (158 mg crude) which was used as such in the following reaction.

As such, mixing of the carboxylic acid (150 mg, 0.38 mmol) with CDI (124 mg, 0.76 mmol) in THE (20 mL) at 0° C., followed after 15 minutes by the addition of DBU (0.11 mL, 0.76 mmol) and ethanesulfonamide (50 mg, 0.46 mmol), afforded the envisaged end product after 8 h stirring of the reaction mixture at r.t. Aq. work-up, followed by column chromatography (6-7% MeOH in CH₂Cl₂), yielded 2-[4-(1,3-benzothiazol-2-ylmethyl)piperazin-1-yl]-4-cyclopropyl-N-ethylsulfonyl-benzamide as an off-white solid (30 mg, 16%).

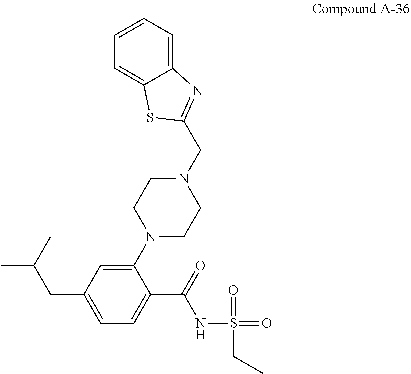 [SMILES: CCS(=O)(=O)NC(=O)c1ccc(CC(C)C)cc1N1CCN(Cc2nc3ccccc3s2)CC1]

Prepared through method B. To a stirred solution of intermediate 3 (250 mg, 0.56 mmol) in 1,4-dioxane (25 mL) was added 4,4,5,5-tetramethyl-2-(2-methylprop-1-en-1-yl)-1,3,2-dioxaborolane (122 mg, 0.67 mmol), followed by K₂CO₃ (193 mg, 1.40 mmol) and the resultant mixture was bubbled with argon for 20 min. Then Pd(dppf)Cl₂ (40 mg, 0.06 mmol) was added after which the reaction was heated to 80° C. for 10 h. When complete conversion was confirmed by TLC, the reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (35-40% EtOAc in hexane), yielded the compound of interest (214 mg, 91%).

A solution of methyl 2-(4-(benzo[d]thiazol-2-ylmethyl)piperazin-1-yl)-4-(2-methylprop-1-en-1-yl)benzoate (210 mg, 0.50 mmol) in MeOH (20 mL) was hydrogenated over 10% Pd/C (20 mg) under 5 Kg/cm² H₂ pressure using a Parr hydrogenator for 6 h at ambient temperature. The reaction mixture was filtered over a Celite Bed® and concentrated under reduced pressure. The crude compound was further triturated with diethyl ether to obtain methyl 2-(4-(benzo[d]thiazol-2-ylmethyl)piperazin-1-yl)-4-isobutylbenzoate as colorless gummy solid (183 mg).

Subsequent hydrolysis of the ester (180 mg, 0.43 mmol) in presence of lithium hydroxide monohydrate (20 mg, 0.86 mmol) in MeOH:H₂O (24 mL, 2:1 ratio) was completed after 5 h stirring at room temperature. Aq. work-up as described in method B delivered the crude acid (126 mg crude) which was used as such in the following reaction without further purification.

To a solution of the carboxylic acid (120 mg, 0.29 mmol) in THE (15 mL) was added CDI (95 mg, 0.59 mmol) at 0° C., after which the reaction was left stirring at room temperature for 15 minutes. Next, DBU (0.09 mL, 0.59 mmol) and ethanesulfonamide (38 mg, 0.35 mmol) were added, after which the reaction mixture was kept at room temperature for 12 h. After confirmation of the completion of the reaction by TLC, the solution was evaporated to dryness, diluted with water and extracted with ethyl acetate. The combined organic layers were washed with sat. brine, dried over anhydrous sodium sulfate and evaporated in vacuo. Finally, column chromatography over silica gel (6-8% MeOH in CH₂Cl₂) allowed isolation of the envisaged compound as an off-white solid (15 mg, 10%).

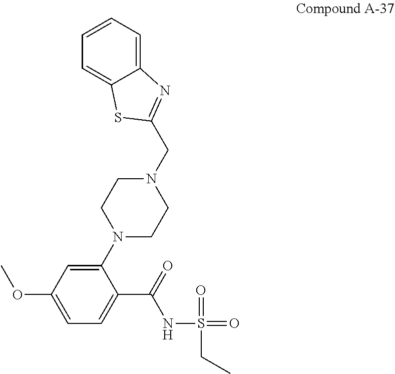 [SMILES: CCS(=O)(=O)NC(=O)c1ccc(OC)cc1N1CCN(Cc2nc3ccccc3s2)CC1]

Prepared through method S by adding iodomethane (0.49 mL, 0.78 mmol) to a solution of intermediate 18 (200 mg, 0.52 mmol) and K₂CO₃ (144 mg, 1.04 mmol) in DMF (30 mL). The envisaged compound was obtained after stirring the reaction mixture for 3 h at 80° C. After performing an aq. work-up and column chromatography (40-50 00 of ethyl acetate in hexane) methyl 2-(4-(benzo[d]thiazol-2-ylmethyl)piperazin-1-yl)-4-methoxybenzoate was obtained in good yield (156 mg, 750%).

Subsequent hydrolysis of the ester (150 mg, 0.38 mmol) in presence of lithium hydroxide monohydrate (18 mg, 0.75 mmol) in 10 mL of MeOH:H₂O (2:1) was completed after 4 h stirring at room temperature. Aq. work-up as described in method S delivered the crude acid (128 mg) which was used as such in the following reaction.

As such, mixing of the carboxylic acid (120 mg, 0.31 mmol) with CDI (101 mg, 0.63 mmol) in THE (10 mL) at 0° C., followed after 15 minutes by the addition of DBU (0.1 mL, 0.63 mmol) and ethanesulfonamide (41 mg, 0.38 mmol), afforded the envisaged end product after 12 h stirring of the reaction mixture at r.t. Aq. work-up, followed by column chromatography (5-6% o MeOH in CH₂Cl₂), yielded 2-[4-(1,3-benzothiazol-2-ylmethyl)piperazin-1-yl]-N-ethylsulfonyl-4-methoxy-benzamide as a pale-brown solid (7 mg, 5%).

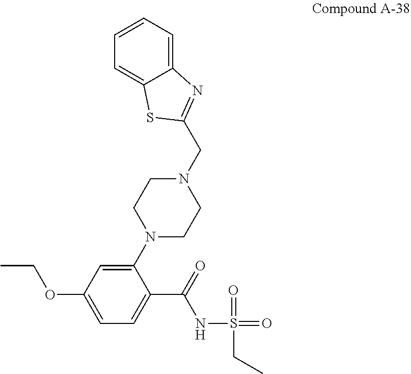 [SMILES: CCOc1ccc(C(=O)NS(=O)(=O)CC)c(N2CCN(Cc3nc4ccccc4s3)CC2)c1]

Prepared through method S by adding ethyl iodide (0.2 mL, 2.35 mmol) to a solution of intermediate 18 (600 mg, 1.56 mmol) and K₂CO₃ (432 mg, 3.13 mmol) in DMF (30 mL). The envisaged compound was obtained after stirring the reaction mixture for 4 h at 80° C. After performing an aq. work-up and column chromatography (45-50% of ethyl acetate in hexane) methyl 2-(4-(benzo[d]thiazol-2-ylmethyl)piperazin-1-yl)-4-ethoxybenzoate was obtained in excellent yield (582 mg, 90%).

Subsequent hydrolysis of the ester (580 mg, 1.41 mmol) in presence of lithium hydroxide monohydrate (68 mg, 2.82 mmol) in 30 mL of MeOH:H₂O (2:1) was completed after 5 h stirring at room temperature. Aq. work-up as described in method S delivered the crude acid (358 mg) which was used as such in the following reaction.

As such, mixing of the carboxylic acid (350 mg, 0.89 mmol) with CDI (285 mg, 1.76 mmol) in THE (20 mL) at 0° C., followed after 15 minutes by the addition of DBU (0.26 mL, 1.76 mmol) and ethanesulfonamide (115 mg, 1.06 mmol), afforded the envisaged end product after 12 h stirring of the reaction mixture at r.t. Aq. work-up, followed by column chromatography (5-6% MeOH in CH₂Cl₂), yielded 2-[4-(1,3-benzothiazol-2-ylmethyl)piperazin-1-yl]-4-ethoxy-N-ethylsulfonyl-benzamide as an off-white solid (16 mg, 4%).

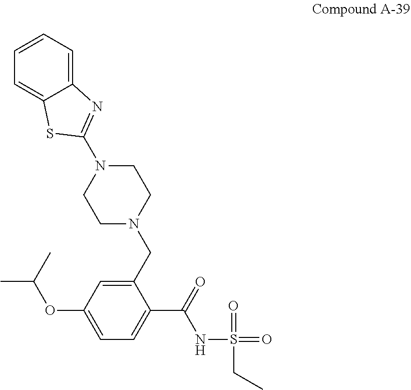 [SMILES: CCS(=O)(=O)NC(=O)c1ccc(OC(C)C)cc1CN1CCN(c2nc3ccccc3s2)CC1]

Prepared through method R from a solution of 2-(piperazin-1-yl)benzo[d]thiazole (0.23 g, 1.04 mmol) in DMF (30 mL), to which Et₃N (0.36 mL, 2.7 mmol) and methyl 2-(bromomethyl)-4-isopropoxybenzoate (0.25 g, 0.9 mmol) were added. The resulting reaction mixture was stirred at 100° C. for 12 h. Aq. work-up with EtOAc and column chromatography (2-3% MeOH in CH₂Cl₂) afforded methyl 2-((4-(benzo[d]thiazol-2-yl)piperazin-1-yl)methyl)-4-isopropoxybenzoate in moderate yield (163 mg, 44%).

Subsequent hydrolysis of the ester (150 mg, 0.35 mmol) in presence of lithium hydroxide monohydrate (17 mg, 0.70 mmol) in MeOH:H₂O (2:1) was completed after 3 h stirring at room temperature. Aq. work-up as described in method R delivered the crude acid (106 mg) which was used as such in the following reaction.

As such, mixing of the carboxylic acid (100 mg, 0.24 mmol) with CDI (79 mg, 0.48 mmol) in THE at 0° C., followed after 15 minutes by the addition of DBU (0.07 mL, 0.48 mmol) and ethanesulfonamide (32 mg, 0.29 mmol), afforded the envisaged end product after 12 h stirring of the reaction mixture at r.t. Aq. work-up, followed by column chromatography (6-8% MeOH in CH₂Cl₂), yielded 2-[[4-(1,3-benzothiazol-2-yl)piperazin-1-yl]methyl]-N-ethylsulfonyl-4-isopropoxy-benzamide as a pale-yellow solid (23 mg, 19%).

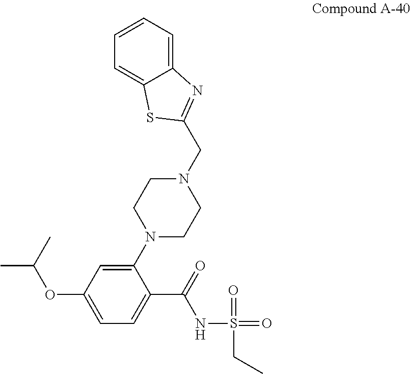 [SMILES: CCS(=O)(=O)NC(=O)c1ccc(OC(C)C)cc1N1CCN(Cc2nc3ccccc3s2)CC1]

Prepared through method Q by adding 2-(chloromethyl)benzo[d]thiazole (0.39 g, 2.15 mmol) to a solution of methyl 4-isopropoxy-2-(piperazin-1-yl)benzoate (0.5 g, 1.79 mmol) and Et₃N (0.62 mL, 4.49 mmol) in 1,4-dioxane (30 mL) to perform a nucleophilic substitution. The envisaged compound was obtained after stirring the reaction mixture for 3 h at 80° C. After performing an aq. work-up and column chromatography (30-40% of ethyl acetate in hexane) methyl 2-(4-(benzo[d]thiazol-2-ylmethyl)piperazin-1-yl)-4-isopropoxybenzoate was obtained in moderate yield (322 mg, 42%).

Subsequent hydrolysis of the ester (300 mg, 0.70 mmol) in presence of lithium hydroxide monohydrate (34 mg, 1.41 mmol) in MeOH:H₂O (2:1) was completed after 3 h stirring at room temperature. Aq. work-up as described in method Q delivered the crude acid which was used as such in the following reaction.

As such, mixing of the carboxylic acid (150 mg, 0.36 mmol) with CDI (118 mg, 0.73 mmol) in THE at 0° C., followed after 15 minutes by the addition of DBU (0.10 mL, 0.73 mmol) and ethanesulfonamide (48 mg, 0.43 mmol), afforded the envisaged end product after 15 h stirring of the reaction mixture at r.t. Aq. work-up, followed by column chromatography (6-8% MeOH in CH₂Cl₂), yielded 2-[4-(1,3-benzothiazol-2-ylmethyl)piperazin-1-yl]-N-ethylsulfonyl-4-isopropoxy-benzamide as an off-white solid (13 mg, 7%).

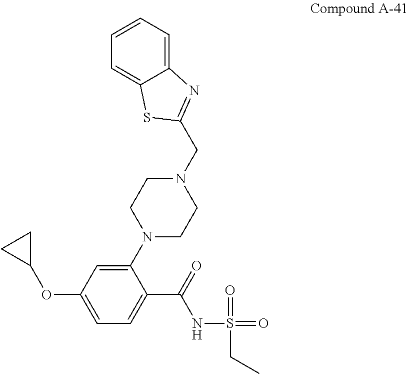 [SMILES: CCS(=O)(=O)NC(=O)c1ccc(OC2CC2)cc1N1CCN(Cc2nc3ccccc3s2)CC1]

Prepared through method S by adding bromocyclopropane (0.06 mL, 0.78 mmol) to a solution of intermediate 18 (200 mg, 0.52 mmol) and K₂CO₃ (144 mg, 1.04 mmol) in DMF (10 mL). The envisaged compound was obtained after stirring the reaction mixture for 6 h at 80° C. After performing an aq. work-up and column chromatography (40-45% of ethyl acetate in hexane) methyl 2-(4-(benzo[d]thiazol-2-ylmethyl)piperazin-1-yl)-4-cyclopropoxybenzoate was obtained in moderate yield (106 mg, 48%).

Subsequent hydrolysis of the ester (100 mg, 0.24 mmol) in presence of lithium hydroxide monohydrate (11 mg, 0.48 mmol) in 8 mL of MeOH:H₂O (2:1) was completed after 5 h stirring at room temperature. Aq. work-up as described in method S delivered the crude acid (63 mg) which was used as such in the following reaction.

As such, mixing of the carboxylic acid (60 mg, 0.15 mmol) with CDI (48 mg, 0.29 mmol) in THE (8 mL) at 0° C., followed after 15 minutes by the addition of DBU (0.044 mL, 0.29 mmol) and ethanesulfonamide (19 mg, 0.18 mmol), afforded the envisaged end product after 14 h stirring of the reaction mixture at r.t. Aq. work-up, followed by column chromatography (6-8% MeOH in CH₂Cl₂), yielded 2-[4-(1,3-benzothiazol-2-ylmethyl)piperazin-1-yl]-4-(cyclopropoxy)-N-ethylsulfonyl-benzamide as an off-white solid (6 mg, 8%).

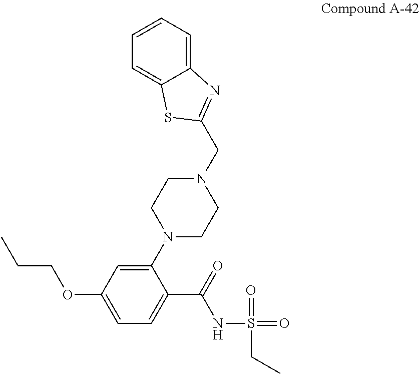 [SMILES: CCCOc1ccc(C(=O)NS(=O)(=O)CC)c(N2CCN(Cc3nc4ccccc4s3)CC2)c1]

Prepared through method Q by adding 2-(chloromethyl)benzo[d]thiazole (0.40 g, 2.15 mmol) to a solution of methyl 2-(piperazin-1-yl)-4-propoxybenzoate (0.5 g, 1.79 mmol) and Et₃N (0.62 mL, 4.49 mmol) in 1,4-dioxane (30 mL) to perform a nucleophilic substitution. The envisaged compound was obtained after stirring the reaction mixture for 3 h at 80° C. After performing an aq. work-up and column chromatography (35-40% of ethyl acetate in hexane) methyl 2-(4-(benzo[d]thiazol-2-ylmethyl)piperazin-1-yl)-4-propoxybenzoate was obtained in moderate yield (376 mg, 49%).

Subsequent hydrolysis of the ester (370 mg, 0.87 mmol) in presence of lithium hydroxide monohydrate (42 mg, 1.74 mmol) in MeOH:H₂O (2:1) was completed after 6 h stirring at room temperature. Aq. work-up as described in method Q delivered the crude acid which was used as such in the following reaction.

As such, mixing of the carboxylic acid (200 mg, 0.49 mmol) with CDI (158 mg, 0.97 mmol) in THE at 0° C., followed after 15 minutes by the addition of DBU (0.15 mL, 0.97 mmol) and ethanesulfonamide (64 mg, 0.58 mmol), afforded the envisaged end product after 14 h stirring of the reaction mixture at r.t. Aq. work-up, followed by column chromatography (6-8% MeOH in CH₂Cl₂), yielded 2-[4-(1,3-benzothiazol-2-ylmethyl)piperazin-1-yl]-N-ethylsulfonyl-4-propoxy-benzamide as an off-white solid (36 mg, 15%).

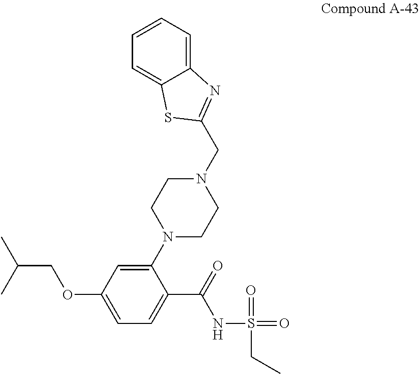 [SMILES: CCS(=O)(=O)NC(=O)c1ccc(OCC(C)C)cc1N1CCN(Cc2nc3ccccc3s2)CC1]

Prepared through method S by adding 1-bromo-2-methylpropane (0.26 mL, 2.35 mmol) to a solution of intermediate 18 (600 mg, 1.56 mmol) and K₂CO₃ (432 mg, 3.13 mmol) in DMF (35 mL). The envisaged compound was obtained after stirring the reaction mixture for 8 h at 80° C. After performing an aq. work-up and column chromatography (40-50% of ethyl acetate in hexane) methyl 2-(4-(benzo[d]thiazol-2-ylmethyl)piperazin-1-yl)-4-isobutoxybenzoate was obtained in good yield (545 mg, 79%).

Subsequent hydrolysis of the ester (540 mg, 1.23 mmol) in presence of lithium hydroxide monohydrate (59 mg, 2.46 mmol) in 30 mL of MeOH:H₂O (2:1) was completed after 4 h stirring at room temperature. Aq. work-up as described in method S delivered the crude acid (278 mg) which was used as such in the following reaction.

As such, mixing of the carboxylic acid (270 mg, 0.63 mmol) with CDI (206 mg, 1.27 mmol) in THE (20 mL) at 0° C., followed after 15 minutes by the addition of DBU (0.19 mL, 1.27 mmol) and ethanesulfonamide (83 mg, 0.76 mmol), afforded the envisaged end product after 12 h stirring of the reaction mixture at r.t. Aq. work-up, followed by column chromatography (7-8% MeOH in CH₂Cl₂), yielded 2-[4-(1,3-benzothiazol-2-ylmethyl)piperazin-1-yl]-N-ethylsulfonyl-4-isobutoxy-benzamide as an off-white solid (16 mg, 5%).

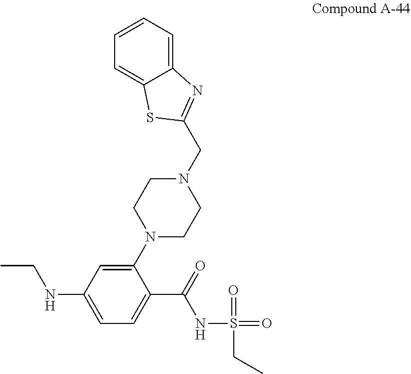 [SMILES: CCNc1ccc(C(=O)NS(=O)(=O)CC)c(N2CCN(Cc3nc4ccccc4s3)CC2)c1]

Prepared through method T. To a stirred solution of intermediate 3 (300 mg, 0.67 mmol) in 1,4-dioxane (25 mL) was added ethylamine (45 mg, 1.01 mmol), followed by K₃PO₄ (285 mg, 1.34 mmol) and P(tBu)₃·BF₄ (194 mg, 0.67 mmol). The resultant mixture was bubbled with argon for 20 min, after which Pd(dba)₂ (62 mg, 0.07 mmol) and BINAP (89 mg, 0.14 mmol) were added. Upon completion of the addition, the reaction was brought to 100° C. for 12 h. The reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (4-5% MeOH in CH₂Cl₂), yielded the compound of interest as a gummy solid (214 mg, 78%).

Subsequent hydrolysis of the ester (200 mg, 0.49 mmol) in presence of lithium hydroxide monohydrate (23 mg, 0.97 mmol) in MeOH:H₂O (15 mL, 2:1 ratio) was completed after 6 h stirring at room temperature. Aq. work-up as described in method T delivered the crude acid (123 mg crude) which was used as such in the following reaction.

To a solution of the carboxylic acid (120 mg, 0.30 mmol) in THE (15 mL) was added CDI (98 mg, 0.60 mmol) at 0° C., after which the reaction was left stirring at room temperature for 15 minutes. Next, DBU (0.09 mL, 0.60 mmol) and ethanesulfonamide (40 mg, 0.36 mmol) were added, after which the reaction mixture was kept at room temperature for 14 h. After confirmation of the completion of the reaction by TLC, the solution was evaporated to dryness, water was added followed by extraction with ethyl acetate. The combined organic layers were washed with sat. brine, dried over anhydrous sodium sulfate and evaporated in vacuo. Finally, column chromatography over silica gel (6-8% MeOH in CH₂Cl₂) allowed isolation of the envisaged compound as a pale-yellow solid (8 mg, 5%).

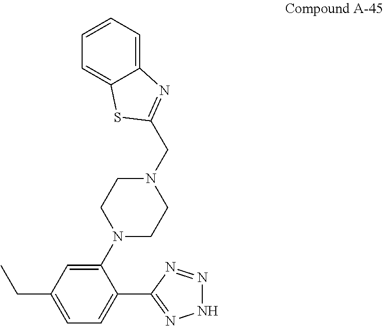 [SMILES: CCc1ccc(-c2nn[nH]n2)c(N2CCN(Cc3nc4ccccc4s3)CC2)c1]

Prepared through method Y. To a stirred solution of intermediate 19 (300 mg, 0.73 mmol) in 1,4-dioxane (30 mL) was added ethylboronic acid (64 mg, 0.87 mmol), followed by K₂CO₃ (250 mg, 1.82 mmol) and the resultant mixture was bubbled with argon for 20 min. Then Pd(dppf)Cl₂ (53 mg, 0.073 mmol) was added after which the reaction was heated to 80° C. for 8 h. The reaction mixture was evaporated under reduced pressure and the residue was re-dissolved with ethyl acetate and washed with water and sat. brine. Subsequent drying over anhydrous sodium sulfate and evaporation in vacuo, followed by column chromatography over silica gel (30-40% EtOAc in hexane) afforded the targeted compound in good yield (182 mg, 69%).

Final tetrazole reaction was performed, by mixing the nitrile (180 mg, 0.50 mmol), NaN₃ (258 mg, 3.97 mmol) and Bu₃SnCl (1.08 mL, 3.97 mmol) in toluene (15 mL) at 140° C. for 15 h in a sealed tube. Aq. work-up as described in method Y, followed by column chromatography (SiO₂, 4-5% MeOH in CH₂Cl₂) and final trituration using diethyl ether, yielded the desired tetrazole as an off-white solid (19 mg, 10%).

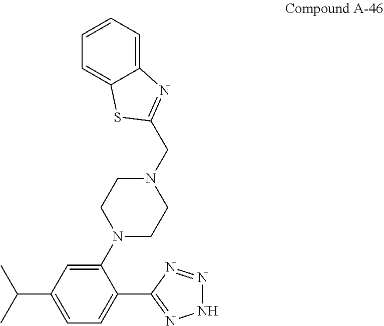 [SMILES: CC(C)c1ccc(-c2nn[nH]n2)c(N2CCN(Cc3nc4ccccc4s3)CC2)c1]

Prepared through method Y. To a stirred solution of intermediate 19 (300 mg, 0.73 mmol) in 1,4-dioxane (30 mL) was added 4,4,5,5-tetramethyl-2-(prop-1-en-2-yl)-1,3,2-dioxaborolane (146 mg, 0.87 mmol), followed by K₂CO₃ (250 mg, 1.82 mmol) and the resultant mixture was bubbled with argon for 20 min. Then Pd(dppf)Cl₂ (53 mg, 0.073 mmol) was added after which the reaction was heated to 80° C. for 6 h. The reaction mixture was evaporated in vacuo to remove the volatiles and the residue was re-dissolved with ethyl acetate and washed with water and sat. brine. Subsequent drying over anhydrous sodium sulfate and evaporation under reduced pressure, followed by column chromatography over silica gel (30-40% EtOAc in hexane) yielded the desired compound in good yield (232 mg, 85%).

A solution of 2-(4-(benzo[d]thiazol-2-ylmethyl)piperazin-1-yl)-4-(prop-1-en-2-yl)benzonitrile (230 mg, 0.61 mmol) in MeOH (20 mL) was hydrogenated over 10% Pd/C (23 mg) under 5 Kg/cm² H₂ pressure using a Parr hydrogenator for 3 h at ambient temperature. The reaction mixture was filtered over a Celite Bed® and concentrated under reduced pressure. The obtained crude compound was used as such in the next step (182 mg crude).

Final tetrazole reaction was performed, by mixing the nitrile (180 mg, 0.48 mmol), NaN₃ (249 mg, 3.82 mmol) and Bu₃SnCl (0.96 mL, 3.82 mmol) in toluene (15 mL) at 140° C. for 16 h in a sealed tube. Aq. work-up, followed by column chromatography (SiO₂, 4-5% MeOH in CH₂Cl₂) and final trituration using diethyl ether, afforded the envisaged tetrazole as an off-white solid (21 mg, 11%).

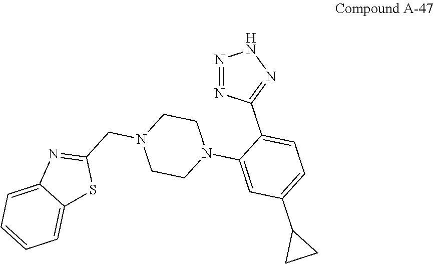 [SMILES: c1ccc2sc(CN3CCN(c4cc(C5CC5)ccc4-c4nn[nH]n4)CC3)nc2c1]

Prepared through method Y. To a stirred solution of intermediate 19 (300 mg, 0.73 mmol) in 1,4-dioxane (30 mL) was added cyclopropylboronic acid (187 mg, 2.18 mmol), followed by K₂CO₃ (250 mg, 1.82 mmol) and the resultant mixture was bubbled with argon for 20 min. Then Pd(dppf)Cl₂ (53 mg, 0.073 mmol) was added after which the reaction was heated to 80° C. for 10 h. The reaction mixture was concentrated under reduced pressure to remove the volatiles and the residue was re-dissolved with ethyl acetate and washed with water and sat. brine. Subsequent drying over anhydrous sodium sulfate and evaporation under reduced pressure, followed by column chromatography over silica gel (30-40% EtOAc in hexane) afforded the desired compound in modest yield (105 mg, 39%).

Final tetrazole reaction was performed, by mixing the nitrile (100 mg, 0.27 mmol), NaN₃ (139 mg, 2.14 mmol) and Bu₃SnCl (0.53 mL, 2.14 mmol) in toluene (10 mL) at 140° C. for 18 h in a sealed tube. Aq. work-up as described in method Y, followed by column chromatography (SiO₂, 4-5% MeOH in CH₂Cl₂) and final trituration using diethyl ether, yielded the envisaged compound A-47 as a pale-brown solid (9 mg, 8%).

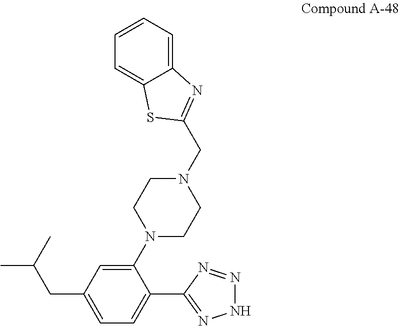 [SMILES: CC(C)Cc1ccc(-c2nn[nH]n2)c(N2CCN(Cc3nc4ccccc4s3)CC2)c1]

Prepared through method U from a solution of intermediate 1 (200 mg, 0.71 mmol) in DMF (15 mL), to which Et₃N (0.40 mL, 2.86 mmol) and 2-(chloromethyl)benzo[d]thiazole (156 mg, 0.86 mmol) were added. The resulting reaction mixture was stirred at 60° C. for 10 h. Aq. work-up with EtOAc and column chromatography (2-3% MeOH in CH₂Cl₂) afforded 2-(4-(benzo[d]thiazol-2-ylmethyl)piperazin-1-yl)-4-isobutylbenzonitrile in excellent yield (234 mg, 84%).

Final tetrazole reaction was performed, by mixing the nitrile (230 mg, 0.59 mmol), NaN₃ (306 mg, 4.71 mmol) and Bu₃SnCl (1.18 mL, 4.71 mmol) in toluene (20 mL) at 150° C. for 24 h in a sealed tube. Aq. work-up, followed by column chromatography (SiO₂, 5-6% MeOH in CH₂Cl₂) and final trituration using diethyl ether, afforded the envisaged tetrazole as a pale-yellow solid (42 mg, 16%).

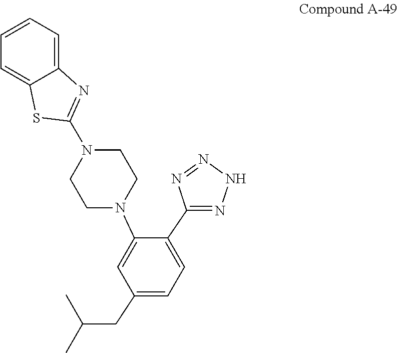 [SMILES: CC(C)Cc1ccc(-c2nn[nH]n2)c(N2CCN(c3nc4ccccc4s3)CC2)c1]

Prepared through method U from a solution of intermediate 1 (200 mg, 0.71 mmol) in DMF (15 mL), to which Et₃N (0.40 mL, 2.86 mmol) and 2-chlorobenzo[d]thiazole (145 mg, 0.86 mmol) were added. The resulting reaction mixture was stirred at 80° C. for 12 h. Aq. work-up with EtOAc and column chromatography (3-4% MeOH in CH₂Cl₂) afforded 2-(4-(benzo[d]thiazol-2-yl)piperazin-1-yl)-4-isobutylbenzonitrile in modest yield (114 mg, 43%).

Final tetrazole reaction was performed, by mixing the nitrile (110 mg, 0.29 mmol), NaN₃ (152 mg, 2.33 mmol) and Bu₃SnCl (0.58 mL, 2.33 mmol) in toluene (10 mL) at 150° C. for 24 h in a sealed tube. Aq. work-up, followed by column chromatography (SiO₂, 6-8% MeOH in CH₂Cl₂) and final trituration using diethyl ether, afforded the desired tetrazole as a pale-yellow solid (11 mg, 9%).

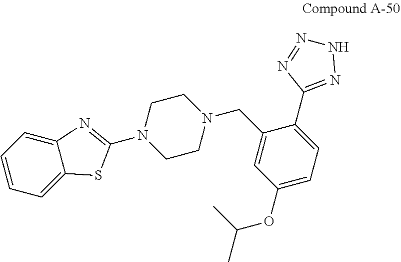 [SMILES: CC(C)Oc1ccc(-c2nn[nH]n2)c(CN2CCN(c3nc4ccccc4s3)CC2)c1]

Prepared through method H from a solution of 2-(piperazin-1-yl)benzo[d]thiazole (0.16 g, 0.71 mmol) in DMF (30 mL), to which Et₃N (0.25 mL, 1.8 mmol) and 2-(bromomethyl)-4-isopropoxybenzonitrile (0.15 g, 0.6 mmol) were added. The resulting reaction mixture was stirred at 100° C. for 3 h. Aq. work-up with EtOAc and column chromatography (2-3% MeOH in CH₂Cl₂) afforded 2-((4-(benzo[d]thiazol-2-yl)piperazin-1-yl)methyl)-4-isopropoxybenzonitrile in moderate yield (68 mg, 28%).

Final tetrazole reaction was performed, by mixing the nitrile (60 mg, 0.15 mmol), NaN₃ (79 mg, 1.22 mmol) and Bu₃SnCl (0.30 mL, 1.22 mmol) in toluene (5 mL) at 150° C. for 24 h in a sealed tube. Aq. work-up, followed by column chromatography (SiO₂, 5-6% MeOH in CH₂Cl₂), afforded the envisaged tetrazole as an off-white solid (8 mg, 12%).

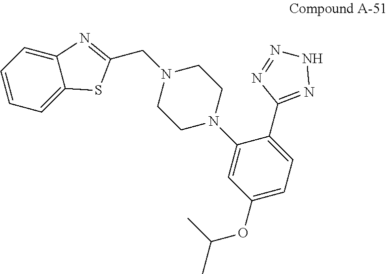 [SMILES: CC(C)Oc1ccc(-c2nn[nH]n2)c(N2CCN(Cc3nc4ccccc4s3)CC2)c1]

Prepared through method C from 4-isopropoxy-2-(piperazin-1-yl)benzonitrile (intermediate 14, 0.50 g, 2.04 mmol) and 2-(chloromethyl)benzo[d]thiazole (0.41 g, 2.24 mmol). The alkylation reaction was completed after 3 h at 80° C. After performing an aq. work-up, the organic residue was purified by silica chromatography (2-3% MeOH in CH₂Cl₂) to afford 2-(4-(benzo[d]thiazol-2-ylmethyl)piperazin-1-yl)-4-isopropoxybenzonitrile (360 mg, 45%).

Final tetrazole reaction by mixing the nitrile (100 mg, 0.25 mmol), NaN₃ (132 mg, 2.03 mmol) and Bu₃SnCl (0.51 mL, 2.03 mmol) in toluene (6 mL) at 150° C. for 24 h in a sealed tube, followed by aq. work-up and column chromatography (5-6% MeOH in CH₂Cl₂), afforded the desired tetrazole as an off-white solid (7 mg, 7%).

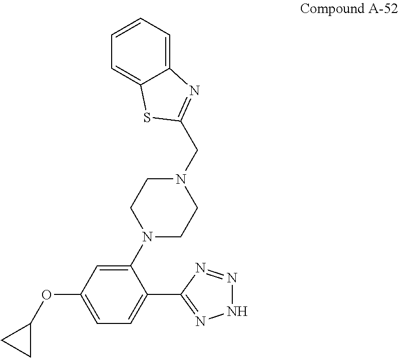 [SMILES: c1ccc2sc(CN3CCN(c4cc(OC5CC5)ccc4-c4nn[nH]n4)CC3)nc2c1]

Prepared through method Z by adding bromocyclopropane (124 mg, 1.03 mmol) to a solution of intermediate 20 (300 mg, 0.86 mmol) and Cs₂CO₃ (558 mg, 1.71 mmol) in NMP (20 mL). The target compound was obtained after stirring the reaction mixture for 12 h at 140° C. After performing an aq. work-up and column chromatography (3-4% of MeOH in CH₂Cl₂) 2-(4-(benzo[d]thiazol-2-ylmethyl)piperazin-1-yl)-4-cyclopropoxybenzonitrile was obtained in moderate yield (141 mg, 42%).

Final tetrazole reaction was performed, by mixing the nitrile (130 mg, 0.33 mmol), NaN₃ (173 mg, 2.66 mmol) and Bu₃SnCl (0.67 mL, 2.66 mmol) in toluene (15 mL) at 140° C. for 20 h in a sealed tube. Aq. work-up as described in method Z, followed by column chromatography (SiO₂, 6-8% MeOH in CH₂Cl₂) and final trituration using diethyl ether, afforded 2-[[4-[5-(cyclopropoxy)-2-(2H-tetrazol-5-yl)phenyl]piperazin-1-yl]methyl]-1,3-benzothiazole as an off-white solid (12 mg, 8%).

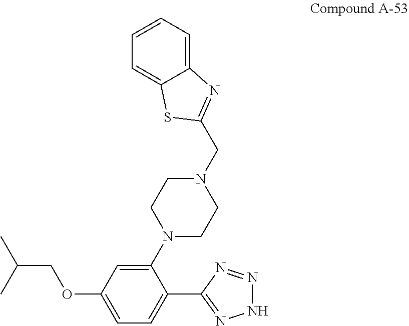 [SMILES: CC(C)COc1ccc(-c2nn[nH]n2)c(N2CCN(Cc3nc4ccccc4s3)CC2)c1]

Prepared through method Z by adding 1-bromo-2-methylpropane (117 mg, 0.86 mmol) to a solution of intermediate 20 (250 mg, 0.71 mmol) and K₂CO₃ (197 mg, 1.43 mmol) in DMF (15 mL). The envisaged compound was obtained after stirring the reaction mixture for 3 h at 80° C. After performing an aq. work-up as described in method Z and column chromatography (2-3% of MeOH in CH₂Cl₂) 2-(4-(benzo[d]thiazol-2-ylmethyl)piperazin-1-yl)-4-isobutoxybenzonitrile was obtained in good yield (245 mg, 84%).

Final tetrazole reaction was performed, by mixing the nitrile (240 mg, 0.59 mmol), NaN₃ (307 mg, 4.72 mmol) and Bu₃SnCl (1.18 mL, 4.72 mmol) in toluene (15 mL) at 140° C. for 16 h in a sealed tube. Aq. work-up, followed by column chromatography (SiO₂, 7-8% MeOH in CH₂Cl₂) and final trituration using diethyl ether, afforded the targeted tetrazole as a pale-brown solid (10 mg, 4%).

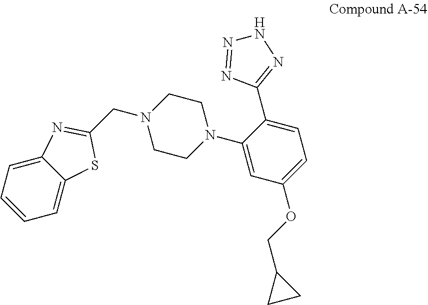 [SMILES: c1ccc2sc(CN3CCN(c4cc(OCC5CC5)ccc4-c4nn[nH]n4)CC3)nc2c1]

Prepared through method Z by adding (bromomethyl)cyclopropane (231 mg, 1.71 mmol) to a solution of intermediate 20 (500 mg, 1.43 mmol) and K₂CO₃ (394 mg, 2.85 mmol) in DMF (25 mL). 2-(4-(Benzo[d]thiazol-2-ylmethyl)piperazin-1-yl)-4-(cyclopropylmethoxy)benzonitrile was obtained after stirring the reaction mixture for 8 h at 80° C. After performing an aq. work-up and column chromatography (3-5% of MeOH in CH₂Cl₂) the targeted nitrile was obtained in good yield (468 mg, 81%).

Final tetrazole reaction was performed, by mixing the nitrile (450 mg, 1.08 mmol), NaN₃ (560 mg, 8.60 mmol) and Bu₃SnCl (2.15 mL, 8.60 mmol) in toluene (25 mL) at 140° C. for 15 h in a sealed tube. Aq. work-up as described in method Z, followed by column chromatography (SiO₂, 6-7% MeOH in CH₂Cl₂) and trituration using diethyl ether, delivered the targeted final compound as an off-white solid (21 mg, 4%).

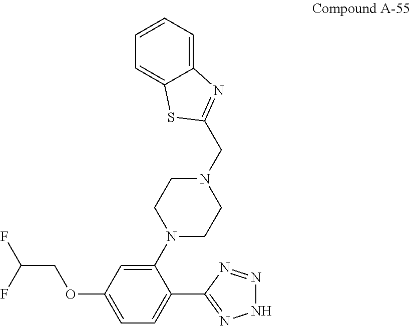 [SMILES: FC(F)COc1ccc(-c2nn[nH]n2)c(N2CCN(Cc3nc4ccccc4s3)CC2)c1]

Prepared through method Z by adding 2-bromo-1,1-difluoroethane (199 mg, 1.37 mmol) to a solution of intermediate 20 (400 mg, 1.14 mmol) and K₂CO₃ (316 mg, 2.28 mmol) in DMF (20 mL). The envisaged compound was obtained after stirring the reaction mixture for 4 h at 80° C. After performing an aq. work-up and column chromatography (2-3% of MeOH in CH₂Cl₂) 2-(4-(benzo[d]thiazol-2-ylmethyl)piperazin-1-yl)-4-(2,2-difluoroethoxy)benzonitrile was obtained in excellent yield (452 mg, 95%).

Final tetrazole reaction was performed, by mixing the nitrile (450 mg, 1.09 mmol), NaN₃ (565 mg, 8.69 mmol) and Bu₃SnCl (2.17 mL, 8.69 mmol) in toluene (20 mL) at 140° C. for 14 h in a sealed tube. Aq. work-up as described in method Z, followed by column chromatography (SiO₂, 7-8% MeOH in CH₂Cl₂) and ultimate trituration in diethyl ether, afforded the desired final compound as a pale-yellow solid (21 mg, 4%).

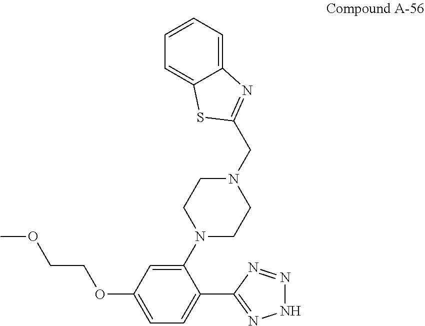 [SMILES: COCCOc1ccc(-c2nn[nH]n2)c(N2CCN(Cc3nc4ccccc4s3)CC2)c1]

Prepared through method Z by adding 1-bromo-2-methoxyethane (143 mg, 1.03 mmol) to a solution of intermediate 20 (300 mg, 0.86 mmol) and K₂CO₃ (237 mg, 1.71 mmol) in DMF (20 mL). The targeted nitrile was obtained after stirring the reaction mixture for 8 h at 80° C. After performing an aq. work-up and column chromatography (3-5% of MeOH in CH₂Cl₂), as described in method Z, 2-(4-(benzo[d]thiazol-2-ylmethyl)piperazin-1-yl)-4-(2-methoxyethoxy)benzonitrile was obtained in good yield (243 mg, 69%).

Final tetrazole reaction was performed, by mixing the nitrile (240 mg, 0.59 mmol), NaN₃ (306 mg, 4.70 mmol) and Bu₃SnCl (1.17 mL, 4.70 mmol) in toluene (20 mL) at 140° C. for 16 h in a sealed tube. Aq. work-up, followed by column chromatography (SiO₂, 8-10% MeOH in CH₂Cl₂) and final trituration in diethyl ether, afforded the envisaged tetrazole as a pale-brown solid (8 mg, 3%).

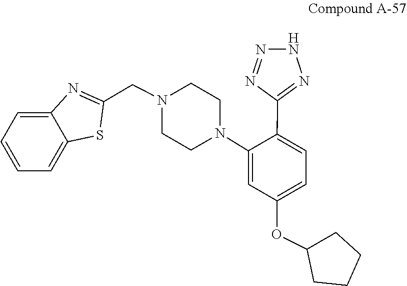 [SMILES: c1ccc2sc(CN3CCN(c4cc(OC5CCCC5)ccc4-c4nn[nH]n4)CC3)nc2c1]

Prepared through method Z by adding bromocyclopentane (255 mg, 1.71 mmol) to a solution of intermediate 20 (500 mg, 1.43 mmol) and K₂CO₃ (394 mg, 2.85 mmol) in DMF (25 mL). 2-(4-(Benzo[d]thiazol-2-ylmethyl)piperazin-1-yl)-4-(cyclopentyloxy)benzonitrile was obtained after stirring the reaction mixture for 6 h at 80° C. Performing an aq. work-up as described in method Z and column chromatography (3-4% of MeOH in CH₂Cl₂) afforded the envisaged nitrile in good yield (458 mg, 77%).

Final tetrazole reaction was performed, by mixing the nitrile (450 mg, 1.08 mmol), NaN₃ (560 mg, 8.60 mmol) and Bu₃SnCl (2.15 mL, 8.60 mmol) in toluene (20 mL) at 140° C. for 18 h in a sealed tube. Aq. work-up, followed by column chromatography (SiO₂, 7-8% MeOH in CH₂Cl₂) and final trituration in diethyl ether, yielded the desired tetrazole as an off-white solid (22 mg, 4%).

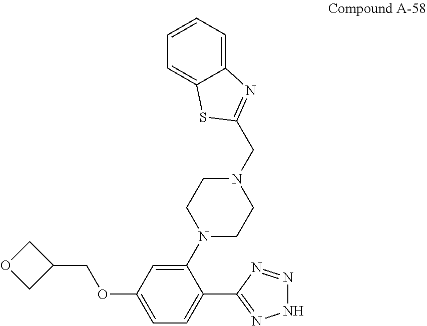 [SMILES: c1ccc2sc(CN3CCN(c4cc(OCC5COC5)ccc4-c4nn[nH]n4)CC3)nc2c1]

Prepared through method Z by adding 3-(iodomethyl)oxetane (163 mg, 0.82 mmol) to a solution of intermediate 20 (240 mg, 0.68 mmol) and K₂CO₃ (189 mg, 1.37 mmol) in DMF (15 mL). The targeted nitrile was obtained after stirring the reaction mixture for 12 h at 80° C. Performing an aq. work-up as described in method Z and column chromatography (4-5% of MeOH in CH₂Cl₂) afforded 2-(4-(benzo[d]thiazol-2-ylmethyl)piperazin-1-yl)-4-(oxetan-3-ylmethoxy)benzonitrile in moderate yield (122 mg, 42%).

Final tetrazole reaction was performed, by mixing the nitrile (120 mg, 2.85 mmol), NaN₃ (148 mg, 2.28 mmol) and Bu₃SnCl (0.57 mL, 2.28 mmol) in toluene (15 mL) at 140° C. for 14 h in a sealed tube. Aq. work-up as described in method Z, followed by column chromatography (SiO₂, 7-8% MeOH in CH₂Cl₂) and ultimate trituration in diethyl ether, yielded the envisaged compound A-58 as an off-white solid (5 mg, 4%).

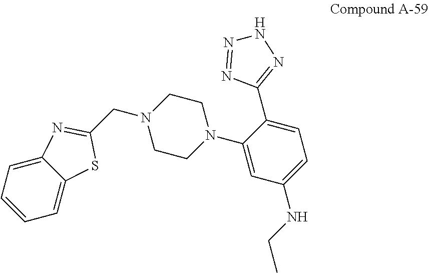 [SMILES: CCNc1ccc(-c2nn[nH]n2)c(N2CCN(Cc3nc4ccccc4s3)CC2)c1]

Prepared through method AA. To a stirred solution of intermediate 19 (300 mg, 0.73 mmol) in 1,4-dioxane (20 mL) was added ethylamine (49 mg, 1.09 mmol), followed by NaOtBu (105 mg, 1.09 mmol) and P(tBu)₃·BF₄ (316 mg, 1.09 mmol). The resultant mixture was bubbled with argon for 20 min, after which Pd(dba)₂ (42 mg, 0.07 mmol) and BINAP (90 mg, 0.15 mmol) were added. Upon completion of the addition, the reaction was brought to 100° C. for 12 h. The reaction mixture was concentrated under reduced pressure to remove the volatiles and the residue was re-dissolved with ethyl acetate after which an extraction with water and sat. brine was performed. Subsequent drying over anhydrous sodium sulfate and evaporation in vacuo, followed by column chromatography over silica gel (4-5% MeOH in CH₂Cl₂), yielded the nitrile of interest as a gummy liquid (257 mg, 94%).

Final tetrazole reaction was performed, by mixing the nitrile (250 mg, 0.66 mmol), NaN₃ (344 mg, 5.30 mmol) and Bu₃SnCl (1.32 mL, 5.30 mmol) in toluene (20 mL) at 140° C. for 18 h in a sealed tube. Aq. work-up as described in method AA, followed by column chromatography (SiO₂, 8-10% MeOH in CH₂Cl₂) and trituration in diethyl ether, yielded the envisaged substituted tetrazole as an off-white solid (17 mg, 6%).

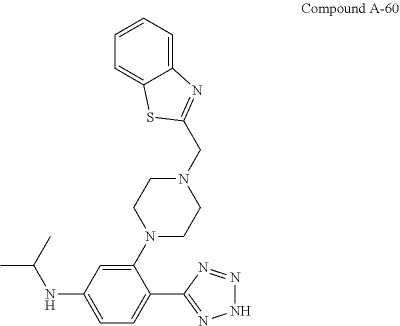 [SMILES: CC(C)Nc1ccc(-c2nn[nH]n2)c(N2CCN(Cc3nc4ccccc4s3)CC2)c1]

Prepared through method AA. To a stirred solution of intermediate 19 (250 mg, 0.60 mmol) in 1,4-dioxane (20 mL) was added isopropylamine (54 mg, 0.91 mmol), followed by NaOtBu (87 mg, 0.91 mmol) and P(tBu)₃·BF₄ (263 mg, 0.91 mmol). The resultant mixture was bubbled with argon for 20 min, after which Pd(dba)₂ (35 mg, 0.06 mmol) and BINAP (75 mg, 0.12 mmol) were added. Upon completion of the addition, the reaction was brought to 100° C. for 12 h. The reaction mixture was evaporated in vacuo to remove the volatiles and the residue was re-dissolved with ethyl acetate after which the combined organic layers were washed with water and sat. brine. Subsequent drying over anhydrous sodium sulfate and evaporation under reduced pressure, followed by column chromatography over silica gel (3-5% MeOH in CH₂Cl₂), yielded the compound of interest as a gummy solid (206 mg, 87%).

Final tetrazole reaction was performed, by mixing the nitrile (200 mg, 0.51 mmol), NaN₃ (266 mg, 4.09 mmol) and Bu₃SnCl (1.02 mL, 4.09 mmol) in toluene (15 mL) at 140° C. for 16 h in a sealed tube. Aq. work-up as described in method AA, followed by column chromatography (SiO₂, 9-10% MeOH in CH₂Cl₂) and final trituration in diethyl ether, yielded 3-[4-(1,3-benzothiazol-2-ylmethyl)piperazin-1-yl]-N-isopropyl-4-(2H-tetrazol-5-yl)aniline as an off-white solid (14 mg, 6%).

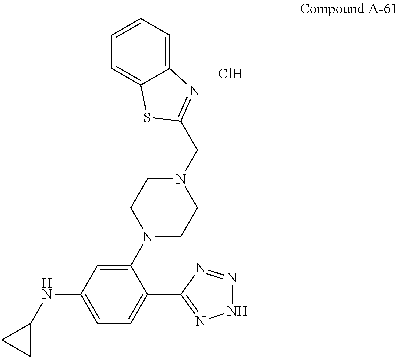 [SMILES: c1ccc2sc(CN3CCN(c4cc(NC5CC5)ccc4-c4nn[nH]n4)CC3)nc2c1 ; Cl]

Prepared through method AA. To a stirred solution of intermediate 19 (500 mg, 1.21 mmol) in 1,4-dioxane (40 mL) was added cyclopropylamine (104 mg, 1.81 mmol), followed by NaOtBu (174 mg, 1.81 mmol) and P(tBu)₃·BF₄ (526 mg, 1.81 mmol). The resultant mixture was bubbled with argon for 20 min, after which Pd(dba)₂ (70 mg, 0.12 mmol) and BINAP (151 mg, 0.24 mmol) were added. Upon completion of the addition, the reaction was brought to 100° C. for 14 h. The reaction mixture was worked up as described in method AA. Subsequent column chromatography over silica gel (5-6% MeOH in CH₂Cl₂) yielded 2-(4-(benzo[d]thiazol-2-ylmethyl)piperazin-1-yl)-4-(cyclopropylamino)benzonitrile as a gummy liquid (413 mg, 90%).

Prior to the tetrazole formation an additional Boc protection was performed. Therefore, to a stirred solution of 2-(4-(benzo[d]thiazol-2-ylmethyl)piperazin-1-yl)-4-(cyclopropylamino)benzonitrile (400 mg, 1.03 mmol) in 1,2-dichloroethane (10 mL) at 0° C. was added Et₃N (0.28 mL, 2.05 mmol). The solution was stirred at 0° C. for 10 minutes, after which Boc-anhydride (268 mg, 1.23 mmol) and a catalytic amount of DMAP were added. The reaction was brought to 90° C. and kept stirring at 90° C. for 4 hours. After completion of the reaction was confirmed by TLC, the reaction mixture was concentrated in vacuo and the obtained residue was re-dissolved in ethyl acetate. An extraction with water and sat. brine was performed. Subsequent drying over anhydrous sodium sulfate and evaporation under reduced pressure afforded a crude residue, which was further purified by column chromatography over silica gel (2-3% EtOAc in hexane). tert-Butyl(3-(4-(benzo[d]thiazol-2-ylmethyl)piperazin-1-yl)-4-cyanophenyl)(cyclopropyl)carbamate was isolated as a gummy solid in excellent yield (482 mg, 94%).

Next, the tetrazole reaction was performed, by mixing the nitrile (480 mg, 0.98 mmol), NaN₃ (510 mg, 7.84 mmol) and Bu₃SnCl (1.96 mL, 7.84 mmol) in toluene (25 mL) at 140° C. for 22 h in a sealed tube. Aq. work-up as described in method AA, followed by column chromatography (SiO₂, 7-9% MeOH in CH₂Cl₂) and trituration in diethyl ether, yielded the envisaged tetrazole as a pale-yellow solid (63 mg, 12%).

Final Boc deprotection through addition of HCl (g) in dioxane (7 mL) to a solution of the tetrazole containing intermediate (60 mg, 0.11 mmol) in 1,4-dioxane (3 mL) at 0° C. was performed. The resulting mixture was stirred at r.t. for 3 h. Upon completion of the reaction, the volatiles were removed in vacuo and final trituration with hexane delivered the targeted hydrochloride salt as a pale-yellow solid (10 mg, 20%).

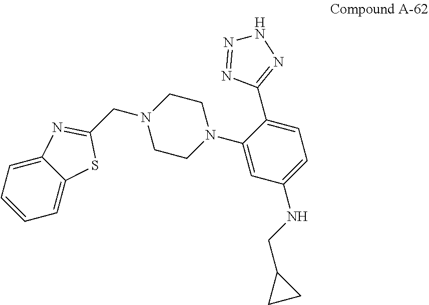 [SMILES: c1ccc2sc(CN3CCN(c4cc(NCC5CC5)ccc4-c4nn[nH]n4)CC3)nc2c1]

Prepared through method AA. To a stirred solution of intermediate 19 (250 mg, 0.60 mmol) in 1,4-dioxane (20 mL) was added cyclopropylmethanamine (65 mg, 0.90 mmol), followed by NaOtBu (87 mg, 0.91 mmol) and P(tBu)₃·BF₄ (263 mg, 0.91 mmol). The resultant mixture was bubbled with argon for 20 min, after which Pd(dba)₂ (35 mg, 0.06 mmol) and BINAP (75 mg, 0.12 mmol) were added. Upon completion of the addition, the reaction was brought to 100° C. for 10 h. The reaction mixture was concentrated under reduced pressure to remove the volatiles and the residue was re-dissolved with ethyl acetate after which an extraction with water and sat. brine was performed. Subsequent drying over anhydrous sodium sulfate and evaporation under reduced pressure, followed by column chromatography over silica gel (4-6% MeOH in CH₂Cl₂), afforded the intermediate nitrile of interest as a gummy solid (207 mg, 85%).

Final tetrazole reaction was performed, by mixing the nitrile (200 mg, 0.50 mmol), NaN₃ (258 mg, 3.97 mmol) and Bu₃SnCl (1.0 mL, 3.97 mmol) in toluene (20 mL) at 140° C. for 16 h in a sealed tube. Aq. work-up as described in method AA, followed by column chromatography (SiO₂, 8-10% MeOH in CH₂Cl₂) and subsequent trituration in diethyl ether, yielded the envisaged tetrazole as an off-white solid (16 mg, 7%).

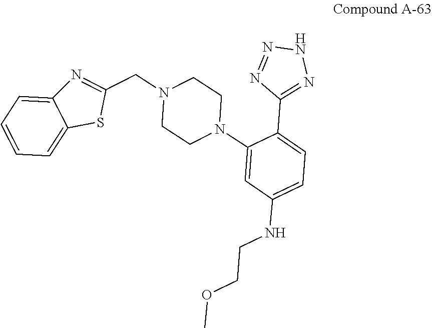 [SMILES: COCCNc1ccc(-c2nn[nH]n2)c(N2CCN(Cc3nc4ccccc4s3)CC2)c1]

Prepared through method AA. To a stirred solution of intermediate 19 (250 mg, 0.60 mmol) in 1,4-dioxane (20 mL) was added 2-methoxyethanamine (68 mg, 0.91 mmol), followed by NaOtBu (87 mg, 0.91 mmol) and P(tBu)₃·BF₄ (263 mg, 0.91 mmol). The resultant mixture was bubbled with argon for 20 min, after which Pd(dba)₂ (35 mg, 0.06 mmol) and BINAP (75 mg, 0.12 mmol) were added. Upon completion of the addition, the reaction was brought to 100° C. for 14 h. Performing an aq. work-up as described in method AA, followed by column chromatography over silica gel (5-6% MeOH in CH₂Cl₂), yielded 2-(4-(benzo[d]thiazol-2-ylmethyl)piperazin-1-yl)-4-((2-methoxyethyl)amino)benzonitrile as a gummy liquid (177 mg, 72%).

Final tetrazole reaction was performed, by mixing the nitrile (170 mg, 0.42 mmol), NaN₃ (216 mg, 3.33 mmol) and Bu₃SnCl (0.83 mL, 3.33 mmol) in toluene (20 mL) at 140° C. for 22 h in a sealed tube. Aq. work-up as described in method AA, followed by column chromatography (SiO₂, 9-10% MeOH in CH₂Cl₂) and subsequent trituration in diethyl ether, afforded the desired tetrazole as an off-white solid (6 mg, 3%).

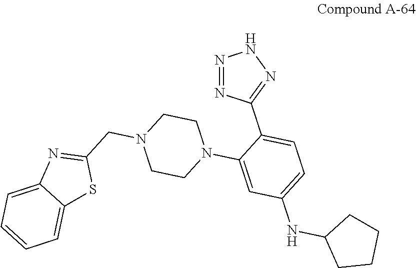 [SMILES: c1ccc2sc(CN3CCN(c4cc(NC5CCCC5)ccc4-c4nn[nH]n4)CC3)nc2c1]

Prepared through method AA. To a stirred solution of intermediate 19 (250 mg, 0.60 mmol) in 1,4-dioxane (20 mL) was added cyclopentylamine (77 mg, 0.90 mmol), followed by NaOtBu (87 mg, 0.91 mmol) and P(tBu)₃·BF₄ (263 mg, 0.91 mmol). The resultant mixture was bubbled with argon for 20 min, after which Pd(dba)₂ (35 mg, 0.06 mmol) and BINAP (75 mg, 0.12 mmol) were added. Upon completion of the addition, the reaction was brought to 100° C. for 14 h. The reaction mixture was concentrated in vacuo to remove the volatiles and the residue was re-dissolved with ethyl acetate after which an extraction with water and sat. brine was performed. Subsequent drying over anhydrous sodium sulfate and evaporation under reduced pressure, followed by column chromatography over silica gel (5-6% MeOH in CH₂Cl₂), afforded the desired intermediate nitrile as a gummy liquid (164 mg, 65%).

Final tetrazole reaction was performed, by mixing the nitrile (160 mg, 0.38 mmol), NaN₃ (200 mg, 3.07 mmol) and Bu₃SnCl (0.77 mL, 3.07 mmol) in toluene (15 mL) at 140° C. for 22 h in a sealed tube. Aq. work-up as described in method AA, followed by column chromatography (SiO₂, 7-8% MeOH in CH₂Cl₂) and final trituration in diethyl ether, delivered the envisaged compound A-64 as an off-white solid (15 mg, 9%).

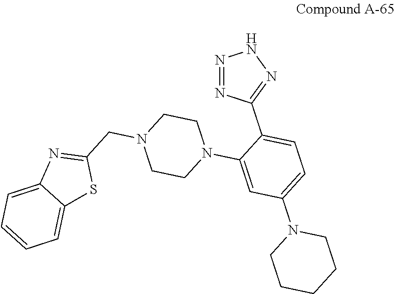 [SMILES: c1ccc2sc(CN3CCN(c4cc(N5CCCCC5)ccc4-c4nn[nH]n4)CC3)nc2c1]

Prepared through method AA. To a stirred solution of intermediate 19 (300 mg, 0.73 mmol) in 1,4-dioxane (20 mL) was added piperidine (93 mg, 1.09 mmol), followed by NaOtBu (105 mg, 1.09 mmol) and P(tBu)₃·BF₄ (316 mg, 1.09 mmol). The resultant mixture was bubbled with argon for 20 min, after which Pd(dba)₂ (42 mg, 0.07 mmol) and BINAP (90 mg, 0.15 mmol) were added. Upon completion of the addition, the reaction was brought to 100° C. for 12 h. The reaction mixture was worked up as described in method AA. Subsequent column chromatography over silica gel (4-5% MeOH in CH₂Cl₂), afforded the envisaged nitrile as a gummy solid (257 mg, 85%).

Final tetrazole reaction was performed, by mixing the nitrile (250 mg, 0.60 mmol), NaN₃ (311 mg, 4.79 mmol) and Bu₃SnCl (1.20 mL, 4.79 mmol) in toluene (20 mL) at 140° C. for 22 h in a sealed tube. Aq. work-up as described in method AA, followed by column chromatography (SiO₂, 7-8% MeOH in CH₂Cl₂) and subsequent trituration with diethyl ether, delivered 2-((4-(5-(piperidin-1-yl)-2-(2H-tetrazol-5-yl)phenyl)piperazin-1-yl)methyl) benzo[d]thiazole as an off-white solid (22 mg, 8%).

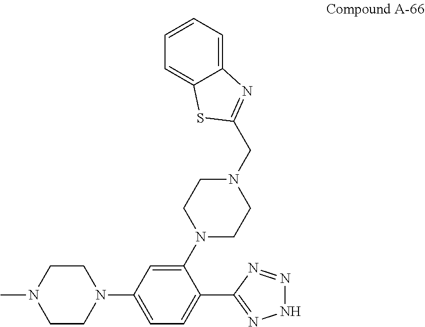 [SMILES: CN1CCN(c2ccc(-c3nn[nH]n3)c(N3CCN(Cc4nc5ccccc5s4)CC3)c2)CC1]

Prepared through method AA. To a stirred solution of intermediate 19 (300 mg, 0.73 mmol) in 1,4-dioxane (20 mL) was added 1-methylpiperazine (109 mg, 1.09 mmol), followed by NaOtBu (105 mg, 1.09 mmol) and P(tBu)₃·BF₄ (316 mg, 1.09 mmol). The resultant mixture was bubbled with argon for 20 min, after which Pd(dba)₂ (42 mg, 0.07 mmol) and BINAP (90 mg, 0.15 mmol) were added. Upon completion of the addition, the reaction was brought to 100° C. for 15 h. The reaction mixture was evaporated under reduced pressure to remove the volatiles and the residue was re-dissolved with ethyl acetate after which an extraction with water and sat. brine was performed. Subsequent drying over anhydrous sodium sulfate and concentration in vacuo, followed by column chromatography over silica gel (4-5% MeOH in CH₂Cl₂), afforded the desired substituted nitrile as a gummy liquid (253 mg, 81%).

Final tetrazole reaction was performed, by mixing the nitrile (250 mg, 0.58 mmol), NaN₃ (300 mg, 4.62 mmol) and Bu₃SnCl (1.16 mL, 4.62 mmol) in toluene (20 mL) at 140° C. for 24 h in a sealed tube. Aq. work-up as described in method AA, followed by column chromatography (SiO₂, 7-8% MeOH in CH₂Cl₂) and final trituration in diethyl ether, delivered the targeted substituted tetrazole as a pale-brown solid (4 mg, 2%).

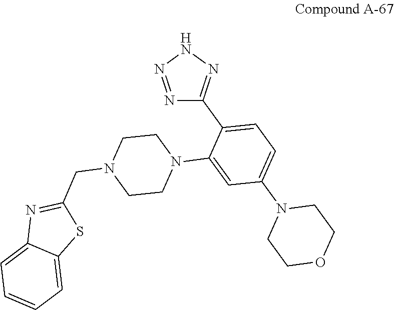 [SMILES: c1ccc2sc(CN3CCN(c4cc(N5CCOCC5)ccc4-c4nn[nH]n4)CC3)nc2c1]

Prepared through method AA. To a stirred solution of intermediate 19 (300 mg, 0.73 mmol) in 1,4-dioxane (20 mL) was added morpholine (95 mg, 1.09 mmol), followed by NaOtBu (105 mg, 1.09 mmol) and P(tBu)₃·BF₄ (316 mg, 1.09 mmol). The resultant mixture was bubbled with argon for 20 min, after which Pd(dba)₂ (42 mg, 0.07 mmol) and BINAP (90 mg, 0.15 mmol) were added. Upon completion of the addition, the reaction was brought to 100° C. for 14 h. The reaction mixture was concentrated in vacuo to remove the volatiles and the residue was re-dissolved with ethyl acetate after which an extraction with water and sat. brine was performed. Subsequent drying over anhydrous sodium sulfate and evaporation under reduced pressure, followed by column chromatography over silica gel (4-5% MeOH in CH₂Cl₂), delivered the envisaged nitrile as a gummy solid (271 mg, 89%).

Final tetrazole reaction was performed, by mixing the nitrile (250 mg, 0.60 mmol), NaN₃ (310 mg, 4.77 mmol) and Bu₃SnCl (1.19 mL, 4.77 mmol) in toluene (10 mL) at 140° C. for 24 h in a sealed tube. Aq. work-up as described in method AA, followed by column chromatography (SiO₂, 6-8% MeOH in CH₂Cl₂) and ultimate trituration in diethyl ether, afforded the desired compound A-67 as a pale-yellow solid (23 mg, 8%).

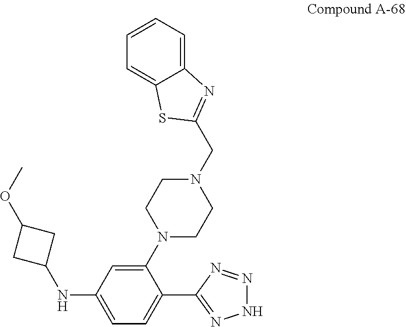 [SMILES: COC1CC(Nc2ccc(-c3nn[nH]n3)c(N3CCN(Cc4nc5ccccc5s4)CC3)c2)C1]

Prepared through method AA. To a stirred solution of intermediate 19 (375 mg, 0.91 mmol) in 1,4-dioxane (25 mL) was added 3-methoxycyclobutanamine hydrochloride (187 mg, 1.36 mmol), followed by NaOtBu (174 mg, 1.81 mmol) and P(tBu)₃·BF₄ (526 mg, 1.81 mmol). The resultant mixture was bubbled with argon for 20 min, after which Pd(dba)₂ (52 mg, 0.09 mmol) and BINAP (113 mg, 0.18 mmol) were added. Upon completion of the addition, the reaction was brought to 100° C. for 15 h. Performing an aq. work-up as described in method AA, followed by column chromatography over silica gel (5-6% MeOH in CH₂Cl₂), yielded the envisaged intermediate nitrile as a gummy liquid (320 mg, 81%).

Final tetrazole reaction was performed, by mixing the nitrile (200 mg, 0.46 mmol), NaN₃ (240 mg, 3.70 mmol) and Bu₃SnCl (0.92 mL, 3.70 mmol) in toluene (20 mL) at 140° C. for 24 h in a sealed tube. Aq. work-up as described in method AA, followed by column chromatography (SiO₂, 8-10% MeOH in CH₂Cl₂) and subsequent trituration in diethyl ether, afforded the desired substituted tetrazole as an off-white solid (8 mg, 4%).

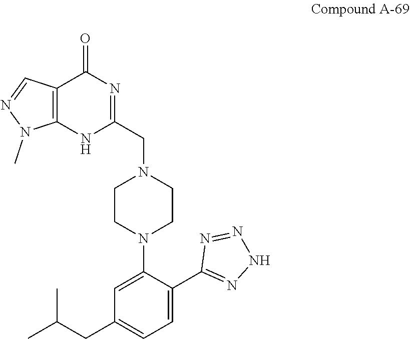 [SMILES: CC(C)Cc1ccc(-c2nn[nH]n2)c(N2CCN(Cc3nc(=O)c4cnn(C)c4[nH]3)CC2)c1]

Prepared through method U from a solution of intermediate 1 (100 mg, 0.36 mmol) in DMF (10 mL), to which Et₃N (0.20 mL, 1.43 mmol) and 6-(chloromethyl)-1-methyl-7H-pyrazolo[3,4-d]pyrimidin-4-one (85 mg, 0.43 mmol) were added. The resulting reaction mixture was stirred at 60° C. for 12 h. Aq. work-up with EtOAc and column chromatography (5-7% MeOH in CH₂Cl₂) afforded 4-isobutyl-2-(4-((1-methyl-4-oxo-4,7-dihydro-1H-pyrazolo[3,4-d]pyrimidin-6-yl)methyl)piperazin-1-yl)benzonitrile in moderate yield (78 mg, 54%).

Final tetrazole reaction was performed, by mixing the nitrile (75 mg, 0.18 mmol), NaN₃ (96 mg, 1.48 mmol) and Bu₃SnCl (0.37 mL, 1.48 mmol) in toluene (10 mL) at 140° C. for 24 h in a sealed tube. Aq. work-up, followed by column chromatography (SiO₂, 8-10% MeOH in CH₂Cl₂) and final trituration using diethyl ether, afforded the targeted tetrazole as a colorless solid (13 mg, 16%).

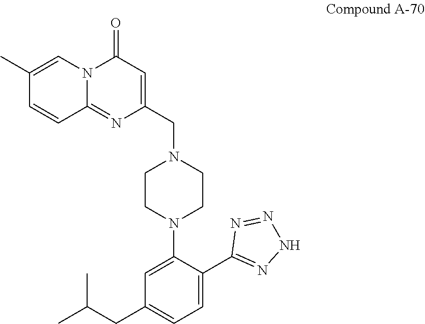 [SMILES: Cc1ccc2nc(CN3CCN(c4cc(CC(C)C)ccc4-c4nn[nH]n4)CC3)cc(=O)n2c1]

Prepared through method U from a solution of intermediate 1 (100 mg, 0.36 mmol) in DMF (10 mL), to which Et₃N (0.20 mL, 1.43 mmol) and 2-(chloromethyl)-7-methyl-pyrido[1,2-a]pyrimidin-4-one (89 mg, 0.43 mmol) were added. The resulting reaction mixture was stirred at 60° C. for 8 h. Aq. work-up with EtOAc and column chromatography (4-5% MeOH in CH₂Cl₂) afforded 4-isobutyl-2-(4-((7-methyl-4-oxo-4H-pyrido[1,2-a]pyrimidin-2-yl)methyl)piperazin-1-yl)benzonitrile in moderate yield (88 mg, 59%).

Final tetrazole reaction was performed, by mixing the nitrile (85 mg, 0.20 mmol), NaN₃ (106 mg, 1.60 mmol) and Bu₃SnCl (0.41 mL, 1.60 mmol) in toluene (10 mL) at 140° C. for 16 h in a sealed tube. Aq. work-up, followed by column chromatography (SiO₂, 8-10% MeOH in CH₂Cl₂) and final trituration using diethyl ether, yielded the envisaged tetrazole as an off-white solid (19 mg, 20%).

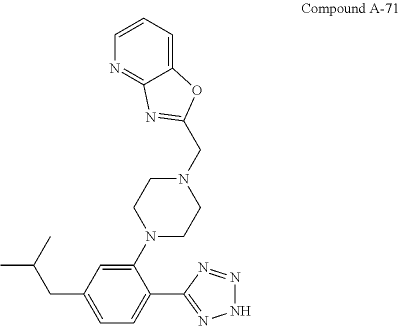 [SMILES: CC(C)Cc1ccc(-c2nn[nH]n2)c(N2CCN(Cc3nc4ncccc4o3)CC2)c1]

Prepared through method U from a solution of intermediate 1 (100 mg, 0.36 mmol) in DMF (10 mL), to which Et₃N (0.20 mL, 1.43 mmol) and 2-(chloromethyl)oxazolo[4,5-b]pyridine (72 mg, 0.43 mmol) were added. The resulting reaction mixture was stirred at 60° C. for 12 h. Aq. work-up with EtOAc and column chromatography (4-5% MeOH in CH₂Cl₂) afforded 4-isobutyl-2-(4-(oxazolo[4,5-b]pyridin-2-ylmethyl)piperazin-1-yl)benzonitrile in good yield (83 mg, 62%).

Final tetrazole reaction was performed, by mixing the nitrile (80 mg, 0.21 mmol), NaN₃ (110 mg, 1.70 mmol) and Bu₃SnCl (0.43 mL, 1.70 mmol) in toluene (10 mL) at 140° C. for 18 h in a sealed tube. Aq. work-up, as described in method U, followed by column chromatography (SiO₂, 6-8% MeOH in CH₂Cl₂) and final trituration using diethyl ether, yielded the envisaged tetrazole as a pale-yellow solid (7 mg, 8%).

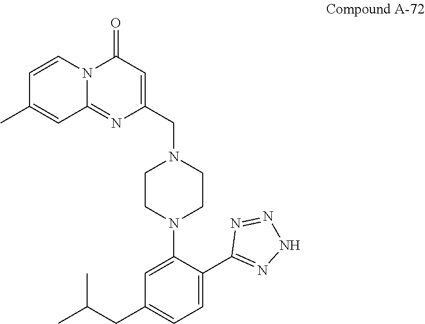 [SMILES: Cc1ccn2c(=O)cc(CN3CCN(c4cc(CC(C)C)ccc4-c4nn[nH]n4)CC3)nc2c1]

Prepared through method U from a solution of intermediate 1 (100 mg, 0.36 mmol) in DMF (10 mL), to which Et₃N (0.20 mL, 1.43 mmol) and 2-(chloromethyl)-8-methyl-pyrido[1,2-a]pyrimidin-4-one (89 mg, 0.43 mmol) were added. The resulting reaction mixture was stirred at 60° C. for 7 h. Aq. work-up with EtOAc and column chromatography (3-5% MeOH in CH₂Cl₂) afforded 4-isobutyl-2-(4-((8-methyl-4-oxo-4H-pyrido[1,2-a]pyrimidin-2-yl)methyl)piperazin-1-yl)benzonitrile in good yield (98 mg, 66%).

Final tetrazole reaction was performed, by mixing the nitrile (95 mg, 0.23 mmol), NaN₃ (119 mg, 1.83 mmol) and Bu₃SnCl (0.46 mL, 1.83 mmol) in toluene (10 mL) at 140° C. for 14 h in a sealed tube. Aq. work-up, as described in method U, followed by column chromatography (SiO₂, 8-10% MeOH in CH₂Cl₂) and final trituration using diethyl ether, yielded 2-[[4-[5-isobutyl-2-(2H-tetrazol-5-yl)phenyl]piperazin-1-yl]methyl]-8-methyl-pyrido[1,2-a]pyrimidin-4-one as a pale-yellow solid (17 mg, 19%).

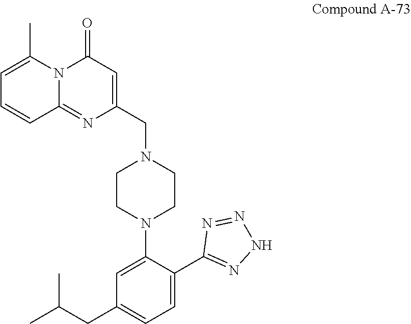 [SMILES: Cc1cccc2nc(CN3CCN(c4cc(CC(C)C)ccc4-c4nn[nH]n4)CC3)cc(=O)n12]

Prepared through method U from a solution of intermediate 1 (130 mg, 0.46 mmol) in DMF (15 mL), to which Et₃N (0.26 mL, 1.86 mmol) and 2-(chloromethyl)-6-methyl-pyrido[1,2-a]pyrimidin-4-one (116 mg, 0.56 mmol) were added. The resulting reaction mixture was stirred at 60° C. for 8 h. Aq. work-up with EtOAc and column chromatography (5-6% MeOH in CH₂Cl₂) afforded 4-isobutyl-2-(4-((6-methyl-4-oxo-4H-pyrido[1,2-a]pyrimidin-2-yl)methyl)piperazin-1-yl)benzonitrile in good yield (145 mg, 76%).

Final tetrazole reaction was performed, by mixing the nitrile (140 mg, 0.34 mmol), NaN₃ (175 mg, 2.70 mmol) and Bu₃SnCl (0.67 mL, 2.70 mmol) in toluene (10 mL) at 140° C. for 16 h in a sealed tube. Aq. work-up, as described in method U, followed by column chromatography (SiO₂, 8-10% MeOH in CH₂Cl₂) and final trituration using diethyl ether, afforded the targeted tetrazole as a pale-yellow solid (4 mg, 3%).

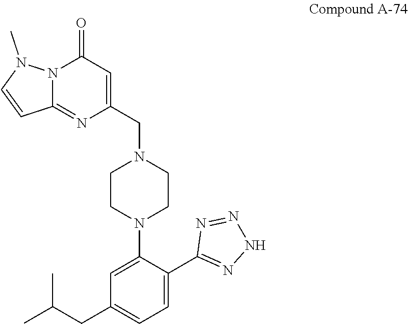 [SMILES: CC(C)Cc1ccc(-c2nn[nH]n2)c(N2CCN(Cc3cc(=O)n4c(ccn4C)n3)CC2)c1]

Prepared through method U from a solution of intermediate 1 (150 mg, 0.54 mmol) in DMF (10 mL), to which Et₃N (0.30 mL, 2.14 mmol) and 5-(chloromethyl)-1-methyl-pyrazolo[1,5-a]pyrimidin-7-one (127 mg, 0.64 mmol) were added. The resulting reaction mixture was stirred at 60° C. for 10 h. Aq. work-up with EtOAc and column chromatography (4-6% MeOH in CH₂Cl₂) afforded 4-isobutyl-2-(4-((1-methyl-7-oxo-1,7-dihydropyrazolo[1,5-a]pyrimidin-5-yl)methyl)piperazin-1-yl)benzonitrile in modest yield (63 mg, 29%).

Final tetrazole reaction was performed, by mixing the nitrile (60 mg, 0.15 mmol), NaN₃ (77 mg, 1.18 mmol) and Bu₃SnCl (0.3 mL, 1.18 mmol) in toluene (10 mL) at 140° C. for 20 h in a sealed tube. Aq. work-up, as described in method U, followed by column chromatography (SiO₂, 9-10% MeOH in CH₂Cl₂) and final trituration using diethyl ether, afforded the envisaged compound as a pale-brown solid (6 mg, 9%).

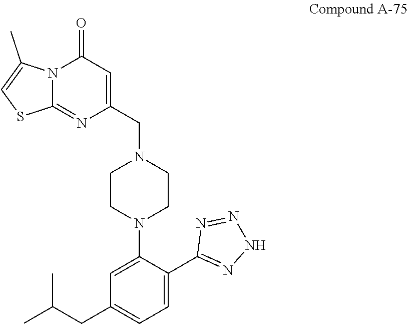 [SMILES: Cc1csc2nc(CN3CCN(c4cc(CC(C)C)ccc4-c4nn[nH]n4)CC3)cc(=O)n12]

Prepared through method U from a solution of intermediate 1 (120 mg, 0.43 mmol) in DMF (10 mL), to which Et₃N (0.24 mL, 1.71 mmol) and 7-(chloromethyl)-3-methyl-thiazolo[3,2-a]pyrimidin-5-one (110 mg, 0.51 mmol) were added. The resulting reaction mixture was stirred at 60° C. for 10 h. Aq. work-up with EtOAc and column chromatography (5-6% MeOH in CH₂Cl₂) afforded 4-isobutyl-2-(4-((3-methyl-5-oxo-5H-thiazolo[3,2-a]pyrimidin-7-yl)methyl)piperazin-1-yl)benzonitrile in good yield (113 mg, 62%).

Final tetrazole reaction was performed, by mixing the nitrile (110 mg, 0.26 mmol), NaN₃ (136 mg, 2.09 mmol) and Bu₃SnCl (0.52 mL, 2.09 mmol) in toluene (15 mL) at 140° C. for 22 h in a sealed tube. Aq. work-up, as described in method U, followed by column chromatography (SiO₂, 8-10% MeOH in CH₂Cl₂) and final trituration using diethyl ether, afforded 7-[[4-[5-isobutyl-2-(2H-tetrazol-5-yl)phenyl]piperazin-1-yl]methyl]-3-methyl-thiazolo[3,2-a]pyrimidin-5-one as a pale-yellow solid (17 mg, 14%).

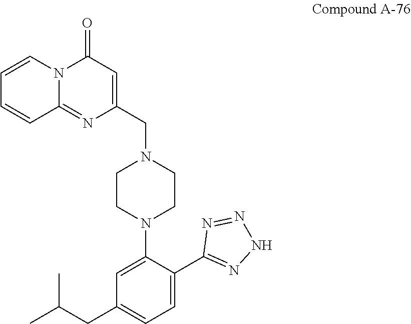 [SMILES: CC(C)Cc1ccc(-c2nn[nH]n2)c(N2CCN(Cc3cc(=O)n4ccccc4n3)CC2)c1]

Prepared through method U from a solution of intermediate 1 (200 mg, 0.71 mmol) in DMF (15 mL), to which Et₃N (0.40 mL, 2.86 mmol) and 2-(chloromethyl)pyrido[1,2-a]pyrimidin-4-one (167 mg, 0.86 mmol) were added. The resulting reaction mixture was stirred at 60° C. for 8 h. Aq. work-up with EtOAc and column chromatography (3-5% MeOH in CH₂Cl₂) afforded 4-isobutyl-2-(4-((4-oxo-4H-pyrido[1,2-a]pyrimidin-2-yl)methyl)piperazin-1-yl)benzonitrile in good yield (168 mg, 59%).

Final tetrazole reaction was performed, by mixing the nitrile (160 mg, 0.40 mmol), NaN₃ (207 mg, 3.19 mmol) and Bu₃SnCl (0.8 mL, 3.19 mmol) in toluene (10 mL) at 140° C. for 16 h in a sealed tube. Aq. work-up, as described in method U, followed by column chromatography (SiO₂, 7-8% MeOH in CH₂Cl₂) and final trituration using diethyl ether, afforded the desired tetrazole as an off-white solid (16 mg, 9%).

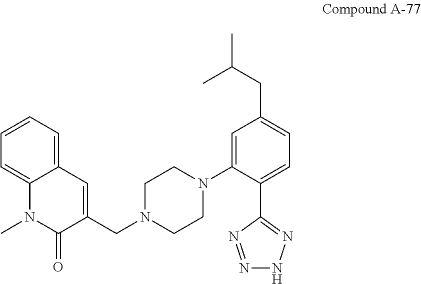 [SMILES: CC(C)Cc1ccc(-c2nn[nH]n2)c(N2CCN(Cc3cc4ccccc4n(C)c3=O)CC2)c1]

Prepared through method U from a solution of intermediate 1 (265 mg, 0.95 mmol) in DMF (15 mL), to which Et₃N (0.52 mL, 3.79 mmol) and 3-(chloromethyl)-1-methyl-quinolin-2-one (237 mg, 1.14 mmol) were added. The resulting reaction mixture was stirred at 60° C. for 8 h. Aq. work-up with EtOAc and column chromatography (3-4% MeOH in CH₂Cl₂) afforded 4-isobutyl-2-(4-((1-methyl-2-oxo-1,2-dihydroquinolin-3-yl)methyl)piperazin-1-yl)benzonitrile in good yield (262 mg, 67%).

Final tetrazole reaction was performed, by mixing the nitrile (260 mg, 0.63 mmol), NaN₃ (326 mg, 5.02 mmol) and Bu₃SnCl (1.25 mL, 5.02 mmol) in toluene (8 mL) at 140° C. for 16 h in a sealed tube. Aq. work-up, as described in method U, followed by column chromatography (SiO₂, 5-6% MeOH in CH₂Cl₂) and final trituration using diethyl ether, yielded the targeted tetrazole as a pale-yellow solid (22 mg, 8%).

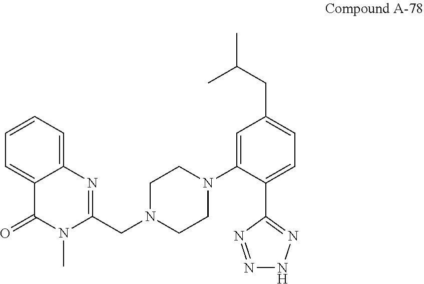 [SMILES: CC(C)Cc1ccc(-c2nn[nH]n2)c(N2CCN(Cc3nc4ccccc4c(=O)n3C)CC2)c1]

Prepared through method U from a solution of intermediate 1 (200 mg, 0.71 mmol) in DMF (15 mL), to which Et₃N (0.40 mL, 2.86 mmol) and 2-(chloromethyl)-3-methyl-quinazolin-4-one (179 mg, 0.86 mmol) were added. The resulting reaction mixture was stirred at 60° C. for 10 h. Aq. work-up with EtOAc and column chromatography (3-4% MeOH in CH₂Cl₂) afforded 4-isobutyl-2-(4-((3-methyl-4-oxo-3,4-dihydroquinazolin-2-yl)methyl)piperazin-1-yl)benzonitrile in excellent yield (273 mg, 92%).

Final tetrazole reaction was performed, by mixing the nitrile (270 mg, 0.65 mmol), NaN₃ (338 mg, 5.20 mmol) and Bu₃SnCl (1.30 mL, 5.20 mmol) in toluene (10 mL) at 140 5° C. for 15 h in a sealed tube. Aq. work-up, as described in method U, followed by column chromatography (SiO₂, 7-8% MeOH in CH₂Cl₂) and final trituration using diethyl ether, yielded the targeted compound as an off-white solid (16 mg, 5%).

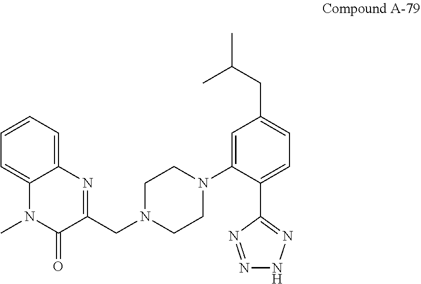 [SMILES: CC(C)Cc1ccc(-c2nn[nH]n2)c(N2CCN(Cc3nc4ccccc4n(C)c3=O)CC2)c1]

Prepared through method U from a solution of intermediate 1 (200 mg, 0.71 mmol) in DMF (15 mL), to which Et₃N (0.40 mL, 2.86 mmol) and 3-(chloromethyl)-1H-quinoxalin-2-one (167 mg, 0.86 mmol) were added. The resulting reaction mixture was stirred at r.t. for 12 h. Aq. work-up with EtOAc and column chromatography (4-5% MeOH in CH₂Cl₂) afforded 4-isobutyl-2-(4-((3-oxo-3,4-dihydroquinoxalin-2-yl)methyl)piperazin-1-yl)benzonitrile in moderate yield (152 mg, 53%).

Next, a methylation reaction was performed. To a stirred solution of 4-isobutyl-2-(4-((3-oxo-3,4-dihydroquinoxalin-2-yl)methyl)piperazin-1-yl)benzonitrile (150 mg, 0.37 mmol) in THE (10 mL) at 0° C. was added DBU (0.08 mL, 0.56 mmol), followed after stirring for 10 minutes at r.t. by the addition of methyl iodide (0.03 mL, 0.45 mmol). The reaction was kept stirring at room temperature for 4 h until completion of the reaction was confirmed by TLC. Next, the reaction mixture was evaporated to dryness, diluted with cold water and extraction with EtOAc was performed. The combined organic layers were washed with sat. brine, dried over anhydrous sodium sulfate and concentrated in vacuo. The obtained residual crude was purified by column chromatography over silica gel (2-3% MeOH in CH₂Cl₂) to yield 4-isobutyl-2-(4-((4-methyl-3-oxo-3,4-dihydroquinoxalin-2-yl)methyl)piperazin-1-yl)benzonitrile in good yield (104 mg, 67%).

Final tetrazole reaction was performed, by mixing the nitrile (100 mg, 0.24 mmol), NaN₃ (125 mg, 1.92 mmol) and Bu₃SnCl (0.48 mL, 1.92 mmol) in toluene (15 mL) at 140° C. for 15 h in a sealed tube. Aq. work-up, as described in method U, followed by column chromatography (SiO₂, 7-8% MeOH in CH₂Cl₂) and final trituration using diethyl ether, afforded the desired tetrazole as a pale-yellow solid (12 mg, 11%).

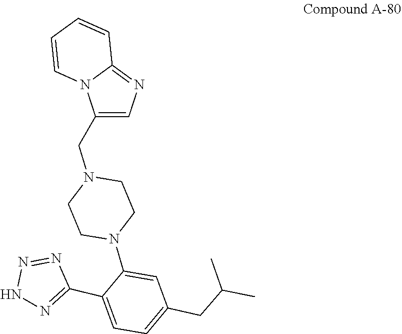 [SMILES: CC(C)Cc1ccc(-c2nn[nH]n2)c(N2CCN(Cc3cnc4ccccn34)CC2)c1]

Prepared through method W from a solution of intermediate 1 (200 mg, 0.71 mmol) in MeOH (20 mL) at 0° C., to which imidazo[1,2-a]pyridine-3-carbaldehyde (110 mg, 0.75 mmol) and a catalytic amount of acetic acid were added. The resulting reaction mixture was stirred at room temperature for 2 h, followed by the addition of sodium cyanoborohydride (133 mg, 2.14 mmol). Upon completion of the addition, the reaction was continued at room temperature for 12 h. Aq. work-up with EtOAc and column chromatography (3-4% MeOH in CH₂Cl₂) yielded 2-(4-(imidazo[1,2-a]pyridin-3-ylmethyl)piperazin-1-yl)-4-isobutylbenzonitrile in moderate yield (123 mg, 46%).

Final tetrazole reaction was performed, by mixing the nitrile (120 mg, 0.32 mmol), NaN₃ (167 mg, 2.57 mmol) and Bu₃SnCl (0.64 mL, 2.57 mmol) in toluene (8 mL) at 140° C. for 20 h in a sealed tube. Aq. work-up, as described in method W, followed by column chromatography (SiO₂, 6-8% MeOH in CH₂Cl₂) and final trituration using diethyl ether, afforded the desired tetrazole as an off-white solid (14 mg, 10%).

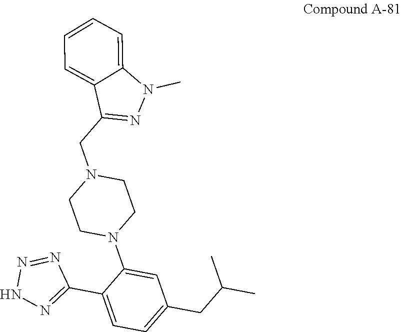 [SMILES: CC(C)Cc1ccc(-c2nn[nH]n2)c(N2CCN(Cc3nn(C)c4ccccc34)CC2)c1]

Prepared through method W from a solution of intermediate 1 (300 mg, 1.07 mmol) in MeOH (30 mL) at 0° C., to which 1-methylindazole-3-carbaldehyde (180 mg, 1.13 mmol) and a catalytic amount of acetic acid were added. The resulting reaction mixture was stirred at room temperature for 3 h, followed by addition of sodium cyanoborohydride (202 mg, 3.21 mmol). Upon completion of the addition, the reaction was continued at room temperature for 12 h. Aq. work-up with EtOAc and column chromatography (34% MeOH in CH₂Cl₂) afforded 4-isobutyl-2-(4-((1-methyl-1H-indazol-3-yl)methyl)piperazin-1-yl)benzonitrile in moderate yield (208 mg, 50%).

Final tetrazole reaction was performed, by mixing the nitrile (200 mg, 0.52 mmol), NaN₃ (268 mg, 4.13 mmol) and Bu₃SnCl (1.03 mL, 4.13 mmol) in toluene (10 mL) at 140 15° C. for 24 h in a sealed tube. Aq. work-up, as described in method W, followed by column chromatography (SiO₂, 6-8% MeOH in CH₂Cl₂) and final trituration using diethyl ether, afforded the envisaged final compound as an off-white solid (31 mg, 14%).

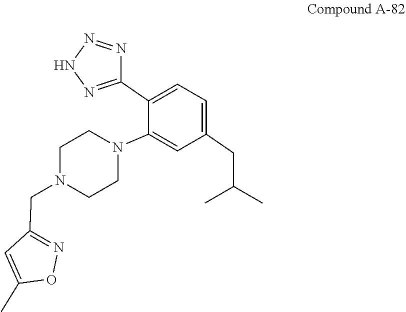 [SMILES: Cc1cc(CN2CCN(c3cc(CC(C)C)ccc3-c3nn[nH]n3)CC2)no1]

Prepared through method U from a solution of intermediate 1 (400 mg, 1.43 mmol) in DMF (20 mL), to which Et₃N (0.79 mL, 5.71 mmol) and 3-(chloromethyl)-5-methyl-isoxazole (226 mg, 1.71 mmol) were added. The resulting reaction mixture was stirred at 80° C. for 8 h. Aq. work-up with EtOAc and column chromatography (2-3% MeOH in CH₂Cl₂) yielded 4-isobutyl-2-(4-((5-methylisoxazol-3-yl)methyl)piperazin-1-yl)benzonitrile in moderate yield (282 mg, 58%).

Final tetrazole reaction was performed, by mixing the nitrile (280 mg, 0.83 mmol), NaN₃ (430 mg, 6.62 mmol) and Bu₃SnCl (1.65 mL, 6.62 mmol) in toluene (15 mL) at 140° C. for 15 h in a sealed tube. Aq. work-up, as described in method U, followed by column chromatography (SiO₂, 6-8% MeOH in CH₂Cl₂) and final trituration using diethyl ether, afforded 3-[[4-[5-isobutyl-2-(2H-tetrazol-5-yl)phenyl]piperazin-1-yl]methyl]-5-methyl-isoxazole as an off-white solid (29 mg, 9%).

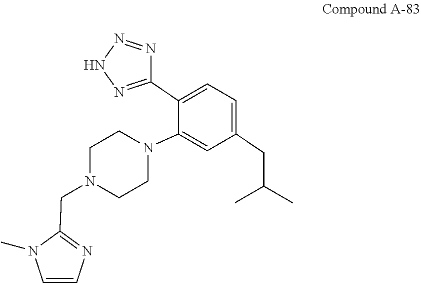 [SMILES: CC(C)Cc1ccc(-c2nn[nH]n2)c(N2CCN(Cc3nccn3C)CC2)c1]

Prepared through method U from a solution of intermediate 1 (300 mg, 1.07 mmol) in DMF (15 mL), to which Et₃N (0.59 mL, 4.29 mmol) and 2-(chloromethyl)-1-methyl-imidazole (168 mg, 1.29 mmol) were added. The resulting reaction mixture was stirred at 80° C. for 6 h. Aq. work-up with EtOAc and column chromatography (2-3% MeOH in CH₂Cl₂) afforded 4-isobutyl-2-(4-((1-methyl-1H-imidazol-2-yl)methyl)piperazin-1-yl)benzonitrile in good yield (265 mg, 73%).

Final tetrazole reaction was performed, by mixing the nitrile (260 mg, 0.77 mmol), NaN₃ (400 mg, 6.16 mmol) and Bu₃SnCl (1.54 mL, 6.16 mmol) in toluene (15 mL) at 140° C. for 12 h in a sealed tube. Aq. work-up, as described in method U, followed by column chromatography (SiO₂, 5-6% MeOH in CH₂Cl₂) and final trituration using diethyl ether, afforded the targeted tetrazole as an off-white solid (9 mg, 3%).

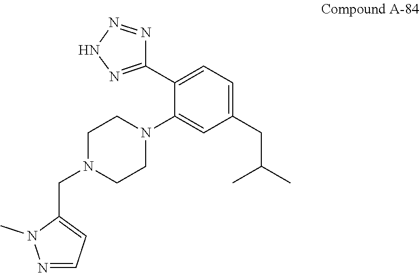 [SMILES: CC(C)Cc1ccc(-c2nn[nH]n2)c(N2CCN(Cc3ccnn3C)CC2)c1]

Prepared through method U from a solution of intermediate 1 (100 mg, 0.36 mmol) in DMF (10 mL), to which Et₃N (0.20 mL, 1.43 mmol) and 5-(chloromethyl)-1-methyl-pyrazole (56 mg, 0.43 mmol) were added. The resulting reaction mixture was stirred at 80° C. for 6 h. Aq. work-up with EtOAc and column chromatography (2-3% MeOH in CH₂Cl₂) afforded 4-isobutyl-2-(4-((1-methyl-1H-pyrazol-5-yl)methyl)piperazin-1-yl)benzonitrile in excellent yield (106 mg, 88%).

Final tetrazole reaction was performed, by mixing the nitrile (100 mg, 0.30 mmol), NaN₃ (154 mg, 2.4 mmol) and Bu₃SnCl (0.59 mL, 2.4 mmol) in toluene (5 mL) at 140° C. for 12 h in a sealed tube. Aq. work-up, as described in method U, followed by column chromatography (SiO₂, 5-6% MeOH in CH₂Cl₂) and final trituration using diethyl ether, afforded 1-[5-isobutyl-2-(2H-tetrazol-5-yl)phenyl]-4-[(1-methylpyrazol-5-yl)methyl]piperazine as an off-white solid (12 mg, 11%).

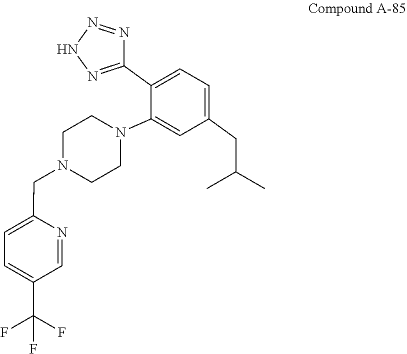 [SMILES: CC(C)Cc1ccc(-c2nn[nH]n2)c(N2CCN(Cc3ccc(C(F)(F)F)cn3)CC2)c1]

Prepared through method U from a solution of intermediate 1 (340 mg, 1.21 mmol) in DMF (25 mL), to which Et₃N (0.67 mL, 4.86 mmol) and 2-(chloromethyl)-5-(trifluoromethyl)pyridine (238 mg, 1.46 mmol) were added. The resulting reaction mixture was stirred at 60° C. for 7 h. Aq. work-up with EtOAc and column chromatography (24% MeOH in CH₂Cl₂) yielded 4-isobutyl-2-(4-((5-(trifluoromethyl)pyridin-2-yl)methyl)piperazin-1-yl)benzonitrile in moderate yield (267 mg, 55%).

Final tetrazole reaction was performed, by mixing the nitrile (265 mg, 0.66 mmol), NaN₃ (342 mg, 5.26 mmol) and Bu₃SnCl (1.32 mL, 5.26 mmol) in toluene (12 mL) at 140° C. for 15 h in a sealed tube. Aq. work-up, as described in method U, followed by column chromatography (SiO₂, 6-8% MeOH in CH₂Cl₂) and final trituration using diethyl ether, afforded the desired tetrazole as a pale-yellow solid (27 mg, 9%).

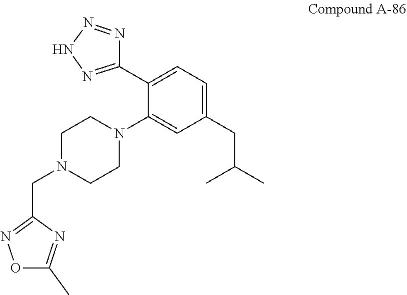 [SMILES: Cc1nc(CN2CCN(c3cc(CC(C)C)ccc3-c3nn[nH]n3)CC2)no1]

Prepared through method U from a solution of intermediate 1 (200 mg, 0.71 mmol) in DMF (15 mL), to which Et₃N (0.40 mL, 2.86 mmol) and 3-(chloromethyl)-5-methyl-1,2,4-oxadiazole (114 mg, 0.86 mmol) were added. The resulting reaction mixture was stirred at 60° C. for 8 h. Aq. work-up with EtOAc and column chromatography (3-5% MeOH in CH₂Cl₂) yielded 4-isobutyl-2-(4-((5-methyl-1,2,4-oxadiazol-3-yl)methyl)piperazin-1-yl)benzonitrile in excellent yield (210 mg, 87%).

Final tetrazole reaction was performed, by mixing the nitrile (200 mg, 0.59 mmol), NaN₃ (306 mg, 4.71 mmol) and Bu₃SnCl (1.43 mL, 4.71 mmol) in toluene (10 mL) at 140° C. for 12 h in a sealed tube. Aq. work-up, as described in method U, followed by column chromatography (SiO₂, 7-8% MeOH in CH₂Cl₂) and final trituration using diethyl ether, afforded 3-[[4-[5-isobutyl-2-(2H-tetrazol-5-yl)phenyl]piperazin-1-yl]methyl]-5-methyl-1,2,4-oxadiazole an off-white solid (18 mg, 8%).

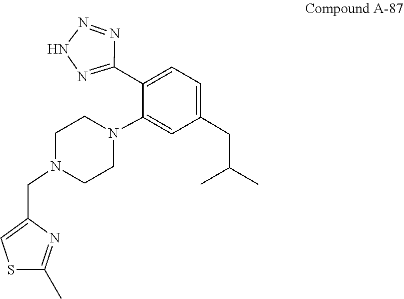 [SMILES: Cc1nc(CN2CCN(c3cc(CC(C)C)ccc3-c3nn[nH]n3)CC2)cs1]

Prepared through method U from a solution of intermediate 1 (300 mg, 1.07 mmol) in DMF (25 mL), to which Et₃N (0.59 mL, 4.29 mmol) and 4-(chloromethyl)-2-methyl-thiazole (190 mg, 1.29 mmol) were added. The resulting reaction mixture was stirred at 60° C. for 8 h. Aq. work-up with EtOAc and column chromatography (3-5% MeOH in CH₂Cl₂) yielded 4-isobutyl-2-(4-((2-methylthiazol-4-yl)methyl)piperazin-1-yl)benzonitrile in good yield (290 mg, 76%).

Final tetrazole reaction was performed, by mixing the nitrile (275 mg, 0.78 mmol), NaN₃ (403 mg, 6.20 mmol) and Bu₃SnCl (1.55 mL, 6.20 mmol) in toluene (10 mL) at 140° C. for 16 h in a sealed tube. Aq. work-up, as described in method U, followed by column chromatography (SiO₂, 7-8% MeOH in CH₂Cl₂) and final trituration using diethyl ether, afforded the desired compound as an off-white solid (17 mg, 6%).

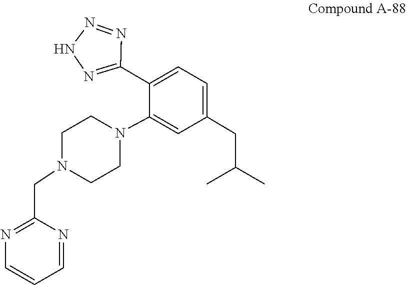 [SMILES: CC(C)Cc1ccc(-c2nn[nH]n2)c(N2CCN(Cc3ncccn3)CC2)c1]

Prepared through method U from a solution of intermediate 1 (300 mg, 1.07 mmol) in DMF (25 mL), to which Et₃N (0.59 mL, 4.29 mmol) and 2-(chloromethyl)pyrimidine (165 mg, 1.29 mmol) were added. The resulting reaction mixture was stirred at 60° C. for 8 h. Aq. work-up with EtOAc and column chromatography (3-5% MeOH in CH₂Cl₂) yielded 4-isobutyl-2-(4-(pyrimidin-2-ylmethyl)piperazin-1-yl)benzonitrile in high yield and purity (308 mg, 86%).

Final tetrazole reaction was performed, by mixing the nitrile (300 mg, 0.89 mmol), NaN₃ (465 mg, 7.15 mmol) and Bu₃SnCl (1.79 mL, 7.15 mmol) in toluene (15 mL) at 140° C. for 18 h in a sealed tube. Aq. work-up, as described in method U, followed by column chromatography (SiO₂, 6-8% MeOH in CH₂Cl₂) and final trituration using diethyl ether, afforded the envisaged tetrazole as a pale-yellow solid (21 mg, 6%).

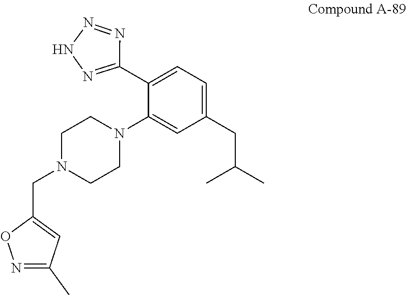 [SMILES: Cc1cc(CN2CCN(c3cc(CC(C)C)ccc3-c3nn[nH]n3)CC2)on1]

Prepared through method U from a solution of intermediate 1 (100 mg, 0.36 mmol) in DMF (6 mL), to which Et₃N (0.20 mL, 1.43 mmol) and 5-(chloromethyl)-3-methyl-isoxazole (47 mg, 0.43 mmol) were added. The resulting reaction mixture was stirred at 80° C. for 7 h. Aq. work-up with EtOAc and column chromatography (3-4% MeOH in CH₂Cl₂) yielded 4-isobutyl-2-(4-((3-methylisoxazol-5-yl)methyl)piperazin-1-yl)benzonitrile in good yield (93 mg, 78%).

Final tetrazole reaction was performed, by mixing the nitrile (90 mg, 0.27 mmol), NaN₃ (138 mg, 2.13 mmol) and Bu₃SnCl (0.53 mL, 2.13 mmol) in toluene (10 mL) at 140° C. for 12 h in a sealed tube. Aq. work-up, as described in method U, followed by column chromatography (SiO₂, 6-7% MeOH in CH₂Cl₂) and final trituration using diethyl ether, afforded the desired tetrazole as an off-white solid (4 mg, 4%).

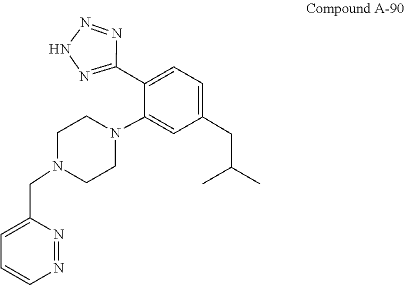 [SMILES: CC(C)Cc1ccc(-c2nn[nH]n2)c(N2CCN(Cc3cccnn3)CC2)c1]

Prepared through method U from pyridazin-3-ylmethanol (100 mg, 0.91 mmol) and SOCl₂ (0.13 mL, 1.82 mmol) to give 3-(chloromethyl)pyridazine as a gummy solid (78 mg, crude). By using intermediate 1 (85 mg, 0.30 mmol), Et₃N (0.17 mL, 1.21 mmol) and 3-(chloromethyl)pyridazine (47 mg, 0.36 mmol) in DMF (8 mL) in a nucleophilic substitution reaction the desired compound was obtained after 8 h stirring at r.t. After performing an aq. work-up, the organic residue was purified by silica chromatography (34% MeOH in CH₂Cl₂) to give 4-isobutyl-2-(4-(pyridazin-3-ylmethyl)piperazin-1-yl)benzonitrile in good yield (82 mg, 81%).

Final tetrazole reaction was performed, by mixing the nitrile (80 mg, 0.24 mmol), NaN₃ (124 mg, 1.90 mmol) and Bu₃SnCl (0.48 mL, 1.90 mmol) in toluene (5 mL) at 140° C. for 15 h in a sealed tube. Aq. work-up, as described in method U, followed by column chromatography (SiO₂, 4-6% MeOH in CH₂Cl₂) and final trituration using diethyl ether, afforded 3-[[4-[5-isobutyl-2-(2H-tetrazol-5-yl)phenyl]piperazin-1-yl]methyl]pyridazine as a pale-yellow solid (11 mg, 12%).

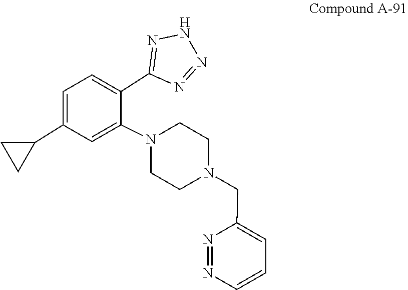 [SMILES: c1cnnc(CN2CCN(c3cc(C4CC4)ccc3-c3nn[nH]n3)CC2)c1]

Prepared through method D from intermediate 37 (0.100 g, 0.44 mmol) and 3-(chloromethyl)pyridazine (0.085 g, 0.66 mmol). The resulting reaction mixture was stirred at 80° C. for 6 h. Aq. work-up with EtOAc and column chromatography (45-50% EtOAc in hexane) yielded 4-cyclopropyl-2-(4-(pyridazin-3-ylmethyl)piperazin-1-yl)benzonitrile in good yield (0.10 g, 71%).

Final tetrazole reaction was performed, by mixing the nitrile (0.10 g, 0.313 mmol), sodium azide (0.163 g, 2.505 mmol) and Bu₃SnCl (0.815 g, 2.505 mmol) in toluene (10 mL) at 140° C. for 15 h in a sealed tube. Aq. work-up, as described in method D, followed by column chromatography (SiO₂, 5-8% MeOH in CH₂Cl₂) and final trituration using diethyl ether, afforded 3-[[4-[5-cyclopropyl-2-(2H-tetrazol-5-yl)phenyl]piperazin-1-yl]methyl]pyridazine as an off-white solid (9 mg, 9%).

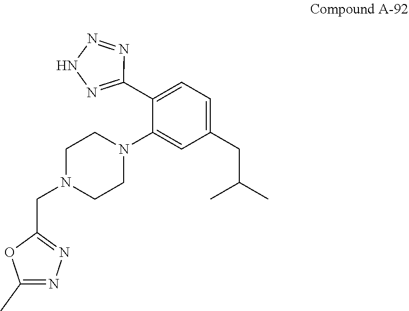 [SMILES: Cc1nnc(CN2CCN(c3cc(CC(C)C)ccc3-c3nn[nH]n3)CC2)o1]

Prepared through method U from a solution of intermediate 1 (380 mg, 1.36 mmol) in DMF (10 mL), to which Et₃N (0.75 mL, 5.43 mmol) and 2-(chloromethyl)-5-methyl-1,3,4-oxadiazole (216 mg, 1.63 mmol) were added. The resulting reaction mixture was stirred at 80° C. for 6 h. Aq. work-up with EtOAc and column chromatography (3-4% MeOH in CH₂Cl₂) yielded 4-isobutyl-2-(4-((5-methyl-1,3,4-oxadiazol-2-yl)methyl)piperazin-1-yl)benzonitrile in moderate yield (213 mg, 46%).

Final tetrazole reaction was performed, by mixing the nitrile (210 mg, 0.62 mmol), NaN₃ (322 mg, 4.95 mmol) and Bu₃SnCl (1.24 mL, 4.95 mmol) in toluene (10 mL) at 140° C. for 16 h in a sealed tube. Aq. work-up, as described in method U, followed by column chromatography (SiO₂, 7-8% MeOH in CH₂Cl₂) and final trituration using diethyl ether, afforded the envisaged tetrazole as an off-white solid (19 mg, 6%).

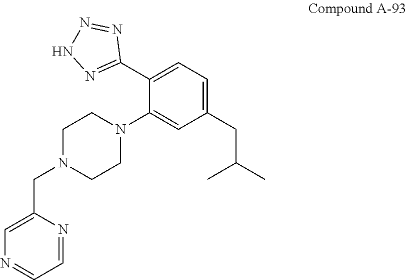 [SMILES: CC(C)Cc1ccc(-c2nn[nH]n2)c(N2CCN(Cc3cnccn3)CC2)c1]

Prepared through method U from a solution of intermediate 1 (170 mg, 0.60 mmol) in DMF (15 mL), to which Et₃N (0.34 mL, 2.43 mmol) and 2-(chloromethyl)pyrazine (93 mg, 0.73 mmol) were added. The resulting reaction mixture was stirred at 60° C. for 8 h. Aq. work-up with EtOAc and column chromatography (3-4% MeOH in CH₂Cl₂) yielded 4-isobutyl-2-(4-(pyrazin-2-ylmethyl)piperazin-1-yl)benzonitrile in good yield (143 mg, 70%).

Final tetrazole reaction was performed, by mixing the nitrile (140 mg, 0.42 mmol), NaN₃ (217 mg, 3.33 mmol) and Bu₃SnCl (0.83 mL, 3.33 mmol) in toluene (15 mL) at 140° C. for 16 h in a sealed tube. Aq. work-up, as described in method U, followed by column chromatography (SiO₂, 6-7% MeOH in CH₂Cl₂) and final trituration using diethyl ether, afforded the envisaged tetrazole as a pale-yellow solid (16 mg, 10%).

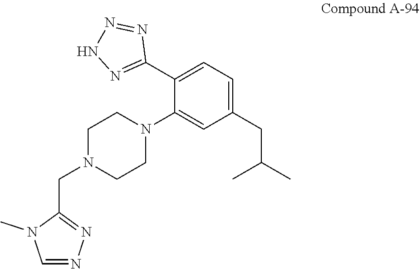 [SMILES: CC(C)Cc1ccc(-c2nn[nH]n2)c(N2CCN(Cc3nncn3C)CC2)c1]

Prepared through method U from a solution of intermediate 1 (165 mg, 0.59 mmol) in DMF (10 mL), to which Et₃N (0.33 mL, 2.36 mmol) and 3-(chloromethyl)-4-methyl-1,2,4-triazole (93 mg, 0.71 mmol) were added. The resulting reaction mixture was stirred at 80° C. for 6 h. Aq. work-up with EtOAc and column chromatography (3-5% MeOH in CH₂Cl₂) yielded 4-isobutyl-2-(4-((4-methyl-4H-1,2,4-triazol-3-yl)methyl)piperazin-1-yl)benzonitrile in good yield (152 mg, 76%).

Final tetrazole reaction was performed, by mixing the nitrile (150 mg, 0.44 mmol), NaN₃ (230 mg, 3.55 mmol) and Bu₃SnCl (0.89 mL, 3.55 mmol) in toluene (6 mL) at 140° C. for 14 h in a sealed tube. Aq. work-up, as described in method U, followed by column chromatography (SiO₂, 5-7% MeOH in CH₂Cl₂) and final trituration using diethyl ether, afforded 1-[5-isobutyl-2-(2H-tetrazol-5-yl)phenyl]-4-[(4-methyl-1,2,4-triazol-3-yl)methyl]piperazine as an off-white solid (12 mg, 7%).

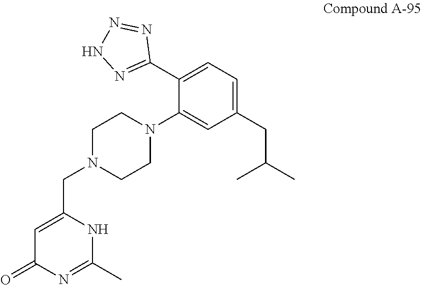 [SMILES: Cc1nc(=O)cc(CN2CCN(c3cc(CC(C)C)ccc3-c3nn[nH]n3)CC2)[nH]1]

Prepared through method U from a solution of intermediate 1 (200 mg, 0.71 mmol) in DMF (15 mL), to which Et₃N (0.40 mL, 2.86 mmol) and 6-(chloromethyl)-2-methyl-1H-pyrimidin-4-one (136 mg, 0.86 mmol) were added. The resulting reaction mixture was stirred at 80° C. for 7 h. Aq. work-up with EtOAc and column chromatography (3-5% MeOH in CH₂Cl₂) afforded 4-isobutyl-2-(4-((2-methyl-6-oxo-3,6-dihydropyrimidin-4-yl)methyl)piperazin-1-yl)benzonitrile in good yield (183 mg, 70%).

Final tetrazole reaction was performed, by mixing the nitrile (180 mg, 0.49 mmol), NaN₃ (256 mg, 3.94 mmol) and Bu₃SnCl (1.0 mL, 3.94 mmol) in toluene (8 mL) at 140° C. for 15 h in a sealed tube. Aq. work-up, as described in method U, followed by column chromatography (SiO₂, 6-8% MeOH in CH₂Cl₂) and final trituration using diethyl ether, delivered 6-[[4-[5-isobutyl-2-(2H-tetrazol-5-yl)phenyl]piperazin-1-yl]methyl]-2-methyl-1H-pyrimidin-4-one as an off-white solid (17 mg, 8%).

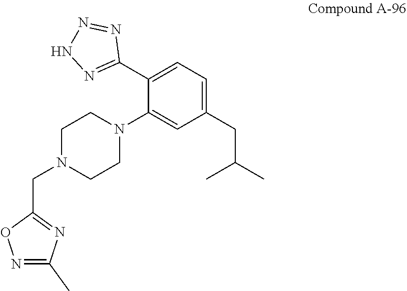 [SMILES: Cc1noc(CN2CCN(c3cc(CC(C)C)ccc3-c3nn[nH]n3)CC2)n1]

Prepared through method U from a solution of intermediate 1 (140 mg, 0.50 mmol) in DMF (6 mL), to which Et₃N (0.28 mL, 2.00 mmol) and 5-(chloromethyl)-3-methyl-1,2,4-oxadiazole (79 mg, 0.60 mmol) were added. The resulting reaction mixture was stirred at 60° C. for 7 h. Aq. work-up with EtOAc and column chromatography (3-4% MeOH in CH₂Cl₂) afforded 4-isobutyl-2-(4-((3-methyl-1,2,4-oxadiazol-5-yl)methyl)piperazin-1-yl)benzonitrile in moderate yield (103 mg, 61%).

Final tetrazole reaction was performed, by mixing the nitrile (100 mg, 0.29 mmol), NaN₃ (153 mg, 2.36 mmol) and Bu₃SnCl (0.59 mL, 2.36 mmol) in toluene (10 mL) at 140° C. for 16 h in a sealed tube. Aq. work-up, as described in method U, followed by column chromatography (SiO₂, 7-8% MeOH in CH₂Cl₂) and final trituration using diethyl ether, yielded the desired final compound as an off-white solid (15 mg, 13%).

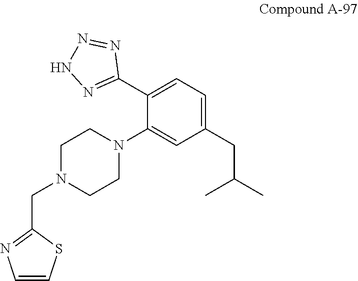 [SMILES: CC(C)Cc1ccc(-c2nn[nH]n2)c(N2CCN(Cc3nccs3)CC2)c1]

Prepared through method U from a solution of intermediate 1 (250 mg, 0.89 mmol) in DMF (15 mL), to which Et₃N (0.50 mL, 3.57 mmol) and 2-(chloromethyl)-thiazole (143 mg, 1.07 mmol) were added. The resulting reaction mixture was stirred at 60° C. for 10 h. Aq. work-up with EtOAc and column chromatography (3-4% MeOH in CH₂Cl₂) afforded 4-isobutyl-2-(4-(thiazol-2-ylmethyl)piperazin-1-yl)benzonitrile in moderate yield (156 mg, 51%).

Final tetrazole reaction was performed, by mixing the nitrile (150 mg, 0.44 mmol), NaN₃ (229 mg, 3.52 mmol) and Bu₃SnCl (0.88 mL, 3.52 mmol) in toluene (15 mL) at 140° C. for 18 h in a sealed tube. Aq. work-up, as described in method U, followed by column chromatography (SiO₂, 7-8% MeOH in CH₂Cl₂) and final trituration using diethyl ether, yielded 2-[[4-[5-isobutyl-2-(2H-tetrazol-5-yl)phenyl]piperazin-1-yl]methyl]thiazole as an off-white solid (19 mg, 11%).

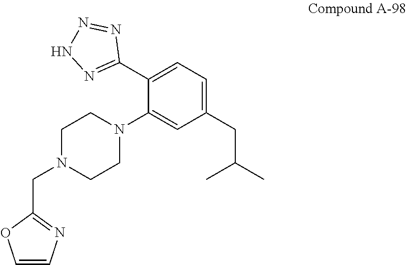 [SMILES: CC(C)Cc1ccc(-c2nn[nH]n2)c(N2CCN(Cc3ncco3)CC2)c1]

Prepared through method U from a solution of intermediate 1 (155 mg, 0.54 mmol) in DMF (8 mL), to which Et₃N (0.30 mL, 2.14 mmol) and 2-(chloromethyl)oxazole (78 mg, 0.66 mmol) were added. The resulting reaction mixture was stirred at 80° C. for 8 h. Aq. work-up with EtOAc and column chromatography (3-4% MeOH in CH₂Cl₂) afforded 4-isobutyl-2-(4-(oxazol-2-ylmethyl)piperazin-1-yl)benzonitrile in moderate yield (106 mg, 57%).

Final tetrazole reaction was performed, by mixing the nitrile (100 mg, 0.31 mmol), NaN₃ (160 mg, 2.47 mmol) and Bu₃SnCl (0.62 mL, 2.47 mmol) in toluene (5 mL) at 140° C. for 15 h in a sealed tube. Aq. work-up, as described in method U, followed by column chromatography (SiO₂, 5-6% MeOH in CH₂Cl₂) and final trituration using diethyl ether, yielded the envisaged tetrazole as an off-white solid (11 mg, 10%).

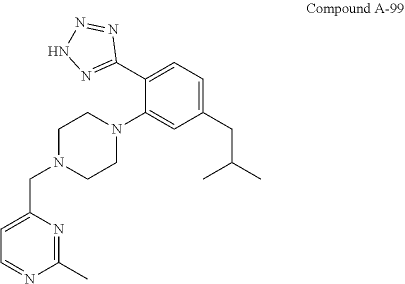 [SMILES: Cc1nccc(CN2CCN(c3cc(CC(C)C)ccc3-c3nn[nH]n3)CC2)n1]

Prepared through method U from a solution of intermediate 1 (250 mg, 0.89 mmol) in DMF (15 mL), to which Et₃N (0.50 mL, 3.57 mmol) and 4-(chloromethyl)-2-methyl-pyrimidine (153 mg, 1.01 mmol) were added. The resulting reaction mixture was stirred at 80° C. for 6 h. Aq. work-up with EtOAc and column chromatography (3-4% MeOH in CH₂Cl₂) afforded 4-isobutyl-2-(4-((2-methylpyrimidin-4-yl)methyl)piperazin-1-yl)benzonitrile in moderate yield (134 mg, 43%).

Final tetrazole reaction was performed, by mixing the nitrile (130 mg, 0.37 mmol), NaN₃ (193 mg, 2.98 mmol) and Bu₃SnCl (0.74 mL, 2.98 mmol) in toluene (8 mL) at 140° C. for 15 h in a sealed tube. Aq. work-up, as described in method U, followed by column chromatography (SiO₂, 6-8% MeOH in CH₂Cl₂) and final trituration using diethyl ether, delivered the desired final compound as an off-white solid (13 mg, 9%).

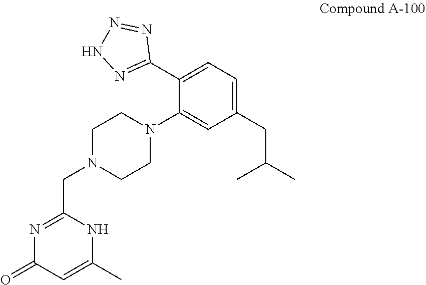 [SMILES: Cc1cc(=O)nc(CN2CCN(c3cc(CC(C)C)ccc3-c3nn[nH]n3)CC2)[nH]1]

Prepared through method U from a solution of intermediate 1 (260 mg, 0.93 mmol) in DMF (15 mL), to which Et₃N (0.51 mL, 3.71 mmol) and 2-(chloromethyl)-6-methyl-1H-pyrimidin-4-one (177 mg, 1.11 mmol) were added. The resulting reaction mixture was stirred at 80° C. for 8 h. Aq. work-up with EtOAc and column chromatography (4-5% MeOH in CH₂Cl₂) afforded 4-isobutyl-2-(4-((6-methyl-4-oxo-1,4-dihydropyrimidin-2-yl)methyl)piperazin-1-yl)benzonitrile in good yield (216 mg, 64%).

Final tetrazole reaction was performed, by mixing the nitrile (210 mg, 0.57 mmol), NaN₃ (300 mg, 4.60 mmol) and Bu₃SnCl (1.15 mL, 4.60 mmol) in toluene (10 mL) at 140° C. for 18 h in a sealed tube. Aq. work-up, as described in method U, followed by column chromatography (SiO₂, 6-8% MeOH in CH₂Cl₂) and final trituration using diethyl ether, delivered 2-[[4-[5-isobutyl-2-(2H-tetrazol-5-yl)phenyl]piperazin-1-yl]methyl]-6-methyl-1H-pyrimidin-4-one as an off-white solid (36 mg, 15%).

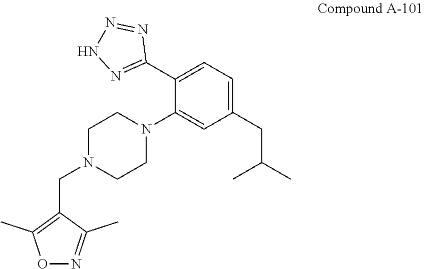 [SMILES: Cc1noc(C)c1CN1CCN(c2cc(CC(C)C)ccc2-c2nn[nH]n2)CC1]

Prepared through method U from a solution of intermediate 1 (400 mg, 1.43 mmol) in DMF (25 mL), to which Et₃N (0.79 mL, 5.71 mmol) and 4-(chloromethyl)-3,5-dimethyl-isoxazole (250 mg, 1.72 mmol) were added. The resulting reaction mixture was stirred at 60° C. for 7 h. Aq. work-up with EtOAc and column chromatography (3-4% MeOH in CH₂Cl₂) afforded 2-(4-((3,5-dimethylisoxazol-4-yl)methyl)piperazin-1-yl)-4-isobutylbenzonitrile in modest yield (286 mg, 57%).

Final tetrazole reaction was performed, by mixing the nitrile (280 mg, 0.79 mmol), NaN₃ (413 mg, 6.35 mmol) and Bu₃SnCl (1.59 mL, 6.35 mmol) in toluene (15 mL) at 140° C. for 16 h in a sealed tube. Aq. work-up, as described in method U, followed by column chromatography (SiO₂, 6-7% MeOH in CH₂Cl₂) and final trituration using diethyl ether, yielded the envisaged compound as an off-white solid (23 mg, 7%).

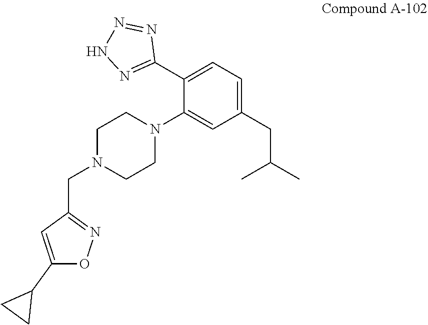 [SMILES: CC(C)Cc1ccc(-c2nn[nH]n2)c(N2CCN(Cc3cc(C4CC4)on3)CC2)c1]

Prepared through method U from a solution of intermediate 1 (200 mg, 0.71 mmol) in DMF (15 mL), to which Et₃N (0.40 mL, 2.86 mmol) and 3-(chloromethyl)-5-cyclopropyl-isoxazole (135 mg, 0.86 mmol) were added. The resulting reaction mixture was stirred at 60° C. for 8 h. Aq. work-up with EtOAc and column chromatography (2-3% MeOH in CH₂Cl₂) yielded 2-(4-((5-cyclopropylisoxazol-3-yl)methyl)piperazin-1-yl)-4-isobutylbenzonitrile in modest yield (126 mg, 48%).

Final tetrazole reaction was performed, by mixing the nitrile (120 mg, 0.33 mmol), NaN₃ (171 mg, 2.63 mmol) and Bu₃SnCl (0.66 mL, 2.63 mmol) in toluene (12 mL) at 140° C. for 14 h in a sealed tube. Aq. work-up, as described in method U, followed by column chromatography (SiO₂, 5-6% MeOH in CH₂Cl₂) and final trituration using diethyl ether, afforded 5-cyclopropyl-3-[[4-[5-isobutyl-2-(2H-tetrazol-5-yl)phenyl]piperazin-1-yl]methyl]isoxazole as an off-white solid (11 mg, 8%).

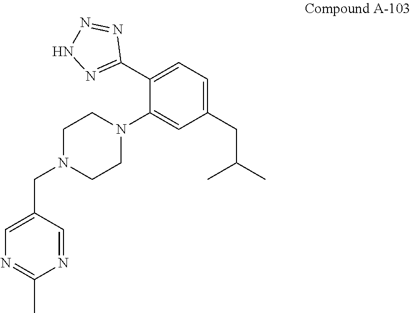 [SMILES: Cc1ncc(CN2CCN(c3cc(CC(C)C)ccc3-c3nn[nH]n3)CC2)cn1]

Prepared through method U from (2-methylpyrimidin-5-yl)methanol (100 mg, 0.80 mmol) and SOCl₂ (0.12 mL, 1.60 mmol) to give 5-(chloromethyl)-2-methylpyrimidine as a gummy liquid (73 mg, crude). To a solution of intermediate 1 (100 mg, 0.36 mmol) in DMF (8 mL) were added Et₃N (0.20 mL, 1.42 mmol) and 5-(chloromethyl)-2-methylpyrimidine (61 mg, 0.43 mmol). The resulting reaction mixture was stirred at r.t. for 8 h. Aq. work-up with EtOAc and column chromatography (3-4% MeOH in CH₂Cl₂) yielded 4-isobutyl-2-(4-((2-methylpyrimidin-5-yl)methyl)piperazin-1-yl)benzonitrile in moderate yield (73 mg, 58%).

Final tetrazole reaction was performed, by mixing the nitrile (70 mg, 0.20 mmol), NaN₃ (104 mg, 1.60 mmol) and Bu₃SnCl (0.40 mL, 1.60 mmol) in toluene (5 mL) at 140° C. for 15 h in a sealed tube. Aq. work-up, as described in method U, followed by column chromatography (SiO₂, 6-7% MeOH in CH₂Cl₂) and final trituration using diethyl ether, afforded the envisaged final compound as an off-white solid (16 mg, 20%).

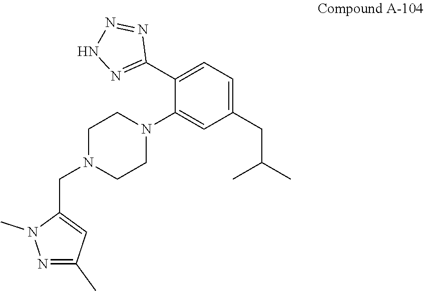 [SMILES: Cc1cc(CN2CCN(c3cc(CC(C)C)ccc3-c3nn[nH]n3)CC2)n(C)n1]

Prepared through method U from a solution of intermediate 1 (300 mg, 1.07 mmol) in DMF (15 mL), to which Et₃N (0.59 mL, 4.29 mmol) and 5-(chloromethyl)-1,3-dimethyl-pyrazole (186 mg, 1.29 mmol) were added. The resulting reaction mixture was stirred at r.t. for 12 h. Aq. work-up with EtOAc and column chromatography (3-4% MeOH in CH₂Cl₂) yielded 2-(4-((1,3-dimethyl-1H-pyrazol-5-yl)methyl)piperazin-1-yl)-4-isobutylbenzonitrile in modest yield (122 mg, 32%).

Final tetrazole reaction was performed, by mixing the nitrile (120 mg, 0.34 mmol), NaN₃ (177 mg, 2.73 mmol) and Bu₃SnCl (0.68 mL, 2.73 mmol) in toluene (6 mL) at 140° C. for 14 h in a sealed tube. Aq. work-up, as described in method U, followed by column chromatography (SiO₂, 5-7% MeOH in CH₂Cl₂) and final trituration using diethyl ether, afforded the desired tetrazole as an off-white solid (20 mg, 15%).

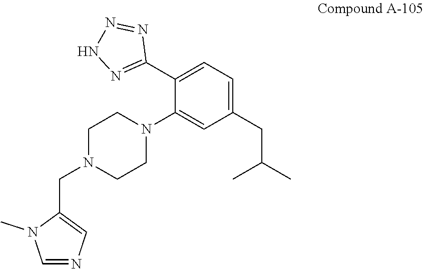 [SMILES: CC(C)Cc1ccc(-c2nn[nH]n2)c(N2CCN(Cc3cncn3C)CC2)c1]

Prepared through method U from (3-methylimidazol-4-yl)methanol (200 mg, 1.78 mmol) and SOCl₂ (0.26 mL, 3.57 mmol) to give 5-(chloromethyl)-1-methyl-imidazole as a gummy solid (155 mg, crude). To a solution of intermediate 1 (150 mg, 0.54 mmol) in DMF (10 mL) were added Et₃N (0.30 mL, 2.14 mmol) and 5-(chloromethyl)-1-methyl-imidazole (84 mg, 0.64 mmol). The resulting reaction mixture was stirred at r.t. for 6 h. Aq. work-up with EtOAc and column chromatography (2-3% MeOH in CH₂Cl₂) yielded 4-isobutyl-2-(4-((1-methyl-1H-imidazol-5-yl)methyl)piperazin-1-yl)benzonitrile in good yield (152 mg, 84%).

Final tetrazole reaction was performed, by mixing the nitrile (150 mg, 0.44 mmol), NaN₃ (231 mg, 3.56 mmol), Bu₃SnCl (0.89 mL, 3.56 mmol) in toluene (10 mL) at 140° C. for 14 h in a sealed tube. Aq. work-up, as described in method U, followed by column chromatography (SiO₂, 4-5% MeOH in CH₂Cl₂) and final trituration using diethyl ether, yielded 1-[5-isobutyl-2-(2H-tetrazol-5-yl)phenyl]-4-[(3-methylimidazol-4-yl)methyl]piperazine as a colorless solid (31 mg, 18%).

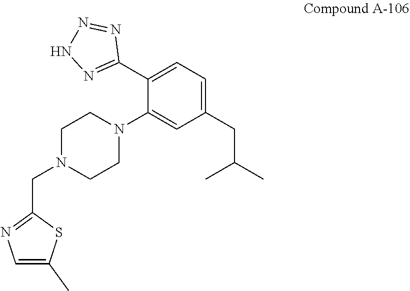 [SMILES: Cc1cnc(CN2CCN(c3cc(CC(C)C)ccc3-c3nn[nH]n3)CC2)s1]

To a stirred solution of intermediate 1 (100 mg, 0.36 mmol) in DMF (10 mL) at 0° C. was added triethylamine (0.20 mL, 1.43 mmol) after which the reaction was stirred at room temperature for 10 minutes. Subsequently, 2-(chloromethyl)-5-methylthiazole (63 mg, 0.43 mmol) was added and the reaction was continued at r.t. for 6 h. After completion of the reaction was confirmed by TLC, the reaction mixture was diluted with cold water and extracted with EtOAc. The combined organic layers were washed with sat. brine, dried over anhydrous sodium sulfate and evaporated under reduced pressure. The crude obtained was purified by column chromatography over silica gel using 3-4% MeOH in CH₂Cl₂ to afford 4-isobutyl-2-(4-((5-methylthiazol-2-yl)methyl)piperazin-1-yl)benzonitrile (93 mg, 74%).

A mixture of 4-isobutyl-2-(4-((5-methylthiazol-2-yl)methyl)piperazin-1-yl)benzonitrile (90 mg, 0.25 mmol), NaN₃ (132 mg, 2.03 mmol) and Bu₃SnCl (0.51 mL, 2.03 mmol) in toluene (10 mL) was stirred at 150° C. for 20 h in a sealed tube. After the completion of the reaction was confirmed by TLC, the reaction mixture was evaporated under reduced pressure and the thus obtained residue was re-dissolved in CH₂Cl₂. The organic layer was washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The crude compound was purified by column chromatography over silica gel, eluting the title compound with 6-7% MeOH in CH₂Cl₂, to afford after an additional trituration with diethyl ether, 2-[[4-[5-isobutyl-2-(2H-tetrazol-5-yl)phenyl]piperazin-1-yl]methyl]-5-methyl-thiazole as an off-white solid (25 mg, 25%).

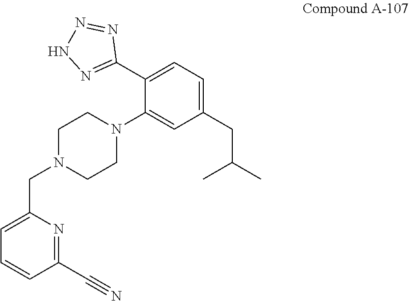 [SMILES: CC(C)Cc1ccc(-c2nn[nH]n2)c(N2CCN(Cc3cccc(C#N)n3)CC2)c1]

Prepared through method V by mixing Boc-protected 4-isobutyl-2-piperazin-1-ylbenzonitrile (1.0 g, 2.91 mmol), NaN₃ (1.51 mg, 23.3 mmol) and Bu₃SnCl (5.82 mL, 23.3 mmol) in toluene (30 mL) at 140° C. for 14 h in a sealed tube. Aq. work-up, as described in method V, followed by column chromatography (SiO₂, 4-6% MeOH in CH₂Cl₂), yielded tert-butyl 4-[5-isobutyl-2-(2H-tetrazol-5-yl)phenyl]piperazine-1-carboxylate as an off-white solid (513 mg, 46%).

Subsequent Boc deprotection through addition of HCl (g) in dioxane (30 mL) to a solution of the tetrazole containing intermediate (0.5 g, 1.29 mmol) in 1,4-dioxane (10 mL) was performed. The resulting mixture was stirred at r.t. for 3 h. Work-up and final trituration with hexane delivered 1-(5-isobutyl-2-(2H-tetrazol-5-yl)phenyl)piperazine hydrochloride which was used as such in the final reaction (363 mg crude).

To a solution of the hydrochloride salt (100 mg, 0.30 mmol) in DMF (10 mL) were added Et₃N (0.17 mL, 1.24 mmol) and 6-(chloromethyl)pyridine-2-carbonitrile (57 mg, 0.37 mmol). The resulting reaction mixture was stirred at 60° C. for 8 h. Aq. work-up with EtOAc as described in method V and column chromatography (5-7% MeOH in CH₂Cl₂) yielded the envisaged compound as an off-white solid (10 mg, 8%).

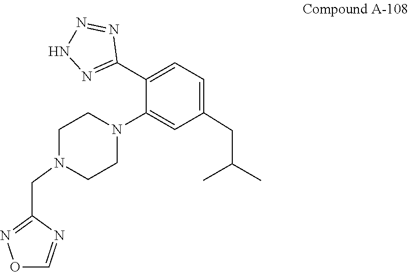 [SMILES: CC(C)Cc1ccc(-c2nn[nH]n2)c(N2CCN(Cc3ncon3)CC2)c1]

Prepared through method V by mixing Boc-protected 4-isobutyl-2-piperazin-1-ylbenzonitrile (1.0 g, 2.91 mmol), NaN₃ (1.51 mg, 23.3 mmol) and Bu₃SnCl (5.82 mL, 23.3 mmol) in toluene (30 mL) at 140° C. for 14 h in a sealed tube. Aq. work-up, as described in method V, followed by column chromatography (SiO₂, 4-6% MeOH in CH₂Cl₂), yielded tert-butyl 4-[5-isobutyl-2-(2H-tetrazol-5-yl)phenyl]piperazine-1-carboxylate as an off-white solid (513 mg, 46%).

Subsequent Boc deprotection through addition of HCl (g) in dioxane (30 mL) to a solution of the tetrazole containing intermediate (0.5 g, 1.29 mmol) in 1,4-dioxane (10 mL) was performed. The resulting mixture was stirred at r.t. for 3 h. Work-up and final trituration with hexane delivered 1-(5-isobutyl-2-(2H-tetrazol-5-yl)phenyl)piperazine hydrochloride which was used as such in the final reaction (363 mg crude).

To a solution of the hydrochloride salt (50 mg, 0.15 mmol) in DMF (5 mL) were added Et₃N (0.086 mL, 0.62 mmol) and 3-(chloromethyl)-1,2,4-oxadiazole (22 mg, 0.18 mmol). The resulting reaction mixture was stirred at 60° C. for 8 h. Aq. work-up with EtOAc and column chromatography (4-6% MeOH in CH₂Cl₂) yielded the targeted tetrazole as an off-white solid (5 mg, 9%).

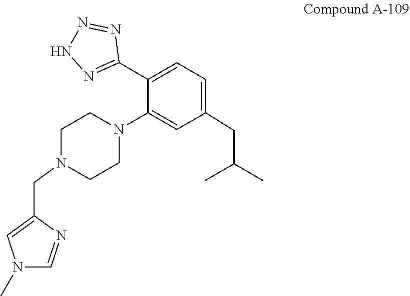 [SMILES: CC(C)Cc1ccc(-c2nn[nH]n2)c(N2CCN(Cc3cn(C)cn3)CC2)c1]

Prepared through method U from (1-methylimidazol-4-yl)methanol (200 mg, 1.78 mmol) and SOCl₂ (0.26 mL, 3.57 mmol) to give 4-(chloromethyl)-1-methyl-imidazole as a gummy solid (155 mg, crude). To a solution of intermediate 1 (150 mg, 0.54 mmol) in DMF (10 mL) were added Et₃N (0.30 mL, 2.14 mmol) and 4-(chloromethyl)-1-methyl-imidazole (84 mg, 0.64 mmol). The resulting reaction mixture was stirred at r.t. for 6 h. Aq. work-up with EtOAc and column chromatography (2-3% MeOH in CH₂Cl₂) yielded 4-isobutyl-2-(4-((1-methyl-1H-imidazol-4-yl)methyl)piperazin-1-yl)benzonitrile as a gummy solid in excellent yield (162 mg, 90%).

Final tetrazole reaction was performed, by mixing the nitrile (160 mg, 0.47 mmol), NaN₃ (246 mg, 3.80 mmol) and Bu₃SnCl (0.95 mL, 3.80 mmol) in toluene (12 mL) at 140° C. for 14 h in a sealed tube. Aq. work-up, as described in method U, followed by column chromatography (SiO₂, 4-5% MeOH in CH₂Cl₂) and final trituration using diethyl ether, delivered the desired tetrazole as an off-white solid (10 mg, 6%).

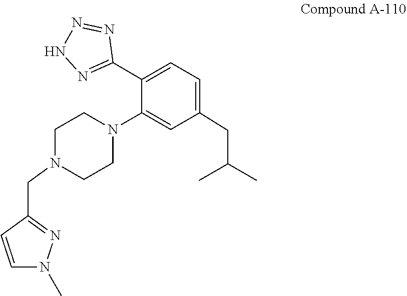 [SMILES: CC(C)Cc1ccc(-c2nn[nH]n2)c(N2CCN(Cc3ccn(C)n3)CC2)c1]

Prepared through method U from (1-methylpyrazol-3-yl)methanol (100 mg, 0.89 mmol) and SOCl₂ (0.13 mL, 1.78 mmol) to give 3-(chloromethyl)-1-methyl-pyrazole as a gummy solid (93 mg, crude). To a solution of intermediate 1 (100 mg, 0.36 mmol) in DMF (10 mL) were added Et₃N (0.20 mL, 1.43 mmol) and 3-(chloromethyl)-1-methyl-pyrazole (56 mg, 0.43 mmol). The resulting reaction mixture was stirred at r.t. for 8 h. Aq. work-up with EtOAc and column chromatography (3-4% MeOH in CH₂Cl₂) afforded 4-isobutyl-2-(4-((1-methyl-1H-pyrazol-3-yl)methyl)piperazin-1-yl)benzonitrile in good yield (93 mg, 78%).

Final tetrazole reaction was performed, by mixing the nitrile (90 mg, 0.27 mmol), NaN₃ (139 mg, 2.13 mmol) and Bu₃SnCl (0.53 mL, 2.13 mmol) in toluene (10 mL) at 140 15° C. for 16 h in a sealed tube. Aq. work-up, as described in method U, followed by column chromatography (SiO₂, 5-6% MeOH in CH₂Cl₂) and final trituration using diethyl ether, delivered the envisaged final compound as an off-white solid (25 mg, 25%).

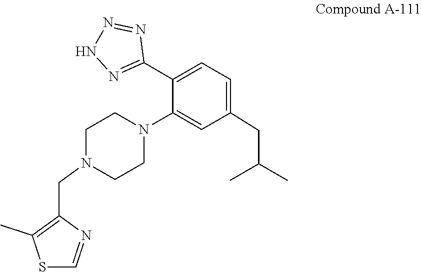 [SMILES: Cc1scnc1CN1CCN(c2cc(CC(C)C)ccc2-c2nn[nH]n2)CC1]

Prepared through method U from (5-methylthiazol-4-yl)methanol (200 mg, 1.55 mmol) and SOCl₂ (0.23 mL, 3.09 mmol) to give 4-(chloromethyl)-5-methyl-thiazole as a gummy solid (162 mg, crude). To a solution of intermediate 1 (150 mg, 0.54 mmol) in DMF (10 mL) were added Et₃N (0.30 mL, 2.14 mmol) and 4-(chloromethyl)-5-methyl-thiazole (95 mg, 0.64 mmol). The resulting reaction mixture was stirred at r.t. for 8 h. Aq. work-up with EtOAc and column chromatography (3-4% MeOH in CH₂Cl₂) yielded 4-isobutyl-2-(4-((5-methylthiazol-4-yl)methyl)piperazin-1-yl)benzonitrile as a gummy solid (164 mg, 86%).

Final tetrazole reaction was performed, by mixing the nitrile (160 mg, 0.45 mmol), NaN₃ (235 mg, 3.61 mmol) and Bu₃SnCl (0.9 mL, 3.61 mmol) in toluene (15 mL) at 140° C. for 20 h in a sealed tube. Aq. work-up, as described in method U, followed by column chromatography (SiO₂, 6-7% MeOH in CH₂Cl₂) and final trituration using diethyl ether, delivered 4-[[4-[5-isobutyl-2-(2H-tetrazol-5-yl)phenyl]piperazin-1-yl]methyl]-5-methyl-thiazole as an off-white solid (10 mg, 6%).

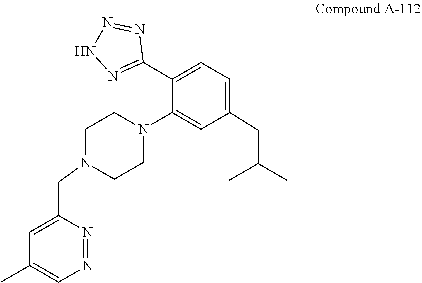 [SMILES: Cc1cnnc(CN2CCN(c3cc(CC(C)C)ccc3-c3nn[nH]n3)CC2)c1]

Prepared through method U from (5-methylpyridazin-3-yl)methanol (100 mg, 0.80 mmol) and SOCl₂ (0.12 mL, 1.60 mmol) to give 3-(chloromethyl)-5-methyl-pyridazine as a gummy solid (103 mg, crude). To a solution of intermediate 1 (100 mg, 0.36 mmol) in DMF (10 mL) were added Et₃N (0.20 mL, 1.43 mmol) and 3-(chloromethyl)-5-methyl-pyridazine (61 mg, 0.43 mmol). The resulting reaction mixture was stirred at r.t. for 10 h. Aq. work-up with EtOAc and column chromatography (3-4% MeOH in CH₂Cl₂) yielded 4-isobutyl-2-(4-((5-methylpyridazin-3-yl)methyl)piperazin-1-yl)benzonitrile in good yield (93 mg, 74%).

Final tetrazole reaction was performed, by mixing the nitrile (90 mg, 0.26 mmol), NaN₃ (134 mg, 2.06 mmol) and Bu₃SnCl (0.52 mL, 2.06 mmol) in toluene (15 mL) at 140° C. for 18 h in a sealed tube. Aq. work-up, as described in method U, followed by column chromatography (SiO₂, 7-8% MeOH in CH₂Cl₂) and final trituration using diethyl ether, afforded 3-[[4-[5-isobutyl-2-(2H-tetrazol-5-yl)phenyl]piperazin-1-yl]methyl]-5-methyl-pyridazine as an off-white solid (8 mg, 8%.

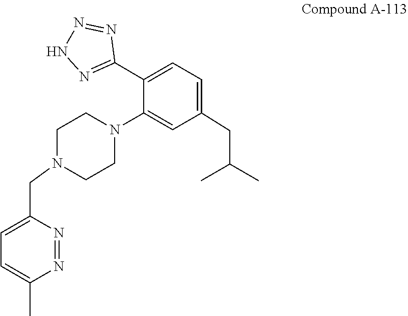 [SMILES: Cc1ccc(CN2CCN(c3cc(CC(C)C)ccc3-c3nn[nH]n3)CC2)nn1]

Prepared through method U from (6-methylpyridazin-3-yl)methanol (200 mg, 1.60 mmol) and SOCl₂ (0.23 mL, 3.20 mmol) to give 3-(chloromethyl)-6-methyl-pyridazine as a gummy solid (143 mg, crude). To a solution of intermediate 1 (150 mg, 0.54 mmol) in DMF (10 mL) were added Et₃N (0.30 mL, 2.14 mmol) and 3-(chloromethyl)-6-methyl-pyridazine (92 mg, 0.64 mmol). The resulting reaction mixture was stirred at r.t. for 8 h. Aq. work-up with EtOAc and column chromatography (3-4% MeOH in CH₂Cl₂) yielded 4-isobutyl-2-(4-((6-methylpyridazin-3-yl)methyl)piperazin-1-yl)benzonitrile in good yield and high purity (153 mg, 82%).

Final tetrazole reaction was performed, by mixing the nitrile (150 mg, 0.43 mmol), NaN₃ (223 mg, 3.43 mmol) and Bu₃SnCl (0.86 mL, 3.43 mmol) in toluene (15 mL) at 140° C. for 16 h in a sealed tube. Aq. work-up, as described in method U, followed by column chromatography (SiO₂, 6-7% MeOH in CH₂Cl₂) and final trituration using diethyl ether, afforded the desired tetrazole as an off-white solid (15 mg, 9%).

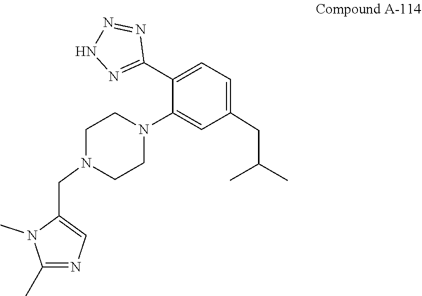 [SMILES: Cc1ncc(CN2CCN(c3cc(CC(C)C)ccc3-c3nn[nH]n3)CC2)n1C]

Prepared through method W from a solution of intermediate 1 (200 mg, 0.71 mmol) in MeOH (20 mL) at 0° C., to which 2,3-dimethylimidazole-4-carbaldehyde (93 mg, 0.75 mmol) and a catalytic amount of acetic acid were added. The resulting reaction mixture was stirred at room temperature for 3 h, followed by addition of sodium cyanoborohydride (135 mg, 2.14 mmol). Upon completion of the addition, the reaction was continued at room temperature for 12 h. Aq. work-up with EtOAc and column chromatography (4-5% MeOH in CH₂Cl₂) afforded 2-(4-((1,2-dimethyl-1H-imidazol-5-yl)methyl)piperazin-1-yl)-4-isobutylbenzonitrile in modest yield (58 mg, 23%).

Final tetrazole reaction was performed, by mixing the nitrile (55 mg, 0.16 mmol), NaN₃ (81 mg, 1.25 mmol) and Bu₃SnCl (0.31 mL, 1.25 mmol) in toluene (10 mL) at 140° C. for 14 h in a sealed tube. Aq. work-up, as described in method W, followed by column chromatography (SiO₂, 6-8% MeOH in CH₂Cl₂) and final trituration using diethyl ether, afforded the envisaged final compound as an off-white solid (6 mg, 10%).

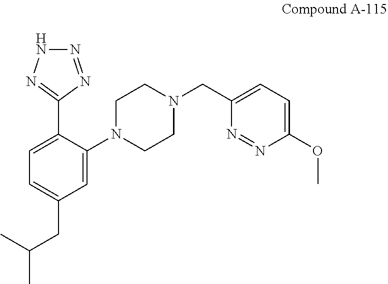 [SMILES: COc1ccc(CN2CCN(c3cc(CC(C)C)ccc3-c3nn[nH]n3)CC2)nn1]

Prepared through method U from (6-methoxypyridazin-3-yl)methanol (100 mg, 0.71 mmol) and SOCl₂ (0.10 mL, 1.42 mmol) to give 3-(chloromethyl)-6-methoxy-pyridazine as a gummy solid (98 mg, crude). To a solution of intermediate 1 (100 mg, 0.36 mmol) in DMF (10 mL) were added Et₃N (0.20 mL, 1.43 mmol) and 3-(chloromethyl)-6-methoxy-pyridazine (68 mg, 0.43 mmol). The resulting reaction mixture was stirred at r.t. for 10 h. Aq. work-up with EtOAc and column chromatography (3-4% MeOH in CH₂Cl₂) afforded 4-isobutyl-2-(4-((6-methoxypyridazin-3-yl)methyl)piperazin-1-yl)benzonitrile in good yield and high purity (96 mg, 73%).

Final tetrazole reaction was performed, by mixing the nitrile (90 mg, 0.25 mmol), NaN₃ (128 mg, 1.97 mmol) and Bu₃SnCl (0.50 mL, 1.97 mmol) in toluene (10 mL) at 140° C. for 20 h in a sealed tube. Aq. work-up, as described in method U, followed by column chromatography (SiO₂, 6-7% MeOH in CH₂Cl₂) and final trituration using diethyl ether, yielded the envisaged final compound as an off-white solid (10 mg, 10%).

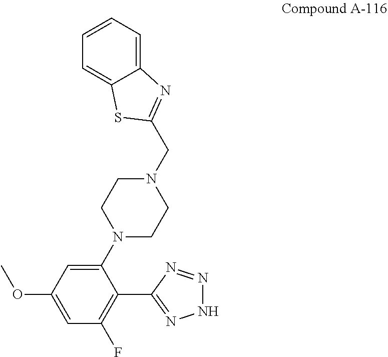 [SMILES: COc1cc(F)c(-c2nn[nH]n2)c(N2CCN(Cc3nc4ccccc4s3)CC2)c1]

Prepared through method C from 2-fluoro-4-methoxy-6-piperazin-1-yl-benzonitrile (intermediate 21, 0.50 g, 2.12 mmol) and 2-(chloromethyl)benzo[d]thiazole (0.582 g, 3.18 mmol). The alkylation reaction was completed after 6 h at 80° C. After performing an aq. work-up, the organic residue was purified by silica chromatography (50-60% EtOAc in hexane) to afford 2-(4-(benzo[d]thiazol-2-ylmethyl)piperazin-1-yl)-6-fluoro-4-methoxybenzonitrile as an off-white solid (0.52 g, 64%).

Final tetrazole reaction was performed, by mixing the nitrile (0.3 g, 0.78 mmol) with sodium azide (0.405 g, 6.24 mmol) and Bu₃SnCl (2.03 g, 6.24 mmol) in toluene (30 mL) at 150° C. for 14 h in a sealed tube. Aq. work-up as described in method C, followed by column chromatography (5-8% MeOH in CH₂Cl₂) and ultimate trituration of the purified compound with diethyl ether yielded the desired compound as an off-white solid (25 mg, 8%).

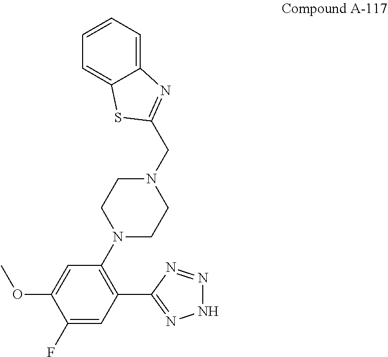 [SMILES: COc1cc(N2CCN(Cc3nc4ccccc4s3)CC2)c(-c2nn[nH]n2)cc1F]

Prepared through method C from 5-fluoro-4-methoxy-2-(piperazin-1-yl)benzonitrile (0.055 g, 0.23 mmol) and 2-(chloromethyl)benzo[d]thiazole (0.063 g, 0.345 mmol). The alkylation reaction was completed after 6 h at 80° C. After performing an aq. work-up and concentration of the organic layers in vacuo, the organic residue was purified by silica chromatography (40-50% EtOAc in hexane) to yield 2-(4-(benzo[d]thiazol-2-ylmethyl)piperazin-1-yl)-5-fluoro-4-methoxybenzonitrile as an off-white solid (0.06 g, 71%).

Final tetrazole reaction was performed, by mixing the nitrile (0.06 g, 0.157 mmol) with sodium azide (0.082 g, 1.25 mmol) and Bu₃SnCl (0.40 g, 1.25 mmol) in toluene (10 mL) at 150° C. for 14 h in a sealed tube. Aq. work-up as described in method C, followed by column chromatography (5-8% MeOH in CH₂Cl₂) and final trituration with diethyl ether, afforded the targeted compound A-117 as an off-white solid (18 mg, 28%).

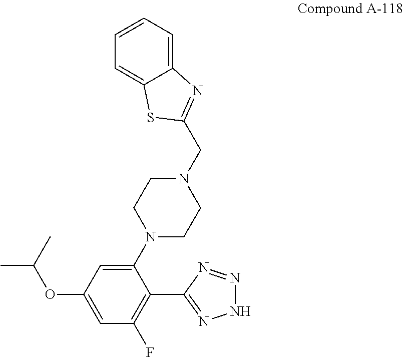 [SMILES: CC(C)Oc1cc(F)c(-c2nn[nH]n2)c(N2CCN(Cc3nc4ccccc4s3)CC2)c1]

Prepared through method C from intermediate 21 (0.44 g, 1.67 mmol) and 2-(chloromethyl)benzo[d]thiazole (0.46 g, 2.50 mmol). The alkylation reaction was completed after 6 h at 80° C. After performing an aq. work-up as described in method C, the organic residue was purified by silica chromatography (30-40% EtOAc in hexane) to afford 2-(4-(benzo[d]thiazol-2-ylmethyl)piperazin-1-yl)-6-fluoro-4-isopropoxybenzonitrile as an off-white solid (0.33 g, 48%).

Final tetrazole reaction was performed, by mixing the nitrile (0.3 g, 0.73 mmol) with sodium azide (0.38 g, 5.80 mmol) and Bu₃SnCl (1.9 g, 5.8 mmol) in toluene (20 mL) at 150° C. for 14 h in a sealed tube. Aq. work-up, followed by column chromatography (5-8% MeOH in CH₂Cl₂) and subsequent trituration of the purified compound with diethyl ether yielded the envisaged tetrazole as an off-white solid (28 mg, 9%).

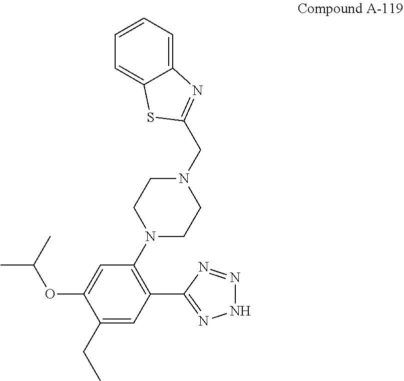 [SMILES: CCc1cc(-c2nn[nH]n2)c(N2CCN(Cc3nc4ccccc4s3)CC2)cc1OC(C)C]

Prepared through method C from 5-ethyl-4-isopropoxy-2-piperazin-1-yl-benzonitrile (intermediate 24, 0.126 g, 0.46 mmol) and 2-(chloromethyl)benzo[d]thiazole (0.13 g, 0.69 mmol). The alkylation reaction was completed after 6 h at 80° C. After performing an aq. work-up as described in method C, the organic residue was purified by silica chromatography (40-50% EtOAc in hexane) to afford 2-(4-(benzo[d]thiazol-2-ylmethyl)piperazin-1-yl)-5-ethyl-4-isopropoxybenzonitrile as a pale-yellow gum (0.14 g, 73%).

Final tetrazole reaction was performed, by mixing the nitrile (0.110 g, 0.261 mmol) with sodium azide (0.136 g, 2.95 mmol) and Bu₃SnCl (0.96 g, 2.95 mmol) in toluene (10 mL) at 150° C. for 14 h in a sealed tube. Aq. work-up, followed by column chromatography (5-8% MeOH in CH₂Cl₂) and ultimate trituration of the purified compound with diethyl ether, yielded the desired substituted tetrazole as a white solid (10 mg, 8%).

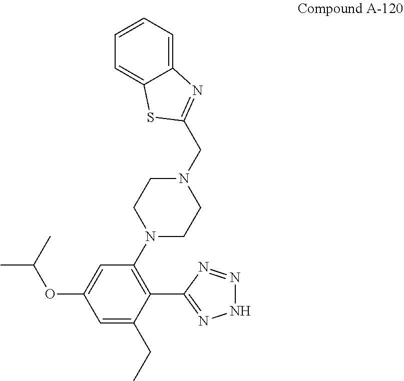 [SMILES: CCc1cc(OC(C)C)cc(N2CCN(Cc3nc4ccccc4s3)CC2)c1-c1nn[nH]n1]

Prepared through method C from 2-ethyl-4-isopropoxy-6-piperazin-1-yl-benzonitrile hydrochloride (intermediate 26, 0.139 g, 0.45 mmol) and 2-(chloromethyl)benzo[d]thiazole (0.123 g, 0.675 mmol). The alkylation reaction was completed after 6 h at 80° C. After performing an aq. work-up as described in method C, the organic residue was purified by silica chromatography (40-50% EtOAc in hexane) to afford 2-(4-(benzo[d]thiazol-2-ylmethyl)piperazin-1-yl)-6-ethyl-4-isopropoxybenzonitrile as an off-white solid (0.17 g, 89%).

Final tetrazole reaction was performed, by mixing the nitrile (0.085 g, 0.20 mmol) with sodium azide (0.105 g, 1.60 mmol) and Bu₃SnCl (0.52 g, 1.60 mmol) in toluene (10 mL) at 150° C. for 14 h in a sealed tube. Aq. work-up as described in method C, followed by column chromatography (5-8% MeOH in CH₂Cl₂) and final trituration using diethyl ether, delivered the envisaged target compound A-120 as an off-white solid (13 mg, 13%).

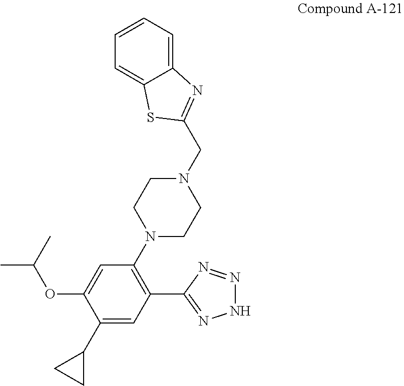 [SMILES: CC(C)Oc1cc(N2CCN(Cc3nc4ccccc4s3)CC2)c(-c2nn[nH]n2)cc1C1CC1]

Prepared through method C from 3-cyclopropyl-4-isopropoxy-6-(piperazin-1-yl)benzonitrile (intermediate 23, 0.131 g, 0.46 mmol) and 2-(chloromethyl)benzo[d]thiazole (0.13 g, 0.69 mmol). The alkylation reaction was completed after 6 h at 80° C. After performing an aq. work-up as described in method C, the organic residue was purified by silica chromatography (40-50% EtOAc in hexane) to afford 2-(4-(benzo[d]thiazol-2-ylmethyl)piperazin-1-yl)-5-cyclopropyl-4-isopropoxybenzonitrile as a pale-yellow gum (0.08 g, 48%).

Final tetrazole reaction was performed, by mixing the nitrile (0.08 g, 0.185 mmol) with sodium azide (0.096 g, 1.48 mmol) and Bu₃SnCl (0.482 g, 1.48 mmol) in toluene (10 mL) at 150° C. for 14 h in a sealed tube. Aq. work-up, followed by column chromatography (5-8% MeOH in CH₂Cl₂) and final trituration with diethyl ether, yielded the desired title compound A-121 as a white solid (0.01 g, 10%).

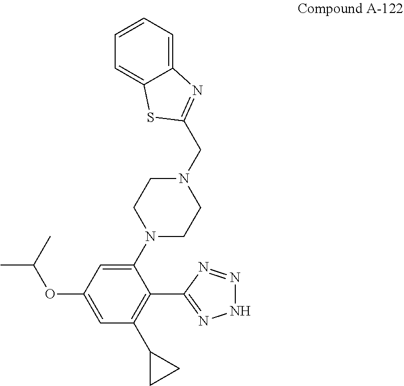 [SMILES: CC(C)Oc1cc(C2CC2)c(-c2nn[nH]n2)c(N2CCN(Cc3nc4ccccc4s3)CC2)c1]

Prepared through method C from 2-cyclopropyl-4-isopropoxy-6-(piperazin-1-yl)-benzonitrile (intermediate 25, 0.20 g, 0.70 mmol) and 2-(chloromethyl)benzo[d]thiazole (0.192 g, 1.05 mmol). The alkylation reaction was completed after 6 h at 80° C. After performing an aq. work-up, the crude organic residue was purified by silica chromatography (40-50% EtOAc in hexane) to afford 2-(4-(benzo[d]thiazol-2-ylmethyl)piperazin-1-yl)-6-cyclopropyl-4-isopropoxybenzonitrile as an off-white solid (0.14 g, 46%).

Final tetrazole reaction was performed, by mixing the nitrile (0.13 g, 0.30 mmol) with sodium azide (0.156 g, 2.40 mmol) and Bu₃SnCl (0.780 g, 2.40 mmol) in toluene (10 mL) at 150° C. for 14 h in a sealed tube. Aq. work-up as described in method C, followed by column chromatography (5-8% MeOH in CH₂Cl₂) and final trituration with diethyl ether, afforded the envisaged substituted tetrazole as an off-white solid (15 mg, 11%).

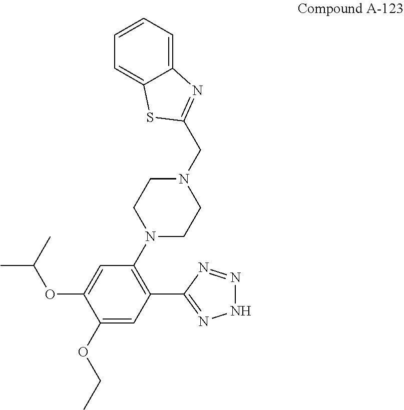 [SMILES: CCOc1cc(-c2nn[nH]n2)c(N2CCN(Cc3nc4ccccc4s3)CC2)cc1OC(C)C]

Prepared through method C from 5-ethoxy-4-isopropoxy-2-(piperazin-1-yl)benzonitrile hydrochloride (intermediate 22, 0.16 g, 0.49 mmol) and 2-(chloromethyl)benzo[d]thiazole (0.135 g, 0.73 mmol). The alkylation reaction was completed after 6 h at 80° C. After performing an aq. work-up, the organic residue was purified by silica chromatography (40-50% EtOAc in hexane) to afford 2-(4-(benzo[d]thiazol-2-ylmethyl)piperazin-1-yl)-5-ethoxy-4-isopropoxybenzonitrile as an off-white solid (0.18 g, 81%). Final tetrazole reaction was performed, by mixing the nitrile (0.18 g, 0.41 mmol) with sodium azide (0.214 g, 3.30 mmol) and Bu₃SnCl (1.07 g, 3.30 mmol) in toluene (10 mL) at 150° C. for 14 h in a sealed tube. Aq. work-up as described in method C, followed by column chromatography (5-8% MeOH in CH₂Cl₂) and subsequent trituration of the purified compound with diethyl ether, yielded the desired substituted tetrazole as a brown solid (0.01 g, 5%).

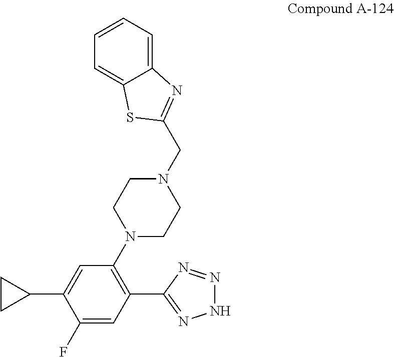 [SMILES: Fc1cc(-c2nn[nH]n2)c(N2CCN(Cc3nc4ccccc4s3)CC2)cc1C1CC1]

Prepared through method C from intermediate 30 (0.233 g, 0.951 mmol) and 2-(chloromethyl)benzo[d]thiazole (0.262 g, 1.43 mmol). The alkylation reaction was completed after 6 h at 80° C. After performing an aq. work-up, the organic residue was purified by silica chromatography (40-45% EtOAc in hexane) to afford 2-(4-(benzo[d]thiazol-2-ylmethyl)piperazin-1-yl)-4-cyclopropyl-5-fluorobenzonitrile as a gummy liquid (0.30 g, 80%).

Final tetrazole reaction was performed, by mixing the nitrile (0.30 g, 0.765 mmol) with sodium azide (0.397 g, 6.12 mmol) and Bu₃SnCl (1.99 g, 6.12 mmol) in toluene (15 mL) at 150° C. for 14 h in a sealed tube. Aq. work-up as described in method C, followed by column chromatography (6-8% MeOH in CH₂Cl₂) and subsequent trituration with diethyl ether, yielded the desired title compound A-124 as an off-white solid (27 mg, 8%).

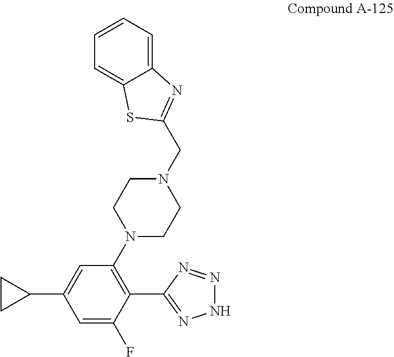 [SMILES: Fc1cc(C2CC2)cc(N2CCN(Cc3nc4ccccc4s3)CC2)c1-c1nn[nH]n1]

Prepared through method C from intermediate 27 (0.102 g, 0.417 mmol) and 2-(chloromethyl)benzo[d]thiazole (0.115 g, 0.626 mmol). The alkylation reaction was completed after 6 h at 80° C. Aq. work-up, followed by silica chromatography (45-50% EtOAc in hexane) afforded 2-(4-(benzo[d]thiazol-2-ylmethyl)piperazin-1-yl)-4-cyclopropyl-6-fluorobenzonitrile as a gummy liquid (0.09 g, 56%).

Final tetrazole reaction was performed, by mixing the nitrile (0.085 g, 0.216 mmol) with sodium azide (0.113 g, 1.734 mmol) and Bu₃SnCl (0.563 g, 1.734 mmol) in toluene (10 mL) at 150° C. for 14 h in a sealed tube. Aq. work-up and purification via column chromatography over silica gel (8-10% MeOH in CH₂Cl₂), followed by final trituration of the purified compound using diethyl ether, yielded the targeted tetrazole as an off-white solid (8 mg, 9%).

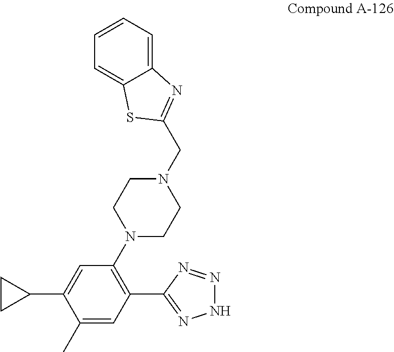 [SMILES: Cc1cc(-c2nn[nH]n2)c(N2CCN(Cc3nc4ccccc4s3)CC2)cc1C1CC1]

Prepared through method C from intermediate 32 (0.15 g, 0.622 mmol) and 2-(chloromethyl)benzo[d]thiazole (0.17 g, 0.933 mmol). The alkylation reaction was completed after 6 h at 80° C. Aq. work-up, followed by silica chromatography (45-50% EtOAc in hexane) afforded 2-(4-(benzo[d]thiazol-2-ylmethyl)piperazin-1-yl)-4-cyclopropyl-5-methylbenzonitrile as an off-white solid (0.21 g, 88%).

Final tetrazole reaction was performed, by mixing the nitrile (0.21 g, 0.54 mmol) with sodium azide (0.176 g, 2.70 mmol) and Bu₃SnCl (0.877 g, 2.70 mmol) in toluene (20 mL) at 150° C. for 14 h in a sealed tube. Aq. work-up, followed by purification via column chromatography over silica gel (6-8% MeOH in CH₂Cl₂) and ultimate trituration of the purified compound with diethyl ether, yielded the envisaged substituted tetrazole as an off-white solid (30 mg, 13%).

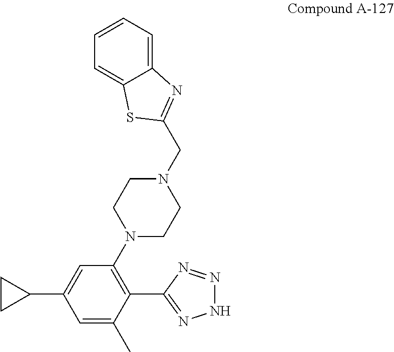 [SMILES: Cc1cc(C2CC2)cc(N2CCN(Cc3nc4ccccc4s3)CC2)c1-c1nn[nH]n1]

Prepared through method C from intermediate 6 (0.11 g, 0.397 mmol) and 2-(chloromethyl)benzo[d]thiazole (0.109 g, 0.595 mmol). The alkylation reaction was completed after 6 h at 80° C. After performing an aq. work-up, the organic residue was purified by silica chromatography (45-50% EtOAc in hexane) to afford 2-(4-(benzo[d]thiazol-2-ylmethyl)piperazin-1-yl)-4-cyclopropyl-6-methylbenzonitrile as a gummy liquid (0.12 g, 79%).

Final tetrazole reaction followed by aq. work-up, column chromatography (6-8% MeOH in CH₂Cl₂) and ultimate trituration of the purified compound with diethyl ether, yielded the desired compound as an off-white solid (10 mg, 8%).

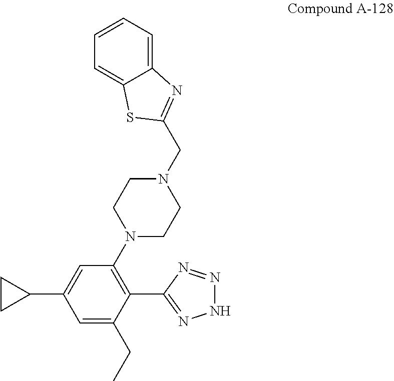 [SMILES: CCc1cc(C2CC2)cc(N2CCN(Cc3nc4ccccc4s3)CC2)c1-c1nn[nH]n1]

Prepared through method C from intermediate 28 (0.19 g, 0.66 mmol) and 2-(chloromethyl)benzo[d]thiazole (0.181 g, 0.99 mmol). The alkylation reaction was completed after 6 h at 80° C. Aq. work-up as described in method C, followed by silica chromatography (45-50% EtOAc in hexane) afforded 2-(4-(benzo[d]thiazol-2-ylmethyl)piperazin-1-yl)-4-cyclopropyl-6-ethylbenzonitrile as an off-white solid (0.18 g, 46%).

Final tetrazole reaction was performed, by mixing the nitrile (0.175 g, 0.434 mmol) with sodium azide (0.226 g, 3.48 mmol) and Bu₃SnCl (1.13 g, 3.48 mmol) in toluene (15 mL) at 150° C. for 14 h in a sealed tube. Aq. work-up, followed by purification via column chromatography over silica gel (5-8% MeOH in CH₂Cl₂) and subsequent trituration using diethyl ether, yielded the targeted substituted tetrazole A-128 as an off-white solid (35 mg, 18%).

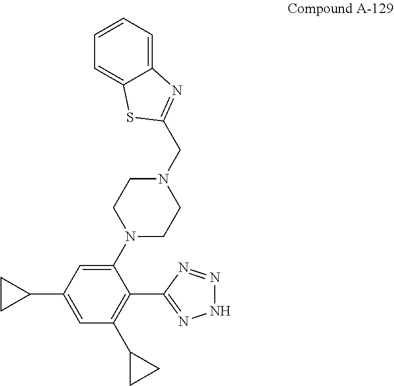 [SMILES: c1ccc2sc(CN3CCN(c4cc(C5CC5)cc(C5CC5)c4-c4nn[nH]n4)CC3)nc2c1]

Prepared through method C from intermediate 7 (0.20 g, 0.66 mmol) and 2-(chloromethyl)benzo[d]thiazole (0.181 g, 0.99 mmol). The alkylation reaction was completed after 6 h at 80° C. After performing an aq. work-up, the organic residue was purified by silica chromatography (45-50% EtOAc in hexane) to afford 2-(4-(benzo[d]thiazol-2-ylmethyl)piperazin-1-yl)-4,6-dicyclopropylbenzonitrile as an off-white solid (0.18 g, 46%).

Final tetrazole reaction followed by aq. work-up, column chromatography (5-8% MeOH in CH₂Cl₂) and ultimate trituration of the purified compound with diethyl ether yielded the desired compound as an off-white solid (35 mg, 18%).

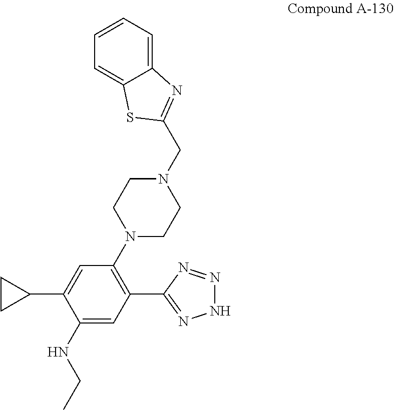 [SMILES: CCNc1cc(-c2nn[nH]n2)c(N2CCN(Cc3nc4ccccc4s3)CC2)cc1C1CC1]

Prepared through method C from intermediate 35 (0.060 g, 0.174 mmol) and 2-(chloromethyl)benzo[d]thiazole (0.048 g, 0.261 mmol). The alkylation reaction was completed after 6 h at 80° C. Aq. work-up, followed by silica chromatography (25-30% EtOAc in hexane) afforded 2-(4-(benzo[d]thiazol-2-ylmethyl)piperazin-1-yl)-4-cyclopropyl-5-(ethylamino)benzonitrile hydrochloride as an off-white solid (0.06 g, 82%).

Final tetrazole reaction was performed, by mixing the nitrile (0.060 g, 0.132 mmol) with sodium azide (0.069 g, 1.057 mmol) and Bu₃SnCl (0.343 g, 1.057 mmol) in toluene (8 mL) at 150° C. for 14 h in a sealed tube. Aq. work-up, followed by purification via column chromatography over silica gel (5-7% MeOH in CH₂Cl₂) and subsequent trituration using diethyl ether yielded the targeted title compound A-130 as an off-white solid (7 mg, 11%).

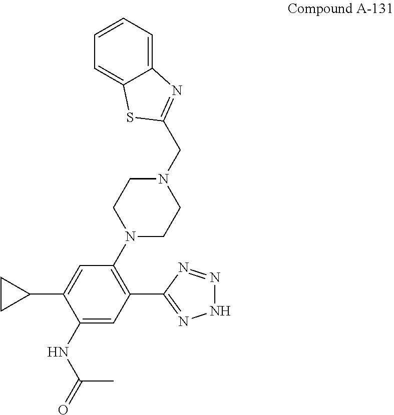 [SMILES: CC(=O)Nc1cc(-c2nn[nH]n2)c(N2CCN(Cc3nc4ccccc4s3)CC2)cc1C1CC1]

Prepared through method C from intermediate 36 (0.120 g, 0.422 mmol) and 2-(chloromethyl)benzo[d]thiazole (0.116 g, 0.633 mmol). The alkylation reaction was completed after 6 h at 80° C. Aq. work-up, followed by silica chromatography (40-45% EtOAc in hexane) afforded N-(4-(4-(benzo[d]thiazol-2-ylmethyl)piperazin-1-yl)-5-cyano-2-cyclopropylphenyl)acetamide as a colorless gum (0.09 g, 49%).

Final tetrazole reaction was performed, by mixing the nitrile (0.080 g, 0.186 mmol) with sodium azide (0.096 g, 1.484 mmol) and Bu₃SnCl (0.482 g, 1.484 mmol) in toluene (10 mL) at 150° C. for 14 h in a sealed tube. Aq. work-up and column chromatography over silica gel (6-8% MeOH in CH₂Cl₂), followed by trituration of the purified compound with diethyl ether yielded the desired substituted tetrazole as an off-white solid (7 mg, 9%).

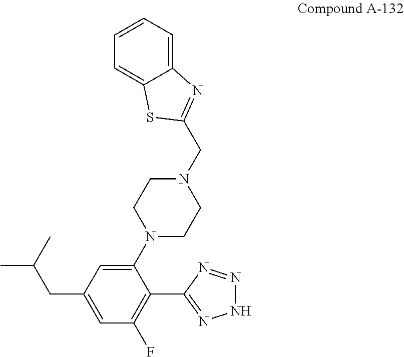 [SMILES: CC(C)Cc1cc(F)c(-c2nn[nH]n2)c(N2CCN(Cc3nc4ccccc4s3)CC2)c1]

Prepared through method C from intermediate 4 (0.12 g, 0.404 mmol) and 2-(chloromethyl)benzo[d]thiazole (0.111 g, 0.606 mmol). The alkylation reaction was completed after 6 h at 80° C. After performing an aq. work-up, the organic residue was purified by silica chromatography (40-50% EtOAc in hexane) to give 2-(4-(benzo[d]thiazol-2-ylmethyl)piperazin-1-yl)-6-fluoro-4-isobutylbenzonitrile as an off-white solid (110 mg, 69%).

Final tetrazole reaction followed by aq. work-up, column chromatography (5-8% MeOH in CH₂Cl₂) and ultimate trituration of the purified compound with diethyl ether, yielded the envisaged compound as an off-white solid (13 mg, 12%).

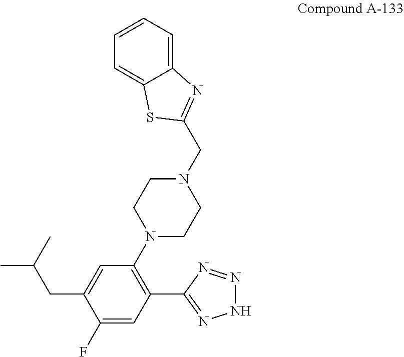 [SMILES: CC(C)Cc1cc(N2CCN(Cc3nc4ccccc4s3)CC2)c(-c2nn[nH]n2)cc1F]

Prepared through method C from intermediate 29 (0.12 g, 0.404 mmol) and 2-(chloromethyl)benzo[d]thiazole (0.111 g, 0.606 mmol). The alkylation reaction was completed after 6 h at 80° C. After performing an aq. work-up as described in method C, the obtained crude residue was purified by silica chromatography (45-50% EtOAc in hexane) to give 2-(4-(benzo[d]thiazol-2-ylmethyl)piperazin-1-yl)-5-fluoro-4-isobutylbenzonitrile as an off-white solid (85 mg, 52%).

Final tetrazole reaction was performed, by mixing the nitrile (0.070 g, 0.172 mmol) with sodium azide (0.090 g, 1.372 mmol) and Bu₃SnCl (0.446 g, 1.372 mmol) in toluene (10 mL) at 150° C. for 14 h in a sealed tube. Aq. work-up as described in method C, followed by column chromatography (5-8% MeOH in CH₂Cl₂) and subsequent trituration of the purified compound with diethyl ether, afforded the desired substituted tetrazole as an off-white solid (0.011 g, 9%).

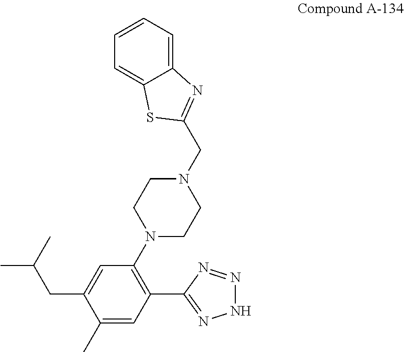 [SMILES: Cc1cc(-c2nn[nH]n2)c(N2CCN(Cc3nc4ccccc4s3)CC2)cc1CC(C)C]

Prepared through method C from intermediate 31 (0.15 g, 0.505 mmol) and 2-(chloromethyl)benzo[d]thiazole (0.14 g, 0.758 mmol). The alkylation reaction was completed after 6 h at 80° C. After performing an aq. work-up, the crude residue was purified by silica chromatography (45-50% EtOAc in hexane) to give 2-(4-(benzo[d]thiazol-2-ylmethyl)piperazin-1-yl)-4-isobutyl-5-methylbenzonitrile as an off-white solid (0.14 g, 70%).

Final tetrazole reaction was performed, by mixing the nitrile (0.13 g, 0.322 mmol) with sodium azide (0.167 g, 2.57 mmol) and Bu₃SnCl (0.835 g, 2.57 mmol) in toluene (10 mL) at 150° C. for 14 h in a sealed tube. After an aq. work-up and subsequent column chromatography (5-8% MeOH in CH₂Cl₂), an ultimate trituration of the purified compound with diethyl ether yielded the envisaged title compound A-134 as an off-white solid (18 mg, 12%).

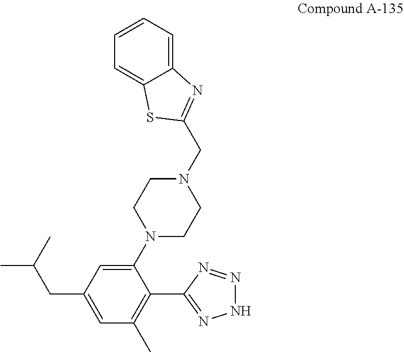 [SMILES: Cc1cc(CC(C)C)cc(N2CCN(Cc3nc4ccccc4s3)CC2)c1-c1nn[nH]n1]

Prepared through method C from intermediate 5 (0.07 g, 0.238 mmol) and 2-(chloromethyl)benzo[d]thiazole (0.065 g, 0.357 mmol). The alkylation reaction was completed after 6 h at 80° C. After performing an aq. work-up, the organic residue was purified by silica chromatography (40-50% EtOAc in hexane) to give 2-(4-(benzo[d]thiazol-2-ylmethyl)piperazin-1-yl)-4-isobutyl-6-methylbenzonitrile as an off-white solid (50 mg, 45%).

Final tetrazole reaction, followed by aq. work-up, column chromatography (5-8% MeOH in CH₂Cl₂) and ultimate trituration of the purified compound with diethyl ether, yielded the desired compound as a pale-yellow solid (6 mg, 13%).

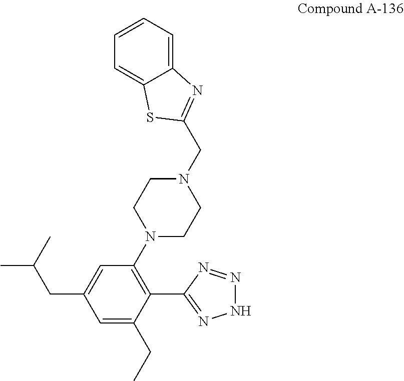 [SMILES: CCc1cc(CC(C)C)cc(N2CCN(Cc3nc4ccccc4s3)CC2)c1-c1nn[nH]n1]

Prepared through method C from intermediate 9 (0.12 g, 0.39 mmol) and 2-(chloromethyl)benzo[d]thiazole (0.107 g, 0.585 mmol). The alkylation reaction was completed after 6 h at 80° C. After performing an aq. work-up, purification via column chromatography over silica gel (40-45% EtOAc in hexane) gave 2-(4-(benzo[d]thiazol-2-ylmethyl)piperazin-1-yl)-6-ethyl-4-isobutylbenzonitrile as an off-white solid (0.12 g, 73%).

Final tetrazole reaction was performed, by mixing the nitrile (0.12 g, 0.29 mmol) with sodium azide (0.149 g, 2.29 mmol) and Bu₃SnCl (0.744 g, 2.29 mmol) in toluene (10 mL) at 150° C. for 14 h in a sealed tube. Aq. work-up as described in method C, followed by column chromatography (5-8% MeOH in CH₂Cl₂) and final trituration of the purified compound using diethyl ether afforded the targeted title compound A-136 as an off-white solid (13 mg, 10%).

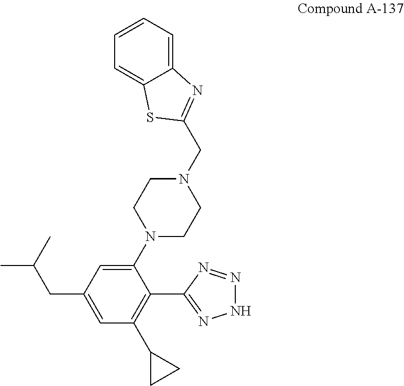 [SMILES: CC(C)Cc1cc(C2CC2)c(-c2nn[nH]n2)c(N2CCN(Cc3nc4ccccc4s3)CC2)c1]

Prepared through method C from intermediate 8 (0.061 g, 0.19 mmol) and 2-(chloromethyl)benzo[d]thiazole (0.052 g, 0.285 mmol). The alkylation reaction was completed after 6 h at 80° C. After performing an aq. work-up, the crude residue was purified by column chromatography over silica gel (50-55% EtOAc in hexane) to give 2-(1-(benzo[d]thiazol-2-ylmethyl)piperidin-4-yl)-6-cyclopropyl-4-isobutylbenzonitrile as an off-white solid (67 mg, 82%).

Final tetrazole reaction was performed, by mixing the nitrile (0.060 g, 0.14 mmol) with sodium azide (0.073 g, 1.11 mmol) and Bu₃SnCl (0.361 g, 1.11 mmol) in toluene (8 mL) at 150° C. for 14 h in a sealed tube. Aq. work-up as described in method C, followed by column chromatography (6-8% MeOH in CH₂Cl₂) and subsequent trituration of the purified compound with diethyl ether, afforded the desired substituted tetrazole as an off-white solid (7 mg, 11%).

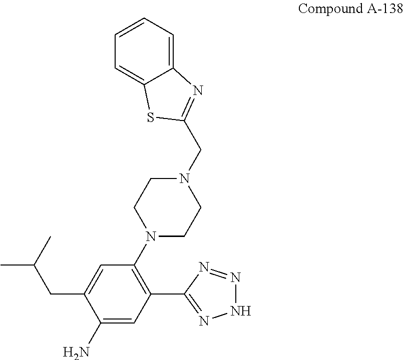 [SMILES: CC(C)Cc1cc(N2CCN(Cc3nc4ccccc4s3)CC2)c(-c2nn[nH]n2)cc1N]

Prepared through method C from intermediate 34 (0.11 g, 0.332 mmol) and 2-(chloromethyl)benzo[d]thiazole (0.09 g, 0.498 mmol). The alkylation reaction was completed after 6 h at 80° C. After performing an aq. work-up as described in method C, the crude organic residue was purified by silica chromatography (50-55% EtOAc in hexane) to afford 5-amino-2-(4-(benzo[d]thiazol-2-ylmethyl)piperazin-1-yl)-4-isobutylbenzonitrile as an off-white solid (0.070 g, 52%).

Final tetrazole reaction was performed, by mixing the nitrile (0.070 g, 0.173 mmol) with sodium azide (0.090 g, 1.38 mmol) and Bu₃SnCl (0.448 g, 1.38 mmol) in toluene (5 mL) at 150° C. for 14 h in a sealed tube. After an aq. work-up and subsequent column chromatography over silica gel (5-8% MeOH in CH₂Cl₂), a trituration of the purified compound using diethyl ether yielded the desired substituted tetrazole as an off-white solid (12 mg, 9%).

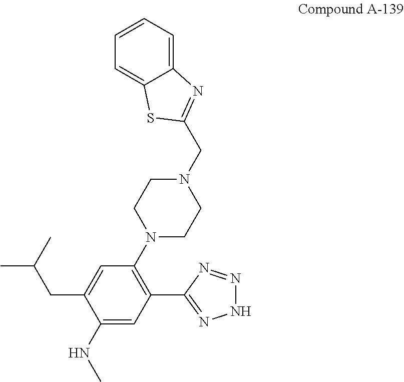 [SMILES: CNc1cc(-c2nn[nH]n2)c(N2CCN(Cc3nc4ccccc4s3)CC2)cc1CC(C)C]

Prepared through method C from 4-isobutyl-5-(methylamino)-2-(piperazin-1-yl)benzonitrile dihydrochloride (intermediate 33, 0.10 g, 0.289 mmol) and 2-(chloromethyl)benzo[d]thiazole (0.08 g, 0.433 mmol). The alkylation reaction was completed after 6 h at 80° C. After performing an aq. work-up as described in method C, the crude organic residue was purified by silica chromatography (45-50% EtOAc in hexane) to afford 2-(4-(benzo[d]thiazol-2-ylmethyl)piperazin-1-yl)-4-isobutyl-5-(methylamino)benzonitrile as an off-white solid (0.080 g, 82%).

Final tetrazole reaction was performed, by mixing the nitrile (0.075 g, 0.179 mmol) with sodium azide (0.093 g, 1.432 mmol) and Bu₃SnCl (0.466 g, 1.432 mmol) in toluene (10 mL) at 150° C. for 14 h in a sealed tube. After an aq. work-up and subsequent column chromatography over silica gel (5-8% MeOH in CH₂Cl₂), a trituration of the purified compound with diethyl ether gave the targeted substituted tetrazole as an off-white solid (11 mg, 13%).

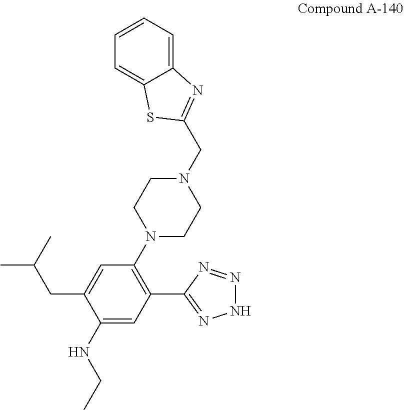 [SMILES: CCNc1cc(-c2nn[nH]n2)c(N2CCN(Cc3nc4ccccc4s3)CC2)cc1CC(C)C]

Prepared through method C from 5-(ethylamino)-4-isobutyl-2-(piperazin-1-yl)benzonitrile dihydrochloride (intermediate 33, 0.17 g, 0.473 mmol) and 2-(chloromethyl)benzo[d]thiazole (0.13 g, 0.710 mmol). The alkylation reaction was completed after 6 h at 80° C. After performing an aq. work-up as described in method C, the obtained crude residue was purified by silica chromatography (45-50% EtOAc in hexane) to afford 2-(4-(benzo[d]thiazol-2-ylmethyl)piperazin-1-yl)-5-(ethylamino)-4-isobutylbenzonitrile as an off-white solid (0.080 g, 36%).

Final tetrazole reaction was performed, by mixing the nitrile (0.075 g, 0.173 mmol) with sodium azide (0.090 g, 1.385 mmol) and Bu₃SnCl (0.450 g, 1.385 mmol) in toluene (10 mL) at 150° C. for 14 h in a sealed tube. After an aq. work-up and subsequent column chromatography over silica gel (5-8% MeOH in CH₂Cl₂), a final trituration using diethyl ether gave the envisaged title compound as an off-white solid (13 mg, 16%).

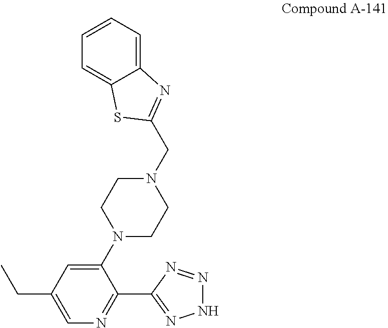 [SMILES: CCc1cnc(-c2nn[nH]n2)c(N2CCN(Cc3nc4ccccc4s3)CC2)c1]

Prepared through method C from intermediate 46 (0.25 g, 1.156 mmol) and 2-(chloromethyl)benzo[d]thiazole (0.318 g, 1.734 mmol). The alkylation reaction was completed after 6 h at 80° C. After performing an aq. work-up as described in method C, the obtained crude residue was purified by silica chromatography (40-45% EtOAc in hexane) to afford 3-[4-(1,3-benzothiazol-2-ylmethyl)piperazin-1-yl]-5-ethyl-pyridine-2-carbonitrile as an off-white solid (0.210 g, 50%).

Final tetrazole reaction was performed, by mixing the nitrile (0.200 g, 0.550 mmol) with sodium azide (0.286 g, 4.402 mmol) and Bu₃SnCl (1.433 g, 4.402 mmol) in toluene (20 mL) at 150° C. for 14 h in a sealed tube. After an aq. work-up and subsequent column chromatography over silica gel (6-8% MeOH in CH₂Cl₂), a final trituration using diethyl ether gave the envisaged title compound as a pale-yellow solid (29 mg, 13%).

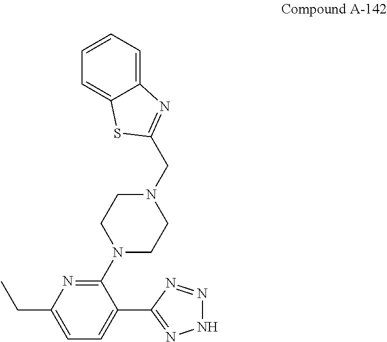 [SMILES: CCc1ccc(-c2nn[nH]n2)c(N2CCN(Cc3nc4ccccc4s3)CC2)n1]

Prepared through method C from intermediate 42 (0.080 g, 0.317 mmol) and 2-(chloromethyl)benzo[d]thiazole (0.087 g, 0.475 mmol). The alkylation reaction was completed after 6 h at 85° C. After performing an aq. work-up, the organic residue was purified by silica chromatography (30-40% EtOAc in hexane) to give 2-[4-(1,3-benzothiazol-2-ylmethyl)piperazin-1-yl]-6-ethyl-pyridine-3-carbonitrile as a viscous liquid (90 mg, 78%).

Final tetrazole reaction, followed by aq. work-up, column chromatography (5-8% MeOH in CH₂Cl₂) and ultimate trituration of the purified compound with diethyl ether, yielded the desired compound as an off-white solid (13 mg, 13%).

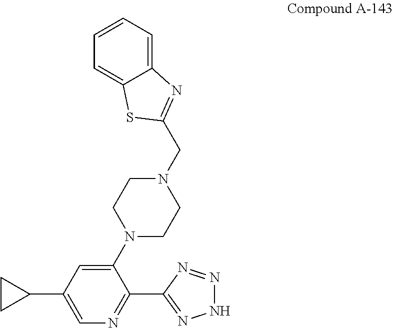 [SMILES: c1ccc2sc(CN3CCN(c4cc(C5CC5)cnc4-c4nn[nH]n4)CC3)nc2c1]

Prepared through method C from intermediate 45 (0.150 g, 0.657 mmol) and 2-(chloromethyl)benzo[d]thiazole (0.181 g, 0.986 mmol). The alkylation reaction was completed after 6 h at 80° C. After performing an aq. work-up as described in method C, the obtained crude residue was purified by silica chromatography (40-45% EtOAc in hexane) to afford 3-[4-(1,3-benzothiazol-2-ylmethyl)piperazin-1-yl]-5-cyclopropyl-pyridine-2-carbonitrile as an off-white solid (0.192 g, 78%).

Final tetrazole reaction was performed by mixing the nitrile (0.190 g, 0.506 mmol) with sodium azide (0.263 g, 4.048 mmol) and Bu₃SnCl (1.318 g, 4.048 mmol) in toluene (20 mL) at 150° C. for 14 h in a sealed tube. After an aq. work-up and subsequent column chromatography over silica gel (6-8% MeOH in CH₂Cl₂), followed by a final trituration using diethyl ether, gave the envisaged title compound as an off-white solid (24 mg, 11%).

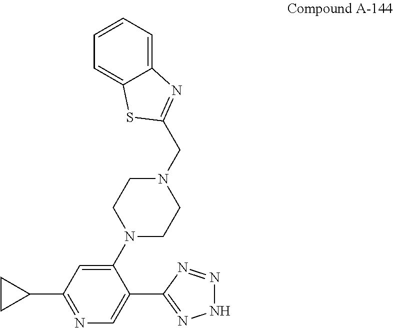 [SMILES: c1ccc2sc(CN3CCN(c4cc(C5CC5)ncc4-c4nn[nH]n4)CC3)nc2c1]

Prepared through method C from intermediate 44 (0.180 g, 0.788 mmol) and 2-(chloromethyl)benzo[d]thiazole (0.217 g, 1.183 mmol). The alkylation reaction was completed after 6 h at 85° C. After performing an aq. work-up as described in method C, the organic residue was purified by silica chromatography (30-40% EtOAc in hexane) to afford 4-[4-(1,3-benzothiazol-2-ylmethyl)piperazin-1-yl]-6-cyclopropyl-pyridine-3-carbonitrile as a pale-yellow liquid (0.142 g, 48%).

Final tetrazole reaction was performed, by mixing the nitrile (0.140 g, 0.373 mmol) with sodium azide (0.194 g, 2.983 mmol) and Bu₃SnCl (0.971 g, 2.983 mmol) in toluene (10 mL) at 150° C. for 14 h in a sealed tube. Aq. work-up, followed by column chromatography (5-8% MeOH in CH₂Cl₂) and ultimate trituration of the purified compound with diethyl ether yielded the desired substituted tetrazole as an off-white solid (16 mg, 10%).

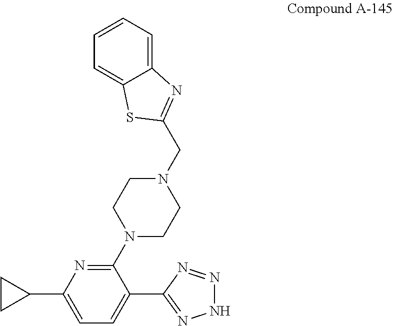 [SMILES: c1ccc2sc(CN3CCN(c4nc(C5CC5)ccc4-c4nn[nH]n4)CC3)nc2c1]

Prepared through method C from intermediate 41 (0.300 g, 1.314 mmol) and 2-(chloromethyl)benzo[d]thiazole (0.362 g, 1.971 mmol). The alkylation reaction was completed after 6 h at 85° C. After performing an aq. work-up as described in method C, the crude organic residue was purified by silica chromatography (30-40% EtOAc in hexane) to afford 2-[4-(1,3-benzothiazol-2-ylmethyl)piperazin-1-yl]-6-cyclopropyl-pyridine-3-carbonitrile as an off-white solid (0.210 g, 42%).

Final tetrazole reaction was performed by mixing the nitrile (0.200 g, 0.533 mmol) with sodium azide (0.277 g, 4.261 mmol) and Bu₃SnCl (1.387 g, 4.261 mmol) in toluene (10 mL) at 150° C. for 14 h in a sealed tube. After an aq. work-up and subsequent column chromatography over silica gel (6-8% MeOH in CH₂Cl₂), a trituration of the purified compound using diethyl ether yielded the desired substituted tetrazole as an off-white solid (25 mg, 11%).

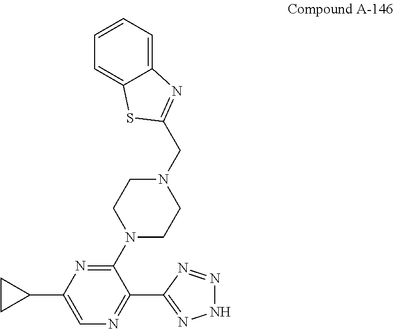 [SMILES: c1ccc2sc(CN3CCN(c4nc(C5CC5)cnc4-c4nn[nH]n4)CC3)nc2c1]

Prepared through method C from intermediate 48 (0.250 g, 1.090 mmol) and 2-(chloromethyl)benzo[d]thiazole (0.300 g, 1.636 mmol). The alkylation reaction was completed after 6 h at 80° C. After performing an aq. work-up as described in method C, the obtained crude residue was purified by silica chromatography (30-40% EtOAc in hexane) to afford 3-[4-(1,3-benzothiazol-2-ylmethyl)piperazin-1-yl]-5-cyclopropyl-pyrazine-2-carbonitrile as an off-white solid (0.185 g, 45%).

Final tetrazole reaction was performed, by mixing the nitrile (0.180 g, 0.478 mmol) with sodium azide (0.249 g, 3.825 mmol) and Bu₃SnCl (1.245 g, 3.825 mmol) in toluene (20 mL) at 160° C. for 14 h in a sealed tube. After an aq. work-up and subsequent column chromatography over silica gel (6-8% MeOH in CH₂Cl₂), a final trituration using diethyl ether gave the envisaged title compound as a yellow solid (24 mg, 12%).

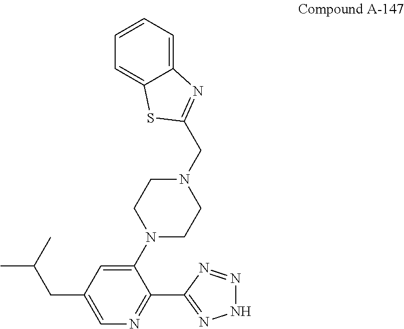 [SMILES: CC(C)Cc1cnc(-c2nn[nH]n2)c(N2CCN(Cc3nc4ccccc4s3)CC2)c1]

Prepared through method C from intermediate 47 (0.080 g, 0.285 mmol) and 2-(chloromethyl)benzo[d]thiazole (0.078 g, 0.427 mmol). The alkylation reaction was completed after 6 h at 80° C. After performing an aq. work-up as described in method C, the organic residue was purified by silica chromatography (40-45% EtOAc in hexane) to afford 3-[4-(1,3-benzothiazol-2-ylmethyl)piperazin-1-yl]-5-isobutyl-pyridine-2-carbonitrile as an off-white solid (0.065 g, 58%).

Final tetrazole reaction was performed, by mixing the nitrile (0.060 g, 0.153 mmol) with sodium azide (0.080 g, 1.226 mmol) and Bu₃SnCl (0.399 g, 1.226 mmol) in toluene (20 mL) at 150° C. for 14 h in a sealed tube. Aq. work-up, followed by column chromatography (6-8% MeOH in CH₂Cl₂) and ultimate trituration of the purified compound with diethyl ether, yielded the desired substituted tetrazole as a pale-yellow solid (9 mg, 13%).

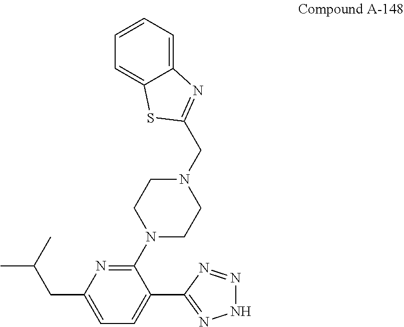 [SMILES: CC(C)Cc1ccc(-c2nn[nH]n2)c(N2CCN(Cc3nc4ccccc4s3)CC2)n1]

Prepared through method C from intermediate 43 (0.085 g, 0.321 mmol) and 2-(chloromethyl)benzo[d]thiazole (0.088 g, 0.481 mmol). The alkylation reaction was completed after 6 h at 85° C. After performing an aq. work-up as described in method C, the obtained crude residue was purified by silica chromatography (30-40% EtOAc in hexane) to give 2-[4-(1,3-benzothiazol-2-ylmethyl)piperazin-1-yl]-6-isobutyl-pyridine-3-carbonitrile as a gummy liquid (0.070 mg, 55%).

Final tetrazole reaction was performed, by mixing the nitrile (0.070 g, 0.179 mmol) with sodium azide (0.093 g, 1.43 mmol) and Bu₃SnCl (0.466 g, 1.43 mmol) in toluene (10 mL) at 150° C. for 14 h in a sealed tube. Aq. work-up as described in method C, followed by column chromatography (6-8% MeOH in CH₂Cl₂) and subsequent trituration of the purified compound with diethyl ether, afforded the desired substituted tetrazole as an off-white solid (8 mg, 10%).

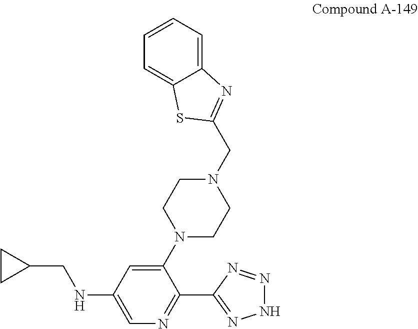 [SMILES: c1ccc2sc(CN3CCN(c4cc(NCC5CC5)cnc4-c4nn[nH]n4)CC3)nc2c1]

Prepared through method C from 5-(cyclopropylmethylamino)-3-piperazin-1-yl-pyridine-2-carbonitrile (intermediate 40, 0.22 g, 0.855 mmol) and 2-(chloromethyl)benzo[d]thiazole (0.236 g, 1.282 mmol). The alkylation reaction was completed after 6 h at 80° C. After performing an aq. work-up as described in method C, the obtained crude residue was purified by silica chromatography (40-50% EtOAc in hexane) to afford 3-[4-(1,3-benzothiazol-2-ylmethyl)piperazin-1-yl]-5-(cyclopropylmethylamino) pyridine-2-carbonitrile as an off-white solid (0.121 g, 35%).

Final tetrazole reaction was performed, by mixing the nitrile (0.121 g, 0.299 mmol) with sodium azide (0.156 g, 2.394 mmol) and Bu₃SnCl (0.779 g, 2.394 mmol) in toluene (10 mL) at 150° C. for 14 h in a sealed tube. After an aq. work-up and subsequent column chromatography over silica gel (6-8% MeOH in CH₂Cl₂), a final trituration using diethyl ether gave the envisaged title compound as an off-white solid (15 mg, 11%).

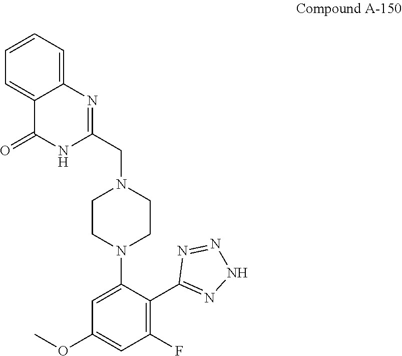 [SMILES: COc1cc(F)c(-c2nn[nH]n2)c(N2CCN(Cc3nc4ccccc4c(=O)[nH]3)CC2)c1]

Prepared through method M from 2-fluoro-4-methoxy-6-piperazin-1-yl-benzonitrile (intermediate 21, 0.20 g, 0.850 mmol) and 2-(chloromethyl)quinazolin-4-(3H)-one (0.248 g, 1.275 mmol). The alkylation reaction was completed after 6 h at 80° C. After performing an aq. work-up, the organic residue was purified by silica chromatography (50-60% EtOAc in hexane) to afford 2-fluoro-4-methoxy-6-(4-((4-oxo-3,4-dihydroquinazolin-2-yl)methyl)piperazin-1-yl)benzonitrile as an off-white solid (0.24 g, 72%).

Final tetrazole reaction was performed, by mixing the nitrile (0.240 g, 0.61 mmol), sodium azide (0.317 g, 4.88 mmol) and Bu₃SnCl (1.589 g, 4.88 mmol) in toluene (30 mL) at 150° C. for 14 h in a sealed tube. Aq. work-up as described in method M, followed by column chromatography (5-8% MeOH in CH₂Cl₂) and final trituration of the purified compound with diethyl ether, yielded the desired tetrazole as an off-white solid (60 mg, 23%).

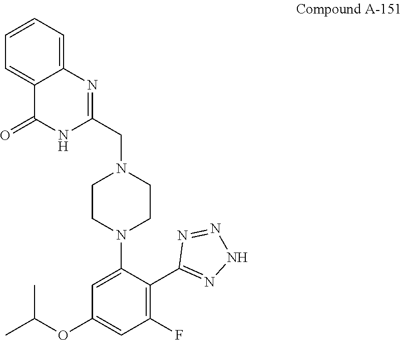 [SMILES: CC(C)Oc1cc(F)c(-c2nn[nH]n2)c(N2CCN(Cc3nc4ccccc4c(=O)[nH]3)CC2)c1]

Prepared through method M from intermediate 21 (0.20 g, 0.760 mmol) and 2-(chloromethyl)quinazolin-4-(3H)-one (0.222 g, 1.14 mmol). The alkylation reaction was completed after 6 h at 80° C. After performing an aq. work-up as described in method M, the organic residue was purified by silica chromatography (30-40% EtOAc in hexane) to afford 2-fluoro-4-isopropoxy-6-(4-((4-oxo-3,4-dihydroquinazolin-2-yl)methyl)piperazin-1-yl)benzonitrile as an off-white solid (0.25 g, 78%).

Final tetrazole reaction was performed by mixing the nitrile (0.24 g, 0.57 mmol), sodium azide (0.296 g, 4.56 mmol) and Bu₃SnCl (1.48 g, 4.56 mmol) in toluene (20 mL) at 150° C. for 14 h in a sealed tube. Aq. work-up, followed by column chromatography (5-8% MeOH in CH₂Cl₂) and subsequent trituration of the purified compound with diethyl ether, yielded the envisaged tetrazole A-151 as an off-white solid (20 mg, 8%).

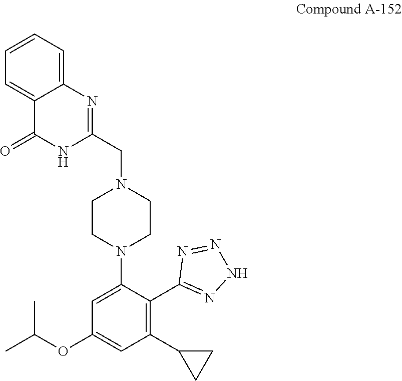 [SMILES: CC(C)Oc1cc(C2CC2)c(-c2nn[nH]n2)c(N2CCN(Cc3nc4ccccc4c(=O)[nH]3)CC2)c1]

Prepared through method M from 2-cyclopropyl-4-isopropoxy-6-(piperazin-1-yl)-benzonitrile (intermediate 25, 0.20 g, 0.70 mmol) and 2-(chloromethyl)quinazolin-4-(3H)-one (0.205 g, 1.05 mmol). The alkylation reaction was completed after 6 h at 80° C. After performing an aq. work-up, the crude organic residue was purified by silica chromatography (40-50% EtOAc in hexane) to afford 2-cyclopropyl-4-isopropoxy-6-(4-((4-oxo-3,4-dihydroquinazolin-2-yl)methyl)piperazin-1-yl)benzonitrile as an off-white solid (0.143 g, 46%).

Final tetrazole reaction was performed, by mixing the nitrile (0.09 g, 0.203 mmol), sodium azide (0.106 g, 1.62 mmol) and Bu₃SnCl (0.528 g, 1.62 mmol) in toluene (10 mL) at 150° C. for 14 h in a sealed tube. Aq. work-up as described in method M, followed by column chromatography (5-8% MeOH in CH₂Cl₂) and final trituration with diethyl ether, afforded the envisaged substituted tetrazole as an off-white solid (6 mg, 6%).

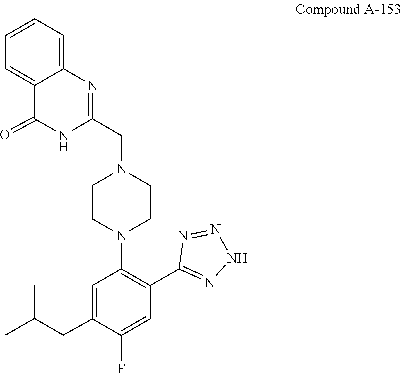 [SMILES: CC(C)Cc1cc(N2CCN(Cc3nc4ccccc4c(=O)[nH]3)CC2)c(-c2nn[nH]n2)cc1F]

Prepared through method M from intermediate 29 (0.10 g, 0.336 mmol) and 2-(chloromethyl)quinazolin-4-(3H)-one (0.098 g, 0.504 mmol). The alkylation reaction was completed after 6 h at 80° C. After performing an aq. work-up, the crude organic residue was purified by silica chromatography (45-50% EtOAc in hexane) to afford 5-fluoro-4-isobutyl-2-(4-((4-oxo-3,4-dihydroquinazolin-2-yl)methyl)piperazin-1-yl)benzonitrile as an off-white solid (0.065 g, 46%).

Final tetrazole reaction was performed, by mixing the nitrile (0.05 g, 0.119 mmol), sodium azide (0.062 g, 0.954 mmol) and Bu₃SnCl (0.310 g, 0.954 mmol) in toluene (10 mL) at 150° C. for 14 h in a sealed tube. Aq. work-up as described in method M, followed by column chromatography (5-8% MeOH in CH₂Cl₂) and final trituration with diethyl ether, afforded the envisaged substituted tetrazole as an off-white solid (7 mg, 13%).

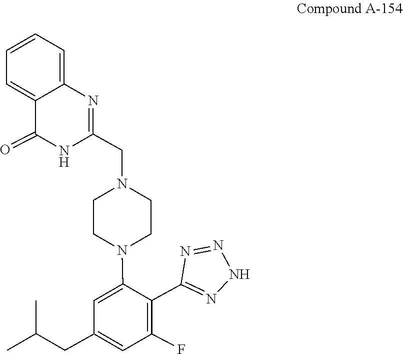 [SMILES: CC(C)Cc1cc(F)c(-c2nn[nH]n2)c(N2CCN(Cc3nc4ccccc4c(=O)[nH]3)CC2)c1]

To an ice-cold solution of intermediate 4 (0.24 g, 0.808 mmol) in DMF (10 mL) was added triethylamine (0.34 mL, 2.42 mmol) followed by 2-(chloromethyl)quinazolin-4-(3H)-one (0.235 g, 1.212 mmol) after which the reaction mixture was stirred at 80° C. for 6 h. Upon confirmation of the completion of the reaction by TLC, the mixture was diluted with water and extracted with ethyl acetate (2×30 mL). The combined organic layers were washed with water, sat. brine, dried over anhydrous sodium sulfate and evaporated under reduced pressure to afford the crude compound. Purification by column chromatography over silica gel (40-50% of ethyl acetate in hexane) afforded 2-fluoro-4-isobutyl-6-(4-((4-oxo-3,4-dihydroquinazolin-2-yl)methyl)piperazin-1-yl)benzonitrile as an off-white solid (0.225 g, 66%).

To a stirred solution of 2-fluoro-4-isobutyl-6-(4-((4-oxo-3,4-dihydroquinazolin-2-yl)methyl)piperazin-1-yl)benzonitrile (0.22 g, 0.525 mmol) in toluene (15 mL) was added sodium azide (0.273 g, 4.20 mmol) and Bu₃SnCl (1.365 g, 4.20 mmol), after which the sealed tube containing the reaction mixture was stirred at 150° C. for 14 h. When complete conversion was observed (via TLC), the reaction mixture was concentrated under reduced pressure. The obtained residue was re-dissolved in CH₂Cl₂ and was washed with a 10% NaOH solution. The aqueous layer was then neutralized with a citric acid solution and was extracted with CH₂Cl₂ (2×50 mL). The combined organic layers were washed with sat. brine, dried over anhydrous sodium sulfate and evaporated under reduced pressure to afford the crude residue. The crude compound was purified by silica chromatography (5-8% of MeOH in CH₂Cl₂), followed by an additional trituration with diethyl ether. The desired compound was obtained as an off-white solid with enriched purity (39 mg, 16%).

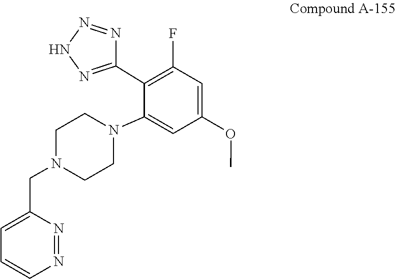 [SMILES: COc1cc(F)c(-c2nn[nH]n2)c(N2CCN(Cc3cccnn3)CC2)c1]

Prepared through method D from 2-fluoro-4-methoxy-6-piperazin-1-yl-benzonitrile (intermediate 21, 0.20 g, 0.851 mmol) and 3-(chloromethyl)pyridazine (0.163 g, 1.276 mmol). The alkylation reaction was completed after 6 h at 80° C. After performing an aq. work-up, the organic residue was purified by silica chromatography (50-60% EtOAc in hexane) to afford 2-fluoro-4-methoxy-6-(4-(pyridazin-3-ylmethyl)piperazin-1-yl)benzonitrile as an off-white solid (0.15 g, 54%).

Final tetrazole reaction was performed, by mixing the nitrile (0.14 g, 0.428 mmol), sodium azide (0.222 g, 3.425 mmol) and Bu₃SnCl (1.11 g, 3.425 mmol) in toluene (30 mL) at 150° C. for 14 h in a sealed tube. Aq. work-up as described in method D, followed by column chromatography (5-8% MeOH in CH₂Cl₂) and final trituration of the purified compound with diethyl ether, yielded the desired tetrazole as an off-white solid (11 mg, 10%).

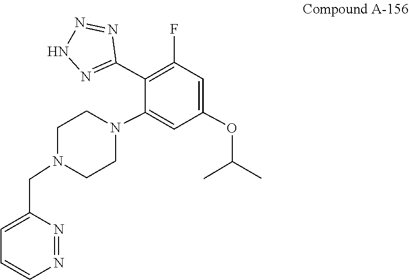 [SMILES: CC(C)Oc1cc(F)c(-c2nn[nH]n2)c(N2CCN(Cc3cccnn3)CC2)c1]

Prepared through method D from intermediate 21 (0.08 g, 0.304 mmol) and 3-(chloromethyl)pyridazine (0.06 g, 0.456 mmol). The alkylation reaction was completed after 6 h at 80° C. After performing an aq. work-up as described in method D, the organic residue was purified by silica chromatography (30-40% EtOAc in hexane) to afford 2-fluoro-4-isopropoxy-6-(4-(pyridazin-3-ylmethyl)piperazin-1-yl)benzonitrile as an off-white solid (0.075 g, 65%).

Final tetrazole reaction was performed, by mixing the nitrile (0.075 g, 0.57 mmol), sodium azide (0.296 g, 4.56 mmol) and Bu₃SnCl (1.48 g, 4.56 mmol) in toluene (20 mL) at 150° C. for 14 h in a sealed tube. Aq. work-up, followed by column chromatography (5-8% MeOH in CH₂Cl₂) and subsequent trituration of the purified compound with diethyl ether, yielded the envisaged tetrazole A-156 as an off-white solid (20 mg, 7%).

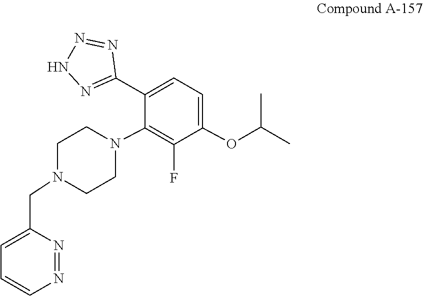 [SMILES: CC(C)Oc1ccc(-c2nn[nH]n2)c(N2CCN(Cc3cccnn3)CC2)c1F]

Prepared through method D from 3-fluoro-4-isopropoxy-2-(piperazin-1-yl)benzonitrile (intermediate 38, 0.28 g, 1.063 mmol) and 3-(chloromethyl)pyridazine (0.205 g, 1.595 mmol). The alkylation reaction was completed after 6 h at 80° C. After performing an aq. work-up as described in method D, the organic residue was purified by silica chromatography (40-45% EtOAc in hexane) to afford 3-fluoro-4-isopropoxy-2-(4-(pyridazin-3-ylmethyl)piperazin-1-yl)benzonitrile as an off-white solid (0.18 g, 48%).

Final tetrazole reaction was performed, by mixing the nitrile (0.18 g, 0.507 mmol), sodium azide (0.263 g, 4.052 mmol) and Bu₃SnCl (1.31 g, 4.052 mmol) in toluene (15 mL) at 150° C. for 14 h in a sealed tube. Aq. work-up as described in method D, followed by column chromatography (5-8% MeOH in CH₂Cl₂) and final trituration of the purified compound with diethyl ether, yielded the desired tetrazole as an off-white solid (20 mg, 10%).

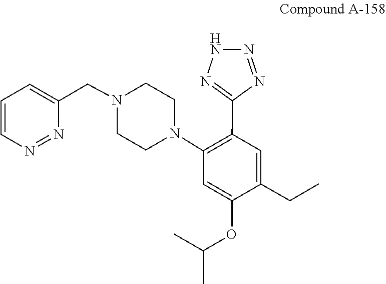 [SMILES: CCc1cc(-c2nn[nH]n2)c(N2CCN(Cc3cccnn3)CC2)cc1OC(C)C]

Prepared through method D from 5-ethyl-4-isopropoxy-2-piperazin-1-yl-benzonitrile (intermediate 24, 0.10 g, 0.36 mmol) and 3-(chloromethyl)pyridazine (0.07 g, 0.54 mmol). The alkylation reaction was completed after 6 h at 80° C. After performing an aq. work-up as described in method D, the organic residue was purified by silica chromatography (40-50% EtOAc in hexane) to afford 5-ethyl-4-isopropoxy-2-(4-(pyridazin-3-ylmethyl)piperazin-1-yl)benzonitrile as a pale-yellow gum (0.07 g, 53%).

Final tetrazole reaction was performed, by mixing the nitrile (0.07 g, 0.191 mmol) with sodium azide (0.100 g, 1.534 mmol) and Bu₃SnCl (0.498 g, 1.534 mmol) in toluene (10 mL) at 150° C. for 14 h in a sealed tube. Aq. work-up, followed by column chromatography (5-8% MeOH in CH₂Cl₂) and ultimate trituration of the purified compound with diethyl ether, yielded the desired substituted tetrazole as an off-white solid (8 mg, 10%).

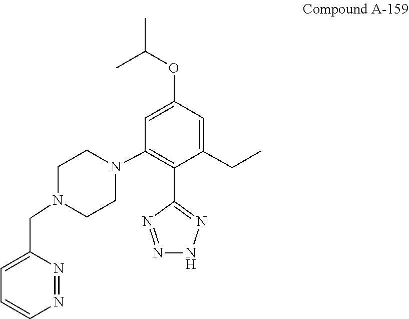 [SMILES: CCc1cc(OC(C)C)cc(N2CCN(Cc3cccnn3)CC2)c1-c1nn[nH]n1]

Prepared through method D from 2-ethyl-4-isopropoxy-6-piperazin-1-yl-benzonitrile hydrochloride (intermediate 26, 0.14 g, 0.45 mmol) and 3-(chloromethyl)pyridazine (0.087 g, 0.675 mmol). The alkylation reaction was completed after 6 h at 80° C. After performing an aq. work-up as described in method D, the organic residue was purified by silica chromatography (40-50% EtOAc in hexane) to afford 2-ethyl-4-isopropoxy-6-(4-(pyridazin-3-ylmethyl)piperazin-1-yl)benzonitrile as an off-white solid (0.12 g, 64%).

Final tetrazole reaction was performed, by mixing the nitrile (0.085 g, 0.20 mmol) with sodium azide (0.105 g, 1.60 mmol) and Bu₃SnCl (0.52 g, 1.60 mmol) in toluene (10 mL) at 150° C. for 14 h in a sealed tube. Aq. work-up as described in method D, followed by column chromatography (5-8% MeOH in CH₂Cl₂) and final trituration using diethyl ether, delivered the envisaged target compound A-159 as an off-white solid (10 mg, 14%).

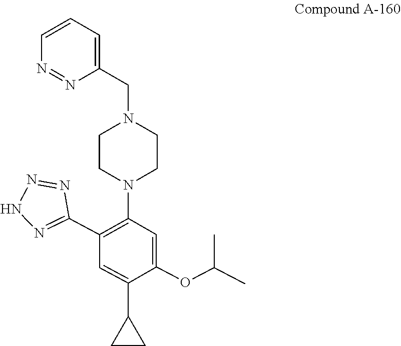 [SMILES: CC(C)Oc1cc(N2CCN(Cc3cccnn3)CC2)c(-c2nn[nH]n2)cc1C1CC1]

Prepared through method D from 3-cyclopropyl-4-isopropoxy-6-(piperazin-1-yl)benzonitrile (intermediate 23, 0.103 g, 0.36 mmol) and 3-(chloromethyl)pyridazine (0.070 g, 0.54 mmol). The alkylation reaction was completed after 6 h at 80° C. After performing an aq. work-up as described in method D, the organic residue was purified by silica chromatography (40-50% EtOAc in hexane) to afford 5-cyclopropyl-4-isopropoxy-2-(4-(pyridazin-3-ylmethyl)piperazin-1-yl)benzonitrile as a pale-yellow gum (0.09 g, 66%).

Final tetrazole reaction was performed, by mixing the nitrile (0.08 g, 0.212 mmol), sodium azide (0.11 g, 1.697 mmol) and Bu₃SnCl (0.55 g, 1.697 mmol) in toluene (10 mL) at 150° C. for 14 h in a sealed tube. Aq. work-up, followed by column chromatography (5-8% MeOH in CH₂Cl₂) and final trituration with diethyl ether yielded the desired title compound A-160 as an off-white solid (11 mg, 13%).

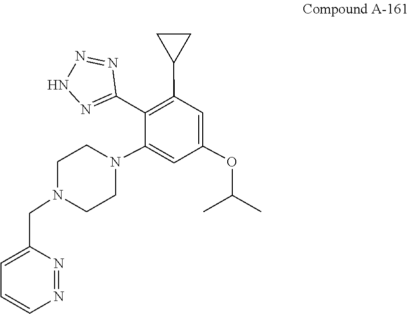 [SMILES: CC(C)Oc1cc(C2CC2)c(-c2nn[nH]n2)c(N2CCN(Cc3cccnn3)CC2)c1]

Prepared through method D from 2-cyclopropyl-4-isopropoxy-6-(piperazin-1-yl)-benzonitrile (intermediate 25, 0.15 g, 0.526 mmol) and 3-(chloromethyl)pyridazine (0.101 g, 0.789 mmol). The alkylation reaction was completed after 6 h at 80° C. After performing an aq. work-up, the crude organic residue was purified by silica chromatography (40-50% EtOAc in hexane) to afford 2-cyclopropyl-4-isopropoxy-6-(4-(pyridazin-3-ylmethyl)piperazin-1-yl)benzonitrile as an off-white solid (0.08 g, 40%).

Final tetrazole reaction was performed, by mixing the nitrile (0.08 g, 0.212 mmol), sodium azide (0.110 g, 1.69 mmol) and Bu₃SnCl (0.549 g, 1.69 mmol) in toluene (10 mL) at 150° C. for 14 h in a sealed tube. Aq. work-up as described in method D, followed by column chromatography (5-8% MeOH in CH₂Cl₂) and final trituration with diethyl ether, afforded the envisaged substituted tetrazole as an off-white solid (12 mg, 13%).

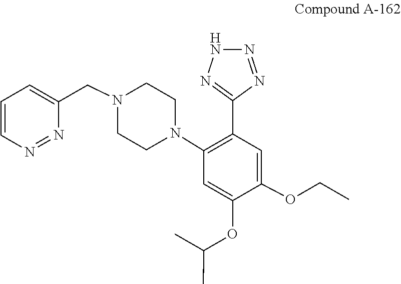 [SMILES: CCOc1cc(-c2nn[nH]n2)c(N2CCN(Cc3cccnn3)CC2)cc1OC(C)C]

Prepared through method D from 5-ethoxy-4-isopropoxy-2-(piperazin-1-yl)benzonitrile hydrochloride (intermediate 22, 0.12 g, 0.369 mmol) and 3-(chloromethyl)pyridazine (0.071 g, 0.554 mmol). The alkylation reaction was completed after 6 h at 80° C. After performing an aq. work-up, the organic residue was purified by silica chromatography (40-50% EtOAc in hexane) to afford 5-ethoxy-4-isopropoxy-2-(4-(pyridazin-3-ylmethyl)piperazin-1-yl)benzonitrile as an off-white solid (0.12 g, 85%).

Final tetrazole reaction was performed, by mixing the nitrile (0.12 g, 0.314 mmol), sodium azide (0.163 g, 2.513 mmol) and Bu₃SnCl (0.816 g, 2.513 mmol) in toluene (10 mL) at 150° C. for 14 h in a sealed tube. Aq. work-up as described in method D, followed by column chromatography (5-8% MeOH in CH₂Cl₂) and subsequent trituration of the purified compound with diethyl ether, yielded the desired substituted tetrazole as an off-white solid (15 mg, 10%).

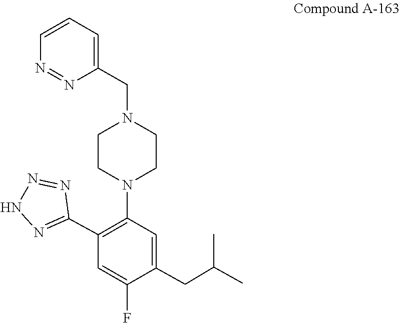 [SMILES: CC(C)Cc1cc(N2CCN(Cc3cccnn3)CC2)c(-c2nn[nH]n2)cc1F]

Prepared through method D from intermediate 29 (0.10 g, 0.336 mmol) and 3-(chloromethyl)pyridazine (0.070 g, 0.54 mmol). The alkylation reaction was completed after 6 h at 80° C. After performing an aq. work-up as described in method D, the obtained crude residue was purified by silica chromatography (45-50% EtOAc in hexane) to give 5-fluoro-4-isobutyl-2-(4-(pyridazin-3-ylmethyl)piperazin-1-yl)benzonitrile as an off-white solid (0.08 g, 69%).

Final tetrazole reaction was performed, by mixing the nitrile (0.070 g, 0.172 mmol), sodium azide (0.090 g, 1.372 mmol) and Bu₃SnCl (0.446 g, 1.372 mmol) in toluene (10 mL) at 150° C. for 14 h in a sealed tube. Aq. work-up as described in method D, followed by column chromatography (5-8% MeOH in CH₂Cl₂) and subsequent trituration of the purified compound with diethyl ether afforded the desired substituted tetrazole as an off-white solid (6 mg, 4%).

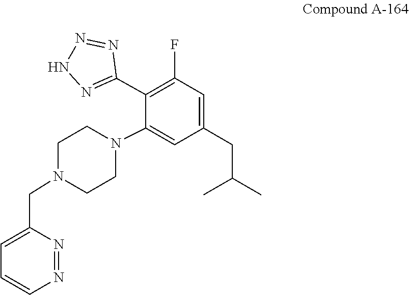 [SMILES: CC(C)Cc1cc(F)c(-c2nn[nH]n2)c(N2CCN(Cc3cccnn3)CC2)c1]

Prepared through method D from intermediate 4 (0.15 g, 0.505 mmol) and 3-(chloromethyl)pyridazine (0.096 g, 0.750 mmol). The alkylation reaction was completed after 6 h at 80° C. After performing an aq. work-up, the organic residue was purified by silica chromatography (40-50% EtOAc in hexane) to afford 2-fluoro-4-isobutyl-6-(4-(pyridazin-3-ylmethyl)piperazin-1-yl)benzonitrile as an off-white solid (0.150 g, 84%).

Final tetrazole reaction followed by aq. work-up, column chromatography (5-8% MeOH in CH₂Cl₂) and trituration of the purified compound with diethyl ether, yielded the desired compound as an off-white solid with enriched purity (18 mg, 12%).

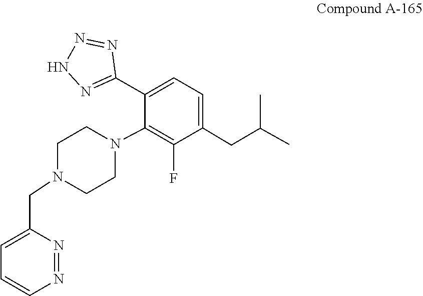 [SMILES: CC(C)Cc1ccc(-c2nn[nH]n2)c(N2CCN(Cc3cccnn3)CC2)c1F]

Prepared through method D from intermediate 39 (0.043 g, 0.146 mmol) and 3-(chloromethyl)pyridazine (0.028 g, 0.218 mmol). The alkylation reaction was completed after 6 h at 80° C. After performing an aq. work-up, the organic residue was purified by silica chromatography (2% MeOH in CH₂Cl₂) to afford 3-fluoro-4-isobutyl-2-(4-(pyridazin-3-ylmethyl)piperazin-1-yl)benzonitrile as an off-white solid (0.037 g, 72%). Final tetrazole reaction as described in method D, followed by aq. work-up, column chromatography (5-8% MeOH in CH₂Cl₂) and trituration of the purified compound with diethyl ether, yielded the desired compound A-165 as an off-white solid with enriched purity (12 mg, 29%).

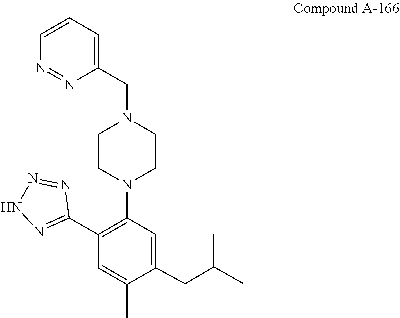 [SMILES: Cc1cc(-c2nn[nH]n2)c(N2CCN(Cc3cccnn3)CC2)cc1CC(C)C]

Prepared through method D from intermediate 31 (0.12 g, 0.404 mmol) and 3-(chloromethyl)pyridazine (0.078 g, 0.606 mmol). The alkylation reaction was completed after 6 h at 80° C. After performing an aq. work-up, the crude residue was purified by silica chromatography (45-50% EtOAc in hexane) to give 4-isobutyl-5-methyl-2-(4-(pyridazin-3-ylmethyl)piperazin-1-yl)benzonitrile as an off-white solid (0.14 g, 70%).

Final tetrazole reaction was performed, by mixing the nitrile (0.08 g, 0.229 mmol), sodium azide (0.119 g, 1.833 mmol) and Bu₃SnCl (0.595 g, 1.833 mmol) in toluene (10 mL) at 150° C. for 14 h in a sealed tube. After an aq. work-up and subsequent column chromatography (5-8% MeOH in CH₂Cl₂), an ultimate trituration of the purified compound with diethyl ether yielded the envisaged title compound A-166 as an off-white solid (12 mg, 8%).

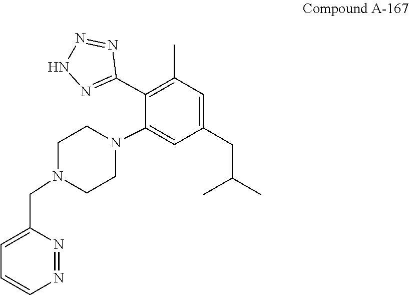 [SMILES: Cc1cc(CC(C)C)cc(N2CCN(Cc3cccnn3)CC2)c1-c1nn[nH]n1]

Prepared through method D from intermediate 5 (0.06 g, 0.204 mmol) and 3-(chloromethyl)pyridazine (0.040 g, 0.306 mmol). The alkylation reaction was completed after 6 h at 80° C. After performing an aq. work-up, the organic residue was purified by silica chromatography (40-50% EtOAc in hexane) to afford 4-isobutyl-2-methyl-6-(4-(pyridazin-3-ylmethyl)piperazin-1-yl)benzonitrile as an off-white solid (0.05 g, 65%).

Final tetrazole reaction followed by aq. work-up, column chromatography (5-8% MeOH in CH₂Cl₂) and ultimate trituration of the purified compound with diethyl ether, yielded the desired compound as a pale-yellow solid with enriched purity (10 mg, 13%).

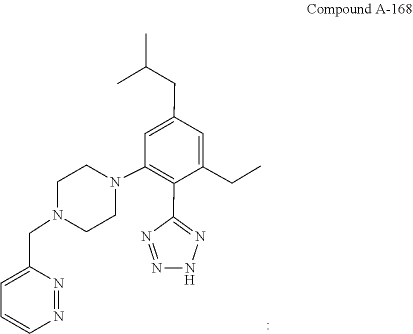

Prepared through method D from intermediate 9 (0.097 g, 0.360 mmol) and 3-(chloromethyl)pyridazine (0.070 g, 0.540 mmol). The alkylation reaction was completed after 6 h at 80° C. After performing an aq. work-up, the organic residue was purified by silica chromatography (50-55% EtOAc in hexane) to afford 2-ethyl-4-isobutyl-6-(4-(pyridazin-3-ylmethyl)piperazin-1-yl)benzonitrile as an off-white solid (0.108 g, 82%).

Final tetrazole reaction followed by aq. work-up, column chromatography (6-8% MeOH in CH₂Cl₂) and ultimate trituration of the purified compound with diethyl ether, yielded the desired compound as an off-white solid with enriched purity (12 mg, 11%).

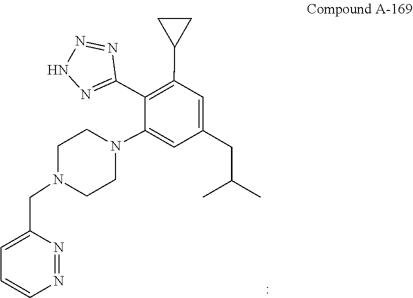 [SMILES: CC(C)Cc1cc(C2CC2)c(-c2nn[nH]n2)c(N2CCN(Cc3cccnn3)CC2)c1]

Prepared through method D from intermediate 8 (0.12 g, 0.376 mmol) and 3-(chloromethyl)pyridazine (0.072 g, 0.564 mmol). The alkylation reaction was completed after 6 h at 80° C. After performing an aq. work-up, the organic residue was purified by silica chromatography (50-55% EtOAc in hexane) to afford 2-cyclopropyl-4-isobutyl-6-(4-(pyridazin-3-ylmethyl)piperazin-1-yl)benzonitrile as an off-white solid (0.12 g, 82%).

Final tetrazole reaction followed by aq. work-up, column chromatography (6-8% MeOH in CH₂Cl₂) and ultimate trituration of the purified compound with diethyl ether, yielded the desired compound as an off-white solid with enriched purity (10 mg, 8%).

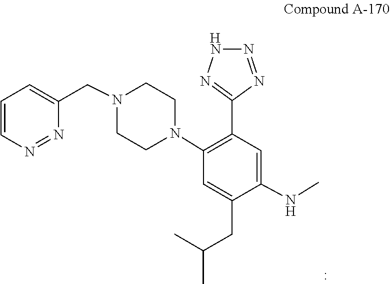

Prepared through method D from 4-isobutyl-5-(methylamino)-2-(piperazin-1-yl)benzonitrile dihydrochloride (intermediate 33, 0.18 g, 0.521 mmol) and 3-(chloromethyl)pyridazine (0.10 g, 0.781 mmol). The alkylation reaction was completed after 6 h at 80° C. After performing an aq. work-up as described in method D, the crude organic residue was purified by silica chromatography (45-50% EtOAc in hexane) to afford 4-isobutyl-5-(methylamino)-2-(4-(pyridazin-3-ylmethyl)piperazin-1-yl)benzonitrile as an off-white solid (0.084 g, 44%).

Final tetrazole reaction was performed, by mixing the nitrile (0.075 g, 0.206 mmol) with sodium azide (0.107 g, 1.648 mmol) and Bu₃SnCl (0.535 g, 1.648 mmol) in toluene (10 mL) at 150° C. for 14 h in a sealed tube. After an aq. work-up and subsequent column chromatography over silica gel (5-8% MeOH in CH₂Cl₂), a trituration of the purified compound with diethyl ether gave the targeted substituted tetrazole as an off-white solid (11 mg, 13%).

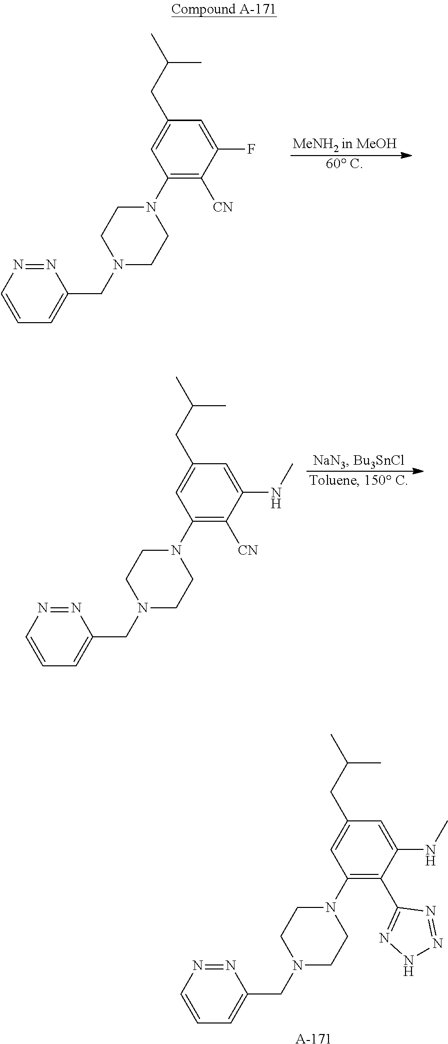 [SMILES: CC(C)Cc1cc(F)c(C#N)c(N2CCN(Cc3cccnn3)CC2)c1 ; CNc1cc(CC(C)C)cc(N2CCN(Cc3cccnn3)CC2)c1C#N ; CNc1cc(CC(C)C)cc(N2CCN(Cc3cccnn3)CC2)c1-c1nn[nH]n1 ; "A-171"]

To a solution of 2-fluoro-4-isobutyl-6-(4-(pyridazin-3-ylmethyl)piperazin-1-yl)benzonitrile (0.18 mg, 0.509 mmol) in methanol (2 mL) was added methyl amine in methanol (10 mL) and the resulting solution was stirred at 60° C. for 14 h. Upon completion of the reaction (by TLC), the reaction mixture was concentrated. The crude compound was purified by column chromatography over silica gel (3-4% MeOH in CH₂Cl₂) to afford the desired compound as a colorless liquid (0.13 g, 70%).

Final tetrazole reaction as described in method D using 4-isobutyl-2-(methylamino)-6-(4-(pyridazin-3-ylmethyl)piperazin-1-yl)benzonitrile (0.13 g, 0.357 mmol), sodium azide (0.185 g, 2.857 mmol) and Bu₃SnCl (0.928 g, 2.857 mmol) in toluene (10 mL) at 150° C., followed by aq. work-up, column chromatography (5-8% MeOH in CH₂Cl₂) and trituration of the purified compound with diethyl ether, yielded the desired compound as an off-white solid with enriched purity (14 mg, 10%).

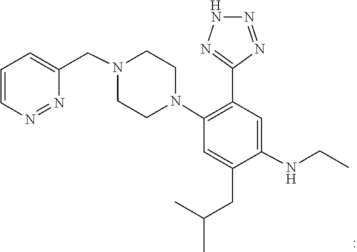

Prepared through method D from 5-(ethylamino)-4-isobutyl-2-(piperazin-1-yl)benzonitrile dihydrochloride (intermediate 33, 0.17 g, 0.473 mmol) and 3-(chloromethyl)pyridazine (0.091 g, 0.71 mmol). The alkylation reaction was completed after 6 h at 80° C. After performing an aq. work-up as described in method D, the obtained crude residue was purified by silica chromatography (45-50% EtOAc in hexane) to afford 5-(ethylamino)-4-isobutyl-2-(4-(pyridazin-3-ylmethyl)piperazin-1-yl)benzonitrile as an off-white solid (0.105 g, 59%).

Final tetrazole reaction was performed, by mixing the nitrile (0.100 g, 0.264 mmol) with sodium azide (0.137 g, 2.114 mmol) and Bu₃SnCl (0.688 g, 2.114 mmol) in toluene (15 mL) at 150° C. for 14 h in a sealed tube. After an aq. work-up and subsequent column chromatography over silica gel (5-8% MeOH in CH₂Cl₂), a final trituration using diethyl ether gave the envisaged title compound as an off-white solid (13 mg, 12%).

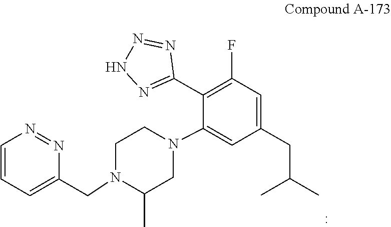 [SMILES: CC(C)Cc1cc(F)c(-c2nn[nH]n2)c(N2CCN(Cc3cccnn3)C(C)C2)c1]

Prepared through method G from intermediate 10. To a stirred solution of tert-butyl 3-methylpiperazine-1-carboxylate (300 mg, 1.50 mmol) in DMF (10 mL) was added Et₃N (0.63 mL, 4.50 mmol) at 0° C., after which the reaction was stirred at r.t. for 10 minutes.

Then 3-(chloromethyl)pyridazine (231 mg, 1.80 mmol) was added and the reaction was continued at r.t. for an additional 10 hours. After completion of the reaction was confirmed by TLC, the reaction mixture was worked up as described in method G. Subsequent column chromatography over silica gel (eluting with 2-3% MeOH in CH₂Cl₂) afforded tert-butyl 3-methyl-4-(pyridazin-3-ylmethyl)piperazine-1-carboxylate (208 mg, 47%).

To a stirred solution of tert-butyl 3-methyl-4-(pyridazin-3-ylmethyl)piperazine-1-carboxylate (200 mg, 0.68 mmol) in 1,4-dioxane (5 mL), HCl (g) in dioxane (20 mL) was added at 0° C. and the reaction was continued at r.t. for 5 h. After the completion of the reaction was confirmed by TLC, the reaction mixture was evaporated to dryness under reduced pressure. Next, the residue was washed with hexane to afford a crude 3-((2-methylpiperazin-1-yl)methyl)pyridazine hydrochloride, which was used as such in the next step without purification (154 mg crude).

To a stirred solution of 3-((2-methylpiperazin-1-yl)methyl)pyridazine hydrochloride (150 mg, 0.66 mmol) in DMF (10 mL) was added K₂CO₃ (227 mg, 1.64 mmol) at 0° C. and the reaction was stirred at r.t. for 10 minutes. Then 2,6-difluoro-4-isobutylbenzonitrile (141 mg, 0.72 mmol) was added at r.t. and the reaction was continued at 80° C. for 8 h. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The obtained crude residue was purified by column chromatography over silica gel (4-5% MeOH in CH₂Cl₂) to afford the envisaged product 2-fluoro-4-isobutyl-6-(3-methyl-4-(pyridazin-3-ylmethyl)piperazin-1-yl)benzonitrile (112 mg, 45% over 2 steps).

A mixture of this isolated nitrile (110 mg, 0.30 mmol), NaN₃ (156 mg, 2.39 mmol) and Bu₃SnCl (0.65 mL, 2.39 mmol) in toluene (10 mL) was stirred at 140° C. for 18 h in a sealed tube. After the completion of the reaction was confirmed by TLC, the reaction mixture was concentrated in vacuo. The residue was dissolved in CH₂Cl₂, washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The obtained crude was purified by silica chromatography (6-8% of MeOH in CH₂Cl₂), followed by trituration with diethyl ether to afford 3-[[4-[3-fluoro-5-isobutyl-2-(2H-tetrazol-5-yl)phenyl]-2-methyl-piperazin-1-yl]methyl]pyridazine as an off-white solid (6 mg, 5%).

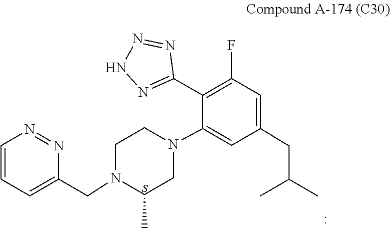

Prepared through method G from intermediate 10. To a stirred solution of N-Boc protected (S)-methyl piperazine (40 g, 200 mmol) in DMF (400 mL) was added DIPEA (87.1 mL, 500 mmol) at 0° C., after which the reaction was stirred at r.t. for 10 minutes. Then 3-(chloromethyl)pyridazine (30.8 g, 240 mmol) was added and the reaction was continued at 60° C. for 14 h. After completion of the reaction was confirmed by TLC, the reaction mixture was diluted with cold water and extracted with CH₂Cl₂. The combined organic layers were washed with sat. brine, dried over anhydrous sodium sulfate and evaporated under reduced pressure. The obtained crude residue was purified by column chromatography over silica gel (eluting with 2-3% MeOH in CH₂Cl₂) to afford (S)-tert-butyl 3-methyl-4-(pyridazin-3-ylmethyl)piperazine-1-carboxylate (35 g, 60%).

To the stirred solution of (S)-tert-butyl 3-methyl-4-(pyridazin-3-ylmethyl)piperazine-1-carboxylate (35 g, 119.86 mmol) in 1,4-dioxane (200 mL), HCl (g) in dioxane (200 mL) was added at 0° C. and the reaction was continued at r.t. for 5 h. After the completion of the reaction was confirmed by TLC, the reaction mixture was evaporated to dryness under reduced pressure, then washed with hexane to afford a crude residue (S)-3-((2-methylpiperazin-1-yl)methyl)pyridazine hydrochloride. The crude compound thus obtained was taken to the next step without purification (28 g crude).

To a stirred solution of (S)-3-((2-methylpiperazin-1-yl)methyl)pyridazine hydrochloride (28 g, 122.8 mmol) in DMF (300 mL) were added DIPEA (53.4 mL, 307.01 mmol) and K₂CO₃ (33.94 g, 245.6 mmol) at 0° C. and the reaction was stirred at r.t. for 10 minutes. Then 2,6-difluoro-4-isobutylbenzonitrile (23.97 g, 122.8 mmol) was added at r.t. and the reaction was continued at 65° C. for 14 h. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The crude thus obtained was purified by column chromatography over silica gel, eluting with 4-5% MeOH in CH₂Cl₂, to afford the envisaged product (S)-2-fluoro-4-isobutyl-6-(3-methyl-4-(pyridazin-3-ylmethyl)piperazin-1-yl)benzonitrile (34 g, 77% over 2 steps).

A mixture of this isolated nitrile (34 g, 92.64 mmol), NaN₃ (48.17 g, 741.14 mmol) and Bu₃SnCl (201 mL, 741.14 mmol) in toluene (350 mL) was stirred at 140° C. for 12 h in a sealed tube. After the completion of the reaction was confirmed by TLC, the reaction mixture was evaporated under reduced pressure. The residue was dissolved in CH₂Cl₂ and washed with a 10% NaOH solution. The aqueous layer was then neutralized with a citric acid solution and extracted with CH₂Cl₂ (3×250 mL). The combined organic layers were washed with sat. brine, dried over anhydrous sodium sulfate and evaporated under reduced pressure. The crude compound thus obtained was purified by silica chromatography (6-8% of MeOH in CH₂Cl₂) to afford 3-[[(2S)-4-[3-fluoro-5-isobutyl-2-(2H-tetrazol-5-yl)phenyl]-2-methyl-piperazin-1-yl]methyl]pyridazine which was triturated with diethyl ether to obtain a pale-yellow solid (15.5 g, 41%).

Two deuterated analogs of compound A-174 were also prepared (see the synthesis of compounds A-250 and A-251 below).

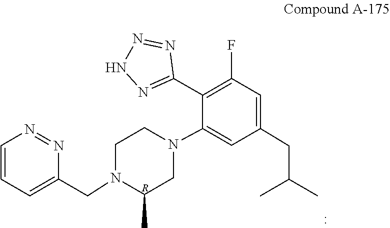

Prepared in a similar fashion as compound A-174 through method G by mixing N-Boc protected (R)-methyl piperazine (35.0 g, 174.8 mmol), DIPEA (91.3 mL, 524.3 mmol) and 3-(chloromethyl)pyridazine (26.9 g, 209.7 mmol) at 65° C. for 10 h. After performing an aq. work-up, the organic residue was purified by silica chromatography (2-3% MeOH in CH₂Cl₂) to give (R)-tert-butyl 3-methyl-4-(pyridazin-3-ylmethyl)piperazine-1-carboxylate (29.6 g, 58%).

Subsequent Boc deprotection of (R)-tert-butyl 3-methyl-4-(pyridazin-3-ylmethyl)piperazine-1-carboxylate (25.0 g, 85.5 mmol) in 1,4-dioxane (100 mL) using HCl (g) in dioxane (200 mL) delivered the targeted hydrochloride salt after 5 h stirring at r.t.

To a stirred solution of (R)-3-((2-methylpiperazin-1-yl)methyl)pyridazine hydrochloride (18.0 g, 78.7 mmol) in DMF (200 mL) were added DIPEA (34.3 mL, 196.7 mmol) and K₂CO₃ (27.2 g, 196.7 mmol) at 0° C., after which the reaction was stirred at r.t. for 10 minutes. Then 2,6-difluoro-4-isobutylbenzonitrile (16.9 g, 86.6 mmol) was added at r.t. and the reaction was continued at 65° C. for 14 h. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The crude thus obtained was purified by column chromatography over silica gel, eluting with 4-5% MeOH in CH₂Cl₂, to afford the envisaged product (R)-2-fluoro-4-isobutyl-6-(3-methyl-4-(pyridazin-3-ylmethyl)piperazin-1-yl)benzonitrile (17.5 g, 56% over 2 steps).

Final tetrazole reaction was performed, by mixing the nitrile (16.5 g, 44.9 mmol), NaN₃ (23.3 g, 359.2 mmol) and Bu₃SnCl (97.4 mL, 359.2 mmol) in toluene (350 mL) at 150° C. for 12 h in a sealed tube. Aq. work-up, followed by column chromatography (6-8% MeOH in CH₂Cl₂) and ultimate trituration of the purified compound with diethyl ether, yielded the envisaged tetrazole as an off-white solid (8.5 g, 46%).

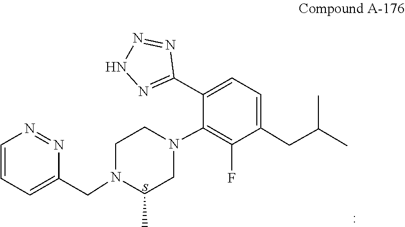

Prepared through method G from intermediate 50. To a stirred solution of (S)-3-((2-methylpiperazin-1-yl)methyl)pyridazine hydrochloride (128 mg, 0.559 mmol) in DMSO (5 mL) was added DIPEA (0.24 mL, 1.40 mmol) at 0° C. after which the reaction was stirred at r.t. for 10 minutes. Then intermediate 50 (120 mg, 0.615 mmol) was added at r.t. and the reaction was continued at 65° C. for 14 h. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The crude thus obtained was purified by column chromatography over silica gel, eluting with 2-3% MeOH in CH₂Cl₂, to afford the desired product as a gummy liquid (72 mg, 35%).

A mixture of this isolated nitrile (100 mg, 0.272 mmol), NaN₃ (142 mg, 2.18 mmol) and Bu₃SnCl (0.59 mL, 2.18 mmol) in toluene (10 mL) was stirred at 140° C. for 14 h in a sealed tube. After the completion of the reaction was confirmed by TLC, the reaction mixture was evaporated under reduced pressure. The residue was dissolved in CH₂Cl₂ and washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The obtained crude compound was purified by silica chromatography (4-5% of MeOH in CH₂Cl₂) and subsequent trituration in diethyl ether to afford 3-[[(2S)-4-[2-fluoro-3-isobutyl-6-(2H-tetrazol-5-yl)phenyl]-2-methyl-piperazin-1-yl]methyl]pyridazine as a colorless solid (13 mg, 12%).

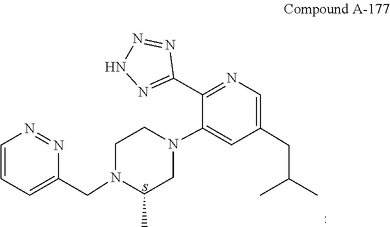

Prepared through method G from intermediate 49. To a stirred solution of N-Boc protected (S)-methyl piperazine (1.00 g, 4.99 mmol) in DMF (10 mL) was added DIPEA (2.61 mL, 14.9 mmol) at 0° C., after which the reaction was stirred at r.t. for 10 minutes. Then 3-(chloromethyl)pyridazine (770 mg, 5.99 mmol) was added and the reaction was continued at 60° C. for 14 h. After completion of the reaction was confirmed by TLC, the reaction mixture was diluted with cold water and extracted with CH₂Cl₂. The combined organic layers were washed with sat. brine, dried over anhydrous sodium sulfate and evaporated under reduced pressure. The obtained crude residue was purified by column chromatography over silica gel (eluting with 2-3% MeOH in CH₂Cl₂) to afford (S)-tert-butyl 3-methyl-4-(pyridazin-3-ylmethyl)piperazine-1-carboxylate (0.69 g, 47%).

To the stirred solution of (S)-tert-butyl 3-methyl-4-(pyridazin-3-ylmethyl)piperazine-1-carboxylate (400 mg, 1.37 mmol) in 1,4-dioxane (4 mL), HCl (g) in dioxane (4 mL) was added at 0° C. and the reaction was continued at r.t. for 5 h. After the completion of the reaction was confirmed by TLC, the reaction mixture was evaporated to dryness under reduced pressure, then washed with hexane to afford a crude residue (S)-3-((2-methylpiperazin-1-yl)methyl)pyridazine hydrochloride which was taken to the next step without additional purification (310 mg crude).

To a stirred solution of (S)-3-((2-methylpiperazin-1-yl)methyl)pyridazine hydrochloride (250 mg, 1.09 mmol) in DMF (5 mL) was added DIPEA (0.48 mL, 2.73 mmol) and K₂CO₃ (378 mg, 2.73 mmol) at 0° C. after which the reaction was stirred at r.t. for 10 minutes. Then intermediate 49 (214 mg, 1.20 mmol) was added at r.t. and the reaction was continued at 65° C. for 14 h. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The crude thus obtained was purified by column chromatography over silica gel eluting with 4-5% MeOH in CH₂Cl₂ to afford the envisaged product (278 mg, 58% over 2 steps).

A mixture of this isolated nitrile (100 mg, 0.285 mmol), NaN₃ (148 mg, 2.28 mmol) and Bu₃SnCl (0.62 mL, 2.28 mmol) in toluene (10 mL) was stirred at 140° C. for 14 h in a sealed tube. After the completion of the reaction was confirmed by TLC, the reaction mixture was evaporated under reduced pressure. The residue was dissolved in CH₂Cl₂ and washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The obtained crude compound was purified by silica chromatography (4-5% of MeOH in CH₂Cl₂) to afford 3-[[(2S)-4-[5-isobutyl-2-(2H-tetrazol-5-yl)-3-pyridyl]-2-methyl-piperazin-1-yl]methyl]pyridazine which was further triturated with diethyl ether to obtain a colorless solid (17 mg, 15%).

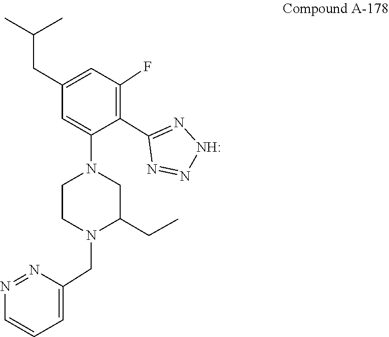

Prepared through method G from intermediate 10. To a stirred solution of tert-butyl 3-ethylpiperazine-1-carboxylate (250 mg, 1.17 mmol) in DMF (10 mL) was added DIPEA (0.61 mL, 3.50 mmol) at 0° C., after which the reaction was stirred at r.t. for 10 minutes. Then 3-(chloromethyl)pyridazine (180 mg, 1.40 mmol) was added and the reaction was continued at 60° C. for 14 h. After completion of the reaction was confirmed by TLC, the reaction mixture was worked up as described in method G. Subsequent column chromatography over silica gel (eluting with 3-4% MeOH in CH₂Cl₂) afforded tert-butyl 3-ethyl-4-(pyridazin-3-ylmethyl)piperazine-1-carboxylate (186 mg, 52%).

To a stirred solution of tert-butyl 3-ethyl-4-(pyridazin-3-ylmethyl)piperazine-1-carboxylate (180 mg, 0.59 mmol) in 1,4-dioxane (5 mL) was added HCl (g) in dioxane (25 mL) at 0° C. and the reaction was continued at r.t. for 5 h. After the completion of the reaction was confirmed by TLC, the reaction mixture was evaporated to dryness under reduced pressure. Next, the residue was washed with hexane to afford a crude 3-((2-ethylpiperazin-1-yl)methyl)pyridazine hydrochloride, which was used as such in the next step (133 mg crude).

To a stirred solution of 3-((2-ethylpiperazin-1-yl)methyl)pyridazine hydrochloride (130 mg, 0.54 mmol) in DMF (10 mL) was added DIPEA (0.24 mL, 1.34 mmol) and K₂CO₃ (185 mg, 1.34 mmol) at 0° C. after which the reaction was stirred at r.t. for 10 minutes. Then 2,6-difluoro-4-isobutylbenzonitrile (115 mg, 0.59 mmol) was added at r.t. after which the reaction was continued at 60° C. for 13 h. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The obtained crude residue was purified by column chromatography over silica gel (3-5% MeOH in CH₂Cl₂) to afford the envisaged 2-(3-ethyl-4-(pyridazin-3-ylmethyl)piperazin-1-yl)-6-fluoro-4-isobutylbenzonitrile (113 mg, 50% over 2 steps).

A mixture of this isolated nitrile (110 mg, 0.29 mmol), NaN₃ (150 mg, 2.31 mmol) and Bu₃SnCl (0.63 mL, 2.31 mmol) in toluene (10 mL) was stirred at 140° C. for 16 h in a sealed tube. After the completion of the reaction was confirmed by TLC, the reaction mixture was concentrated in vacuo. The residue was dissolved in CH₂Cl₂ and washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The crude compound thus obtained was purified by silica chromatography (6-8% of MeOH in CH₂Cl₂), followed by trituration with diethyl ether to afford 3-[[2-ethyl-4-[3-fluoro-5-isobutyl-2-(2H-tetrazol-5-yl)phenyl]piperazin-1-yl]methyl]pyridazine as an off-white solid (7 mg, 6%).

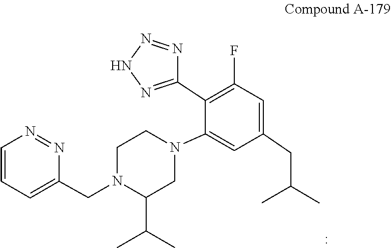 [SMILES: CC(C)Cc1cc(F)c(-c2nn[nH]n2)c(N2CCN(Cc3cccnn3)C(C(C)C)C2)c1]

Prepared through method G from intermediate 10. To a stirred solution of tert-butyl 3-isopropylpiperazine-1-carboxylate (150 mg, 0.66 mmol) in DMF (10 mL) was added DIPEA (0.35 mL, 1.97 mmol) at 0° C., after which the reaction was stirred at r.t. for 10 minutes. Then 3-(chloromethyl)pyridazine (101 mg, 0.79 mmol) was added and the reaction was continued at 60° C. for 14 h. After completion of the reaction was confirmed by TLC, the reaction mixture was worked up as described in method G. Subsequent column chromatography over silica gel (eluting with 3-4% MeOH in CH₂Cl₂) afforded tert-butyl 3-isopropyl-4-(pyridazin-3-ylmethyl)piperazine-1-carboxylate (88 mg, 42%).

To a stirred solution of tert-butyl 3-isopropyl-4-(pyridazin-3-ylmethyl)piperazine-1-carboxylate (80 mg, 0.25 mmol) in 1,4-dioxane (5 mL) was added HCl (g) in dioxane (15 mL) at 0° C. and the reaction was continued at r.t. for 5 h. After the completion of the reaction was confirmed by TLC, the reaction mixture was evaporated to dryness under reduced pressure. Next, the residue was washed with hexane to afford a crude 3-((2-isopropylpiperazin-1-yl)methyl)pyridazine hydrochloride, which was used as such in the next step (48 mg crude).

To a stirred solution of 3-((2-isopropylpiperazin-1-yl)methyl)pyridazine hydrochloride (45 mg, 0.18 mmol) in DMF (10 mL) was added DIPEA (0.08 mL, 0.44 mmol) and K₂CO₃ (61 mg, 0.44 mmol) at 0° C. after which the reaction was stirred at r.t. for 10 minutes. Then, 2,6-difluoro-4-isobutylbenzonitrile (38 mg, 0.19 mmol) was added at r.t. and the reaction was continued at 60° C. for 14 h. After completion of the reaction was confirmed by TLC, the reaction mixture was diluted with cold water and extraction with CH₂Cl₂ was performed.

The combined organic layers were washed with sat. brine, dried over anhydrous sodium sulfate and evaporated under reduced pressure. The obtained crude residue was purified by column chromatography over silica gel (3-5% MeOH in CH₂Cl₂) to afford the envisaged product 2-fluoro-4-isobutyl-6-(3-isopropyl-4-(pyridazin-3-ylmethyl)piperazin-1-yl)benzonitrile (74 mg, 75% over 2 steps).

A mixture of 2-fluoro-4-isobutyl-6-(3-isopropyl-4-(pyridazin-3-ylmethyl)piperazin-1-yl)benzonitrile (50 mg, 0.13 mmol), NaN₃ (66 mg, 1.01 mmol) and Bu₃SnCl (0.28 mL, 1.01 mmol) in toluene (10 mL) was stirred at 140° C. for 14 h in a sealed tube. After the completion of the reaction was confirmed by TLC, the reaction mixture was concentrated in vacuo. The residue was dissolved in CH₂Cl₂ and washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The crude compound thus obtained was purified by silica chromatography (6-8% of MeOH in CH₂Cl₂), followed by trituration with diethyl ether to afford 3-[[4-[3-fluoro-5-isobutyl-2-(2H-tetrazol-5-yl)phenyl]-2-isopropyl-piperazin-1-yl]methyl]pyridazine as an off-white solid (4 mg, 7%).

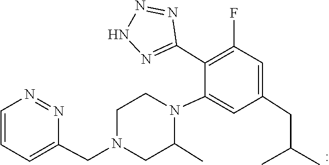 [SMILES: CC(C)Cc1cc(F)c(-c2nn[nH]n2)c(N2CCN(Cc3cccnn3)CC2C)c1]

Prepared through method E from intermediate 10. To a stirred solution of N-Boc protected 3-methylpiperazine (225 mg, 1.12 mmol) in DMF (15 mL) was added K₂CO₃ (424 mg, 3.06 mmol) at 0° C., after which the reaction was stirred at r.t. for 10 minutes. Then intermediate 10 (200 mg, 1.02 mmol) was added and the reaction was continued at 80° C. for 12 h. After completion of the reaction was confirmed by TLC, the reaction mixture was diluted with cold water and extracted with CH₂Cl₂. The combined organic layers were washed with sat. brine, dried over anhydrous sodium sulfate and evaporated under reduced pressure. The obtained crude residue was purified by column chromatography over silica gel (eluting with 30-40% EtOAc in hexane) to afford tert-butyl 4-(2-cyano-3-fluoro-5-isobutylphenyl)-3-methylpiperazine-1-carboxylate as a gummy liquid (127 mg, 33%).

To the stirred solution of tert-butyl 4-(2-cyano-3-fluoro-5-isobutylphenyl)-3-methylpiperazine-1-carboxylate (120 mg, 0.32 mmol) in 1,4-dioxane (5 mL), HCl (g) in dioxane (10 mL) was added at 0° C. and the reaction was continued at r.t. for 2 h. After the completion of the reaction was confirmed by TLC, the reaction mixture was evaporated to dryness under reduced pressure, then washed with hexane to afford a crude residue. The obtained crude compound was taken to the next step without purification (93 mg crude).

To a stirred solution of 2-fluoro-4-isobutyl-6-(2-methylpiperazin-1-yl)benzonitrile hydrochloride in DMF (10 mL) was added Et₃N (0.16 mL, 1.12 mmol) at 0° C., after which the reaction was stirred at r.t. for 10 minutes. Then 3-(chloromethyl)pyridazine (50 mg, 0.38 mmol) was added at r.t. and the reaction was continued at this temperature for 8 h. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The crude residue was purified by column chromatography over silica gel eluting with 2-5% MeOH in CH₂Cl₂ to afford the targeted product 2-fluoro-4-isobutyl-6-(2-methyl-4-(pyridazin-3-ylmethyl)piperazin-1-yl)benzonitrile (92 mg, 78% over 2 steps).

A mixture of this isolated nitrile (80 mg, 0.22 mmol), NaN₃ (113 mg, 1.74 mmol) and Bu₃SnCl (0.47 mL, 1.74 mmol) in toluene (8 mL) was stirred at 140° C. for 16 h in a sealed tube. After the completion of the reaction was confirmed by TLC, the reaction mixture was evaporated under reduced pressure. The residue was dissolved in CH₂Cl₂ and washed with a 10% NaOH solution. The aqueous layer was then neutralized with a citric acid solution and extracted with CH₂Cl₂. The combined organic layers were washed with sat. brine, dried over anhydrous sodium sulfate and concentrated in vacuo. The remaining crude compound was purified by silica chromatography (4-5% of MeOH in CH₂Cl₂) to afford 3-[[4-[3-fluoro-5-isobutyl-2-(2H-tetrazol-5-yl)phenyl]-3-methyl-piperazin-1-yl]methyl]pyridazine, which was additionally triturated with diethyl ether to obtain an off-white solid (6 mg, 7%).

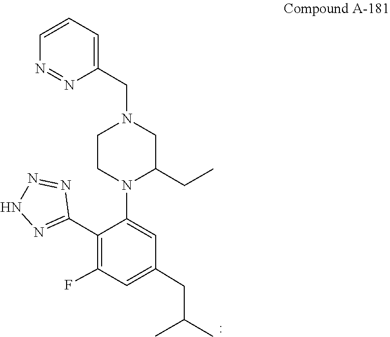 [SMILES: CCC1CN(Cc2cccnn2)CCN1c1cc(CC(C)C)cc(F)c1-c1nn[nH]n1]

Prepared through method F. To a stirred solution of tert-butyl-3-ethylpiperazine-1-carboxylate (296 mg, 1.38 mmol) in DMF (15 mL) was added K₂CO₃ (476 mg, 3.44 mmol) at 0° C., after which the reaction was stirred at r.t. for 10 minutes. Then 4-bromo-2,6-difluorobenzonitrile (250 mg, 1.15 mmol) was added and the reaction was continued at 80° C. for 12 h. After completion of the reaction was confirmed by TLC, the reaction mixture was diluted with cold water and extracted with CH₂Cl₂. The combined organic layers were washed with sat. brine, dried over anhydrous sodium sulfate and evaporated under reduced pressure. The obtained crude residue was purified by column chromatography over silica gel (eluting with 35-40% EtOAc in hexane) to afford tert-butyl 4-(5-bromo-2-cyano-3-fluorophenyl)-3-ethylpiperazine-1-carboxylate (222 mg, 47%).

To a stirred solution of the isolated SNAr product (220 mg, 0.53 mmol) in 1,4-dioxane was added 4,4,5,5-tetramethyl-2-(2-methylprop-1-en-1-yl)-1,3,2-dioxaborolane (117 mg, 0.64 mmol), followed by K₂CO₃ (184 mg, 1.33 mmol) and the resulting mixture was bubbled with argon for 20 min. Then Pd(dppf)Cl₂ (39 mg, 0.053 mmol) was added after which the reaction was heated to 80° C. for 6 h until completion of reaction was observed by TLC. The reaction mixture was concentrated under reduced pressure to remove the volatiles and the residue was re-dissolved with ethyl acetate and washed with water and sat. brine. Subsequent drying over anhydrous sodium sulfate and evaporation under reduced pressure, followed by column chromatography (SiO₂, 30-35% EtOAc in hexane) afforded the compound of interest as a gummy solid (172 mg, 83%).

A stirred solution of tert-butyl 4-(2-cyano-3-fluoro-5-(2-methylprop-1-en-1-yl)phenyl)-3-ethylpiperazine-1-carboxylate (170 mg, 0.44 mmol) in MeOH was hydrogenated over 10% Pd/C (0.017 g) under 5 Kg/cm² H₂ pressure using a Parr hydrogenator for 5 h at ambient temperature. After confirming the completion of the reaction by LC-MS, the reaction mixture was filtered through a Celite® bed and was evaporated under reduced pressure to afford the crude hydrogenated compound (143 mg crude). The obtained crude residue was taken to the next step without further purification.

To a stirred solution of tert-butyl 4-(2-cyano-3-fluoro-5-isobutylphenyl)-3-ethylpiperazine-1-carboxylate (140 mg, 0.36 mmol) in 1,4 dioxane (5 mL) was added HCl (g) in dioxane (10 mL) at 0° C. Upon completion of the addition, the reaction was allowed to slowly warm up to room temperature and kept stirring at r.t. for 3 h until complete conversion was obtained (via TLC). The reaction mixture was concentrated in vacuo, followed by washing of the crude residue with hexane. The crude compound was taken to the next step without additional purification (104 mg crude).

To a stirred solution of the deprotected compound (100 mg, 0.31 mmol) in DMF (10 mL) at 0° C. was added triethylamine (0.17 mL, 1.23 mmol) dropwise, after which the reaction was continued at room temperature for 10 minutes. Subsequently, 3-(chloromethyl)pyridazine (47 mg, 0.37 mmol) was added and the reaction was kept stirring at room temperature for 8 h. After completion of the reaction was confirmed by TLC, the reaction mixture was diluted with cold water and extracted with CH₂Cl₂. The combined organic layers were washed with water, sat. brine, dried over anhydrous sodium sulfate and evaporated under reduced pressure. The crude obtained was purified by column chromatography over silica gel (2-5% MeOH in CH₂Cl₂), affording 2-(2-ethyl-4-(pyridazin-3-ylmethyl)piperazin-1-yl)-6-fluoro-4-isobutylbenzonitrile as an off-white solid (102 mg, 87%).

In a final reaction, a mixture of the nitrile containing intermediate (100 mg, 0.26 mmol), NaN₃ (136 mg, 2.1 mmol) and Bu₃SnCl (0.57 mL, 2.1 mmol) in toluene (8 mL) was stirred at 140° C. for 16 h in a sealed tube. After the completion of the reaction was confirmed by TLC, an aq. work-up as described in method F was performed. The crude compound was purified by silica chromatography (5-6% of MeOH in CH₂Cl₂), followed by ultimate trituration with diethyl ether to afford 3-[[3-ethyl-4-[3-fluoro-5-isobutyl-2-(2H-tetrazol-5-yl)phenyl]piperazin-1-yl]methyl]pyridazine as an off-white solid (11 mg, 10%).

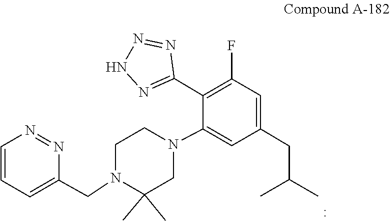 [SMILES: CC(C)Cc1cc(F)c(-c2nn[nH]n2)c(N2CCN(Cc3cccnn3)C(C)(C)C2)c1]

Prepared through method G from intermediate 10. To a stirred solution of tert-butyl 2,2-dimethylpiperazine-1-carboxylate (200 mg, 0.93 mmol) in DMF (10 mL) was added DIPEA (0.49 mL, 2.80 mmol) at 0° C., after which the reaction was stirred at r.t. for 10 minutes. Then 3-(chloromethyl)pyridazine (144 mg, 1.12 mmol) was added and the reaction was continued at 60° C. for 12 h. After completion of the reaction was confirmed by TLC, the reaction mixture was worked up as described in method G. Subsequent column chromatography over silica gel (eluting with 2-3% MeOH in CH₂Cl₂) afforded tert-butyl 2,2-dimethyl-4-(pyridazin-3-ylmethyl)piperazine-1-carboxylate (162 mg, 57%).

To a stirred solution of tert-butyl 2,2-dimethyl-4-(pyridazin-3-ylmethyl)piperazine-1-carboxylate (160 mg, 0.52 mmol) in 1,4-dioxane (5 mL) was added HCl (g) in dioxane (20 mL) at 0° C. and the reaction was continued at r.t. for 5 h. After the completion of the reaction was confirmed by TLC, the reaction mixture was evaporated to dryness under reduced pressure. Next, the residue was washed with hexane to afford a crude 3-((3,3-dimethylpiperazin-1-yl)methyl)pyridazine hydrochloride, which was used as such in the next step (123 mg crude).

To a stirred solution of 3-((3,3-dimethylpiperazin-1-yl)methyl)pyridazine hydrochloride (120 mg, 0.58 mmol) in DMF (10 mL) was added DIPEA (0.25 mL, 1.45 mmol) and K₂CO₃ (200 mg, 1.45 mmol) at 0° C. after which the reaction was stirred at r.t. for 10 minutes. Next, 2,6-difluoro-4-isobutylbenzonitrile (125 mg, 0.64 mmol) was added at r.t. and the reaction was continued at 60° C. for 12 h. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The obtained crude residue was purified by column chromatography over silica gel (4-5% MeOH in CH₂Cl₂) to afford the envisaged product 2-(2,2-dimethyl-4-(pyridazin-3-ylmethyl)piperazin-1-yl)-6-fluoro-4-isobutylbenzonitrile (83 mg, 42% over 2 steps).

A mixture of this isolated nitrile (80 mg, 0.21 mmol), NaN₃ (109 mg, 1.68 mmol) and Bu₃SnCl (0.46 mL, 1.68 mmol) in toluene (8 mL) was stirred at 140° C. for 14 h in a sealed tube. After the completion of the reaction was confirmed by TLC, the reaction mixture was concentrated in vacuo. The residue was dissolved in CH₂Cl₂ and washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The obtained crude compound was purified by silica chromatography (6-8% of MeOH in CH₂Cl₂), followed by trituration with diethyl ether, to afford 3-[[4-[3-fluoro-5-isobutyl-2-(2H-tetrazol-5-yl)phenyl]-2,2-dimethyl-piperazin-1-yl]methyl]pyridazine as an off-white solid (5 mg, 6%).

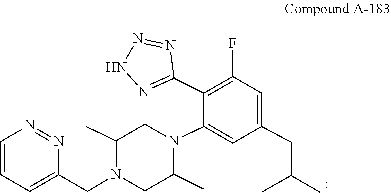 [SMILES: CC(C)Cc1cc(F)c(-c2nn[nH]n2)c(N2CC(C)N(Cc3cccnn3)CC2C)c1]

Prepared through method F. To a stirred solution of tert-butyl 2,5-dimethylpiperazine-1-carboxylate (236 mg, 1.10 mmol) in DMF (15 mL) was added DIPEA (0.40 mL, 2.29 mmol) at 0° C., after which the reaction was stirred at r.t. for 10 minutes. Then 4-bromo-2,6-difluorobenzonitrile (200 mg, 0.92 mmol) was added and the reaction was continued at 100° C. for 14 h. After completion of the reaction was confirmed by TLC, the reaction mixture was worked up as described in method F. Subsequent column chromatography over silica gel (eluting with 40-45% EtOAc in hexane) delivered tert-butyl 4-(5-bromo-2-cyano-3-fluorophenyl)-2,5-dimethylpiperazine-1-carboxylate (208 mg, 55%).

To a stirred solution of tert-butyl 4-(5-bromo-2-cyano-3-fluorophenyl)-2,5-dimethylpiperazine-1-carboxylate (200 mg, 0.49 mmol) in 1,4-dioxane was added 4,4,5,5-tetramethyl-2-(2-methylprop-1-en-1-yl)-1,3,2-dioxaborolane (106 mg, 0.58 mmol), followed by K₂CO₃ (168 mg, 1.21 mmol) and the resultant mixture was bubbled with argon for 20 min. Then Pd(dppf)Cl₂ (35 mg, 0.049 mmol) was added after which the reaction was heated to 80° C. for 10 h until completion of the reaction was observed by TLC. The reaction mixture was concentrated under reduced pressure to remove the volatiles and the residue was re-dissolved with ethyl acetate and washed with water and sat. brine. Subsequent drying over anhydrous sodium sulfate and evaporation under reduced pressure, followed by column chromatography (SiO₂, 30-40% EtOAc in hexane) afforded the compound of interest as a gummy solid (160 mg, 85%).

A stirred solution of tert-butyl 4-(2-cyano-3-fluoro-5-(2-methylprop-1-en-1-yl)phenyl)-2,5-dimethylpiperazine-1-carboxylate (160 mg, 0.41 mmol) in MeOH was hydrogenated over 10% Pd/C (0.016 g) under 5 Kg/cm² H₂ pressure using a Parr hydrogenator for 4 h at ambient temperature. After confirming the completion of reaction by LC-MS, the reaction mixture was filtered through a Celite® bed and was evaporated under reduced pressure to afford the crude hydrogenated compound (128 mg crude). The obtained crude residue was taken to the next step without further purification.

To the stirred solution of tert-butyl 4-(2-cyano-3-fluoro-5-isobutylphenyl)-2,5-dimethylpiperazine-1-carboxylate (125 mg, 0.32 mmol) in 1,4 dioxane (5 mL) was added HCl (g) in dioxane (10 mL) at 0° C. Upon completion of the addition, the reaction was allowed to slowly warm up to room temperature and kept stirring at r.t. for 3 h until complete conversion was obtained (via TLC). The reaction mixture was concentrated in vacuo, followed by washing of the crude residue with hexane. The crude compound was taken to the next step without additional purification (83 mg crude).

To a stirred solution of the hydrochloride salt (80 mg, 0.25 mmol) in DMF (10 mL) at 0° C. was added DIPEA (0.11 mL, 0.61 mmol) dropwise, followed by addition of Cs₂CO₃ (120 mg, 0.37 mmol), after which the reaction was continued at room temperature for 10 minutes. Subsequently, 3-(chloromethyl)pyridazine (38 mg, 0.29 mmol) was added and the reaction was kept stirring at 80° C. for 16 h. After completion of the reaction was confirmed by TLC, the reaction mixture was diluted with cold water and extracted with CH₂Cl₂. The combined organic layers were washed with water, sat. brine, dried over anhydrous sodium sulfate and evaporated under reduced pressure. The crude obtained was purified by column chromatography over silica gel (2-3% MeOH in CH₂Cl₂), affording 2-(2,5-dimethyl-4-(pyridazin-3-ylmethyl)piperazin-1-yl)-6-fluoro-4-isobutylbenzonitrile as an off-white solid (64 mg, 68%).

A mixture of the intermediate nitrile (60 mg, 0.16 mmol), NaN₃ (82 mg, 1.26 mmol) and Bu₃SnCl (0.34 mL, 1.26 mmol) in toluene (8 mL) was stirred at 140° C. for 20 h in a sealed tube. After the completion of the reaction was confirmed by TLC, an aq. work-up as described in method F was performed. The crude compound was purified by silica chromatography (5-6% of MeOH in CH₂Cl₂), followed by trituration with diethyl ether to afford the targeted tetrazole as a colorless gummy solid (5 mg, 7%).

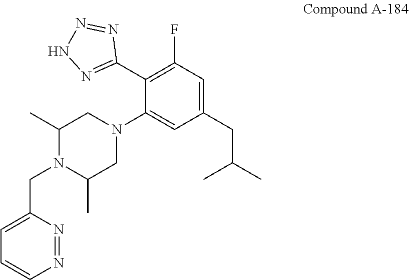 [SMILES: CC(C)Cc1cc(F)c(-c2nn[nH]n2)c(N2CC(C)N(Cc3cccnn3)C(C)C2)c1]

Prepared through method F. To a stirred solution of tert-butyl 2,6-dimethylpiperazine-1-carboxylate (236 mg, 1.10 mmol) in DMF (15 mL) was added DIPEA (0.40 mL, 2.29 mmol) at 0° C., after which the reaction was stirred at r.t. for 10 minutes. Then 4-bromo-2,6-difluorobenzonitrile (200 mg, 0.92 mmol) was added and the reaction was continued at 100° C. for 14 h. After completion of the reaction was confirmed by TLC, the reaction mixture was diluted with cold water and extracted with CH₂Cl₂. The combined organic layers were washed with sat. brine, dried over anhydrous sodium sulfate and evaporated under reduced pressure. The obtained crude residue was purified by column chromatography over silica gel (eluting with 35-40% EtOAc in hexane) to afford tert-butyl 4-(5-bromo-2-cyano-3-fluorophenyl)-2,6-dimethylpiperazine-1-carboxylate (212 mg, 56%).

To a stirred solution of tert-butyl 4-(5-bromo-2-cyano-3-fluorophenyl)-2,6-dimethylpiperazine-1-carboxylate (210 mg, 0.51 mmol) in 1,4-dioxane was added 4,4,5,5-tetramethyl-2-(2-methylprop-1-en-1-yl)-1,3,2-dioxaborolane (111 mg, 0.61 mmol), followed by K₂CO₃ (176 mg, 1.27 mmol) and the resultant mixture was bubbled with argon for 20 min. Then Pd(dppf)Cl₂ (37 mg, 0.051 mmol) was added after which the reaction was heated to 80° C. for 12 h until completion of the reaction was observed by TLC. The reaction mixture was concentrated under reduced pressure to remove the volatiles and the residue was re-dissolved with ethyl acetate and washed with water and sat. brine. Subsequent drying over anhydrous sodium sulfate and evaporation under reduced pressure, followed by column chromatography (SiO₂, 30-35% EtOAc in hexane) afforded the compound of interest as a gummy liquid (172 mg, 87%).

A stirred solution of tert-butyl 4-(2-cyano-3-fluoro-5-(2-methylprop-1-en-1-yl)phenyl)-2,6-dimethylpiperazine-1-carboxylate (165 mg, 0.43 mmol) in MeOH was hydrogenated over 10% Pd/C (0.017 g) under 5 Kg/cm² H₂ pressure using a Parr hydrogenator for 5 h at ambient temperature. After confirming the completion of reaction by LC-MS, the reaction mixture was filtered through a Celite® bed and was evaporated under reduced pressure to afford the crude hydrogenated compound (138 mg crude). The obtained crude residue was taken to the next step without further purification.

To the stirred solution of tert-butyl 4-(2-cyano-3-fluoro-5-isobutylphenyl)-2,6-dimethylpiperazine-1-carboxylate (135 mg, 0.35 mmol) in 1,4 dioxane (5 mL) was added HCl (g) in dioxane (10 mL) at 0° C. Upon completion of the addition, the reaction was allowed to slowly warm up to room temperature and kept stirring at r.t. for 3 h until complete conversion was obtained (via TLC). The reaction mixture was concentrated in vacuo, followed by washing of the crude residue with hexane. The crude compound was taken to the next step without additional purification (102 mg crude).

To a stirred solution of the deprotected compound (100 mg, 0.31 mmol) in DMF (10 mL) at 0° C. was added triethylamine (0.13 mL, 0.92 mmol) dropwise, after which the reaction was continued at room temperature for 10 minutes. Subsequently, 3-(chloromethyl)pyridazine (47 mg, 0.37 mmol) was added and the reaction was kept stirring at 80° C. for 12 h. After completion of the reaction was confirmed by TLC, the reaction mixture was diluted with cold water and extracted with CH₂Cl₂. The combined organic layers were washed with water, sat. brine, dried over anhydrous sodium sulfate and evaporated under reduced pressure. The crude obtained was purified by column chromatography over silica gel (2-5% MeOH in CH₂Cl₂), affording 2-(3,5-dimethyl-4-(pyridazin-3-ylmethyl)piperazin-1-yl)-6-fluoro-4-isobutylbenzonitrile as an off-white solid (82 mg, 70%) A mixture of the intermediate nitrile (80 mg, 0.21 mmol), NaN₃ (109 mg, 1.68 mmol) and Bu₃SnCl (0.46 mL, 1.68 mmol) in toluene (8 mL) was stirred at 150° C. for 16 h in a sealed tube. After the completion of the reaction was confirmed by TLC, an aq. work-up as described in method F was performed. The crude compound was purified by silica chromatography (5-6% of MeOH in CH₂Cl₂), followed by trituration with diethyl ether to afford the targeted tetrazole as an off-white solid (10 mg, 11%).

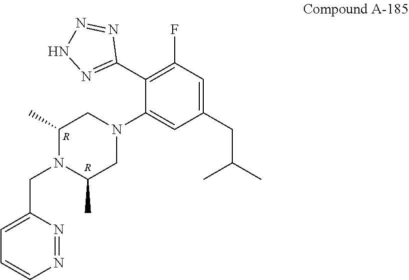

Prepared in a similar fashion as compound A-174 through method G by mixing tert-butyl (3R,5R)-3,5-dimethylpiperazine-1-carboxylate (120 mg, 0.56 mmol), DIPEA (0.293 mL, 1.68 mmol) and 3-(chloromethyl)pyridazine (86 mg, 0.672 mmol) at 60° C. for 14 h. After performing an aq. work-up, the organic residue was purified by silica chromatography (2-3% MeOH in CH₂Cl₂) to give tert-butyl (3R,5R)-3,5-dimethyl-4-(pyridazin-3-ylmethyl)piperazine-1-carboxylate (86 mg, 50%).

Subsequent Boc deprotection of tert-butyl (3R,5R)-3,5-dimethyl-4-(pyridazin-3-ylmethyl)piperazine-1-carboxylate (85 mg, 0.277 mmol) in 1,4-dioxane (1 mL) using HCl (g) in dioxane (2 mL) delivered the targeted hydrochloride salt after 5 h stirring at r.t.

To a stirred solution of 3-[[(2R,6R)-2,6-dimethylpiperazin-1-yl]methyl]pyridazine hydrochloride (65 mg, 0.268 mmol) in DMF (3 mL) were added DIPEA (0.117 mL, 0.669 mmol) and K₂CO₃ (93 mg, 0.669 mmol) at 0° C. after which the reaction was stirred at r.t. for 10 minutes. Next, 2,6-difluoro-4-isobutylbenzonitrile (57 mg, 0.295 mmol) was added at r.t. and the reaction was continued at 65° C. for 14 h. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The crude thus obtained was purified by column chromatography over silica gel eluting with 4-5% MeOH in CH₂Cl₂ to afford the envisaged 2-[(3R,5R)-3,5-dimethyl-4-(pyridazin-3-ylmethyl)piperazin-1-yl]-6-fluoro-4-isobutyl-benzonitrile (67 mg, 63% over 2 steps).

Final tetrazole reaction was performed, by mixing the nitrile (62 mg, 0.163 mmol), NaN₃ (85 mg, 1.30 mmol) and Bu₃SnCl (0.353 mL, 1.30 mmol) in toluene (5 mL) at 140° C. for 15 h in a sealed tube. Aq. work-up, followed by column chromatography (5-6% MeOH in CH₂Cl₂) and ultimate trituration of the purified compound with diethyl ether, yielded the envisaged tetrazole as an off-white solid (5 mg, 7%).

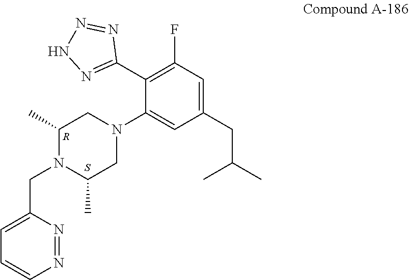

Prepared in a similar fashion as compound A-174 through method G by mixing tert-butyl (3R,5S)-3,5-dimethylpiperazine-1-carboxylate (120 mg, 0.56 mmol), DIPEA (0.293 mL, 1.68 mmol) and 3-(chloromethyl)pyridazine (86 mg, 0.672 mmol) at 60° C. for 14 h.

After performing an aq. work-up, the organic residue was purified by silica chromatography (2-3% MeOH in CH₂Cl₂) to give tert-butyl (3R,5S)-3,5-dimethyl-4-(pyridazin-3-ylmethyl)piperazine-1-carboxylate (95 mg, 55%).

Subsequent Boc deprotection of tert-butyl (3R,5S)-3,5-dimethyl-4-(pyridazin-3-ylmethyl)piperazine-1-carboxylate (95 mg, 0.310 mmol) in 1,4-dioxane (1 mL) using HCl (g) in dioxane (2 mL) delivered the targeted hydrochloride salt after 5 h stirring at r.t.

To a stirred solution of 3-[[(2S,6R)-2,6-dimethylpiperazin-1-yl]methyl]pyridazine hydrochloride (75 mg, 0.309 mmol) in DMF (3 mL) were added DIPEA (0.135 mL, 0.772 mmol) and K₂CO₃ (107 mg, 0.772 mmol) at 0° C. after which the reaction was stirred at r.t. for 10 minutes. Then, 2,6-difluoro-4-isobutylbenzonitrile (66 mg, 0.340 mmol) was added at r.t. and the reaction was continued at 65° C. for 14 h. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The crude thus obtained was purified by column chromatography over silica gel eluting with 4-5% MeOH in CH₂Cl₂ to afford the targeted substrate 2-[(3R,5S)-3,5-dimethyl-4-(pyridazin-3-ylmethyl)piperazin-1-yl]-6-fluoro-4-isobutyl-benzonitrile (70 mg, 59% over 2 steps).

Final tetrazole reaction was performed, by mixing the nitrile (65 mg, 0.170 mmol), NaN₃ (89 mg, 1.36 mmol) and Bu₃SnCl (0.370 mL, 1.36 mmol) in toluene (5 mL) at 140° C. for 15 h in a sealed tube. Aq. work-up, followed by column chromatography (6-8% MeOH in CH₂Cl₂) and ultimate trituration of the purified compound with diethyl ether, yielded the envisaged tetrazole as an off-white solid (7 mg, 9%).

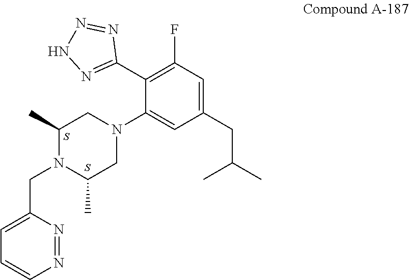

Prepared in a similar fashion as compound A-174 through method G by mixing tert-butyl (3S,5S)-3,5-dimethylpiperazine-1-carboxylate (120 mg, 0.56 mmol), DIPEA (0.293 mL, 1.68 mmol) and 3-(chloromethyl)pyridazine (86 mg, 0.672 mmol) at 60° C. for 14 h. After performing an aq. work-up, the organic residue was purified by silica chromatography (2-3% MeOH in CH₂Cl₂) to give tert-butyl (3S,5S)-3,5-dimethyl-4-(pyridazin-3-ylmethyl)piperazine-1-carboxylate (90 mg, 52%).

Subsequent Boc deprotection of tert-butyl (3S,5S)-3,5-dimethyl-4-(pyridazin-3-ylmethyl)piperazine-1-carboxylate (90 mg, 0.294 mmol) in 1,4-dioxane (1 mL) using HCl (g) in dioxane (2 mL) delivered the targeted hydrochloride salt after 5 h stirring at r.t.

To a stirred solution of 3-[[(2S,6S)-2,6-dimethylpiperazin-1-yl]methyl]pyridazine hydrochloride (70 mg, 0.288 mmol) in DMF (3 mL) were added DIPEA (0.126 mL, 0.721 mmol) and K₂CO₃ (100 mg, 0.721 mmol) at 0° C. after which the reaction was stirred at r.t. for 10 minutes. Next, 2,6-difluoro-4-isobutylbenzonitrile (62 mg, 0.317 mmol) was added at r.t. and the reaction was continued at 65° C. for 14 h. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The obtained crude residue was purified by column chromatography over silica gel eluting with 4-5% MeOH in CH₂Cl₂ to afford the desired 2-[(3S,5S)-3,5-dimethyl-4-(pyridazin-3-ylmethyl)piperazin-1-yl]-6-fluoro-4-isobutyl-benzonitrile (70 mg, 63% over 2 steps).

Final tetrazole reaction was performed, by mixing the nitrile (65 mg, 0.170 mmol), NaN₃ (89 mg, 1.36 mmol) and Bu₃SnCl (0.370 mL, 1.36 mmol) in toluene (5 mL) at 140° C. for 15 h in a sealed tube. Aq. work-up, followed by column chromatography (6-8% MeOH in CH₂Cl₂) and ultimate trituration of the purified compound with diethyl ether, yielded the envisaged tetrazole as an off-white solid (5 mg, 7%).

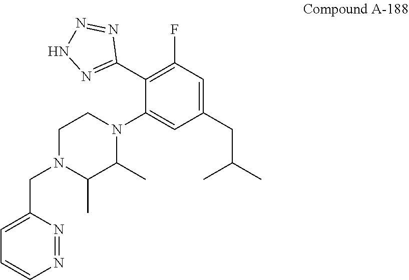 [SMILES: CC(C)Cc1cc(F)c(-c2nn[nH]n2)c(N2CCN(Cc3cccnn3)C(C)C2C)c1]

Prepared through method E from intermediate 10. To a stirred solution of 2,3-dimethylpiperazine (70 mg, 0.61 mmol) in DMSO (10 mL) was added DIPEA (0.27 mL, 1.53 mmol) at 0° C., after which the reaction was stirred at r.t. for 10 minutes. Next, intermediate 10 (100 mg, 0.51 mmol) was added and the reaction was continued at 100° C. for 12 h. After completion of the reaction was confirmed by TLC, an aq. work-up was performed as described in method E. The obtained crude residue was used as such in the next step (93 mg crude).

To a stirred solution of 2-(2,3-dimethylpiperazin-1-yl)-6-fluoro-4-isobutylbenzonitrile in DMF (10 mL) was added K₂CO₃ (141 mg, 1.02 mmol) at 0° C., after which the reaction was stirred at r.t. for 10 minutes. Then 3-(chloromethyl)pyridazine (79 mg, 0.61 mmol) and a catalytic amount of NaI were added at r.t. and the reaction was continued at this temperature for an additional 8 hours. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The crude thus obtained was purified by column chromatography over silica gel eluting with 2-3% MeOH in CH₂Cl₂ to afford the envisaged product 2-(2,3-dimethyl-4-(pyridazin-3-ylmethyl)piperazin-1-yl)-6-fluoro-4-isobutylbenzonitrile (134 mg, 69% over 2 steps).

A mixture of this isolated nitrile (80 mg, 0.21 mmol), NaN₃ (109 mg, 1.68 mmol) and Bu₃SnCl (0.46 mL, 1.68 mmol) in toluene (7 mL) was stirred at 150° C. for 18 h in a sealed tube. After the completion of the reaction was confirmed by TLC, the reaction mixture was evaporated under reduced pressure. The residue was re-dissolved in CH₂Cl₂ and washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The crude compound thus obtained was purified by silica chromatography (6-8% of MeOH in CH₂Cl₂) to afford the targeted substituted tetrazole which was additionally triturated with diethyl ether to obtain an off-white solid (11 mg, 12%).

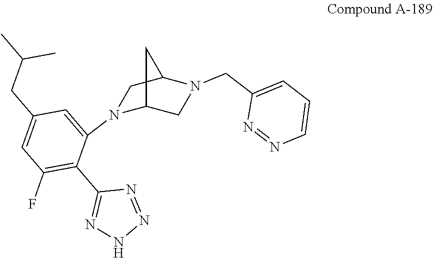 [SMILES: CC(C)Cc1cc(F)c(-c2nn[nH]n2)c(N2CC3CC2CN3Cc2cccnn2)c1]

Prepared through method E from intermediate 10. To a stirred solution of tert-butyl 2,5-diazabicyclo[2.2.1]heptane-2-carboxylate (305 mg, 1.53 mmol) in DMF (20 mL) was added K₂CO₃ (532 mg, 3.85 mmol) at 0° C., after which the reaction was stirred at r.t. for 10 minutes. Then, intermediate 10 (250 mg, 1.28 mmol) was added and the reaction was continued at 80° C. for 12 h. After completion of the reaction was confirmed by TLC, an aq. work-up was performed as described in method E. The obtained crude residue was purified by column chromatography over silica gel (eluting with 35-40% EtOAc in hexane) to yield tert-butyl 5-(2-cyano-3-fluoro-5-isobutylphenyl)-2,5-diazabicyclo[2.2.1]heptane-2-carboxylate as a gummy liquid (153 mg, 32%).

To a stirred solution of tert-butyl 5-(2-cyano-3-fluoro-5-isobutylphenyl)-2,5-diazabicyclo[2.2.1]heptane-2-carboxylate (150 mg, 0.40 mmol) in 1,4-dioxane (5 mL), HCl (g) in dioxane (15 mL) was added at 0° C. and the reaction was continued at r.t. for 2 h. After the completion of the reaction was confirmed by TLC, the reaction mixture was evaporated to dryness under reduced pressure, then washed with hexane to afford a crude residue. The obtained crude compound was used as such in the next step without purification (106 mg crude).

To a stirred solution of 2-(2,5-diazabicyclo[2.2.1]heptan-2-yl)-6-fluoro-4-isobutylbenzonitrile hydrochloride in DMF (10 mL) was added Et₃N (0.17 mL, 1.20 mmol) at 0° C., after which the reaction was stirred at r.t. for 10 minutes. Then 3-(chloromethyl)pyridazine (62 mg, 0.48 mmol) was added at r.t. and the reaction was continued at this temperature for 8 h. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The crude thus obtained was purified by column chromatography over silica gel eluting with 2-5% MeOH in CH₂Cl₂ to afford the targeted 2-fluoro-4-isobutyl-6-(5-(pyridazin-3-ylmethyl)-2,5-diazabicyclo[2.2.1]heptan-2-yl)benzonitrile (91 mg, 62% over 2 steps).

A mixture of this isolated nitrile (70 mg, 0.19 mmol), NaN₃ (100 mg, 1.53 mmol) and Bu₃SnCl (0.42 mL, 1.53 mmol) in toluene (7 mL) was stirred at 140° C. for 22 h in a sealed tube. After the completion of the reaction was confirmed by TLC, the reaction mixture was evaporated under reduced pressure. The residue was dissolved in CH₂Cl₂ and washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The crude compound thus obtained was purified via silica chromatography (6-8% of MeOH in CH₂Cl₂) to afford the desired substituted tetrazole which was additionally triturated with diethyl ether to obtain an off-white solid (8 mg, 10%).

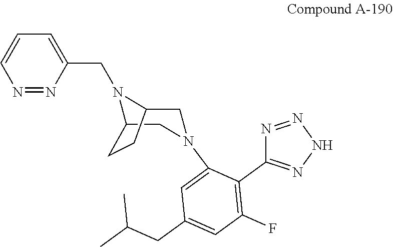 [SMILES: CC(C)Cc1cc(F)c(-c2nn[nH]n2)c(N2CC3CCC(C2)N3Cc2cccnn2)c1]

Prepared through method F. To a stirred solution of tert-butyl 3,8-diazabicyclo[3.2.1]octane-8-carboxylate (234 mg, 1.10 mmol) in DMF (15 mL) was added DIPEA (0.40 mL, 2.29 mmol) at 0° C., after which the reaction was stirred at r.t. for 10 minutes. Then 4-bromo-2,6-difluorobenzonitrile (200 mg, 0.92 mmol) was added and the reaction was continued at 80° C. for 12 h. After completion of the reaction was confirmed by TLC, the reaction mixture was worked up as described in method F. Subsequent column chromatography over silica gel (eluting with 40-45% EtOAc in hexane) delivered tert-butyl 3-(5-bromo-2-cyano-3-fluorophenyl)-3,8-diazabicyclo[3.2.1]octane-8-carboxylate (153 mg, 41%).

To a stirred solution of tert-butyl 3-(5-bromo-2-cyano-3-fluorophenyl)-3,8-diazabicyclo[3.2.1]octane-8-carboxylate (150 mg, 0.37 mmol) in 1,4-dioxane was added 4,4,5,5-tetramethyl-2-(2-methylprop-1-en-1-yl)-1,3,2-dioxaborolane (80 mg, 0.44 mmol), followed by K₂CO₃ (126 mg, 0.91 mmol) and the resultant mixture was bubbled with argon for 20 min. Then Pd(dppf)Cl₂ (14 mg, 0.037 mmol) was added after which the reaction was heated to 80° C. for 12 h until completion of reaction was observed by TLC. The reaction mixture was concentrated under reduced pressure to remove the volatiles and the residue was re-dissolved with ethyl acetate and washed with water and sat. brine. Subsequent drying over anhydrous sodium sulfate and evaporation under reduced pressure, followed by column chromatography (SiO₂, 30-40% EtOAc in hexane) afforded the compound of interest as a gummy solid (115 mg, 82%).

A stirred solution of tert-butyl 3-(2-cyano-3-fluoro-5-(2-methylprop-1-en-1-yl)phenyl)-3,8-diazabicyclo[3.2.1]octane-8-carboxylate (110 mg, 0.29 mmol) in MeOH was hydrogenated over 10% Pd/C (0.011 g) under 5 Kg/cm² H₂ pressure using a Parr hydrogenator for 4 h at ambient temperature. After confirming the completion of the reaction by LC-MS, the reaction mixture was filtered through a Celite® bed and was concentrated in vacuo to afford the crude hydrogenated compound (87 mg crude). The obtained crude residue was taken to the next step without further purification.

To a stirred solution of tert-butyl 3-(2-cyano-3-fluoro-5-isobutylphenyl)-3,8-diazabicyclo[3.2.1]octane-8-carboxylate (85 mg, 0.22 mmol) in 1,4 dioxane (5 mL) was added HCl (g) in dioxane (10 mL) at 0° C. Upon completion of the addition, the reaction was allowed to slowly warm up to room temperature and kept stirring at r.t. for 3 h until complete conversion was obtained (via TLC). The reaction mixture was concentrated in vacuo, followed by washing of the crude residue with hexane. The crude compound was taken to the next step without additional purification (53 mg crude).

To a stirred solution of 2-(3,8-diazabicyclo[3.2.1]octan-3-yl)-6-fluoro-4-isobutyl-benzonitrile hydrochloride (50 mg, 0.15 mmol) in DMF (10 mL) at 0° C. was added DIPEA (0.08 mL, 0.46 mmol) dropwise, after which the reaction was continued at room temperature for 10 minutes. Subsequently, 3-(chloromethyl)pyridazine (24 mg, 0.19 mmol) was added and the reaction was kept stirring at 60° C. for 12 h. After completion of the reaction was confirmed by TLC, the reaction mixture was diluted with cold water and extracted with CH₂Cl₂. The combined organic layers were washed with water, sat. brine, dried over anhydrous sodium sulfate and evaporated under reduced pressure. The crude obtained was purified by column chromatography over silica gel (2-4% MeOH in CH₂Cl₂), affording 2-fluoro-4-isobutyl-6-(8-(pyridazin-3-ylmethyl)-3,8-diazabicyclo[3.2.1]octan-3-yl)benzonitrile as an off-white solid (52 mg, 88%).

A mixture of the isolated intermediate nitrile (50 mg, 0.13 mmol), NaN₃ (69 mg, 1.05 mmol) and Bu₃SnCl (0.29 mL, 1.05 mmol) in toluene (5 mL) was stirred at 140° C. for 18 h in a sealed tube. After the completion of the reaction was confirmed by TLC, an aq. work-up as described in method F was performed. The crude compound was purified by silica chromatography (6-8% of MeOH in CH₂Cl₂), followed by trituration with diethyl ether to afford the targeted tetrazole as an off-white solid (8 mg, 14%).

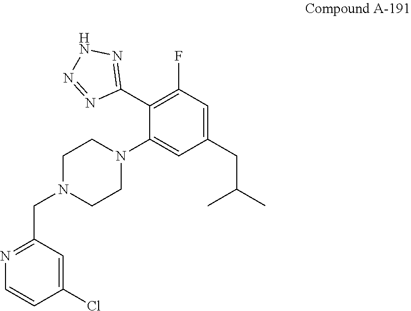 [SMILES: CC(C)Cc1cc(F)c(-c2nn[nH]n2)c(N2CCN(Cc3cc(Cl)ccn3)CC2)c1]

Prepared through method A from (4-chloro-2-pyridyl)methanol (150 mg, 1.045 mmol) and SOCl₂ (0.19 mL, 2.612 mmol) to give 4-chloro-2-(chloromethyl)pyridine as a gummy solid (147 mg, crude). By using intermediate 2 (150 mg, 0.574 mmol), DIPEA (0.20 mL, 1.149 mmol) and 4-chloro-2-(chloromethyl)pyridine (112 mg, 0.689 mmol) in a nucleophilic substitution reaction the desired compound was obtained after 15 h stirring at room temperature. After performing an aq. work-up, the organic residue was purified by silica chromatography (2-3% MeOH in CH₂Cl₂) to give 2-(4-((4-chloropyridin-2-yl)methyl)piperazin-1-yl)-6-fluoro-4-isobutylbenzonitrile as a gummy solid (155 mg, 75%).

Final tetrazole reaction was performed, by mixing the nitrile (140 mg, 0.362 mmol), NaN₃ (188 mg, 2.90 mmol) and Bu₃SnCl (0.78 mL, 2.90 mmol) in toluene (10 mL) at 140° C. for 14 h in a sealed tube. Aq. work-up followed by column chromatography and final trituration using diethyl ether, yielded the envisaged compound as a colorless solid (30 mg, 20%).

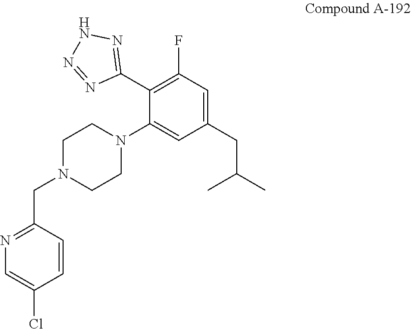 [SMILES: CC(C)Cc1cc(F)c(-c2nn[nH]n2)c(N2CCN(Cc3ccc(Cl)cn3)CC2)c1]

Prepared through method A from (5-chloro-2-pyridyl)methanol (150 mg, 1.045 mmol) and SOCl₂ (0.19 mL, 2.612 mmol) to give 5-chloro-2-(chloromethyl)pyridine as a gummy solid (127 mg, crude). To a solution of intermediate 2 (150 mg, 0.574 mmol) in DMF (10 mL) were added DIPEA (0.25 mL, 1.435 mmol) and 5-chloro-2-(chloromethyl)pyridine (112 mg, 0.689 mmol). The resulting reaction mixture was stirred at r.t. for 15 h. After performing an aq. work-up with EtOAc, the organic residue was purified by silica chromatography (2-3% MeOH in CH₂Cl₂) to give 2-(4-((5-chloropyridin-2-yl)methyl)piperazin-1-yl)-6-fluoro-4-isobutylbenzonitrile as a gummy solid (159 mg, 70%).

Final tetrazole reaction was performed, by mixing the nitrile (150 mg, 0.388 mmol), NaN₃ (202 mg, 3.11 mmol) and Bu₃SnCl (0.84 mL, 3.11 mmol) in toluene (10 mL) at 140° C. for 14 h in a sealed tube. Aq. work-up followed by column chromatography and final trituration with diethyl ether yielded the desired tetrazole as a colorless solid (30 mg, 21%).

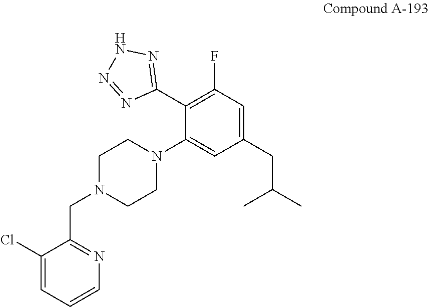 [SMILES: CC(C)Cc1cc(F)c(-c2nn[nH]n2)c(N2CCN(Cc3ncccc3Cl)CC2)c1]

Prepared through method A from (3-chloro-2-pyridyl)methanol (150 mg, 1.045 mmol) and SOCl₂ (0.19 mL, 2.612 mmol) to give 3-chloro-2-(chloromethyl)pyridine as a gummy solid (120 mg, crude). By using intermediate 2 (150 mg, 0.574 mmol), DIPEA (0.25 mL, 1.435 mmol) and 3-chloro-2-(chloromethyl)pyridine (112 mg, 0.689 mmol) in a nucleophilic substitution reaction the desired compound was obtained after 15 h stirring at r.t. After performing an aq. work-up, the organic residue was purified by silica chromatography (2-3% MeOH in CH₂Cl₂) to give 2-(4-((3-chloropyridin-2-yl)methyl)piperazin-1-yl)-6-fluoro-4-isobutylbenzonitrile as a gummy solid (131 mg, 58%).

Final tetrazole reaction was performed, by mixing the nitrile (128 mg, 0.331 mmol), NaN₃ (175 mg, 2.652 mmol) and Bu₃SnCl (0.71 mL, 2.652 mmol) in toluene (10 mL) at 140° C. for 14 h in a sealed tube. Aq. work-up, followed by column chromatography and ultimate trituration of the purified compound with diethyl ether, yielded the envisaged tetrazole as a colorless solid (24 mg, 16%).

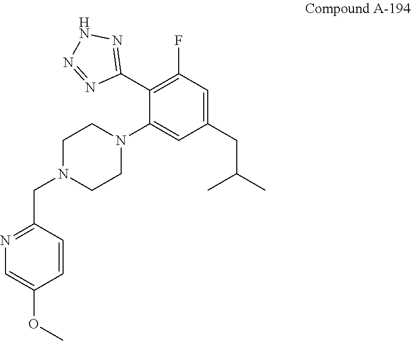 [SMILES: COc1ccc(CN2CCN(c3cc(CC(C)C)cc(F)c3-c3nn[nH]n3)CC2)nc1]

Prepared through method A from (5-methoxy-2-pyridyl)methanol (150 mg, 1.079 mmol) and SOCl₂ (0.20 mL, 2.697 mmol) to give 2-(chloromethyl)-5-methoxy-pyridine as a gummy solid (130 mg, crude). By using intermediate 2 (150 mg, 0.574 mmol), DIPEA (0.25 mL, 1.435 mmol) and 2-(chloromethyl)-5-methoxy-pyridine (108.6 mg, 0.689 mmol) in a nucleophilic substitution reaction, the desired compound was obtained after 15 h stirring at r.t. After performing an aq. work-up, the organic residue was purified by silica chromatography (2-3% MeOH in CH₂Cl₂) to give 2-fluoro-4-isobutyl-6-(4-((5-methoxypyridin-2-yl)methyl)piperazin-1-yl)benzonitrile as a gummy solid (142 mg, 65%).

Final tetrazole reaction was performed, by mixing the nitrile (135 mg, 0.353 mmol), NaN₃ (184 mg, 2.824 mmol) and Bu₃SnCl (0.76 mL, 2.824 mmol) in toluene (10 mL) at 140° C. for 14 h in a sealed tube. Aq. work-up, followed by column chromatography and ultimate trituration of the purified compound with diethyl ether yielded the desired 1-[3-fluoro-5-isobutyl-2-(2H-tetrazol-5-yl)phenyl]-4-[(5-methoxy-2-pyridyl)methyl]piperazine as a colorless solid (30 mg, 23%).

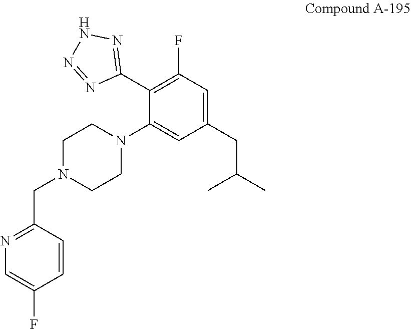 [SMILES: CC(C)Cc1cc(F)c(-c2nn[nH]n2)c(N2CCN(Cc3ccc(F)cn3)CC2)c1]

Prepared through method A from (5-fluoro-2-pyridyl)methanol (150 mg, 1.179 mmol) and SOCl₂ (0.22 mL, 2.949 mmol) to give 2-(chloromethyl)-5-fluoro-pyridine as a gummy solid (127 mg, crude). By mixing intermediate 2 (150 mg, 0.574 mmol), DIPEA (0.25 mL, 1.435 mmol) and 2-(chloromethyl)-5-fluoro-pyridine (100.4 mg, 0.689 mmol) in DMF (5 mL) the desired compound was obtained after 15 h stirring at r.t. After performing an aq. work-up, the organic residue was purified by silica chromatography (2-3% MeOH in CH₂Cl₂) to give 2-fluoro-6-(4-((5-fluoropyridin-2-yl)methyl)piperazin-1-yl)-4-isobutylbenzonitrile as a gummy solid (180 mg, 84%).

Final tetrazole reaction was performed, by mixing the nitrile (150 mg, 0.405 mmol), NaN₃ (210 mg, 3.243 mmol) and Bu₃SnCl (0.88 mL, 3.243 mmol) in toluene (10 mL) at 140° C. for 14 h in a sealed tube. Aq. work-up, followed by column chromatography and ultimate trituration of the purified compound with diethyl ether yielded the envisaged tetrazole as a colorless solid (30 mg, 18%).

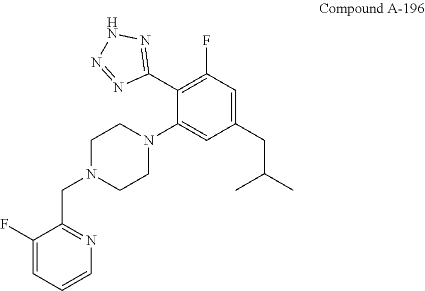 [SMILES: CC(C)Cc1cc(F)c(-c2nn[nH]n2)c(N2CCN(Cc3ncccc3F)CC2)c1]

Prepared through method A from (3-fluoro-2-pyridyl)methanol (150 mg, 1.179 mmol) and SOCl₂ (0.22 mL, 2.949 mmol) to give 2-(chloromethyl)-3-fluoro-pyridine as a gummy solid (127 mg, crude). By mixing intermediate 2 (150 mg, 0.574 mmol), DIPEA (0.25 mL, 1.435 mmol) and 2-(chloromethyl)-3-fluoro-pyridine (100.4 mg, 0.689 mmol) in DMF (5 mL) the desired compound was obtained after 15 h stirring at r.t. After performing an aq. work-up, the organic residue was purified by silica chromatography (2-3% MeOH in CH₂Cl₂) to give 2-fluoro-6-(4-((3-fluoropyridin-2-yl)methyl)piperazin-1-yl)-4-isobutylbenzonitrile as a gummy solid (159 mg, 70%).

Final tetrazole reaction was performed, by mixing the nitrile (150 mg, 0.405 mmol), NaN₃ (210 mg, 3.243 mmol) and Bu₃SnCl (0.88 mL, 3.243 mmol) in toluene (10 mL) at 140° C. for 14 h in a sealed tube. Aq. work-up, followed by column chromatography and ultimate trituration of the purified compound with diethyl ether afforded the desired tetrazole as a colorless solid (30 mg, 20%).

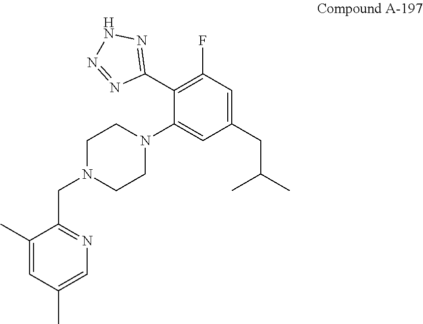 [SMILES: Cc1cnc(CN2CCN(c3cc(CC(C)C)cc(F)c3-c3nn[nH]n3)CC2)c(C)c1]

Prepared through method A from (3,5-dimethyl-2-pyridyl)methanol (150 mg, 1.093 mmol) and SOCl₂ (0.20 mL, 2.733 mmol) to give 2-(chloromethyl)-3,5-dimethyl-pyridine as a gummy solid (119 mg, crude). By mixing intermediate 2 (150 mg, 0.574 mmol), DIPEA (0.25 mL, 1.435 mmol) and 2-(chloromethyl)-3,5-dimethyl-pyridine (98 mg, 0.63 mmol) in DMF (5 mL) the desired compound was obtained after 15 h stirring at r.t. After performing an aq. work-up, the organic residue was purified by silica chromatography (2-3% MeOH in CH₂Cl₂) to give 2-(4-((3,5-dimethylpyridin-2-yl)methyl)piperazin-1-yl)-6-fluoro-4-isobutylbenzonitrile as a gummy solid (160 mg, 73%).

Final tetrazole reaction was performed, by mixing the nitrile (150 mg, 0.394 mmol), NaN₃ (205 mg, 3.157 mmol) and Bu₃SnCl (0.85 mL, 3.157 mmol) in toluene (10 mL) at 140° C. for 14 h in a sealed tube. Aq. work-up, followed by column chromatography and ultimate trituration of the purified compound with diethyl ether afforded 1-[(3,5-dimethyl-2-pyridyl)methyl]-4-[3-fluoro-5-isobutyl-2-(2H-tetrazol-5-yl)phenyl]piperazine as a colorless solid (30 mg, 17%).

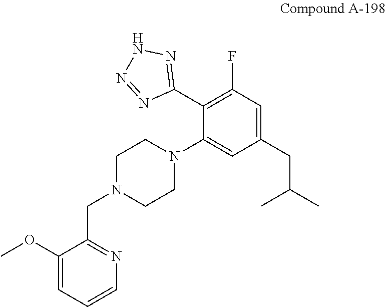 [SMILES: COc1cccnc1CN1CCN(c2cc(CC(C)C)cc(F)c2-c2nn[nH]n2)CC1]

Prepared through method A from (3-methoxy-2-pyridyl)methanol (150 mg, 1.079 mmol) and SOCl₂ (0.20 mL, 2.697 mmol) to give 2-(chloromethyl)-3-methoxy-pyridine as a gummy solid (121 mg, crude). By mixing intermediate 2 (150 mg, 0.574 mmol), DIPEA (0.25 mL, 1.435 mmol) and 2-(chloromethyl)-3-methoxy-pyridine (108.6 mg, 0.689 mmol), the desired compound was obtained after 15 h stirring at r.t. After performing an aq. work-up, the organic residue was purified by silica chromatography (2-3% MeOH in CH₂Cl₂) to give 2-fluoro-4-isobutyl-6-(4-((3-methoxypyridin-2-yl)methyl)piperazin-1-yl)benzonitrile as a gummy solid in good yield (180 mg, 84%).

Final tetrazole reaction was performed, by mixing the nitrile (170 mg, 0.445 mmol), NaN₃ (231 mg, 3.56 mmol) and Bu₃SnCl (0.96 mL, 3.56 mmol) in toluene (10 mL) at 140° C. for 14 h in a sealed tube. Aq. work-up, followed by column chromatography and ultimate trituration of the purified compound with diethyl ether yielded the envisaged final compound as a colorless solid (30 mg, 16%).

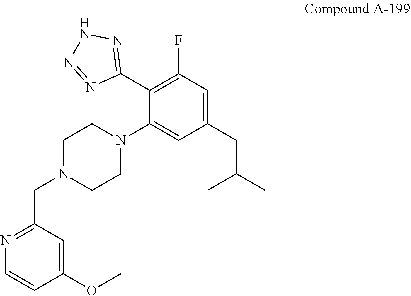 [SMILES: COc1ccnc(CN2CCN(c3cc(CC(C)C)cc(F)c3-c3nn[nH]n3)CC2)c1]

Prepared through method A from (4-methoxy-2-pyridyl)methanol (150 mg, 1.079 mmol) and SOCl₂ (0.20 mL, 2.697 mmol) to give 2-(chloromethyl)-4-methoxypyridine as a gummy solid (130 mg, crude). By using intermediate 2 (150 mg, 0.574 mmol), DIPEA (0.25 mL, 1.435 mmol) and 2-(chloromethyl)-4-methoxypyridine (109 mg, 0.69 mmol) in a nucleophilic substitution reaction the desired compound was obtained after 15 h stirring at r.t. After performing an aq. work-up, the organic residue was purified by silica chromatography (2-3% MeOH in CH₂Cl₂) to give 2-fluoro-4-isobutyl-6-(4-((4-methoxypyridin-2-yl)methyl)piperazin-1-yl)benzonitrile as a gummy solid (143 mg, 66%).

Final tetrazole reaction was performed, by mixing the nitrile (135 mg, 0.353 mmol), NaN₃ (184 mg, 2.824 mmol) and Bu₃SnCl (0.76 mL, 2.824 mmol) in toluene (10 mL) at 140° C. for 14 h in a sealed tube. Aq. work-up, followed by column chromatography and ultimate trituration of the purified compound with diethyl ether yielded the envisaged compound as a colorless solid (7 mg, 7%).

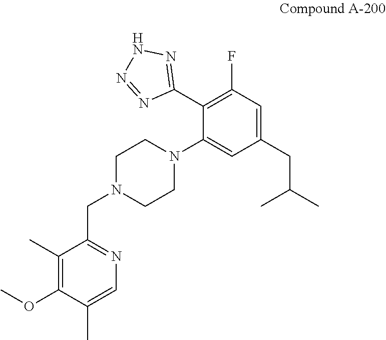 [SMILES: COc1c(C)cnc(CN2CCN(c3cc(CC(C)C)cc(F)c3-c3nn[nH]n3)CC2)c1C]

Prepared through method A from (4-methoxy-3,5-dimethyl-2-pyridyl)methanol (150 mg, 0.897 mmol) and SOCl₂ (0.16 mL, 2.243 mmol) to give 2-(chloromethyl)-4-methoxy-3,5-dimethyl-pyridine as a gummy solid (126 mg, crude). By mixing intermediate 2 (150 mg, 0.574 mmol), DIPEA (0.25 mL, 1.435 mmol) and 2-(chloromethyl)-4-methoxy-3,5-dimethyl-pyridine (117 mg, 0.63 mmol) in DMF (5 mL), the desired compound was obtained after 15 h stirring at r.t. After performing an aq. work-up, the organic residue was purified by silica chromatography (2-3% MeOH in CH₂Cl₂) to give 2-fluoro-4-isobutyl-6-(4-((4-methoxy-3,5-dimethylpyridin-2-yl)methyl)piperazin-1-yl)benzonitrile as a gummy solid (210 mg, 89%).

Final tetrazole reaction was performed, by mixing the nitrile (150 mg, 0.365 mmol), NaN₃ (190 mg, 2.92 mmol) and Bu₃SnCl (0.79 mL, 2.92 mmol) in toluene (10 mL) at 140° C. for 14 h in a sealed tube. Aq. work-up, followed by column chromatography and ultimate trituration of the purified compound with diethyl ether yielded the desired final compound as a colorless solid (10 mg, 7%).

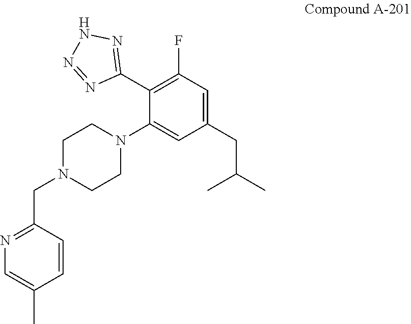 [SMILES: Cc1ccc(CN2CCN(c3cc(CC(C)C)cc(F)c3-c3nn[nH]n3)CC2)nc1]

Prepared through method A from (5-methyl-2-pyridyl)methanol (150 mg, 1.218 mmol) and SOCl₂ (0.22 mL, 3.045 mmol) to give 2-(chloromethyl)-5-methyl-pyridine as a gummy solid (127 mg, crude). By mixing intermediate 2 (150 mg, 0.574 mmol), DIPEA (0.25 mL, 1.435 mmol) and 2-(chloromethyl)-5-methyl-pyridine (102 mg, 0.63 mmol) in DMF (5 mL), the desired compound was obtained after 15 h stirring at r.t. After performing an aq. work-up, the organic residue was purified by silica chromatography (2-3% MeOH in CH₂Cl₂) to give 2-fluoro-4-isobutyl-6-(4-((5-methylpyridin-2-yl)methyl)piperazin-1-yl)benzonitrile as a gummy solid (175 mg, 75%).

Final tetrazole reaction was performed, by mixing the nitrile (170 mg, 0.464 mmol), NaN₃ (241 mg, 3.715 mmol) and Bu₃SnCl (1.0 mL, 3.715 mmol) in toluene (10 mL) at 140° C. for 14 h in a sealed tube. Aq. work-up, followed by column chromatography and ultimate trituration of the purified compound with diethyl ether yielded 1-[3-fluoro-5-isobutyl-2-(2H-tetrazol-5-yl)phenyl]-4-[(5-methyl-2-pyridyl)methyl]piperazine as a colorless solid (20 mg, 12%).

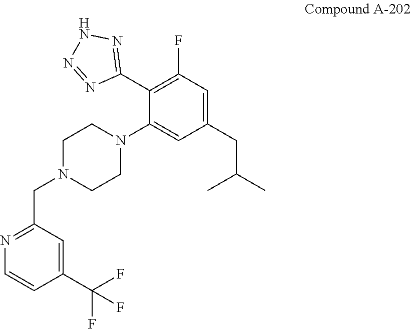 [SMILES: CC(C)Cc1cc(F)c(-c2nn[nH]n2)c(N2CCN(Cc3cc(C(F)(F)F)ccn3)CC2)c1]

Prepared through method A from [4-(trifluoromethyl)-2-pyridyl]methanol (150 mg, 0.846 mmol) and SOCl₂ (0.15 mL, 2.117 mmol) to give 2-(chloromethyl)-4-(trifluoromethyl)pyridine as a gummy solid (123 mg, crude). By mixing intermediate 2 (150 mg, 0.574 mmol), DIPEA (0.25 mL, 1.435 mmol) and 2-(chloromethyl)-4-(trifluoromethyl)pyridine (123 mg, 0.63 mmol) in DMF (5 mL), the desired compound was obtained after 15 h stirring at r.t. After performing an aq. work-up, the organic residue was purified by silica chromatography (2-3% MeOH in CH₂Cl₂) to give 2-fluoro-4-isobutyl-6-(4-((4-(trifluoromethyl)pyridin-2-yl)methyl)piperazin-1-yl)benzonitrile as a gummy solid (180 mg, 86%).

Final tetrazole reaction was performed, by mixing the nitrile (170 mg, 0.404 mmol), NaN₃ (210 mg, 3.23 mmol) and Bu₃SnCl (0.82 mL, 3.23 mmol) in toluene (10 mL) at 140° C. for 14 h in a sealed tube. Aq. work-up, followed by column chromatography and ultimate trituration of the purified compound with diethyl ether yielded 1-[3-fluoro-5-isobutyl-2-(2H-tetrazol-5-yl)phenyl]-4-[[4-(trifluoromethyl)-2-pyridyl]methyl]piperazine as a colorless solid (25 mg, 13%).

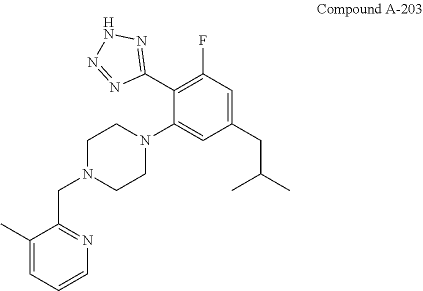 [SMILES: Cc1cccnc1CN1CCN(c2cc(CC(C)C)cc(F)c2-c2nn[nH]n2)CC1]

Prepared through method A from (3-methyl-2-pyridyl)methanol (150 mg, 1.218 mmol) and SOCl₂ (0.22 mL, 3.045 mmol) to give 2-(chloromethyl)-3-methylpyridine as a gummy solid (127 mg, crude). By mixing intermediate 2 (150 mg, 0.574 mmol), DIPEA (0.25 mL, 1.435 mmol) and 2-(chloromethyl)-3-methylpyridine (102 mg, 0.63 mmol) in DMF (5 mL) to perform a nucleophilic substitution reaction, the desired compound was obtained after 15 h stirring at r.t. After performing an aq. work-up, the organic residue was purified by silica chromatography (2-3% MeOH in CH₂Cl₂) to give 2-fluoro-4-isobutyl-6-(4-((3-methylpyridin-2-yl)methyl)piperazin-1-yl)benzonitrile as a gummy solid (159 mg, 70%).

Final tetrazole reaction was performed, by mixing the nitrile (100 mg, 0.273 mmol), NaN₃ (142 mg, 2.185 mmol) and Bu₃SnCl (0.59 mL, 2.185 mmol) in toluene (10 mL) at 140 5° C. for 14 h in a sealed tube. Aq. work-up, followed by column chromatography and ultimate trituration of the purified compound with diethyl ether yielded the expected compound as a colorless solid (18 mg, 16%).

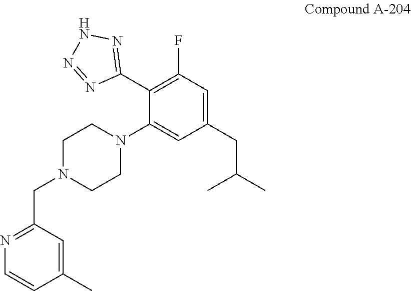 [SMILES: Cc1ccnc(CN2CCN(c3cc(CC(C)C)cc(F)c3-c3nn[nH]n3)CC2)c1]

Prepared through method A from (4-methyl-2-pyridyl)methanol (150 mg, 1.218 mmol) and SOCl₂ (0.22 mL, 3.045 mmol) to give 2-(chloromethyl)-4-methylpyridine as a gummy solid (127 mg, crude). By using intermediate 2 (150 mg, 0.574 mmol), DIPEA (0.25 mL, 1.435 mmol) and 2-(chloromethyl)-4-methylpyridine (102 mg, 0.63 mmol) in a nucleophilic substitution reaction, the desired compound was obtained after 15 h stirring at r.t. After performing an aq. work-up, the organic residue was purified by silica chromatography (2-3% MeOH in CH₂Cl₂) to give 2-fluoro-4-isobutyl-6-(4-((4-methylpyridin-2-yl)methyl)piperazin-1-yl)benzonitrile as a gummy solid (155 mg, 74%).

Final tetrazole reaction was performed, by mixing the nitrile (150 mg, 0.409 mmol), NaN₃ (213 mg, 3.278 mmol) and Bu₃SnCl (0.88 mL, 3.278 mmol) in toluene (10 mL) at 140° C. for 14 h in a sealed tube. Aq. work-up, followed by column chromatography and ultimate trituration of the purified compound with diethyl ether yielded the desired tetrazole as a colorless solid (20 mg, 16%).

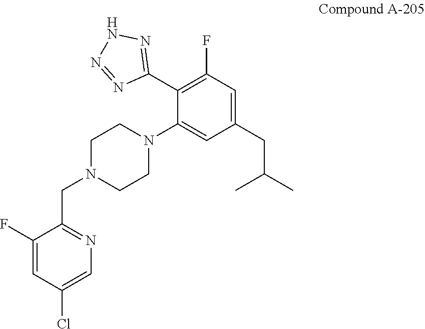 [SMILES: CC(C)Cc1cc(F)c(-c2nn[nH]n2)c(N2CCN(Cc3ncc(Cl)cc3F)CC2)c1]

Prepared through method A from (5-chloro-3-fluoro-2-pyridyl)methanol (150 mg, 0.928 mmol) and SOCl₂ (0.17 mL, 2.321 mmol) to give 5-chloro-2-(chloromethyl)-3-fluoropyridine as a gummy solid (125 mg, crude). By using intermediate 2 (150 mg, 0.574 mmol), DIPEA (0.25 mL, 1.435 mmol) and 5-chloro-2-(chloromethyl)-3-fluoropyridine (113 mg, 0.63 mmol) in a nucleophilic substitution reaction, the desired compound was obtained after 15 h stirring at r.t. After performing an aq. work-up, the organic residue was purified by silica chromatography (2-3% MeOH in CH₂Cl₂) to give 2-(4-((5-chloro-3-fluoropyridin-2-yl)methyl)piperazin-1-yl)-6-fluoro-4-isobutylbenzonitrile as a gummy solid (180 mg, 78%).

Final tetrazole reaction, by mixing the nitrile (150 mg, 0.371 mmol), NaN₃ (193 mg, 2.97 mmol) and Bu₃SnCl (0.8 mL, 2.97 mmol) in toluene (10 mL) at 140° C. for 14 h in a sealed tube was performed. Aq. work-up, followed by column chromatography and ultimate trituration of the purified compound with diethyl ether, yielded 1-[(5-chloro-3-fluoro-2-pyridyl)methyl]-4-[3-fluoro-5-isobutyl-2-(2H-tetrazol-5-yl)phenyl]piperazine as a colorless solid (25 mg, 15%).

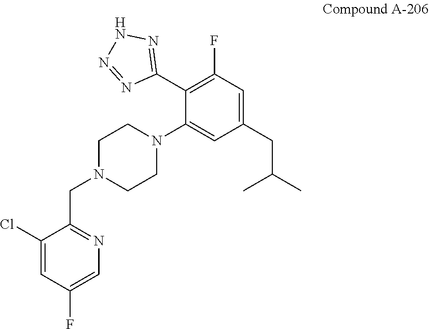 [SMILES: CC(C)Cc1cc(F)c(-c2nn[nH]n2)c(N2CCN(Cc3ncc(F)cc3Cl)CC2)c1]

Prepared through method A from (3-chloro-5-fluoro-2-pyridyl)methanol (150 mg, 0.928 mmol) and SOCl₂ (0.17 mL, 2.321 mmol) to give 3-chloro-2-(chloromethyl)-5-fluoro-pyridine as a gummy solid (132 mg, crude). By mixing intermediate 2 (150 mg, 0.574 mmol), DIPEA (0.25 mL, 1.435 mmol) and 3-chloro-2-(chloromethyl)-5-fluoro-pyridine (113 mg, 0.63 mmol) in DMF (5 mL), the desired compound was obtained after 15 h stirring at r.t. After performing an aq. work-up, the organic residue was purified by silica chromatography (2-3% MeOH in CH₂Cl₂) to give 2-(4-((3-chloro-5-fluoropyridin-2-yl)methyl)piperazin-1-yl)-6-fluoro-4-isobutylbenzonitrile as a gummy solid (130 mg, 56%).

Final tetrazole reaction was performed, by mixing the nitrile (125 mg, 0.322 mmol), NaN₃ (167 mg, 2.57 mmol) and Bu₃SnCl (0.7 mL, 2.57 mmol) in toluene (10 mL) at 140° C. for 14 h in a sealed tube. Aq. work-up, followed by column chromatography and ultimate trituration of the purified compound with diethyl ether yielded the desired final compound as a colorless solid (15 mg, 10%).

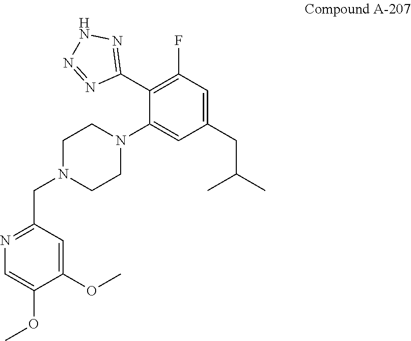 [SMILES: COc1cnc(CN2CCN(c3cc(CC(C)C)cc(F)c3-c3nn[nH]n3)CC2)cc1OC]

Prepared through method A from (4,5-dimethoxy-2-pyridyl)methanol (150 mg, 0.887 mmol) and SOCl₂ (0.16 mL, 2.217 mmol) to give 2-(chloromethyl)-4,5-dimethoxy-pyridine as a gummy solid (129 mg, crude). By mixing intermediate 2 (150 mg, 0.574 mmol), DIPEA (0.25 mL, 1.435 mmol) and 2-(chloromethyl)-4,5-dimethoxy-pyridine (118 mg, 0.63 mmol) in DMF (5 mL), the desired compound was obtained after 15 h stirring at r.t. After performing an aq. work-up, the organic residue was purified by silica chromatography (2-3% MeOH in CH₂Cl₂) to give 2-(4-((4,5-dimethoxypyridin-2-yl)methyl)piperazin-1-yl)-6-fluoro-4-isobutylbenzonitrile as a gummy solid (160 mg, 67%).

Final tetrazole reaction, by mixing the nitrile (150 mg, 0.364 mmol), NaN₃ (185 mg, 2.91 mmol) and Bu₃SnCl (0.78 mL, 2.91 mmol) in toluene (10 mL) at 140° C. for 14 h in a sealed tube was performed. Aq. work-up, followed by column chromatography and ultimate trituration of the purified compound with diethyl ether afforded 1-[(4,5-dimethoxy-2-pyridyl)methyl]-4-[3-fluoro-5-isobutyl-2-(2H-tetrazol-5-yl)phenyl]piperazine as a colorless solid (20 mg, 12%).

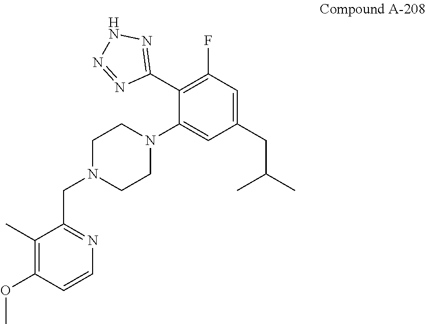 [SMILES: COc1ccnc(CN2CCN(c3cc(CC(C)C)cc(F)c3-c3nn[nH]n3)CC2)c1C]

Prepared through method A from (4-methoxy-3-methyl-2-pyridyl)methanol (150 mg, 0.979 mmol) and SOCl₂ (0.18 mL, 2.448 mmol) to give 2-(chloromethyl)-4-methoxy-3-methyl-pyridine as a gummy solid (121 mg, crude). By mixing intermediate 2 (150 mg, 0.574 mmol), DIPEA (0.25 mL, 1.435 mmol) and 2-(chloromethyl)-4-methoxy-3-methyl-pyridine (108 mg, 0.63 mmol) in DMF (5 mL), the desired compound was obtained after 15 h stirring at r.t. After performing an aq. work-up, the organic residue was purified by silica chromatography (2-3% MeOH in CH₂Cl₂) to give 2-fluoro-4-isobutyl-6-(4-((4-methoxy-3-methylpyridin-2-yl)methyl)piperazin-1-yl)benzonitrile as a gummy solid (132 mg, 58%).

Final tetrazole reaction was performed, by mixing the nitrile (130 mg, 0.328 mmol), NaN₃ (170 mg, 2.62 mmol) and Bu₃SnCl (0.7 mL, 2.62 mmol) in toluene (10 mL) at 140° C. for 14 h in a sealed tube. Aq. work-up, followed by column chromatography and ultimate trituration of the purified compound with diethyl ether yielded the desired final compound as a colorless solid (11 mg, 8%).

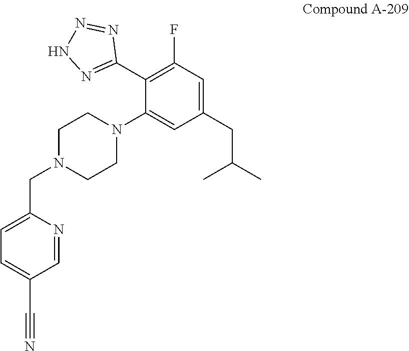 [SMILES: CC(C)Cc1cc(F)c(-c2nn[nH]n2)c(N2CCN(Cc3ccc(C#N)cn3)CC2)c1]

Prepared using method X by mixing intermediate 2 (400 mg, 1.532 mmol), NaN₃ (796 mg, 12.26 mmol) and Bu₃SnCl (3.32 mL, 12.26 mmol) in toluene (20 mL) at 140° C. for 14 h in a sealed tube. Aq. work-up, as described in method X, followed by column chromatography (SiO₂, 4-5% MeOH in CH₂Cl₂), yielded 1-(3-fluoro-5-isobutyl-2-(2H-tetrazol-5-yl)phenyl)piperazine as a colorless solid in moderate yield (200 mg, 44%).

Treating 6-(hydroxymethyl)pyridine-3-carbonitrile (150 mg, 1.12 mmol) with SOCl₂ (0.20 mL, 2.80 mmol) afforded 6-(chloromethyl)pyridine-3-carbonitrile as a gummy solid (116 mg, crude). To a stirred solution of the intermediate tetrazole (100 mg, 0.329 mmol) in DMF (10 mL) were added DIPEA (0.14 mL, 0.821 mmol) and 6-(chloromethyl)pyridine-3-carbonitrile (60 mg, 0.394 mmol). The resulting reaction mixture was stirred at r.t. for 15 h. Aq. work-up with EtOAc as described in method X and column chromatography (2-3% MeOH in CH₂Cl₂) yielded the expected compound as an off-white solid (11 mg, 10%).

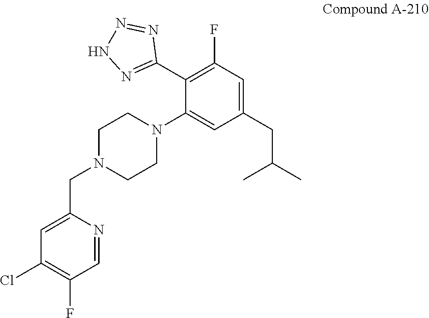 [SMILES: CC(C)Cc1cc(F)c(-c2nn[nH]n2)c(N2CCN(Cc3cc(Cl)c(F)cn3)CC2)c1]

Prepared through method A from (4-chloro-5-fluoro-2-pyridyl)methanol (150 mg, 0.928 mmol) and SOCl₂ (0.17 mL, 2.321 mmol) to give 4-chloro-2-(chloromethyl)-5-fluoro-pyridine as a gummy solid (128 mg, crude). By mixing intermediate 2 (150 mg, 0.574 mmol), DIPEA (0.25 mL, 1.435 mmol) and 4-chloro-2-(chloromethyl)-5-fluoro-pyridine (113 mg, 0.63 mmol) in DMF (5 mL), the desired compound was obtained after 15 h stirring at r.t. After performing an aq. work-up, the organic residue was purified by silica chromatography (2-3% MeOH in CH₂Cl₂) to give 2-(4-((4-chloro-5-fluoropyridin-2-yl)methyl)piperazin-1-yl)-6-fluoro-4-isobutylbenzonitrile as a gummy solid (200 mg, 87%).

Final tetrazole reaction was performed, by mixing the nitrile (180 mg, 0.445 mmol), NaN₃ (231 mg, 3.56 mmol) and Bu₃SnCl (0.96 mL, 3.56 mmol) in toluene (10 mL) at 140° C. for 14 h in a sealed tube. Aq. work-up, followed by column chromatography and ultimate trituration of the purified compound with diethyl ether yielded the expected tetrazole as a colorless solid (20 mg, 10%).

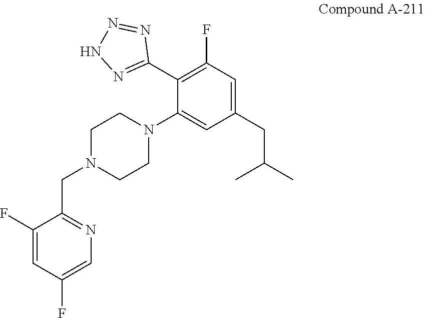 [SMILES: CC(C)Cc1cc(F)c(-c2nn[nH]n2)c(N2CCN(Cc3ncc(F)cc3F)CC2)c1]

Prepared through method A from (3,5-difluoro-2-pyridyl)methanol (150 mg, 1.034 mmol) and SOCl₂ (0.19 mL, 2.584 mmol) to give 2-(chloromethyl)-3,5-difluoro-pyridine as a gummy solid (132 mg, crude). By mixing intermediate 2 (150 mg, 0.574 mmol), DIPEA (0.25 mL, 1.435 mmol) and 2-(chloromethyl)-3,5-difluoro-pyridine (103 mg, 0.63 mmol) in DMF (5 mL), the desired compound was obtained after 15 h stirring at r.t. After performing an aq. work-up, the organic residue was purified by silica chromatography (2-3% MeOH in CH₂Cl₂) to give 2-(4-((3,5-difluoropyridin-2-yl)methyl)piperazin-1-yl)-6-fluoro-4-isobutylbenzonitrile as a gummy solid (159 mg, 70%).

Final tetrazole reaction, by mixing the nitrile (150 mg, 0.386 mmol), NaN₃ (201 mg, 3.09 mmol) and Bu₃SnCl (0.84 mL, 3.09 mmol) in toluene (10 mL) at 140° C. for 14 h in a sealed tube was performed. Aq. work-up, followed by column chromatography and ultimate trituration of the purified compound with diethyl ether yielded 1-[(3,5-difluoro-2-pyridyl)methyl]-4-[3-fluoro-5-isobutyl-2-(2H-tetrazol-5-yl)phenyl]piperazine as a colorless solid (30 mg, 20%).

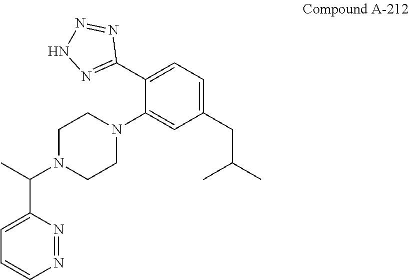 [SMILES: CC(C)Cc1ccc(-c2nn[nH]n2)c(N2CCN(C(C)c3cccnn3)CC2)c1]

Prepared using method U from 1-(pyridazin-3-yl)ethanol (360 mg, 2.90 mmol) and SOCl₂ (0.42 mL, 5.80 mmol) to give 3-(1-chloroethyl)pyridazine as a gummy solid (390 mg, crude). To a stirred solution of intermediate 1 (300 mg, 1.07 mmol) in DMF (10 mL) at 0° C. was added triethylamine (0.45 mL, 3.21 mmol) after which the reaction was stirred at r.t. for 10 minutes. Then K₂CO₃ (370 mg, 2.68 mmol) and NaI (catalytic amount), followed by 3-(1-chloroethyl)pyridazine (374 mg, 1.07 mmol), were added and the reaction was continued at r.t. for an additional 10 hours. After completion of the reaction was confirmed by TLC, the reaction mixture was diluted with cold water and extracted with EtOAc. The combined organic layers were washed with sat. brine, dried over anhydrous sodium sulfate and evaporated under reduced pressure. The crude residue was purified by silica chromatography, using 3-4% MeOH in CH₂Cl₂, to afford 4-isobutyl-2-(4-(1-(pyridazin-3-yl)ethyl)piperazin-1-yl)benzonitrile (205 mg, 64%).

A mixture of 4-isobutyl-2-(4-(1-(pyridazin-3-yl)ethyl)piperazin-1-yl)benzonitrile (200 mg, 0.25 mmol), NaN₃ (298 mg, 4.58 mmol) and Bu₃SnCl (1.14 mL, 4.58 mmol) in toluene (15 mL) was stirred at 140° C. for 20 h in a sealed tube. After the completion of the reaction was confirmed by TLC, the reaction mixture was evaporated in vacuo and the obtained residue re-dissolved in CH₂Cl₂. The organic phase was washed with a 10% NaOH solution. The aqueous layer was then neutralized with a citric acid solution, followed by extraction with CH₂Cl₂. After aq. work-up with sat. brine and drying over anhydrous sodium sulfate, the crude compound was purified by column chromatography over silica gel (6-7% MeOH in CH₂Cl₂) to afford compound A-212 as an off-white solid (15 mg, 7%).

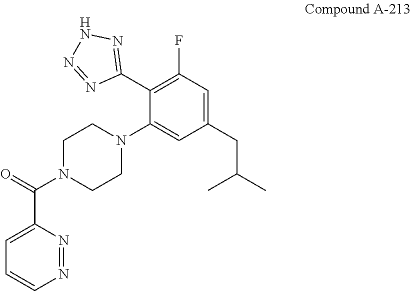 [SMILES: CC(C)Cc1cc(F)c(-c2nn[nH]n2)c(N2CCN(C(=O)c3cccnn3)CC2)c1]

A solution of intermediate 2 (300 mg, 1.148 mmol), NaN₃ (597 mg, 9.183 mmol) and Bu₃SnCl (2.49 mL, 9.183 mmol) in toluene (30 mL) was stirred at 140° C. for 14 h in a sealed tube. After the completion of the reaction was confirmed by TLC, the reaction mixture was evaporated under reduced pressure. The obtained residue was re-dissolved in CH₂Cl₂ and washed with a 10% NaOH solution. Next, the aqueous layer was neutralized with a citric acid solution and extracted with CH₂Cl₂. The combined organic layers were washed with sat. brine, dried over anhydrous sodium sulfate and concentrated in vacuo. Column chromatography (SiO₂, 2-3% of MeOH in CH₂Cl₂), followed by trituration in diethyl ether, afforded 1-(3-fluoro-5-isobutyl-2-(2H-tetrazol-5-yl)phenyl)piperazine as a colorless solid (150 mg, 43%).

To a stirred solution of 1-[3-fluoro-5-isobutyl-2-(2H-tetrazol-5-yl)phenyl]piperazine (100 mg, 0.329 mmol) in DMF (5 mL) was added pyridazine-3-carboxylic acid (43 mg, 0.345 mmol), followed by DIPEA (0.143 mL, 0.821 mmol) and HATU (150 mg, 0.394 mmol). Upon completion of the addition, the reaction mixture was allowed to stir at r.t. for 14 h until complete conversion was observed by TLC. The reaction mixture was diluted with water and extraction with EtOAc was performed. The combined organic layers were washed with sat. brine, dried over anhydrous sodium sulfate and evaporated under reduced pressure. The crude residue was purified by silica chromatography (4-5% of MeOH in CH₂Cl₂), followed by ultimate trituration in diethyl ether to afford [4-[3-fluoro-5-isobutyl-2-(2H-tetrazol-5-yl)phenyl]piperazin-1-yl]-pyridazin-3-yl-methanone as a colorless solid (18 mg, 13%).

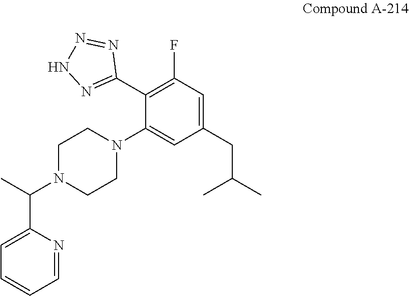 [SMILES: CC(C)Cc1cc(F)c(-c2nn[nH]n2)c(N2CCN(C(C)c3ccccn3)CC2)c1]

Prepared through method A from 1-(2-pyridyl)ethanol (250 mg, 2.03 mmol) and SOCl₂ (0.37 mL, 5.08 mmol) to give 2-(1-chloroethyl)pyridine as a gummy solid (201 mg, crude). By using intermediate 2 (250 mg, 0.957 mmol), DIPEA (0.42 mL, 2.392 mmol) and 2-(1-chloroethyl)pyridine (163 mg, 1.148 mmol) in a nucleophilic substitution reaction the desired compound was obtained after 12 h stirring at 60° C. After performing an aq. work-up, the organic residue was purified by silica chromatography (2-3% MeOH in CH₂Cl₂) to give 2-fluoro-4-isobutyl-6-[4-[1-(2-pyridyl)ethyl]piperazin-1-yl]benzonitrile as a gummy solid (200 mg, 57%).

Final tetrazole reaction was performed, by mixing the nitrile (200 mg, 0.546 mmol), NaN₃ (284 mg, 4.37 mmol) and Bu₃SnCl (1.18 mL, 4.37 mmol) in toluene (10 mL) at 140° C. for 14 h in a sealed tube. Aq. work-up, followed by column chromatography (SiO₂, 4-5% MeOH in CH₂Cl₂) and ultimate trituration of the purified compound with diethyl ether yielded the desired 1-[3-fluoro-5-isobutyl-2-(2H-tetrazol-5-yl)phenyl]-4-[1-(2-pyridyl)ethyl]piperazine as a colorless solid (18 mg, 8%).

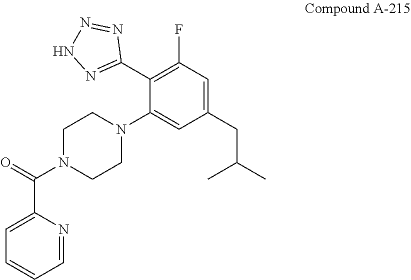 [SMILES: CC(C)Cc1cc(F)c(-c2nn[nH]n2)c(N2CCN(C(=O)c3ccccn3)CC2)c1]

To a stirred solution of intermediate 2 (150 mg, 0.574 mmol) in DMF (10 mL) at 0° C. was added pyridine-2-carboxylic acid (78 mg, 0.631 mmol), followed by EDC·HCl (132 mg, 0.689 mmol), HOBt (93 mg, 0.689 mmol) and DIPEA (0.25 mL, 1.435 mmol). Upon completion of the addition, the reaction mixture was allowed to stir at r.t. for 12 h until complete conversion was observed by TLC. Water was added to the reaction mixture and extraction with EtOAc was performed. The combined organic layers were washed with sat. brine, dried over anhydrous sodium sulfate and evaporated under reduced pressure. The crude residue was purified by silica chromatography (2-3% of MeOH in CH₂Cl₂) to afford 2-fluoro-4-isobutyl-6-[4-(pyridine-2-carbonyl)piperazin-1-yl]benzonitrile as a gummy liquid (170 mg, 81%).

Final tetrazole reaction was performed, by mixing the nitrile (160 mg, 0.437 mmol), NaN₃ (227 mg, 3.49 mmol) and Bu₃SnCl (0.95 mL, 3.49 mmol) in toluene (10 mL) at 140° C. for 14 h in a sealed tube. Aq. work-up, as described in method A, followed by column chromatography (SiO₂, 4-5% of MeOH in CH₂Cl₂) and final trituration of the purified compound with diethyl ether, yielded the targeted tetrazole A-215 as a colorless solid (18 mg, 10%).

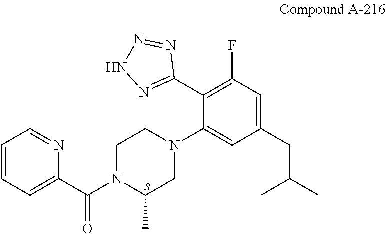

To a stirred solution of N-Boc protected (S)-methyl piperazine (300 mg, 1.50 mmol) in DMF (10 mL) at 0° C. was added pyridine-2-carboxylic acid (203 mg, 1.65 mmol), followed by EDC·HCl (345 mg, 1.80 mmol), HOBt (243 mg, 1.80 mmol) and DIPEA (0.65 mL, 3.74 mmol). Upon completion of the addition, the reaction mixture was allowed to stir at r.t. for 12 h until complete conversion was observed by TLC. Water was added to the reaction mixture and extraction with EtOAc was performed. The combined organic layers were washed with sat. brine, dried over anhydrous sodium sulfate and evaporated under reduced pressure. The crude residue was purified by silica chromatography (2-3% of MeOH in CH₂Cl₂) to afford tert-butyl (3S)-3-methyl-4-(pyridine-2-carbonyl)piperazine-1-carboxylate as a gummy liquid (334 mg, 73%).

Boc-deprotection on 320 mg of the obtained amide (1.05 mmol), using 5 mL of HCl (g) in 1,4-dioxane, afforded after 5 h stirring at room temperature and subsequent trituration in hexane the hydrochloride salt which was used as such in the following reaction step (220 mg crude).

To a stirred solution of [(2S)-2-methylpiperazin-1-yl]-(2-pyridyl)methanone hydrochloride (220 mg, 0.910 mmol) in DMF (10 mL) were added DIPEA (0.40 mL, 2.28 mmol) and K₂CO₃ (252 mg, 1.82 mmol) at 0° C. and the reaction was stirred at r.t. for 10 minutes. Then 2,6-difluoro-4-isobutylbenzonitrile (195 mg, 1.00 mmol) was added at r.t. and the reaction was continued at 65° C. for 12 h. After completion of the reaction was confirmed by TLC, the reaction mixture was diluted with cold water and extraction with EtOAc was performed. The combined organic layers were washed with sat. brine, dried over anhydrous sodium sulfate and evaporated under reduced pressure. The crude thus obtained was purified by column chromatography over silica gel (2-3% MeOH in CH₂Cl₂) to deliver the envisaged nitrile as a gummy liquid (271 mg, 68% over 2 steps).

Final tetrazole reaction was performed, by mixing the nitrile (180 mg, 0.473 mmol), NaN₃ (246 mg, 3.78 mmol) and Bu₃SnCl (1.03 mL, 3.78 mmol) in toluene (10 mL) at 140° C. for 14 h in a sealed tube. Aq. work-up, as described in method A, followed by column chromatography (SiO₂, 4-5% of MeOH in CH₂Cl₂) and ultimate trituration of the purified compound with diethyl ether, yielded the targeted tetrazole A-216 as a colorless solid (18 mg, 9%).

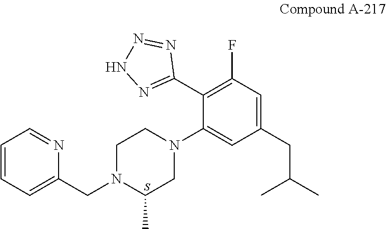

Prepared through method AC by mixing 2-(chloromethyl)pyridine (191 mg, 1.50 mmol), N-Boc protected (S)-methyl piperazine (250 mg, 1.25 mmol) and DIPEA (0.54 mL, 3.12 mmol) in DMF (5 mL) for 12 h at 60° C. An aq. work-up and additional purification using silica chromatography (2-3% MeOH in CH₂Cl₂), yielded tert-butyl (3S)-3-methyl-4-(2-pyridylmethyl)piperazine-1-carboxylate as a gummy liquid (218 mg, 60%).

Boc-deprotection on 210 mg of the alkylated material (0.721 mmol), using 2 mL of HCl (g) in 1,4-dioxane, afforded after 5 h stirring at room temperature and subsequent trituration in hexane the hydrochloride salt which was used as such in the following reaction step (140 mg crude).

To a stirred solution of (2S)-2-methyl-1-(2-pyridylmethyl)piperazine hydrochloride (140 mg, 0.615 mmol) in DMF (10 mL) was added DIPEA (0.27 mL, 1.54 mmol) and K₂CO₃ (170 mg, 1.23 mmol) at 0° C. and the reaction was stirred at r.t. for 10 minutes. Then 2,6-difluoro-4-isobutylbenzonitrile (132 mg, 0.676 mmol) was added at r.t. and the reaction was continued at 65° C. for 12 h. After completion of the reaction was confirmed by TLC, the reaction mixture was diluted with cold water and extraction with EtOAc was performed. The combined organic layers were washed with sat. brine, dried over anhydrous sodium sulfate and evaporated under reduced pressure. The crude thus obtained was purified by column chromatography over silica gel, eluting with 2-3% MeOH in CH₂Cl₂, to afford the desired nitrile as a gummy liquid (120 mg, 45% over 2 steps).

Final tetrazole reaction was performed, by mixing the nitrile (120 mg, 0.327 mmol), NaN₃ (170 mg, 2.62 mmol) and Bu₃SnCl (0.71 mL, 2.62 mmol) in toluene (10 mL) at 145° C. for 14 h in a sealed tube. Aq. work-up, followed by column chromatography (SiO₂, 4-5% of MeOH in CH₂Cl₂) and final trituration of the purified compound with diethyl ether as described in method AC, yielded (2S)-4-[3-fluoro-5-isobutyl-2-(2H-tetrazol-5-yl)phenyl]-2-methyl-1-(2-pyridylmethyl)piperazine as a colorless solid (9 mg, 7%).

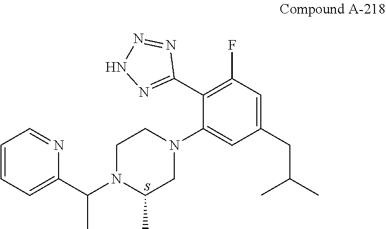

Prepared through method AC by mixing 2-(1-chloroethyl)pyridine (212 mg, 1.50 mmol), N-Boc protected (S)-methyl piperazine (250 mg, 1.25 mmol) and DIPEA (0.54 mL, 3.12 mmol) in DMF (5 mL) for 12 h at 60° C. An aq. work-up and additional purification using silica chromatography (2-3% MeOH in CH₂Cl₂), yielded tert-butyl (3S)-3-methyl-4-[1-(2-pyridyl)ethyl]piperazine-1-carboxylate as a gummy liquid (152 mg, 40%).

Subsequent Boc-deprotection of the alkylated material (150 mg, 0.491 mmol), using 2 mL of HCl (g) in 1,4-dioxane, afforded after 5 h stirring at room temperature and ultimate trituration in hexane the hydrochloride salt which was taken as such to the next reaction step (120 mg crude).

To a stirred solution of (2S)-2-methyl-1-[1-(2-pyridyl)ethyl]piperazine hydrochloride (120 mg, 0.496 mmol) in DMF (10 mL) was added DIPEA (0.22 mL, 1.24 mmol) and K₂CO₃ (137 mg, 0.993 mmol) at 0° C. and the reaction was stirred at r.t. for 10 minutes. Then 2,6-difluoro-4-isobutylbenzonitrile (107 mg, 0.546 mmol) was added at r.t. and the reaction was continued at 65° C. for 12 h. After completion of the reaction was confirmed by TLC, cold water was added to the reaction mixture and extraction with EtOAc was performed. The combined organic layers were washed with sat. brine, dried over anhydrous sodium sulfate and evaporated under reduced pressure. The crude thus obtained was purified by column chromatography over silica gel, eluting with 2-3% MeOH in CH₂Cl₂, to afford the desired nitrile as a gummy liquid (84 mg, 45% over 2 steps).

Final tetrazole reaction was performed, by mixing the nitrile (75 mg, 0.197 mmol), NaN₃ (102 mg, 1.58 mmol) and Bu₃SnCl (0.43 mL, 1.58 mmol) in toluene (10 mL) at 145° C. for 14 h in a sealed tube. Aq. work-up, followed by column chromatography (SiO₂, 4-5% of MeOH in CH₂Cl₂) and final trituration of the purified compound with diethyl ether as described in method AC, yielded (2S)-4-[3-fluoro-5-isobutyl-2-(2H-tetrazol-5-yl)phenyl]-2-methyl-1-[1-(2-pyridyl)ethyl]piperazine as a colorless solid (10 mg, 12%).

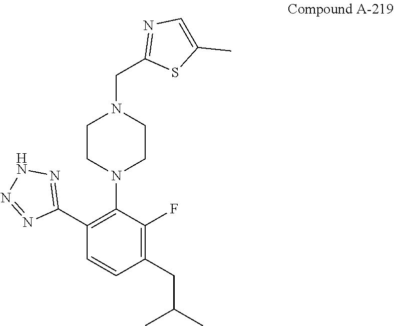 [SMILES: Cc1cnc(CN2CCN(c3c(-c4nn[nH]n4)ccc(CC(C)C)c3F)CC2)s1]

Prepared through method AB by mixing intermediate 39 (120 mg, 0.403 mmol), DIPEA (0.18 mL, 1.01 mmol) and 2-(chloromethyl)-5-methyl-thiazole (72 mg, 0.484 mmol) in DMF (10 mL) for 12 h at 60° C. After performing an aq. work-up, the organic residue was purified by silica chromatography (2-3% MeOH in CH₂Cl₂) to give 3-fluoro-4-isobutyl-2-(4-((5-methylthiazol-2-yl)methyl)piperazin-1-yl)benzonitrile as a gummy solid (63 mg, 42%).

Final tetrazole reaction was performed, by mixing the nitrile (60 mg, 0.161 mmol), NaN₃ (84 mg, 1.29 mmol) and Bu₃SnCl (0.35 mL, 1.29 mmol) in toluene (10 mL) at 145° C. for 14 h in a sealed tube. Aq. work-up, followed by column chromatography and ultimate trituration of the purified compound with diethyl ether afforded the desired tetrazole as a colorless solid (10 mg, 14%).

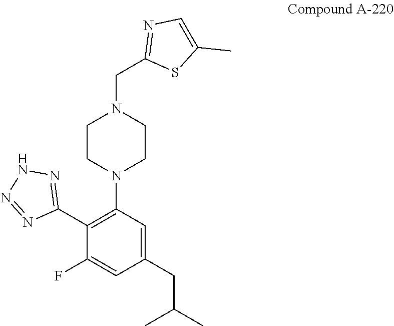 [SMILES: Cc1cnc(CN2CCN(c3cc(CC(C)C)cc(F)c3-c3nn[nH]n3)CC2)s1]

Prepared through method AB by using intermediate 2 (150 mg, 0.574 mmol), DIPEA (0.25 mL, 1.435 mmol) and 2-(chloromethyl)-5-methyl-thiazole (102 mg, 0.689 mmol) in a nucleophilic substitution reaction. The desired compound was obtained after 8 h stirring at 60° C. After completion of the reaction was confirmed by TLC, an aq. work-up as described in method AB was performed, followed by purification via silica chromatography (2-3% MeOH in CH₂Cl₂) to give 2-fluoro-4-isobutyl-6-(4-((5-methylthiazol-2-yl)methyl)piperazin-1-yl)benzonitrile as a gummy liquid (150 mg, 69%).

Final tetrazole reaction was performed, by mixing the nitrile (140 mg, 0.376 mmol), NaN₃ (196 mg, 3.01 mmol) and Bu₃SnCl (0.82 mL, 3.01 mmol) in toluene (10 mL) at 140° C. for 14 h in a sealed tube. Aq. work-up, followed by column chromatography and ultimate trituration of the purified compound with diethyl ether, yielded the envisaged tetrazole as a colorless solid (28 mg, 18%).

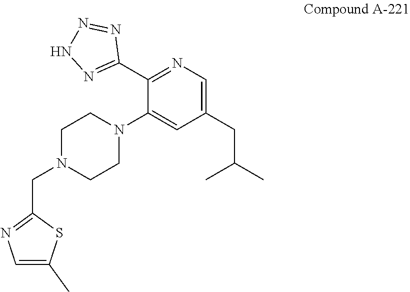 [SMILES: Cc1cnc(CN2CCN(c3cc(CC(C)C)cnc3-c3nn[nH]n3)CC2)s1]

Prepared through method AB by mixing intermediate 47 (75 mg, 0.267 mmol), DIPEA (0.12 mL, 0.668 mmol) and 2-(chloromethyl)-5-methyl-thiazole (47 mg, 0.320 mmol) in a nucleophilic substitution reaction to obtain the desired compound after 12 h stirring at 60° C. After performing an aq. work-up, the organic residue was purified by silica chromatography (2-3% MeOH in CH₂Cl₂) to give 5-isobutyl-3-[4-[(5-methylthiazol-2-yl)methyl]piperazin-1-yl]pyridine-2-carbonitrile as a gummy liquid (50 mg, 36%).

Final tetrazole reaction was performed, by mixing the nitrile (40 mg, 0.113 mmol), NaN₃ (59 mg, 0.901 mmol) and Bu₃SnCl (0.25 mL, 0.901 mmol) in toluene (10 mL) at 145° C. for 14 h in a sealed tube. Aq. work-up, followed by column chromatography and ultimate trituration of the purified compound with diethyl ether yielded the desired 2-((4-(5-isobutyl-2-(2H-tetrazol-5-yl)pyridin-3-yl)piperazin-1-yl)methyl)-5-methylthiazole as a colorless solid (6 mg, 13%).

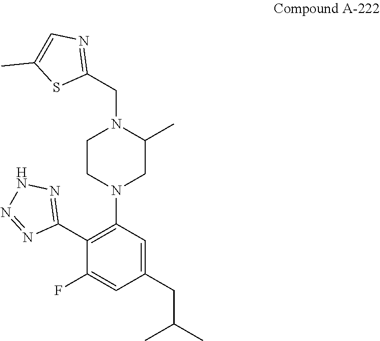 [SMILES: Cc1cnc(CN2CCN(c3cc(CC(C)C)cc(F)c3-c3nn[nH]n3)CC2C)s1]

Prepared through method AC from intermediate 10. To a stirred solution of tert-butyl 3-methylpiperazine-1-carboxylate (200 mg, 1.00 mmol) in DMF (5 mL) was added DIPEA (0.44 mL, 2.50 mmol) at 0° C., after which the reaction was stirred at r.t. for 10 minutes. Then 2-(chloromethyl)-5-methyl-thiazole (177 mg, 1.20 mmol) was added and the reaction was continued at 60° C. for an additional 8 hours. After the completion of the reaction was confirmed by TLC, the reaction mixture was worked up as described in method AC. Subsequent column chromatography over silica gel (eluting with 2-3% MeOH in CH₂Cl₂) afforded tert-butyl 3-methyl-4-[(5-methylthiazol-2-yl)methyl]piperazine-1-carboxylate (252 mg, 81%).

To a stirred solution of tert-butyl 3-methyl-4-[(5-methylthiazol-2-yl)methyl]piperazine-1-carboxylate (230 mg, 0.739 mmol) in 1,4-dioxane (2 mL), HCl (g) in dioxane (3 mL) was added at 0° C. and the reaction was continued at r.t. for 5 h. After the completion of the reaction was confirmed by TLC, the reaction mixture was evaporated to dryness under reduced pressure. Next, the residue was washed with hexane to afford a crude 5-methyl-2-[(2-methylpiperazin-1-yl)methyl]thiazole hydrochloride, which was used as such in the next step without purification (180 mg crude).

To a stirred solution of 5-methyl-2-[(2-methylpiperazin-1-yl)methyl]thiazole hydrochloride (175 mg, 0.706 mmol) in DMF (10 mL) was added DIPEA (0.31 mL, 1.77 mmol) and K₂CO₃ (196 mg, 1.41 mmol) at 0° C. and the reaction was stirred at r.t. for 10 minutes. Then 2,6-difluoro-4-isobutylbenzonitrile (152 mg, 0.777 mmol) was added at r.t. and the reaction was continued at 65° C. for 14 h. After the completion of the reaction was confirmed by TLC, cold water was added to the reaction mixture and extraction with EtOAc was performed. The combined organic layers were washed with sat. brine, dried over anhydrous sodium sulfate and evaporated under reduced pressure. The obtained crude residue was purified by column chromatography over silica gel (2-3% MeOH in CH₂Cl₂) to afford the envisaged product 2-fluoro-4-isobutyl-6-[3-methyl-4-[(5-methylthiazol-2-yl)methyl]piperazin-1-yl]benzonitrile as a gummy liquid (218 mg, 76% over 2 steps).

A mixture of this isolated nitrile (200 mg, 0.517 mmol), NaN₃ (269 mg, 4.14 mmol) and Bu₃SnCl (1.12 mL, 4.14 mmol) in toluene (10 mL) was stirred at 140° C. for 14 h in a sealed tube. After the completion of the reaction was confirmed by TLC, the reaction mixture was concentrated in vacuo. The residue was re-dissolved in CH₂Cl₂ and washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The obtained crude was purified by silica chromatography (4-5% of MeOH in CH₂Cl₂), followed by trituration with diethyl ether, to afford 2-[[4-[3-fluoro-5-isobutyl-2-(2H-tetrazol-5-yl)phenyl]-2-methyl-piperazin-1-yl]methyl]-5-methyl-thiazole as a colorless solid (20 mg, 9%).

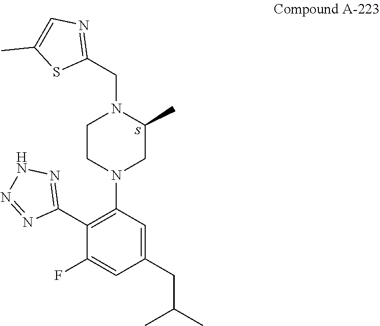

Prepared through method AC from intermediate 10. To a stirred solution of N-Boc protected (S)-methyl piperazine (200 mg, 1.00 mmol) in DMF (5 mL) was added DIPEA (0.44 mL, 2.50 mmol) at 0° C., after which the reaction was stirred at r.t. for 10 minutes. Then 2-(chloromethyl)-5-methyl-thiazole (177 mg, 1.20 mmol) was added and the reaction was continued at 60° C. for 8 h. After the completion of the reaction was confirmed by TLC, cold water was added and the reaction mixture was extracted with EtOAc. The combined organic layers were washed with sat. brine, dried over anhydrous sodium sulfate and evaporated under reduced pressure. The obtained crude residue was purified by column chromatography over silica gel (eluting with 2-3% MeOH in CH₂Cl₂) to afford (S)-tert-butyl 3-methyl-4-[(5-methylthiazol-2-yl)methyl]piperazine-1-carboxylate (258 mg, 83%).

To a stirred solution of (S)-tert-butyl 3-methyl-4-[(5-methylthiazol-2-yl)methyl]piperazine-1-carboxylate (230 mg, 0.739 mmol) in 1,4-dioxane (2 mL), HCl (g) in dioxane (3 mL) was added at 0° C. and the reaction was continued at r.t. for 5 h. After the completion of the reaction was confirmed by TLC, the reaction mixture was evaporated to dryness under reduced pressure, then washed with hexane to afford a crude residue 5-methyl-2-[[(2S)-2-methylpiperazin-1-yl]methyl]thiazole hydrochloride. The crude compound thus obtained was taken to the next step without purification (184 mg crude).

To a stirred solution of 5-methyl-2-[[(2S)-2-methylpiperazin-1-yl]methyl]thiazole hydrochloride (180 mg, 0.726 mmol) in DMF (5 mL) was added DIPEA (0.32 mL, 1.82 mmol) and K₂CO₃ (201 mg, 1.45 mmol) at 0° C. and the reaction was stirred at r.t. for 10 minutes. Then 2,6-difluoro-4-isobutylbenzonitrile (156 mg, 0.799 mmol) was added at r.t. and the reaction was continued at 65° C. for 14 h. After the completion of the reaction was confirmed by TLC, the reaction mixture was diluted with cold water and extraction with EtOAc was performed. The combined organic layers were washed with sat. brine, dried over anhydrous sodium sulfate and evaporated under reduced pressure. The crude thus obtained was purified by column chromatography over silica gel (eluting with 2-3% MeOH in CH₂Cl₂) to afford the envisaged product 2-fluoro-4-isobutyl-6-[(3S)-3-methyl-4-[(5-methylthiazol-2-yl)methyl]piperazin-1-yl]benzonitrile as a gummy liquid (215 mg, 75% over 2 steps).

A mixture of this isolated nitrile (190 mg, 0.492 mmol), NaN₃ (256 mg, 3.93 mmol) and Bu₃SnCl (1.07 mL, 3.93 mmol) in toluene (10 mL) was stirred at 140° C. for 14 h in a sealed tube. After the completion of the reaction was confirmed by TLC, the reaction mixture was evaporated under reduced pressure. The residue was re-dissolved in CH₂Cl₂ and washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The crude compound thus obtained was purified by silica chromatography (4-5% of MeOH in CH₂Cl₂) to afford 2-[[(2S)-4-[3-fluoro-5-isobutyl-2-(2H-tetrazol-5-yl)phenyl]-2-methyl-piperazin-1-yl]methyl]-5-methyl-thiazole which was further triturated with diethyl ether to obtain a colorless solid (23 mg, 11%).

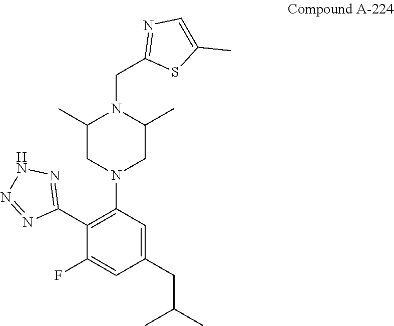 [SMILES: Cc1cnc(CN2C(C)CN(c3cc(CC(C)C)cc(F)c3-c3nn[nH]n3)CC2C)s1]

Prepared through method AD. To a stirred solution of tert-butyl 2,6-dimethylpiperazine-1-carboxylate (463 mg, 2.16 mmol) in DMF (10 mL) was added DIPEA (0.78 mL, 4.50 mmol) at 0° C., after which the reaction was stirred at r.t. for 10 minutes. Then 4-bromo-2,6-difluorobenzonitrile (392 mg, 1.80 mmol) was added and the reaction was continued at 60° C. for 8 h. After the completion of the reaction was confirmed by TLC, cold water was added to the reaction mixture and extraction with EtOAc was performed. The combined organic layers were washed with sat. brine, dried over anhydrous sodium sulfate and evaporated under reduced pressure. The obtained crude residue was purified by column chromatography over silica gel (eluting with 2-3% MeOH in CH₂Cl₂) to afford tert-butyl 4-(5-bromo-2-cyano-3-fluorophenyl)-2,6-dimethylpiperazine-1-carboxylate (519 mg, 70%).

To a stirred solution of tert-butyl 4-(5-bromo-2-cyano-3-fluorophenyl)-2,6-dimethylpiperazine-1-carboxylate (510 mg, 1.24 mmol) in 1,4-dioxane (10 mL) was added 4,4,5,5-tetramethyl-2-(2-methylprop-1-en-1-yl)-1,3,2-dioxaborolane (270 mg, 1.48 mmol), followed by K₂CO₃ (427 mg, 3.09 mmol) and the resultant mixture was bubbled with argon for 20 min. Then Pd(dppf)Cl₂ (91 mg, 0.124 mmol) was added after which the reaction was heated to 80° C. for 6 h until completion of the reaction was observed by TLC. The reaction mixture was concentrated under reduced pressure to remove the volatiles and the residue was re-dissolved with ethyl acetate and washed with water and sat. brine. Subsequent drying over anhydrous sodium sulfate and evaporation under reduced pressure afforded the compound of interest as a colorless gum (407 mg, 85%).

A stirred solution of tert-butyl 4-(2-cyano-3-fluoro-5-(2-methylprop-1-en-1-yl)phenyl)-2,6-dimethylpiperazine-1-carboxylate (405 mg, 1.05 mmol) in MeOH (5 mL) was hydrogenated over 10% Pd/C (0.040 g) under 5 Kg/cm² H₂ pressure using a Parr hydrogenator for 3 h at ambient temperature. After confirming the completion of the reaction by LC-MS, the reaction mixture was filtered through a Celite® bed and was evaporated under reduced pressure to afford the crude hydrogenated compound. Further purification using silica chromatography (40-45% EtOAc in hexane) yielded the envisaged compound as a colorless gum (392 mg, 96%).

To a stirred solution of tert-butyl 4-(2-cyano-3-fluoro-5-isobutylphenyl)-2,6-dimethylpiperazine-1-carboxylate (390 mg, 1.00 mmol) in 1,4-dioxane (2 mL) was added HCl (g) in dioxane (3 mL) at 0° C. Upon completion of the addition, the reaction was allowed to slowly warm up to room temperature and kept stirring at r.t. for 5 h until complete conversion was obtained (via TLC). The reaction mixture was concentrated in vacuo, followed by washing of the crude residue with hexane. The crude compound was taken to the next step without additional purification (330 mg crude).

To a stirred solution of the deprotected compound (150 mg, 0.460 mmol) in DMF (3 mL) at 0° C. was added DIPEA (0.24 mL, 1.38 mmol) dropwise, after which the reaction was continued at room temperature for 10 minutes. Subsequently, 2-(chloromethyl)-5-methyl-thiazole (82 mg, 0.552 mmol) was added and the reaction was kept stirring at 80° C. for 14 h. After completion of the reaction was confirmed by TLC, cold water was added to the reaction mixture and extraction with CH₂Cl₂ was performed. The combined organic layers were washed with sat. brine, dried over anhydrous sodium sulfate and evaporated under reduced pressure. The crude obtained was purified by column chromatography over silica gel (4-5% MeOH in CH₂Cl₂), affording 2-[3,5-dimethyl-4-[(5-methylthiazol-2-yl)methyl]piperazin-1-yl]-6-fluoro-4-isobutyl-benzonitrile as an off-white solid (110 mg, 59%).

A mixture of the intermediate nitrile (100 mg, 0.25 mmol), NaN₃ (130 mg, 2.00 mmol) and Bu₃SnCl (0.54 mL, 2.00 mmol) in toluene (8 mL) was stirred at 145° C. for 14 h in a sealed tube. After the completion of the reaction was confirmed by TLC, an aq. work-up as described in method AD was performed. The crude compound was purified by silica chromatography (4-5% of MeOH in CH₂Cl₂), followed by trituration with diethyl ether to afford the targeted tetrazole as a colorless solid (5 mg, 4%).

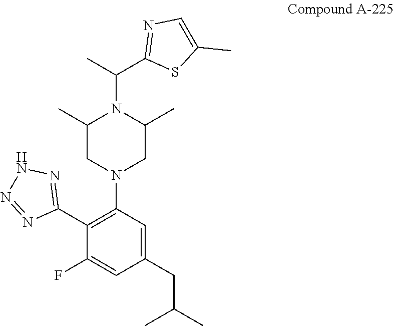 [SMILES: Cc1cnc(C(C)N2C(C)CN(c3cc(CC(C)C)cc(F)c3-c3nn[nH]n3)CC2C)s1]

Prepared through method AB by adding DIPEA (0.25 mL, 1.435 mmol) and 2-(1-chloroethyl)-5-methyl-thiazole (111 mg, 0.689 mmol) to a solution of intermediate 2 (150 mg, 0.574 mmol) in DMF (10 mL). The resulting reaction mixture was stirred at 60° C. for 8 h. After performing an aq. work-up with EtOAc, the organic residue was purified by silica chromatography (2-3% MeOH in CH₂Cl₂) to give 2-fluoro-4-isobutyl-6-[4-[1-(5-methylthiazol-2-yl)ethyl]piperazin-1-yl]benzonitrile as a gummy liquid (143 mg, 64%).

Final tetrazole reaction was performed, by mixing the nitrile (105 mg, 0.272 mmol), NaN₃ (142 mg, 2.17 mmol) and Bu₃SnCl (0.59 mL, 2.17 mmol) in toluene (10 mL) at 140° C. for 14 h in a sealed tube. Aq. work-up followed by column chromatography and final trituration with diethyl ether yielded the desired tetrazole as a colorless solid (22 mg, 19%).

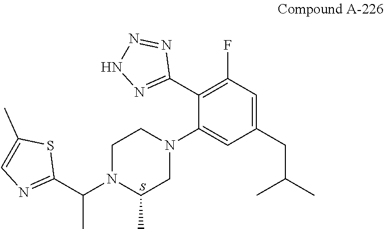

Prepared through method AC from intermediate 10. To a stirred solution of N-Boc protected (S)-methyl piperazine (300 mg, 1.50 mmol) in DMF (5 mL) was added DIPEA (0.65 mL, 3.75 mmol) at 0° C., after which the reaction was stirred at r.t. for 10 minutes. Then 2-(1-chloroethyl)-5-methyl-thiazole (291 mg, 1.80 mmol) was added and the reaction was continued at 60° C. for 8 h. After the completion of the reaction was confirmed by TLC, cold water was added and the reaction mixture was extracted with EtOAc. The combined organic layers were washed with sat. brine, dried over anhydrous sodium sulfate and evaporated under reduced pressure. The obtained crude residue was purified by column chromatography over silica gel (eluting with 2-3% MeOH in CH₂Cl₂) to afford (3S)-tert-butyl 3-methyl-4-[(5-methylthiazol-2-yl)ethyl]piperazine-1-carboxylate (168 mg, 34%).

To a stirred solution of (3S)-tert-butyl 3-methyl-4-[(5-methylthiazol-2-yl)ethyl]piperazine-1-carboxylate (165 mg, 0.507 mmol) in 1,4-dioxane (1 mL), HCl (g) in dioxane (2 mL) was added at 0° C. and the reaction was continued at r.t. for 5 h. After the completion of the reaction was confirmed by TLC, the reaction mixture was evaporated to dryness under reduced pressure, then washed with hexane to afford a crude residue. The crude hydrochloride thus obtained was taken to the next step without additional purification (140 mg crude).

To a stirred solution of 5-methyl-2-[1-[(2S)-2-methylpiperazin-1-yl]ethyl]thiazole hydrochloride (140 mg, 0.535 mmol) in DMF (5 mL) was added DIPEA (0.23 mL, 1.34 mmol) and K₂CO₃ (148 mg, 1.07 mmol) at 0° C. and the reaction was stirred at r.t. for 10 minutes. Then 2,6-difluoro-4-isobutylbenzonitrile (115 mg, 0.588 mmol) was added at r.t. and the reaction was continued at 65° C. for 14 h. After the completion of the reaction was confirmed by TLC, the reaction mixture was diluted with cold water and extraction with EtOAc was performed. The combined organic layers were washed with sat. brine, dried over anhydrous sodium sulfate and evaporated under reduced pressure. The crude thus obtained was purified by column chromatography over silica gel eluting with 2-3% MeOH in CH₂Cl₂ to afford the envisaged product 2-fluoro-4-isobutyl-6-[(3S)-3-methyl-4-[1-(5-methylthiazol-2-yl)ethyl]piperazin-1-yl]benzonitrile as a gummy liquid (81 mg, 40% over 2 steps).

A mixture of this isolated nitrile (70 mg, 0.175 mmol), NaN₃ (91 mg, 1.40 mmol) and Bu₃SnCl (0.38 mL, 1.40 mmol) in toluene (10 mL) was stirred at 145° C. for 14 h in a sealed tube. After the completion of the reaction was confirmed by TLC, the reaction mixture was evaporated under reduced pressure. The residue was re-dissolved in CH₂Cl₂ and washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The crude compound thus obtained was purified by silica chromatography (4-5% of MeOH in CH₂Cl₂) to afford 2-[1-[(2S)-4-[3-fluoro-5-isobutyl-2-(2H-tetrazol-5-yl)phenyl]-2-methyl-piperazin-1-yl]ethyl]-5-methyl-thiazole which was triturated with diethyl ether to obtain a colorless solid (7 mg, 9%).

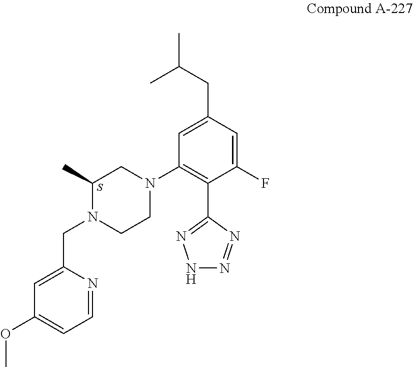

Prepared through method AC by mixing 2-(chloromethyl)-4-methoxy-pyridine (5.95 g, 37.7 mmol), N-Boc protected (S)-methyl piperazine (6.30 g, 31.5 mmol) and DIPEA (13.7 mL, 78.6 mmol) in DMF (65 mL) for 14 h at 60° C. An aq. work-up and additional purification using silica chromatography (2-3% MeOH in CH₂Cl₂), as described in method AC, delivered tert-butyl (3S)-4-[(4-methoxy-2-pyridyl)methyl]-3-methyl-piperazine-1-carboxylate as a gummy liquid (6.98 g, 69%).

Subsequent Boc-deprotection (on 6.4 g scale) using 80 mL of HCl (g) in 1,4-dioxane afforded, after 5 h stirring at room temperature and trituration in hexane, the desired hydrochloride salt which was used as such in the following SNAr reaction (5.6 g crude).

To a stirred solution of (2S)-1-[(4-methoxy-2-pyridyl)methyl]-2-methyl-piperazine hydrochloride (5.47 g, 19.8 mmol) in DMF (50 mL) was added DIPEA (8.6 mL, 49.5 mmol) and K₂CO₃ (5.58 g, 39.6 mmol) at 0° C. and the reaction was stirred at r.t. for 10 minutes. Then 2,6-difluoro-4-isobutylbenzonitrile (4.25 g, 21.8 mmol) was added at r.t. and the reaction was continued at 65° C. for 14 h. After completion of the reaction was confirmed by TLC, the reaction mixture was diluted with cold water and extraction with EtOAc was performed. The combined organic layers were washed with sat. brine, dried over anhydrous sodium sulfate and evaporated under reduced pressure. The crude thus obtained was purified by column chromatography over silica gel eluting with 2-3% MeOH in CH₂Cl₂ to afford the envisaged nitrile as a gummy liquid (4.85 g, 61% over 2 steps).

Final tetrazole reaction was performed, by mixing the nitrile (4.80 g, 12.1 mmol), NaN₃ (6.29 g, 96.8 mmol) and Bu₃SnCl (26.3 mL, 96.8 mmol) in toluene (100 mL) at 150° C. for 14 h in a sealed tube. Aq. work-up, followed by column chromatography (SiO₂, 4-5% of MeOH in CH₂Cl₂) and ultimate trituration of the purified compound with diethyl ether yielded (2S)-4-[3-fluoro-5-isobutyl-2-(2H-tetrazol-5-yl)phenyl]-1-[(4-methoxy-2-pyridyl)methyl]-2-methyl-piperazine as a colorless solid (2.39 g, 45%).

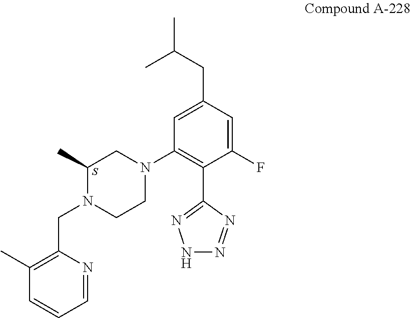

Prepared through method AC by mixing 2-(chloromethyl)-3-methyl-pyridine (5.94 g, 41.9 mmol), N-Boc protected (S)-methyl piperazine (7.00 g, 35.0 mmol) and DIPEA (15.2 mL, 87.4 mmol) in DMF (70 mL) for 14 h at 60° C. An aq. work-up and additional purification using silica chromatography (2-3% MeOH in CH₂Cl₂), yielded tert-butyl (3S)-3-methyl-4-[(3-methyl-2-pyridyl)methyl]piperazine-1-carboxylate as a gummy liquid (7.31 g, 69%).

Boc-deprotection on 7.3 g, using 80 mL of HCl (g) in 1,4-dioxane, afforded after 5 h stirring at room temperature and subsequent trituration in hexane the hydrochloride salt which was used as such in the following reaction step (6.8 g crude).

To a stirred solution of (2S)-2-methyl-1-[(3-methyl-2-pyridyl)methyl]piperazine hydrochloride (6.64 g, 27.5 mmol) in DMF (60 mL) was added DIPEA (12.0 mL, 68.7 mmol) and K₂CO₃ (7.59 g, 54.9 mmol) at 0° C. and the reaction was stirred at r.t. for 10 minutes. Then 2,6-difluoro-4-isobutylbenzonitrile (5.90 g, 30.2 mmol) was added at r.t. and the reaction was continued at 65° C. for 14 h. After the completion of the reaction was confirmed by TLC, cold water was added to the reaction mixture and extraction with EtOAc was performed. The combined organic layers were washed with sat. brine, dried over anhydrous sodium sulfate and evaporated under reduced pressure. The crude thus obtained was purified by column chromatography over silica gel, eluting with 2-3% MeOH in CH₂Cl₂, to afford the desired nitrile as a gummy liquid (3.98 g, 44% over 2 steps).

Final tetrazole reaction was performed, by mixing the nitrile (3.90 g, 10.2 mmol), NaN₃ (5.33 g, 82.0 mmol) and Bu₃SnCl (22.2 mL, 82.0 mmol) in toluene (90 mL) at 150° C. for 14 h in a sealed tube. Aq. work-up, followed by column chromatography (SiO₂, 4-5% of MeOH in CH₂Cl₂) and final trituration of the purified compound with diethyl ether as described in method AC, yielded (2S)-4-[3-fluoro-5-isobutyl-2-(2H-tetrazol-5-yl)phenyl]-2-methyl-1-[(3-methyl-2-pyridyl)methyl]piperazine as a colorless solid (1.35 g, 31%).

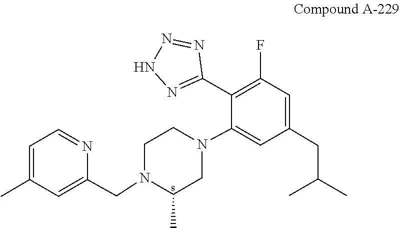 [SMILES: Cc1ccnc(CN2CCN(c3cc(CC(C)C)cc(F)c3-c3nn[nH]n3)C[C@@H]2C)c1]

Prepared through method AC by mixing 2-(chloromethyl)-4-methyl-pyridine (4.16 g, 29.4 mmol), N-Boc protected (S)-methyl piperazine (4.90 g, 24.5 mmol) and DIPEA (10.7 mL, 61.2 mmol) in DMF (50 mL) for 14 h at 60° C. An aq. work-up and additional purification using silica chromatography (2-3% MeOH in CH₂Cl₂), yielded tert-butyl (3S)-3-methyl-4-[(4-methyl-2-pyridyl)methyl]piperazine-1-carboxylate as a gummy liquid (4.63 g, 62%).

Next, Boc-deprotection on 4.6 g scale was performed using 80 mL of HCl (g) in 1,4-dioxane to afford after 5 h stirring at room temperature and subsequent trituration in hexane the hydrochloride salt which was taken as such to the next step (3.5 g crude).

To a stirred solution of (2S)-2-methyl-1-[(4-methyl-2-pyridyl)methyl]piperazine hydrochloride (3.50 g, 14.5 mmol) in DMF (40 mL) was added DIPEA (6.3 mL, 36.2 mmol) and K₂CO₃ (4.00 g, 29.0 mmol) at 0° C. and the reaction was stirred at r.t. for 10 minutes. Then 2,6-difluoro-4-isobutylbenzonitrile (3.11 g, 15.9 mmol) was added at r.t. and the reaction was continued at 65° C. for 14 h. After the completion of the reaction was confirmed by TLC, cold water was added to the reaction mixture after which the obtained solution was extracted with EtOAc. The combined organic layers were washed with sat. brine, dried over anhydrous sodium sulfate and evaporated under reduced pressure. The crude thus obtained was purified by column chromatography over silica gel (eluting with 2-3% MeOH in CH₂Cl₂) to afford the desired nitrile as a gummy liquid (3.38 g, 59% over 2 steps).

Final tetrazole reaction was performed, by mixing the nitrile (3.30 g, 8.67 mmol), NaN₃ (4.51 g, 6.94 mmol) and Bu₃SnCl (18.8 mL, 6.94 mmol) in toluene (100 mL) at 150° C. for 14 h in a sealed tube. Aq. work-up, followed by column chromatography (SiO₂, 4-5% of MeOH in CH₂Cl₂) and ultimate trituration of the purified compound with diethyl ether as described in method AC, yielded (2S)-4-[3-fluoro-5-isobutyl-2-(2H-tetrazol-5-yl)phenyl]-2-methyl-1-[(4-methyl-2-pyridyl)methyl]piperazine as a colorless solid (1.87 g, 51%).

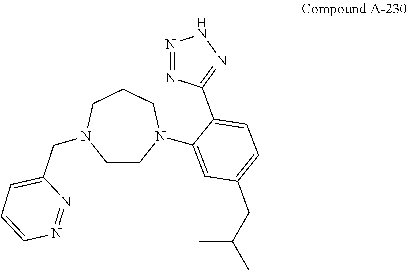 [SMILES: CC(C)Cc1ccc(-c2nn[nH]n2)c(N2CCCN(Cc3cccnn3)CC2)c1]

Prepared in a similar manner as described in method D from intermediate 53 (120 mg, 0.408 mmol) and 3-(chloromethyl)pyridazine (79 mg, 0.613 mmol). The alkylation reaction was completed after 14 h at 70° C. After performing an aq. work-up as described in method D, the organic residue was purified by silica chromatography (4-5% MeOH in CH₂Cl₂) to afford 4-isobutyl-2-[4-(pyridazin-3-ylmethyl)-1,4-diazepan-1-yl]benzonitrile as an off-white solid (100 mg, 70%).

Final tetrazole reaction was performed, by mixing the nitrile (100 mg, 0.286 mmol), sodium azide (149 mg, 2.29 mmol) and Bu₃SnCl (0.62 mL, 2.29 mmol) in toluene (15 mL) at 150° C. for 14 h in a sealed tube. Aq. work-up, followed by column chromatography (4-5% MeOH in CH₂Cl₂) and subsequent trituration of the purified compound with diethyl ether, yielded the envisaged tetrazole A-230 as an off-white solid (20 mg, 17%).

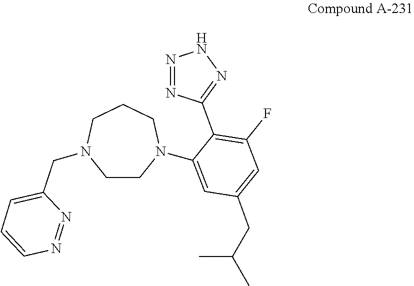 [SMILES: CC(C)Cc1cc(F)c(-c2nn[nH]n2)c(N2CCCN(Cc3cccnn3)CC2)c1]

Prepared in a similar fashion as described in method G from intermediate 10. To a stirred solution of tert-butyl 1,4-diazepane-1-carboxylate (250 mg, 1.25 mmol) in DMF (5 mL) was added DIPEA (0.65 mL, 3.75 mmol) at 0° C., after which the reaction was stirred at r.t. for 10 minutes. Then 3-(chloromethyl)pyridazine (193 mg, 1.50 mmol) was added and the reaction was continued at 60° C. for an additional 12 hours. After the completion of the reaction was confirmed by TLC, the reaction mixture was worked up as described in method G. Subsequent column chromatography over silica gel (eluting with 2-3% MeOH in CH₂Cl₂) afforded tert-butyl 4-(pyridazin-3-ylmethyl)-1,4-diazepane-1-carboxylate as a gummy liquid (330 mg, 90%).

To a stirred solution of tert-butyl 4-(pyridazin-3-ylmethyl)-1,4-diazepane-1-carboxylate (200 mg, 0.68 mmol) in 1,4-dioxane (2 mL), HCl (g) in dioxane (3 mL) was added at 0° C. and the reaction was continued at r.t. for 5 h. After the completion of the reaction was confirmed by TLC, the reaction mixture was evaporated to dryness under reduced pressure. Next, the residue was washed with hexane to afford a crude 1-(pyridazin-3-ylmethyl)-1,4-diazepane hydrochloride, which was used as such in the next step without further purification (150 mg crude).

To a stirred solution of 1-(pyridazin-3-ylmethyl)-1,4-diazepane hydrochloride (150 mg, 0.66 mmol) in DMF (10 mL) was added K₂CO₃ (227 mg, 1.64 mmol) at 0° C. and the reaction was stirred at r.t. for 10 minutes. Then 2,6-difluoro-4-isobutylbenzonitrile (141 mg, 0.72 mmol) was added at r.t. and the reaction was continued at 65° C. for 12 h. After the completion of the reaction was confirmed by TLC, water was added to the reaction mixture and extraction with EtOAc was performed. The combined organic layers were washed with sat. brine, dried over anhydrous sodium sulfate and evaporated under reduced pressure. The obtained crude residue was purified by column chromatography over silica gel (2-3% MeOH in CH₂Cl₂) to afford the envisaged product 2-fluoro-4-isobutyl-6-[4-(pyridazin-3-ylmethyl)-1,4-diazepan-1-yl]benzonitrile (207 mg, 50% over 2 steps).

A mixture of this isolated nitrile (100 mg, 0.272 mmol), NaN₃ (142 mg, 2.18 mmol) and Bu₃SnCl (0.59 mL, 2.18 mmol) in toluene (10 mL) was stirred at 140° C. for 14 h in a sealed tube. After the completion of the reaction was confirmed by TLC, the reaction mixture was concentrated in vacuo. The residue was re-dissolved in CH₂Cl₂ and washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The obtained crude was purified by silica chromatography (4-5% of MeOH in CH₂Cl₂), followed by trituration with diethyl ether, to afford 1-[3-fluoro-5-isobutyl-2-(2H-tetrazol-5-yl)phenyl]-4-(pyridazin-3-ylmethyl)-1,4-diazepane as a colorless solid (13 mg, 12%).

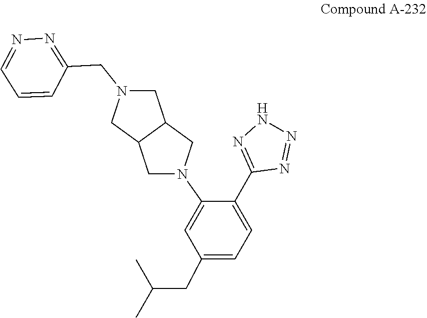 [SMILES: CC(C)Cc1ccc(-c2nn[nH]n2)c(N2CC3CN(Cc4cccnn4)CC3C2)c1]

Prepared in a similar manner as described in method D from intermediate 54 (135 mg, 0.441 mmol) and 3-(chloromethyl)pyridazine (85 mg, 0.662 mmol). The alkylation reaction was completed after 14 h at 70° C. After performing an aq. work-up as described in method D, the crude residue was purified by silica chromatography (2-3% MeOH in CH₂Cl₂) to afford 4-isobutyl-2-[2-(pyridazin-3-ylmethyl)-1,3,3a,4,6,6a-hexahydropyrrolo[3,4-c]pyrrol-5-yl]benzonitrile as a pale-yellow solid (92 mg, 58%).

Final tetrazole reaction was performed, by mixing the nitrile (90 mg, 0.249 mmol), sodium azide (129 mg, 1.99 mmol) and Bu₃SnCl (0.54 mL, 1.99 mmol) in toluene (10 mL) at 150° C. for 14 h in a sealed tube. Aq. work-up, followed by column chromatography (4-5% MeOH in CH₂Cl₂) and ultimate trituration of the purified compound with diethyl ether, afforded the desired tetrazole A-232 as a colorless solid (11 mg, 11%).

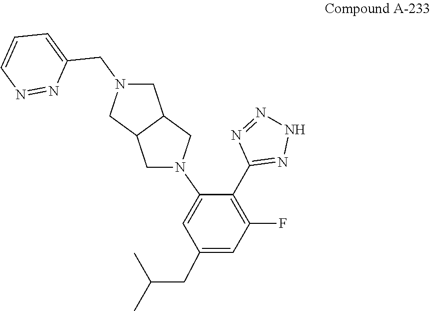 [SMILES: CC(C)Cc1cc(F)c(-c2nn[nH]n2)c(N2CC3CN(Cc4cccnn4)CC3C2)c1]

Prepared following a similar route as described in method G from intermediate 10. To a stirred solution of tert-butyl 2,3,3a,4,6,6a-hexahydro-1H-pyrrolo[3,4-c]pyrrole-5-carboxylate (300 mg, 1.41 mmol) in DMF (5 mL) was added DIPEA (0.74 mL, 4.24 mmol) at 0° C., after which the reaction was stirred at r.t. for 10 minutes. Then 3-(chloromethyl)pyridazine (218 mg, 1.70 mmol) was added and the reaction was continued at 60° C. for an additional 12 hours. After the completion of the reaction was confirmed by TLC, the reaction mixture was worked up as described in method G. Subsequent column chromatography over silica gel (eluting with 2-3% MeOH in CH₂Cl₂) afforded tert-butyl 2,3,3a,4,6,6a-hexahydro-1H-pyrrolo[3,4-c]pyrrole-5-carboxylate as a gummy liquid (375 mg, 87%).

To a stirred solution of tert-butyl 2,3,3a,4,6,6a-hexahydro-1H-pyrrolo[3,4-c]pyrrole-5-carboxylate (350 mg, 1.15 mmol) in 1,4-dioxane (3 mL), HCl (g) in dioxane (5 mL) was added at 0° C. and the reaction was continued at r.t. for 5 h. After the completion of the reaction was confirmed by TLC, the reaction mixture was evaporated to dryness under reduced pressure. Next, the residue was washed with hexane to afford a crude 5-(pyridazin-3-ylmethyl)-2,3,3a,4,6,6a-hexahydro-1H-pyrrolo[3,4-c]pyrrole hydrochloride, which was used as such in the next step without additional purification (260 mg crude).

To a stirred solution of 5-(pyridazin-3-ylmethyl)-2,3,3a,4,6,6a-hexahydro-1H-pyrrolo[3,4-c]pyrrole hydrochloride (260 mg, 1.08 mmol) in DMF (10 mL) was added DIPEA (0.47 mL, 2.70 mmol) and K₂CO₃ (373 mg, 2.70 mmol) at 0° C. and the reaction was stirred at r.t. for 10 minutes. Then 2,6-difluoro-4-isobutylbenzonitrile (232 mg, 1.19 mmol) was added at r.t. and the reaction was continued at 65° C. for 12 h. After the completion of the reaction was confirmed by TLC, extraction with water and EtOAc was performed. The combined organic layers were washed with sat. brine, dried over anhydrous sodium sulfate and evaporated under reduced pressure. The obtained crude residue was purified by column chromatography over silica gel (2-3% MeOH in CH₂Cl₂) to afford 2-fluoro-4-isobutyl-6-[2-(pyridazin-3-ylmethyl)-1,3,3a,4,6,6a-hexahydropyrrolo[3,4-c]pyrrol-5-yl]benzonitrile as a gummy liquid (144 mg, 33% over 2 steps).

A mixture of this isolated nitrile (120 mg, 0.316 mmol), NaN₃ (164 mg, 2.53 mmol) and Bu₃SnCl (0.69 mL, 2.53 mmol) in toluene (10 mL) was stirred at 140° C. for 14 h in a sealed tube. After the completion of the reaction was confirmed by TLC, the reaction mixture was concentrated in vacuo. The residue was re-dissolved in CH₂Cl₂ and washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The obtained crude residue was purified by silica chromatography (4-5% of MeOH in CH₂Cl₂), followed by trituration with diethyl ether, to afford 5-[3-fluoro-5-isobutyl-2-(2H-tetrazol-5-yl)phenyl]-2-(pyridazin-3-ylmethyl)-1,3,3a,4,6,6a-hexahydropyrrolo[3,4-c]pyrrole as a colorless solid (15 mg, 11%).

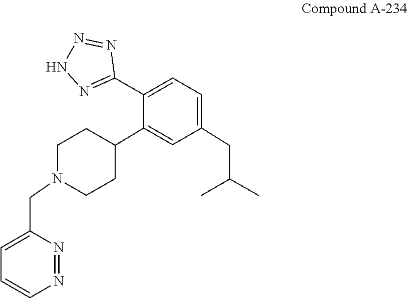 [SMILES: CC(C)Cc1ccc(-c2nn[nH]n2)c(C2CCN(Cc3cccnn3)CC2)c1]

Prepared in a similar fashion as described in method D from intermediate 51 (130 mg, 0.466 mmol) and 3-(chloromethyl)pyridazine (90 mg, 0.699 mmol). The alkylation reaction was completed after 15 h at 70° C. After performing an aq. work-up as described in method D, the organic residue was purified by silica chromatography (2-3% MeOH in CH₂Cl₂) to afford 4-isobutyl-2-[1-(pyridazin-3-ylmethyl)-4-piperidyl]benzonitrile as a gummy solid (110 mg, 71%).

Final tetrazole reaction was performed, by mixing the nitrile (100 mg, 0.299 mmol), sodium azide (155 mg, 2.39 mmol) and Bu₃SnCl (0.65 mL, 2.39 mmol) in toluene (10 mL) at 140° C. for 14 h in a sealed tube. Aq. work-up, followed by column chromatography (4-5% MeOH in CH₂Cl₂) and subsequent trituration of the purified compound with diethyl ether, yielded the envisaged tetrazole A-234 as an off-white solid (13 mg, 11%).

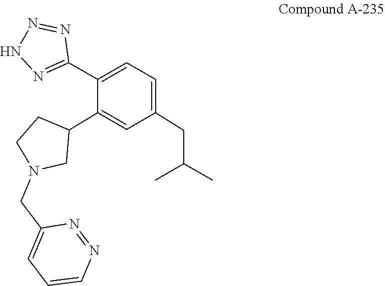 [SMILES: CC(C)Cc1ccc(-c2nn[nH]n2)c(C2CCN(Cc3cccnn3)C2)c1]

Prepared through a similar protocol as for compound A-234, from intermediate 52 (110 mg, 0.415 mmol) and 3-(chloromethyl)pyridazine (80 mg, 0.623 mmol). The alkylation reaction was completed after 15 h at 70° C. After performing an aq. work-up as described in method D, the organic residue was purified by silica chromatography (2-3% MeOH in CH₂Cl₂) to afford 4-isobutyl-2-[1-(pyridazin-3-ylmethyl)pyrrolidin-3-yl]benzonitrile as a gummy solid (9 mg, 71%).

Final tetrazole reaction was performed, by mixing the nitrile (90 mg, 0.281 mmol), sodium azide (146 mg, 2.25 mmol) and Bu₃SnCl (0.61 mL, 2.25 mmol) in toluene (10 mL) at 145° C. for 14 h in a sealed tube. Aq. work-up and subsequent column chromatography (4-5% MeOH in CH₂Cl₂) as described in method D, followed by ultimate trituration of the purified compound with diethyl ether, afforded the desired tetrazole A-235 as a colorless solid (10 mg, 10%).

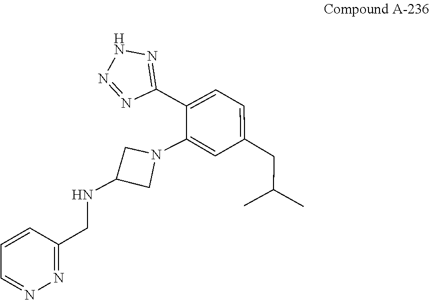 [SMILES: CC(C)Cc1ccc(-c2nn[nH]n2)c(N2CC(NCc3cccnn3)C2)c1]

Prepared in a similar manner as described in method W. To a stirred solution of intermediate 55 (250 mg, 1.09 mmol) in EtOH at 0° C. was added pyridazine-3-carbaldehyde (141 mg, 1.31 mmol), followed by the addition of a catalytic amount of acetic acid, after which the reaction was stirred at 65° C. until the imine formation was complete. Subsequently, sodium cyanoborohydride (206 mg, 3.27 mmol) was added after which the reaction was continued at room temperature for 4 h. After the completion of the reaction was confirmed by TLC and LC-MS, cold water was added to the reaction mixture and extraction with EtOAc was performed. The combined organic layers were washed with sat. brine, dried over anhydrous sodium sulfate and evaporated under reduced pressure. The obtained crude residue was purified by column chromatography over silica gel (2-3% MeOH in CH₂Cl₂) to afford 4-isobutyl-2-[3-(pyridazin-3-ylmethylamino)azetidin-1-yl]benzonitrile as a gummy liquid (200 mg, 57%).

Final tetrazole reaction was performed, by mixing the nitrile (50 mg, 0.156 mmol), sodium azide (81 mg, 1.24 mmol) and Bu₃SnCl (0.34 mL, 1.24 mmol) in toluene (5 mL) at 140° C. for 14 h in a sealed tube. Aq. work-up and subsequent column chromatography (4-5% MeOH in CH₂Cl₂) as described in method W, followed by trituration of the purified compound using diethyl ether, yielded the envisaged tetrazole A-236 as a colorless solid (6 mg, 11%).

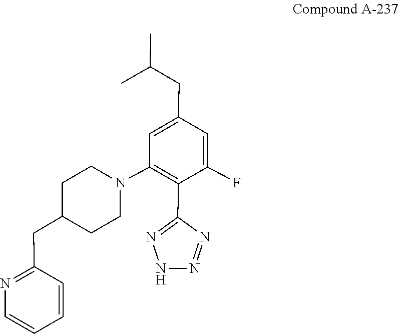 [SMILES: CC(C)Cc1cc(F)c(-c2nn[nH]n2)c(N2CCC(Cc3ccccn3)CC2)c1]

Prepared through a similar protocol as described in method AB. To a stirred solution of intermediate 58 (300 mg, 1.41 mmol) in DMF (2 mL) were added DIPEA (0.49 mL, 2.82 mmol) and K₂CO₃ (390 mg, 2.82 mmol) at 0° C. after which the reaction was stirred at r.t. for 10 minutes. Next, 2,6-difluoro-4-isobutylbenzonitrile (248 mg, 1.27 mmol) was added at r.t. and the reaction was continued at 60° C. for 12 h. After the completion of the reaction was confirmed by TLC, the mixture was poured into water and extraction with EtOAc was performed. The combined organic layers were washed with sat. brine, dried over anhydrous sodium sulfate and evaporated under reduced pressure. The obtained crude residue was purified by column chromatography over silica gel (10-25% EtOAc in hexane) to afford the desired 2-fluoro-4-isobutyl-6-[4-(2-pyridylmethyl)-1-piperidyl]benzonitrile as a white solid (362 mg, 73%).

Final tetrazole reaction was performed, by mixing the nitrile (80 mg, 0.228 mmol), sodium azide (118 mg, 1.82 mmol) and Bu₃SnCl (0.49 mL, 1.82 mmol) in toluene (10 mL) at 140° C. for 18 h in a sealed tube. Aq. work-up as described in method AB and subsequent preparative HPLC purification afforded the desired 2-[[1-[3-fluoro-5-isobutyl-2-(2H-tetrazol-5-yl)phenyl]-4-piperidyl]methyl]pyridine as a white solid (30 mg, 33%).

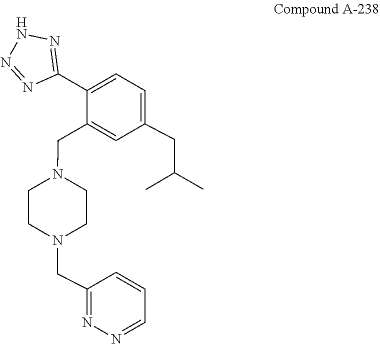 [SMILES: CC(C)Cc1ccc(-c2nn[nH]n2)c(CN2CCN(Cc3cccnn3)CC2)c1]

Prepared in a similar fashion as described in method D from intermediate 57 (450 mg, 1.53 mmol) and 3-(chloromethyl)pyridazine (295 mg, 2.30 mmol). The alkylation reaction was completed after 12 h at 65° C. After performing an aq. work-up as described in method D, the organic residue was purified by silica chromatography (2-3% MeOH in CH₂Cl₂) to afford 4-isobutyl-2-[[4-(pyridazin-3-ylmethyl)piperazin-1-yl]methyl]benzonitrile as a gummy liquid (200 mg, 37%).

Final tetrazole reaction was performed, by mixing the nitrile (150 mg, 0.429 mmol), sodium azide (223 mg, 3.43 mmol) and Bu₃SnCl (0.93 mL, 3.43 mmol) in toluene (10 mL) at 140° C. for 14 h in a sealed tube. Aq. work-up, followed by column chromatography (4-5% MeOH in CH₂Cl₂) and subsequent trituration of the purified compound with diethyl ether, yielded the envisaged tetrazole A-238 as an off-white solid (30 mg, 18%).

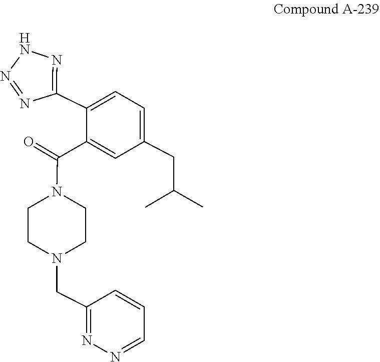 [SMILES: CC(C)Cc1ccc(-c2nn[nH]n2)c(C(=O)N2CCN(Cc3cccnn3)CC2)c1]

To a stirred solution of 3-(piperazin-1-ylmethyl)pyridazine hydrochloride (150 mg, 0.699 mmol) in DMF (5 mL) at 0° C. was added intermediate 56 (156 mg, 0.769 mmol), followed by EDC·HCl (161 mg, 0.838 mmol), HOBt (113 mg, 0.838 mmol) and DIPEA (0.30 mL, 1.75 mmol). Upon completion of the addition, the reaction mixture was allowed to stir at r.t. for 12 h until complete conversion was observed by TLC. Water was added to the reaction mixture and extraction with EtOAc was performed. The combined organic layers were washed with sat. brine, dried over anhydrous sodium sulfate and evaporated under reduced pressure. The crude residue was purified by silica chromatography (2-3% of MeOH in CH₂Cl₂) to afford 4-isobutyl-2-[4-(pyridazin-3-ylmethyl)piperazine-1-carbonyl]benzonitrile as a gummy liquid (104 mg, 41%).

Final tetrazole reaction was performed, by mixing the nitrile (65 mg, 0.179 mmol), NaN₃ (93 mg, 1.43 mmol) and Bu₃SnCl (0.39 mL, 1.43 mmol) in toluene (5 mL) at 140° C. for 14 h in a sealed tube. Aq. work-up, as described in method A, preparative HPLC purification and ultimate trituration of the purified compound with diethyl ether, yielded [5-isobutyl-2-(2H-tetrazol-5-yl)phenyl]-[4-(pyridazin-3-ylmethyl)piperazin-1-yl]methanone as a colorless solid (8 mg, 11%).

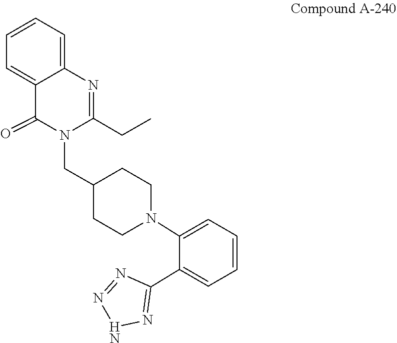 [SMILES: CCc1nc2ccccc2c(=O)n1CC1CCN(c2ccccc2-c2nn[nH]n2)CC1]

Prepared through method AE.

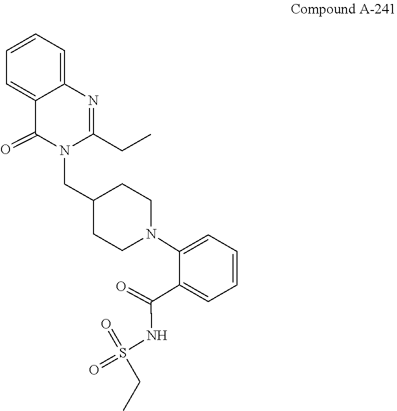 [SMILES: CCc1nc2ccccc2c(=O)n1CC1CCN(c2ccccc2C(=O)NS(=O)(=O)CC)CC1]

Prepared through method AE.

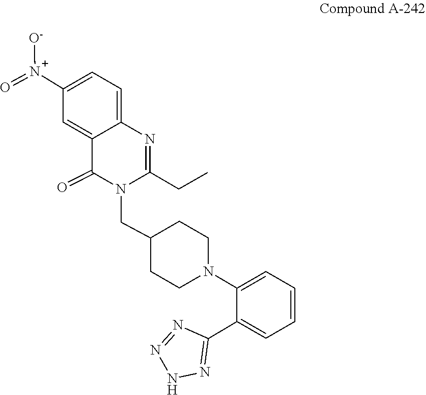 [SMILES: CCc1nc2ccc([N+](=O)[O-])cc2c(=O)n1CC1CCN(c2ccccc2-c2nn[nH]n2)CC1]

Prepared through method AF by mixing intermediate 59 (50 mg, 0.120 mmol), NaN₃ (62 mg, 0.958 mmol) and Bu₃SnCl (0.26 mL, 0.958 mmol) in xylene (10 mL) at 140° C. for 14 h in a sealed tube. Aq. work-up, as described in method AF, followed by column chromatography (SiO₂, 5-8% MeOH in CH₂Cl₂) and ultimate trituration of the purified compound using diethyl ether, yielded 2-ethyl-6-nitro-3-[[1-[2-(2H-tetrazol-5-yl)phenyl]-4-piperidyl]methyl]quinazolin-4-one as a pale-yellow solid (32 mg, 11%).

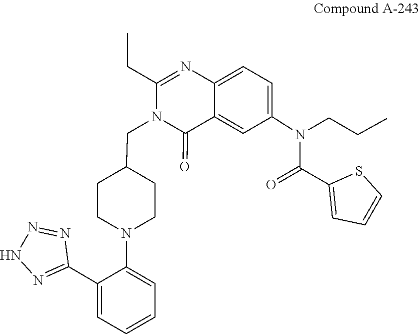 [SMILES: CCCN(C(=O)c1cccs1)c1ccc2nc(CC)n(CC3CCN(c4ccccc4-c4nn[nH]n4)CC3)c(=O)c2c1]

Prepared through method AF by adding thiophene-2-carbonyl chloride (82 mg, 0.557 mmol) to a solution of 2-[4-[(6-amino-2-ethyl-4-oxo-quinazolin-3-yl)methyl]-1-piperidyl]benzonitrile (180 mg, 0.465 mmol) and K₂CO₃ (128 mg, 0.929 mmol) in 1,4-dioxane (10 mL) at 0° C. The targeted amide was obtained after stirring the reaction mixture for 14 h at r.t. After performing an aq. work-up and column chromatography (34% of MeOH in CH₂Cl₂) N-[3-[[1-(2-cyanophenyl)-4-piperidyl]methyl]-2-ethyl-4-oxo-quinazolin-6-yl]thiophene-2-carboxamide was obtained as a pale-brown solid (139 mg, 60%).

Next, a tetrazole reaction was performed, by mixing the nitrile (100 mg, 0.201 mmol), NaN₃ (105 mg, 1.61 mmol) and Bu₃SnCl (0.44 mL, 1.61 mmol) in xylene (10 mL) at 140° C. for 14 h in a sealed tube. Aq. work-up as described in method AF, followed by column chromatography (SiO₂, 6-8% MeOH in CH₂Cl₂) and trituration in diethyl ether, afforded the desired tetrazole as a pale-yellow solid (32 mg, 29%).

To an ice-cold solution of N-[2-ethyl-4-oxo-3-[[1-[2-(2H-tetrazol-5-yl)phenyl]-4-piperidyl]methyl]quinazolin-6-yl]thiophene-2-carboxamide (1.00 g, 1.85 mmol) in anhydrous CH₂Cl₂ (25 mL) was added Et₃N (0.39 mL, 2.77 mmol), followed by careful addition of trityl chloride (0.50 mL, 2.03 mmol). Upon completion of the addition, the reaction was allowed to slowly warm up to room temperature and continued stirring at r.t. for an additional 3 hours. Aq. work-up as described in method AF, followed by trituration with diethyl ether yielded N-[2-ethyl-4-oxo-3-[[1-[2-(2-trityltetrazol-5-yl)phenyl]-4-piperidyl]methyl]quinazolin-6-yl]thiophene-2-carboxamide as an off-white solid (1.22 g, 84%).

To a stirred solution of N-[2-ethyl-4-oxo-3-[[1-[2-(2-trityltetrazol-5-yl)phenyl]-4-piperidyl]methyl]quinazolin-6-yl]thiophene-2-carboxamide (100 mg, 0.128 mmol) in DMF (5 mL) was added Cs₂CO₃ (83 mg, 0.255 mmol), followed by the addition of 1-chloropropane (0.014 mL, 0.153 mmol) and a catalytic amount of TBAB, after which the reaction was continued stirring at r.t. for 14 h. An aq. work-up and trituration with diethyl ether delivered the alkylated intermediate of interest (90 mg, crude).

Final trityl deprotection was performed, by adding HCl (g) in dioxane (5 mL) to a stirred solution of the alkylated intermediate (90 mg, 0.109 mmol) in 1,4-dioxane at 0° C., after which the reaction was left stirring at room temperature for 2 h. The reaction mixture was concentrated in vacuo, followed by a trituration with diethyl ether, affording the title compound A-243 as an off-white solid (34 mg, 46% over 2 steps).

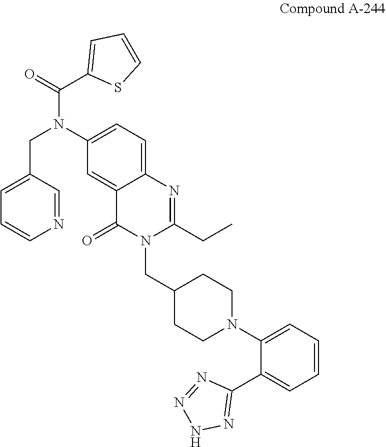 [SMILES: CCc1nc2ccc(N(Cc3cccnc3)C(=O)c3cccs3)cc2c(=O)n1CC1CCN(c2ccccc2-c2nn[nH]n2)CC1]

Prepared through method AF in a similar manner as compound A-243. To a stirred solution of N-[2-ethyl-4-oxo-3-[[1-[2-(2-trityltetrazol-5-yl)phenyl]-4-piperidyl]methyl]quinazolin-6-yl]thiophene-2-carboxamide (100 mg, 0.128 mmol) in DMF (5 mL) was added Cs₂CO₃ (83 mg, 0.255 mmol), followed by the addition of 3-(chloromethyl)pyridine (20 mg, 0.153 mmol) and a catalytic amount of TBAB, after which the reaction was continued stirring at r.t. for 14 h. An aq. work-up and trituration with diethyl ether delivered the targeted alkylated intermediate (90 mg, crude).

Final trityl deprotection was performed, by adding HCl (g) in dioxane (5 mL) to a stirred solution of the alkylated intermediate (90 mg, 0.103 mmol) in 1,4-dioxane at 0° C., after which the reaction was left stirring at room temperature for 2 h. Concentration of the reaction mixture in vacuo, followed by a trituration with diethyl ether, delivered N-[2-ethyl-4-oxo-3-[[1-[2-(2H-tetrazol-5-yl)phenyl]-4-piperidyl]methyl]quinazolin-6-yl]-N-(3-pyridylmethyl)thiophene-2-carboxamide as an off-white solid (39 mg, 48% over 2 steps).

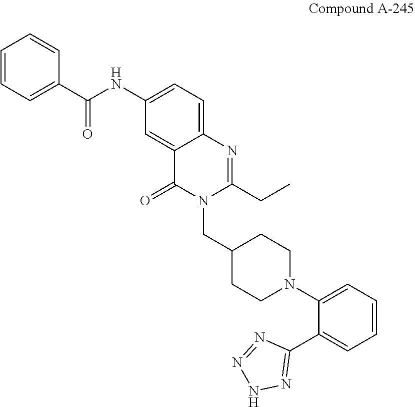 [SMILES: CCc1nc2ccc(NC(=O)c3ccccc3)cc2c(=O)n1CC1CCN(c2ccccc2-c2nn[nH]n2)CC1]

Prepared through method AF by adding benzoyl chloride (81 mg, 0.573 mmol) to a solution of 2-[4-[(6-amino-2-ethyl-4-oxo-quinazolin-3-yl)methyl]-1-piperidyl]benzonitrile (185 mg, 0.478 mmol) and K₂CO₃ (132 mg, 0.955 mmol) in 1,4-dioxane (10 mL) at 0° C. The envisaged compound was obtained after stirring the reaction mixture for 14 h at r.t. After performing an aq. work-up and column chromatography (34% of MeOH in CH₂Cl₂) N-[3-[[1-(2-cyanophenyl)-4-piperidyl]methyl]-2-ethyl-4-oxo-quinazolin-6-yl]benzamide was obtained as an off-white solid in good yield (162 mg, 69%).

Final tetrazole reaction was performed, by mixing the nitrile (100 mg, 0.203 mmol), NaN₃ (106 mg, 1.63 mmol) and Bu₃SnCl (0.45 mL, 1.63 mmol) in xylene (10 mL) at 140° C. for 14 h in a sealed tube. Aq. work-up as described in method AF, followed by column chromatography (SiO₂, 5-8% MeOH in CH₂Cl₂) and ultimate trituration in diethyl ether, afforded the desired final compound as a pale-yellow solid (22 mg, 20%).

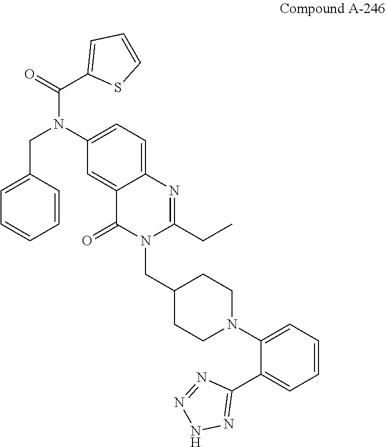 [SMILES: CCc1nc2ccc(N(Cc3ccccc3)C(=O)c3cccs3)cc2c(=O)n1CC1CCN(c2ccccc2-c2nn[nH]n2)CC1]

Prepared through method AF in a similar manner as compound A-243, by mixing N-[2-ethyl-4-oxo-3-[[1-[2-(2-trityltetrazol-5-yl)phenyl]-4-piperidyl]methyl]quinazolin-6-yl]thiophene-2-carboxamide (100 mg, 0.128 mmol) in DMF (5 mL) with Cs₂CO₃ (83 mg, 0.255 mmol) and benzyl chloride (0.018 mL, 0.153 mmol), in the presence of a catalytic amount of TBAB, after which the reaction was continued stirring at r.t. for 14 h. An aq. work-up and trituration with diethyl ether delivered the envisaged alkylated intermediate (90 mg, crude).

Final trityl deprotection was performed, by adding HCl (g) in dioxane (5 mL) to a stirred solution of the alkylated intermediate (90 mg, 0.103 mmol) in 1,4-dioxane at 0° C., after which the reaction was left stirring at room temperature for 2 h. Concentration of the reaction mixture under reduced pressure, followed by a trituration with diethyl ether, afforded the targeted title compound A-246 as an off-white solid (28 mg, 35% over 2 steps).

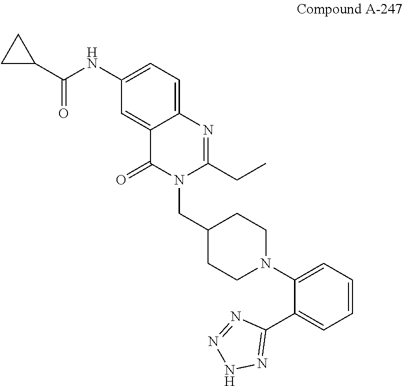 [SMILES: CCc1nc2ccc(NC(=O)C3CC3)cc2c(=O)n1CC1CCN(c2ccccc2-c2nn[nH]n2)CC1]

Prepared through method AF. To a solution of 2-[4-[(6-amino-2-ethyl-4-oxo-quinazolin-3-yl)methyl]-1-piperidyl]benzonitrile (50 mg, 0.129 mmol) in anhydrous DMF (3 mL) at 0° C. was added cyclopropanecarboxylic acid (13 mg, 0.142 mmol), followed by HATU (65 mg, 0.170 mmol) and DIPEA (0.062 mL, 0.355 mmol). The targeted amide was obtained after stirring the reaction mixture for 14 h at r.t. After performing an aq. work-up and column chromatography (3-5% of MeOH in CH₂Cl₂) N-[3-[[1-(2-cyanophenyl)-4-piperidyl]methyl]-2-ethyl-4-oxo-quinazolin-6-yl]cyclopropanecarboxamide was obtained as a pale-yellow solid (39 mg, 66%).

A final tetrazole reaction was performed, by mixing the nitrile (100 mg, 0.220 mmol), NaN₃ (114 mg, 1.76 mmol) and Bu₃SnCl (0.48 mL, 1.76 mmol) in xylene (10 mL) at 140° C. for 14 h in a sealed tube. Aq. work-up as described in method AF, followed by column chromatography (SiO₂, 6-8% MeOH in CH₂Cl₂) and trituration in diethyl ether, yielded the envisaged tetrazole A-247 as a pale-yellow solid (38 mg, 34%).

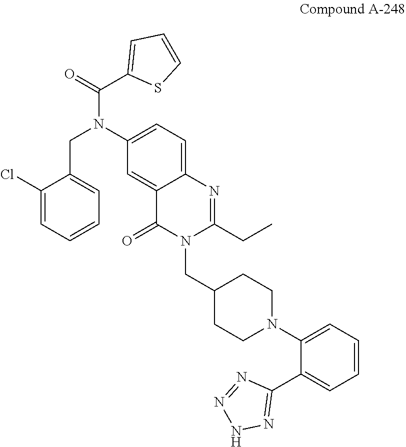 [SMILES: CCc1nc2ccc(N(Cc3ccccc3Cl)C(=O)c3cccs3)cc2c(=O)n1CC1CCN(c2ccccc2-c2nn[nH]n2)CC1]

Prepared through method AF in a similar manner as compound A-243. To a stirred solution of N-[2-ethyl-4-oxo-3-[[1-[2-(2-trityltetrazol-5-yl)phenyl]-4-piperidyl]methyl]quinazolin-6-yl]thiophene-2-carboxamide (100 mg, 0.128 mmol) in DMF (5 mL) was added Cs₂CO₃ (83 mg, 0.255 mmol), followed by the addition of 1-chloro-2-(chloromethyl)benzene (25 mg, 0.153 mmol) and a catalytic amount of TBAB, after which the reaction was continued stirring at r.t. for 14 h. An aq. work-up as described in method AF and trituration with diethyl ether delivered the targeted alkylated intermediate (100 mg, crude).

Final trityl deprotection was performed, by adding HCl (g) in dioxane (5 mL) to a stirred solution of the alkylated intermediate (100 mg, 0.110 mmol) in 1,4-dioxane at 0° C., after which the reaction was left stirring at room temperature for 2 h. Concentration of the reaction mixture in vacuo, followed by a trituration with diethyl ether, delivered N-[(2-chlorophenyl)methyl]-N-[2-ethyl-4-oxo-3-[[1-[2-(2H-tetrazol-5-yl)phenyl]-4-piperidyl]methyl]quinazolin-6-yl]thiophene-2-carboxamide as an off-white solid (36 mg, 42% over 2 steps).

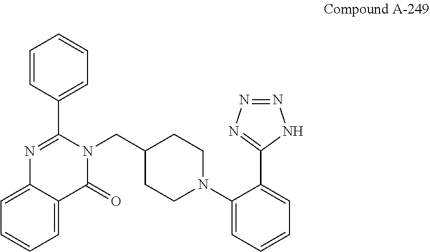 [SMILES: O=c1c2ccccc2nc(-c2ccccc2)n1CC1CCN(c2ccccc2-c2nnn[nH]2)CC1]

Prepared through method AG.

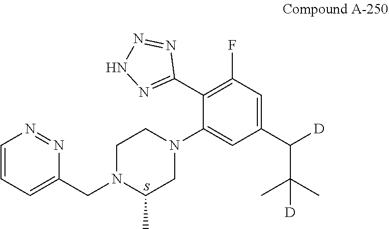

Compound 250 (a deuterated analog of compound 174) was prepared through method G from intermediate 60. To a stirred solution of N-Boc protected (S)-methyl piperazine (30 g, 150 mmol) in DMF (400 mL) was added DIPEA (78.3 mL, 449 mmol) at 0° C., after which the reaction was stirred at r.t. for 10 minutes. Then 3-(chloromethyl)pyridazine (23.1 g, 180 mmol) was added and the reaction was continued at 60° C. for 14 h. After completion of the reaction was confirmed by TLC, the reaction mixture was diluted with cold water and extracted with CH₂Cl₂. The combined organic layers were washed with sat. brine, dried over anhydrous sodium sulfate and evaporated under reduced pressure. The obtained crude residue was purified by column chromatography over silica gel (eluting with 2-3% MeOH in CH₂Cl₂) to afford (S)-tert-butyl 3-methyl-4-(pyridazin-3-ylmethyl)piperazine-1-carboxylate (26.3 g, 60%).

To the stirred solution of (S)-tert-butyl 3-methyl-4-(pyridazin-3-ylmethyl)piperazine-1-carboxylate (1.0 g, 3.42 mmol) in 1,4-dioxane (5 mL), HCl (g) in dioxane (10 mL) was added at 0° C. and the reaction was continued at r.t. for 5 h. After the completion of the reaction was confirmed by TLC, the reaction mixture was evaporated to dryness under reduced pressure, then washed with hexane to afford a crude residue (S)-3-((2-methylpiperazin-1-yl)methyl)pyridazine hydrochloride. The crude compound thus obtained was taken to the next step without purification (750 mg crude).

To a stirred solution of (S)-3-((2-methylpiperazin-1-yl)methyl)pyridazine hydrochloride (740 mg, 3.24 mmol) in DMF (10 mL) were added DIPEA (1.41 mL, 8.09 mmol) and K₂CO₃ (894 mg, 6.47 mmol) at 0° C. and the reaction was stirred at r.t. for 10 minutes. Then intermediate 60 (702 mg, 3.56 mmol) was added at r.t. and the reaction was continued at 65° C. for 14 h. After completion of the reaction was confirmed by TLC, the reaction mixture was diluted with cold water and extraction with CH₂Cl₂ was performed. The combined organic layers were washed with sat. brine, dried over anhydrous sodium sulfate and evaporated under reduced pressure. The crude thus obtained was purified by column chromatography over silica gel eluting with 4-5% MeOH in CH₂Cl₂ to afford the envisaged SNAr product (708 mg, 56% over 2 steps).

A mixture of this isolated nitrile (400 mg, 1.08 mmol), NaN₃ (563 mg, 8.66 mmol) and Bu₃SnCl (2.35 mL, 8.66 mmol) in toluene (10 mL) was stirred at 143° C. for 18 h in a sealed tube. After the completion of the reaction was confirmed by TLC, the reaction mixture was evaporated under reduced pressure. The residue was dissolved in CH₂Cl₂ and washed with a 10% NaOH solution. The aqueous layer was then neutralized with a citric acid solution and extracted with CH₂Cl₂ (3×20 mL). The combined organic layers were washed with sat. brine, dried over anhydrous sodium sulfate and evaporated under reduced pressure. The crude compound thus obtained was purified by silica chromatography (6-8% of MeOH in CH₂Cl₂) to afford 3-[[(2S)-4-[5-(1,2-dideuterio-2-methyl-propyl)-3-fluoro-2-(2H-tetrazol-5-yl)phenyl]-2-methyl-piperazin-1-yl]methyl]pyridazine which was triturated with diethyl ether to obtain an off-white solid (80 mg, 18%).

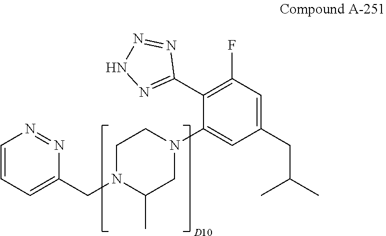

Compound 251 (another deuterated analog of compound 174) was prepared in a similar fashion as described in method E. To a stirred solution of intermediate 10 (500 mg, 2.56 mmol) in DMF (5 mL) were added 2-methylpiperazine-d10 (311 mg, 2.82 mmol) and K₂CO₃ (1.06 g, 7.68 mmol) at r.t., after which the reaction was continued at 100° C. for 8 h. After completion of the reaction was confirmed by TLC, an aq. work-up was performed as described in method E. The obtained crude residue was purified by preparative HPLC to yield the envisaged SNAr product as an off-white solid (501 mg, 69%).

To a stirred solution of 3-(chloromethyl)pyridazine (585 mg, 4.55 mmol) in CH₃CN (15 mL) at 20° C. was added K₃PO₄ (2.41 g, 11.38 mmol), after which the reaction mixture was kept stirring at this temperature for 30 min. A catalytic amount of KI (151 mg, 0.91 mmol) and 2-fluoro-6-[2,2,3,3,5,6,6-heptadeuterio-5-(trideuteriomethyl) piperazin-1-yl]-4-isobutyl-benzonitrile (451 mg, 4.10 mmol) were added, after which the reaction was stirred at r.t. for an additional 24 hours. After completion of the reaction was confirmed by TLC, the reaction mixture was diluted with cold water and extraction with EtOAc was performed. The combined organic layers were washed with sat. brine, dried over anhydrous sodium sulfate and evaporated under reduced pressure. The crude thus obtained was purified by preparative HPLC to afford the desired product 2-fluoro-6-[2,2,3,3,5,6,6-heptadeuterio-4-(pyridazin-3-ylmethyl)-5-(trideuteriomethyl)piperazin-1-yl]-4-isobutyl-benzonitrile (310 mg, 20%) as an off-white solid.

A mixture of this isolated nitrile (250 mg, 0.66 mmol), NaN₃ (344 mg, 5.30 mmol) and Bu₃SnCl (1.44 mL, 5.30 mmol) in toluene (5 mL) was stirred at 148° C. for 48 h in a sealed tube. After the completion of the reaction was confirmed by TLC, the reaction mixture was evaporated under reduced pressure. The residue was re-dissolved in CH₂Cl₂ and washed with a 10% NaOH solution. The aqueous layer was then neutralized with a citric acid solution and extracted with CH₂Cl₂. The combined organic layers were washed with sat. brine, dried over anhydrous sodium sulfate and evaporated under reduced pressure. The crude compound thus obtained was purified by preparative HPLC to afford the targeted substituted tetrazole which was additionally triturated with diethyl ether to obtain an off-white solid (78 mg, 28%).

Table 1 below (compounds numbered A-01 to A-251) give the chemical structure, name and molecular weight (both calculated and as determined using mass spectrometry) of some of the preferred but non-limiting compounds of the invention. The NMR data for each of the compounds A-01 to A-251 in Table 1 is given in Table 2 below. Compounds mentioned in the Tables below for which a detailed description of their synthesis is not given in the Experimental Part herein, as well as other compounds of the invention not specifically described herein, can be synthesized using one or more of the synthetic routes and techniques described herein, for example in a manner analogous to the synthesis of comparable compounds for which suitable synthetic routes and techniques are given herein.

TABLE 1

| Compound | | | | | MS |
|---|---|---|---|---|---|
| number | Structure | Name | Method | MW | Ms′d |
| A-01 | 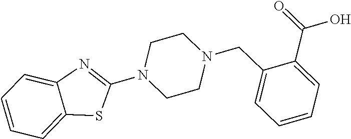 [SMILES: O=C(O)c1ccccc1CN1CCN(c2nc3ccccc3s2)CC1] | 2-[[4-(1,3-benzo- thiazol-2-yl)piper- azin-1-yl]methyl]- benzoic acid | H | 353.44 | 354.1 |
| A-02 | 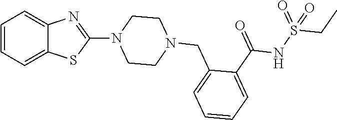 [SMILES: CCS(=O)(=O)NC(=O)c1ccccc1CN1CCN(c2nc3ccccc3s2)CC1] | 2-[[4-(1,3-benzo- thiazol-2-yl)piper- azin-1-yl]methyl]- N-ethylsulfonyl- benzamide | H | 444.57 | 445.1 |
| A-03 | 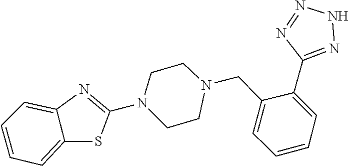 [SMILES: c1ccc(-c2nn[nH]n2)c(CN2CCN(c3nc4ccccc4s3)CC2)c1] | 2-[4-[[2-(2H- tetrazol-5-yl)phen- yl]-methyl]piper- azin-1-yl]-1,3- benzothiazole | H | 377.47 | 378.2 |
| A-04 | 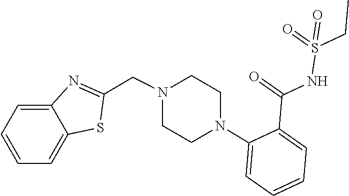 [SMILES: CCS(=O)(=O)NC(=O)c1ccccc1N1CCN(Cc2nc3ccccc3s2)CC1] | 2-[4-(1,3-benzo- thiazol-2-ylmeth- yl)piperazin-1-yl]- N-ethylsulfonyl- benzamide | I | 444.57 | 445.1 |
| A-05 | 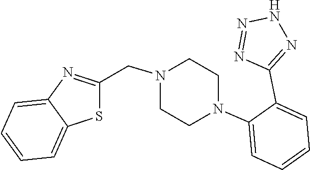 [SMILES: c1ccc(N2CCN(Cc3nc4ccccc4s3)CC2)c(-c2nn[nH]n2)c1] | 2-[[4-[2-(2H- tetrazol-5-yl)phen- yl]piperazin-1-yl]- methyl]-1,3-benzo- thiazole | I | 377.47 | 378.2 |
| A-06 | 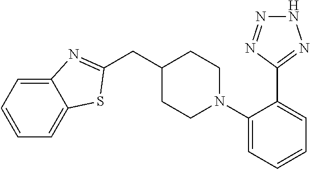 [SMILES: c1ccc(N2CCC(Cc3nc4ccccc4s3)CC2)c(-c2nn[nH]n2)c1] | 2-[[1-[2-(2H- tetrazol-5-yl)phen- yl]-4-piperidyl]- methyl]-1,3-benzo- thiazole | J | 376.48 | 377.1 |
| A-07 | 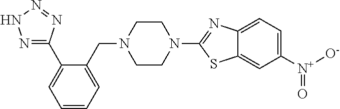 [SMILES: O=[N+]([O-])c1ccc2nc(N3CCN(Cc4ccccc4-c4nn[nH]n4)CC3)sc2c1] | 6-nitro-2-[4-[[2- (2H-tetrazol-5-yl)- phenyl]methyl]- piperazin-1-yl]-1,3- benzothiazole | H | 422.46 | 423.2 |
| A-08 | 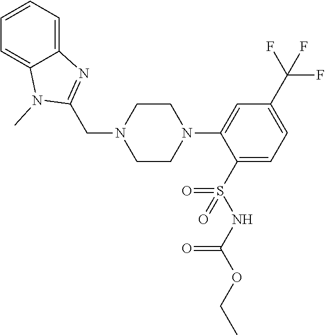 [SMILES: CCOC(=O)NS(=O)(=O)c1ccc(C(F)(F)F)cc1N1CCN(Cc2nc3ccccc3n2C)CC1] | ethyl N-[2-[4-[(1- methylbenzimid- azol-2-yl)methyl]- piperazin-1-yl]-4- (trifluoromethyl)- phenyl]sulfonyl- carbamate | K | 525.54 | 526.3 |
| A-09 | 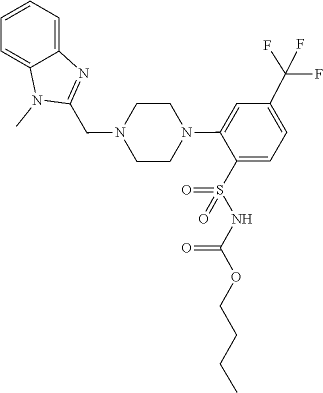 [SMILES: CCCCOC(=O)NS(=O)(=O)c1ccc(C(F)(F)F)cc1N1CCN(Cc2nc3ccccc3n2C)CC1] | butyl N-[2-[4-[(1- methylbenzimid- azol-2-yl)methyl]- piperazin-1-yl]-4- (trifluoromethyl)- phenyl]sulfonyl- carbamate | K | 553.60 | 554.3 |
| A-10 | 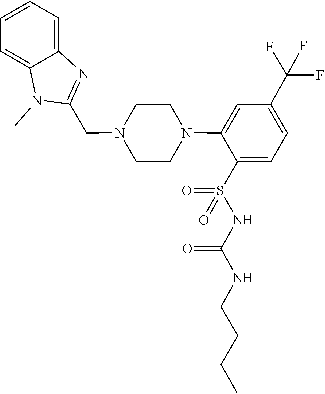 [SMILES: CCCCNC(=O)NS(=O)(=O)c1ccc(C(F)(F)F)cc1N1CCN(Cc2nc3ccccc3n2C)CC1] | 1-butyl-3-[2-[4-[(1- methylbenzimid- azol-2-yl)methyl]- piperazin-1-yl]-4- (trifluoromethyl)- phenyl]sulfonyl- urea | K | 552.61 | 551.3 552.3 |
| A-11 | 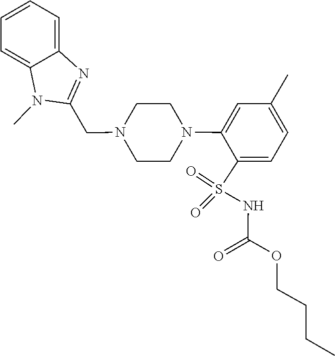 | butyl N-[4-methyl- 2-[4-[(1-methyl- benzimidazol-2-yl)- methyl]piperazin- 1-yl]phenyl]sulfon- ylcarbamate | K | 499.63 | 500.3 |
| A-12 | 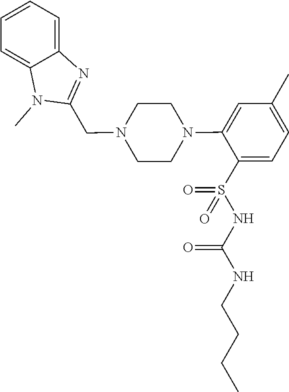 [SMILES: CCCCNC(=O)NS(=O)(=O)c1ccc(C)cc1N1CCN(Cc2nc3ccccc3n2C)CC1] | 1-butyl-3-[4-meth- yl-2-[4-[(1-methyl- benzimidazol-2-yl)- methyl]piperazin-1- yl]phenyl]sulfonyl- urea | K | 498.64 | 499.4 |
| A-13 | 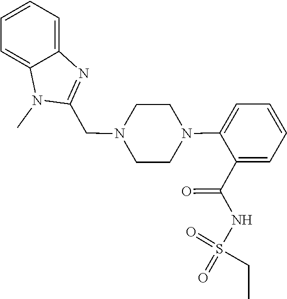 [SMILES: CCS(=O)(=O)NC(=O)c1ccccc1N1CCN(Cc2nc3ccccc3n2C)CC1] | N-ethylsulfonyl-2- [4-[(1-methylbenz- imidazol-2-yl)meth- yl]piperazin-1-yl]- benzamide | N | 441.55 | 442.2 |
| A-14 | 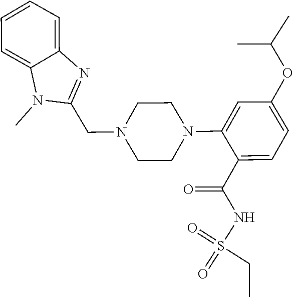 [SMILES: CCS(=O)(=O)NC(=O)c1ccc(OC(C)C)cc1N1CCN(Cc2nc3ccccc3n2C)CC1] | N-ethylsulfonyl-4- isopropoxy-2-[4- [(1-methylbenz- imidazol-2-yl)meth- yl]piperazin-1-yl]- benzamide | O | 499.63 | 500.3 |
| A-15 | 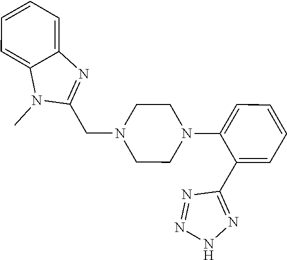 [SMILES: Cn1c(CN2CCN(c3ccccc3-c3nn[nH]n3)CC2)nc2ccccc21] | 1-methyl-2-[4-[2- (2H-tetrazol-5-yl)- phenyl]piperazin- 1-yl]methyl]benz- imidazole | L | 374.44 | 375.3 |
| A-16 | 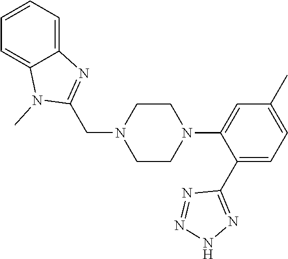 [SMILES: Cc1ccc(-c2nn[nH]n2)c(N2CCN(Cc3nc4ccccc4n3C)CC2)c1] | 1-methyl-2-[[4-[5- methyl-2-(2H- tetrazol-5-yl)phen- yl]piperazin-1-yl]- methyl]benzimid- azole | L | 388.47 | 389.3 |
| A-17 | 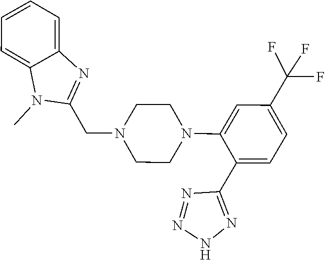 [SMILES: Cn1c(CN2CCN(c3cc(C(F)(F)F)ccc3-c3nn[nH]n3)CC2)nc2ccccc21] | 1-methyl-2-[[4-[2- (2H-tetrazol-5-yl)- 5-(trifluoromethyl)- phenyl]piperazin-1- yl]methyl]benzimid- azole | L | 442.44 | 443.3 |
| A-18 | 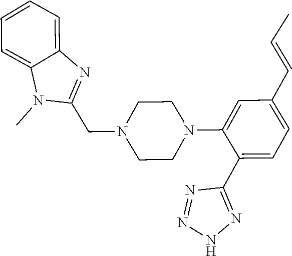 [SMILES: C/C=C/c1ccc(-c2nn[nH]n2)c(N2CCN(Cc3nc4ccccc4n3C)CC2)c1] | 1-methyl-2-[[4-[5- [(E)-prop-1-enyl]-2- (2H-tetrazol-5-yl)- phenyl]piperazin- 1-yl]methyl]benz- imidazole | L | 414.51 | 415.3 |
| A-19 | 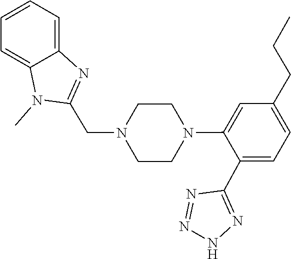 [SMILES: CCCc1ccc(-c2nn[nH]n2)c(N2CCN(Cc3nc4ccccc4n3C)CC2)c1] | 1-methyl-2-[[4-[5- propyl-2-(2H- tetrazol-5-yl)phen- yl]piperazin-1-yl]- methyl]benzimid- azole | L | 416.52 | 417.3 |
| A-20 | 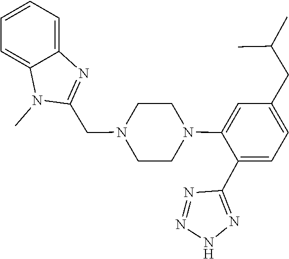 [SMILES: CC(C)Cc1ccc(-c2nn[nH]n2)c(N2CCN(Cc3nc4ccccc4n3C)CC2)c1] | 2-[[4-[5-isobutyl-2- (2H-tetrazol-5-yl)- phenyl]piperazin-1- yl]methyl]-1-meth- yl-benzimidazole | L | 430.55 | 431.4 |
| A-21 | 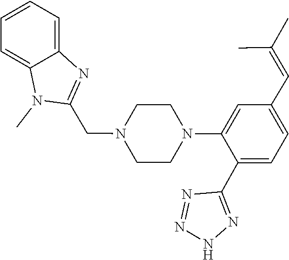 [SMILES: CC(C)=Cc1ccc(-c2nn[nH]n2)c(N2CCN(Cc3nc4ccccc4n3C)CC2)c1] | 1-methyl-2-[[4-[5- (2-methylprop-1- enyl)-2-(2H- tetrazol-5-yl)phen- yl]piperazin-1-yl]- methyl]benzimid- azole | L | 428.53 | 429.3 |
| A-22 | 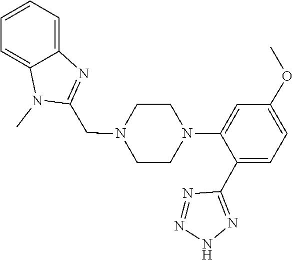 [SMILES: COc1ccc(-c2nn[nH]n2)c(N2CCN(Cc3nc4ccccc4n3C)CC2)c1] | 2-[[4-[5-methoxy-2- (2H-tetrazol-5-yl)- phenyl]piperazin-1- yl]methyl]-1-meth- yl-benzimidazole | L | 404.47 | 405.3 |
| A-23 | 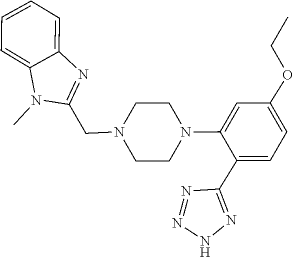 [SMILES: CCOc1ccc(-c2nn[nH]n2)c(N2CCN(Cc3nc4ccccc4n3C)CC2)c1] | 2-[[4-[5-ethoxy-2- (2H-tetrazol-5-yl)- phenyl]piperazin-1- yl]methyl]-1-meth- yl-benzimidazole | L | 418.49 | 419.3 |
| A-24 | 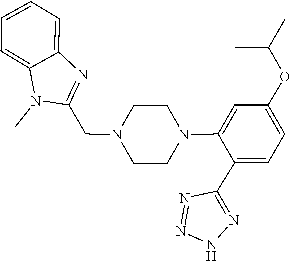 [SMILES: CC(C)Oc1ccc(-c2nn[nH]n2)c(N2CCN(Cc3nc4ccccc4n3C)CC2)c1] | 2-[4-[5-isopropoxy- 2-(2H-tetrazol-5- yl)phenyl]piperazin- 1-yl]methyl]-1- methyl-benzimid- azole | L | 432.52 | 433.3 |
| A-25 | 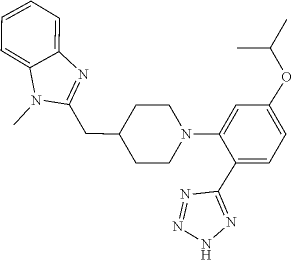 [SMILES: CC(C)Oc1ccc(-c2nn[nH]n2)c(N2CCC(Cc3nc4ccccc4n3C)CC2)c1] | 2-[[1-[5-isopropoxy- 2-(2H-tetrazol-5- yl)phenyl]-4- piperidyl]methyl]- 1-methyl-benzimid- azole | P | 431.53 | 432.3 |
| A-26 | 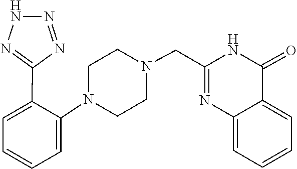 [SMILES: O=c1[nH]c(CN2CCN(c3ccccc3-c3nn[nH]n3)CC2)nc2ccccc12] | 2-[[4-[2-(2H- tetrazol-5-yl)phen- yl]piperazin-1-yl]- methyl]-3H-quin- azolin-4-one | M | 388.43 | 389.2 |
| A-27 | 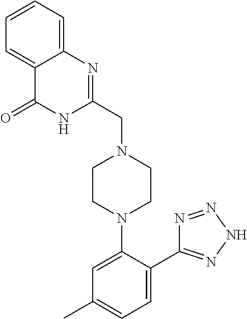 [SMILES: Cc1ccc(-c2nn[nH]n2)c(N2CCN(Cc3nc4ccccc4c(=O)[nH]3)CC2)c1] | 2-[[4-[5-methyl-2- (2H-tetrazol-5-yl)- phenyl]piperazin- 1-yl]methyl]-3H- quinazolin-4-one | M | 402.45 | 403.3 |
| A-28 | 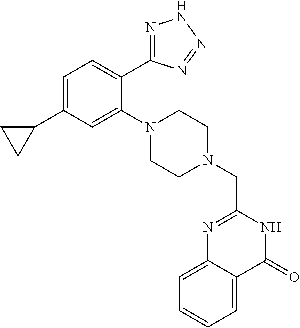 [SMILES: O=c1[nH]c(CN2CCN(c3cc(C4CC4)ccc3-c3nn[nH]n3)CC2)nc2ccccc12] | 2-[[4-[5-cycloprop- yl-2-(2H-tetrazol-5- yl)phenyl]piperazin- 1-yl]methyl]-3H- quinazolin-4-one | M | 428.49 | 429.2 |
| A-29 | 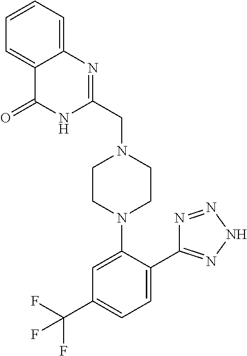 [SMILES: O=c1[nH]c(CN2CCN(c3cc(C(F)(F)F)ccc3-c3nn[nH]n3)CC2)nc2ccccc12] | 2-[[4-[2-(2H- tetrazol-5-yl)-5- (trifluoromethyl)- phenyl]piperazin- 1-yl]methyl]-3H- quinazolin-4-one | M | 456.42 | 457.2 |
| A-30 | 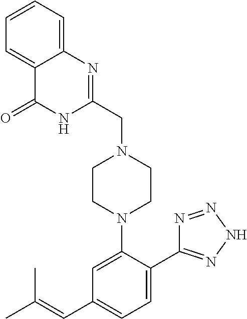 [SMILES: CC(C)=Cc1ccc(-c2nn[nH]n2)c(N2CCN(Cc3nc4ccccc4c(=O)[nH]3)CC2)c1] | 2-[[4-[5-(2-methyl- prop-1-enyl)-2- (2H-tetrazol-5-yl)- phenyl]piperazin- 1-yl]methyl]-3H- quinazolin-4-one | M | 442.52 | 443.3 |
| A-31 | 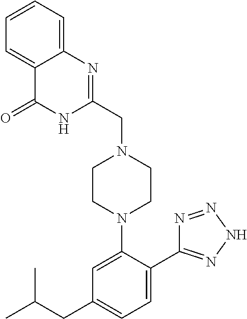 [SMILES: CC(C)Cc1ccc(-c2nn[nH]n2)c(N2CCN(Cc3nc4ccccc4c(=O)[nH]3)CC2)c1] | 2-[[4-[5-isobutyl-2- (2H-tetrazol-5-yl)- phenyl]piperazin- 1-yl]methyl]-3H- quinazolin-4-one | M | 444.53 | 445.3 |
| A-32 | 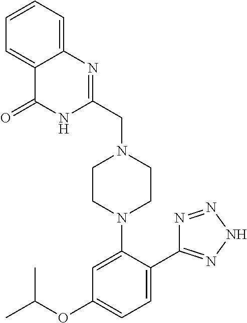 [SMILES: CC(C)Oc1ccc(-c2nn[nH]n2)c(N2CCN(Cc3nc4ccccc4c(=O)[nH]3)CC2)c1] | 2-[4-[5-isopropoxy- 2-(2H-tetrazol-5- yl)phenyl]piperazin- 1-yl]methyl]-3H- quinazolin-4-one | M | 446.50 | 447.3 |
| A-33 | 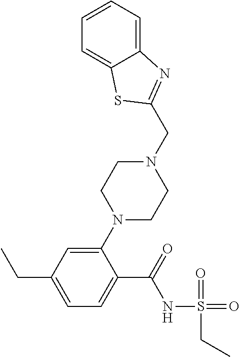 [SMILES: CCc1ccc(C(=O)NS(=O)(=O)CC)c(N2CCN(Cc3nc4ccccc4s3)CC2)c1] | 2-[4-(1,3-benzo- thiazol-2-ylmethyl)- piperazin-1-yl]-4- ethyl-N-ethylsulfon- yl-benzamide | B | 472.62 | 473.2 |
| A-34 | 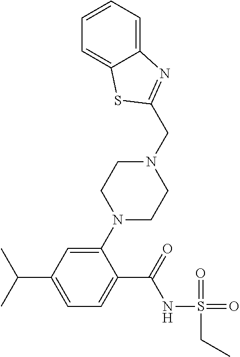 [SMILES: CCS(=O)(=O)NC(=O)c1ccc(C(C)C)cc1N1CCN(Cc2nc3ccccc3s2)CC1] | 2-[4-(1,3-benzo- thiazol-2-ylmethyl)- piperazin-1-yl]-N- ethylsulfonyl-4-iso- propyl-benzamide | B | 486.65 | 487.3 |
| A-35 | 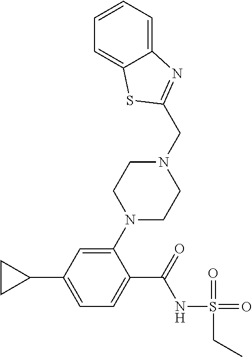 [SMILES: CCS(=O)(=O)NC(=O)c1ccc(C2CC2)cc1N1CCN(Cc2nc3ccccc3s2)CC1] | 2-[4-(1,3-benzo- thiazol-2-ylmethyl)- piperazin-1-yl]-4- cyclopropyl-N-eth- ylsulfonyl-benz- amide | B | 484.63 | 485.2 |
| A-36 | 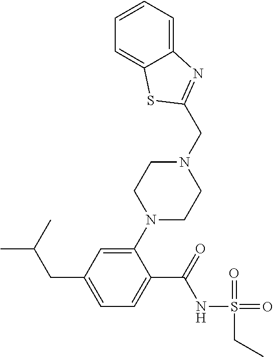 [SMILES: CCS(=O)(=O)NC(=O)c1ccc(CC(C)C)cc1N1CCN(Cc2nc3ccccc3s2)CC1] | 2-[4-(1,3-benzo- thiazol-2-ylmethyl)- piperazin-1-yl]-N- ethylsulfonyl-4- isobutyl-benzamide | B | 500.68 | 501.3 |
| A-37 | 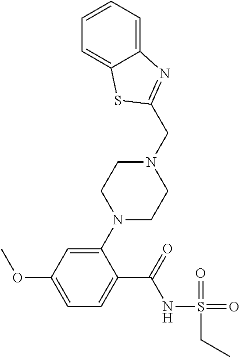 [SMILES: CCS(=O)(=O)NC(=O)c1ccc(OC)cc1N1CCN(Cc2nc3ccccc3s2)CC1] | 2-[4-(1,3-benzo- thiazol-2-ylmethyl)- piperazin-1-yl]-N- ethylsulfonyl-4- methoxy-benzamide | S | 474.60 | 475.2 |
| A-38 | 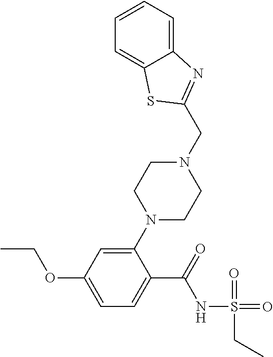 [SMILES: CCOc1ccc(C(=O)NS(=O)(=O)CC)c(N2CCN(Cc3nc4ccccc4s3)CC2)c1] | 2-[4-(1,3-benzo- thiazol-2-ylmethyl)- piperazin-1-yl]-4- ethoxy-N-ethyl- sulfonyl-benzamide | S | 488.62 | 489.2 |
| A-39 | 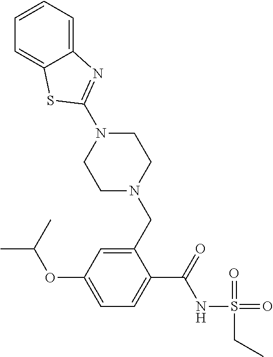 [SMILES: CCS(=O)(=O)NC(=O)c1ccc(OC(C)C)cc1CN1CCN(c2nc3ccccc3s2)CC1] | 2-[[4-(1,3-benzo- thiazol-2-yl)piper- azin-1-yl]methyl]- N-ethylsulfonyl-4- isopropoxy-benz- amide | R | 502.65 | 503.3 |
| A-40 | 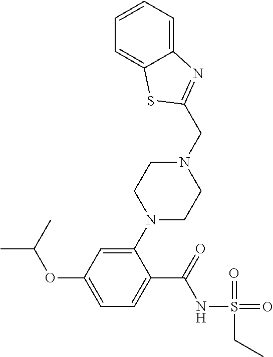 [SMILES: CCS(=O)(=O)NC(=O)c1ccc(OC(C)C)cc1N1CCN(Cc2nc3ccccc3s2)CC1] | 2-[4-(1,3-benzo- thiazol-2-ylmethyl)- piperazin-1-yl]-N- ethylsulfonyl-4- isopropoxy-benz- amide | Q | 502.65 | 503.3 |
| A-41 | 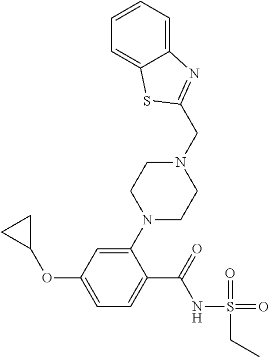 [SMILES: CCS(=O)(=O)NC(=O)c1ccc(OC2CC2)cc1N1CCN(Cc2nc3ccccc3s2)CC1] | 2-[4-(1,3-benzo- thiazol-2-ylmethyl)- piperazin-1-yl]-4- (cyclopropoxy)-N- ethylsulfonyl-benz- amide | S | 500.63 | 501.2 |
| A-42 | 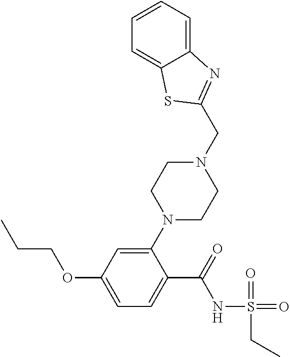 [SMILES: CCCOc1ccc(C(=O)NS(=O)(=O)CC)c(N2CCN(Cc3nc4ccccc4s3)CC2)c1] | 2-[4-(1,3-benzo- thiazol-2-ylmeth- yl)piperazin-1-yl]- N-ethylsulfonyl-4- propoxy-benzamide | Q | 502.65 | 503.2 |
| A-43 | 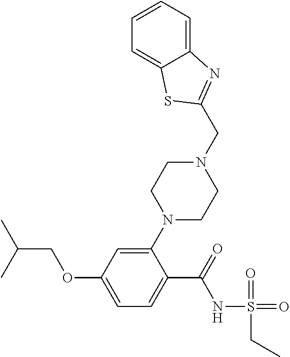 [SMILES: CCS(=O)(=O)NC(=O)c1ccc(OCC(C)C)cc1N1CCN(Cc2nc3ccccc3s2)CC1] | 2-[4-(1,3-benzo- thiazol-2-ylmeth- yl)piperazin-1-yl]- N-ethylsulfonyl-4- isobutoxy-benz- amide | S | 516.68 | 517.2 |
| A-44 | 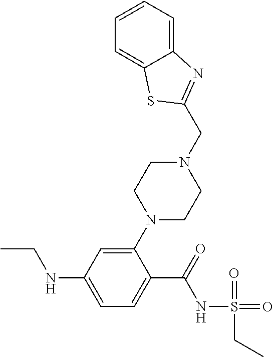 [SMILES: CCNc1ccc(C(=O)NS(=O)(=O)CC)c(N2CCN(Cc3nc4ccccc4s3)CC2)c1] | 2-[4-(1,3-benzo- thiazol-2-ylmethyl)- piperazin-1-yl]-4- (ethylamino)-N- ethylsulfonyl-benz- amide | T | 487.64 | 488.3 |
| A-45 | 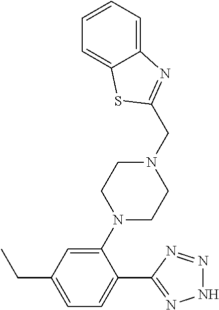 [SMILES: CCc1ccc(-c2nn[nH]n2)c(N2CCN(Cc3nc4ccccc4s3)CC2)c1] | 2-[[4-[5-ethyl-2- (2H-tetrazol-5-yl)- phenyl]piperazin- 1-yl]methyl]-1,3- benzothiazole | Y | 405.52 | 406.2 |
| A-46 | 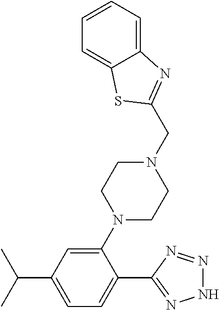 [SMILES: CC(C)c1ccc(-c2nn[nH]n2)c(N2CCN(Cc3nc4ccccc4s3)CC2)c1] | 2-[4-[5-isopropyl- 2-(2H-tetrazol-5- yl)phenyl]piperazin- 1-yl]methyl]-1,3- benzothiazole | Y | 419.55 | 420.3 |
| A-47 | 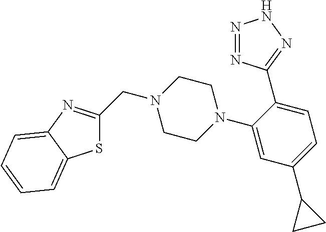 [SMILES: c1ccc2sc(CN3CCN(c4cc(C5CC5)ccc4-c4nn[nH]n4)CC3)nc2c1] | 2-[[4-[5-cycloprop- yl-2-(2H-tetrazol-5- yl)phenyl]piperazin- 1-yl]methyl]-1,3- benzothiazole | Y | 417.53 | 418.2 |
| A-48 | 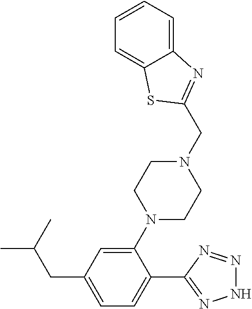 [SMILES: CC(C)Cc1ccc(-c2nn[nH]n2)c(N2CCN(Cc3nc4ccccc4s3)CC2)c1] | 2-[[4-[5-isobutyl-2- (2H-tetrazol-5-yl)- phenyl]piperazin- 1-yl]methyl]-1,3- benzothiazole | U | 433.57 | 434.3 |
| A-49 | 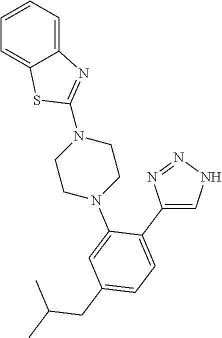 [SMILES: CC(C)Cc1ccc(-c2c[nH]nn2)c(N2CCN(c3nc4ccccc4s3)CC2)c1] | 2-[4-[5-isobutyl-2- (2H-tetrazol-5-yl)- phenyl]piperazin- 1-yl]-1,3-benzo- thiazole | U | 419.55 | 420.3 |
| A-50 | 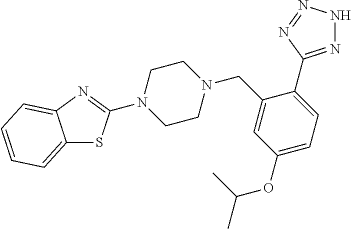 [SMILES: CC(C)Oc1ccc(-c2nn[nH]n2)c(CN2CCN(c3nc4ccccc4s3)CC2)c1] | 2-[4-[[5-isoprop- oxy-2-(2H-tetrazol- 5-yl)phenyl]meth- yl]piperazin-1-yl]- 1,3-benzothiazole | H | 435.55 | 436.2 |
| A-51 | 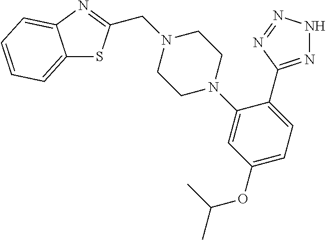 [SMILES: CC(C)Oc1ccc(-c2nn[nH]n2)c(N2CCN(Cc3nc4ccccc4s3)CC2)c1] | 2-[[4-[5-isoprop- oxy-2-(2H-tetrazol- 5-yl)phenyl]piper- azin-1-yl]methyl]- 1,3-benzothiazole | C | 435.55 | 436.2 |
| A-52 | 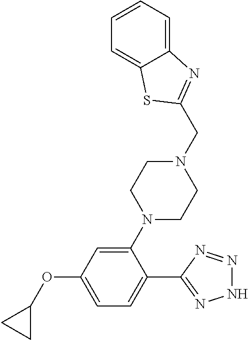 [SMILES: c1ccc2sc(CN3CCN(c4cc(OC5CC5)ccc4-c4nn[nH]n4)CC3)nc2c1] | 2-[[4-[5-(cyclo- propoxy)-2-(2H- tetrazol-5-yl)phen- yl]piperazin-1-yl]- methyl]-1,3-benzo- thiazole | Z | 433.53 | 434.3 |
| A-53 | 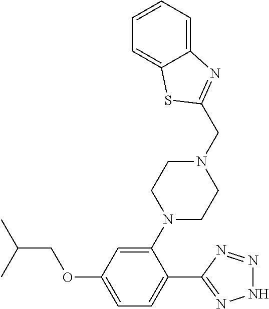 [SMILES: CC(C)COc1ccc(-c2nn[nH]n2)c(N2CCN(Cc3nc4ccccc4s3)CC2)c1] | 2-[[4-[5-isobutoxy- 2-(2H-tetrazol-5- yl)phenyl]piperazin- 1-yl]methyl]-1,3- benzothiazole | Z | 449.57 | 450.3 |
| A-54 | 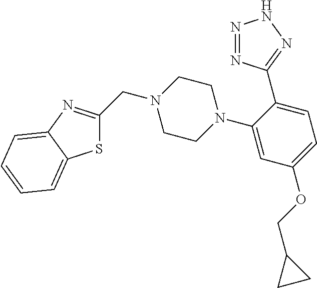 [SMILES: c1ccc2sc(CN3CCN(c4cc(OCC5CC5)ccc4-c4nn[nH]n4)CC3)nc2c1] | 2-[[4-[5-(cyclo- propylmethoxy)-2- (2H-tetrazol-5-yl)- phenyl]piperazin- 1-yl]methyl]-1,3- benzothiazole | Z | 447.56 | 448.3 |
| A-55 | 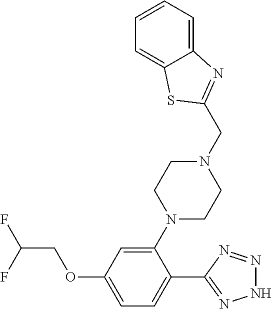 [SMILES: FC(F)COc1ccc(-c2nn[nH]n2)c(N2CCN(Cc3nc4ccccc4s3)CC2)c1] | 2-[[4-[5-(2,2- difluoroethoxy)-2- (2H-tetrazol-5-yl)- phenyl]piperazin- 1-yl]methyl]-1,3- benzothiazole | Z | 457.50 | 458.2 |
| A-56 | 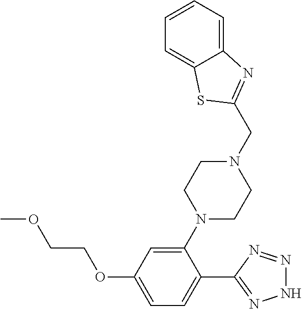 [SMILES: COCCOc1ccc(-c2nn[nH]n2)c(N2CCN(Cc3nc4ccccc4s3)CC2)c1] | 2-[[4-[5-(2-meth- oxyethoxy)-2-(2H- tetrazol-5-yl)phen- yl]piperazin-1-yl]- methyl]-1,3-benzo- thiazole | Z | 451.54 | 452.3 |
| A-57 | 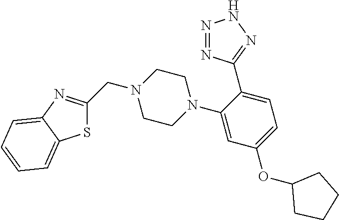 [SMILES: c1ccc2sc(CN3CCN(c4cc(OC5CCCC5)ccc4-c4nn[nH]n4)CC3)nc2c1] | 2-[[4-[5-(cyclo- pentoxy)-2-(2H- tetrazol-5-yl)- phenyl]piperazin- 1-yl]methyl]-1,3- benzothiazole | Z | 461.58 | 462.3 |
| A-58 | 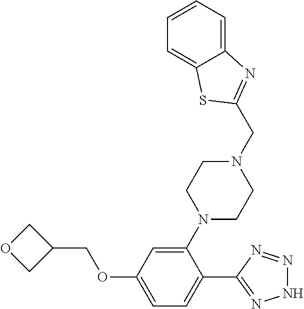 [SMILES: c1ccc2sc(CN3CCN(c4cc(OCC5COC5)ccc4-c4nn[nH]n4)CC3)nc2c1] | 2-[[4-[5-(oxetan-3- ylmethoxy)-2-(2H- tetrazol-5-yl)phen- yl]piperazin-1-yl]- methyl]-1,3-benzo- thiazole | Z | 463.56 | 464.3 |
| A-59 | 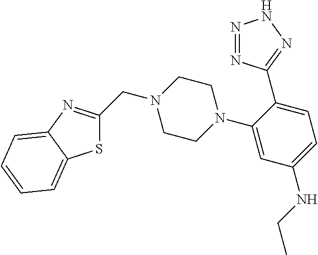 [SMILES: CCNc1ccc(-c2nn[nH]n2)c(N2CCN(Cc3nc4ccccc4s3)CC2)c1] | 3-[4-(1,3-benzothi- azol-2-ylmethyl)- piperazin-1-yl]- N-ethyl-4-(2H- tetrazol-5-yl)aniline | AA | 420.53 | 421.3 |
| A-60 | 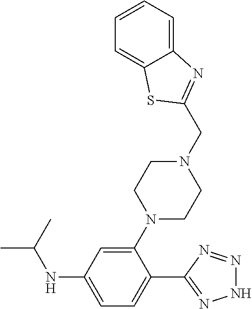 [SMILES: CC(C)Nc1ccc(-c2nn[nH]n2)c(N2CCN(Cc3nc4ccccc4s3)CC2)c1] | 3-[4-(1,3-benzothi- azol-2-ylmethyl)- piperazin-1-yl]- N-isopropyl-4-(2H- tetrazol-5-yl)aniline | AA | 434.56 | 435.3 |
| A-61 | 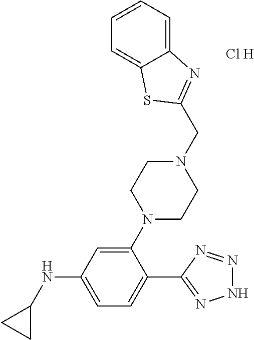 | 3-[4-(1,3-benzothi- azol-2-ylmethyl)- piperazin-1-yl]-N- cyclopropyl-4-(2H- tetrazol-5-yl)aniline hydrochloride | AA | 469.01 | 433.3 |
| A-62 | 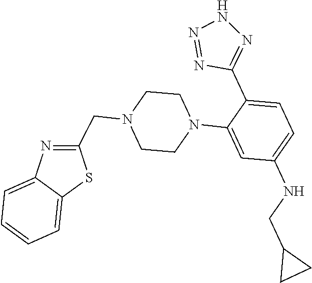 [SMILES: c1ccc2sc(CN3CCN(c4cc(NCC5CC5)ccc4-c4nn[nH]n4)CC3)nc2c1] | 3-[4-(1,3-benzothi- azol-2-ylmethyl)- piperazin-1-yl]-N- (cyclopropylmeth- yl)-4-(2H-tetrazol- 5-yl)aniline | AA | 446.57 | 461.3 |
| A-63 | 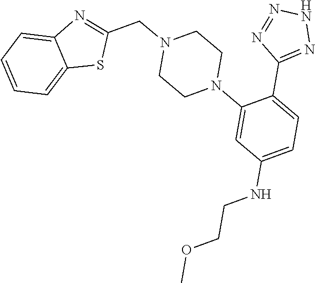 [SMILES: COCCNc1ccc(-c2nn[nH]n2)c(N2CCN(Cc3nc4ccccc4s3)CC2)c1] | 3-[4-(1,3-benzothi- azol-2-ylmethyl)- piperazin-1-yl]-N- (2-methoxyethyl)- 4-(2H-tetrazol-5- yl)aniline | AA | 450.56 | 451.2 |
| A-64 | 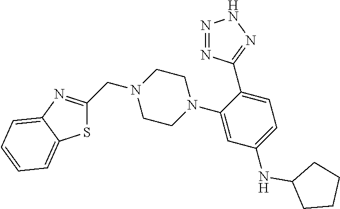 [SMILES: c1ccc2sc(CN3CCN(c4cc(NC5CCCC5)ccc4-c4nn[nH]n4)CC3)nc2c1] | 3-[4-(1,3-benzothi- azol-2-ylmethyl)- piperazin-1-yl]-N- cyclopentyl-4-(2H- tetrazol-5-yl)aniline | AA | 460.60 | 461.3 |
| A-65 | 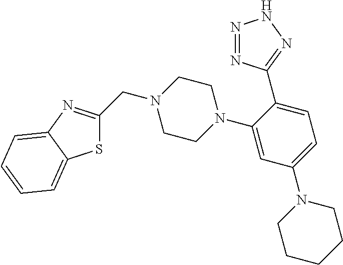 [SMILES: c1ccc2sc(CN3CCN(c4cc(N5CCCCC5)ccc4-c4nn[nH]n4)CC3)nc2c1] | 2-[[4-[5-(1-piperid- yl)-2-(2H-tetrazol-5- yl)phenyl]piperazin- 1-yl]methyl]-1,3- benzothiazole | AA | 460.60 | 461.3 |
| A-66 | 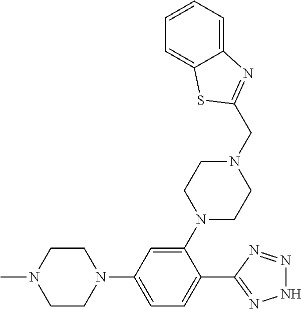 [SMILES: CN1CCN(c2ccc(-c3nn[nH]n3)c(N3CCN(Cc4nc5ccccc5s4)CC3)c2)CC1] | 2-[[4-[5-(4-methyl- piperazin-1-yl)-2- (2H-tetrazol-5-yl)- phenyl]piperazin- 1-yl]methyl]-1,3- benzothiazole | AA | 475.61 | 476.3 |
| A-67 | 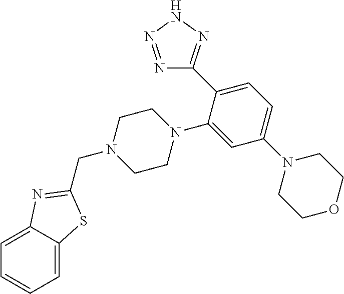 [SMILES: c1ccc2sc(CN3CCN(c4cc(N5CCOCC5)ccc4-c4nn[nH]n4)CC3)nc2c1] | 4-[3-[4-(1,3-benzo- thiazol-2-ylmethyl)- piperazin-1-yl]-4- (2H-tetrazol-5-yl)- phenyl]morpholine | AA | 462.57 | 463.3 |
| A-68 | 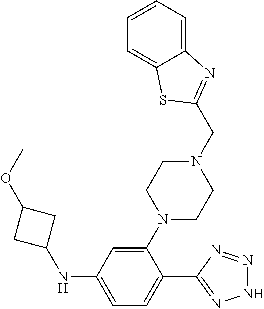 [SMILES: COC1CC(Nc2ccc(-c3nn[nH]n3)c(N3CCN(Cc4nc5ccccc5s4)CC3)c2)C1] | 3-[4-(1,3-benzo- thiazol-2-ylmethyl)- piperazin-1-yl]-N- (3-methoxycyclo- butyl)-4-(2H- tetrazol-5-yl)aniline | AA | 476.60 | 477.3 |
| A-69 | 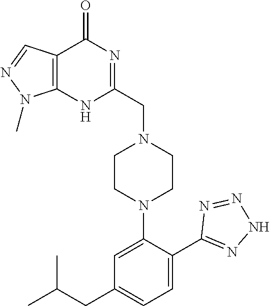 [SMILES: CC(C)Cc1ccc(-c2nn[nH]n2)c(N2CCN(Cc3nc(=O)c4cnn(C)c4[nH]3)CC2)c1] | 6-[[4-[5-isobutyl-2- (2H-tetrazol-5-yl)- phenyl]piperazin- 1-yl]methyl]-1- methyl-7H-pyr- azolo[3,4-d]pyrimi- din-4-one | U | 448.52 | 449.3 |
| A-70 | 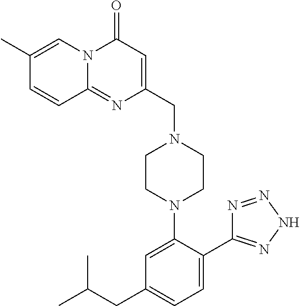 [SMILES: Cc1ccc2nc(CN3CCN(c4cc(CC(C)C)ccc4-c4nn[nH]n4)CC3)cc(=O)n2c1] | 2-[[4-[5-isobutyl-2- (2H-tetrazol-5-yl)- phenyl]piperazin- 1-yl]methyl]-7- methyl-pyrido[1,2- a]pyrimidin-4-one | U | 458.56 | 459.3 |
| A-71 | 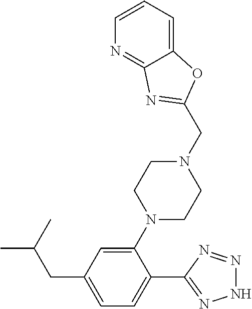 [SMILES: CC(C)Cc1ccc(-c2nn[nH]n2)c(N2CCN(Cc3nc4ncccc4o3)CC2)c1] | 2-[4-[5-isobutyl-2- (2H-tetrazol-5-yl)- phenyl]piperazin-1- yl]methyl]oxazolo- [4,5-b]pyridine | U | 418.49 | 437.3 |
| A-72 | 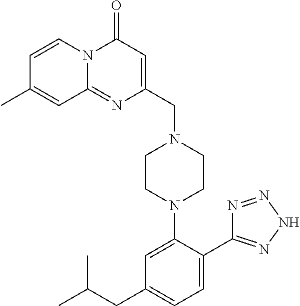 [SMILES: Cc1ccn2c(=O)cc(CN3CCN(c4cc(CC(C)C)ccc4-c4nn[nH]n4)CC3)nc2c1] | 2-[[4-[5-isobutyl-2- (2H-tetrazol-5-yl)- phenyl]piperazin- 1-yl]methyl]-8- methyl-pyrido[1,2- a]pyrimidin-4-one | U | 458.56 | 459.3 |
| A-73 | 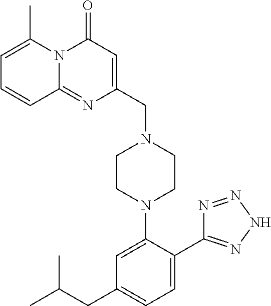 [SMILES: Cc1cccc2nc(CN3CCN(c4cc(CC(C)C)ccc4-c4nn[nH]n4)CC3)cc(=O)n12] | 2-[[4-[5-isobutyl-2- (2H-tetrazol-5-yl)- phenyl]piperazin- 1-yl]methyl]-6- methyl-pyrido[1,2- a]pyrimidin-4-one- | U | 458.56 | 459.3 |
| A-74 | 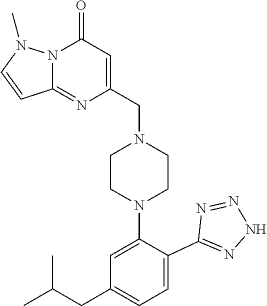 [SMILES: CC(C)Cc1ccc(-c2nn[nH]n2)c(N2CCN(Cc3cc(=O)n4c(ccn4C)n3)CC2)c1] | 5-[[4-[5-isobutyl-2- (2H-tetrazol-5-yl)- phenyl]piperazin- 1-yl]methyl]-1- methyl-pyrazolo- [1,5-a]pyrimidin-7- one | U | 447.54 | 448.3 |
| A-75 | 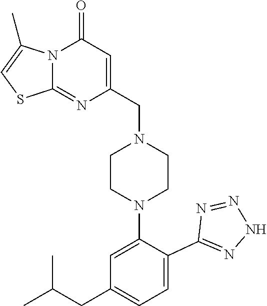 [SMILES: Cc1csc2nc(CN3CCN(c4cc(CC(C)C)ccc4-c4nn[nH]n4)CC3)cc(=O)n12] | 7-[4-[5-isobutyl-2- (2H-tetrazol-5-yl)- phenyl]piperazin- 1-yl]methyl]-3- methyl-thiazolo[3,2- a]pyrimidin-5-one | U | 464.59 | 465.3 |
| A-76 | 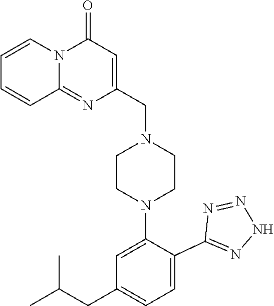 [SMILES: CC(C)Cc1ccc(-c2nn[nH]n2)c(N2CCN(Cc3cc(=O)n4ccccc4n3)CC2)c1] | 2-[[4-[5-isobutyl-2- (2H-tetrazol-5-yl)- phenyl]piperazin- 1-yl]methyl]pyrido- [1,2-a]pyrimidin-4- one | U | 444.53 | 445.3 |
| A-77 | 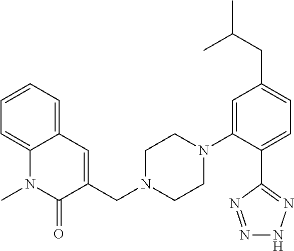 [SMILES: CC(C)Cc1ccc(-c2nn[nH]n2)c(N2CCN(Cc3cc4ccccc4n(C)c3=O)CC2)c1] | 3-[[4-[5-isobutyl-2- (2H-tetrazol-5-yl)- phenyl]piperazin- 1-yl]methyl]-1- methyl-quinolin-2- one | U | 457.57 | 458.4 |
| A-78 | 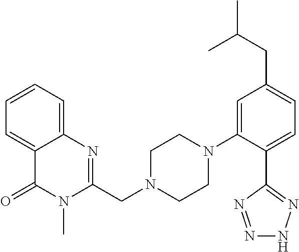 [SMILES: CC(C)Cc1ccc(-c2nn[nH]n2)c(N2CCN(Cc3nc4ccccc4c(=O)n3C)CC2)c1] | 2-[[4-[5-isobutyl-2- (2H-tetrazol-5-yl)- phenyl]piperazin- 1-yl]methyl]-3- methyl-quinazolin- 4-one | U | 458.56 | 459.3 |
| A-79 | 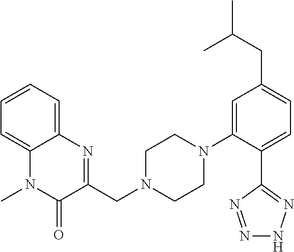 [SMILES: CC(C)Cc1ccc(-c2nn[nH]n2)c(N2CCN(Cc3nc4ccccc4n(C)c3=O)CC2)c1] | 3-[[4-[5-isobutyl-2- (2H-tetrazol-5-yl)- phenyl]piperazin- 1-yl]methyl]-1- methyl-quinoxalin- 2-one | U | 458.56 | 459.3 |
| A-80 | 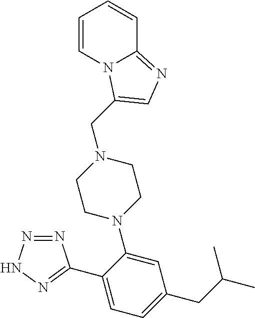 [SMILES: CC(C)Cc1ccc(-c2nn[nH]n2)c(N2CCN(Cc3cnc4ccccn34)CC2)c1] | 3-[[4-[5-isobutyl-2- (2H-tetrazol-5-yl)- phenyl]piperazin- 1-yl]methyl]imid- azo[1,2-a]pyridine | W | 416.52 | 417.3 |
| A-81 | 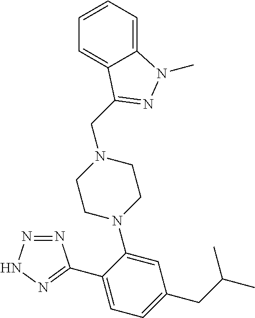 [SMILES: CC(C)Cc1ccc(-c2nn[nH]n2)c(N2CCN(Cc3nn(C)c4ccccc34)CC2)c1] | 3-[[4-[5-isobutyl-2- (2H-tetrazol-5-yl)- phenyl]piperazin- 1-yl]methyl]-1- methyl-indazole | W | 430.55 | 431.3 |
| A-82 | 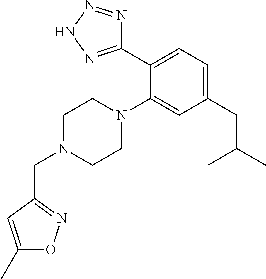 [SMILES: Cc1cc(CN2CCN(c3cc(CC(C)C)ccc3-c3nn[nH]n3)CC2)no1] | 3-[[4-[5-isobutyl-2- (2H-tetrazol-5-yl)- phenyl]piperazin- 1-yl]methyl]-5- methyl-isoxazole | U | 381.47 | 382.3 |
| A-83 | 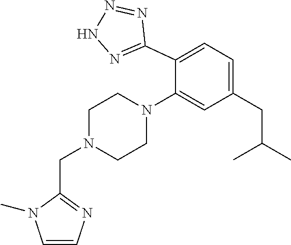 [SMILES: CC(C)Cc1ccc(-c2nn[nH]n2)c(N2CCN(Cc3nccn3C)CC2)c1] | 1-[5-isobutyl-2- (2H-tetrazol-5-yl)- phenyl]-4-[(1-meth- ylimidazol-2-yl)- methyl]piperazine | U | 380.49 | 381.3 |
| A-84 | 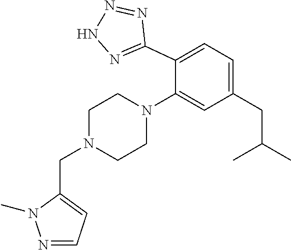 [SMILES: CC(C)Cc1ccc(-c2nn[nH]n2)c(N2CCN(Cc3ccnn3C)CC2)c1] | 1-[5-isobutyl-2-(2H- tetrazol-5-yl)phen- yl]-4-[(2-methylpyr- azol-3-yl)methyl]- piperazine | U | 380.49 | 381.3 |
| A-85 | 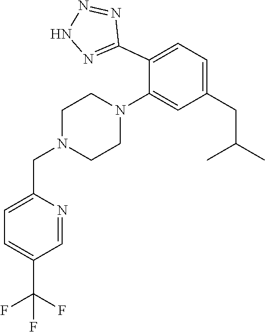 [SMILES: CC(C)Cc1ccc(-c2nn[nH]n2)c(N2CCN(Cc3ccc(C(F)(F)F)cn3)CC2)c1] | 1-[5-isobutyl-2- (2H-tetrazol-5-yl)- phenyl]-4-[[5- (trifluoromethyl)-2- pyridyl]methyl]- piperazine | U | 445.48 | 446.3 |
| A-86 | 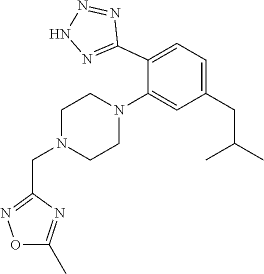 [SMILES: Cc1nc(CN2CCN(c3cc(CC(C)C)ccc3-c3nn[nH]n3)CC2)no1] | 3-[[4-[5-isobutyl-2- (2H-tetrazol-5-yl)- phenyl]piperazin-1- yl]methyl]-5-meth- yl-1,2,4-oxadiazole | U | 382.46 | 383.3 |
| A-87 | 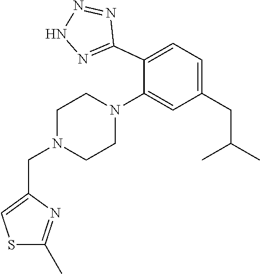 [SMILES: Cc1nc(CN2CCN(c3cc(CC(C)C)ccc3-c3nn[nH]n3)CC2)cs1] | 4-[4-[5-isobutyl-2- (2H-tetrazol-5-yl)- phenyl]piperazin- 1-yl]methyl]-2- methyl-thiazole | U | 397.54 | 398.3 |
| A-88 | 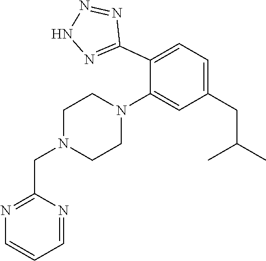 [SMILES: CC(C)Cc1ccc(-c2nn[nH]n2)c(N2CCN(Cc3ncccn3)CC2)c1] | 2-[4-[5-isobutyl-2- (2H-tetrazol-5-yl)- phenyl]piperazin- 1-yl]methyl]pyrimi- dine | U | 378.47 | 379.3 |
| A-89 | 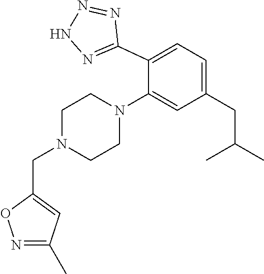 [SMILES: Cc1cc(CN2CCN(c3cc(CC(C)C)ccc3-c3nn[nH]n3)CC2)on1] | 5-[[4-[5-isobutyl-2- (2H-tetrazol-5-yl)- phenyl]piperazin- 1-yl]methyl]-3- methyl-isoxazole | U | 381.47 | 382.3 |
| A-90 | 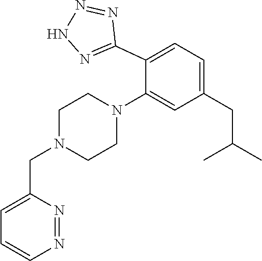 [SMILES: CC(C)Cc1ccc(-c2nn[nH]n2)c(N2CCN(Cc3cccnn3)CC2)c1] | 3-[[4-[5-isobutyl-2- (2H-tetrazol-5-yl)- phenyl]piperazin- 1-yl]methyl]pyrid- azine | U | 378.47 | 379.3 |
| A-91 | 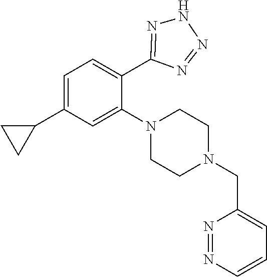 [SMILES: c1cnnc(CN2CCN(c3cc(C4CC4)ccc3-c3nn[nH]n3)CC2)c1] | 3-[[4-[5-cycloprop- yl-2-(2H-tetrazol-5- yl)phenyl]piperazin- 1-yl]methyl]pyrid- azine | D | 362.43 | 363.3 |
| A-92 | 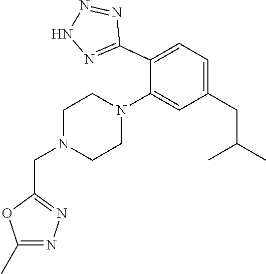 [SMILES: Cc1nnc(CN2CCN(c3cc(CC(C)C)ccc3-c3nn[nH]n3)CC2)o1] | 2-[4-[5-isobutyl-2- (2H-tetrazol-5-yl)- phenyl]piperazin- 1-yl]methyl]-5- methyl-1,3,4-oxa- diazole | U | 382.46 | 383.3 |
| A-93 | 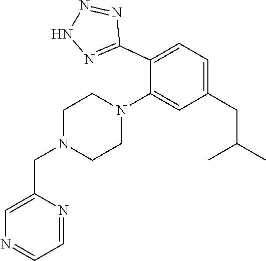 [SMILES: CC(C)Cc1ccc(-c2nn[nH]n2)c(N2CCN(Cc3cnccn3)CC2)c1] | 2-[[4-[5-isobutyl-2- (2H-tetrazol-5-yl)- phenyl]piperazin- 1-yl]methyl]pyr- azine | U | 378.47 | 379.3 |
| A-94 | 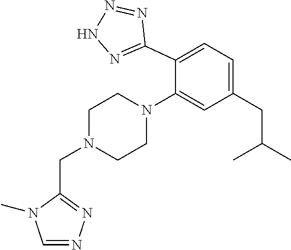 [SMILES: CC(C)Cc1ccc(-c2nn[nH]n2)c(N2CCN(Cc3nncn3C)CC2)c1] | 1-[5-isobutyl-2-(2H- tetrazol-5-yl)phen- yl]-4-[(4-methyl- 1,2,4-triazol-3-yl)- methyl]piperazine | U | 381.48 | 382.3 |
| A-95 | 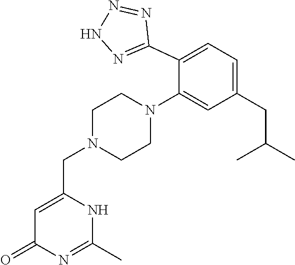 [SMILES: Cc1nc(=O)cc(CN2CCN(c3cc(CC(C)C)ccc3-c3nn[nH]n3)CC2)[nH]1] | 6-[[4-[5-isobutyl-2- (2H-tetrazol-5-yl)- phenyl]piperazin- 1-yl]methyl]-2- methyl-1H-pyrimi- din-4-one | U | 408.50 | 409.3 |
| A-96 | 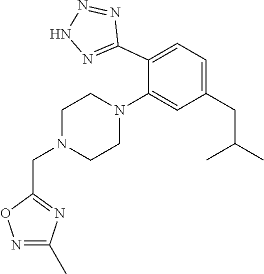 [SMILES: Cc1noc(CN2CCN(c3cc(CC(C)C)ccc3-c3nn[nH]n3)CC2)n1] | 5-[[4-[5-isobutyl-2- (2H-tetrazol-5-yl)- phenyl]piperazin- 1-yl]methyl]-3- methyl-1,2,4-oxa- diazole | U | 382.46 | 383.3 |
| A-97 | 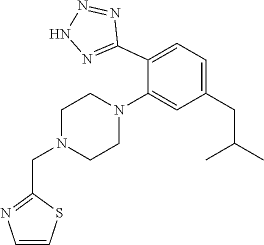 [SMILES: CC(C)Cc1ccc(-c2nn[nH]n2)c(N2CCN(Cc3nccs3)CC2)c1] | 2-[[4-[5-isobutyl-2- (2H-tetrazol-5-yl)- phenyl]piperazin-1- yl]methyl]thiazole | U | 383.51 | 384.3 |
| A-98 | 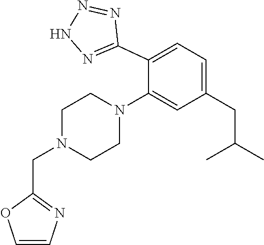 [SMILES: CC(C)Cc1ccc(-c2nn[nH]n2)c(N2CCN(Cc3ncco3)CC2)c1] | 2-[[4-[5-isobutyl-2- (2H-tetrazol-5-yl)- phenyl]piperazin-1- yl]methyl]oxazole | U | 367.45 | 368.3 |
| A-99 | 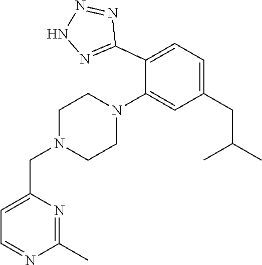 [SMILES: Cc1nccc(CN2CCN(c3cc(CC(C)C)ccc3-c3nn[nH]n3)CC2)n1] | 4-[4-[5-isobutyl-2- (2H-tetrazol-5-yl)- phenyl]piperazin- 1-yl]methyl]-2- methyl-pyrimidine | U | 392.50 | 393.3 |
| A-100 | 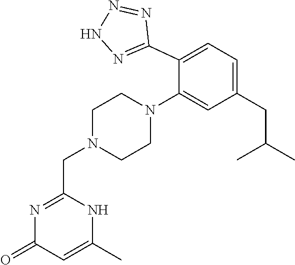 [SMILES: Cc1cc(=O)nc(CN2CCN(c3cc(CC(C)C)ccc3-c3nn[nH]n3)CC2)[nH]1] | 2-[[4-[5-isobutyl-2- (2H-tetrazol-5-yl)- phenyl]piperazin- 1-yl]methyl]-6- methyl-1H-pyrimi- din-4-one | U | 408.50 | 409.3 |
| A-101 | 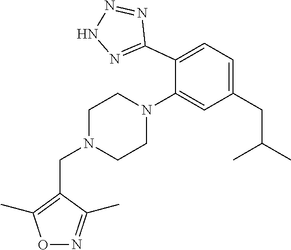 [SMILES: Cc1noc(C)c1CN1CCN(c2cc(CC(C)C)ccc2-c2nn[nH]n2)CC1] | 4-[4-[5-isobutyl-2- (2H-tetrazol-5-yl)- phenyl]piperazin- 1-yl]methyl]-3,5- dimethyl-isoxazole | U | 395.50 | 396.3 |
| A-102 | 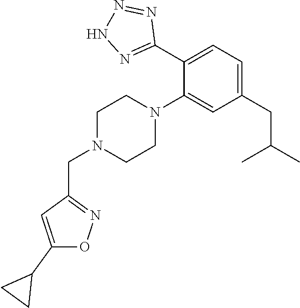 [SMILES: CC(C)Cc1ccc(-c2nn[nH]n2)c(N2CCN(Cc3cc(C4CC4)on3)CC2)c1] | 5-cyclopropyl-3-[4- [5-isobutyl-2-(2H- tetrazol-5-yl)phen- yl]piperazin-1-yl]- methyl]isoxazole | U | 407.51 | 408.3 |
| A-103 | 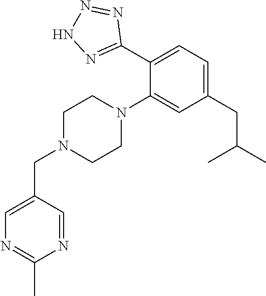 [SMILES: Cc1ncc(CN2CCN(c3cc(CC(C)C)ccc3-c3nn[nH]n3)CC2)cn1] | 5-[[4-[5-isobutyl-2- (2H-tetrazol-5-yl)- phenyl]piperazin- 1-yl]methyl]-2- methyl-pyrimidine | U | 392.50 | 393.4 |
| A-104 | 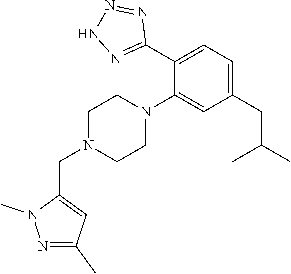 [SMILES: Cc1cc(CN2CCN(c3cc(CC(C)C)ccc3-c3nn[nH]n3)CC2)n(C)n1] | 1-[(2,5-dimethyl- pyrazol-3-yl)meth- yl]-4-[5-isobutyl-2- (2H-tetrazol-5-yl)- phenyl]piperazine | U | 394.52 | 395.3 |
| A-105 | 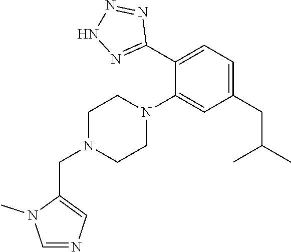 [SMILES: CC(C)Cc1ccc(-c2nn[nH]n2)c(N2CCN(Cc3cncn3C)CC2)c1] | 1-[5-isobutyl-2-(2H- tetrazol-5-yl)phen- yl]-4-[(3-methyl- imidazol-4-yl)meth- yl]piperazine | U | 380.49 | 381.3 |
| A-106 | 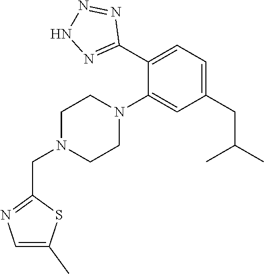 [SMILES: Cc1cnc(CN2CCN(c3cc(CC(C)C)ccc3-c3nn[nH]n3)CC2)s1] | 2-[[4-[5-isobutyl-2- (2H-tetrazol-5-yl)- phenyl]piperazin- 1-yl]methyl]-5- methyl-thiazole | U | 397.54 | 398.3 |
| A-107 | 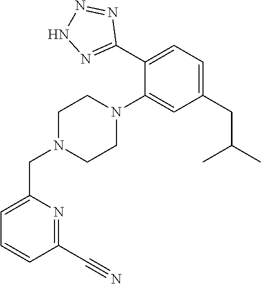 [SMILES: CC(C)Cc1ccc(-c2nn[nH]n2)c(N2CCN(Cc3cccc(C#N)n3)CC2)c1] | 6-[[4-[5-isobutyl-2- (2H-tetrazol-5-yl)- phenyl]piperazin- 1-yl]methyl]pyr- idine-2-carbonitrile | V | 402.50 | 403.3 |
| A-108 | 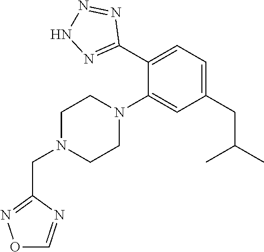 [SMILES: CC(C)Cc1ccc(-c2nn[nH]n2)c(N2CCN(Cc3ncon3)CC2)c1] | 3-[[4-[5-isobutyl-2- (2H-tetrazol-5-yl)- phenyl]piperazin- 1-yl]methyl]-1,2,4- oxadiazole | V | 368.44 | 369.3 |
| A-109 | 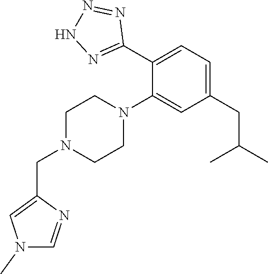 [SMILES: CC(C)Cc1ccc(-c2nn[nH]n2)c(N2CCN(Cc3cn(C)cn3)CC2)c1] | 1-[5-isobutyl-2-(2H- tetrazol-5-yl)phen- yl]-4-[(1-methyl- imidazol-4-yl)meth- yl]piperazine | U | 380.49 | 381.3 |
| A-110 | 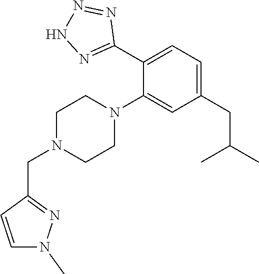 [SMILES: CC(C)Cc1ccc(-c2nn[nH]n2)c(N2CCN(Cc3ccn(C)n3)CC2)c1] | 1-[5-isobutyl-2-(2H- tetrazol-5-yl)phen- yl]-4-[(1-methyl- pyrazol-3-yl)meth- yl]piperazine | U | 380.49 | 381.3 |
| A-111 | 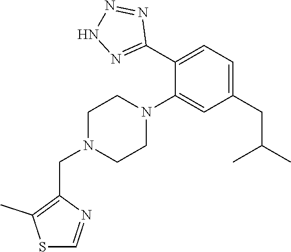 [SMILES: Cc1scnc1CN1CCN(c2cc(CC(C)C)ccc2-c2nn[nH]n2)CC1] | 4-[[4-[5-isobutyl-2- (2H-tetrazol-5-yl)- phenyl]piperazin- 1-yl]methyl]-5- methyl-thiazole | U | 397.54 | 398.3 |
| A-112 | 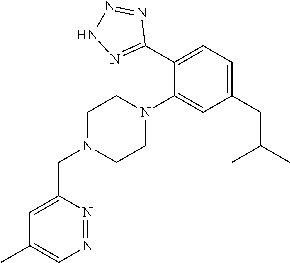 [SMILES: Cc1cnnc(CN2CCN(c3cc(CC(C)C)ccc3-c3nn[nH]n3)CC2)c1] | 3-[4-[5-isobutyl-2- (2H-tetrazol-5-yl)- phenyl]piperazin- 1-yl]methyl]-5- methyl-pyridazine | U | 392.50 | 393.4 |
| A-113 | 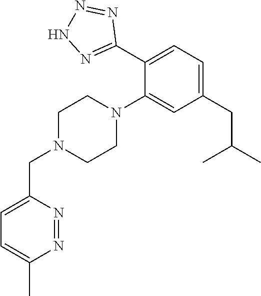 [SMILES: Cc1ccc(CN2CCN(c3cc(CC(C)C)ccc3-c3nn[nH]n3)CC2)nn1] | 3-[4-[5-isobutyl-2- (2H-tetrazol-5-yl)- phenyl]piperazin- 1-yl]methyl]-6- methyl-pyridazine | U | 392.50 | 393.4 |
| A-114 | 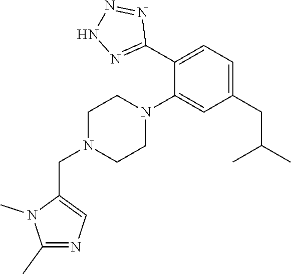 [SMILES: Cc1ncc(CN2CCN(c3cc(CC(C)C)ccc3-c3nn[nH]n3)CC2)n1C] | 1-[(2,3-dimethyl- imidazol-4-yl)meth- yl]-4-[5-isobutyl-2- (2H-tetrazol-5-yl)- phenyl]piperazine | W | 394.52 | 395.4 |
| A-115 | 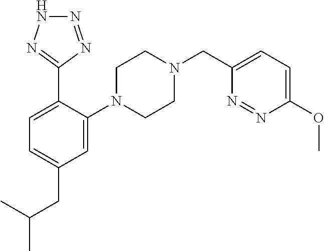 [SMILES: COc1ccc(CN2CCN(c3cc(CC(C)C)ccc3-c3nn[nH]n3)CC2)nn1] | 3-[[4-[5-isobutyl-2- (2H-tetrazol-5-yl)- phenyl]piperazin- 1-yl]methyl]-6- methoxy-pyridazine | U | 408.50 | 409.3 |
| A-116 | 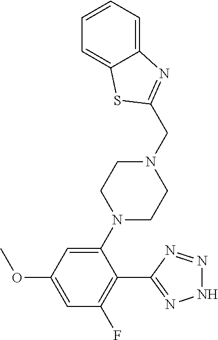 [SMILES: COc1cc(F)c(-c2nn[nH]n2)c(N2CCN(Cc3nc4ccccc4s3)CC2)c1] | 2-[[4-[3-fluoro-5- methoxy-2-(2H- tetrazol-5-yl)phen- yl]piperazin-1-yl]- methyl]-1,3-benzo- thiazole | C | 425.48 | 426.3 |
| A-117 | 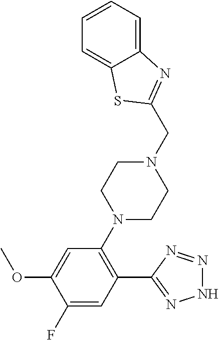 [SMILES: COc1cc(N2CCN(Cc3nc4ccccc4s3)CC2)c(-c2nn[nH]n2)cc1F] | 2-[[4-[4-fluoro-5- methoxy-2-(2H- tetrazol-5-yl)phen- yl]piperazin-1-yl]- methyl]-1,3-benzo- thiazole | C | 425.48 | 426.2 |
| A-118 | 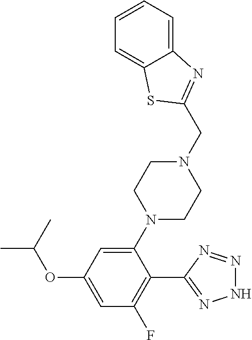 [SMILES: CC(C)Oc1cc(F)c(-c2nn[nH]n2)c(N2CCN(Cc3nc4ccccc4s3)CC2)c1] | 2-[[4-[3-fluoro-5- isopropoxy-2-(2H- tetrazol-5-yl)phen- yl]piperazin-1-yl]- methyl]-1,3-benzo- thiazole | C | 453.54 | 454.2 |
| A-119 | 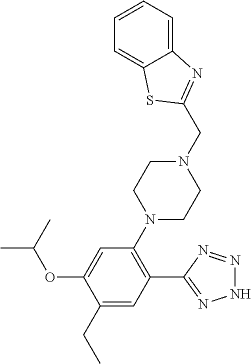 [SMILES: CCc1cc(-c2nn[nH]n2)c(N2CCN(Cc3nc4ccccc4s3)CC2)cc1OC(C)C] | 2-[[4-[4-ethyl-5- isopropoxy-2-(2H- tetrazol-5-yl)phen- yl]piperazin-1-yl]- methyl]-1,3-benzo- thiazole | C | 463.60 | 464.3 |
| A-120 | 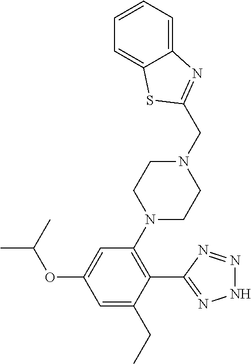 [SMILES: CCc1cc(OC(C)C)cc(N2CCN(Cc3nc4ccccc4s3)CC2)c1-c1nn[nH]n1] | 2-[[4-[3-ethyl-5-iso- propoxy-2-(2H- tetrazol-5-yl)phen- yl]piperazin-1-yl]- methyl]-1,3-benzo- thiazole | C | 463.60 | 464.3 |
| A-121 | 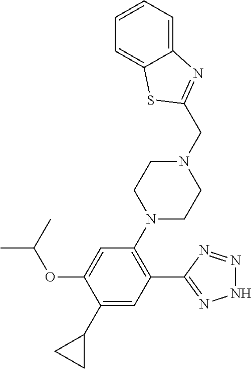 [SMILES: CC(C)Oc1cc(N2CCN(Cc3nc4ccccc4s3)CC2)c(-c2nn[nH]n2)cc1C1CC1] | 2-[[4-[4-cycloprop- yl-5-isopropoxy-2- (2H-tetrazol-5-yl)- phenyl]piperazin- 1-yl]methyl]-1,3- benzothiazole | C | 475.61 | 476.3 |
| A-122 | 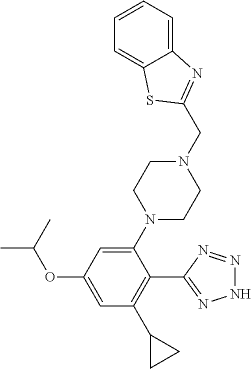 [SMILES: CC(C)Oc1cc(C2CC2)c(-c2nn[nH]n2)c(N2CCN(Cc3nc4ccccc4s3)CC2)c1] | 2-[[4-[3-cycloprop- yl-5-isopropoxy-2- (2H-tetrazol-5-yl)- phenyl]piperazin- 1-yl]methyl]-1,3- benzothiazole | C | 475.61 | 476.3 |
| A-123 | 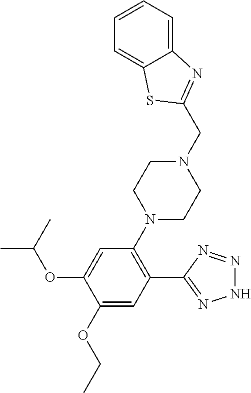 [SMILES: CCOc1cc(-c2nn[nH]n2)c(N2CCN(Cc3nc4ccccc4s3)CC2)cc1OC(C)C] | 2-[[4-[4-ethoxy-5- isopropoxy-2-(2H- tetrazol-5-yl)phen- yl]piperazin-1-yl]- methyl]-1,3-benzo- thiazole | C | 479.60 | 480.3 |
| A-124 | 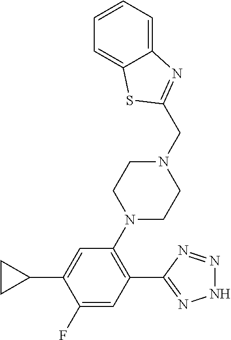 [SMILES: Fc1cc(-c2nn[nH]n2)c(N2CCN(Cc3nc4ccccc4s3)CC2)cc1C1CC1] | 2-[[4-[5-cycloprop- yl-4-fluoro-2-(2H- tetrazol-5-yl)phen- yl]piperazin-1-yl]- methyl]-1,3-benzo- thiazole | C | 435.52 | 436.2 |
| A-125 | 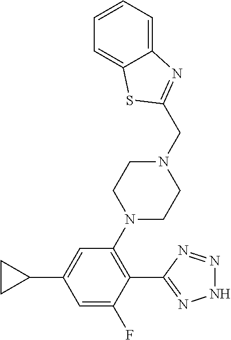 [SMILES: Fc1cc(C2CC2)cc(N2CCN(Cc3nc4ccccc4s3)CC2)c1-c1nn[nH]n1] | 2-[[4-[5-cycloprop- yl-3-fluoro-2-(2H- tetrazol-5-yl)phen- yl]piperazin-1-yl]- methyl]-1,3-benzo- thiazole | C | 435.52 | 436.2 |
| A-126 | 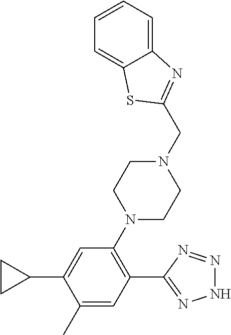 [SMILES: Cc1cc(-c2nn[nH]n2)c(N2CCN(Cc3nc4ccccc4s3)CC2)cc1C1CC1] | 2-[[4-[5-cycloprop- yl-4-methyl-2-(2H- tetrazol-5-yl)phen- yl]piperazin-1-yl]- methyl]-1,3-benzo- thiazole | C | 431.56 | 432.3 |
| A-127 | 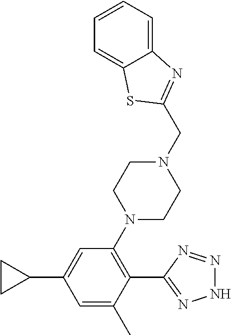 [SMILES: Cc1cc(C2CC2)cc(N2CCN(Cc3nc4ccccc4s3)CC2)c1-c1nn[nH]n1] | 2-[[4-[5-cycloprop- yl-3-methyl-2-(2H- tetrazol-5-yl)phen- yl]piperazin-1-yl]- methyl]-1,3-benzo- thiazole | C | 431.56 | 432.3 |
| A-128 | 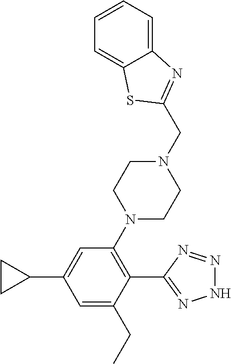 [SMILES: CCc1cc(C2CC2)cc(N2CCN(Cc3nc4ccccc4s3)CC2)c1-c1nn[nH]n1] | 2-[[4-[5-cycloprop- yl-3-ethyl-2-(2H- tetrazol-5-yl)phen- yl]piperazin-1-yl]- methyl]-1,3-benzo- thiazole | C | 445.58 | 446.2 |
| A-129 | 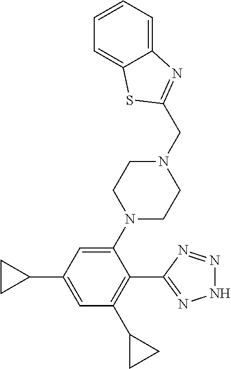 [SMILES: c1ccc2sc(CN3CCN(c4cc(C5CC5)cc(C5CC5)c4-c4nn[nH]n4)CC3)nc2c1] | 2-[[4-[3,5-dicyclo- propyl-2-(2H- tetrazol-5-yl)phen- yl]piperazin-1-yl]- methyl]-1,3-benzo- thiazole | C | 457.59 | 458.3 |
| A-130 | 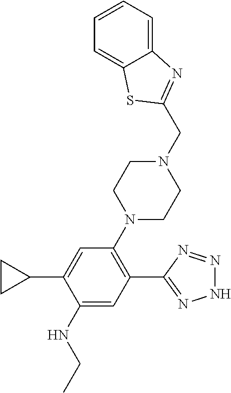 [SMILES: CCNc1cc(-c2nn[nH]n2)c(N2CCN(Cc3nc4ccccc4s3)CC2)cc1C1CC1] | 4-[4-(1,3-benzothi- azol-2-ylmethyl)- piperazin-1-yl]-2- cyclopropyl-N-eth- yl-5-(2H-tetrazol-5- yl)aniline | C | 460.60 | 461.2 |
| A-131 | 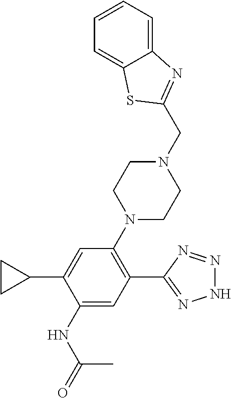 [SMILES: CC(=O)Nc1cc(-c2nn[nH]n2)c(N2CCN(Cc3nc4ccccc4s3)CC2)cc1C1CC1] | N-[4-[4-(1,3-benzo- thiazol-2-ylmethyl)- piperazin-1-yl]-2- cyclopropyl-5-(2H- tetrazol-5-yl)phen- yl]acetamide | C | 474.58 | 475.3 |
| A-132 | 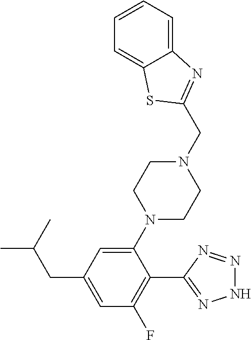 [SMILES: CC(C)Cc1cc(F)c(-c2nn[nH]n2)c(N2CCN(Cc3nc4ccccc4s3)CC2)c1] | 2-[[4-[3-fluoro-5- isobutyl-2-(2H- tetrazol-5-yl)phen- yl]piperazin-1-yl]- methyl]-1,3-benzo- thiazole | C | 451.56 | 452.3 |
| A-133 | 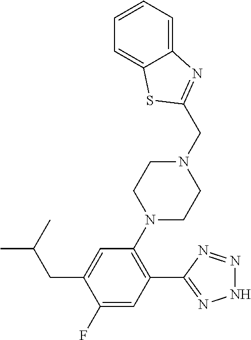 [SMILES: CC(C)Cc1cc(N2CCN(Cc3nc4ccccc4s3)CC2)c(-c2nn[nH]n2)cc1F] | 2-[[4-[4-fluoro-5- isobutyl-2-(2H- tetrazol-5-yl)phen- yl]piperazin-1-yl]- methyl]-1,3-benzo- thiazole | C | 451.56 | 452.2 |
| A-134 | 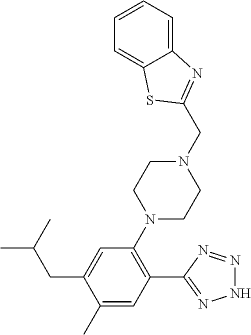 [SMILES: Cc1cc(-c2nn[nH]n2)c(N2CCN(Cc3nc4ccccc4s3)CC2)cc1CC(C)C] | 2-[[4-[5-isobutyl-4- methyl-2-(2H- tetrazol-5-yl)phen- yl]piperazin-1-yl]- methyl]-1,3-benzo- thiazole | C | 447.60 | 448.3 |
| A-135 | 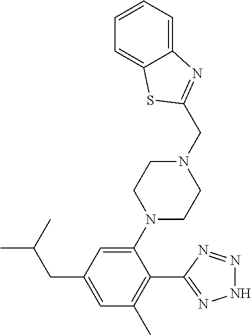 [SMILES: Cc1cc(CC(C)C)cc(N2CCN(Cc3nc4ccccc4s3)CC2)c1-c1nn[nH]n1] | 2-[4-[5-isobutyl-3- methyl-2-(2H- tetrazol-5-yl)phen- yl]piperazin-1-yl]- methyl]-1,3-benzo- thiazole | C | 447.60 | 448.3 |
| A-136 | 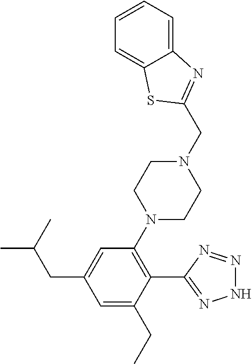 [SMILES: CCc1cc(CC(C)C)cc(N2CCN(Cc3nc4ccccc4s3)CC2)c1-c1nn[nH]n1] | 2-[[4-[3-ethyl-5- isobutyl-2-(2H- tetrazol-5-yl)phen- yl]piperazin-1-yl]- methyl]-1,3-benzo- thiazole | C | 461.63 | 462.3 |
| A-137 | 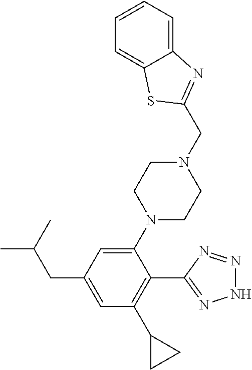 [SMILES: CC(C)Cc1cc(C2CC2)c(-c2nn[nH]n2)c(N2CCN(Cc3nc4ccccc4s3)CC2)c1] | 2-[[4-[3-cycloprop- yl-5-isobutyl-2- (2H-tetrazol-5-yl)- phenyl]piperazin- 1-yl]methyl]-1,3- benzothiazole | C | 473.64 | 474.3 |
| A-138 | 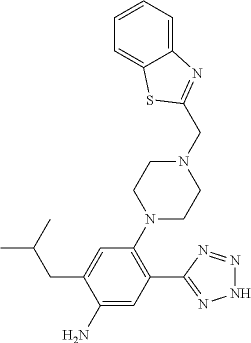 [SMILES: CC(C)Cc1cc(N2CCN(Cc3nc4ccccc4s3)CC2)c(-c2nn[nH]n2)cc1N] | 4-[4-(1,3-benzo- thiazol-2-ylmethyl)- piperazin-1-yl]-2- isobutyl-5-(2H- tetrazol-5-yl)aniline | C | 448.59 | 449.3 |
| A-139 | 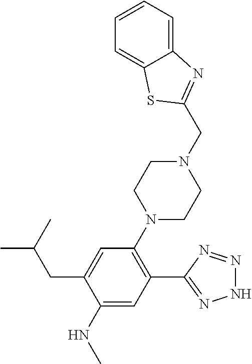 [SMILES: CNc1cc(-c2nn[nH]n2)c(N2CCN(Cc3nc4ccccc4s3)CC2)cc1CC(C)C] | 4-[4-(1,3-benzothi- azol-2-ylmethyl)- piperazin-1-yl]-2- isobutyl-N-methyl- 5-(2H-tetrazol-5- yl)aniline | C | 462.61 | 463.1 |
| A-140 | 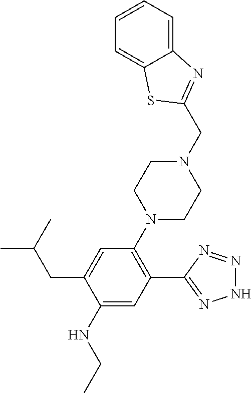 [SMILES: CCNc1cc(-c2nn[nH]n2)c(N2CCN(Cc3nc4ccccc4s3)CC2)cc1CC(C)C] | 4-[4-(1,3-benzo- thiazol-2-ylmethyl)- piperazin-1-yl]-N- ethyl-2-isobutyl-5- (2H-tetrazol-5-yl)- aniline | C | 476.64 | 477.2 |
| A-141 | 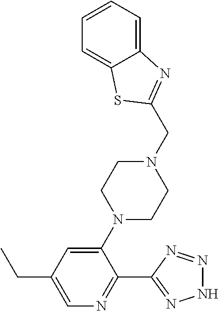 [SMILES: CCc1cnc(-c2nn[nH]n2)c(N2CCN(Cc3nc4ccccc4s3)CC2)c1] | 2-[4-[5-ethyl-2- (2H-tetrazol-5-yl)- 3-pyridyl]piperazin- 1-yl]methyl]-1,3- benzothiazole | C | 406.51 | 407.3 |
| A-142 | 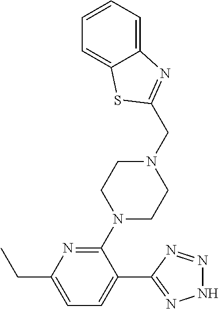 [SMILES: CCc1ccc(-c2nn[nH]n2)c(N2CCN(Cc3nc4ccccc4s3)CC2)n1] | 2-[[4-[6-ethyl-3- (2H-tetrazol-5-yl)- 2-pyridyl]piperazin- 1-yl]methyl]-1,3- benzothiazole | C | 406.51 | 407.2 |
| A-143 | 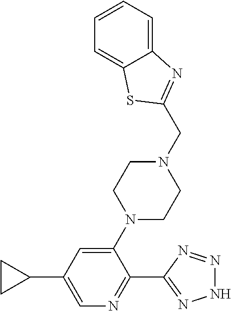 [SMILES: c1ccc2sc(CN3CCN(c4cc(C5CC5)cnc4-c4nn[nH]n4)CC3)nc2c1] | 2-[[4-[5-cycloprop- yl-2-(2H-tetrazol-5- yl)-3-pyridyl]piper- azin-1-yl]methyl]- 1,3-benzothiazole | C | 418.52 | 419.2 |
| A-144 | 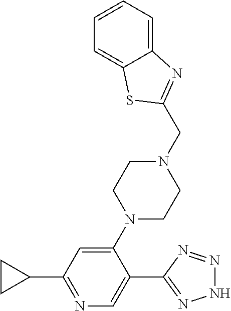 [SMILES: c1ccc2sc(CN3CCN(c4cc(C5CC5)ncc4-c4nn[nH]n4)CC3)nc2c1] | 2-[[4-[2-cycloprop- yl-5-(2H-tetrazol-5- yl)-4-pyridyl]piper- azin-1-yl]methyl]- 1,3-benzothiazole | C | 418.52 | 419.3 |
| A-145 | 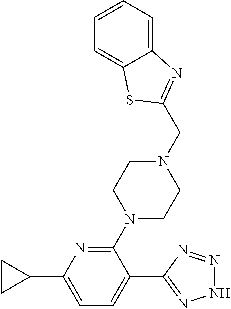 [SMILES: c1ccc2sc(CN3CCN(c4nc(C5CC5)ccc4-c4nn[nH]n4)CC3)nc2c1] | 2-[[4-[6-cycloprop- yl-3-(2H-tetrazol-5- yl)-2-pyridyl]piper- azin-1-yl]methyl]- 1,3-benzothiazole | C | 418.52 | 419.3 |
| A-146 | 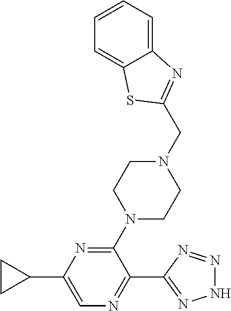 [SMILES: c1ccc2sc(CN3CCN(c4nc(C5CC5)cnc4-c4nn[nH]n4)CC3)nc2c1] | 2-[[4-[6-cycloprop- yl-3-(2H-tetrazol-5- yl)pyrazin-2-yl]- piperazin-1-yl]- methyl]-1,3-benzo- thiazole | C | 419.51 | 420.3 |
| A-147 | 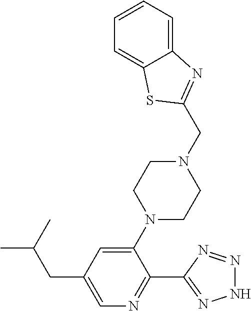 [SMILES: CC(C)Cc1cnc(-c2nn[nH]n2)c(N2CCN(Cc3nc4ccccc4s3)CC2)c1] | 2-[[4-[5-isobutyl-2- (2H-tetrazol-5-yl)- 3-pyridyl]piperazin- 1-yl]methyl]-1,3- benzothiazole | C | 434.56 | 435.3 |
| A-148 | 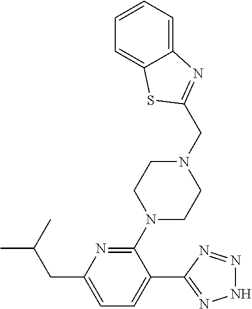 [SMILES: CC(C)Cc1ccc(-c2nn[nH]n2)c(N2CCN(Cc3nc4ccccc4s3)CC2)n1] | 2-[[4-[6-isobutyl-3- (2H-tetrazol-5-yl)- 2-pyridyl]piperazin- 1-yl]methyl]-1,3- benzothiazole | C | 434.56 | 435.2 |
| A-149 | 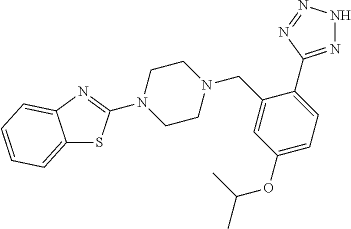 [SMILES: CC(C)Oc1ccc(-c2nn[nH]n2)c(CN2CCN(c3nc4ccccc4s3)CC2)c1] | 5-[4-(1,3-benzo- thiazol-2-ylmethyl)- piperazin-1-yl]-N- (cyclopropylmeth- yl)-6-(2H-tetrazol-5- yl)pyridin-3-amine | C | 447.56 | 448.3 |
| A-150 | 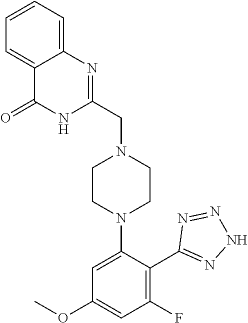 [SMILES: COc1cc(F)c(-c2nn[nH]n2)c(N2CCN(Cc3nc4ccccc4c(=O)[nH]3)CC2)c1] | 2-[[4-[3-fluoro-5- methoxy-2-(2H- tetrazol-5-yl)phen- yl]piperazin-1-yl]- methyl]-3H-quin- azolin-4-one | M | 436.44 | 437.3 |
| A-151 | 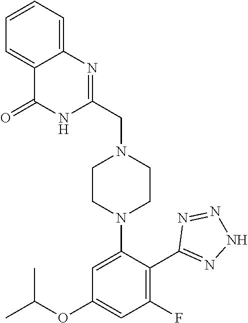 [SMILES: CC(C)Oc1cc(F)c(-c2nn[nH]n2)c(N2CCN(Cc3nc4ccccc4c(=O)[nH]3)CC2)c1] | 2-[[4-[3-fluoro-5- isopropoxy-2-(2H- tetrazol-5-yl)phen- yl]piperazin-1-yl]- methyl]-3H-quin- azolin-4-one | M | 464.50 | 465.3 |
| A-152 | 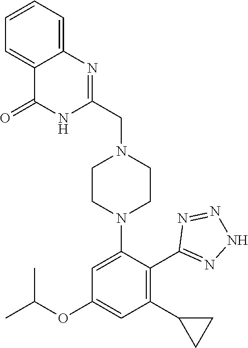 [SMILES: CC(C)Oc1cc(C2CC2)c(-c2nn[nH]n2)c(N2CCN(Cc3nc4ccccc4c(=O)[nH]3)CC2)c1] | 2-[4-[3-cyclopropyl- 5-isopropoxy-2- (2H-tetrazol-5-yl)- phenyl]piperazin- 1-yl]methyl]-3H- quinazolin-4-one | M | 486.57 | 487.3 |
| A-153 | 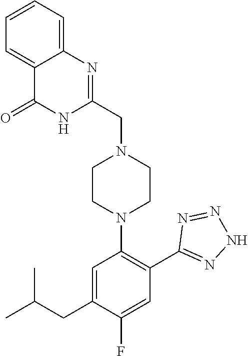 [SMILES: CC(C)Cc1cc(N2CCN(Cc3nc4ccccc4c(=O)[nH]3)CC2)c(-c2nn[nH]n2)cc1F] | 2-[[4-[4-fluoro-5- isobutyl-2-(2H- tetrazol-5-yl)phen- yl]piperazin-1-yl]- methyl]-3H-quin- azolin-4-one | M | 462.52 | 463.3 |
| A-154 | 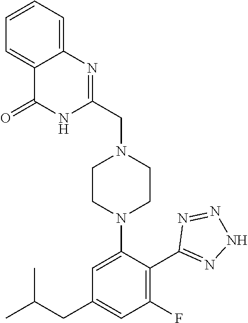 [SMILES: CC(C)Cc1cc(F)c(-c2nn[nH]n2)c(N2CCN(Cc3nc4ccccc4c(=O)[nH]3)CC2)c1] | 2-[[4-[3-fluoro-5- isobutyl-2-(2H- tetrazol-5-yl)phen- yl]piperazin-1-yl]- methyl]-3H-quin- azolin-4-one | M | 462.52 | 463.3 |
| A-155 | 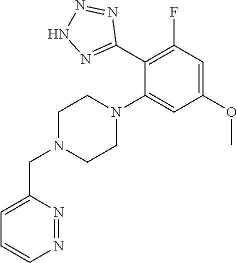 [SMILES: COc1cc(F)c(-c2nn[nH]n2)c(N2CCN(Cc3cccnn3)CC2)c1] | 3-[[4-[3-fluoro-5- methoxy-2-(2H- tetrazol-5-yl)phen- yl]piperazin-1-yl]- methyl]pyridazine | D | 370.38 | 371.3 |
| A-156 | 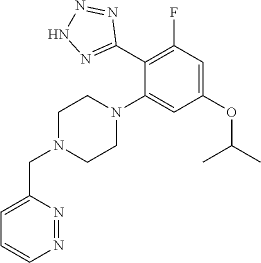 [SMILES: CC(C)Oc1cc(F)c(-c2nn[nH]n2)c(N2CCN(Cc3cccnn3)CC2)c1] | 3-[[4-[3-fluoro-5- isopropoxy-2-(2H- tetrazol-5-yl)phen- yl]piperazin-1-yl]- methyl]pyridazine | D | 398.44 | 399.3 |
| A-157 | 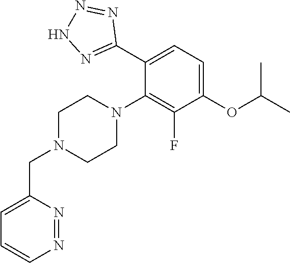 [SMILES: CC(C)Oc1ccc(-c2nn[nH]n2)c(N2CCN(Cc3cccnn3)CC2)c1F] | 3-[[4-[2-fluoro-3- isopropoxy-6-(2H- tetrazol-5-yl)phen- yl]piperazin-1-yl]- methyl]pyridazine | D | 398.44 | 399.3 |
| A-158 | 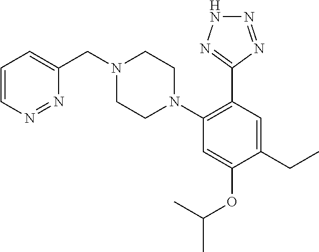 [SMILES: CCc1cc(-c2nn[nH]n2)c(N2CCN(Cc3cccnn3)CC2)cc1OC(C)C] | 3-[[4-[4-ethyl-5- isopropoxy-2-(2H- tetrazol-5-yl)phen- yl]piperazin-1-yl]- methyl]pyridazine | D | 408.50 | 409.3 |
| A-159 | 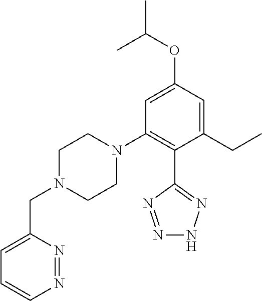 [SMILES: CCc1cc(OC(C)C)cc(N2CCN(Cc3cccnn3)CC2)c1-c1nn[nH]n1] | 3-[[4-[3-ethyl-5- isopropoxy-2-(2H- tetrazol-5-yl)phen- yl]piperazin-1-yl]- methyl]pyridazine | D | 408.50 | 409.3 |
| A-160 | 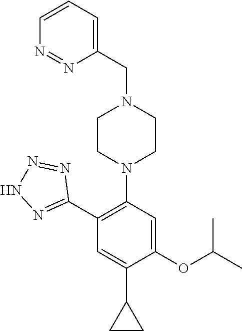 [SMILES: CC(C)Oc1cc(N2CCN(Cc3cccnn3)CC2)c(-c2nn[nH]n2)cc1C1CC1] | 3-[[4-[4-cycloprop- yl-5-isopropoxy-2- (2H-tetrazol-5-yl)- phenyl]piperazin- 1-yl]methyl]pyrid- azine | D | 420.51 | 421.3 |
| A-161 | 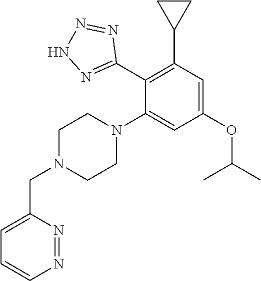 [SMILES: CC(C)Oc1cc(C2CC2)c(-c2nn[nH]n2)c(N2CCN(Cc3cccnn3)CC2)c1] | 3-[[4-[3-cycloprop- yl-5-isopropoxy-2- (2H-tetrazol-5-yl)- phenyl]piperazin- 1-yl]methyl]pyrid- azine | D | 420.51 | 421.3 |
| A-162 | 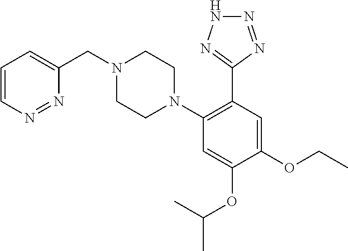 [SMILES: CCOc1cc(-c2nn[nH]n2)c(N2CCN(Cc3cccnn3)CC2)cc1OC(C)C] | 3-[[4-[4-ethoxy-5- isopropoxy-2-(2H- tetrazol-5-yl)phen- yl]piperazin-1-yl]- methyl]pyridazine | D | 424.50 | 425.3 |
| A-163 | 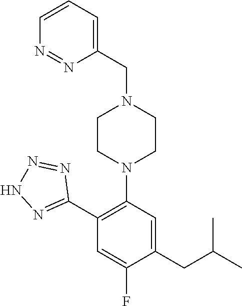 [SMILES: CC(C)Cc1cc(N2CCN(Cc3cccnn3)CC2)c(-c2nn[nH]n2)cc1F] | 3-[[4-[4-fluoro-5- isobutyl-2-(2H- tetrazol-5-yl)phen- yl]piperazin-1-yl]- methyl]pyridazine | D | 396.46 | 397.2 |
| A-164 | 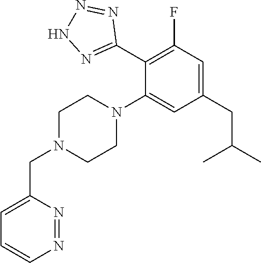 [SMILES: CC(C)Cc1cc(F)c(-c2nn[nH]n2)c(N2CCN(Cc3cccnn3)CC2)c1] | 3-[[4-[3-fluoro-5- isobutyl-2-(2H- tetrazol-5-yl)phen- yl]piperazin-1-yl]- methyl]pyridazine | D | 396.46 | 397.3 |
| A-165 | 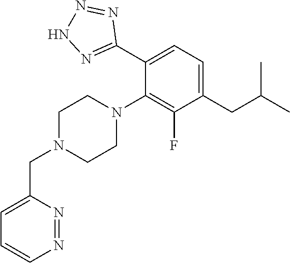 [SMILES: CC(C)Cc1ccc(-c2nn[nH]n2)c(N2CCN(Cc3cccnn3)CC2)c1F] | 3-[[4-[2-fluoro-3- isobutyl-6-(2H- tetrazol-5-yl)phen- yl]piperazin-1-yl]- methyl]pyridazine | D | 396.46 | 397.2 |
| A-166 | 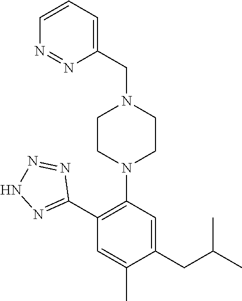 [SMILES: Cc1cc(-c2nn[nH]n2)c(N2CCN(Cc3cccnn3)CC2)cc1CC(C)C] | 3-[[4-[5-isobutyl- 4-methyl-2-(2H- tetrazol-5-yl)phen- yl]piperazin-1-yl]- methyl]pyridazine | D | 392.50 | 393.3 |
| A-167 | 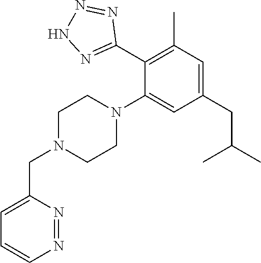 [SMILES: Cc1cc(CC(C)C)cc(N2CCN(Cc3cccnn3)CC2)c1-c1nn[nH]n1] | 3-[[4-[5-isobutyl-3- methyl-2-(2H- tetrazol-5-yl)phen- yl]piperazin-1-yl]- methyl]pyridazine | D | 392.50 | 393.2 |
| A-168 | 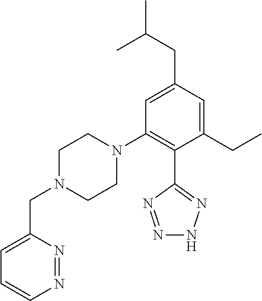 [SMILES: CCc1cc(CC(C)C)cc(N2CCN(Cc3cccnn3)CC2)c1-c1nn[nH]n1] | 3-[4-[3-ethyl-5- isobutyl-2-(2H- tetrazol-5-yl)phen- yl]piperazin-1-yl]- methyl]pyridazine | D | 406.53 | 407.2 |
| A-169 | 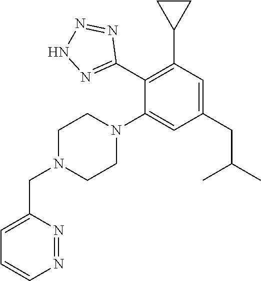 [SMILES: CC(C)Cc1cc(C2CC2)c(-c2nn[nH]n2)c(N2CCN(Cc3cccnn3)CC2)c1] | 3-[[4-[3-cycloprop- yl-5-isobutyl-2-(2H- tetrazol-5-yl)phen- yl]piperazin-1-yl]- methyl]pyridazine | D | 418.54 | 419.2 |
| A-170 | 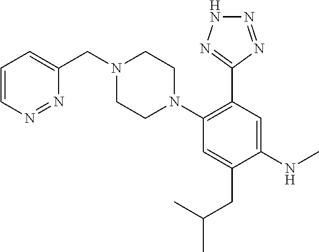 [SMILES: CNc1cc(-c2nn[nH]n2)c(N2CCN(Cc3cccnn3)CC2)cc1CC(C)C] | 2-isobutyl-N-meth- yl-4-[4-(pyridazin- 3-ylmethyl)piper- azin-1-yl]-5-(2H- tetrazol-5-yl)aniline | D | 407.52 | 408.2 |
| A-171 | 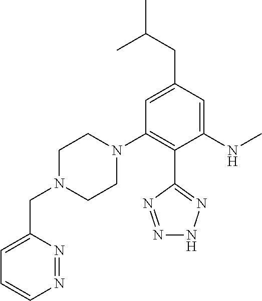 [SMILES: CNc1cc(CC(C)C)cc(N2CCN(Cc3cccnn3)CC2)c1-c1nn[nH]n1] | 5-isobutyl-N-meth- yl-3-[4-(pyridazin- 3-ylmethyl)piper- azin-1-yl]-2-(2H- tetrazol-5-yl)aniline | D | 407.52 | 408.2 |
| A-172 | 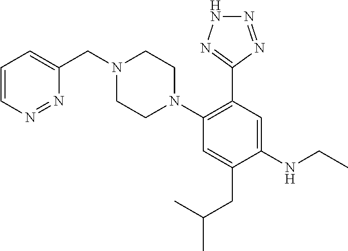 [SMILES: CCNc1cc(-c2nn[nH]n2)c(N2CCN(Cc3cccnn3)CC2)cc1CC(C)C] | N-ethyl-2-isobutyl- 4-[4-(pyridazin-3- ylmethyl)piperazin- 1-yl]-5-(2H-tetrazol- 5-yl)aniline | D | 421.54 | 422.3 |
| A-173 | 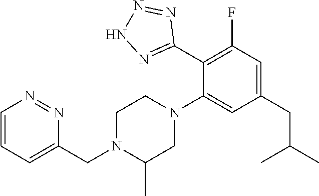 [SMILES: CC(C)Cc1cc(F)c(-c2nn[nH]n2)c(N2CCN(Cc3cccnn3)C(C)C2)c1] | 3-[[4-[3-fluoro-5- isobutyl-2-(2H- tetrazol-5-yl)phen- yl]-2-methyl- piperazin-1-yl]- methyl]pyridazine | G | 410.49 | 411.2 |
| A-174 | 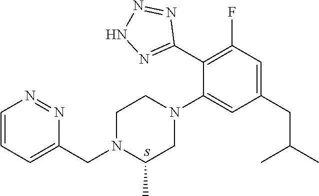 | 3-[[(2S)-4-[3-fluoro- 5-isobutyl-2-(2H- tetrazol-5-yl)phen- yl]-2-methyl- piperazin-1-yl]- methyl]pyridazine | G | 410.49 | 411.3 |
| A-175 | 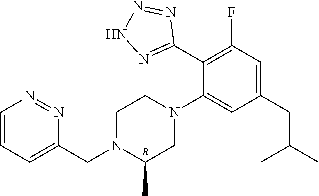 | 3-[[(2R)-4-[3- fluoro-5-isobutyl- 2-(2H-tetrazol-5- yl)phenyl]-2-meth- yl-piperazin-1-yl]- methyl]pyridazine | G | 410.49 | 411.3 |
| A-176 | 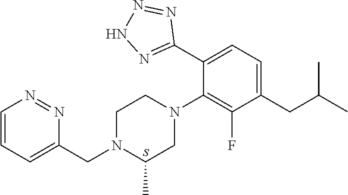 | 3-[[(2S)-4-[2-fluoro- 3-isobutyl-6-(2H- tetrazol-5-yl)phen- yl]-2-methyl- piperazin-1-yl]meth- yl]pyridazine | G | 410.49 | 411.2 |
| A-177 | 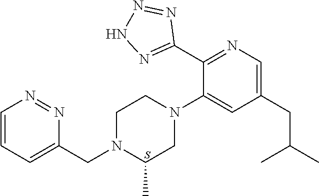 | 3-[[(2S)-4-[5- isobutyl-2-(2H- tetrazol-5-yl)-3- pyridyl]-2-methyl- piperazin-1-yl]- methyl]pyridazine | G | 393.49 | 394.2 |
| A-178 | 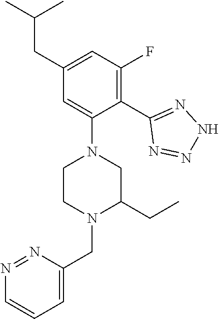 [SMILES: CCC1CN(c2cc(CC(C)C)cc(F)c2-c2nn[nH]n2)CCN1Cc1cccnn1] | 3-[[2-ethyl-4-[3- fluoro-5-isobutyl-2- (2H-tetrazol-5-yl)- phenyl]piperazin- 1-yl]methyl]pyrid- azine | G | 424.52 | 425.3 |
| A-179 | 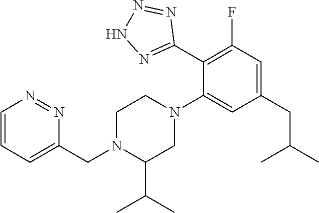 [SMILES: CC(C)Cc1cc(F)c(-c2nn[nH]n2)c(N2CCN(Cc3cccnn3)C(C(C)C)C2)c1] | 3-[[4-[3-fluoro-5- isobutyl-2-(2H- tetrazol-5-yl)phen- yl]-2-isopropyl- piperazin-1-yl]- methyl]pyridazine | G | 438.54 | 439.2 |
| A-180 | 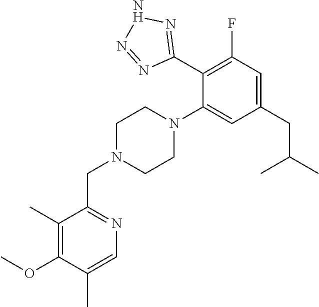 [SMILES: COc1c(C)cnc(CN2CCN(c3cc(CC(C)C)cc(F)c3-c3nn[nH]n3)CC2)c1C] | 3-[[4-[3-fluoro-5- isobutyl-2-(2H- tetrazol-5-yl)phen- yl]-3-methyl- piperazin-1-yl]- methyl]pyridazine | E | 410.49 | 411.3 |
| A-181 | 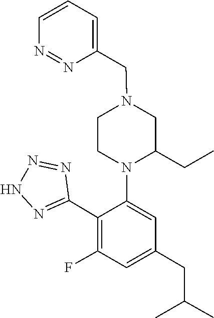 [SMILES: CCC1CN(Cc2cccnn2)CCN1c1cc(CC(C)C)cc(F)c1-c1nn[nH]n1] | 3-[[3-ethyl-4-[3- fluoro-5-isobutyl-2- (2H-tetrazol-5-yl)- phenyl]piperazin- 1-yl]methyl]pyrid- azine | F | 424.52 | 425.3 |
| A-182 | 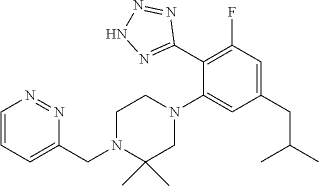 [SMILES: CC(C)Cc1cc(F)c(-c2nn[nH]n2)c(N2CCN(Cc3cccnn3)C(C)(C)C2)c1] | 3-[[4-[3-fluoro-5- isobutyl-2-(2H- tetrazol-5-yl)phen- yl]-2,2-dimethyl- piperazin-1-yl]- methyl]pyridazine | G | 424.52 | 425.1 |
| A-183 | 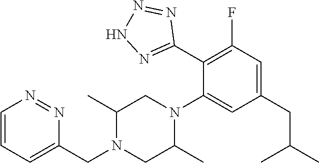 [SMILES: CC(C)Cc1cc(F)c(-c2nn[nH]n2)c(N2CC(C)N(Cc3cccnn3)CC2C)c1] | 3-[[4-[3-fluoro-5- isobutyl-2-(2H- tetrazol-5-yl)phen- yl]-2,5-dimethyl- piperazin-1-yl]- methyl]pyridazine | F | 424.52 | 425.2 |
| A-184 | 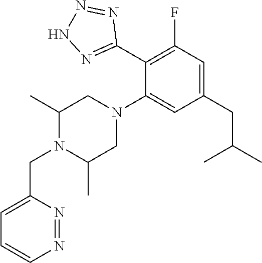 [SMILES: CC(C)Cc1cc(F)c(-c2nn[nH]n2)c(N2CC(C)N(Cc3cccnn3)C(C)C2)c1] | 3-[[4-[3-fluoro-5- isobutyl-2-(2H- tetrazol-5-yl)phen- yl]-2,6-dimethyl- piperazin-1-yl]- methyl]pyridazine | F | 424.52 | 425.2 |
| A-185 | 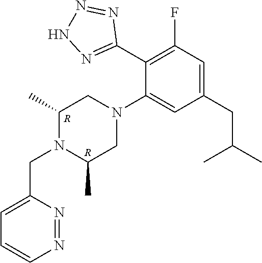 | 3-[[(2R,6R)-4-[3- fluoro-5-isobutyl-2- (2H-tetrazol-5-yl)- phenyl]-2,6-dimeth- yl-piperazin-1-yl]- methyl]pyridazine | G | 424.52 | 425.2 |
| A-186 | 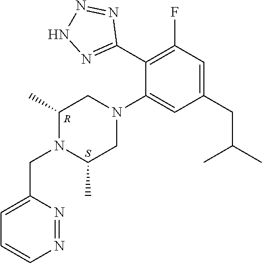 | 3-[[(2S,6R)-4-[3- fluoro-5-isobutyl-2- (2H-tetrazol-5-yl)- phenyl]-2,6-dimeth- yl-piperazin-1-yl]- methyl]pyridazine | G | 424.52 | 425.2 |
| A-187 | 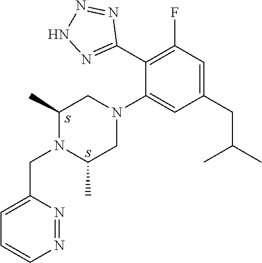 | 3-[[(2S,6S)-4-[3- fluoro-5-isobutyl-2- (2H-tetrazol-5-yl)- phenyl]-2,6-dimeth- yl-piperazin-1-yl]- methyl]pyridazine | G | 424.52 | 425.2 |
| A-188 | 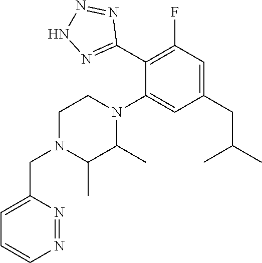 [SMILES: CC(C)Cc1cc(F)c(-c2nn[nH]n2)c(N2CCN(Cc3cccnn3)C(C)C2C)c1] | 3-[[4-[3-fluoro-5- isobutyl-2-(2H- tetrazol-5-yl)phen- yl]-2,3-dimethyl- piperazin-1-yl]- methyl]pyridazine | E | 424.52 | 425.2 |
| A-189 | 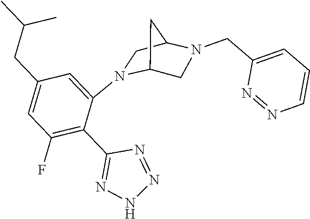 [SMILES: CC(C)Cc1cc(F)c(-c2nn[nH]n2)c(N2CC3CC2CN3Cc2cccnn2)c1] | 2-[3-fluoro-5- isobutyl-2-(2H- tetrazol-5-yl)phen- yl]-5-(pyridazin-3- ylmethyl)-2,5-di- azabicyclo[2.2.1]- heptane | E | 408.48 | 409.2 |
| A-190 | 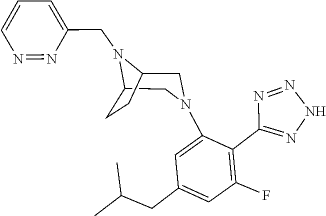 [SMILES: CC(C)Cc1cc(F)c(-c2nn[nH]n2)c(N2CC3CCC(C2)N3Cc2cccnn2)c1] | 3-[3-fluoro-5- isobutyl-2-(2H- tetrazol-5-yl)phen- yl]-8-(pyridazin-3- ylmethyl)-3,8-diaza- bicyclo[3.2.1]octane | F | 422.50 | 423.0 |
| A-191 | 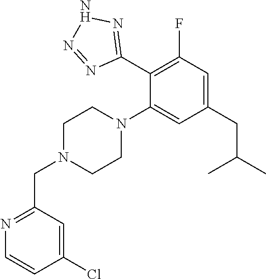 [SMILES: CC(C)Cc1cc(F)c(-c2nn[nH]n2)c(N2CCN(Cc3cc(Cl)ccn3)CC2)c1] | 1-[(4-chloro-2- pyridyl)methyl]-4- [3-fluoro-5-isobutyl- 2-(2H-tetrazol-5- yl)phenyl]piperazine | A | 429.92 | 430.3 432.3 |
| A-192 | 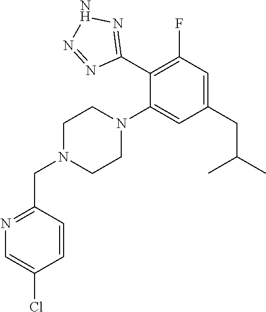 [SMILES: CC(C)Cc1cc(F)c(-c2nn[nH]n2)c(N2CCN(Cc3ccc(Cl)cn3)CC2)c1] | 1-[(5-chloro-2- pyridyl)methyl]-4- [3-fluoro-5-isobutyl- 2-(2H-tetrazol-5- yl)phenyl]piperazine | A | 429.92 | 430.3 432.3 |
| A-193 | 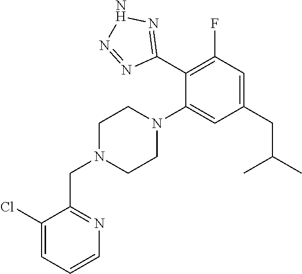 [SMILES: CC(C)Cc1cc(F)c(-c2nn[nH]n2)c(N2CCN(Cc3ncccc3Cl)CC2)c1] | 1-[(3-chloro-2- pyridyl)methyl]-4- [3-fluoro-5-isobutyl- 2-(2H-tetrazol-5- yl)phenyl]piperazine | A | 429.92 | 430.3 432.3 |
| A-194 | 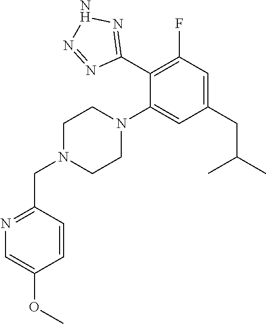 [SMILES: COc1ccc(CN2CCN(c3cc(CC(C)C)cc(F)c3-c3nn[nH]n3)CC2)nc1] | 1-[3-fluoro-5- isobutyl-2-(2H- tetrazol-5-yl)phen- yl]-4-[(5-methoxy- 2-pyridyl)meth- yl]piperazine | A | 425.50 | 426.3 |
| A-195 | 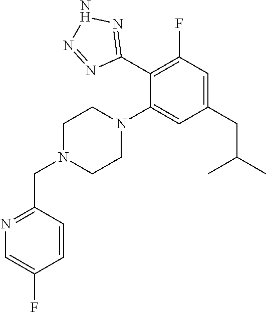 [SMILES: CC(C)Cc1cc(F)c(-c2nn[nH]n2)c(N2CCN(Cc3ccc(F)cn3)CC2)c1] | 1-[3-fluoro-5- isobutyl-2-(2H- tetrazol-5-yl)phen- yl]-4-[(5-fluoro-2- pyridyl)methyl]- piperazine | A | 413.47 | 414.3 |
| A-196 | 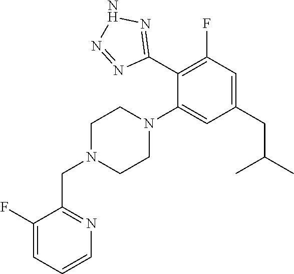 [SMILES: CC(C)Cc1cc(F)c(-c2nn[nH]n2)c(N2CCN(Cc3ncccc3F)CC2)c1] | 1-[3-fluoro-5- isobutyl-2-(2H- tetrazol-5-yl)phen- yl]-4-[(3-fluoro-2- pyridyl)methyl]- piperazine | A | 413.47 | 414.3 |
| A-197 | 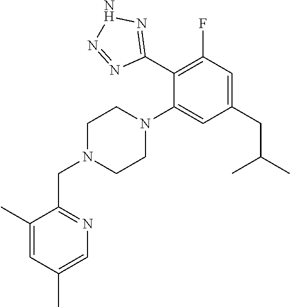 [SMILES: Cc1cnc(CN2CCN(c3cc(CC(C)C)cc(F)c3-c3nn[nH]n3)CC2)c(C)c1] | 1-[(3,5-dimethyl-2- pyridyl)methyl]-4- [3-fluoro-5-isobutyl- 2-(2H-tetrazol-5- yl)phenyl]piperazine | A | 423.53 | 424.4 |
| A-198 | 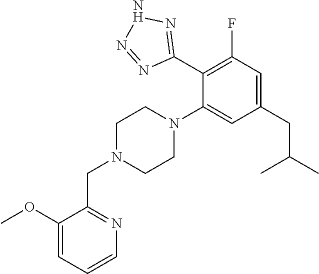 [SMILES: COc1cccnc1CN1CCN(c2cc(CC(C)C)cc(F)c2-c2nn[nH]n2)CC1] | 1-[3-fluoro-5- isobutyl-2-(2H- tetrazol-5-yl)phen- yl]-4-[(3-methoxy- 2-pyridyl)meth- yl]piperazine | A | 425.50 | 426.3 |
| A-199 | 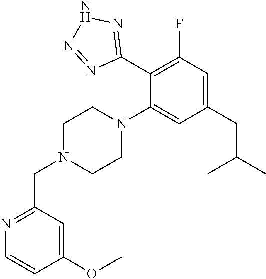 [SMILES: COc1ccnc(CN2CCN(c3cc(CC(C)C)cc(F)c3-c3nn[nH]n3)CC2)c1] | 1-[3-fluoro-5- isobutyl-2-(2H- tetrazol-5-yl)phen- yl]-4-[(4-methoxy- 2-pyridyl)methyl]- piperazine | A | 425.50 | 426.3 |
| A-200 | 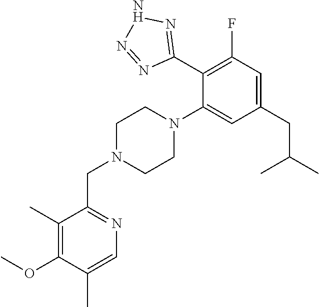 [SMILES: COc1c(C)cnc(CN2CCN(c3cc(CC(C)C)cc(F)c3-c3nn[nH]n3)CC2)c1C] | 1-[3-fluoro-5- isobutyl-2-(2H- tetrazol-5-yl)phen- yl]-4-[(4-methoxy- 3,5-dimethyl-2- pyridyl)methyl]- piperazine | A | 453.56 | 454.3 |
| A-201 | 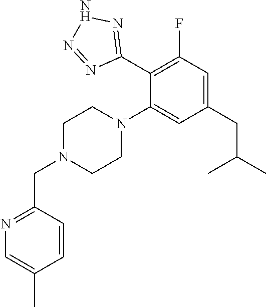 [SMILES: Cc1ccc(CN2CCN(c3cc(CC(C)C)cc(F)c3-c3nn[nH]n3)CC2)nc1] | 1-[3-fluoro-5- isobutyl-2-(2H- tetrazol-5-yl)phen- yl]-4-[(5-methyl-2- pyridyl)methyl]- piperazine | A | 409.50 | 410.3 |
| A-202 | 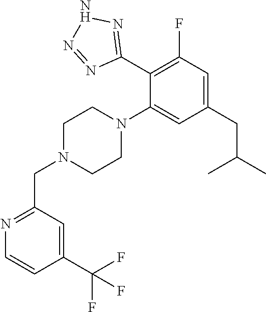 [SMILES: CC(C)Cc1cc(F)c(-c2nn[nH]n2)c(N2CCN(Cc3cc(C(F)(F)F)ccn3)CC2)c1] | 1-[3-fluoro-5- isobutyl-2-(2H- tetrazol-5-yl)phen- yl]-4-[[4-(trifluoro- methyl)-2-pyridyl]- methyl]piperazine | A | 463.47 | 464.2 |
| A-203 | 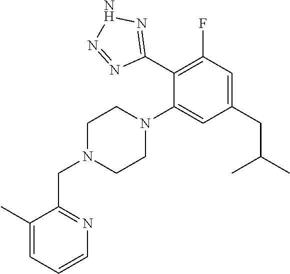 [SMILES: Cc1cccnc1CN1CCN(c2cc(CC(C)C)cc(F)c2-c2nn[nH]n2)CC1] | 1-[3-fluoro-5- isobutyl-2-(2H- tetrazol-5-yl)phen- yl]-4-[(3-methyl-2- pyridyl)methyl]- piperazine | A | 409.50 | 410.3 |
| A-204 | 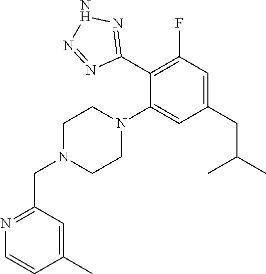 [SMILES: Cc1ccnc(CN2CCN(c3cc(CC(C)C)cc(F)c3-c3nn[nH]n3)CC2)c1] | 1-[3-fluoro-5- isobutyl-2-(2H- tetrazol-5-yl)phen- yl]-4-[(4-methyl-2- pyridyl)methyl]- piperazine | A | 409.50 | 410.3 |
| A-205 | 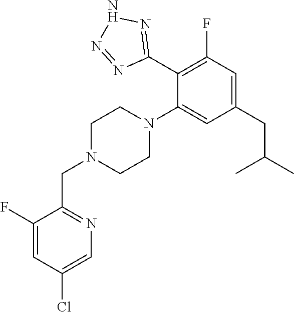 [SMILES: CC(C)Cc1cc(F)c(-c2nn[nH]n2)c(N2CCN(Cc3ncc(Cl)cc3F)CC2)c1] | 1-[(5-chloro-3- fluoro-2-pyridyl)- methyl]-4-[3-fluoro- 5-isobutyl-2-(2H- tetrazol-5-yl)phen- yl]piperazine | A | 447.91 | 448.2 450.2 |
| A-206 | 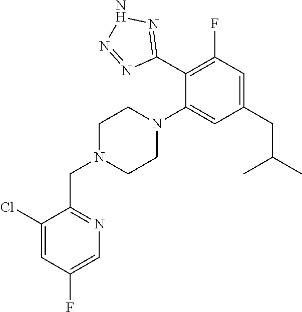 [SMILES: CC(C)Cc1cc(F)c(-c2nn[nH]n2)c(N2CCN(Cc3ncc(F)cc3Cl)CC2)c1] | 1-[(3-chloro-5- fluoro-2-pyridyl)- methyl]-4-[3-fluoro- 5-isobutyl-2-(2H- tetrazol-5-yl)phen- yl]piperazine | A | 447.91 | 448.2 450.2 |
| A-207 | 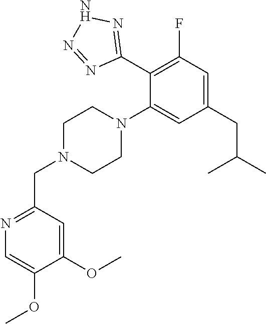 [SMILES: COc1cnc(CN2CCN(c3cc(CC(C)C)cc(F)c3-c3nn[nH]n3)CC2)cc1OC] | 1-[(4,5-dimethoxy- 2-pyridyl)methyl]- 4-[3-fluoro-5-iso- butyl-2-(2H- tetrazol-5-yl)phen- yl]piperazine | A | 455.53 | 456.3 |
| A-208 | 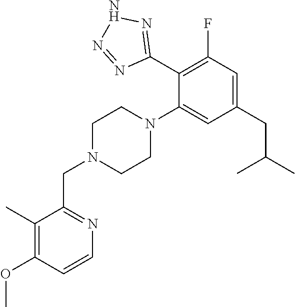 [SMILES: COc1ccnc(CN2CCN(c3cc(CC(C)C)cc(F)c3-c3nn[nH]n3)CC2)c1C] | 1-[3-fluoro-5- isobutyl-2-(2H- tetrazol-5-yl)phen- yl]-4-[(4-methoxy- 3-methyl-2-pyrid- yl)methyl]piper- azine | A | 439.53 | 440.3 |
| A-209 | 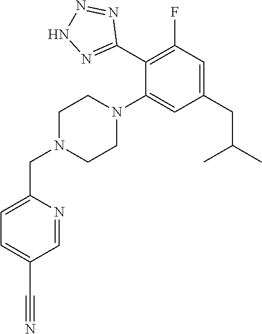 [SMILES: CC(C)Cc1cc(F)c(-c2nn[nH]n2)c(N2CCN(Cc3ccc(C#N)cn3)CC2)c1] | 6-[[4-[3-fluoro-5- isobutyl-2-(2H- tetrazol-5-yl)phen- yl]piperazin-1-yl]- methyl]pyridine-3- carbonitrile | X | 420.49 | 421.2 |
| A-210 | 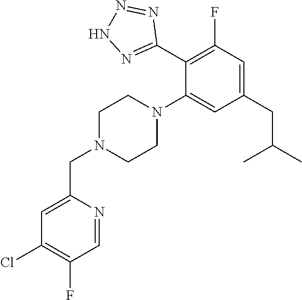 [SMILES: CC(C)Cc1cc(F)c(-c2nn[nH]n2)c(N2CCN(Cc3cc(Cl)c(F)cn3)CC2)c1] | 1-[(4-chloro-5- fluoro-2-pyridyl)- methyl]-4-[3-fluoro- 5-isobutyl-2-(2H- tetrazol-5-yl)phen- yl]piperazine | A | 447.91 | 448.1 450.1 |
| A-211 | 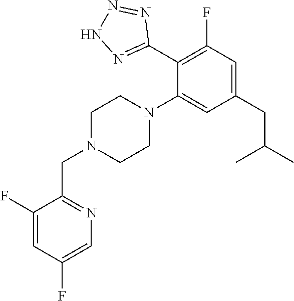 [SMILES: CC(C)Cc1cc(F)c(-c2nn[nH]n2)c(N2CCN(Cc3ncc(F)cc3F)CC2)c1] | 1-[(3,5-difluoro-2- pyridyl)methyl]-4- [3-fluoro-5-isobutyl- 2-(2H-tetrazol-5-yl)- phenyl]piperazine | A | 431.46 | 432.1 |
| A-212 | 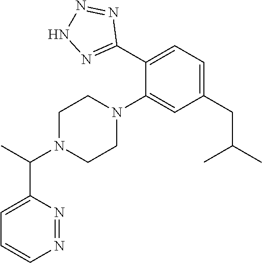 [SMILES: CC(C)Cc1ccc(-c2nn[nH]n2)c(N2CCN(C(C)c3cccnn3)CC2)c1] | 3-[1-[4-[5-isobutyl- 2-(2H-tetrazol-5-yl)- phenyl]piperazin- 1-yl]ethyl]pyrid- azine | U | 392.50 | 393.2 |
| A-213 | 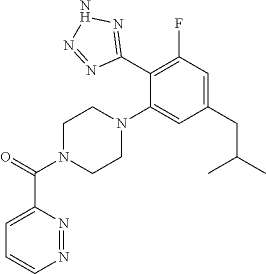 [SMILES: CC(C)Cc1cc(F)c(-c2nn[nH]n2)c(N2CCN(C(=O)c3cccnn3)CC2)c1] | [4-[3-fluoro-5- isobutyl-2-(2H- tetrazol-5-yl)phen- yl]piperazin-1-yl]- pyridazin-3-yl- methanone | X | 410.45 | 411.3 |
| A-214 | 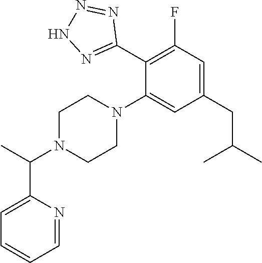 [SMILES: CC(C)Cc1cc(F)c(-c2nn[nH]n2)c(N2CCN(C(C)c3ccccn3)CC2)c1] | 1-[3-fluoro-5- isobutyl-2-(2H- tetrazol-5-yl)phen- yl]-4-[1-(2-pyridyl)- ethyl]piperazine | A | 409.50 | 410.2 |
| A-215 | 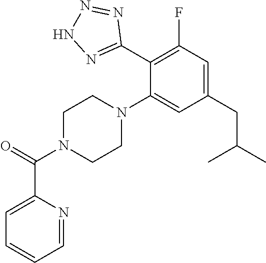 [SMILES: CC(C)Cc1cc(F)c(-c2nn[nH]n2)c(N2CCN(C(=O)c3ccccn3)CC2)c1] | [4-[3-fluoro-5- isobutyl-2-(2H- tetrazol-5-yl)phen- yl]piperazin-1-yl]- (2-pyridyl)meth- anone | Described above | 409.46 | 410.2 |
| A-216 | 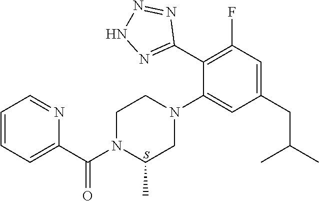 | [(2S)-4-[3-fluoro-5- isobutyl-2-(2H- tetrazol-5-yl)phen- yl]-2-methyl- piperazin-1-yl]-(2- pyridyl)methanone | Described above | 423.49 | 424.1 |
| A-217 | 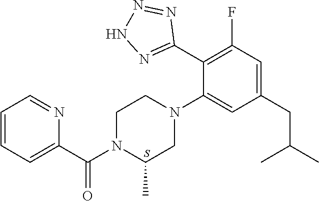 | (2S)-4-[3-fluoro-5- isobutyl-2-(2H- tetrazol-5-yl)phen- yl]-2-methyl-1-(2- pyridylmethyl)piper- azine | AC | 409.50 | 410.2 |
| A-218 | 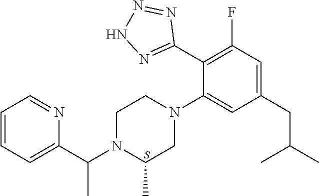 | (2S)-4-[3-fluoro-5- isobutyl-2-(2H- tetrazol-5-yl)phen- yl]-2-methyl-1-[1- (2-pyridyl)ethyl]- piperazine | AC | 423.53 | 424.1 |
| A-219 | 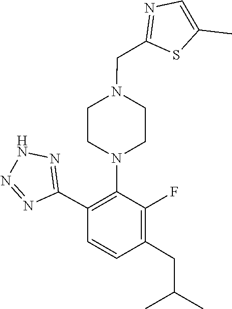 [SMILES: Cc1cnc(CN2CCN(c3c(-c4nn[nH]n4)ccc(CC(C)C)c3F)CC2)s1] | 2-[[4-[2-fluoro-3- isobutyl-6-(2H- tetrazol-5-yl)phen- yl]piperazin-1-yl]- methyl]-5-methyl- thiazole | AB | 415.53 | 416.3 |
| A-220 | 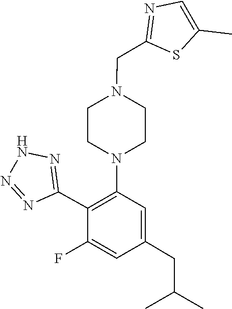 [SMILES: Cc1cnc(CN2CCN(c3cc(CC(C)C)cc(F)c3-c3nn[nH]n3)CC2)s1] | 2-[[4-[3-fluoro-5- isobutyl-2-(2H- tetrazol-5-yl)phen- yl]piperazin-1-yl]- methyl]-5-methyl- thiazole | AB | 415.53 | 416.3 |
| A-221 | 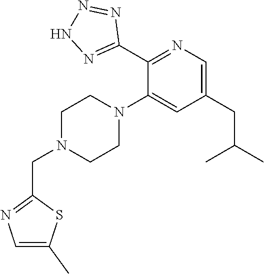 [SMILES: Cc1cnc(CN2CCN(c3cc(CC(C)C)cnc3-c3nn[nH]n3)CC2)s1] | 2-[[4-[5-isobutyl-2- (2H-tetrazol-5-yl)- 3-pyridyl]piperazin- 1-yl]methyl]-5- methyl-thiazole | AB | 398.53 | 399.2 |
| A-222 | 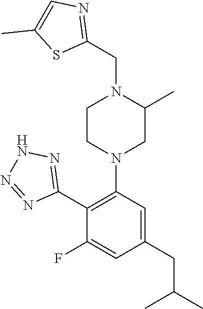 [SMILES: Cc1cnc(CN2CCN(c3cc(CC(C)C)cc(F)c3-c3nn[nH]n3)CC2C)s1] | 2-[[4-[3-fluoro-5- isobutyl-2-(2H- tetrazol-5-yl)phen- yl]-2-methyl- piperazin-1-yl]- methyl]-5-methyl- thiazole | AC | 429.56 | 430.3 |
| A-223 | 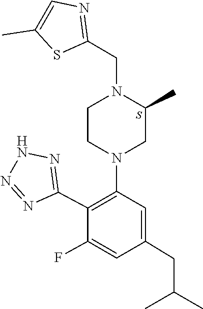 | 2-[[(2S)-4-[3-fluoro- 5-isobutyl-2-(2H- tetrazol-5-yl)phen- yl]-2-methyl-piper- azin-1-yl]methyl]- 5-methyl-thiazole | AC | 429.55 | 430.3 |
| A-224 | 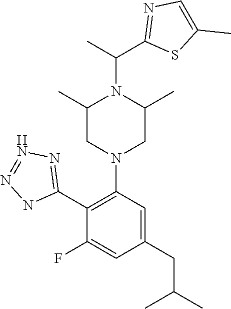 | 2-[[4-[3-fluoro-5- isobutyl-2-(2H- tetrazol-5-yl)phen- yl]-2,6-dimethyl- piperazin-1-yl]- methyl]-5-methyl- thiazole | AD | 443.58 | 444.3 |
| A-225 | 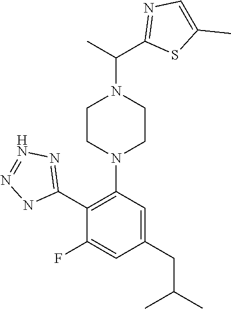 | 2-[1-[4-[3-fluoro-5- isobutyl-2-(2H- tetrazol-5-yl)phen- yl]piperazin-1-yl]- ethyl]-5-methyl- thiazole | AB | 429.56 | 430.3 |
| A-226 | 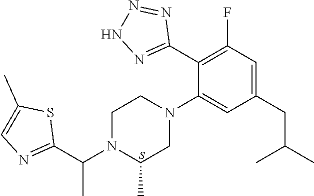 | 2-[1-[(2S)-4-[3- fluoro-5-isobutyl-2- (2H-tetrazol-5-yl)- phenyl]-2-methyl- piperazin-1-yl]- ethyl]-5-methyl- thiazole | AC | 443.58 | 444.3 |
| A-227 | 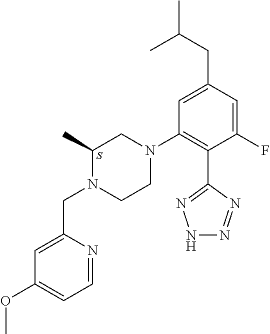 | (2S)-4-[3-fluoro-5- isobutyl-2-(2H- tetrazol-5-yl)phen- yl]-1-[(4-methoxy- 2-pyridyl)methyl]-2- methyl-piperazine | AC | 439.53 | 440.1 |
| A-228 | 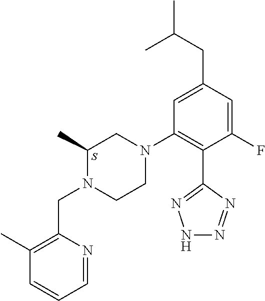 | (2S)-4-[3-fluoro-5- isobutyl-2-(2H- tetrazol-5-yl)phen- yl]-2-methyl-1-[(3- methyl-2-pyridyl)- methyl]piperazine | AC | 423.53 | 424.2 |
| A-229 | 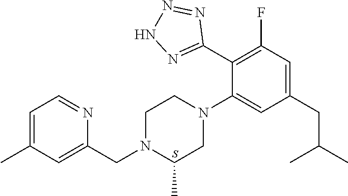 | (2S)-4-[3-fluoro-5- isobutyl-2-(2H- tetrazol-5-yl)phen- yl]-2-methyl-1-[(4- methyl-2-pyridyl)- methyl]piperazine | AC | 423.53 | 424.2 |
| A-230 | 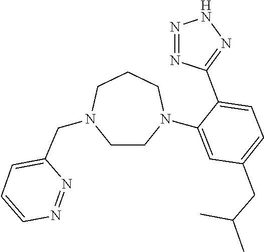 [SMILES: CC(C)Cc1ccc(-c2nn[nH]n2)c(N2CCCN(Cc3cccnn3)CC2)c1] | 1-[5-isobutyl-2- (2H-tetrazol-5-yl)- phenyl]-4-(pyrid- azin-3-ylmethyl)- 1,4-diazepane | D | 392.50 | 393.2 |
| A-231 | 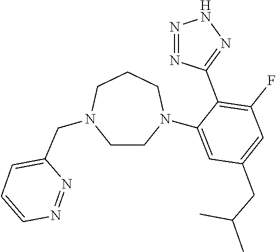 [SMILES: CC(C)Cc1cc(F)c(-c2nn[nH]n2)c(N2CCCN(Cc3cccnn3)CC2)c1] | 1-[3-fluoro-5- isobutyl-2-(2H- tetrazol-5-yl)phen- yl]-4-(pyridazin-3- ylmethyl)-1,4- diazepane | G | 410.49 | 411.2 |
| A-232 | 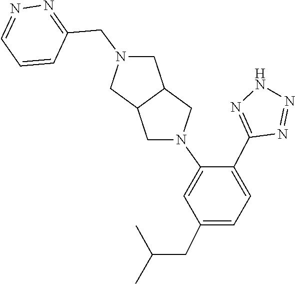 [SMILES: CC(C)Cc1ccc(-c2nn[nH]n2)c(N2CC3CN(Cc4cccnn4)CC3C2)c1] | 5-[5-isobutyl-2- (2H-tetrazol-5-yl)- phenyl]-2-(pyrid- azin-3-ylmethyl)- 1,3,3a,4,6,6a- hexahydropyrrolo- [3,4-c]pyrrole | D | 404.51 | 405.2 |
| A-233 | 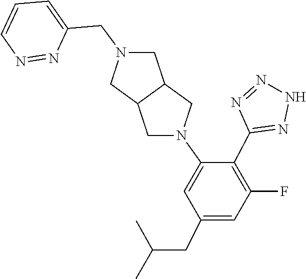 [SMILES: CC(C)Cc1cc(F)c(-c2nn[nH]n2)c(N2CC3CN(Cc4cccnn4)CC3C2)c1] | 5-[3-fluoro-5- isobutyl-2-(2H- tetrazol-5-yl)phen- yl]-2-(pyridazin- 3-ylmethyl)- 1,3,3a,4,6,6a- hexahydropyrrolo- [3,4-c]pyrrole | G | 422.50 | 423.2 |
| A-234 | 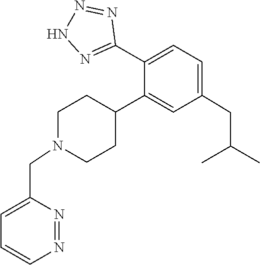 [SMILES: CC(C)Cc1ccc(-c2nn[nH]n2)c(C2CCN(Cc3cccnn3)CC2)c1] | 3-[[4-[5-isobutyl-2- (2H-tetrazol-5-yl)- phenyl]-1-piperid- yl]methyl]pyrid- azine | D | 377.49 | 378.1 |
| A-235 | 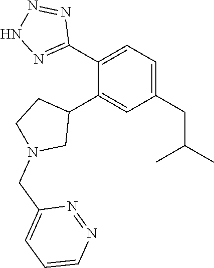 [SMILES: CC(C)Cc1ccc(-c2nn[nH]n2)c(C2CCN(Cc3cccnn3)C2)c1] | 3-[[3-[5-isobutyl-2- (2H-tetrazol-5-yl)- phenyl]pyrrolidin- 1-yl]methyl]pyrid- azine | D | 363.46 | 364.1 |
| A-236 | 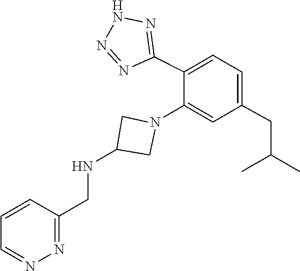 [SMILES: CC(C)Cc1ccc(-c2nn[nH]n2)c(N2CC(NCc3cccnn3)C2)c1] | 1-(5-isobutyl-2- (2H-tetrazol-5-yl)- phenyl)-N-(pyrid- azin-3-ylmethyl)- azetidin-3-amine | W | 364.45 | 365.2 |
| A-237 | 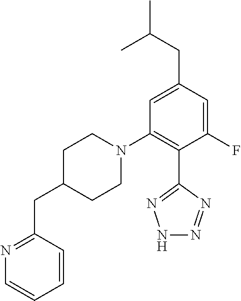 [SMILES: CC(C)Cc1cc(F)c(-c2nn[nH]n2)c(N2CCC(Cc3ccccn3)CC2)c1] | 2-((1-(3-fluoro-5- isobutyl-2-(2H- tetrazol-5-yl)phen- yl)piperidin-4-yl)- methyl)pyridine | AB | 394.49 | 395.2 |
| A-238 | 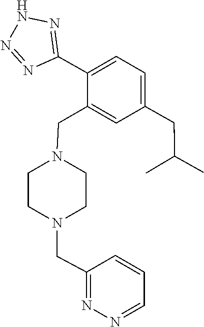 [SMILES: CC(C)Cc1ccc(-c2nn[nH]n2)c(CN2CCN(Cc3cccnn3)CC2)c1] | 3-[[4-[[5-isobutyl- 2-(2H-tetrazol-5- yl)phenyl]methyl]- piperazin-1-yl]- methyl]pyridazine | D | 392.50 | 393.3 |
| A-239 | 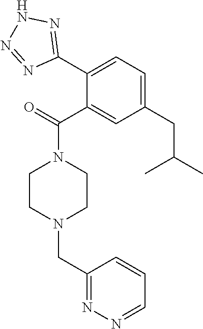 [SMILES: CC(C)Cc1ccc(-c2nn[nH]n2)c(C(=O)N2CCN(Cc3cccnn3)CC2)c1] | (5-isobutyl-2-(2H- tetrazol-5-yl)phen- yl)(4-(pyridazin-3- ylmethyl)piperazin- 1-yl)methanone | Described above | 406.48 | 407.3 |
| A-240 | 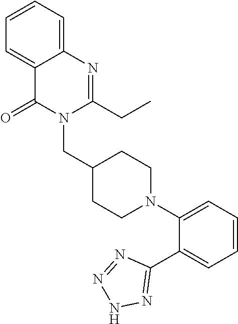 [SMILES: CCc1nc2ccccc2c(=O)n1CC1CCN(c2ccccc2-c2nn[nH]n2)CC1] | 2-ethyl-3-[[1-[2- (2H-tetrazol-5-yl)- phenyl]-4-piperid- yl]methyl]quin- azolin-4-one | AE | 415.49 | 416.2 |
| A-241 | 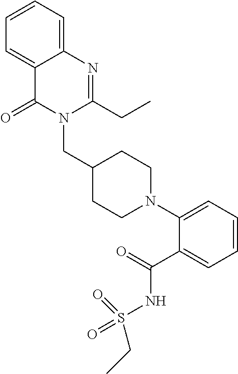 [SMILES: CCc1nc2ccccc2c(=O)n1CC1CCN(c2ccccc2C(=O)NS(=O)(=O)CC)CC1] | 2-[4-[(2-ethyl-4- oxo-quinazolin-3- yl)methyl]-1- piperidyl]-N-ethyl- sulfonyl-benzamide | AE | 482.60 | 483.2 |
| A-242 | 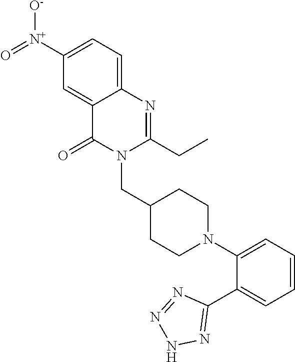 [SMILES: CCc1nc2ccc([N+](=O)[O-])cc2c(=O)n1CC1CCN(c2ccccc2-c2nn[nH]n2)CC1] | 2-ethyl-6-nitro-3- [[1-[2-(2H-tetrazol- 5-yl)phenyl]-4- piperidyl]methyl]- quinazolin-4-one | AF | 460.49 | 461.2 |
| A-243 | 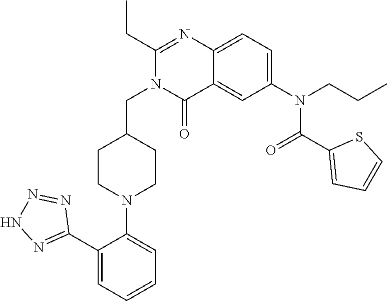 [SMILES: CCCN(C(=O)c1cccs1)c1ccc2nc(CC)n(CC3CCN(c4ccccc4-c4nn[nH]n4)CC3)c(=O)c2c1] | N-[2-ethyl-4-oxo- 3-[[1-[2-(2H- tetrazol-5-yl)phen- yl]-4-piperidyl]- methyl]quinazolin- 6-yl]-N-propyl- thiophene-2-carbox- amide | AF | 582.72 | 583.3 |
| A-244 | 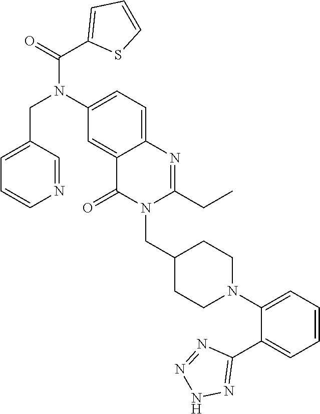 [SMILES: CCc1nc2ccc(N(Cc3cccnc3)C(=O)c3cccs3)cc2c(=O)n1CC1CCN(c2ccccc2-c2nn[nH]n2)CC1] | N-[2-ethyl-4-oxo-3- [1-[2-(2H-tetrazol- 5-yl)phenyl]-4- piperidyl]methyl]- quinazolin-6-yl]-N- (3-pyridylmethyl)- thiophene-2-carbox- amide | AF | 631.75 | 632.3 |
| A-245 | 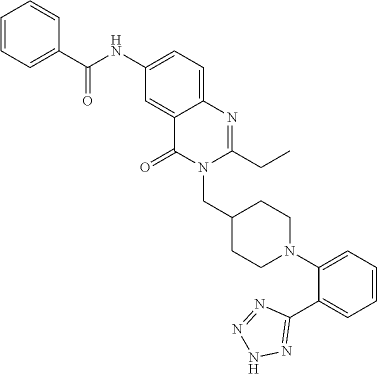 [SMILES: CCc1nc2ccc(NC(=O)c3ccccc3)cc2c(=O)n1CC1CCN(c2ccccc2-c2nn[nH]n2)CC1] | N-[2-ethyl-4-oxo-3- [1-[2-(2H-tetrazol- 5-yl)phenyl]-4- piperidyl]methyl]- quinazolin-6-yl]- benzamide | AF | 534.61 | 353.3 |
| A-246 | 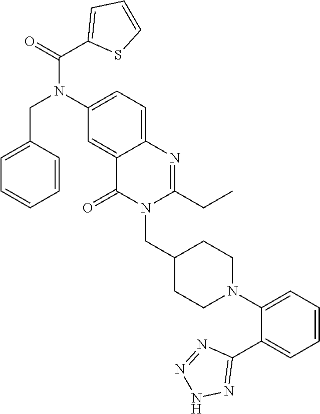 [SMILES: CCc1nc2ccc(N(Cc3ccccc3)C(=O)c3cccs3)cc2c(=O)n1CC1CCN(c2ccccc2-c2nn[nH]n2)CC1] | N-benzyl-N-[2- ethyl-4-oxo-3-[[1- [2-(2H-tetrazol-5- yl)phenyl]-4-piper- idyl]methyl]quin- azolin-6-yl]thio- phene-2-carbox- amide | AF | 630.76 | 631.4 |
| A-247 | 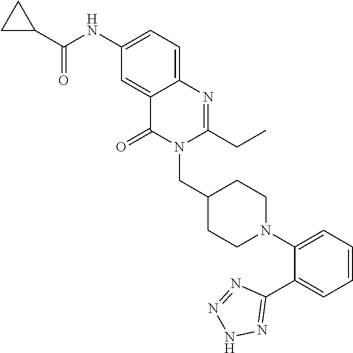 [SMILES: CCc1nc2ccc(NC(=O)C3CC3)cc2c(=O)n1CC1CCN(c2ccccc2-c2nn[nH]n2)CC1] | N-[2-ethyl-4-oxo-3- [[1-[2-(2H-tetrazol- 5-yl)phenyl]-4- piperidyl]methyl]- quinazolin-6-yl]- cyclopropane- carboxamide | AF | 498.58 | 499.3 |
| A-248 | 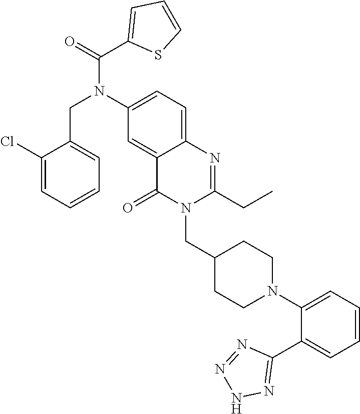 [SMILES: CCc1nc2ccc(N(Cc3ccccc3Cl)C(=O)c3cccs3)cc2c(=O)n1CC1CCN(c2ccccc2-c2nn[nH]n2)CC1] | N-[(2-chlorophen- yl)-methyl]-N-[2- ethyl-4-oxo-3-[[1- [2-(2H-tetrazol-5- yl)phenyl]-4-piper- idyl]methyl]quin- azolin-6-yl]thio- phene-2-carbox- amide | AF | 665.21 | 665.3 667.3 |
| A-249 | 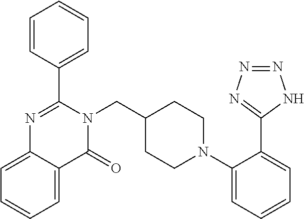 [SMILES: O=c1c2ccccc2nc(-c2ccccc2)n1CC1CCN(c2ccccc2-c2nnn[nH]2)CC1] | 2-phenyl-3-[1-[2- (1H-tetrazol-5-yl)- phenyl]-4-piperid- yl]methyl]quin- azolin-4-one | AG | 463.53 | 464.2 |
| A-250 | 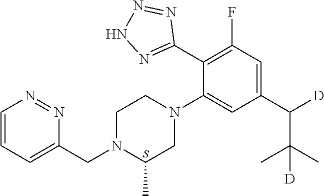 | 3-[[(2S)-4-[5-(1,2- dideuterio-2-meth- yl-propyl)-3-f(luoro- 2-(2H-tetrazol-5- yl)phenyl]-2-meth- yl-piperazin-1-yl]- methyl]pyridazine | G | 412.50 | 413.4 |
| A-251 | 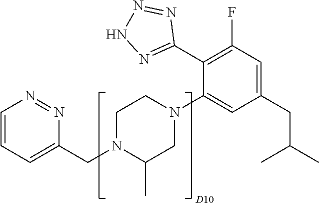 | 2,2,3,3,5,6,6- heptadeuterio-1-[3- fluoro-5-isobutyl-2- (2H-tetrazol-5-yl)- phenyl]-4-(pyrid- azin-3-ylmethyl)- 5-(trideuteriometh- yl)piperazine | E | 420.55 | 421.0 |

TABLE 2

| Compound | |
|---|---|
| number | (δ) NMR data |
| A-01 | ¹H NMR δ (ppm) (DMSO-d₆): 7.79-7.49 (2H, m, ArH), 7.48-7.40 (4H, m, |
| | ArH), 7.26 (1H, t, ArH), 7.06 (1H, t, ArH), 3.90 (2H, m, CH₂), 3.58 (4H, |
| | s, CH₂), 2.65 (4H, s, CH₂). |
| A-02 | ¹H NMR δ (ppm) (DMSO-d₆): 7.83 (1H, d, ArH), 7.77 (1H, d, ArH), 7.52 |
| | (2H, t, ArH), 7.46 (1H, d, ArH), 7.37 (1H, d, ArH), 7.27 (1H, t, ArH), |
| | 7.08 (1H, t, ArH), 4.03 (2H, s, CH₂), 3.69 (4H, s, CH₂), 3.39 (2H, t, CH₂), |
| | 2.85 (4H, s, CH₂), 1.23 (3H, t, CH₃). |
| A-03 | ¹H NMR δ (ppm) (CHCl₃-d): 8.32 (1H, d, ArH), 7.63-7.57 (3H, m, ArH), |
| | 7.52 (1H, t, ArH), 7.42 (1H, d, ArH), 7.33 (1H, t, ArH), 7.13 (1H, t, ArH), |
| | 3.85 (4H, br. s, CH₂), 3.83 (2H, s, NCH₂), 2.91 (4H, br. s, CH₂). |
| A-04 | ¹H NMR δ (ppm) (CHCl₃-d): 14.79 (1H, s, NH), 8.27 (1H, dd, ArH), 7.99 |
| | (1H, d, ArH), 7.88 (1H, d, ArH), 7.59 (1H, t, ArH), 7.48-7.44 (2H, m, |
| | ArH), 7.37 (2H, t, ArH), 4.08 (2H, s, CH₂), 3.55 (2H, q, CH₂), 3.15 (4H, |
| | s, CH₂), 2.97 (4H, s, CH₂), 1.41 (3H, t, CH₃). |
| A-05 | ¹H NMR δ (ppm) (DMSO-d₆): 8.03 (1H, d, ArH), 7.91 (1H, d, ArH), 7.61 |
| | (1H, d, ArH), 7.51-7.44 (2H, m, ArH), 7.38 (1H, t, ArH), 7.27 (1H, d, |
| | ArH), 7.18 (1H, t, ArH), 3.96 (2H, s, NCH₂), 2.79 (4H, br. s, CH₂), 2.61 |
| | (4H, br. s, CH₂). |
| A-06 | ¹H NMR δ (ppm) (CHCl₃-d): 8.41 (1H, d, ArH), 7.99 (1H, d, ArH), 7.86 |
| | (1H, d, ArH), 7.52-7.42 (2H, m, ArH), 7.39-7.34 (3H, m, ArH), 3.18 (2H, ~d, |
| | CH₂), 3.11-3.06 (2H, m, CH₂), 2.90 (2H, ~t, CH₂), 2.36-2.18 (1H, m, |
| | CH), 2.07 (2H, ~d, CH₂), 1.68 (2H, ~q, CH₂). |
| A-07 | ¹H NMR δ (ppm) (MeOH-d4): 8.68 (1H, d, ArH), 8.23-8.20 (1H, m, |
| | ArH), 8.11 (1H, d, ArH), 7.63-7.54 (4H, m, ArH), 4.35 (2H, s, CH₂), 4.03 |
| | (4H, ~t, CH₂), 3.37 (4H, ~t, CH₂). |
| A-08 | ¹H NMR δ (ppm) (CHCl₃-d): 8.25 (1H, d, ArH), 7.75 (1H, d, ArH), 7.61- |
| | 7.58 (2H, m, ArH), 7.38-7.26 (3H, m, ArH), 4.08 (2H, q, OCH₂), 3.94 |
| | (2H, s, NCH₂), 3.91 (3H, s, NCH₃), 3.05-3.03 (4H, m, CH₂), 2.78 (4H, br. |
| | s, CH₂), 1.14 (3H, t, CH₃). |
| A-09 | ¹H NMR δ (ppm) (CHCl₃-d): 8.25 (1H, d, ArH), 7.75 (1H, d, ArH), 7.61- |
| | 7.58 (2H, m, ArH), 7.38-7.36 (1H, m, ArH), 7.32-7.25 (2H, m, ArH), 4.02 |
| | (2H, t, OCH₂), 3.95 (2H, s, NCH₂), 3.91 (3H, s, NCH₃), 3.05-3.03 (4H, m, |
| | CH₂), 2.79 (4H, br. s, CH₂), 1.47-1.41 (2H, m, CH₂), 1.24-1.15 (2H, m, |
| | CH₂), 0.82 (3H, t, CH₃). |
| A-10 | ¹H NMR δ (ppm) (DMSO-d₆): 10.0 (1H, s, NH), 8.07 (1H, d, ArH), 7.78 |
| | (1H, s, ArH), 7.60 (1H, d, ArH), 7.56-7.50 (2H, m, ArH), 7.23-7.12 (2H, |
| | m, ArH), 6.52-6.49 (1H, m, NH), 3.86 (5H, s, NCH₂, NCH₃), 2.94-2.85 |
| | (6H, m, CH₂), 2.65 (4H, br. s, CH₂), 1.29-1.19 (2H, m, CH₂), 1.15-1.09 |
| | (2H, m, CH₂), 0.77-0.73 (3H, ~t, CH₃). |
| A-11 | ¹H NMR δ (ppm) (DMSO-d₆): 11.2 (1H, br. s, NH), 7.76 (1H, d, ArH), |
| | 7.57-7.50 (2H, m, ArH), 7.30 (1H, s, ArH), 7.21-7.19 (1H, m, ArH), 7.17- |
| | 7.10 (2H, m, ArH), 3.89-3.86 (7H, m, NCH₂, NCH₃, OCH₂), 2.83 (4H, br. |
| | s, CH₂), 2.67 (4H, br. s, CH₂), 2.32 (3H, s, CH₃), 1.35-1.31 (2H, m, CH₂), |
| | 1.13-1.07 (2H, m, CH₂), 0.75 (3H, t, CH₃). |
| A-12 | ¹H NMR δ (ppm) (DMSO-d₆): 9.48 (1H, s, NH), 7.73 (1H, d, ArH), 7.57- |
| | 7.50 (2H, m, ArH), 7.31 (1H, s, ArH), 7.23-7.19 (1H, m, ArH), 7.17-7.10 |
| | (2H, m, ArH), 6.54 (1H, t, NH), 3.86 (5H, s, NCH₂, NCH₃), 2.88 (2H, q, |
| | NCH₂), 2.82 (4H, br. s, CH₂), 2.65 (4H, br. s, CH₂), 2.31 (3H, s, CH₃), |
| | 1.24-1.19 (2H, m, CH₂), 1.15-1.10 (2H, m CH₂), 0.75 (3H, t, CH₃). |
| A-13 | ¹H NMR δ (ppm) (DMSO-d₆): 14.67 (1H, s, NH), 7.91 (1H, d, ArH), |
| | 7.63-7.51 (4H, m, ArH), 7.33 (1H, t, ArH), 7.22 (1H, t, ArH), 7.16 (1H, t, |
| | ArH), 3.86 (3H, s, NCH₃), 3.83 (2H, s, NCH₂), 3.48 (2H, q, CH₂), 3.02 |
| | (4H, d, CH₂), 2.69 (4H, br. s, CH₂), 1.26 (3H, t, CH₃). |
| A-14 | ¹H NMR δ (ppm) (DMSO-d₆): 14.57 (1H, br. s, NH), 7.91 (1H, d, ArH), |
| | 7.58-7.51 (2H, m, ArH), 7.22 (1H, t, ArH), 7.16 (1H, t, ArH), 7.00 (1H, d, |
| | ArH), 6.92-6.89 (1H, m, ArH), 4.78-4.75 (1H, m, CH), 3.87 (3H, s, |
| | NCH₃), 3.82 (2H, s, NCH₂), 3.49-3.44 (2H, m, CH₂), 3.01 (4H, br. s, |
| | CH₂), 2.69 (4H, br. s, CH₂), 1.26-1.20 (9H, m, CH₃). |
| A-15 | ¹H NMR δ (ppm) (DMSO-d6): 7.60-7.53 (2H, m, ArH), 7.48 (2H, ~dd, |
| | ArH), 7.21-7.14 (4H, m, ArH), 3.80 (3H, s, CH₃), 3.77 (2H, s, CH₂), 3.31 |
| | (4H, br. s, CH₂), 2.71 (4H, s, CH₂). |
| A-16 | ¹H NMR δ (ppm) (DMSO-d6): 7.54 (1H, d, ArH), 7.49 (2H, ~d, ArH), |
| | 7.20 (1H, t, ArH), 7.14 (1H, t, ArH), 7.04 (1H, s, ArH), 6.98 (1H, d, |
| | ArH), 3.80 (3H, s, NCH₃), 3.78 (2H, s, NCH₂), 2.70 (4H, br. s, CH₂), 2.47 |
| | (4H, s, CH₂), 2.32 (3H, s, CH₃). |
| A-17 | ¹H NMR δ (ppm) (DMSO-d6): 7.90 (1H, d, ArH), 7.64 (1H, d, ArH), 7.59 |
| | (2H, ~d, ArH), 7.52 (1H, s, ArH), 7.33-7.22 (2H, m, ArH), 3.90 (5H, s, |
| | NCH₃, NCH₂), 2.88 (4H, br. s, CH₂), 2.57 (4H, s, CH₂). |
| A-18 | ¹H NMR δ (ppm) (DMSO-d6): 7.55 (2H, d, ArH), 7.50 (1H, d, ArH), |
| | 7.21-7.15 (5H, m, ArH), 6.43 (2H, d, CH), 3.82 (3H, s, NCH₃), 3.80 (2H, |
| | s, NCH₂), 2.74 (4H, s, CH₂), 2.48 (4H, br. s, CH₂), 1.85 (3H, ~d, CH₃). |
| A-19 | ¹H NMR δ (ppm) (MeOH-d4): 7.60 (2H, d, ArH), 7.51 (1H, d, ArH), |
| | 7.31-7.26 (2H, m, ArH), 7.11 (1H, s, ArH), 7.05 (1H, d, ArH), 3.92 (3H, |
| | s, NCH₃), 3.90 (2H, s, NCH₂), 2.85 (4H, ~t, CH₂), 2.63 (6H, br. s, CH₂), |
| | 1.69-1.63 (2H, m, CH₂), 0.95 (3H, t, CH₃). |
| A-20 | ¹H NMR δ (ppm) (CHCl₃-d): 8.28 (1H, d, ArH), 7.77 (1H, d, ArH), 7.39- |
| | 7.27 (3H, m, ArH), 7.16-7.12 (2H, m, ArH), 4.00 (2H, s, NCH₂), 3.92 |
| | (3H, s, NCH₃), 3.00 (4H, ~t, CH₂), 2.86 (4H, br. s, CH₂), 2.51 (2H, q, |
| | CH₂), 1.90-1.84 (1H, m, CH), 0.90 (6H, d, CH₃). |
| A-21 | ¹H NMR δ (ppm) (DMSO-d6): 7.58-7.54 (2H, m, ArH), 7.49 (1H, d, |
| | ArH), 7.22-7.18 (1H, m, ArH), 7.16-7.12 (1H, m, ArH), 7.03 (2H, d, |
| | ArH), 6.28 (1H, s, CH), 3.80 (3H, s, NCH₃), 3.78 (2H, s, NCH₂), 2.71 |
| | (4H, ~t, CH₂), 2.50 (4H, br. s, CH₂), 1.87 (3H, s, CH₃), 1.84 (3H, s, CH₃). |
| A-22 | ¹H NMR δ (ppm) (DMSO-d6): 7.56-7.53 (2H, m, ArH), 7.48 (1H, d, |
| | ArH), 7.20 (1H, t, ArH), 7.13 (1H, t, ArH), 6.75-6.72 (1H, m, ArH), 6.69 |
| | (1H, d, ArH), 3.80 (5H, s, NCH₃, NCH₂), 3.78 (3H, s, OCH₃), 2.70 (4H, |
| | br. s, CH₂), 2.47 (4H, s, CH₂). |
| A-23 | ¹H NMR δ (ppm) (DMSO-d6): 7.54 (2H, d, ArH), 7.48 (1H, d, ArH), |
| | 7.20-12 (2H, m, ArH), 6.73-6.71 (1H, m, ArH), 6.66 (1H, d, ArH), 4.05 |
| | (2H, q, OCH₂), 3.80 (3H, s, NCH₃), 3.77 (2H, s, NCH₂), 2.68 (4H, ~t, |
| | CH₂), 2.47 (4H, br. s, CH₂), 1.30 (3H, t, CH₃). |
| A-24 | ¹H NMR δ (ppm) (DMSO-d6): 7.55-7.47 (3H, m, ArH), 7.18 (1H, t, |
| | ArH), 7.14 (1H, t, ArH), 6.73-6.71 (1H, m, ArH), 6.63 (1H, d, ArH), 4.68 |
| | (1H, sept, CH), 3.80 (3H, s, NCH₃), 3.77 (2H, s, NCH₂), 2.69 (4H, s, |
| | CH₂), 2.47 (4H, br. s, CH₂), 1.24 (6H, d, CH₃). |
| A-25 | ¹H NMR δ (ppm) (DMSO-d6): 7.55 (1H, d, ArH), 7.51 (1H, d, ArH), 7.45 |
| | (1H, d, ArH), 7.17-7.09 (2H, m, ArH), 6.73-6.70 (1H, m, ArH), 6.65 (1H, |
| | d, ArH), 4.68 (1H, sept, OCH), 3.73 (3H, s, NCH₃), 2.87 (2H, ~d, NCH₂), |
| | 2.80 (2H, d, CH₂), 2.56-2.52 (2H, m, CH₂), 1.93-1.92 (1H, m, CH), 1.66- |
| | 1.63 (2H, m, CH₂), 1.50-1.44 (2H, m, CH₂), 1.26 (6H, d, CH₂). |
| A-26 | ¹H NMR δ (ppm) (MeOH-d4): 8.19 (1H, d, ArH), 7.83-7.79 (1H, m, |
| | ArH), 7.69 (2H, t, ArH), 7.55-7.50 (2H, m, ArH), 7.33 (1H, d, ArH), 7.22 |
| | (1H, t, ArH), 3.64 (2H, s, CH₂), 2.93 (4H, ~t, CH₂), 2.71 (4H, br. s, CH₂). |
| A-27 | ¹H NMR δ (ppm) (DMSO-d6): 8.09-8.06 (1H, m, ArH), 7.79-7.75 (1H, m, |
| | ArH), 7.61 (1H, d, ArH), 7.60-7.45 (2H, m, ArH), 7.06 (1H, s, ArH), 6.98 |
| | (1H, d, ArH), 5.72 (1H, s, NH), 3.45 (2H, s, NCH₂), 2.74 (4H, br. s, CH₂), |
| | 2.57 (4H, s, CH₂), 2.33 (3H, s, CH₃). |
| A-28 | ¹H NMR δ (ppm) (DMSO-d6): 11.91 (1H, br. s, NH), 8.07 (1H, d, ArH), |
| | 7.77 (1H, ~t, ArH), 7.61 (1H, d, ArH), 7.46 (2H, ~dd, ArH), 6.94 (1H, s, |
| | ArH), 6.81 (1H, d, ArH), 3.45 (2H, s, NCH₂), 2.74 (4H, ~t, CH₂), 2.56 |
| | (4H, br. s, CH₂), 1.97-1.92 (1H, m, CH), 1.00-0.94 (2H, m, CH₂), 0.75- |
| | 0.71 (2H, m, CH₂). |
| A-29 | ¹H NMR δ (ppm) (DMSO-d6): 8.08-8.06 (1H, m, ArH), 7.81-7.75 (2H, m, |
| | ArH), 7.62 (1H, d, ArH), 7.50-7.44 (3H, m, ArH), 3.48 (2H, s, NCH₂), |
| | 2.82 (4H, br. s, CH₂), 2.58 (4H, s, CH₂). |
| A-30 | ¹H NMR δ (ppm) (DMSO-d6): 8.08 (1H, d, ArH), 7.77 (1H, ~t, ArH), |
| | 7.64-7.57 (2H, m, ArH), 7.48 (1H, t, ArH), 7.04 (2H, d, ArH), 6.30 (1H, s, |
| | CH), 3.46 (2H, s, NCH₂), 2.76 (4H, s, CH₂), 2.58 (4H, s, CH₂), 1.88 (3H, |
| | s, CH₃), 1.86 (3H, s, CH₃). |
| A-31 | ¹H NMR δ (ppm) (DMSO-d6): 8.07 (1H, d, ArH), 7.78 (1H, ~t, ArH), |
| | 7.63 (1H, d, ArH), 7.53-7.46 (2H, m, ArH), 7.02 (1H, s, ArH), 6.97 (1H, |
| | d, ArH), 3.46 (2H, s, NCH₂), 2.75 (4H, s, CH₂), 2.57 (4H, s, CH₂), 2.48 |
| | (2H, br. s, CH₂), 1.89-1.82 (1H, m, CH), 0.86 (6H, d, CH₃). |
| A-32 | ¹H NMR δ (ppm) (DMSO-d6): 8.07 (1H, d, ArH), 7.79-7.75 (1H, m, |
| | ArH), 7.62 (1H, d, ArH), 7.53 (1H, d, ArH), 7.47 (1H, t, ArH), 6.74-6.71 |
| | (1H, m, ArH), 6.65 (1H, d, ArH), 4.72-4.66 (1H, m, OCH), 3.44 (2H, s, |
| | NCH₂), 2.72 (4H, s, CH₂), 2.56 (4H, s, CH₂), 1.25 (6H, d, CH₃). |
| A-33 | ¹H NMR δ (ppm) (CHCl₃-d): 14.79 (1H, s, NH), 8.16 (1H, d, ArH), 7.99 |
| | (1H, d, ArH), 7.87 (1H, d, ArH), 7.48-7.37 (3H, m, ArH), 7.22-7.18 (1H, |
| | m, ArH), 4.07 (2H, s, NCH₂), 3.53 (2H, q, CH₂), 3.13 (4H, ~t, CH₂), 2.96 |
| | (4H, br. s, CH₂), 2.71 (2H, q, CH₂), 1.39 (3H, t, CH₃), 1.26 (3H, t, CH₃). |
| A-34 | ¹H NMR δ (ppm) (DMSO-d₆): 14.70 (1H, s, NH), 8.05 (1H, d, ArH), |
| | 7.93-7.86 (2H, m, ArH), 7.47-7.39 (3H, m, ArH), 7.24 (1H, d, ArH), 4.01 |
| | (2H, s, NCH₂), 3.44 (2H, q, CH₂), 3.09 (4H, d, CH₂), 2.95 (1H, br. s, CH), |
| | 2.80 (4H, br. s, CH₂), 1.20 (9H, br. s, CH₃). |
| A-35 | ¹H NMR δ (ppm) (DMSO-d₆): 14.76 (1H, s, NH), 8.06 (1H, d, ArH), 7.93 |
| | (1H, d, ArH), 7.83 (1H, d, ArH), 7.46 (1H, t, ArH), 7.40 (1H, t, ArH), |
| | 7.33 (1H, s, ArH), 7.02 (1H, d, ArH), 4.02 (2H, s, NCH₂), 3.45 (2H, q, |
| | CH₂), 3.08 (4H, ~t, CH₂), 2.81 (4H, br. s, CH₂), 2.03-1.99 (1H, m, CH), |
| | 1.23 (3H, t, CH₃), 1.06-1.01 (2H, m, CH₂), 0.83-0.79 (2H, m, CH₂). |
| A-36 | ¹H NMR δ (ppm) (DMSO-d₆): 14.74 (1H, br. s, NH), 8.05 (1H, d, ArH), |
| | 7.92 (1H, d, ArH), 7.86 (1H, d, ArH), 7.48-7.38 (3H, m, ArH), 7.15 (1H, |
| | d, ArH), 4.01 (2H, s, NCH₂), 3.45 (2H, q, CH₂), 3.08 (4H, s, CH₂), 2.80 |
| | (4H, br. s, CH₂), 2.51 (2H, d, CH₂), 1.89-1.84 (1H, m, CH), 1.23 (3H, t, |
| | CH₃), 0.84 (6H, d, CH₃). |
| A-37 | ¹H NMR δ (ppm) (DMSO-d₆): 14.38 (1H, br. s, NH), 8.05 (1H, d, ArH), |
| | 7.92 (2H, t, ArH), 7.47 (1H, t, ArH), 7.39 (1H, t, ArH), 7.08 (1H, s, ArH), |
| | 6.91 (1H, d, ArH), 4.01 (2H, s, NCH₂), 3.84 (3H, s, OCH₃), 3.44 (2H, q, |
| | CH₂), 3.07 (4H, s, CH₂), 2.80 (4H, br. s, CH₂), 1.22 (3H, t, CH₃). |
| A-38 | ¹H NMR δ (ppm) (DMSO-d₆): 14.41 (1H, s, NH), 8.06 (1H, d, ArH), 7.92 |
| | (2H, t, ArH), 7.47 (1H, t, ArH), 7.40 (1H, t, ArH), 7.07 (1H, d, ArH), |
| | 6.93-6.90 (1H, m, ArH), 4.13 (2H, q, OCH₂), 4.02 (2H, s, NCH₂), 3.46 |
| | (2H, q, CH₂), 3.07 (4H, d, CH₂), 2.80 (4H, br. s, CH₂), 1.33 (3H, t, CH₃), |
| | 1.23 (3H, t, CH₃). |
| A-39 | ¹H NMR δ (ppm) (DMSO-d₆): 15.62 (1H, br. s, NH), 7.84 (1H, d, ArH), |
| | 7.77 (1H, d, ArH), 7.47 (1H, d, ArH), 7.29-7.25 (1H, m, ArH), 7.10-7.02 |
| | (2H, m, ArH), 6.93 (1H, d, ArH), 4.70 (1H, ~sept, CH), 3.91 (2H, s, |
| | NCH₂), 3.67 (4H, s, CH₂), 3.40 (2H, q, CH₂), 2.78 (4H, s, CH₂), 1.28-1.21 |
| | (9H, m, CH₃). |
| A-40 | ¹H NMR δ (ppm) (DMSO-d₆): 14.16 (1H, br. s, NH), 8.05 (1H, d, ArH), |
| | 7.94-7.89 (2H, m, ArH), 7.48 (1H, t, ArH), 7.40 (1H, t, ArH), 7.04 (1H, d, |
| | ArH), 6.92-6.89 (1H, m, ArH), 4.81-4.79 (1H, m, CH), 4.02 (2H, s, |
| | NCH₂), 3.47 (2H, q, CH₂), 3.07 (4H, ~t, CH₂), 2.81 (4H, s, CH₂), 1.28- |
| | 1.22 (9H, m, CH₃). |
| A-41 | ¹H NMR δ (ppm) (CHCl₃-d): 14.32 (1H, s, NH), 8.20 (1H, d, ArH), 7.99 |
| | (1H, d, ArH), 7.87 (1H, d, ArH), 7.46 (1H, t, ArH), 7.37 (1H, t, ArH), |
| | 7.06 (1H, d, ArH), 7.00 (1H, s, ArH), 4.07 (2H, s, NCH₂), 3.79 (1H, s, |
| | OCH), 3.54 (2H, q, CH₂), 3.11 (4H, s, CH₂), 2.95 (4H, br. s, CH₂), 1.40 |
| | (3H, t, CH₃), 0.88-0.79 (4H, m, CH₂). |
| A-42 | ¹H NMR δ (ppm) (CHCl₃-d): 14.36 (1H, s, NH), 8.19 (1H, d, ArH), 7.99 |
| | (1H, d, ArH), 7.88 (1H, d, ArH), 7.47 (1H, t, ArH), 7.39 (1H, t, ArH), |
| | 6.90 (1H, d, ArH), 6.86-6.83 (1H, m, ArH), 4.08 (2H, s, NCH₂), 3.98 (2H, |
| | t, OCH₂), 3.54 (2H, q, CH₂), 3.14 (4H, ~t, CH₂), 2.96 (4H, br. s, CH₂), |
| | 1.86-1.81 (2H, m, CH₂), 1.40 (3H, t, CH₃), 1.06 (3H, t, CH₃). |
| A-43 | ¹H NMR δ (ppm) (DMSO-d₆): 14.46 (1H, s, NH), 8.06 (1H, d, ArH), 7.92 |
| | (2H, t, ArH), 7.48 (1H, t, ArH), 7.40 (1H, t, ArH), 7.10 (1H, d, ArH), |
| | 6.94-6.91 (1H, m, ArH), 4.02 (2H, s, NCH₂), 3.85 (2H, d, OCH₂), 3.46 |
| | (2H, q, CH₂), 3.08 (4H, d, CH₂), 2.81 (4H, br. s, CH₂), 2.05-1.98 (1H, m, |
| | CH), 1.23 (3H, t, CH₃), 0.97 (6H, d, CH₃). |
| A-44 | ¹H NMR δ (ppm) (DMSO-d₆): 14.52 (1H, s, NH), 8.04 (1H, d, ArH), 7.92 |
| | (1H, d, ArH), 7.74 (1H, d, ArH), 7.45 (1H, t, ArH), 7.40 (1H, t, ArH), |
| | 6.66-6.53 (2H, m, ArH), 6.49 (1H, d, NH), 4.00 (2H, s, NCH₂), 3.41 (2H, |
| | q, CH₂), 3.11 (2H, q, CH₂), 3.00 (4H, ~t, CH₂), 2.78 (4H, br. s, CH₂), 1.20 |
| | (3H, d, CH₃), 1.14 (3H, d, CH₃). |
| A-45 | ¹H NMR δ (ppm) (MeOH-d4): 7.95 (1H, d, ArH), 7.90 (1H, d, ArH), 7.63 |
| | (1H, d, ArH), 7.48 (1H, t, ArH), 7.40 (1H, t, ArH), 7.18 (1H, s, ArH), |
| | 7.09 (1H, d, ArH), 4.03 (2H, s, NCH₂), 2.92 (4H, ~t, CH₂), 2.75-2.68 (6H, |
| | m, CH₂), 1.26 (3H, t, CH₃). |
| A-46 | ¹H NMR δ (ppm) (MeOH-d4): 7.95 (1H, d, ArH), 7.90 (1H, d, ArH), 7.65 |
| | (1H, d, ArH), 7.48 (1H, t, ArH), 7.40 (1H, t, ArH), 7.20 (1H, s, ArH), |
| | 7.13 (1H, d, ArH), 4.04 (2H, s, NCH₂), 2.99-2.93 (5H, m, CH, CH₂), 2.76 |
| | (4H, br. s, CH₂), 1.29 (6H, d, CH₃). |
| A-47 | ¹H NMR δ (ppm) (MeOH-d4): 7.96 (1H, d, ArH), 7.91 (1H, d, ArH), 7.60 |
| | (1H, d, ArH), 7.48 (1H, t, ArH), 7.40 (1H, t, ArH), 7.07 (1H, s, ArH), |
| | 6.90 (1H, d, ArH), 4.04 (2H, s, NCH₂), 2.92 (4H, ~t, CH₂), 2.76 (4H, br. s, |
| | CH₂), 2.01-1.96 (1H, m, CH), 1.07-1.03 (2H, m, CH₂), 0.80-0.76 (2H, m, |
| | CH₂). |
| A-48 | ¹H NMR δ (ppm) (MeOH-d4): 7.95 (1H, d, ArH), 7.90 (1H, d, ArH), 7.63 |
| | (1H, d, ArH), 7.48 (1H, ~td, ArH), 7.40 (1H, ~td, ArH), 7.13 (1H, s, |
| | ArH), 7.04 (1H, ~dd, ArH), 4.04 (2H, s, NCH₂), 2.92 (4H, ~t, CH₂), 2.76 |
| | (4H, br. s, CH₂), 2.55 (2H, d, CH₂), 1.94-1.90 (1H, m, CH), 0.93 (6H, d, |
| | CH₃). |
| A-49 | ¹H NMR δ (ppm) (MeOH-d4): 7.66 (2H, t, ArH), 7.47 (1H, d, ArH), 7.31- |
| | 7.26 (1H, m, ArH), 7.15 (1H, s, ArH), 7.11-7.07 (2H, m, ArH), 3.69 (4H, ~t, |
| | CH₂), 2.98 (4H, ~t, CH₂), 2.56 (2H, d, CH₂), 1.94-1.91 (1H, m, CH), |
| | 0.93 (6H, d, CH₃). |
| A-50 | ¹H NMR δ (ppm) (MeOH-d4): 8.01 (1H, d, ArH), 7.70-7.68 (1H, m, |
| | ArH), 7.52 (1H, d, ArH), 7.34-7.30 (1H, m, ArH), 7.16-7.11 (3H, m, |
| | ArH), 4.72 (1H, ~sept, CH), 4.28 (2H, s, NCH₂), 3.92 (4H, ~t, CH₂), 3.35 |
| | (4H, ~t, CH₂), 1.35 (6H, d, CH₃). |
| A-51 | ¹H NMR δ (ppm) (DMSO-d6): 8.03 (1H, d, ArH), 7.90 (1H, d, ArH), 7.54 |
| | (1H, d, ArH), 7.45 (1H, t, ArH), 7.38 (1H, t, ArH), 6.75-6.73 (1H, m, |
| | ArH), 6.69 (1H, d, ArH), 4.74-4.68 (1H, m, CH), 3.96 (2H, s, NCH₂), |
| | 2.75 (4H, ~t, CH₂), 2.62 (4H, s, CH₂), 1.27 (6H, d, CH₃). |
| A-52 | ¹H NMR δ (ppm) (DMSO-d6): 8.02 (1H, d, ArH), 7.89 (1H, d, ArH), 7.57 |
| | (1H, d, ArH), 7.43 (1H, t, ArH), 7.37 (1H, t, ArH), 6.89 (1H, d, ArH), |
| | 6.83 (1H, s, ArH), 3.95 (2H, s, NCH₂), 3.95-3.91 (1H, m, CH), 2.76 (4H, ~t, |
| | CH₂), 2.62 (4H, br. s, CH₂), 0.79 (2H, br. s, CH₂), 0.66 (2H, br. s, |
| | CH₂). |
| A-53 | ¹H NMR δ (ppm) (MeOH-d4): 7.95 (1H, d, ArH), 7.90 (1H, d, ArH), 7.64 |
| | (1H, d, ArH), 7.48 (1H, t, ArH), 7.40 (1H, t, ArH), 6.82 (1H, s, ArH), |
| | 6.78 (1H, ~d, ArH), 4.03 (2H, s, NCH₂), 3.82 (2H, d, OCH₂), 2.90 (4H, ~t, |
| | CH₂), 2.75 (4H, br. s, CH₂), 2.10-2.05 (1H, m, CH), 1.05 (6H, d, CH₃). |
| A-54 | ¹H NMR δ (ppm) (MeOH-d4): 7.95 (1H, d, ArH), 7.90 (1H, d, ArH), 7.64 |
| | (1H, d, ArH), 7.48 (1H, t, ArH), 7.40 (1H, t, ArH), 6.82 (1H, s, ArH), |
| | 6.76 (1H, ~d, ArH), 4.03 (2H, s, NCH₂), 3.89 (2H, d, OCH₂), 2.90 (4H, ~t, |
| | CH₂), 2.75 (4H, br. s, CH₂), 1.29-1.25 (1H, m, CH), 0.65-0.61 (2H, m, |
| | CH₂), 0.39-0.35 (2H, m, CH₂). |
| A-55 | ¹H NMR δ (ppm) (MeOH-d4): 7.94 (1H, d, ArH), 7.90 (1H, d, ArH), 7.66 |
| | (1H, d, ArH), 7.47 (1H, t, ArH), 7.39 (1H, t, ArH), 6.88 (1H, s, ArH), |
| | 6.83 (1H, ~d, ArH), 6.19 (1H, ~tt, CH), 4.31 (2H, td, OCH₂), 4.03 (2H, s, |
| | NCH₂), 2.89 (4H, ~t, CH₂), 2.74 (4H, br. s, CH₂). |
| A-56 | ¹H NMR δ (ppm) (MeOH-d4): 7.95 (1H, d, ArH), 7.90 (1H, d, ArH), 7.65 |
| | (1H, d, ArH), 7.48 (1H, t, ArH), 7.40 (1H, t, ArH), 6.86 (1H, s, ArH), |
| | 6.80 (1H, ~d, ArH), 4.19 (2H, t, OCH₂), 4.05 (2H, s, NCH₂), 3.76 (2H, t, |
| | OCH₂), 3.42 (3H, s, OCH₃), 2.91 (4H, ~t, CH₂), 2.77 (4H, br. s, CH₂). |
| A-57 | ¹H NMR δ (ppm) (MeOH-d4): 7.95 (1H, d, ArH), 7.90 (1H, d, ArH), 7.63 |
| | (1H, d, ArH), 7.48 (1H, t, ArH), 7.40 (1H, t, ArH), 6.78-6.74 (2H, m, |
| | ArH), 4.03 (2H, s, NCH₂), 3.31-3.28 (1H, m, CH), 2.89 (4H, ~t, CH₂), |
| | 2.75 (4H, br. s, CH₂), 2.01-1.95 (2H, m, CH₂), 1.84-1.77 (4H, m, CH₂), |
| | 1.68-1.64 (2H, m, CH₂). |
| A-58 | ¹H NMR δ (ppm) (MeOH-d4): 7.95 (1H, d, ArH), 7.90 (1H, d, ArH), 7.66 |
| | (1H, d, ArH), 7.46 (1H, t, ArH), 7.40 (1H, t, ArH), 6.88 (1H, s, ArH), |
| | 6.84 (1H, d, ArH), 4.90 (2H, br. s, OCH₂), 4.61 (2H, t, OCH₂), 4.28 (2H, |
| | d, OCH₂), 4.03 (2H, s, NCH₂), 3.49-3.42 (1H, m, CH), 2.90 (4H, ~t, CH₂), |
| | 2.75 (4H, br. s, CH₂). |
| A-59 | ¹H NMR δ (ppm) (MeOH-d4): 7.95 (1H, d, ArH), 7.90 (1H, d, ArH), 7.54 |
| | (1H, d, ArH), 7.48 (1H, t, ArH), 7.42 (1H, t, ArH), 6.50 (1H, ~d, ArH), |
| | 6.41 (1H, ~d, ArH), 4.03 (2H, s, NCH₂), 3.17 (2H, q, NCH₂), 2.90 |
| | (4H, ~t, CH₂), 2.78 (4H, br. s, CH₂), 1.25 (3H, t, CH₃). |
| A-60 | ¹H NMR δ (ppm) (MeOH-d4): 8.00 (2H, ~t, ArH), 7.56-7.44 (3H, m, |
| | ArH), 6.50 (1H, ~d, ArH), 6.46 (1H, d, ArH), 4.47 (2H, br. s, NCH₂), 3.68 |
| | (1H, ~sept, NCH₂), 3.21 (4H, br. s, CH₂), 3.06 (4H, br. s, CH₂), 1.23 (6H, |
| | d, CH₃). |
| A-61 | ¹H NMR δ (ppm) (DMSO-d6): 8.20 (1H, d, ArH), 8.09 (1H, d, ArH), |
| | 7.60-7.52 (3H, m, ArH), 6.57 (1H, d, ArH), 6.52 (1H, s, ArH), 4.91 (2H, |
| | s, NCH₂), 3.53 (4H, br. s, CH₂), 3.07 (4H, br. s, CH₂), 2.37 (2H, ~t, CH), |
| | 0.70 (2H, ~d, CH₂), 0.38 (2H, s, CH₂). |
| A-62 | ¹H NMR δ (ppm) (MeOH-d4): 7.96 (1H, d, ArH), 7.91 (1H, d, ArH), 7.54 |
| | (1H, d, ArH), 7.48 (1H, t, ArH), 7.40 (1H, t, ArH), 6.53 (1H, ~d, ArH), |
| | 6.44 (1H, ~d, ArH), 4.07 (2H, s, NCH₂), 3.01 (2H, d, NCH₂), 2.92 (4H, ~t, |
| | CH₂), 2.82 (4H, br. s, CH₂), 1.11-1.08 (1H, m, CH), 0.57-0.52 (2H, m, |
| | CH₂), 0.28-0.24 (2H, m, CH₂). |
| A-63 | ¹H NMR δ (ppm) (MeOH-d4): 7.96 (1H, d, ArH), 7.90 (1H, d, ArH), 7.55 |
| | (1H, d, ArH), 7.48 (1H, t, ArH), 7.40 (1H, t, ArH), 6.56 (1H, ~d, ArH), |
| | 6.46 (1H, ~d, ArH), 4.04 (2H, s, NCH₂), 3.59 (2H, t, NCH₂), 3.38 (3H, s, |
| | OCH₃), 3.34 (2H, t, OCH₂), 2.91 (4H, ~t, CH₂), 2.78 (4H, br. s, CH₂) |
| A-64 | ¹H NMR δ (ppm) (MeOH-d4): 7.96 (1H, d, ArH), 7.90 (1H, d, ArH), 7.54 |
| | (1H, d, ArH), 7.48 (1H, t, ArH), 7.40 (1H, t, ArH), 6.51 (1H, ~d, ArH), |
| | 6.43 (1H, d, ArH), 4.04 (2H, s, NCH₂), 3.83 (1H, ~quint, CH), 2.90 (4H, ~t, |
| | CH₂), 2.78 (4H, br. s, CH₂), 2.04-1.98 (2H, m, CH₂), 1.78-1.71 (2H, m, |
| | CH₂), 1.69-1.63 (2H, m, CH₂), 1.57-1.51 (2H, m, CH₂). |
| A-65 | ¹H NMR δ (ppm) (MeOH-d4): 7.95 (1H, d, ArH), 7.90 (1H, d, ArH), 7.61 |
| | (1H, d, ArH), 7.48 (1H, t, ArH), 7.40 (1H, t, ArH), 6.78-6.75 (2H, m, |
| | ArH), 4.04 (2H, s, NCH₂), 3.33 (4H, t, CH₂), 2.93 (4H, ~t, CH₂), 2.78 |
| | (4H, br. s, CH₂), 1.68 (6H, br. s, CH₂). |
| A-66 | ¹H NMR δ (ppm) (MeOH-d4): 7.94 (1H, d, ArH), 7.89 (1H, d, ArH), 7.50 |
| | (1H, d, ArH), 7.47 (1H, t, ArH), 7.39 (1H, t, ArH), 6.74-6.72 (2H, m, |
| | ArH), 3.99 (2H, s, NCH₂), 3.38 (4H, ~t, CH₂), 2.95 (4H, ~t, CH₂), 2.90 |
| | (4H, ~t, CH₂), 2.70 (4H, br. s, CH₂), 2.62 (3H, s, CH₃). |
| A-67 | ¹H NMR δ (ppm) (MeOH-d4): 7.95 (1H, d, ArH), 7.90 (1H, d, ArH), 7.63 |
| | (1H, d, ArH), 7.48 (1H, t, ArH), 7.40 (1H, t, ArH), 6.80-6.77 (2H, m, |
| | ArH), 4.04 (2H, s, NCH₂), 3.83 (4H, t, CH₂), 3.30-3.26 (4H, m, CH₂), |
| | 2.93 (4H, ~t, CH₂), 2.77 (4H, br. s, CH₂), |
| A-68 | ¹H NMR δ (ppm) (MeOH-d4): 7.95 (1H, d, ArH), 7.90 (1H, d, ArH), 7.53 |
| | (1H, d, ArH), 7.48 (1H, t, ArH), 7.40 (1H, t, ArH), 6.46-6.32 (2H, m, |
| | ArH), 4.10-3.99 (1H, m, CH₂), 4.03 (2H, s, NCH₂), 3.76-3.55 (1H, m, |
| | CH₂), 3.25 (3H, s, CH₃), 2.90 (4H, ~t, CH₂), 2.88-2.81 (1H, m, CH₂), 2.77 |
| | (4H, br. s, CH₂), 2.42-2.17 (2H, m, CH₂), 1.79-1.76 (1H, m, CH₂). |
| A-69 | ¹H NMR δ (ppm) (MeOH-d4): 8.00 (1H, s, ArH), 7.63 (1H, d, ArH), 7.13 |
| | (1H, s, ArH), 7.06-7.04 (1H, m, ArH), 3.96 (3H, s, CH₃), 3.65 (2H, s, |
| | CH₂), 2.93 (4H, ~t, CH₂), 2.71 (4H, br. s, CH₂), 2.55 (2H, d, CH₂), 1.93- |
| | 1.90 (1H, m, CH), 0.93 (6H, d, CH₃). |
| A-70 | ¹H NMR δ (ppm) (MeOH-d4): 8.86 (1H, s, ArH), 7.89-7.86 (1H, m, |
| | ArH), 7.65 (1H, d, ArH), 7.59 (1H, d, ArH), 7.08 (1H, s, ArH), 7.03-7.01 |
| | (1H, m, ArH), 6.57 (1H, s, ArH), 3.86 (2H, s, CH₂), 3.00 (4H, ~t, CH₂), |
| | 2.91 (4H, br. s, CH₂), 2.54 (2H, d, CH₂), 2.45 (3H, s, CH₃), 1.94-1.88 (1H, |
| | m, CH), 0.93 (6H, d, CH₃). |
| A-71 | ¹H NMR δ (ppm) (MeOH-d4): 7.84-7.83 (1H, m, ArH), 7.62 (1H, d, |
| | ArH), 7.27 (1H, d, ArH), 7.28-7.26 (2H, m, ArH), 7.14-7.09 (1H, m, |
| | ArH), 3.37 (2H, br. s, CH₂), 2.98 (4H, ~t, CH₂), 2.81 (4H, br. s, CH₂), |
| | 2.56 (2H, d, CH₂), 1.94-1.91 (1H, m, CH), 0.94 (6H, d, CH₃). |
| A-72 | ¹H NMR δ (ppm) (MeOH-d4): 8.81 (1H, d, ArH), 7.51 (1H, d, ArH), 7.45 |
| | (1H, s, ArH), 7.18-7.16 (1H, m, ArH), 7.05 (1H, s, ArH), 6.96 (1H, d, |
| | ArH), 6.36 (1H, s, ArH), 3.48 (2H, s, CH₂), 2.76 (4H, br. s, CH₂), 2.53 |
| | (4H, br. s, CH₂), 2.42 (5H, br. s, CH₃, CH₂), 1.88-1.84 (1H, m, CH), 0.86 |
| | (6H, d, CH₃). |
| A-73 | ¹H NMR δ (ppm) (MeOH-d4): 7.65 (1H, ~d, ArH), 7.59 (1H, d, ArH), |
| | 7.44 (1H, d, ArH), 7.08 (1H, s, ArH), 7.03 (1H, d, ArH), 6.91 (1H, d, |
| | ArH), 6.40 (1H, s, ArH), 3.80 (2H, s, CH₂), 3.00 (7H, ~t, CH₂), 2.92 (4H, |
| | br. s, CH₂), 2.54 (2H, d, CH₂), 1.93-1.90 (1H, m, CH), 0.93 (6H, d, CH₃). |
| A-74 | ¹H NMR δ (ppm) (MeOH-d4): 8.05 (1H, d, ArH), 7.58 (1H, d, ArH), 7.05 |
| | (1H, s, ArH), 7.01 (1H, d, ArH), 6.39 (1H, d, ArH), 6.11 (1H, s, ArH), |
| | 4.34 (3H, s, CH₃), 3.80 (2H, s, CH₂), 2.99 (4H, ~d, CH₂), 2.93 (4H, br. s, |
| | CH₂), 2.53 (2H, d, CH₂), 1.93-1.89 (1H, m, CH), 0.92 (6H, d, CH₃). |
| A-75 | ¹H NMR δ (ppm) (MeOH-d4): 7.60 (1H, d, ArH), 7.08 (1H, s, ArH), 7.03 |
| | (1H, d, ArH), 6.88 (1H, d, ArH), 6.30 (1H, s, ArH), 3.72 (2H, s, CH₂), |
| | 2.97 (4H, ~t, CH₂), 2.86 (4H, br. s, CH₂), 2.76 (3H, s, CH₃), 2.54 (2H, d, |
| | CH₂), 1.93-1.89 (1H, m, CH), 0.93 (6H, d, CH₃). |
| A-76 | ¹H NMR δ (ppm) (MeOH-d4): 8.96 (1H, d, ArH), 7.91-7.86 (1H, m, |
| | ArH), 7.63 (1H, d, ArH), 7.51 (1H, d, ArH), 7.29-7.26 (1H, m, ArH), 7.00 |
| | (1H, s, ArH), 6.94 (1H, d, ArH), 6.52 (1H, s, ArH), 3.76 (2H, s, CH₂), |
| | 2.91 (4H, ~t, CH₂), 2.80 (4H, br. s, CH₂), 2.46 (2H, d, CH₂), 1.88-1.78 |
| | (1H, m, CH), 0.84 (6H, d, CH₃). |
| A-77 | ¹H NMR δ (ppm) (MeOH-d4): 8.10 (1H, s, ArH), 7.75-7.68 (2H, m, |
| | ArH), 7.60 (1H, d, ArH), 7.53 (1H, d, ArH), 7.35 (1H, t, ArH), 6.97 (2H, ~t, |
| | ArH), 4.13 (2H, s, CH₂), 3.77 (3H, s, CH₃), 3.16 (4H, br. s, CH₂), 3.04 |
| | (4H, br. s, CH₂), 2.51 (2H, d, CH₂), 1.91-1.88 (1H, m, CH), 0.91 (6H, d, |
| | CH₃). |
| A-78 | ¹H NMR δ (ppm) (MeOH-d4): 8.19 (1H, d, ArH), 7.83-7.78 (1H, m, |
| | ArH), 7.69 (1H, d, ArH), 7.61 (1H, d, ArH), 7.53 (1H, t, ArH), 7.09 (1H, |
| | s, ArH), 7.04-7.02 (1H, m, ArH), 3.86 (2H, s, CH₂), 3.75 (3H, s, CH₃), |
| | 2.91 (4H, ~t, CH₂), 2.79 (4H, br. s, CH₂), 2.53 (2H, d, CH₂), 1.91-1.88 |
| | (1H, m, CH), 0.91 (6H, d, CH₃). |
| A-79 | ¹H NMR δ (ppm) (MeOH-d4): 7.91 (1H, d, ArH), 7.68 (1H, t, ArH), 7.58 |
| | (2H, t, ArH), 7.43 (1H, t, ArH), 7.05 (1H, s, ArH), 7.01 (1H, d, ArH), |
| | 4.33 (2H, s, CH₂), 3.74 (3H, s, CH₃), 3.29 (4H, br. s, CH₂), 3.09 (4H, ~t, |
| | CH₂), 2.54 (2H, d, CH₂), 1.95-1.88 (1H, m, CH), 0.93 (6H, d, CH₃). |
| A-80 | ¹H NMR δ (ppm) (MeOH-d4): 8.57 (1H, d, ArH), 7.60-7.55 (3H, m, |
| | ArH), 7.45 (1H, ~t, ArH), 7.08-6.98 (3H, m, ArH), 4.00 (2H, s, CH₂), |
| | 2.85 (4H, br. s, CH₂), 2.63 (4H, br. s, CH₂), 2.51 (2H, d, CH₂), 1.90-1.86 |
| | (1H, m, CH), 0.90 (6H, d, CH₃). |
| A-81 | ¹H NMR δ (ppm) (MeOH-d4): 7.86 (1H, d, ArH), 7.57-7.43 (3H, m, |
| | ArH), 7.22 (1H, t, ArH), 6.95 (2H, s, ArH), 4.37 (2H, s, CH₂), 4.08 (3H, s, |
| | CH₃), 3.07 (4H, br. s, CH₂), 2.97 (4H, br. s, CH₂), 2.49 (2H, d, CH₂), |
| | 1.89-1.86 (1H, m, CH), 0.90 (6H, d, CH₃). |
| A-82 | ¹H NMR δ (ppm) (MeOH-d4): 7.61 (1H, d, ArH), 7.08 (1H, s, ArH), 7.03 |
| | (1H, d, ArH), 6.17 (1H, s, ArH), 3.71 (2H, s, CH₂), 2.89 (4H, ~t, CH₂), |
| | 2.69 (4H, br. s, CH₂), 2.54 (2H, d, CH₂), 2.41 (3H, s, CH₃), 1.92-1.89 (1H, |
| | m, CH), 0.92 (6H, d, CH₃). |
| A-83 | ¹H NMR δ (ppm) (MeOH-d4): 7.52 (1H, d, ArH), 7.19 (1H, d, ArH), |
| | 7.04-6.96 (3H, m, ArH), 3.78 (3H, s, CH₃), 3.72 (2H, s, CH₂), 2.84 |
| | (4H, ~t, CH₂), 2.55-2.51 (6H, m, CH₂), 1.91-1.88 (1H, m, CH), 0.91 (6H, d, |
| | CH₃). |
| A-84 | ¹H NMR δ (ppm) (MeOH-d4): 7.62 (1H, d, ArH), 7.36 (1H, d, ArH), 7.09 |
| | (1H, s, ArH), 7.04 (1H, d, ArH), 6.23 (1H, d, ArH), 3.87 (3H, s, CH₃), |
| | 3.70 (2H, s, CH₂), 2.89 (4H, t, CH₂), 2.63 (4H, br. s, CH₂), 2.54 (2H, d, |
| | CH₂), 1.92-1.89 (1H, m, CH), 0.92 (6H, d, CH₃). |
| A-85 | ¹H NMR δ (ppm) (MeOH-d4): 8.89 (1H, s, ArH), 8.18-8.15 (1H, m, |
| | ArH), 7.72 (1H, d, ArH), 7.62 (1H, d, ArH), 7.10 (1H, s, ArH), 7.05 (1H, |
| | d, ArH), 4.19 (2H, s, CH₂), 3.02 (8H, br. s, CH₂), 2.55 (2H, d, CH₂), 1.95- |
| | 1.88 (1H, m, CH), 0.93 (6H, d, CH₃). |
| A-86 | ¹H NMR δ (ppm) (MeOH-d4): 7.62 (1H, d, ArH), 7.10 (1H, s, ArH), 7.05- |
| | 7.03 (1H, m, ArH), 3.74 (2H, s, CH₂), 2.89 (4H, ~t, CH₂), 2.71 (4H, br. s, |
| | CH₂), 2.58 (3H, s, CH₃), 2.54 (2H, d, CH₂), 1.93-1.89 (1H, m, CH), 0.92 |
| | (6H, d, CH₃). |
| A-87 | ¹H NMR δ (ppm) (MeOH-d4): 7.54 (1H, d, ArH), 7.44 (1H, s, ArH), 7.00 |
| | (2H, ~t, ArH), 4.07 (2H, s, CH₂), 2.99 (8H, br. s, CH₂), 2.70 (3H, s, CH₃), |
| | 2.52 (2H, d, CH₂), 1.92-1.88 (1H, m, CH), 0.92 (6H, d, CH₃). |
| A-88 | ¹H NMR δ (ppm) (MeOH-d4): 8.81 (1H, d, ArH), 7.57 (1H, d, ArH), 7.44 |
| | (1H, t, ArH), 7.04 (1H, s, ArH), 7.01 (2H, d, ArH), 4.20 (2H, s, CH₂), |
| | 3.03 (8H, br. s, CH₂), 2.53 (2H, d, CH₂), 1.93-1.89 (1H, m, CH), 0.93 |
| | (6H, d, CH₃). |
| A-89 | ¹H NMR δ (ppm) (MeOH-d4): 7.61 (1H, d, ArH), 7.10 (1H, s, ArH), 7.05 |
| | (1H, d, ArH), 6.29 (1H, s, ArH), 3.85 (2H, s, CH₂), 2.91 (4H, t, CH₂), |
| | 2.72 (4H, br. s, CH₂), 2.54 (2H, d, CH₂), 2.27 (3H, s, CH₃), 1.93-1.89 (1H, |
| | m, CH), 0.92 (6H, d, CH₃). |
| A-90 | ¹H NMR δ (ppm) (MeOH-d4): 9.12-9.10 (1H, m, ArH), 7.87-7.84 (1H, m, |
| | ArH), 7.74-7.71 (1H, m, ArH), 7.61 (1H, d, ArH), 7.09 (1H, s, ArH), 7.04 |
| | (1H, d, ArH), 4.00 (2H, s, CH₂), 2.92 (4H, ~t, CH₂), 2.73 (4H, br. s, CH₂), |
| | 2.54 (2H, d, CH₂), 1.93-1.89 (1H, m, CH), 0.92 (6H, d, CH₃). |
| A-91 | ¹H NMR δ (ppm) (MeOH-d4): 9.10 (1H, ~d, ArH), 7.87-7.84 (1H, m, |
| | ArH), 7.74-7.71 (1H, m, ArH), 7.57 (1H, d, ArH), 7.02 (1H, s, ArH), 6.89 |
| | (1H, ~dd, ArH), 3.99 (2H, s, CH₂), 2.90 (4H, ~t, CH₂), 2.71 (4H, br. s, |
| | CH₂), 1.99-1.94 (1H, m, CH), 1.06-1.01 (2H, m, CH₂), 0.78-0.73 (2H, m, |
| | CH₂). |
| A-92 | ¹H NMR δ (ppm) (MeOH-d4): 7.62 (1H, d, ArH), 7.10 (1H, s, ArH), 7.04 |
| | (1H, d, ArH), 3.85 (2H, s, CH₂), 2.86 (4H, t, CH₂), 2.67 (4H, br. s, CH₂), |
| | 2.54 (5H, br. s, CH₃, CH₂), 1.94-1.87 (1H, m, CH), 0.92 (6H, d, CH₃). |
| A-93 | ¹H NMR δ (ppm) (MeOH-d4): 8.71 (1H, s, ArH), 8.60 (1H, d, ArH), 8.54 |
| | (1H, d, ArH), 7.61 (1H, d, ArH), 7.07 (1H, s, ArH), 7.02 (1H, d, ArH), |
| | 3.95 (2H, s, CH₂), 2.94 (4H, ~t, CH₂), 2.80 (4H, br. s, CH₂), 2.53 (2H, d, |
| | CH₂), 1.92-1.88 (1H, m, CH), 0.91 (6H, d, CH₃). |
| A-94 | ¹H NMR δ (ppm) (MeOH-d4): 8.42 (1H, s, ArH), 7.62 (1 H, d, ArH), 7.08 |
| | (1H, s, ArH), 7.03 (1H, d, ArH), 3.78 (5H, s, CH₃, CH₂), 2.85 (4H, t, |
| | CH₂), 2.57 (4H, br. s, CH₂), 2.53 (2H, d, CH₂), 1.94-1.87 (1H, m, CH), |
| | 0.92 (6H, d, CH₃). |
| A-95 | ¹H NMR δ (ppm) (MeOH-d4): 7.59 (1H, d, ArH), 7.07 (1H, s, ArH), 7.02 |
| | (1H, d, ArH), 6.37 (1H, s, ArH), 3.60 (2H, s, CH₂), 2.95 (4H, ~t, CH₂), |
| | 2.78 (4H, br. s, CH₂), 2.54 (2H, d, CH₂), 2.38 (3H, s, CH₃), 1.93-1.89 (1H, |
| | m, CH), 0.93 (6H, d, CH₃). |
| A-96 | ¹H NMR δ (ppm) (MeOH-d4): 7.62 (1H, d, ArH), 7.11 (1H, s, ArH), 7.05 |
| | (1H, d, ArH), 3.90 (2H, s, CH₂), 2.88 (4H, t, CH₂), 2.70 (4H, br. s, CH₂), |
| | 2.54 (2H, d, CH₂), 2.37 (3H, s, CH₃), 1.95-1.89 (1H, m, CH), 0.92 (6H, d, |
| | CH₃). |
| A-97 | ¹H NMR δ (ppm) (MeOH-d4): 7.72 (1H, d, ArH), 7.62 (1H, dd, ArH), |
| | 7.56 (1H, d, ArH), 7.11 (1H, s, ArH), 7.04 (1H, d, ArH), 3.97 (2H, s, |
| | CH₂), 2.90 (4H, ~t, CH₂), 2.71 (4H, br. s, CH₂), 2.54 (2H, d, CH₂), 1.93- |
| | 1.89 (1H, m, CH), 0.92 (6H, d, CH₃). |
| A-98 | ¹H NMR δ (ppm) (MeOH-d4): 7.92 (1H, d, ArH), 7.61 (1H, d, ArH), 7.16 |
| | (1H, s, ArH), 7.09 (1H, s, ArH), 7.04 (1H, d, ArH), 3.80 (2H, s, CH₂), |
| | 2.88 (4H, t, CH₂), 2.67 (4H, br. s, CH₂), 2.53 (2H, d, CH₂), 1.92-1.89 (1H, |
| | m, CH), 0.92 (6H, d, CH₃). |
| A-99 | ¹H NMR δ (ppm) (MeOH-d4): 8.66 (2H, s, ArH), 7.61 (1H, d, ArH), 7.09 |
| | (1H, s, ArH), 7.03 (1H, d, ArH), 3.68 (2H, s, CH₂), 2.90 (4H, ~t, CH₂), |
| | 2.67 (7H, br. s, CH₃, CH₂), 2.54 (2H, d, CH₂), 1.92-1.89 (1H, m, CH), |
| | 0.92 (6H, d, CH₃). |
| A-100 | ¹H NMR δ (ppm) (MeOH-d4): 7.63 (1H, d, ArH), 7.12 (1H, s, ArH), 7.06- |
| | 7.03 (1H, m, ArH), 6.18 (1H, s, ArH), 3.55 (2H, s, CH₂), 2.91 (4H, ~t, |
| | CH₂), 2.67 (4H, br. s, CH₂), 2.55 (2H, d, CH₂), 2.27 (3H, s, CH₃), 1.93- |
| | 1.90 (1H, m, CH), 0.92 (6H, d, CH₃). |
| A-101 | ¹H NMR δ (ppm) (MeOH-d4): 7.59 (1H, d, ArH), 7.05-7.01 (2H, m, |
| | ArH), 3.59 (2H, s, CH₂), 2.92 (4H, ~t, CH₂), 2.75 (4H, br. s, CH₂), 2.53 |
| | (2H, d, CH₂), 2.39 (3H, s, CH₃), 2.26 (3H, s, CH₃), 1.91-1.88 (1H, m, |
| | CH), 0.91 (6H, d, CH₃). |
| A-102 | ¹H NMR δ (ppm) (MeOH-d4): 7.61 (1H, d, ArH), 7.09 (1H, s, ArH), 7.04 |
| | (1H, d, ArH), 6.11 (1H, s, ArH), 3.74 (2H, s, CH₂), 2.91 (4H, ~t, CH₂), |
| | 2.73 (4H, br. s, CH₂), 2.54 (2H, d, CH₂), 2.12-2.07 (1H, m, CH), 1.92- |
| | 1.89 (1H, m, CH), 1.11-1.07 (2H, m, CH₂), 0.91 (8H, d, CH₃, CH₂). |
| A-103 | ¹H NMR δ (ppm) (MeOH-d4): 8.66 (2H, s, ArH), 7.61 (1H, d, ArH), 7.08 |
| | (1H, s, ArH), 7.03 (1H, d, ArH), 3.68 (2H, s, CH₂), 2.89 (4H, ~t, CH₂), |
| | 2.67 (7H, br. s, CH₃, CH₂), 2.53 (2H, d, CH₂), 1.92-1.88 (1H, m, CH), |
| | 0.91 (6H, d, CH₃). |
| A-104 | ¹H NMR δ (ppm) (MeOH-d4): 7.61 (1H, d, ArH), 7.08 (1 H, s, ArH), 7.03 |
| | (1 H, d, ArH), 6.02 (1H, s, ArH), 3.78 (3H, s, CH₃), 3.65 (2H, s, CH₂), |
| | 2.89 (4H, t, CH₂), 2.65 (4H, br. s, CH₂), 2.53 (2H, d, CH₂), 2.16 (3H, s, |
| | CH₃), 1.92-1.88 (1H, m, CH), 0.91 (6H, d, CH₃). |
| A-105 | ¹H NMR δ (ppm) (MeOH-d4): 7.95 (1H, s, ArH), 7.52 (1 H, d, ArH), |
| | 7.06.-6.96 (3H, m, ArH), 3.77 (3H, s, CH₃), 3.64 (2H, s, CH₂), 2.85 (4H, |
| | t, CH₂), 2.58 (4H, br. s, CH₂), 2.51 (2H, d, CH₂), 1.91-1.87 (1H, m, CH), |
| | 0.91 (6H, d, CH₃). |
| A-106 | ¹H NMR δ (ppm) (MeOH-d₄): 7.61 (1H, d, ArH), 7.36 (1H, d, ArH), 7.10 |
| | (1H, s, ArH), 7.04 (1H, d, ArH), 3.88 (2H, s, CH₂), 2.89 (4H, t, CH₂), |
| | 2.70 (4H, br. s, CH₂), 2.54 (2H, d, CH₂), 2.45 (3H, s, CH₃), 1.94-1.88 (1H, |
| | m, CH), 0.92 (6H, d, CH₃). |
| A-107 | ¹H NMR δ (ppm) (MeOH-d₄): 8.00 (1H, t, ArH), 7.82-7.77 (2H, m, ArH), |
| | 7.59 (1H, d, ArH), 7.07 (1H, s, ArH), 7.02 (1H, d, ArH), 3.96 (2H, s, |
| | CH₂), 2.95 (4H, br. s, CH₂), 2.81 (4H, br. s, CH₂), 2.54 (2H, d, CH₂), |
| | 1.94-1.87 (1H, m, CH), 0.92 (6H, d, CH₃). |
| A-108 | ¹H NMR δ (ppm) (MeOH-d4): 9.24 (1H, s, ArH), 7.62 (1H, d, ArH), 7.10 |
| | (1H, s, ArH), 7.04 (1H, d, ArH), 3.84 (2H, s, CH₂), 2.89 (4H, ~t, CH₂), |
| | 2.71 (4H, br. s, CH₂), 2.54 (2H, d, CH₂), 1.92-1.89 (1H, m, CH), 0.92 |
| | (6H, d, CH₃). |
| A-109 | ¹H NMR δ (ppm) (MeOH-d₄): 7.69 (1H, s, ArH), 7.47 (1H, s, ArH), 7.26 |
| | (1H, s, ArH), 6.91 (2H, br. s, ArH), 4.06 (2H, s, CH₂), 3.74 (3H, s, CH₃), |
| | 3.10 (4H, br. s, CH₂), 2.99 (4H, br. s, CH₂), 2.49 (2H, d, CH₂), 1.91-1.87 |
| | (1H, m, CH), 0.91 (6H, d, CH₃). |
| A-110 | ¹H NMR δ (ppm) (MeOH-d₄): 7.61 (1H, d, ArH), 7.51 (1H, d, ArH), 6.98- |
| | 6.95 (2H, m, ArH), 6.37 (1H, d, ArH), 4.10 (2H, s, CH₂), 3.89 (3H, s, |
| | CH₃), 3.10 (4H, br. s, CH₂), 3.00 (4H, ~d, CH₂), 2.51 (2H, d, CH₂), 1.91- |
| | 1.88 (1H, m, CH), 0.91 (6H, d, CH₃). |
| A-111 | ¹H NMR δ (ppm) (MeOH-d₄): 8.82 (1H, s, ArH), 7.53 (1H, d, ArH), 6.98 |
| | (2H, d, ArH), 4.15 (2H, s, CH₂), 3.06 (4H, br. s, CH₂), 2.99 (4H, br. s, |
| | CH₂), 2.53 (3H, s, CH₃), 2.51 (2H, d, CH₂), 1.91-1.88 (1H, m, CH), 0.91 |
| | (6H, d, CH₃). |
| A-112 | ¹H NMR δ (ppm) (MeOH-d₄): 8.98 (1H, d, ArH), 7.68 (1H, s, ArH), 7.61 |
| | (1H, d, ArH), 7.08 (1H, s, ArH), 7.03 (1H, d, ArH), 3.96 (2H, s, CH₂), |
| | 2.92 (4H, ~t, CH₂), 2.73 (4H, br. s, CH₂), 2.53 (2H, d, CH₂), 2.41 (3H, s, |
| | CH₃), 1.92-1.89 (1H, m, CH), 0.92 (6H, d, CH₃). |
| A-113 | ¹H NMR δ (ppm) (CHCl₃-d): 8.24 (1H, d, ArH), 7.61 (1H, br. s, ArH), |
| | 7.34 (1H, d, ArH), 7.15 (1H, s, ArH), 7.12 (1H, d, ArH), 4.02 (2H, s, |
| | CH₂), 3.02 (4H, br. s, CH₂), 2.85 (4H, br. s, CH₂), 2.69 (3H, s, CH₃), 2.51 |
| | (2H, d, CH₂), 1.90-1.84 (1H, m, CH), 0.90 (6H, d, CH₃). |
| A-114 | ¹H NMR δ (ppm) (MeOH-d₄): 7.48 (1H, d, ArH), 7.20 (1H, s, ArH), 6.96 |
| | (1H, s, ArH), 6.94 (1H, s, ArH), 3.73 (3H, s, CH₃), 3.60 (2H, s, CH₂), |
| | 2.82 (4H, ~t, CH₂), 2.53-2.45 (9H, m, CH₃, CH₂), 1.92-1.85 (1H, m, CH), |
| | 0.95 (6H, d, CH₃). |
| A-115 | ¹H NMR δ (ppm) (MeOH-d₄): 7.68 (1H, d, ArH), 7.60 (1H, d, ArH), 7.18 |
| | (1H, d, ArH), 7.07 (1H, s, ArH), 7.02 (1H, d, ArH), 4.07 (3H, s, CH₃), |
| | 3.92 (2H, s, CH₂), 2.91 (4H, ~t, CH₂), 2.73 (4H, br. s, CH₂), 2.53 (2H, d, |
| | CH₂), 1.93-1.87 (1H, m, CH), 0.92 (6H, d, CH₃). |
| A-116 | ¹H NMR δ (ppm) (MeOH-d₄): 7.94 (1H, d, ArH), 7.89 (1H, d, ArH), 7.47 |
| | (1H, t, ArH), 7.39 (1H, t, ArH), 6.62-6.55 (2H, m, ArH), 3.98 (2H, s, |
| | NCH₂), 3.85 (3H, s, OCH₃), 2.86 (4H, ~t, CH₂), 2.60 (4H, br. s, CH₂). |
| A-117 | ¹H NMR δ (ppm) (MeOH-d₄): 7.95 (1H, d, ArH), 7.90 (1H, d, ArH), 7.53- |
| | 7.45 (2H, m, ArH), 7.42 (1H, t, ArH), 7.05 (1H, d, ArH), 4.06 (2H, s, |
| | NCH₂), 3.97 (3H, s, OCH₃), 2.94 (4H, ~t, CH₂), 2.78 (4H, br. s, CH₂). |
| A-118 | ¹H NMR δ (ppm) (MeOH-d₄): 7.94 (1H, d, ArH), 7.89 (1H, d, ArH), 7.47 |
| | (1H, t, ArH), 7.39 (1H, t, ArH), 6.59-6.52 (2H, m, ArH), 4.67 (1H, ~sept, |
| | CH), 3.99 (2H, s, NCH₂), 2.86 (4H, ~t, CH₂), 2.61 (4H, br. s, CH₂), 1.33 |
| | (6H, d, CH₃). |
| A-119 | ¹H NMR δ (ppm) (MeOH-d₄): 7.95 (1H, d, ArH), 7.90 (1H, d, ArH), 7.54 |
| | (1H, s, ArH), 7.48 (1H, t, ArH), 7.40 (1H, t, ArH), 6.87 (1H, s, ArH), 4.76 |
| | (1H, ~sept, CH), 4.04 (2H, s, NCH₂), 2.93 (4H, ~t, CH₂), 2.78 (4H, br. s, |
| | CH₂), 2.60 (2H, q, CH₂), 1.37 (6H, d, CH₃), 1.17 (3H, t, CH₃). |
| A-120 | ¹H NMR δ (ppm) (MeOH-d₄): 7.95 (1H, d, ArH), 7.89 (1H, d, ArH), 7.48 |
| | (1H, t, ArH), 7.40 (1H, t, ArH), 6.66 (1H, s, ArH), 6.63 (1H, s, ArH), 4.69 |
| | (1H, ~sept, CH), 3.97 (2H, s, NCH₂), 2.83 (4H, ~t, CH₂), 2.52 (4H, br. s, |
| | CH₂), 2.43 (2H, q, CH₂), 1.33 (6H, d, CH₃), 1.00 (3H, t, CH₃). |
| A-121 | ¹H NMR δ (ppm) (MeOH-d₄): 7.95 (1H, d, ArH), 7.90 (1H, d, ArH), 7.47 |
| | (1H, t, ArH), 7.40 (1H, t, ArH), 7.24 (1H, s, ArH), 6.87 (1H, s, ArH), 4.75 |
| | (1H, ~sept, OCH), 4.04 (2H, s, NCH₂), 2.92 (4H, ~t, CH₂), 2.78 (4H, br. s, |
| | CH₂), 2.14-2.06 (1H, m, CH), 1.39 (6H, d, CH₃), 0.93-0.86 (2H, m, CH₂), |
| | 0.66-0.58 (2H, m, CH₂). |
| A-122 | ¹H NMR δ (ppm) (MeOH-d₄): 7.94 (1H, d, ArH), 7.88 (1H, d, ArH), 7.47 |
| | (1H, t, ArH), 7.40 (1H, t, ArH), 6.61 (1H, s, ArH), 6.33 (1H, s, ArH), 4.66 |
| | (1H, ~sept, CH), 3.92 (2H, s, NCH₂), 2.84 (4H, ~t, CH₂), 2.48 (4H, br. s, |
| | CH₂), 1.71-1.63 (1H, m, CH), 1.31 (6H, d, CH₃), 0.76-0.70 (2H, m, CH₂), |
| | 0.57-0.52 (2H, m, CH₂). |
| A-123 | ¹H NMR δ (ppm) (MeOH-d₄): 7.95 (1H, d, ArH), 7.90 (1H, d, ArH), 7.47 |
| | (1H, t, ArH), 7.43-7.39 (2H, m, ArH), 7.00 (1H, s, ArH), 4.71 (1H, ~sept, |
| | OCH), 4.09 (2H, q, OCH₂), 4.06 (2H, s, NCH₂), 2.95 (4H, ~t, CH₂), 2.81 |
| | (4H, br. s, CH₂), 1.40 (3H, t, CH₃), 1.35 (6H, d, CH₃). |
| A-124 | ¹H NMR δ (ppm) (MeOH-d₄): 7.95 (1H, d, ArH), 7.90 (1H, d, ArH), 7.52- |
| | 7.36 (3H, m, ArH), 6.89 (1H, d, ArH), 4.06 (2H, s, NCH₂), 2.90 (4H, ~t, |
| | CH₂), 2.78 (4H, br. s, CH₂), 2.17-2.09 (1H, m, CH), 1.11-1.04 (2H, m, |
| | CH₂), 0.90-0.83 (2H, m, CH₂). |
| A-125 | ¹H NMR δ (ppm) (MeOH-d₄): 7.95 (1H, d, ArH), 7.90 (1H, d, ArH), 7.48 |
| | (1H, t, ArH), 7.40 (1H, t, ArH), 6.82 (1H, s, ArH), 6.63 (1H, d, ArH), |
| | 4.00 (2H, s, NCH₂), 2.88 (4H, ~t, CH₂), 2.61 (4H, br. s, CH₂), 2.02-1.96 |
| | (1H, m, CH), 1.09-1.04 (2H, m, CH₂) , 0.81-0.76 (2H, m, CH₂). |
| A-126 | ¹H NMR δ (ppm) (MeOH-d4): 7.95 (1H, d, ArH), 7.89 (1 H, d, ArH), |
| | 7.54 (1H, s, ArH), 7.48 (1H, t, ArH), 7.40 (1H, t, ArH), 6.92 (1H, s, ArH), |
| | 4.03 (2H, s, NCH₂), 2.91 (4H, ~t, CH₂), 2.76 (4H, br. s, CH₂), 2.42 (3H, s, |
| | CH₃), 2.01-1.92 (1H, m, CH), 1.05-1.00 (2H, m, CH₂), 0.73-0.68 (2H, m, |
| | CH₂). |
| A-127 | ¹H NMR δ (ppm) (MeOH-d4): 7.94 (1H, d, ArH), 7.88 (1 H, d, ArH), |
| | 7.47 (1H, t, ArH), 7.39 (1H, t, ArH), 6.84 (1H, s, ArH), 6.77 (1H, s, ArH), |
| | 3.93 (2H, s, NCH₂), 2.82 (4H, ~t, CH₂), 2.50 (4H, br. s, CH₂), 2.09 (3H, s, |
| | CH₃), 1.95-1.90 (1H, m, CH), 1.03-0.98 (2H, m, CH₂), 0.74-0.73 (2H, m, |
| | CH₂). |
| A-128 | ¹H NMR δ (ppm) (MeOH-d4): 7.94 (1H, d, ArH), 7.89 (1 H, d, ArH), |
| | 7.47 (1H, t, ArH), 7.39 (1H, t, ArH), 6.84 (1H, s, ArH), 6.82 (1H, s, ArH), |
| | 3.92 (2H, s, NCH₂), 2.83 (4H, ~t, CH₂), 2.47 (4H, br. s, CH₂), 2.40 (2H, q, |
| | CH₂), 1.97-1.92 (1H, m, CH), 1.04-0.96 (5H, m, CH₃, CH₂), 0.77-0.72 |
| | (2H, m, CH₂). |
| A-129 | ¹H NMR δ (ppm) (MeOH-d4): 7.94 (1H, d, ArH), 7.89 (1 H, d, ArH), |
| | 7.48 (1H, t, ArH), 7.40 (1H, t, ArH), 6.81 (1H, s, ArH), 6.54 (1H, s, ArH), |
| | 3.96 (2H, s, NCH₂), 2.86 (4H, ~t, CH₂), 2.51 (4H, br. s, CH₂), 1.95-1.90 |
| | (1H, m, CH), 1.64-1.60 (1H, m, CH), 1.03-0.96 (2H, m, CH₂), 0.74-0.67 |
| | (4H, m, CH₂), 0.57-0.51 (2H, m, CH₂). |
| A-130 | ¹H NMR δ (ppm) (MeOH-d₄): 7.98 (1H, d, ArH), 7.93 (1H, d, ArH), 7.50 |
| | (1H, t, ArH), 7.42 (1H, t, ArH), 7.30-7.27 (2H, m, ArH), 4.14 (2H, s, |
| | NCH₂), 3.37-3.23 (6H, m, CH₂), 3.00 (4H, br. s, CH₂), 1.75-1.67 (1H, m, |
| | CH), 1.29 (3H, dd, CH₃), 1.04-0.99 (2H, m, CH₂), 0.66-0.62 (2H, m, |
| | CH₂). |
| A-131 | ¹H NMR δ (ppm) (DMSO-d₆): 9.45 (1H, s, NH), 8.02 (1H, d, ArH), 7.89 |
| | (1H, d, ArH), 7.63 (1H, s, ArH), 7.45 (1H, t, ArH), 7.37 (1H, t, ArH), |
| | 6.69 (1H, s, ArH), 3.95 (2H, s, NCH₂), 2.75 (4H, br. s, CH₂), 2.62 (4H, br. |
| | s, CH₂), 2.04 (4H, br. s, CH, CH₃), 0.99-0.93 (2H, m, CH₂), 0.74-0.68 |
| | (2H, m, CH₂). |
| A-132 | ¹H NMR δ (ppm) (MeOH-d4): 7.94 (1H, d, ArH), 7.89 (1 H, d, ArH), |
| | 7.47 (1H, t, ArH), 7.39 (1H, t, ArH), 6.87 (1H, s, ArH), 6.80 (1H, ~d, |
| | ArH), 4.00 (2H, s, NCH₂), 2.89 (4H, br. s, CH₂), 2.62 (4H, br. s, CH₂), |
| | 2.54 (2H, d, CH₂), 1.94-1.90 (1H, m, CH), 0.93 (6H, d, CH₃). |
| A-133 | ¹H NMR δ (ppm) (MeOH-d4): 7.96 (1H, d, ArH), 7.91 (1 H, d, ArH), |
| | 7.52-7.36 (3H, m, ArH), 7.21 (1H, ~d, ArH), 4.07 (2H, s, NCH₂), 2.92 |
| | (4H, br. s, CH₂), 2.79 (4H, br. s, CH₂), 2.59 (2H, d, CH₂), 1.98-1.90 (1H, |
| | m, CH), 0.95 (6H, d, CH₃). |
| A-134 | ¹H NMR δ (ppm) (MeOH-d4): 7.95 (1H, d, ArH), 7.90 (1 H, d, ArH), |
| | 7.54 (1H, s, ArH), 7.48 (1H, t, ArH), 7.40 (1H, t, ArH), 7.09 (1H, s, ArH), |
| | 4.04 (2H, s, NCH₂), 2.92 (4H, ~t, CH₂), 2.77 (4H, br. s, CH₂), 2.56 (2H, d, |
| | CH₂), 2.31 (3H, s, CH₃), 1.93-1.86 (1H, m, CH), 0.96 (6H, d, CH₃). |
| A-135 | ¹H NMR δ (ppm) (MeOH-d4): 7.94 (1H, d, ArH), 7.88 (1 H, d, ArH), |
| | 7.47 (1H, t, ArH), 7.39 (1H, t, ArH), 6.90-6.89 (2H, m, ArH), 3.92 (2H, s, |
| | NCH₂), 2.83 (4H, ~t, CH₂), 2.50-2.48 (6H, m, CH₂), 2.09 (3H, s, CH₃), |
| | 1.93-1.88 (1H, m, CH), 0.93-0.91 (6H, d, CH₃). |
| A-136 | ¹H NMR δ (ppm) (MeOH-d4): 7.94 (1H, d, ArH), 7.88 (1 H, d, ArH), |
| | 7.47 (1H, t, ArH), 7.39 (1H, t, ArH), 6.93 (2H, s, ArH), 3.93 (2H, s, |
| | NCH₂), 2.84 (4H, ~t, CH₂), 2.54-2.36 (8H, m, CH₂), 1.93-1.86 (1H, m, |
| | CH), 0.98 (3H, t, CH₃), 0.93 (6H, d, CH₃). |
| A-137 | ¹H NMR δ (ppm) (MeOH-d4): 7.94 (1H, d, ArH), 7.88 (1 H, d, ArH), |
| | 7.47 (1H, t, ArH), 7.39 (1H, t, ArH), 6.91 (1H, s, ArH), 6.63 (1H, s, ArH), |
| | 3.92 (2H, s, NCH₂), 2.86 (4H, ~t, CH₂), 2.51-2.47 (6H, m, CH₂), 1.91- |
| | 1.82 (1H, m, CH), 1.66-1.61 (1H, m, CH), 0.93-0.88 (6H, d, CH₃), 0.76- |
| | 0.69 (2H, m, CH₂), 0.58-0.52 (2H, m, CH₂). |
| A-138 | ¹H NMR δ (ppm) (MeOH-d4): 7.97 (1H, d, ArH), 7.92 (1 H, d, ArH), |
| | 7.49 (1H, t, ArH), 7.41 (1H, t, ArH), 7.32 (1H, s, ArH), 7.23 (1H, s, ArH), |
| | 4.12 (2H, s, NCH₂), 3.24 (4H, ~t, CH₂), 2.95 (4H, br. s, CH₂), 2.47 (2H, d, |
| | CH₂), 2.03-1.96 (1H, m, CH), 0.97 (6H, d, CH₃). |
| A-139 | ¹H NMR δ (ppm) (MeOH-d4): 7.98 (1H, d, ArH), 7.93 (1 H, d, ArH), |
| | 7.49 (1H, t, ArH), 7.42 (1H, t, ArH), 7.28 (1H, s, ArH), 7.24 (1H, s, ArH), |
| | 4.14 (2H, s, NCH₂), 3.33 (4H, ~t, CH₂), 3.00 (4H, br. s, CH₂), 2.88 (3H, s, |
| | CH₃), 2.47 (2H, d, CH₂), 2.01-1.94 (1H, m, CH), 0.95 (6H, d, CH₃). |
| A-140 | ¹H NMR δ (ppm) (MeOH-d4): 7.97 (1H, d, ArH), 7.92 (1 H, d, ArH), |
| | 7.49 (1H, t, ArH), 7.42 (1H, t, ArH), 7.28 (2H, s, ArH), 4.13 (2H, s, |
| | NCH₂), 3.34-3.22 (6H, m, CH₂), 2.99 (4H, br. s, CH₂), 2.48 (2H, d, CH₂), |
| | 2.03-1.93 (1H, m, CH), 1.26 (3H, t, CH₃), 0.96 (6H, d, CH₃). |
| A-141 | ¹H NMR δ (ppm) (MeOH-d4): 8.24 (1H, br. s, ArH), 7.95 (1H, d, ArH), |
| | 7.91 (1H, d, ArH), 7.59 (1H, s, ArH), 7.48 (1H, t, ArH), 7.40 (1H, t, |
| | ArH), 4.10 (2H, s, NCH₂), 3.06 (4H, br. s, CH₂), 2.87 (4H, br. s, CH₂), |
| | 2.75 (2H, q, CH₂), 1.30 (3H, t, CH₃). |
| A-142 | ¹H NMR δ (ppm) (MeOH-d4): 7.95 (1H, d, ArH), 7.91 (1H, d, ArH), 7.86 |
| | (1H, d, ArH), 7.47 (1H, t, ArH), 7.41 (1H, t, ArH), 6.94 (1H, d, ArH), |
| | 4.09 (2H, s, NCH₂), 3.19 (4H, ~t, CH₂), 2.80-2.73 (6H, m, CH₂), 1.29 |
| | (3H, t, CH₃). |
| A-143 | ¹H NMR δ (ppm) (MeOH-d4): 8.14 (1H, br. s, ArH), 7.97-7.89 (2H, m, |
| | ArH), 7.48 (1H, t, ArH), 7.40 (1H, t, ArH), 7.33 (1H, s, ArH), 4.10 (2H, s, |
| | NCH₂), 3.04 (4H, br. s, CH₂), 2.87 (4H, br. s, CH₂), 2.08-1.98. (1H, m, |
| | CH), 1.15-1.08 (2H, m, CH₂), 0.86 (2H, br. s, CH₂). |
| A-144 | ¹H NMR δ (ppm) (MeOH-d₄): 8.08 (1H, s, ArH), 7.94 (1H, d, ArH), 7.89 |
| | (1H, d, ArH), 7.47 (1H, t, ArH), 7.39 (1H, t, ArH), 6.92 (1H, s, ArH), |
| | 3.99 (2H, s, NCH₂), 3.24 (4H, ~t, CH₂), 2.67 (4H, br. s, CH₂), 2.17-2.13 |
| | (1H, m, CH), 1.28-1.22 (2H, m, CH₂), 1.15-1.12 (2H, m, CH₂). |
| A-145 | ¹H NMR δ (ppm) (MeOH-d4): 7.95 (1H, d, ArH), 7.90 (1H, d, ArH), 7.81 |
| | (1H, d, ArH), 7.48 (1H, t, ArH), 7.40 (1H, t, ArH), 6.98 (1H, d, ArH), |
| | 4.08 (2H, s, NCH₂), 3.14 (4H, ~t, CH₂), 2.77 (4H, ~t, CH₂), 2.07-2.02 |
| | (1H, m, CH), 1.07-1.04 (2H, m, CH₂), 1.00-0.95 (2H, m, CH₂). |
| A-146 | ¹H NMR δ (ppm) (DMSO-d6): 8.21 (1H, s, ArH), 8.03 (1H, d, ArH), 7.90 |
| | (1H, d, ArH), 7.45 (1H, t, ArH), 7.37 (1H, t, ArH), 3.97 (2H, s, NCH₂), |
| | 3.24 (4H, br. s, CH₂), 2.64 (4H, br. s, CH₂), 2.21-2.11 (1H, m, CH), 1.08- |
| | 0.97 (4H, m, CH₂). |
| A-147 | ¹H NMR δ (ppm) (MeOH-d4): 8.21 (1H, br. s, ArH), 7.96 (1H, d, ArH), |
| | 7.92 (1H, d, ArH), 7.55 (1H, s, ArH), 7.49 (1H, t, ArH), 7.43 (1H, t, |
| | ArH), 4.17 (2H, s, NCH₂), 3.09 (4H, br. s, CH₂), 2.94 (4H, br. s, CH₂), |
| | 2.60 (2H, d, CH₂), 2.00-1.88 (1H, m, CH), 0.95 (6H, d, CH₃). |
| A-148 | ¹H NMR δ (ppm) (MeOH-d4): 7.96 (1H, d, ArH), 7.92 (1H, d, ArH), 7.84 |
| | (1H, s, ArH), 7.49 (1H, t, ArH), 7.42 (1H, t, ArH), 6.90 (1H, d, ArH), |
| | 4.10 (2H, s, NCH₂), 3.18 (4H, br. s, CH₂), 2.79 (4H, br. s, CH₂), 2.61 (2H, |
| | d, CH₂), 2.17-2.12 (1H, m, CH), 0.93 (6H, d, CH₃). |
| A-149 | ¹H NMR δ (ppm) (MeOH-d4): 7.95 (1H, d, ArH), 7.90 (1 H, d, ArH), |
| | 7.77 (1H, s, ArH), 7.48 (1H, t, ArH), 7.40 (1H, t, ArH), 6.78 (1H, s, ArH), |
| | 4.06 (2H, s, NCH₂), 3.04 (2H, d, CH₂), 3.01 (4H, ~t, CH₂), 2.83 (4H, br. s, |
| | CH₂), 1.14-1.07 (1H, m, CH), 0.60-0.54 (2H, m, CH₂), 0.30-0.26 (2H, m, |
| | CH₂). |
| A-150 | ¹H NMR δ (ppm) (DMSO-d6): 11.91 (1H, br. s, NH), 8.06 (1H, d, ArH), |
| | 7.76 (1H, ~t, ArH), 7.60 (1H, d, ArH), 7.46 (1H, t, ArH), 6.68 (1H, d, |
| | ArH), 6.66 (1H, s, ArH), 3.79 (3H, s, OCH₃), 3.38 (2H, s, NCH₂), 2.72 |
| | (4H, ~t, CH₂), 2.39 (4H, br. s, CH₂). |
| A-151 | ¹H NMR δ (ppm) (MeOH-d4): 8.18 (1H, ~dd, ArH), 7.81 (1H, ~t, ArH), |
| | 7.66 (1H, d, ArH), 7.52 (1H, t, ArH), 7.50-6.54 (2H, m, ArH), 4.67 (1H, ~sept, |
| | OCH), 3.59 (2H, s, NCH₂), 2.87 (4H, ~t, CH₂), 2.59 (4H, br. s, |
| | CH₂), 1.33 (6H, d, CH₃). |
| A-152 | ¹H NMR δ (ppm) (MeOH-d4): 8.18 (1H, ~dd, ArH), 7.79 (1H, ~t, ArH), |
| | 7.65 (1H, ~d, ArH), 7.51 (1H, ~t, ArH), 6.60 (1H, s, ArH), 6.32 (1H, s, |
| | ArH), 4.65 (1H, ~sept, OCH), 3.50 (2H, s, NCH₂), 2.84 (4H, ~t, CH₂), |
| | 2.43 (4H, br. s, CH₂), 1.69-1.63 (1H, m, CH), 1.30 (6H, d, CH₃), 0.74- |
| | 0.71 (2H, m, CH₂), 0.57-0.51 (2H, m, CH₂). |
| A-153 | ¹H NMR δ (ppm) (DMSO-d6): 11.91 (1H, br. s, NH), 8.07 (1H, d, ArH), |
| | 7.77 (1H, ~t, ArH), 7.61 (1H, d, ArH), 7.49-7.40 (2H, m, ArH), 7.14 (1H, |
| | d, ArH), 3.46 (2H, s, NCH₂), 2.73 (4H, ~t, CH₂), 2.57 (4H, br. s, CH₂), |
| | 2.56-2.47 (2H, m, CH₂), 1.90-1.82 (1H, m, CH), 0.87 (6H, d, CH₃). |
| A-154 | ¹H NMR δ (ppm) (DMSO-d6): 11.89 (1H, br. s, NH), 8.06 (1H, d, ArH), |
| | 7.76 (1H, ~t, ArH), 7.60 (1H, d, ArH), 7.46 (1H, t, ArH), 6.87-6.81 (2H, |
| | m, ArH), 3.38 (2H, s, NCH₂), 2.73 (4H, ~t, CH₂), 2.47 (2H, br. s, CH₂), |
| | 2.38 (4H, br. s, CH₂), 1.91-1.83 (1H, m, CH), 0.85 (6H, d, CH₃). |
| A-155 | ¹H NMR δ (ppm) (MeOH-d₄): 9.08 (1H, d, ArH), 7.82-7.79 (1H, m, ArH), |
| | 7.73-7.69 (1H, m, ArH), 6.56-6.50 (2H, m, ArH), 3.94 (2H, s, NCH₂), |
| | 3.83 (3H, s, OCH₃), 2.85 (4H, ~t, CH₂), 2.56 (4H, br. s, CH₂). |
| A-156 | ¹H NMR δ (ppm) (MeOH-d₄): 9.09 (1H, d, ArH), 7.82-7.79 (1H, m, ArH), |
| | 7.73-7.69 (1H, m, ArH), 6.53 (1H, d, ArH), 6.49 (1H, s, ArH), 4.65 (1H, ~sept, |
| | CH), 3.96 (2H, s, NCH₂), 2.85 (4H, ~t, CH₂), 2.59 (4H, br. s, CH₂), |
| | 1.32 (6H, d, CH₃). |
| A-157 | ¹H NMR δ (ppm) (MeOH-d₄): 9.12 (1H, d, ArH), 7.87-7.83 (1H, m, ArH), |
| | 7.76-7.72 (1H, m, ArH), 7.34 (1H, ~dd, ArH), 7.03 (1H, t, ArH), 4.68 |
| | (1H, ~sept, CH), 4.07 (2H, s, NCH₂), 3.17 (4H, br. s, CH₂), 2.73 (4H, br. |
| | s, CH₂), 1.35 (6H, d, CH₃). |
| A-158 | ¹H NMR δ (ppm) (MeOH-d₄): 9.10 (1H, d, ArH), 7.87-7.83 (1H, m, ArH), |
| | 7.74-7.70 (1H, m, ArH), 7.52 (1H, s, ArH), 6.83 (1H, s, ArH), 4.73 (1H, ~sept, |
| | OCH), 3.96 (2H, s, NCH₂), 2.90 (4H, ~t, CH₂), 2.71 (4H, br. s, |
| | CH₂), 2.59 (2H, q, CH₂), 1.35 (6H, d, CH₃), 1.16 (3H, t, CH₃). |
| A-159 | ¹H NMR δ (ppm) (MeOH-d₄): 9.09 (1H, d, ArH), 7.82-7.78 (1H, m, ArH), |
| | 7.73-7.68 (1H, m, ArH), 6.65 (1H, ~d, ArH), 6.59 (1H, ~d, ArH), 4.67 |
| | (1H, ~sept, OCH), 3.87 (2H, s, NCH₂), 2.80 (4H, ~t, CH₂), 2.44 (4H, br. s, |
| | CH₂), 2.41 (2H, q, CH₂), 1.32 (6H, d, CH₃), 0.99 (3H, t, CH₃). |
| A-160 | ¹H NMR δ (ppm) (MeOH-d₄): 9.10 (1H, d, ArH), 7.87-7.83 (1H, m, ArH), |
| | 7.74-7.70 (1H, m, ArH), 7.22 (1H, s, ArH), 6.83 (1H, s, ArH), 4.72 (1H, ~sept, |
| | OCH), 3.97 (2H, s, NCH₂), 2.90 (4H, ~t, CH₂), 2.71 (4H, br. s, |
| | CH₂), 2.14-2.07 (1H, m, CH), 1.37 (6H, d, CH₃), 0.91-0.85 (2H, m, CH₂), |
| | 0.64-0.60 (2H, m, CH₂). |
| A-161 | ¹H NMR δ (ppm) (MeOH-d₄): 9.08 (1H, ~d, ArH), 7.82-7.78 (1H, m, |
| | ArH), 7.72-7.68 (1H, m, ArH), 6.57 (1H, s, ArH), 6.31 (1H, s, ArH), 4.64 |
| | (1H, ~sept, OCH), 3.87 (2H, s, NCH₂), 2.82 (4H, ~t, CH₂), 2.44 (4H, br. s, |
| | CH₂), 1.69-1.63 (1H, m, CH), 1.31 (6H, d, CH₃), 0.75-0.70 (2H, m, CH₂), |
| | 0.56-0.52 (2H, m, CH₂). |
| A-162 | ¹H NMR δ (ppm) (MeOH-d₄): 9.10 (1H, d, ArH), 7.88-7.84 (1H, m, ArH), |
| | 7.75-7.71 (1H, m, ArH), 7.39 (1H, s, ArH), 6.96 (1H, s, ArH), 4.69 (1H, ~sept, |
| | OCH), 4.07 (2H, q, OCH₂), 4.00 (2H, s, NCH₂), 2.95 (4H, ~t, CH₂), |
| | 2.77 (4H, br. s, CH₂), 1.39 (3H, t, CH₃) , 1.34 (6H, d, CH₃). |
| A-163 | ¹H NMR δ (ppm) (MeOH-d4): 9.12 (1H, ~d, ArH), 7.86-7.83 (1H, m, |
| | ArH), 7.75-7.71 (1H, m, ArH), 7.42 (1H, d, ArH), 7.14 (1H, ~d, ArH), |
| | 4.09 (2H, s, NCH₂), 2.92 (4H, br. s, CH₂), 2.82 (4H, br. s, CH₂), 2.57 (2H, |
| | d, CH₂), 1.95-1.90 (1H, m, CH), 0.94 (6H, d, CH₃). |
| A-164 | ¹H NMR δ (ppm) (MeOH-d4): 9.11 (1H, ~d, ArH), 7.82-7.78 (1H, m, |
| | ArH), 7.74-7.69 (1H, m, ArH), 6.83-6.76 (2H, m, ArH), 4.06 (2H, s, |
| | NCH₂), 2.91 (4H, br. s, CH₂), 2.68 (4H, br. s, CH₂), 2.52 (2H, d, CH₂), |
| | 1.93-1.88 (1H, m, CH), 0.92 (6H, d, CH₃). |
| A-165 | ¹H NMR δ (ppm) (MeOH-d4): 9.13 (1H, ~d, ArH), 7.86-7.83 (1H, m, |
| | ArH), 7.77-7.73 (1H, m, ArH), 7.32 (1H, d, ArH), 7.15 (1H, ~t, ArH), |
| | 4.15 (2H, s, NCH₂), 3.18 (4H, br. s, CH₂), 2.80 (4H, br. s, CH₂), 2.57 (2H, |
| | d, CH₂), 1.94-1.89 (1H, m, CH), 0.94 (6H, d, CH₃). |
| A-166 | ¹H NMR δ (ppm) (MeOH-d4): 9.09 (1H, ~d, ArH), 7.87-7.83 (1H, m, |
| | ArH), 7.74-7.70 (1H, m, ArH), 7.49 (1H, s, ArH), 7.03 (1H, s, ArH), 3.96 |
| | (2H, s, NCH₂), 2.90 (4H, ~t, CH₂), 2.69 (4H, br. s, CH₂), 2.54 (2H, d, |
| | CH₂), 2.29 (3H, s, CH₃), 1.90-1.85 (1H, m, CH), 0.94 (6H, d, CH₃). |
| A-167 | ¹H NMR δ (ppm) (MeOH-d4): 9.09 (1H, ~d, ArH), 7.82-7.78 (1H, m, |
| | ArH), 7.73-7.68 (1H, m, ArH), 6.90-6.88 (2H, m, ArH), 3.91 (2H, s, |
| | NCH₂), 2.83 (4H, ~t, CH₂), 2.50-2.46 (6H, m, CH₂), 2.09 (3H, s, CH₃), |
| | 1.92-1.84 (1H, m, CH), 0.91 (6H, d, CH₃). |
| A-168 | ¹H NMR δ (ppm) (MeOH-d4): 9.09 (1H, ~d, ArH), 7.81-7.79 (1 H, m, |
| | ArH), 7.72-7.69 (1H, m, ArH), 6.92 (1H, s, ArH), 6.90 (1H, s, ArH), 3.90 |
| | (2H, s, NCH₂), 2.83 (4H, br. s, CH₂), 2.52-2.36 (8H, m, CH₂), 1.91-1.87 |
| | (1H, m, CH), 0.99 (3H, t, CH₃), 0.91 (6H, d, CH₃). |
| A-169 | ¹H NMR δ (ppm) (MeOH-d4): 9.09 (1H, ~d, ArH), 7.82-7.79 (1 H, m, |
| | ArH), 7.73-7.69 (1H, m, ArH), 6.87 (1H, s, ArH), 6.61 (1H, s, ArH), 3.90 |
| | (2H, s, NCH₂), 2.90 (4H, ~t, CH₂), 2.55-2.46 (6H, m, CH₂), 1.89-1.82 |
| | (1H, m, CH), 1.62-1.59 (1H, m, CH), 0.89 (6H, d, CH₃), 0.74-0.71 (2H, |
| | m, CH₂), 0.55-0.54 (2H, m, CH₂). |
| A-170 | ¹H NMR δ (ppm) (MeOH-d4): 9.11 (1H, ~d, ArH), 7.93-7.89 (1H, m, |
| | ArH), 7.76-7.72 (1H, m, ArH), 7.25 (2H, s, ArH), 4.03 (2H, s, NCH₂), |
| | 3.33 (4H, ~t, CH₂), 2.93 (4H, br. s, CH₂), 2.88 (3H, s, CH₃), 2.45 (2H, d, |
| | CH₂), 1.98-1.94 (1H, m, CH), 0.94 (6H, d, CH₃). |
| A-171 | ¹H NMR δ (ppm) (MeOH-d4): 9.09 (1H, ~d, ArH), 7.87-7.84 (1H, m, |
| | ArH), 7.74-7.70 (1H, m, ArH), 6.43 (1H, s, ArH), 6.38 (1H, s, ArH), 3.93 |
| | (2H, s, NCH₂), 2.94 (4H, br. s, CH₂), 2.84 (3H, s, CH₃), 2.67 (4H, br. s, |
| | CH₂), 2.45 (2H, d, CH₂), 1.93-1.88 (1H, m, CH), 0.93 (6H, d, CH₃). |
| A-172 | ¹H NMR δ (ppm) (MeOH-d4): 9.11 (1H, ~d, ArH), 7.92-7.89 (1H, m, |
| | ArH), 7.76-7.72 (1H, m, ArH), 7.29 (1H, s, ArH), 7.25 (1H, s, ArH), 4.03 |
| | (2H, s, NCH₂), 3.35-3.23 (6H, m, CH₂), 2.92 (4H, br. s, CH₂), 2.46 (2H, d, |
| | CH₂), 2.01-1.93 (1H, m, CH), 1.26 (3H, t, CH₃), 0.95 (6H, d, CH₃). |
| A-173 | ¹H NMR δ (ppm) (MeOH-d4): 9.11 (1H, ~d, ArH), 7.83-7.80 (1H, m, |
| | ArH), 7.74-7.70 (1H, m, ArH), 6.82 (1H, s, ArH), 6.80 (1H, d, ArH), 4.42 |
| | (1H, d, NCH₂), 3.96 (1H, d, NCH₂), 2.98-2.58 (7H, m, CH, CH₂), 2.53 |
| | (2H, d, CH₂), 1.94-1.86 (1H, m, CH), 1.06 (3H, d, CH₃), 0.93 (6H, d, |
| | CH₃). |
| A-174 | ¹H NMR δ (ppm) (MeOH-d4): 9.15 (1H, ~d, ArH), 7.86-7.83 (1H, m, |
| | ArH), 7.78-7.73 (1H, m, ArH), 6.85 (1H, s, ArH), 6.82 (1H, d, ArH), 4.44 |
| | (1H, d, NCH₂), 3.98 (1H, d, NCH₂), 2.99-2.58 (7H, m, CH, CH₂), 2.57 |
| | (2H, d, CH₂), 1.97-1.93 (1H, m, CH), 1.09 (3H, d, CH₃), 0.96 (6H, d, |
| | CH₃). |
| A-175 | ¹H NMR δ (ppm) (MeOH-d4): 9.14 (1H, ~d, ArH), 7.86-7.82 (1H, m, |
| | ArH), 7.77-7.73 (1H, m, ArH), 6.85 (1H, s, ArH), 6.82 (1H, d, ArH), 4.43 |
| | (1H, d, NCH₂), 3.96 (1H, d, NCH₂), 2.96-2.58 (7H, m, CH, CH₂), 2.57 |
| | (2H, d, CH₂), 1.97-1.93 (1H, m, CH), 1.08 (3H, d, CH₃), 0.97 (6H, d, |
| | CH₃). |
| A-176 | ¹H NMR δ (ppm) (MeOH-d4): 9.16 (1H, ~d, ArH), 7.89-7.85 (1H, m, |
| | ArH), 7.80-7.74 (1H, m, ArH), 7.30 (1H, d, ArH), 7.17 (1H, ~t, ArH), |
| | 4.54 (1H, d, NCH₂), 4.08 (1H, d, NCH₂), 3.21-2.96 (6H, m, CH, CH₂), |
| | 2.78-2.68 (1H, m, CH), 2.60 (2H, d, CH₂), 1.97-1.93 (1H, m, CH), 1.17 |
| | (3H, d, CH₃), 0.96 (6H, d, CH₃). |
| A-177 | ¹H NMR δ (ppm) (MeOH-d4): 9.10 (1H, ~d, ArH), 7.87 (1H, ~d, ArH), |
| | 7.76-7.70 (1H, m, ArH), 7.49 (2H, s, ArH), 4.42 (1H, d, NCH₂), 3.90 (1H, |
| | d, NCH₂), 3.18-2.42 (9H, m, CH, CH₂), 1.90-1.78 (1H, m, CH), 1.17 (3H, |
| | br. s, CH₃), 0.88 (6H, br. s, CH₃). |
| A-178 | ¹H NMR δ (ppm) (MeOH-d4): 9.11 (1H, ~d, ArH), 7.83-7.80 (1H, m, |
| | ArH), 7.74-7.70 (1H, m, ArH), 6.80 (1H, s, ArH), 6.77 (1H, d, ArH), 4.43 |
| | (1H, ~d, NCH₂), 3.93 (1H, ~d, NCH₂), 3.03-2.86 (4H, m, CH, CH₂), 2.68- |
| | 2.56 (2H, m, CH₂), 2.53 (2H, d, CH₂), 2.48-2.42 (1H, m, CH₂), 1.95-1.88 |
| | (1H, m, CH), 1.74-1.66 (1H, m, CH₂), 1.38-1.28 (1H, m, CH₂), 0.93 (6H, |
| | d, CH₃), 0.69 (3H, t, CH₃). |
| A-179 | ¹H NMR δ (ppm) (MeOH-d4): 9.10 (1H, ~d, ArH), 7.86-7.81 (1H, m, |
| | ArH), 7.74-7.70 (1H, m, ArH), 6.80 (1H, s, ArH), 6.78 (1H, d, ArH), 4.44 |
| | (1H, ~d, NCH₂), 3.77 (1H, ~d, NCH₂), 3.02-2.91 (2H, m, CH, CH₂), 2.82 |
| | (2H, ~t, CH₂), 2.65-2.50 (2H, m, CH₂), 2.51 (2H, d, CH₂), 2.28-2.14 (2H, |
| | m, CH, CH₂), 1.95-1.88 (1H, m, CH), 0.93 (6H, d, CH₃), 0.77 (3H, d, |
| | CH₃), 0.69 (3H, d, CH₃). |
| A-180 | ¹H NMR δ (ppm) (MeOH-d4): 9.12 (1H, ~d, ArH), 7.83-7.80 (1H, m, |
| | ArH), 7.75-7.71 (1H, m, ArH), 7.02 (1H, s, ArH), 6.93 (1H, d, ArH), 4.06 |
| | (2H, s, NCH₂), 3.16 (2H, br. s, CH₂), 2.96-2.87 (3H, m, CH, CH₂), 2.59- |
| | 2.45 (1H, m, CH₂), 2.57 (2H, d, CH₂), 2.29 (1H, ~t, CH₂), 1.96-1.87 (1H, |
| | m, CH), 0.93 (6H, d, CH₃), 0.85 (3H, d, CH₃). |
| A-181 | ¹H NMR δ (ppm) (MeOH-d4): 9.11 (1H, ~d, ArH), 7.84-7.81 (1H, m, |
| | ArH), 7.75-7.71 (1H, m, ArH), 6.93 (1H, s, ArH), 6.84 (1H, d, ArH), 4.00 |
| | (2H, dd, NCH₂), 2.95 (1H, ~t, CH), 2.78 (2H, br. s, CH₂), 2.69-2.60 (1H, |
| | m, CH₂), 2.67 (2H, s, CH₂), 2.55 (2H, d, CH₂), 2.41-2.35 (1H, m, CH₂), |
| | 1.95-1.88 (1H, m, CH), 1.48-1.24 (2H, m, CH₂), 0.93 (6H, d, CH₃), 0.60 |
| | (3H, t, CH₃). |
| A-182 | ¹H NMR δ (ppm) (CHCl3-d): 9.05 (1H, ~d, ArH), 7.74 (1H, ~d, ArH), |
| | 7.50 (1H, ~t, ArH), 6.75 (1H, d, ArH), 6.73 (1H, s, ArH), 3.83 (2H, s, |
| | NCH₂), 2.79 (2H, br. s, CH₂), 2.62 (2H, br. s, CH₂), 2.55-2.42 (2H, m, |
| | CH₂), 2.47 (2H, d, CH₂), 1.90-1.83 (1H, m, CH), 1.00 (6H, s, CH₃), 0.90 |
| | (6H, d, CH₃). |
| A-183 | ¹H NMR δ (ppm) (MeOH-d4): 9.26 (1H, ~d, ArH), 7.81 (2H, s, ArH), |
| | 7.17 (1H, s, ArH), 7.10 (1H, d, ArH), 5.05 (1H, ~d, NCH₂), 4.53 (1H, ~d, |
| | NCH₂), 3.52-3.42 (4H, m, CH₂), 3.08 (1H, ~t, CH), 2.80 (1H, ~t, CH), |
| | 2.62 (2H, d, CH₂), 1.97-1.91 (1H, m, CH), 1.48 (3H, d, CH₃), 0.95 (6H, d, |
| | CH₃), 0.78 (3H, d, CH₃). |
| A-184 | ¹H NMR δ (ppm) (MeOH-d4): 9.19 (1H, ~d, ArH), 7.88-7.84 (1H, m, |
| | ArH), 7.80-7.75 (1H, m, ArH), 6.91 (1H, s, ArH), 6.88 (1H, d, ArH), 4.65 |
| | (2H, s, NCH₂), 3.38-3.26 (2H, m, CH₂), 3.12-3.08 (2H, m, CH), 2.93 |
| | (2H, ~t, CH₂), 2.56 (2H, d, CH₂), 1.96-1.91 (1H, m, CH), 1.18 (6H, d, CH₃), |
| | 0.95 (6H, d, CH₃). |
| A-185 | ¹H NMR δ (ppm) (MeOH-d4): 9.00 (1H, ~d, ArH), 7.90 (1H, ~d, ArH), |
| | 7.67-7.64 (1H, m, ArH), 6.69 (1H, s, ArH), 6.63 (1H, d, ArH), 4.07 (1H, |
| | d, NCH₂), 3.79 (1H, d, NCH₂), 2.86 (2H, ~d, CH, CH₂), 2.77-2.67 (4H, m, |
| | CH, CH₂), 2.49 (2H, d, CH₂), 1.91-1.88 (1H, m, CH), 0.93 (6H, d, CH₃), |
| | 0.77 (6H, d, CH₃). |
| A-186 | ¹H NMR δ (ppm) (MeOH-d4): 9.11 (1H, ~d, ArH), 7.89 (1H, ~d, ArH), |
| | 7.75-7.72 (1H, m, ArH), 6.80 (2H, br. s, ArH), 4.35 (2H, s, NCH₂), 2.93 |
| | (4H, br. s, CH₂), 2.68 (2H, br. s, CH), 2.55 (2H, d, CH₂), 1.96-1.91 (1H, |
| | m, CH), 1.02 (6H, br. s, CH₃), 0.92 (6H, d, CH₃). |
| A-187 | ¹H NMR δ (ppm) (MeOH-d4): 9.00 (1H, ~d, ArH), 7.91 (1H, ~d, ArH), |
| | 7.67-7.63 (1H, m, ArH), 6.69 (1H, s, ArH), 6.63 (1H, d, ArH), 4.07 (1H, |
| | d, NCH₂), 3.79 (1H, d, NCH₂), 2.86 (2H, ~d, CH, CH₂), 2.76-2.66 (4H, m, |
| | CH, CH₂), 2.50 (2H, d, CH₂), 1.89-1.87 (1H, m, CH), 0.93 (6H, d, CH₃), |
| | 0.77 (6H, d, CH₃). |
| A-188 | ¹H NMR δ (ppm) (MeOH-d4): 9.11 (1H, ~d, ArH), 7.88-7.84 (1H, m, |
| | ArH), 7.75-7.71 (1H, m, ArH), 6.91-6.84 (2H, m, ArH), 4.29 (1H, ~d, |
| | NCH₂), 3.96 (1H, br. s, NCH₂), 3.32-3.22 (1H, m, CH₂), 3.02-2.62 (5H, |
| | m, CH, CH₂), 2.55 (2H, d, CH₂), 1.96-1.87 (1H, m, CH), 0.93 (6H, d, |
| | CH₃), 0.84 (3H, d, CH₃), 0.82 (3H, d, CH₃). |
| A-189 | ¹H NMR δ (ppm) (MeOH-d4): 9.15 (1H, s, ArH), 7.75 (2H, s, ArH), 6.46 |
| | (1H, s, ArH), 6.44 (1H, d, ArH), 4.50 (2H, dd, NCH₂), 4.07 (2H, br. s, |
| | CH₂), 3.48-3.41 (1H, m, CH₂), 3.21-3.17 (1H, m, CH₂), 3.05-3.01 (1H, m, |
| | CH), 2.88-2.84 (1H, m, CH), 2.47 (2H, d, CH₂), 2.16-2.11 (1H, m, CH₂), |
| | 2.05-2.01 (1H, m, CH₂), 1.92-1.86 (1H, m, CH), 0.92 (6H, d, CH₃). |
| A-190 | ¹H NMR δ (ppm) (MeOH-d4): 9.07 (1H, ~d, ArH), 7.91 (1H, ~d, ArH), |
| | 7.73-7.68 (1H, m, ArH), 6.73 (1H, s, ArH), 6.65 (1H, d, ArH), 3.95 (2H, |
| | s, NCH₂), 3.22 (2H, br. s, CH₂), 2.98 (2H, ~d, CH, CH₂), 2.79 (2H, ~d, |
| | CH, CH₂), 2.48 (2H, d, CH₂), 1.90-1.82 (3H, m, CH, CH₂), 1.52-1.48 (2H, |
| | m, CH₂), 0.91 (6H, d, CH₃). |
| A-191 | ¹H NMR δ (ppm) (MeOH-d4): 8.52 (1H, d, ArH), 7.57 (1H, s, ArH), 7.54- |
| | 7.44 (1H, m, ArH), 6.81-6.78 (2H, m, ArH), 4.07 (2H, s, CH₂), 2.99 (4H, |
| | br. s., CH₂), 2.86 (4H, br. s, CH₂), 2.55 (2H, d, CH₂), 1.94-1.91 (1H, m, |
| | CH), 0.94 (6H, d, CH₃). |
| A-192 | ¹H NMR δ (ppm) (MeOH-d4): 8.61 (1H, s, ArH), 7.93-7.89 (1H, m, |
| | ArH), 7.49 (1H, d, ArH), 6.81-6.78 (2H, m, ArH), 4.16 (2H, s, CH₂), 3.01 |
| | (4H, br. s., CH₂), 2.94 (4H, br. s., CH₂), 2.55 (2H, d, CH₂), 1.94-1.91 (1H, |
| | m, CH), 0.96 (6H, d, CH₃). |
| A-193 | ¹H NMR δ (ppm) (MeOH-d4): 8.55 (1H, d, ArH), 7.93 (1H, d, ArH), |
| | 7.43-7.40 (1H, m, ArH), 6.81-6.78 (2H, m, ArH), 4.42 (2H, s, CH₂), 3.11- |
| | 3.05 (8H, m, CH₂), 2.55 (2H, d, CH₂), 1.91-1.87 (1H, m, CH), 0.95 (6H, |
| | d, CH₃). |
| A-194 | ¹H NMR δ (ppm) (MeOH-d4): 8.30 (1H, s, ArH), 7.41 (2H, s, ArH), 6.76 |
| | (2H, br. s, ArH), 4.18 (2H, s, CH₂), 3.88 (3H, s, OCH₃), 3.01 (8H, br. s, |
| | CH₂), 2.52 (2H, d, CH₂), 1.93-1.89 (1H, m, CH), 0.93 (6H, d, CH₃). |
| A-195 | ¹H NMR δ (ppm) (MeOH-d4): 8.50 (1H, s, ArH), 7.65-7.56 (1H, m, |
| | ArH), 7.53-7.49 (1H, m, ArH), 6.78 (2H, br. s, ArH), 4.18 (2H, s, CH₂), |
| | 3.00 (4H, br. s, CH₂), 2.94 (4H, br. s, CH₂), 2.52 (2H, d, CH₂), 1.92-1.87 |
| | (1H, m, CH), 0.92 (6H, d, CH₃). |
| A-196 | ¹H NMR δ (ppm) (MeOH-d4): 8.45 (1H, d, ArH), 7.67 (1H, t, ArH), 7.50- |
| | 7.48 (1H, m, ArH), 6.79 (2H, br. s, ArH), 4.24 (2H, s, CH₂), 3.00 (8H, br. |
| | s, CH₂), 2.53 (2H, d, CH₂), 1.94-1.92 (1H, m, CH), 0.92 (6H, d, CH₃). |
| A-197 | ¹H NMR δ (ppm) (MeOH-d4): 8.26 (1H, s, ArH), 7.50 (1H, s, ArH), 6.79- |
| | 6.75 (2H, m, ArH), 4.31 (2H, s, CH₂), 3.13 (4H, br. s, CH₂), 3.09 (4H, br. |
| | s, CH₂), 2.52 (2H, d, CH₂), 2.31 (3H, s, CH₃), 2.28 (3H, s, CH₃), 1.93- |
| | 1.91 (1H, m, CH), 0.94 (6H, d, CH₃). |
| A-198 | ¹H NMR δ (ppm) (MeOH-d4): 8.18 (1H, d, ArH), 7.50 (1H, d, ArH), |
| | 7.40-7.39 (1H, m, ArH), 6.77-6.76 (2H, m, ArH), 4.36 (2H, s, CH₂), 3.91 |
| | (3H, s, OCH₃), 3.16 (4H, br. s, CH₂), 3.08 (4H, br. s, CH₂), 2.53 (2H, d, |
| | CH₂), 1.93-1.90 (1H, m, CH), 0.93 (6H, d, CH₃). |
| A-199 | ¹H NMR δ (ppm) (MeOH-d4): 8.39 (1H, d, ArH), 7.10 (1H, s, ArH), 7.01- |
| | 6.99 (1H, m, ArH), 6.79-6.75 (2H, m, ArH), 4.05 (2H, s, CH₂), 3.92 (3H, |
| | s, OCH₃), 2.99 (4H, br. s, CH₂), 2.86 (4H, br. s, CH₂), 2.54 (2H, d, CH₂), |
| | 1.93-1.91 (1H, m, CH), 0.93 (6H, d, CH₃). |
| A-200 | ¹H NMR δ (ppm) (MeOH-d4): 8.23 (1H, s, ArH), 6.77 (2H, br. s, ArH), |
| | 4.25 (2H, s, CH₂), 3.80 (3H, s, OCH₃), 3.06 (8H, br. s, CH₂), 2.52 (2H, d, |
| | CH₂), 2.26 (3H, s, CH₃), 2.21 (3H, s, CH₃), 1.92-1.89 (1H, m, CH), 0.93 |
| | (6H, d, CH₃). |
| A-201 | ¹H NMR δ (ppm) (MeOH-d4): 8.45 (1H, s, ArH), 7.68 (1H, d, ArH), 7.35 |
| | (1H, d, ArH), 6.77 (2H, br. s, ArH), 4.18 (2H, s, CH₂), 3.01 (8H, br. s, |
| | CH₂), 2.52 (2H, d, CH₂), 2.35 (3H, s, CH₃), 1.93-1.89 (1H, m, CH), 0.92 |
| | (6H, d, CH₃). |
| A-202 | ¹H NMR δ (ppm) (MeOH-d4): 8.82 (1H, d, ArH), 7.79 (1H, s, ArH), 7.67 |
| | (1H, m, ArH), 6.80 (2H, br. s, ArH), 4.15 (2H, s, CH₂), 2.97 (4H, br. s, |
| | CH₂), 2.84 (4H, br. s, CH₂), 2.55 (2H, d, CH₂), 1.93-1.91 (1H, m, CH), |
| | 0.93 (6H, d, CH₃). |
| A-203 | ¹H NMR δ (ppm) (DMSO-d6): 8.31-8.29 (1H, m, ArH), 7.56 (1H, d, |
| | ArH), 7.22-7.19 (1H, m, ArH), 6.87-6.80 (2H, m, ArH), 3.67 (2H, s, |
| | CH₂), 2.74 (4H, br. s, CH₂), 2.50 (2H, br. s, CH₂), 2.38 (4H, br. s, CH₂), |
| | 2.33 (3H, s, CH₃), 1.91-1.87 (1H, m, CH), 0.88 (6H, d, CH₃). |
| A-204 | ¹H NMR δ (ppm) (DMSO-d6): 8.33-8.31 (1H, m, ArH), 7.21 (1H, s, |
| | ArH), 7.09-7.08 (1H, m, ArH), 6.86-6.78 (2H, m, ArH), 3.58 (2H, s, |
| | CH₂), 2.75 (4H, br. s, CH₂), 2.50 (2H, br. s, CH₂), 2.37 (4H, br. s, CH₂), |
| | 2.28 (3H, s, CH₃), 1.91-1.84 (1H, m, CH), 0.88 (6H, d, CH₃). |
| A-205 | ¹H NMR δ (ppm) (MeOH-d4): 8.49 (1H, s, ArH), 7.88 (1H, d, ArH), 6.82- |
| | 6.78 (2H, m, ArH), 4.14 (2H, s, CH₂), 2.98 (4H, br. s, CH₂), 2.90 (4H, br. |
| | s, CH₂), 2.56 (2H, d, CH₂), 1.96-1.93 (1H, m, CH), 0.97 (6H, d, CH₃). |
| A-206 | ¹H NMR δ (ppm) (MeOH-d4): 8.50 (1H, s, ArH), 7.88 (1H, d, ArH), 6.81 |
| | (2H, br. s, ArH), 4.14 (2H, s, CH₂), 2.97 (4H, br. s, CH₂), 2.90 (4H, br. s, |
| | CH₂), 2.55 (2H, d, CH₂), 1.96-1.92 (1H, m, CH), 0.96 (6H, d, CH₃). |
| A-207 | ¹H NMR δ (ppm) (DMSO-d6): 8.04 (1H, s, ArH), 7.00 (1H, s, ArH), 6.81 |
| | (2H, br. s, ArH), 3.96 (1H, s, NH), 3.81 (6H, s, OCH₃), 3.54 (2H, s, CH₂), |
| | 2.77 (4H, br. s, CH₂), 2.50 (2H, br. s, CH₂), 2.37 (4H, br. s, CH₂), 1.91- |
| | 1.87 (1H, m, CH), 0.89 (6H, d, CH₃). |
| A-208 | ¹H NMR δ (ppm) (MeOH-d4): 8.35 (1H, s, ArH), 7.12 (1H, s, ArH), 6.76 |
| | (2H, br. s, ArH), 4.17 (2H, s, CH₂), 3.98 (3H, s, OCH₃), 3.02 (4H, br. s, |
| | CH₂), 2.93 (4H, br. s, CH₂), 2.51 (2H, d, CH₂), 2.17 (3H, s, CH₃), 1.93- |
| | 1.91 (1H, m, CH), 0.92 (6H, d, CH₃). |
| A-209 | ¹H NMR δ (ppm) (MeOH-d4): 8.91 (1H, s, ArH), 8.19 (1H, d, ArH), 7.67 |
| | (1H, d, ArH), 6.82 (2H, br. s, ArH), 4.07 (2H, s, CH₂), 2.97 (4H, br. s, |
| | CH₂), 2.78 (4H, br. s, CH₂), 2.55 (2H, d, CH₂), 1.95-1.92 (1H, m, CH), |
| | 0.95 (6H, d, CH₃). |
| A-210 | ¹H NMR δ (ppm) (MeOH-d4): 8.56 (1H, s, ArH), 7.70 (1H, d, ArH), 6.78 |
| | (2H, br. s, ArH), 4.02 (2H, s, CH₂), 2.94 (4H, br. s, CH₂), 2.88 (4H, br. s, |
| | CH₂), 2.54 (2H, d, CH₂), 1.94-1.92 (1H, m, CH), 0.95 (6H, d, CH₃). |
| A-211 | ¹H NMR δ (ppm) (MeOH-d4): 8.44 (1H, s, ArH), 7.68 (1H, t, ArH), 6.78 |
| | (2H, br. s, ArH), 4.13 (2H, s, CH₂), 2.96 (4H, br. s, CH₂), 2.89 (4H, br. s, |
| | CH₂), 2.54 (2H, d, CH₂), 1.94-1.91 (1H, m, CH), 0.95 (6H, d, CH₃). |
| A-212 | ¹H NMR δ (ppm) (MeOH-d4): 9.11 (1H, d, ArH), 7.84 (1H, d, ArH), |
| | 7.75-7.72 (1H, m, ArH), 7.59 (1H, d, ArH), 7.06 (1H, s, ArH), 7.02 (1H, |
| | d, ArH), 4.07 (1H, q, CH), 2.91-2.84 (6H, m, CH₂), 2.65-2.63 (2H, m, |
| | CH₂), 2.53 (2H, d, CH₂), 1.91-1.88 (1H, m, CH), 1.51 (3H, d, CH₃), 0.92 |
| | (6H, d, CH₃). |
| A-213 | ¹H NMR δ (ppm) (MeOH-d4): 9.23 (1H, ~d, ArH), 7.90-7.80 (2H, m, |
| | ArH), 6.89 (1H, s, ArH), 6.84 (1H, d, ArH), 3.74 (2H, ~t, NCH₂), 3.73 |
| | (2H, ~t, NCH₂), 2.97 (2H, ~t, CH₂), 2.86 (2H, ~t, CH₂), 2.55 (2H, d, |
| | CH₂), 1.96-1.90 (1H, m, CH), 0.93 (6H, d, CH₃). |
| A-214 | ¹H NMR δ (ppm) (MeOH-d4): 8.63 (1H, ~d, ArH), 7.86 (1H, ~t, ArH), |
| | 7.46-7.38 (2H, m, ArH), 6.76 (1H, d, ArH), 6.75 (1H, s, ArH), 4.35 (1H, |
| | ~q, NCH), 3.12 (2H, br. s, CH₂), 3.03 (4H, ~t, CH₂), 2.91-2.84 (2H, m, |
| | CH₂), 2.52 (2H, d, CH₂), 1.93-1.89 (1H, m, CH), 1.58 (3H, d, CH₃), 0.93 |
| | (6H, d, CH₃). |
| A-215 | ¹H NMR δ (ppm) (MeOH-d4): 8.56 (1H, ~d, ArH), 7.93 (1H, ~t, ArH), |
| | 7.58 (1H, ~d, ArH), 7.48 (1H, ~t, ArH), 6.90 (1H, s, ArH), 6.86 (1H, d, |
| | ArH), 3.71 (2H, br. s, CH₂), 3.37 (2H, br. s, CH₂), 2.95 (2H, br. s, CH₂), |
| | 2.81 (2H, br. s, CH₂), 2.56 (2H, d, CH₂), 1.98-1.91 (1H, m, CH), 0.93 |
| | (6H, d, CH₃). |
| A-216 | ¹H NMR δ (ppm) (MeOH-d4): 8.57 (1H, ~d, ArH), 7.95 (1H, ~t, ArH), |
| | 7.55 (1H, ~d, ArH), 7.49 (1H, ~t, ArH), 6.95 (1H, s, ArH), 6.89 (1H, d, |
| | ArH), 4.71-4.42 (1H, m, CH₂), 3.80-3.46 (1H, m, CH₂), 3.21-2.64 (5H, m, |
| | CH, CH₂), 2.58 (2H, d, CH₂), 1.98-1.91 (1H, m, CH), 0.95 (9H, d, CH₃). |
| A-217 | ¹H NMR δ (ppm) (MeOH-d4): 8.61 (1H, ~d, ArH), 7.86 (1H, ~t, ArH), |
| | 7.48 (1H, ~d, ArH), 7.40 (1H, ~t, ArH), 6.78 (1H, s, ArH), 6.77 (1H, d, |
| | ArH), 4.51 (1H, d, NCH₂), 4.14 (1H, d, NCH₂), 3.21-2.84 (7H, m, CH, |
| | CH₂), 2.53 (2H, d, CH₂), 1.94-1.90 (1H, m, CH), 1.14 (3H, d, CH₃), 0.93 |
| | (6H, d, CH₃). |
| A-218 | ¹H NMR δ (ppm) (MeOH-d4): 8.63 (1H, ~t, ArH), 7.86 (1H, ~t, ArH), |
| | 7.48 (1H, ~t, ArH), 7.39 (1H, br. s, ArH), 6.76 (2H, s, ArH), 4.60 (1H, br. |
| | s, NCH), 3.21-2.81 (7H, m, CH, CH₂), 2.52 (2H, d, CH₂), 1.93-1.89 (1H, |
| | m, CH), 1.59-1.51 (3H, m, CH₃), 0.93 (6H, d, CH₃). |
| A-219 | ¹H NMR δ (ppm) (MeOH-d4): 7.40 (1H, s, ArH), 7.34 (1H, ~d, ArH), |
| | 7.16 (1H, ~t, ArH), 3.97 (2H, s, NCH₂), 3.13 (4H, ~t, CH₂), 2.69 (4H, br. |
| | s, CH₂), 2.59 (2H, d, CH₂), 2.47 (3H, s, CH₃), 1.95-1.90 (1H, m, CH), |
| | 0.95 (6H, d, CH₃). |
| A-220 | ¹H NMR δ (ppm) (MeOH-d4): 7.37 (1H, s, ArH), 6.83 (2H, br. s, ArH), |
| | 3.89 (2H, s, NCH₂), 2.86 (4H, br. s, CH₂), 2.60 (4H, br. s, CH₂), 2.53 (2H, |
| | d, CH₂), 2.44 (3H, s, CH₃), 1.94-1.89 (1H, m, CH), 0.93 (6H, d, CH₃). |
| A-221 | ¹H NMR δ (ppm) (MeOH-d4): 7.85 (1H, br. s, ArH), 7.58 (1H, br. s, |
| | ArH), 7.45 (1H, s, ArH), 4.13 (2H, s, NCH₂), 3.17 (4H, br. s, CH₂), 3.07 |
| | (4H, br. s, CH₂), 2.50 (5H, br. s, CH₃, CH₂), 1.96-1.82 (1H, m, CH), 0.88 |
| | (6H, d, CH₃). |
| A-222 | ¹H NMR δ (ppm) (MeOH-d4): 7.40 (1H, s, ArH), 6.83 (2H, br. s, ArH), |
| | 4.21 (1H, d, NCH₂), 3.90 (1H, d, NCH₂), 2.89 (4H, br. s, CH, CH₂), 2.76- |
| | 2.45 (3H, m, CH₂), 2.54 (2H, d, CH₂), 2.46 (3H, s, CH₃), 1.94-1.90 (1H, |
| | m, CH), 1.02 (3H, br. s, CH₃), 0.94 (6H, d, CH₃). |
| A-223 | ¹H NMR δ (ppm) (MeOH-d4): 7.40 (1H, s, ArH), 6.83 (2H, br. s, ArH), |
| | 4.21 (1H, d, NCH₂), 3.90 (1H, d, NCH₂), 2.89 (4H, br. s, CH, CH₂), 2.72- |
| | 2.44 (3H, m, CH₂), 2.54 (2H, d, CH₂), 2.46 (3H, s, CH₃), 1.94-1.90 (1H, |
| | m, CH), 1.02 (3H, br. s, CH₃), 0.94 (6H, d, CH₃). |
| A-224 | ¹H NMR δ (ppm) (MeOH-d4): 7.44 (1H, s, ArH), 6.81 (2H, br. s, ArH), |
| | 4.25 (2H, s, NCH₂), 2.96-2.77 (4H, m, CH, CH₂), 2.68-2.52 (2H, m, CH₂), |
| | 2.55 (2H, d, CH₂), 2.48 (3H, s, CH₃), 1.96-1.93 (1H, m, CH), 1.11 (6H, |
| | br. s, CH₃), 0.95 (6H, d, CH₃). |
| A-225 | ¹H NMR δ (ppm) (MeOH-d4): 7.38 (1H, s, ArH), 6.83 (1H, s, ArH), 6.79 |
| | (1H, ~d, ArH), 4.08 (1H, ~q, NCH), 2.88 (4H, ~t, CH₂), 2.68-2.58 (4H, m, |
| | CH₂), 2.54 (2H, d, CH₂), 2.45 (3H, s, CH₃), 1.95-1.90 (1H, m, CH), 1.47 |
| | (3H, d, CH₃), 0.94 (6H, d, CH₃). |
| A-226 | ¹H NMR δ (ppm) (MeOH-d4): 7.61 (1H, s, ArH), 6.94 (2H, br. s, ArH), |
| | 3.28-2.99 (4H, m, CH₂, CH), 2.97-2.69 (4H, m, CH₂), 2.67 (2H, d, CH₂), |
| | 2.63 (3H, s, CH₃), 2.07-2.03 (1H, m, CH), 1.71 (3H, d, CH₃), 1.23 (3H, |
| | br. s, CH₃), 1.07 (6H, d, CH₃). |
| A-227 | ¹H NMR δ (ppm) (MeOH-d4): 8.43 (1H, d, ArH), 7.13 (1H, s, ArH), 7.04- |
| | 7.01 (1H, m, ArH), 6.79 (1H, s, ArH), 6.78 (1H, d, ArH), 4.41 (1H, ~d, |
| | NCH₂), 4.00 (1H, ~d, NCH₂), 3.94 (3H, s, OCH₃), 3.09-3.02 (5H, m, CH, |
| | CH₂), 2.85 (2H, ~t, CH₂), 2.55 (2H, d, CH₂), 1.96-1.92 (1H, m, CH), 1.10 |
| | (3H, d, CH₃), 0.96 (6H, d, CH₃). |
| A-228 | ¹H NMR δ (ppm) (MeOH-d4): 8.45 (1H, d, ArH), 7.70 (1H, ~d, ArH), |
| | 7.35-7.31 (1H, m, ArH), 6.82 (1H, s, ArH), 6.80 (1H, d, ArH), 4.60 (1H, ~d, |
| | NCH₂), 4.30 (1H, ~d, NCH₂), 3.41-3.23 (2H, m, CH, CH₂), 3.22-2.98 |
| | (5H, m, CH₂), 2.56 (2H, d, CH₂), 2.37 (3H, s, CH₃), 1.97-1.92 (1H, m, |
| | CH), 1.14 (3H, d, CH₃), 0.97 (6H, d, CH₃). |
| A-229 | ¹H NMR δ (ppm) (MeOH-d4): 8.45 (1H, d, ArH), 7.33 (1H, s, ArH), 7.25 |
| | (1H, d, ArH), 6.78 (1H, s, ArH), 6.77 (1H, d, ArH), 4.46 (1H, ~d, NCH₂), |
| | 4.08 (1H, ~d, NCH₂), 3.18-2.99 (5H, m, CH, CH₂), 2.94-2.86 (2H, m, |
| | CH₂), 2.53 (2H, d, CH₂), 2.40 (3H, s, CH₃), 1.94-1.90 (1H, m, CH), 1.13 |
| | (3H, d, CH₃), 0.95 (6H, d, CH₃). |
| A-230 | ¹H NMR δ (ppm) (MeOH-d4): 9.17 (1H, ~d, ArH), 8.18 (1H, ~d, ArH), |
| | 7.91 (1H, ~d, ArH), 7.80-7.75 (1H, m, ArH), 7.13 (1H, s, ArH), 7.05 (1H, ~d, |
| | ArH), 4.77 (2H, s, NCH₂), 3.42-3.22 (8H, m, CH₂), 2.51 (2H, d, CH₂), |
| | 2.11 (2H, ~t, CH₂), 1.92-1.86 (1H, m, CH), 0.92 (6H, d, CH₃). |
| A-231 | ¹H NMR δ (ppm) (MeOH-d4): 9.21 (1H, ~d, ArH), 7.96 (1H, ~d, ArH), |
| | 7.83-7.80 (1H, m, ArH), 6.86 (1H, s, ArH), 6.76 (1H, ~d, ArH), 4.62 (2H, |
| | s, NCH₂), 3.38-3.22 (4H, m, CH₂), 3.20 (2H, ~t, CH₂), 2.98 (2H, ~t, CH₂), |
| | 2.52 (2H, d, CH₂), 1.96-1.91 (3H, m, CH, CH₂), 0.93 (6H, d, CH₃). |
| A-232 | ¹H NMR δ (ppm) (MeOH-d4): 9.12 (1H, ~d, ArH), 8.03 (1H, ~d, ArH), |
| | 7.85 (1H, ~d, ArH), 7.73-7.68 (1H, m, ArH), 6.95 (1H, ~d, ArH), 6.88 |
| | (1H, s, ArH), 3.61-3.56 (2H, m, NCH₂), 3.41 (2H, ~t, CH₂), 3.11-3.02 |
| | (6H, m, CH, CH₂), 2.90 (2H, br. s, CH₂), 2.48 (2H, d, CH₂), 1.90-1.86 |
| | (1H, m, CH), 0.91 (6H, d, CH₃). |
| A-233 | ¹H NMR δ (ppm) (MeOH-d4): 9.92 (1H, ~d, ArH), 8.04 (1H, ~d, ArH), |
| | 7.80-7.76 (1H, m, ArH), 6.70 (1H, ~d, ArH), 6.63 (1H, s, ArH), 4.82 (2H, |
| | s, NCH₂), 3.54 (2H, br. s, CH₂), 3.20 (2H, br. s, CH₂), 2.99 (2H, br. s, |
| | CH₂), 2.82-2.73 (4H, m, CH, CH₂), 2.49 (2H, d, CH₂), 1.92-1.87 (1H, m, |
| | CH), 0.92 (6H, d, CH₃). |
| A-234 | ¹H NMR δ (ppm) (MeOH-d4): 9.19 (1H, ~d, ArH), 7.84-7.75 (2H, m, |
| | ArH), 7.38-7.32 (1H, m, ArH), 7.16 (1H, s, ArH), 7.01 (1H, d, ArH), 4.39 |
| | (2H, s, NCH₂), 3.36 (2H, d, CH₂), 3.23 (1H, br. s, CH), 2.80 (2H, br. s, |
| | CH₂), 2.47 (2H, d, CH₂), 1.93 (4H, br. s, CH₂), 1.98-1.82 (1H, m, CH), |
| | 0.88 (6H, d, CH₃). |
| A-235 | ¹H NMR δ (ppm) (MeOH-d4): 9.21 (1H, ~d, ArH), 7.79 (2H, s, ArH), |
| | 7.52 (1H, ~d, ArH), 7.39 (1H, s, ArH), 7.18 (1H, ~d, ArH), 4.73 (2H, dd, |
| | NCH₂), 4.13-4.08 (1H, m, CH), 3.76-3.35 (4H, m, CH₂), 2.57 (2H, d, |
| | CH₂), 2.46-2.38 (1H, m, CH₂), 2.28-2.18 (1H, m, CH₂), 1.95-1.91 (1H, m, |
| | CH), 0.94 (6H, d, CH₃). |
| A-236 | ¹H NMR δ (ppm) (MeOH-d4): 7.77 (1H, ~d, ArH), 7.19 (1H, ~d, ArH), |
| | 6.96 (1H, ~d, ArH), 6.67 (2H, ~t, ArH), 6.55 (1H, ~d, ArH), 3.96 (1H, ~t, |
| | NCH), 3.56-3.25 (6H, m, CH₂), 2.43 (2H, d, CH₂), 1.89-1.84 (1H, m, |
| | CH), 0.91 (6H, d, CH₃). |
| A-237 | ¹H NMR δ (ppm) (MeOH-d4): 8.42 (1H, ~d, ArH), 7.75 (1H, t, ArH), |
| | 7.34-7.20 (2H, m, ArH), 6.84-6.68 (2H, m, ArH), 2.97 (1H, br. d, CH₂), |
| | 2.68 (2H, ~d, CH₂), 2.58 (2H, ~t, CH₂), 2.52 (2H, d, CH₂), 1.91 |
| | (1H, ~quint, CH), 1.85-1.70 (1H, m, CH), 1.49 (2H, br. d, CH₂), 1.25-1.15 (2H, |
| | m, CH₂), 0.93 (6H, d, CH₃). |
| A-238 | ¹H NMR δ (ppm) (MeOH-d4): 9.11 (1H, ~d, ArH), 8.03 (2H, ~d, ArH), |
| | 7.89-7.86 (1H, m, ArH), 7.76-7.72 (1H, m, ArH), 7.41 (1H, ~d, ArH), |
| | 7.38 (1H, s, ArH), 4.34 (2H, s, NCH₂), 4.00 (2H, s, NCH₂), 3.35 (4H, ~t, |
| | CH₂), 2.90 (4H, br. s, CH₂), 2.57 (2H, d, CH₂), 1.95-1.91 (1H, m, CH), |
| | 0.93 (6H, d, CH₃). |
| A-239 | ¹H NMR δ (ppm) (MeOH-d4): 9.05 (1H, ~d, ArH), 7.81 (2H, ~d, ArH), |
| | 7.71-7.65 (1H, m, ArH), 7.31 (1H, ~d, ArH), 7.15 (1H, s, ArH), 3.79 (3H, |
| | s, NCH₂, CH₂), 3.68-3.65 (1H, m, CH₂), 3.15-3.11 (1H, m, CH₂), 2.94- |
| | 2.89 (1H, m, CH₂), 2.61-2.45 (4H, m, CH₂), 2.20 (1H, ~t, CH₂), 1.95-1.87 |
| | (2H, m, CH, CH₂), 0.95 (3H, d, CH₃), 0.92 (3H, d, CH₃). |
| A-240 | ¹H NMR δ (ppm) (DMSO-d6): 8.08 (1H, d, ArH), 7.79-7.73 (1H, m, |
| | ArH), 7.65 (1H, ~d, ArH), 7.60-7.56 (1H, m, ArH), 7.50-7.44 (2H, m, |
| | ArH), 7.23-7.15 (2H, m, ArH), 4.03 (2H, ~d, NCH₂), 2.89 (2H, q, CH₂), |
| | 2.86-2.82 (2H, m, CH₂), 2.56-2.48 (2H, m, CH₂), 1.82-1.80 (1H, m, CH), |
| | 1.49 (4H, br. s, CH₂), 1.28 (3H, t, CH₃). |
| A-241 | ¹H NMR δ (ppm) (CHCl3-d): 15.60 (1H, s, NH), 8.27-8.23 (2H, m, ArH), |
| | 7.73 (1H, t, ArH), 7.65 (1H, d, ArH), 7.56 (1H, t, ArH), 7.44 (1H, t, ArH), |
| | 7.36 (1H, t, ArH), 7.30 (1H, d, ArH), 4.11 (2H, ~d, NCH₂), 3.56 (2H, q, |
| | CH₂), 3.17-3.11 (2H, m, CH₂), 2.93 (2H, q, CH₂), 2.87 (2H, ~t, CH₂), |
| | 2.31-2.27 (1H, m, CH), 1.87-1.80 (4H, m, CH₂), 1.45-1.37 (6H, m, CH₃). |
| A-242 | ¹H NMR δ (ppm) (DMSO-d6): 8.78 (1H, m, ArH), 8.50-8.47 (1H, m, |
| | ArH), 7.77 (1H, d, ArH), 7.68-7.63 (1H, m, ArH), 7.49-7.45 (1H, m, |
| | ArH), 7.21-7.13 (2H, m, ArH), 4.05 (2H, ~d, NCH₂), 2.95 (2H, q, CH₂), |
| | 2.85-2.82 (2H, m, CH₂), 2.55-2.52 (2H, m, CH₂), 1.84-1.81 (1H, m, CH), |
| | 1.50 (4H, br. s, CH₂), 1.29 (3H, t, CH₃). |
| A-243 | ¹H NMR δ (ppm) (DMSO-d6): 7.93 (1H, s, ArH), 7.67-7.59 (3H, m, |
| | ArH), 7.58 (1H, ~d, ArH), 7.52-7.44 (1H, m, ArH), 7.32-7.12 (2H, m, |
| | ArH), 6.82-6.80 (1H, m, ArH), 6.67-6.67 (1H, m, ArH), 4.00 (2H, s, |
| | NCH₂), 3.77 (2H, t, NCH₂), 2.91 (2H, q, CH₂), 2.88-2.82 (2H, m, CH₂), |
| | 2.55-2.52 (2H, m, CH₂), 1.82-1.78 (1H, m, CH), 1.54-1.47 (6H, m, CH₂), |
| | 1.29 (3H, t, CH₃), 0.84 (3H, t, CH₃). |
| A-244 | ¹H NMR δ (ppm) (MeOH-d4): 8.42-8.39 (2H, m, ArH), 7.90 (1H, s, |
| | ArH), 7.85-7.82 (2H, m, ArH), 7.61-7.60 (1H, m, ArH), 7.54-7.47 (3H, m, |
| | ArH), 7.70-7.37 (2H, m, ArH), 7.29-7.25 (1H, m, ArH), 6.93-6.92 (1H, m, |
| | ArH), 6.83-6.80 (1H, m, ArH), 5.18 (2H, s, NCH₂), 4.12 (2H, ~d, NCH₂), |
| | 3.11-3.08 (2H, m, CH₂), 2.94 (2H, q, CH₂), 2.84-2.79 (2H, m, CH₂), 2.02- |
| | 1.99 (1H, m, CH), 1.71-1.62 (4H, m, CH₂), 1.38 (3H, t, CH₃). |
| A-245 | ¹H NMR δ (ppm) (DMSO-d6): 10.51 (1H, s, ArH), 8.58 (1H, s, ArH), |
| | 8.16-8.13 (1H, m, ArH), 7.96 (1H, d, ArH), 7.65-7.44 (6H, m, ArH), 7.20- |
| | 7.12 (2H, m, ArH), 4.04 (2H, s, CH₂), 2.90-2.84 (4H, m, CH₂), 2.55-2.47 |
| | (2H, m, CH₂), 1.82-1.80 (1H, m, CH), 1.52-1.47 (4H, m, CH₂), 1.29 (3H, |
| | t, CH₃). |
| A-246 | ¹H NMR δ (ppm) (DMSO-d6): 7.75-7.72 (2H, m, ArH), 7.55-7.49 (4H, m, |
| | ArH), 7.40-7.37 (1H, m, ArH), 7.30-7.19 (6H, m, ArH), 6.82-6.80 (2H, m, |
| | ArH), 5.02 (2H, s, NCH₂), 3.99 (2H, s, NCH₂), 3.02-2.99 (2H, m, CH₂), |
| | 2.86-2.78 (4H, m, CH₂), 1.88-1.85 (1H, m, CH), 1.58-1.46 (4H, m, CH₂), |
| | 1.22 (3H, t, CH₃). |
| A-247 | ¹H NMR δ (ppm) (MeOH-d4): 8.39 (1H, s, ArH), 7.93 (1H, d, ArH), 7.86 |
| | (1H, d, ArH), 7.58 (1H, d, ArH), 7.53-7.49 (1H, m, ArH), 7.42 (1H, d, |
| | ArH), 7.29 (1H, t, ArH), 4.15 (2H, ~d, NCH₂), 3.14-3.11 (2H, m, CH₂), |
| | 2.93 (2H, q, CH₂), 2.87-2.82 (2H, m, CH₂), 2.04-2.02 (1H, m, CH), 1.80- |
| | 1.66 (5H, m, CH₂, CH), 1.38 (3H, t, CH₂), 0.98-0.94 (2H, m, CH₂), 0.91- |
| | 0.84 (2H, m, CH₂). |
| A-248 | ¹H NMR δ (ppm) (DMSO-d6): 7.89 (1H, s, ArH), 7.65-7.36 (7H, m, |
| | ArH), 7.31-7.14 (4H, m, ArH), 6.87-6.84 (1H, m, ArH), 6.77-6.46 (1H, m, |
| | ArH), 5.15 (2H, s, NCH₂), 3.97 (2H, s, NCH₂), 2.90-2.81 (4H, m, CH₂), |
| | 2.52-2.51 (2H, m, CH₂), 1.78-1.76 (1H, m, CH), 1.48-1.42 (4H, m, CH₂), |
| | 1.27 (3H, t, CH₃). |
| A-249 | ¹H NMR δ (ppm) (DMSO-d6): 8.17 (1H, d, ArH), 7.82 (1H, t, ArH), 7.65- |
| | 7.52 (8H, m, ArH), 7.44 (1H, t, ArH), 7.12 (2H, ~t, ArH), 3.95 (2H, ~d, |
| | NCH₂), 2.70-2.63 (2H, m, CH₂), 2.34 (2H, ~t, CH₂), 1.50 (1H, br. s, CH), |
| | 1.22 (2H, ~t, CH₂), 1.16-1.06 (2H, m, CH₂). |
| A-250 | ¹H NMR δ (ppm) (MeOH-d4): 9.13 (1H, ~d, ArH), 7.84-7.80 (1H, m, |
| | ArH), 7.76-7.70 (1H, m, ArH), 6.82 (1H, s, ArH), 6.80 (1H, d, ArH), 4.44 |
| | (1H, d, NCH2), 3.98 (1H, d, NCH2), 2.99-2.57 (7H, m, CH, CH2), 2.55- |
| | 2.50 (1H, m, CH), 1.07 (3H, d, CH3), 0.93 (6H, s, CH3). |
| A-251 | ¹H NMR δ (ppm) (MeOH-d4): 9.14 (1H, ~d, ArH), 7.83-7.80 (1H, m, |
| | ArH), 7.76-7.71 (1H, m, ArH), 6.81 (1H, s, ArH), 6.79 (1H, d, ArH), 4.48 |
| | (1H, d, NCH2), 4.04 (1H, d, NCH2), 2.54 (2H, d, CH2), 1.97-1.87 (1H, |
| | m, CH), 0.94 (6H, d, CH3). |

### Example 2: Binding Data

Table 3 below lists a number of representative compounds of the invention and their binding affinity for AT2R.

All synthesized ligands were evaluated in a radioligand assay by displacing ¹²⁵I-[Sar1, Ile8]-Angiotensin II (Perkin Elmer, NEX248050UC) from human AT2R fused to Cb23 or human/rodent/cynomolgus/minipig/dog WT AT2R in HEK-293 cells membrane preparations, using C21 (Vicore Pharma) and Angiotensin II (endogenous ligand) as reference. The affinity was determined using an eight-point dose-response curve, each point performed in duplicates. The compounds were also evaluated in a counterscreen binding assay for displacement of ¹²⁵I-[Sar1, Ile8]-Angiotensin II binding to human AT1R in HEK-293 cell membranes. For AT1R, the percent displacement was determined at 1 μM and 10 M (in duplicates) or using an eight-point dose-response curve, each point performed in duplicates with Candesartan and Losartan used as reference.

For the AT2R/AT1R binding assays, cell membranes, expressing AT2R_Cb23, AT2R or AT1R, were incubated with 0.05 nM ¹²⁵I-[Sarl, Ile8]-Ang II. The ligand competition assay was performed in a total volume of 100 μL assay buffer (50 mM Tris, 5 mM MgCl₂, 1 mM EDTA, 0.1% BSA, pH 7.4), at concentrations ranging from 1 μM to 10 μM. For each experiment, each ligand concentration was tested in duplicate. Non-specific binding (NSB) was determined by the inclusion of 10 μM unlabeled [Sarl]-Ang II (Sigma Aldrich). The reaction was initiated by the addition of radioligand, after which the plates were incubated at 25° C. for one hour. The reaction was terminated by rapid filtration using a vacuum harvester, applying six washes with 100 μL of ice-cold wash buffer (50 mM Tris.HCl, pH 7.4). The filter plates GF/C (Perkin Elmer) were pre-soaked in 0.5% PEI. The residual amount of radioactivity was determined via liquid scintillation counting. IC50 values, representing the concentration at which each ligand displaced 50% of ¹²⁵I-[Sarl, Ile8]-Ang II, were calculated using GraphPad Prism 7.02 by applying a Non-linear regression equation (variable slope, four parameters) on the data. The obtained values serve as affinity estimates for each ligand and their ability to compete against the radioligand for binding with AT2R or AT1R, respectively.

The results are shown in Table 3 (compounds numbered A-01 to A-249) below.

Table 4 shows binding data of compounds of the invention on AT1R, which together with the AT2R binding data presented herein demonstrates their selectivity towards AT2R

TABLE 3
In vitro hAT2R Cb23 binding affinity of
the compounds of the invention, IC50 (M)

| Compound | Binding affinity on |
|---|---|
| number | hAT2R_Cb23 IC50 (M) |
| A-01 | 3.36E−05 |
| A-02 | 6.21E−06 |
| A-03 | 1.59E−05 |
| A-04 | 2.28E−05 |
| A-05 | 3.41E−05 |
| A-06 | 1.65E−05 |
| A-07 | 2.88E−05 |
| A-08 | 6.82E−06 |
| A-09 | 2.18E−06 |
| A-10 | 3.36E−06 |
| A-11 | 9.59E−07 |
| A-12 | 1.27E−06 |
| A-13 | 1.36E−05 |
| A-14 | 4.82E−06 |
| A-15 | 4.29E−05 |
| A-16 | 2.17E−05 |
| A-17 | 1.43E−05 |
| A-18 | 3.85E−06 |
| A-19 | 1.17E−06 |
| A-20 | 5.10E−07 |
| A-21 | 4.88E−07 |
| A-22 | 1.97E−05 |
| A-23 | 2.37E−06 |
| A-24 | 7.14E−06 |
| A-25 | 8.31E−06 |
| A-26 | 1.11E−04 |
| A-27 | 3.59E−05 |
| A-28 | 3.56E−06 |
| A-29 | 4.24E−05 |
| A-30 | 1.14E−04 |
| A-31 | 2.07E−06 |
| A-32 | 1.47E−05 |
| A-33 | 3.07E−06 |
| A-34 | 4.84E−07 |
| A-35 | 6.02E−07 |
| A-36 | 1.42E−06 |
| A-37 | 6.58E−06 |
| A-38 | 2.40E−06 |
| A-39 | 2.81E−06 |
| A-40 | 5.87E−06 |
| A-41 | 2.54E−06 |
| A-42 | 1.77E−05 |
| A-43 | 4.38E−06 |
| A-44 | 7.18E−06 |
| A-45 | 2.54E−06 |
| A-46 | 1.79E−06 |
| A-47 | 1.07E−06 |
| A-48 | 6.28E−07 |
| A-49 | 2.34E−06 |
| A-50 | 1.05E−05 |
| A-51 | 2.11E−06 |
| A-52 | 4.42E−06 |
| A-53 | 7.60E−06 |
| A-54 | 2.07E−06 |
| A-55 | 2.27E−06 |
| A-56 | 6.17E−05 |
| A-57 | 1.90E−06 |
| A-58 | 1.72E−05 |
| A-59 | 4.75E−06 |
| A-60 | 3.46E−06 |
| A-61 | 1.47E−05 |
| A-62 | 1.68E−06 |
| A-63 | 2.16E−05 |
| A-64 | 1.56E−06 |
| A-65 | 1.85E−06 |
| A-66 | 1.18E−05 |
| A-67 | 7.11E−06 |
| A-68 | 8.50E−06 |
| A-69 | 1.06E−05 |
| A-70 | 1.80E−06 |
| A-71 | 1.85E−05 |
| A-72 | 8.39E−06 |
| A-73 | 4.30E−06 |
| A-74 | 1.17E−05 |
| A-75 | 5.92E−06 |
| A-76 | 5.57E−06 |
| A-77 | 2.91E−06 |
| A-78 | 1.50E−06 |
| A-79 | 4.72E−06 |
| A-80 | 2.22E−07 |
| A-81 | 1.84E−06 |
| A-82 | 3.20E−06 |
| A-83 | 1.78E−06 |
| A-84 | 2.37E−06 |
| A-85 | 2.03E−06 |
| A-86 | 2.49E−05 |
| A-87 | 3.81E−06 |
| A-88 | 1.55E−05 |
| A-89 | 9.78E−06 |
| A-90 | 2.03E−06 |
| A-91 | 2.37E−05 |
| A-92 | 1.56E−05 |
| A-93 | 6.98E−06 |
| A-94 | 4.77E−06 |
| A-95 | 4.33E−05 |
| A-96 | 1.32E−05 |
| A-97 | 7.57E−07 |
| A-98 | 5.69E−06 |
| A-99 | 3.39E−05 |
| A-100 | 1.65E−05 |
| A-101 | 4.48E−06 |
| A-102 | 7.07E−06 |
| A-103 | 1.22E−05 |
| A-104 | 6.00E−06 |
| A-105 | 2.00E−06 |
| A-106 | 3.55E−08 |
| A-107 | 5.49E−05 |
| A-108 | 4.17E−05 |
| A-109 | 4.04E−05 |
| A-110 | 1.78E−05 |
| A-111 | 5.43E−06 |
| A-112 | 6.97E−07 |
| A-113 | 7.07E−06 |
| A-114 | 1.87E−05 |
| A-115 | 1.98E−06 |
| A-116 | 8.98E−06 |
| A-117 | 5.26E−05 |
| A-118 | 1.00E−06 |
| A-119 | 9.42E−06 |
| A-120 | 4.59E−07 |
| A-121 | 2.78E−06 |
| A-122 | 2.93E−07 |
| A-123 | 4.88E−06 |
| A-124 | 1.81E−05 |
| A-125 | 3.89E−07 |
| A-126 | 2.26E−05 |
| A-127 | 7.44E−08 |
| A-128 | 2.19E−06 |
| A-129 | 1.58E−07 |
| A-130 | 2.88E−05 |
| A-131 | 1.33E−05 |
| A-132 | 3.55E−07 |
| A-133 | 1.19E−06 |
| A-134 | 9.66E−06 |
| A-135 | 2.20E−08 |
| A-136 | 1.21E−08 |
| A-137 | 1.60E−08 |
| A-138 | 4.71E−06 |
| A-139 | 4.96E−06 |
| A-140 | 7.36E−07 |
| A-141 | 2.27E−05 |
| A-142 | 9.11E−06 |
| A-143 | 5.46E−06 |
| A-144 | 1.48E−05 |
| A-145 | 8.22E−06 |
| A-146 | 9.74E−06 |
| A-147 | 1.47E−06 |
| A-148 | 6.24E−06 |
| A-149 | 8.40E−06 |
| A-150 | 3.38E−05 |
| A-151 | 6.79E−06 |
| A-152 | 2.87E−06 |
| A-153 | 8.42E−06 |
| A-154 | 1.60E−07 |
| A-155 | 7.28E−05 |
| A-156 | 1.16E−05 |
| A-157 | 3.44E−05 |
| A-158 | 4.40E−05 |
| A-159 | 3.08E−06 |
| A-160 | 4.94E−06 |
| A-161 | 1.46E−06 |
| A-162 | 2.07E−05 |
| A-163 | 3.32E−06 |
| A-164 | 1.30E−06 |
| A-165 | 6.81E−07 |
| A-166 | 2.21E−05 |
| A-167 | 3.26E−08 |
| A-168 | 1.77E−08 |
| A-169 | 2.53E−08 |
| A-170 | 1.21E−05 |
| A-171 | 1.58E−08 |
| A-172 | 3.52E−06 |
| A-173 | 1.60E−08 |
| A-174 | 2.35E−09 |
| A-175 | 3.22E−07 |
| A-176 | 2.87E−08 |
| A-177 | 5.85E−07 |
| A-178 | 3.35E−08 |
| A-179 | 9.53E−07 |
| A-180 | 3.95E−09 |
| A-181 | 1.48E−08 |
| A-182 | 1.86E−08 |
| A-183 | 8.33E−09 |
| A-184 | 8.93E−10 |
| A-185 | 6.96E−08 |
| A-186 | 1.83E−08 |
| A-187 | 7.26E−09 |
| A-188 | 1.39E−08 |
| A-189 | 2.65E−06 |
| A-190 | 1.35E−06 |
| A-191 | 5.31E−08 |
| A-192 | 5.19E−07 |
| A-193 | 3.89E−07 |
| A-194 | 7.37E−07 |
| A-195 | 5.37E−07 |
| A-196 | 6.31E−07 |
| A-197 | 2.03E−07 |
| A-198 | 1.82E−06 |
| A-199 | 1.76E−07 |
| A-200 | 1.06E−06 |
| A-201 | 6.50E−07 |
| A-202 | 8.24E−07 |
| A-203 | 1.55E−07 |
| A-204 | 2.12E−07 |
| A-205 | 3.79E−07 |
| A-206 | 1.08E−06 |
| A-207 | 1.21E−06 |
| A-208 | 1.92E−06 |
| A-209 | 3.68E−06 |
| A-210 | 2.01E−06 |
| A-211 | 1.29E−06 |
| A-212 | 4.34E−07 |
| A-213 | 3.89E−07 |
| A-214 | 3.86E−08 |
| A-215 | 3.00E−07 |
| A-216 | 2.47E−07 |
| A-217 | 2.79E−09 |
| A-218 | 1.22E−07 |
| A-219 | 6.81E−07 |
| A-220 | 8.07E−08 |
| A-221 | 2.74E−06 |
| A-222 | 9.47E−09 |
| A-223 | 6.00E−09 |
| A-224 | 9.22E−08 |
| A-225 | 5.41E−08 |
| A-226 | 3.72E−08 |
| A-227 | 8.75E−09 |
| A-228 | 1.49E−08 |
| A-229 | 7.98E−09 |
| A-230 | 1.23E−06 |
| A-231 | 3.68E−07 |
| A-232 | 2.67E−06 |
| A-233 | 1.31E−06 |
| A-234 | 2.02E−06 |
| A-235 | 6.89E−06 |
| A-236 | 1.01E−06 |
| A-237 | 4.42E−07 |
| A-238 | 1.88E−05 |
| A-239 | 2.57E−06 |
| A-240 | 3.62E−05 |
| A-241 | 4.25E−05 |
| A-242 | 2.77E−05 |
| A-243 | 1.59E−07 |
| A-244 | 1.04E−07 |
| A-245 | 9.97E−06 |
| A-246 | 1.02E−07 |
| A-247 | 3.53E−05 |
| A-248 | 4.80E−08 |
| A-249 | 7.90E−05 |

TABLE 4
In vitro hAT1R WT binding affinity of the
compounds of the invention, IC50 (M)

| | % Displaced | % Displaced | Binding affinity |
|---|---|---|---|
| Compound | radioligand | radioligand | On hAT1R IC50 |
| number | at 1 μM | at 10 μM | (M) |
| A-02 | * | * | |
| A-03 | * | * | |
| A-05 | * | * | 4.99E−05 |
| A-06 | * | ** | 2.54E−05 |
| A-08 | * | * | 9.21E−05 |
| A-09 | * | * | 3.65E−05 |
| A-10 | * | * | 7.00E−05 |
| A-11 | * | * | 3.77E−05 |
| A-12 | * | * | |
| A-13 | * | * | |
| A-14 | * | * | |
| A-17 | * | * | |
| A-18 | * | * | |
| A-19 | * | * | 4.74E−05 |
| A-20 | * | * | 4.97E−05 |
| A-21 | * | * | 4.17E−05 |
| A-22 | * | * | |
| A-23 | * | * | |
| A-24 | * | * | |
| A-25 | * | * | |
| A-28 | * | * | |
| A-31 | * | * | |
| A-32 | * | * | |
| A-33 | * | * | |
| A-34 | * | * | |
| A-35 | * | * | 3.14E−05 |
| A-36 | * | * | |
| A-37 | * | * | |
| A-38 | * | ** | 1.36E−05 |
| A-39 | * | * | |
| A-40 | * | * | 3.66E−05 |
| A-41 | * | ** | 1.65E−05 |
| A-42 | * | ** | 2.63E−05 |
| A-43 | * | ** | |
| A-44 | * | * | 3.49E−05 |
| A-45 | * | * | 3.00E−05 |
| A-46 | * | * | |
| A-47 | * | * | |
| A-48 | * | ** | 2.78E−05 |
| A-49 | * | ** | 2.03E−05 |
| A-50 | * | */** | 2.88E−05 |
| A-51 | * | * | 5.50E−05 |
| A-52 | * | * | |
| A-53 | * | * | 4.56E−05 |
| A-54 | * | * | 3.10E−05 |
| A-55 | * | * | 3.97E−05 |
| A-57 | * | * | 3.45E−05 |
| A-59 | * | * | |
| A-60 | * | * | |
| A-62 | * | * | |
| A-64 | * | * | 3.97E−05 |
| A-65 | * | * | 6.08E−05 |
| A-67 | * | * | |
| A-68 | * | * | |
| A-69 | * | * | |
| A-70 | * | * | 6.12E−05 |
| A-72 | * | * | |
| A-73 | * | * | |
| A-75 | * | * | |
| A-76 | * | * | |
| A-77 | * | ** | 1.35E−05 |
| A-78 | * | ** | 1.22E−05 |
| A-79 | * | * | |
| A-80 | * | * | |
| A-81 | * | * | |
| A-82 | * | * | |
| A-83 | * | * | |
| A-84 | * | * | |
| A-85 | * | * | 4.50E−05 |
| A-86 | * | * | |
| A-87 | * | * | |
| A-88 | * | * | |
| A-89 | * | * | |
| A-90 | * | * | |
| A-93 | * | * | |
| A-94 | * | ** | 1.42E−05 |
| A-97 | * | * | |
| A-98 | * | * | |
| A-101 | * | * | |
| A-102 | * | * | |
| A-104 | * | * | |
| A-105 | * | * | |
| A-106 | * | * | |
| A-111 | * | * | |
| A-112 | * | * | |
| A-113 | * | * | |
| A-115 | * | * | |
| A-116 | * | ** | 1.81E−05 |
| A-118 | * | * | 4.15E−05 |
| A-119 | * | * | |
| A-120 | * | * | |
| A-121 | * | ** | 1.17E−05 |
| A-122 | * | * | |
| A-123 | * | * | |
| A-125 | * | * | |
| A-127 | * | * | |
| A-128 | ** | *** | 1.82E−05 |
| A-129 | * | * | |
| A-132 | * | * | |
| A-133 | * | * | |
| A-134 | * | * | |
| A-135 | * | * | |
| A-136 | * | * | |
| A-137 | * | * | |
| A-138 | * | * | |
| A-139 | * | * | |
| A-140 | * | ** | 2.47E−05 |
| A-142 | * | ** | |
| A-143 | * | * | |
| A-145 | * | * | |
| A-146 | * | * | |
| A-147 | * | * | |
| A-148 | * | * | |
| A-149 | * | * | |
| A-151 | * | * | |
| A-152 | * | * | |
| A-153 | * | * | |
| A-154 | * | * | |
| A-159 | * | * | |
| A-160 | * | * | |
| A-161 | * | * | |
| A-163 | * | * | |
| A-164 | * | * | |
| A-165 | * | * | |
| A-167 | * | * | |
| A-168 | * | * | |
| A-169 | * | * | |
| A-170 | * | * | |
| A-171 | * | * | |
| A-172 | * | * | |
| A-173 | * | * | |
| A-174 | * | * | |
| A-175 | * | * | |
| A-176 | * | * | |
| A-177 | * | * | |
| A-178 | * | * | |
| A-179 | * | * | |
| A-180 | * | * | |
| A-181 | * | * | |
| A-182 | * | * | |
| A-183 | * | * | |
| A-184 | * | * | 3.36E−05 |
| A-185 | * | * | |
| A-186 | * | * | |
| A-187 | * | * | |
| A-188 | * | * | |
| A-189 | * | * | |
| A-190 | * | * | |
| A-191 | * | * | |
| A-192 | * | * | |
| A-193 | * | * | |
| A-194 | * | * | |
| A-195 | * | * | |
| A-196 | * | * | |
| A-197 | * | * | |
| A-198 | * | * | |
| A-199 | * | * | |
| A-200 | * | * | |
| A-201 | * | * | |
| A-202 | * | * | |
| A-203 | * | * | |
| A-204 | * | * | |
| A-205 | * | * | |
| A-206 | * | * | |
| A-207 | * | * | |
| A-208 | * | * | |
| A-209 | ** | *** | 6.74E−06 |
| A-210 | * | * | |
| A-211 | * | * | |
| A-212 | * | * | |
| A-213 | * | * | |
| A-214 | * | * | |
| A-215 | * | * | |
| A-216 | * | * | |
| A-217 | * | ** | 1.75E−05 |
| A-218 | * | * | |
| A-219 | * | * | 5.52E−05 |
| A-220 | * | * | |
| A-221 | * | * | |
| A-222 | * | * | |
| A-223 | * | * | |
| A-224 | * | * | |
| A-225 | * | * | |
| A-226 | * | * | |
| A-227 | * | * | |
| A-228 | * | * | |
| A-229 | * | * | |
| A-230 | * | * | |
| A-231 | * | ** | 1.89E−05 |
| A-232 | * | * | |
| A-233 | * | * | |
| A-234 | * | * | |
| A-235 | * | * | |
| A-236 | * | * | |
| A-239 | * | * | |
| A-243 | * | *** | 1.15E−05 |
| A-244 | * | *** | 6.62E−06 |
| A-246 | * | ** | 2.67E−05 |
| A-248 | * | * | 4.01E−05 |

Semi-quantitative score % Displaced radioligand 125I-[Sar1, Ile8]-Ang II
* <30%
** 30%-50%
*** >50%

### Example 3: In Vivo Data (CCI Model of Neuropathic Pain)

The compounds of the invention and a reference compound (EMA-200) were tested in a rat model of neuropathic pain (Bennett and Xie (1988) rat chronic constriction injury (CCI) model of mononeuropathic pain: A peripheral mononeuropathy in rat that produces disorders of pain sensation like those seen in man. Bennett G J, Xie Y K. Pain. 1988:87-107).

The results are shown/summarized in FIGS. 1 and 2.

### Example 4: Selectivity Profiling (Broad Panel)

The purpose of this assay is to determine the activity and selectivity of a compound of the invention on a selected range of human GPCRs, ion channels, kinases, transporters, etc. which may result in undesirable side-effects when inhibited.

Binding affinity or activity towards these targets is determined in radiometric, enzyme and cell-based assays at Eurofins Cerep SA (Le Bois L'Eveque, BP 30001, F-86600 Celle-Levescault). To determine its EC50, IC50 or PIN (percentage inhibition) a compound is tested in a single concentration of 10 μM (highest concentration). The results for the tested compounds of the invention (data not shown) showed that the tested compounds have high selectivity towards the target of interest, i.e. AT2R and have minimum to no activity on the receptors tested in the selectivity assay.

In addition, a representative compound of the invention was tested for activity on the following targets: at Eurofins Cerep SA, Eurofins DisoverX and Charles River Laboratories Cleveland, Inc.: agonist and antagonist activity, respectively, against AT1R in calcium flux ((EC50>100 micromolar and IC50>100 micromolar, respectively); CGRP (no significant antagonist effect—data not shown); TRPV1 (no significant antagonist effect—data not shown); TPRA1 (no agonist or antagonist effect beyond threshold levels—data not shown) and NK3 (no significant stimulating or inhibitory effect—data not shown).

### Example 5: ADME Assays

### Kinetic Solubility

Starting from a 20 mM stock solution of test compound in 100% DMSO, dilutions were prepared to a theoretical concentration of 400 μM in duplicates in phosphate-buffered saline pH 7.4 (PBS) (138 mM NaCl, 2.7 mM KCl, 10 mM K-phosphate) and 100 mM citrate buffer pH 3.0 with 2% final DMSO.

The experimental compound dilutions in PBS and citrate buffer were further allowed to equilibrate at 25° C. on a thermostatic orbital shaker for two hours and then centrifuged at 14000 rpm, 5 min and filtered through HTS filter plates using a vacuum manifold. The filtrates of test compounds were diluted 2-fold with acetonitrile with 2% DMSO before measuring. In parallel, compound dilutions in 50% acetonitrile/buffer were prepared to the theoretical concentrations of 0 μM (blank), 100 μM, 200 μM and 400 μM with 2% final DMSO to generate calibration curves.

Ondansetron (Sigma Aldrich USA, cat #03639) was used as a reference to control proper assay performance. 200 μl of each sample were transferred to a 96-well plate and solubility was measured in a 200-550 nm range (in steps of 5 nm). The measurements were performed using a Spectra Max Plus reader in UV-Vis mode. Acquisition and analysis of the data were performed using SoftMax Pro v.5.4 (Molecular Devices) and Excel 2010 data analysis software. Proper absorbance wavelengths for calculations were selected for each compound manually based on absorbance maximums (absolute absorbance unit values for the minimum and maximum concentration points within 0-3 OD range). Each of the final datasets was additionally visually evaluated by the operator and goodness of fit (R2) is calculated for each calibration curve.

The results are shown in Table 5.

TABLE 5
Kinetic solubility of illustrative compounds of the invention

| Compound | PBS solubility, | Citrate buffer |
|---|---|---|
| number | pH 7.4, μM | solubility, pH 3.0, μM |
| A-09 | 318 | ≥400 |
| A-10 | ≥400 | ≥400 |
| A-12 | 54 | ≥400 |
| A-20 | ≥400 | ≥400 |
| A-24 | ≥400 | ≥400 |
| A-31 | ≥400 | 40 |
| A-32 | 394 | 399 |
| A-51 | ≥400 | 4 |
| A-90 | ≥400 | ≥400 |
| A-173 | 376 | 356 |
| A-174 | 385 | 356 |
| A-178 | ≥400 | ≥400 |
| A-181 | ≥400 | 388 |
| A-183 | 383 | 377 |

### Thermodynamic Solubility

### Thermodynamic Solubility Protocol 1

In a 8 mL glass vial, 1-2 mg of dry matter of the test compound were mixed with the suitable buffers (phosphate-buffered saline pH 7.4 (138 mM NaCl, 2.7 mM KCl, 10 mM K-phosphate) or 100 mM citrate buffer pH 3.0) to reach a theoretical concentration of 4 mM. The solutions were allowed to further equilibrate at 25° C. on a thermostatic shaker. After 4 and 24 hours of shaking, respectively, the incubation mixtures were filtered through HTS filter plates using a vacuum manifold. The filtrates of test compounds were diluted 2-fold with acetonitrile with 4% DMSO before measuring. In parallel, using a 20 mM DMSO stock solution, compound dilutions in 50% acetonitrile/buffer were prepared to the theoretical concentrations of 0 μM (blank), 10 μM, 25 μM, 50 μM, 100 μM and 200 μM with 2% final DMSO to generate calibration curves.

Ondansetron (Sigma Aldrich USA, cat #03639) was used as a reference to control proper assay performance. 200 μl of each sample were transferred to a 96-well plate and solubility was measured in a 200-550 nm range (in steps of 5 nm). The measurements were performed using a Spectra Max Plus reader in UV-Vis mode. Acquisition and analysis of the data were performed using SoftMax Pro v.5.4 (Molecular Devices) and Excel 2010 data analysis software. Proper absorbance wavelengths for calculations were selected for each compound manually based on absorbance maximums (absolute absorbance unit values for the minimum and maximum concentration points within 0-3 OD range). Each of the final datasets was additionally visually evaluated by the operator and goodness of fit (R₂) is calculated for each calibration curve.

### Thermodynamic Solubility Protocol 2

In a 8 mL glass vial, 1-2 mg of dry matter of the test compound were mixed with the suitable buffers (Fed State Simulated Intestine Fluid (FeSSIF) or Fasted State Simulated Intestine Fluid (FaSSIF)) to reach a theoretical concentration of 4 mM. The solutions were allowed to further equilibrate at 25° C. on a thermostatic shaker. After 4 and 24 hours of shaking, respectively, the incubation mixtures were filtered through HTS filter plates using a vacuum manifold. The filtrates of test compounds were diluted 500-fold with acetonitrile/buffer mixtures with 2% DMSO before measuring. In parallel, using a 20 mM DMSO stock solution, compound dilutions in 50% acetonitrile/buffer (v/v) were prepared to the theoretical concentrations of 0 μM (blank), 10 μM, 25 μM, 50 μM, 100 μM and 200 μM with 2% final DMSO to generate calibration curves. Calibration standards were diluted 100-fold with 50% acetonitrile/water (v/v) mixes before LC-MS/MS measurement (systems API4000 QTRAP).

The results are shown in Table 6.

TABLE 6
Thermodynamic solubility of illustrative
compounds of the invention

| | | | | TSOL | TSOL |
|---|---|---|---|---|---|
| | | TSOL | TSOL | FaSSIF | FeSSIF |
| Compound | Time | pH 7.4 | pH 3.0 | pH 6.5 | pH 5.0 |
| number | point | (μM) | (μM) | (μM) | (μM) |
| A-174 | 4 h | >400 | ≥400 | >400 | >400 |
| | 24 h | >400 | ≥400 | >400 | >400 |

### Plasma Protein Binding PPB (Equilibrium Dialysis)

### PPB—Protocol 1

The assay was performed in a multiple-use 96-well dialysis unit (HTD96b dialyzer). Each individual well unit consisted of 2 chambers separated by a vertically aligned dialysis membrane of predetermined pore size (MWCO 12-14 kDa). 120 μl of non-diluted freshly thawed mouse, rat or human plasma spiked with the compound (1 μM, final DMSO concentration 1%) was added to one chamber and the same volume of PBS buffer pH 7.4 to the other chamber. HTD96b dialyzer was covered with adhesive sealing film and incubated at 37° C., shaking at 100 rpm for 5 hours.

For samples preparation, an aliquot of the content of each chamber had been mixed with the same volume of the blank opposite matrix. In order to define non-specific loss of the compound during this assay, a standard solution was created by mixing an aliquot of spiked plasma with blank buffer without dialysis. Samples were diluted 10-fold with 100% acetonitrile with subsequent plasma proteins sedimentation by centrifuging at 6000 rpm for 5 minutes. Supernatants were analyzed using HPLC system coupled with tandem mass spectrometer (API3000 PE Sciex). Acquisition and analysis of the data were performed using Analyst 1.5.2 software (PE Sciex). The percentage of plasma protein bound compound and recovery were calculated using following equations:

$$protein binding = ( 1 - \frac{peak area in buffer}{peak area in plasma} ) * 100 \%$$

$$recovery = ( \frac{peak area in buffer + peak area in plasma}{peak area in standard solution} ) * 100 \%$$

### PPB—Protocol 2

Prior to the start of the experiment, dialysis membranes (membrane strips, MW cut-off 12-14 kDa, HTDialysis, Cat.NoJl 101) are soaked in deionized water for 60 min, transferred and left overnight in 20% EtOH. The day of experiment, a 10 mM stock solution of the compound in DMSO is diluted with a factor 10 in DMSO. This solution is further diluted in freshly thawed human, rat, mouse or dog plasma (BioIVT) with a final concentration of 5 μM and final DMSO concentration of 0.5%.

From this solution, an aliquot of 50 μL was taken and matrix matched with an equivalent volume of PBS for the recovery plate, after which 6 volumes of STOP solution were added to the recovery plate. For these recovery plates, no incubation is done.

Equilibrium Dialysis Device (96-well, model HTD96b, HTDialysis, Cat. No. #1006) is assembled according to manufacturer's instructions. Immediately after assembly, a volume of 100 μL of plasma (spiked with compound) is placed on one side of the well and another 100 μL of blank PBS buffer are added to the other side, respectively. Each compound is tested in duplicate. Acebutolol and Nicardipine are used as low and very high binding controls, except for the mouse, Caffeine is used as low binder instead Acebutolol. If the PPB values for these controls are not in the range determined by the historical data, the assay is not validated.

The plate is incubated for 4 h at 37° C. while shaking at 230 rpm. Thereafter, an aliquot of 50 μL is taken from each side of the well and matrix matched (mix of equal volumes of spiked plasma with blank PBS buffer and samples from buffer compartment with blank plasma). Matrix matched samples are further mixed with 3 volumes of STOP solution (acetonitrile/MeOH 2/1 with diclofenac as internal standard). After brief mixing and centrifugation (at 2400 rpm for 15 min, at +4° C.), the supernatant is filtered and transferred into new 96-well plates for analysis on LC-MS/MS (systems API4000).

The percentage of plasma protein bound compound and recovery were calculated using following equations:

$$plasma protein binding = ( \frac{Cplasma - Cbuffer}{Cplasma} ) * 100 \%$$

with:
Cₚₗₐₛₘₐ=peak area of the compound in the plasma/peak area of the IS in the plasma
C_{buffer}=peak area of the compound in the buffer/peak area of the IS in the buffer
‘Concentration’ is the ratio between compound and IS peak areas

The recovery is a control, which allows to be sure that the compound has not a non-specific binding to the plates or is not stable in the plasma in these conditions:

$$\% recovery = \frac{( PBS + Plasma ) * 100}{Recov}$$

with:
PBS=(ratio of the peak area of the compound/peak area of the IS) in the PBS compartment after 4 h
Plasma=(ratio of the peak area of the compound/peak area of IS) in the plasma compartment after 4 h
Recov=Recovery=ratio of the peak are of the compound I the well recovery/peak area of the IS in the well recovery at TO

The solubility of the compound in the final test concentration in PBS is checked by microscope to indicate whether precipitation is observed or not. If a precipitate is observed, no data of PPB is generated.

The results are shown in Table 7.

TABLE 7
PPB of illustrative compounds of the invention

| | PPB | Recovery | PPB | Recovery | PPB | Recovery | PPB | Recovery |
|---|---|---|---|---|---|---|---|---|
| Compound | (%), | (%), | (%), | (%), | (%), | (%), | (%), | (%), |
| number | mouse | mouse | rat | rat | dog | dog | human | human |
| A-31 | >95 | 88 | >90 | 84 | | | >92 | 82 |
| A-90 | 78 | 97 | 77 | 105 | | | 95 | 83 |
| A-173 | 88 | 92 | 81 | 100 | | | 95 | 94 |
| A-174 | 97.3 | 95 | 98.3 | 90 | 93.8 | 97 | 97.4 | 99 |
| A-178 | 90 | 93 | 88 | 96 | | | 96 | 99 |
| A-181 | 93 | 96 | 88 | 96 | | | 97 | 95 |
| A-183 | 87 | 90 | 77 | 92 | | | 94 | 96 |
| A-217 | | | 99.6 | 98 | 95.8 | 96 | 99.6 | 100 |

### Liver Microsomal Stability

### LMS—Protocol 1

Mouse hepatic microsomes were isolated from pooled (50), perfused livers of male Balb/c mice. Rat hepatic microsomes were isolated from pooled (15), perfused livers of male Wistar rats. Isolation was performed according to the standard protocol (Hill, J. R. in Current Protocols in Pharmacology 7.8.1-7.8.11, Wiley Interscience, 2003). The batches of microsomes were tested for quality control using Imipramine, Propranolol and Verapamil as reference compounds.

Microsomal incubations were carried out in 96-well plates in 5 aliquots of 40 μL each (one for each time point). Liver microsomal incubation medium contained PBS (100 mM, pH 7.4), MgCl₂ (3.3 mM), NADPH (3 mM), glucose-6-phosphate (5.3 mM), glucose-6-phosphate dehydrogenase (0.67 units/ml) with 0.42 mg of liver microsomal protein per ml. Control incubations were performed replacing the NADPH-cofactor system with PBS. Test compound (2 μM, final solvent concentration 1.6%) was incubated with microsomes at 37° C., shaking at 100 rpm. Incubations were performed in duplicates. Five time points over 40 minutes have been analyzed. The reactions were stopped by adding 12 volumes of 90% acetonitrile-water to incubation aliquots, followed by protein sedimentation by centrifuging at 5500 rpm for 3 minutes. Incubations were performed in duplicates. Supernatants were analyzed using the HPLC system coupled with tandem mass spectrometer (API3000 PE Sciex). Acquisition and analysis of the data were performed using Analyst 1.5.2 software (PE Sciex). The elimination constant (kel), half-life (t½) and intrinsic clearance (Clint) were determined in plot of ln(AUC) versus time, using linear regression analysis:

$$\begin{matrix}{k}_{el} = - slope & {t}_{1 / 2} = \frac{0.693}{k} & {CL}_{int} = \frac{0.693}{{t}_{1 / 2}}\end{matrix} \times \frac{μ L incubation}{mg microsomes}$$

### LMS—Protocol 2

Mouse hepatic microsomes were isolated from pooled, perfused livers of male CD1 mice. Rat hepatic microsomes were isolated from pooled, perfused livers of male Sprague Dawley rats. Dog hepatic microsomes were isolated from pooled, perfused livers of male Beagle dogs. The batches of microsomes were tested for quality control using Testosterone, Propranolol and Caffeine as reference compounds.

A 10 mM stock solution of compound in DMSO is diluted three-fold in DMSO. This pre-diluted compound solution is then further diluted to 2 μM in a 50 mM phosphate buffer (PBS, pH 7.4) and pre-warmed at 37° C. This compound dilution is mixed F₂ with microsomal/cofactor mix at 37° C. under shaking at 300 rpm. Final reaction conditions are: 100 μL incubation volume, 1 μM of test compound (in duplicate), <0.1% DMSO, 0.5 mg/mL microsomes, 0.6 U/mL Glucose-6-phosphate-dehydrogenase (G6PDH, Roche, 10127671001), 3.3 mM MgCl₂ (Sigma, M2670), 3.3 mM glucose-6-phosphate (Sigma, G-7879) and 1.3 mM NADP+ (Sigma, N-0505).

After 60 min of incubation at 300 rpm and 37° C., the reaction was stopped with 3 volumes of STOP solution (acetonitrile/MeOH 2/1 with diclofenac as internal standard). For the time point zero, 3 volumes of STOP solution were added to the compound dilution before the microsome mix was added. Six time points over 60 minutes have been analyzed. The samples of all time points were centrifuged, filtered and the supernatant analyzed by LCMS/MS (systems API4000).

The instrument responses (peak areas/IS peak area) were referenced to the zero time-point samples (considered as 100%) in order to determine the percentage of compound remaining.

Testosterone (1 μM) and Propranolol and Caffeine (1 μM) were used as reference compounds, being unstable and stable compounds respectively. If the microsomal stability values for these controls are not in the range determined by the historical data, the assay is not validated. The solubility of the compound in the final test concentration in 50 mM buffer pH 7.4 is checked by microscope to indicate whether precipitation is observed or not. If a precipitate is observed, no data of microsomal stability is generated.

The results are shown in Table 8.

TABLE 8
Liver microsomal stability of illustrative
compounds of the invention

| Compound | Mouse Clint | Rat Clint | Dog Clint | Human Clint |
|---|---|---|---|---|
| number | (μL/min/mg) | (μL/min/mg) | (μL/min/mg) | (μL/min/mg) |
| A-09 | 33 | 33 | | 37 |
| A-10 | 69 | 28 | | 99 |
| A-12 | 157 | 23 | | 259 |
| A-20 | 20 | 28 | | 24 |
| A-24 | 9 | 7 | | 8 |
| A-31 | 16 | 5 | | 8 |
| A-32 | 6 | 13 | | 10 |
| A-48 | 14 | 13 | | 32 |
| A-51 | 4 | 1 | | 12 |
| A-82 | 7 | 4 | | 15 |
| A-83 | 11 | 3 | | 6 |
| A-84 | 17 | 7 | | 40 |
| A-88 | 7 | 7 | | 6 |
| A-90 | 10 | 4 | | 7 |
| A-173 | 5 | 12 | | 4 |
| A-174 | 15.3 | <17.3 | <11.7 | <6.6 |
| A-178 | 15 | 5 | | 20 |
| A-181 | 6 | 5 | | 12 |
| A-183 | 12 | 7 | | 7 |
| A-184 | | 19 | | 18 |
| A-217 | | <17.3 | <11.5 | <6.6 |
| A-223 | | 24.1 | | 18.2 |
| A-227 | | 22.3 | <11.7 | 16.5 |
| A-228 | | 28.3 | <11.7 | 97.6 |
| A-229 | | 24.8 | <11.7 | 21.6 |

### Metabolic Stability in Hepatocytes

### LHS—Protocol 1

Primary mouse hepatocytes were isolated from 12-week old male balb/c mice (n=3) by two-step collagenase liver perfusion. Tribromoethanol was used as anesthetic agent at a dose of 250 mg/kg. Mouse liver was first perfused for 3 min with a pre-perfusing solution (HBSS w/o Ca²⁺ and Mg²⁺, 20 mM HEPES pH 7.4, 0.5 mM EDTA), then for 2 min with washing solution (HBSS, 20 mM HEPES pH 7.4), and then for 6 min with perfusing solution (HBSS, 20 mM HEPES pH 7.4, 5 mM CaCl₂, 0.5 mg/ml collagenase Type IV). Flow rate was maintained at 7 mL/min and all solutions were kept at 37° C. After in situ perfusion, the liver was excised, the liver capsule was opened, and cells were suspended in William's Medium E and filtered through a 70 μm membrane. Dead cells were removed by Percoll centrifugation (Percoll density: 1.06 g/mL, 50×g, 10 min, 20° C.) and additional centrifugation in William's Medium E (50×g, 3 min). The cell pellet was resuspended in CryoScarless DMSO free (Cat. No. CPL-A1, BioVerde Inc., Japan) medium (cell density—1×10⁶/mL), frozen at −70° C. and stored in liquid nitrogen.

Primary rat hepatocytes were obtained from male Wistar rats (n=3) by collagenase liver perfusion similar to mouse hepatocytes isolation. Briefly, rat liver was perfused with pre-perfusing solution for 3 min, then for 1 min with washing solution, and then 6 min with the perfusion solution 2 (HBSS, 20 mM HEPES pH 7.4, 5 mM CaCl₂, 0.9 mM MgCl₂ and 0.1 mg/mL collagenase Type II). Flow rate was maintained at 17 mL/min and all solutions were kept at 37° C. All further manipulations were performed as well as for mouse hepatocytes. The cell pellet was resuspended in CryoScarless DMSO free (Cat. No. CPL-A1, BioVerde Inc., Japan) medium (cell density—1×10⁶/mL), frozen at −70° C. and stored in liquid nitrogen.

Prior to use, cells were thawed at 37° C., resuspended in William's Medium E and their viability was determined by Trypan Blue exclusion using a hemocytometer. The batches of hepatocytes were tested for quality control using Imipramine, Propranolol and Testosterone as reference compounds.

10-mM DMSO stock solutions of each drug were diluted to 6 μM (2× concentration) using William's Medium E to create the working samples. Aliquots (50 μL) of the hepatocyte suspension (rat hepatocytes density—0.68×10⁶/mL and mouse hepatocytes density—0.4×10⁶/mL in William's Medium E) were added to each test well of a 96-well plate immediately followed by the addition of 50 μL aliquot of the test compound or control solutions. The samples for each time point (0, 5, 10, 30, 60, and 120 minutes) were prepared in duplicates for all the test and reference compounds. Incubations were done at 37° C., 5% CO₂ and 95% relative humidity in CO₂ incubator. At appropriate time-points, 40 μL aliquots were removed from the wells and placed in 1.1 mL microtubes containing 200 μL of acetonitrile. The samples were centrifuged at 6000 rpm for 4 min and 100 μL aliquots of the supernatants were transferred into new set of microtubes for LC-MS/MS analysis (API 3000 PE Sciex). Acquisition and analysis of the data were performed using Analyst 1.5.2 software (PE Sciex). Elimination constant (kₑₗ), half-life (t_{1/2}) and intrinsic clearance (Clᵢₙₜ) were determined in plots of ln (percent remaining of parent compound) versus time, using linear regression analysis:

$${k}_{el} = - slope \qquad {t}_{1 / 2} = \frac{ln 2}{- k}$$

$${CL}_{int} = \frac{0.693}{{t}_{\frac{1}{2}}} \times \frac{μ L incubation}{number of cells in cubation ( {10}^{6} )} ( μ L / min / {10}^{6} cells )$$

### LHS—Protocol 2

The aim of this assay is to determine the metabolic stability of the compound in hepatocytes (cryopreserved) of different species (mouse CD1, rat Sprague Dawley, dog Beagle). Low hepatocyte stability may result in the formation of unwanted metabolites, high clearance, and therefore is not desirable. The decrease in parent was assessed by measuring the percentage remaining by LC-MS/MS analysis. The batches of hepatocytes were tested for quality control using Testosterone, Umbelliferone and Caffeine as reference compounds.

A 10 mM stock solution of test compound in DMSO was first diluted in DMSO to 3 mM, and then in modified Krebs-Henseleit buffer (Sigma, K3753) to 5 μM. This compound dilution was added to a suspension of pooled cryopreserved hepatocytes (BioIVT) at 37° C. under gentle shaking. Final reaction conditions were: 1 μM of test compound, <0.1% DMSO, 0.5 million viable hepatocytes/mL, and 75 μL incubation volume.

After 0, 10, 20, 45, 90, 120 and 180 min of incubation, the reaction was terminated with 3 volumes of MeCN:MeOH (2:1) containing diclofenac as analytical internal standard. Samples were mixed, centrifuged and the supernatant analyzed by LC-MS/MS (systems API4000). The instrument responses (ratios of test compound and internal standard peak areas) were referenced to the zero time point samples (considered as 100%) in order to determine the percentage of compound remaining. Plots of percentage compound remaining were used to determine the intrinsic clearance in the hepatocyte incubations.

Testosterone (1 μM) and Umbelliferone (1 μM) and Caffeine (1 μM) were used as reference compounds, being unstable and stable compounds respectively. If the hepatocyte stability values for these controls are not in the range determined by the historical data, the assay is not validated.

The results are shown in Table 9.

TABLE 9
Liver hepatocyte stability of illustrative
compounds of the invention

| | Mouse Clint | Rat Clint | Dog Clint | Human Clint |
|---|---|---|---|---|
| Compound | (μL/min/ | (μL/min/ | (μL/min/ | (μL/min/ |
| number | 10⁶ cells) | 10⁶ cells) | 10⁶ cells) | 10⁶ cells) |
| A-173 | 4 | 2 | | |
| A-174 | <5.4 | 10.5 | <5.1 | <2.9 |
| A-178 | 0.8 | 0.2 | | |
| A-181 | 1 | 1 | | |
| A-183 | 2 | 2.6 | | |
| A-184 | | 2 | | |
| A-217 | | 8.7 | <5.1 | 5.8 |

### CYP Inhibition

The aim of this assay is to determine the inhibitory potential of a test compound. A major concern for drug-drug-interaction is cytochrome P450 inhibition. Reversible CYP inhibition was determined in human liver microsomes using specific probe substrates for human cytochrome P450 isoenzymes CYP1A2, 2C9, 2C19, 2D6 and 3A4.

### CYP Inhibition—Protocol 1

The potential for CYP450 inhibition was assessed by performing in vitro inhibition studies using fluorogenic CYP450 substrates with the corresponding CYP450 enzymes and NADPH regeneration system (Vivid CYP450 Screening Kits) with some minor changes to the manufacturer's protocols. The fluorescent signal produced from reaction is directly proportional to the cytochrome P450 activity. In the cases when tested compounds interfere with the CYP450 enzyme-substrate reaction, the fluorescent signal decreases. In brief, the tested compounds were first dissolved in DMSO at 100× concentration (1 mM), and diluted in buffer to 2.5× concentration (25 μM). Then the 2.5× compound solutions were mixed with the Master Pre-mix consisting of Human CYP450+Oxidoreductase and NADP+Regeneration System (glucose-6-phosphate and glucose-6-phosphate dehydrogenase). After 10 minutes pre-incubation, the enzymatic reaction was initiated by the addition of a mix of NADPH and the appropriate CYP450 substrates. The plate was incubated for the desired reaction time (25 min for CYP1A2, CYP2C₉, CYP2D6, and CYP3A4, 60 min for CYP2C₁₉) after which Stop Reagent was added and fluorescence measured using SpectraMax Paradigm Multi-Mode Microplate Reader. All test points were performed in quadruplicates at concentration 10 μM (1% DMSO). The results are given in Table 12.

TABLE 10
Reference compounds used to assess CYP inhibition

| | | Ref. inhibitor | |
|---|---|---|---|
| CYP | Reference inhibitor | conc. (μM) | % Inhibition |
| 1A2 | α-naphthoflavone | 2 | 101.61 |
| 2C9 | sulfaphenazole | 5 | 97.85 |
| 2C19 | ticlopidine | 25 | 99.06 |
| 2D6 | quinidine | 0.5 | 87.31 |
| 3A4 | ketoconazole | 2 | 99.35 |

### CYP Inhibition—Protocol 2

A 10 mM stock solution of test compound is prepared in methanol. This stock is further serially diluted 1:3 in methanol and then added to mixture containing 50 mM potassium phosphate buffer pH7.4, human liver microsomes (BD Gentest) and probe substrate. After pre-warming 5 min at 37° C., the reaction is started by adding co-factor mix (7.65 mg/mL glucose-6-phosphate, 1.7 mg/mL NADP, 6 U/mL of glucose-6-phosphate dehydrogenase), resulting in a final concentration of test compound at 10 μM (2% MeOH). Table 11 summarizes the assay conditions used.

Final concentrations of co-factor mix components are as follows: 1.56 mg/mL glucose-6-phosphate, 0.34 mg/mL NADP, 1.2 U/mL of glucose-6-phosphate dehydrogenase.

After incubation at 37° C., the reaction (aliquot of 50 μL) is terminated with 150 μL CH₃CN:MeOH (2:1) solution with internal standard (warfarin for 2C₉, diclofenac for all other tested isoforms). Samples are centrifuged and the supernatant fractions analyzed by LC-MS/MS.

The instrument responses (ratio of test compound and internal standard peak areas) are referenced to those for solvent controls (assumed as 100%) in order to determine the percentage reduction in probe metabolism. Percent of control activity vs. concentration plots are generated and fitted using GraphPad Prism software to generate IC50. The results are shown in Table 12.

TABLE 11
Assay conditions for CYP inhibition in human liver microsomes:

| | Microsomes | Probe | Probe | Incubation | Positive | Negative |
|---|---|---|---|---|---|---|
| CYP | (mg/mL) | substrate | metabolite | (min) | control | control |
| 1A2 | 0.1 | Phenacetin | Acetaminophen | 10 | Fluvoxamine | Sulphaphenazole |
| | | (35 μM) | | | | |
| 2C9 | 0.1 | Diclofenac | 4′-OH- | 5 | Sulfaphenazole | Phenacetin |
| | | (10 μM) | diclofenac | | | |
| 2C19 | 0.25 | S-(+)- | 4′-OH- | 15 | Ticlopidine | Phenacetin |
| | | Mephenytoin | mephenytoin | | | |
| | | (30 μM) | | | | |
| 2D6 | 0.1 | Bufuralol | OH-bufuralol | 10 | Quinidine | Sulphaphenazole |
| | | (10 μM) | | | | |
| 3A4 | 0.1 | Midazolam | 1′-OH- | 5 | Ketoconazole | Sulphaphenazole |
| | | (M) | midazolam | | | |
| | | (3 μM) | | | | |
| 3A4 | 0.25 | Testosterone | 6β-OH- | 15 | Ketoconazole | Sulphaphenazole |
| | | (T) | testosterone | | | |
| | | (100 μM) | | | | |

TABLE 12
CYP inhibition of illustrative compounds of the invention

| Compound | % Inhibition (PIN) | | | | |
|---|---|---|---|---|---|
| number | 1A2 | 2C9 | 2C19 | 2D6 | 3A4 |
| A-10 | 22.97 | 46.15 | 38.60 | 14.79 | 54.18 |
| A-24 | 27.41 | 36.64 | 30.93 | 3.85 | 59.45 |
| A-31 | 6.09 | 73.02 | 73.05 | 23.15 | 94.62 |
| A-32 | 7.17 | 62.40 | 48.63 | 38.20 | 68.19 |
| A-82 | 9.89 | 32.06 | −4.12 | −2.54 | 50.58 |
| A-83 | 2.61 | 3.37 | 6.38 | −0.65 | 38.71 |
| A-84 | 5.60 | 56.18 | 18.85 | −7.67 | 49.14 |
| A-88 | 22.51 | 15.80 | 17.60 | 6.32 | 21.12 |
| A-90 | 10.34 | 28.44 | 27.88 | 1.84 | 55.31 |
| A-173 | 18.51 | 17.23 | 8.93 | 16.32 | 29.43 |
| A-174 | 22.68 | 20.95 | 26.49 | 9.35 | 36.05 |
| A-178 | 23.53 | 45.56 | 7.77 | 25.58 | 8.00 |
| A-217 | 16.00 | 51.00 | 39.00 | 68.00 | 73.00 (M)/ |
| | | | | | 25.00 (T) |
| A-223 | 16.00 | 43.00 | 25.00 | 15.00 | 82.00 (M)/ |
| | | | | | 28.00 (T) |

### In Vitro Permeability Assays

### PAMPA (Parallel Artificial Membrane Permeability Assay)

The Parallel Artificial Membrane Permeability Assay (PAMPA) is used as an in vitro model of passive, transcellular permeation. PAMPA eliminates the added complexities of active transport, allowing ranking compounds just based on a simple membrane permeability property. This simple assay also allows evaluation of permeability over a large pH range, which is valuable for a preliminary understanding of how orally delivered compounds might be absorbed across the entire gastrointestinal tract. Depending upon the types of lipids used and other experimental conditions, PAMPA may be designed to model absorption in gastrointestinal tract (PAMPA-GIT), blood-brain barrier penetration (PAMPA-BBB) or skin penetration (Skin PAMPA).

All steps of the PAMPA were carried out according to pION Inc. PAMPA Explorer™ Manual. The main principle of the assay is the incubation of compound in donor chamber (a well in Donor Plate) with aqueous buffer, which is separated from acceptor chamber (a well in Acceptor Plate) with another buffer by a phospholipid or hydrocarbon membrane fixed on a filter support. After the test, concentrations in the corresponding donor and acceptor wells are measured and permeability is calculated.

GIT model was simulated using GIT-0 phospholipid mix. Verapamil and quinidine (high permeability) and ranitidine (low permeability) were used as reference compounds. All compounds were tested in triplicates.

Solutions of the test and reference compounds were added into the Donor Plate wells in Prisma HT buffer (pH 7.4). Acceptor Sink Buffer was added into each well of the Acceptor Plate. Incubation was done at room temperature for 4 hours without stirring. After incubation, aliquots from both plates were transferred to optic UV-Vis plates and optic plates were read on microplate reader in absorbance mode in the range of 202-500 nm with 4 nm step. Compounds with low UV-Vis signal were detected by LC-MS/MS method (API 3000 (PE Sciex)). Both the positive and negative ion modes of the TurboIonSpray ion source were used. Acquisition and analysis of the data were performed using Analyst 1.5.2 software (PE Sciex). The apparent permeability coefficient was calculated according to the equation shown below:

$$log Papp = {log}_{10} ( - \frac{2.303 * {V}_{D}}{A \times ( t - {τ}_{ss} ) \times {ε}_{a}} \times {log}_{10} ( \frac{1}{1 - {R}_{M}} \times \frac{{C}_{don}}{{C}_{ref}} ) )$$

with:
V_{D}=volume of transport buffer in donor compartment
A=surface area of the lipids in the insert (effective growth area of the insert—0.3 sq.cm)
t=time of the assay, seconds
C_{don}=final concentration of test compound in the donor compartment
C_{ref}=starting concentration of test compound in the donor compartment
εₐ=apparent filter porosity, equals to 0.76

$${R}_{M} = membrane retention , calculated as {R}_{M} = ( 1 - \frac{{C}_{don} + {C}_{acc}}{{C}_{ref}} ) \times 100 \%$$

C_{acc}=final concentration of test compound in the donor compartment
τₛₛ=steady state lag time, estimated as (54R_{M}+1)×60 s

Values of C_{don}/C_{ref} and C_{acc}/C_{ref} are practically calculated using optical absorbance data. Mass retention by the lipid membrane is a normal condition in PAMPA assays for hydrophobic compounds but high levels of the mass retention makes compounds more permeable in vivo than it could be predicted from PAMPA assay. Mass retention was calculated as follows:

$$R = 100 \% - \frac{{[ drug ]}_{fd} + {[ drug ]}_{acc}}{{[ drug ]}_{d}} \times 100 \%$$

### With:

[drug]_{acc}=final OD of test compound in acceptor well
[drug]_{d}=starting OD of test compound in a donor well
[drug]_{fd}=final OD of test compound in a donor well

The results are shown in Table 13.

TABLE 13
PAMPA of illustrative compounds of the invention

| Compound | Permeability Papp, | Mass retention |
|---|---|---|
| number | Log10 (10⁻⁶ cm/s) | (%) |
| A-09 | <−7 | 10 |
| A-10 | <−7 | 8 |
| A-12 | <−7 | 7 |
| A-20 | <−7 | 28 |
| A-24 | <−7 | 24 |
| A-32 | −4.6 | 20 |
| A-51 | −4.2 | 14 |
| A-174 | −5.5 | 0 |

### Caco-2 Permeability Assay

Caco-2 cells were cultured in 75 cm² flasks to 80-90% confluence according to the ATCC and Millipore recommendations (Arena A. et al., 2003) in humidified atmosphere at 37° C. and 5% CO₂. Cells were detached with Trypsin/EDTA solution and resuspended in the cell culture medium to a final concentration of 2×10⁵ cells/ml. 500 μl of the cell suspension was added to each well of HTS 24-Multiwell Insert System and 1000 μl of prewarmed complete medium was added to each well of the feeder-plate. Caco-2 cells were incubated in Multiwell Insert System for 21 days before the transport experiments. The medium in filter plate and feeder tray was refreshed every other day. After 21 days of the cell growth, the integrity of the monolayer was verified by measuring the transepithelial electrical resistance (TEER) for every well using the Millicell-ERS system ohm meter. The final TEER values were within the range 150-600 Ω×cm² (Srinivasan B. et al., 2015) as required for the assay conditions. 24-well insert plate was removed from its feeder plate and placed in a new sterile 24-well transport analysis plate. The inserts were washed with PBS after medium aspiration. To determine the rate of compounds transport in apical (A)-to-basolateral (B) direction, 300 μL of the test compound dissolved in transport buffer at 10 μM (HBSS, 25 mM HEPES, pH=7.4) was added into the filter wells; 1000 μL of buffer (HBSS, 25 mM HEPES, pH=7.4) was added to transport analysis plate wells. Propranolol, Atenolol, Quinidine and Digoxin were used as reference compounds. The plates were incubated for 90 min at 37° C. under continuous shaking at 50 rpm. 75 μL aliquots were taken from the donor and receiver compartments for LC-MS/MS analysis. All samples were mixed with 2 volumes of acetonitrile followed by protein sedimentation by centrifuging at 10000 rpm for 10 minutes. Supernatants were analyzed using the HPLC system coupled with tandem mass spectrometer. The apparent permeability (Papp) was calculated for Caco-2 permeability assay using the following equation:

$${P}_{app} = \frac{{V}_{A}}{Area \times Time} \times \frac{{[ drug ]}_{acc}}{{[ drug ]}_{initial , d}}$$

### With:

V_{A}=volume of transport buffer in acceptor well
Area=surface area of the insert (equals to effective growth area of the insert −0.31 sq.cm)
Time=time of the assay
[drug]_{acc}=concentration of test compound in acceptor well
[drug]_{initial,d}=initial concentration of test compound in a donor well
Pₐₚₚ is expressed in 10⁻⁶ cm/sec.

The % recovery can be useful in interpreting the Caco-2 data. If the recovery is very low, this may indicate poor solubility, binding of the compound to the test plate materials, metabolism by the Caco-2 cells, or accumulation of the compound in the cell monolayer. The % recovery was calculated using the following equation:

$$\% recovery = \frac{{C}_{acc} \times {V}_{acc} + {C}_{d} \times {V}_{d}}{{C}_{initial , d} \times {V}_{d}} \times 100$$

### With:

V_{acc}=volume of compound solution in acceptor well (cm²)
V_{d}=volume of compound solution in donor well (cm²)
C_{acc}=concentration of test compound in acceptor well (μM)
C_{initial,d}=initial concentration of test compound in a donor well (μM)

The results are shown in Table 14.

TABLE 14
Caco-2 permeability of illustrative compounds of the invention

| | Permeability | Permeability | | |
|---|---|---|---|---|
| Compound | Papp (AB), | Papp (BA), | Efflux | Recovery |
| number | 10⁻⁶ cm/s | 10⁻⁶ cm/s | ratio | (%) |
| A-31 | 2.8 | | | 77 |
| A-90 | <3.4 | | | 91 |
| A-168 | 1.3 | 14.7 | 11.3 | 97 |
| A-178 | 1.2 | 8.6 | 7.2 | 94 |
| A-174 | 0.15 | 2.4 | 16.0 | 90 |

1.-5. (canceled)

6. A compound of Formula I: 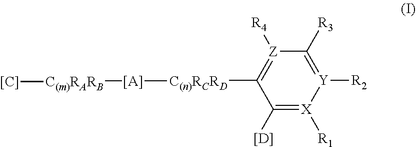
in which:
[C] is a monocyclic or polycyclic aromatic ring system which (i) contains at least one aromatic ring and (ii) is linked to the remainder of the compound of Formula I via a carbon atom of the at least one aromatic ring, the at least one aromatic ring having at least one heteroatom each independently and suitably chosen from N, S and O;
m is 1;
n is 0, and R_{C} and R_{D} are not present;
each of R_{A} and R_{B} is independently selected from the group consisting of hydrogen, methyl and/or trifluoromethyl, or R_{A} and R_{B}, together with the carbon atom to which they are bound, form a carbonyl (C═O) group;
[A] is:
(1) a ring system according to Scheme C: 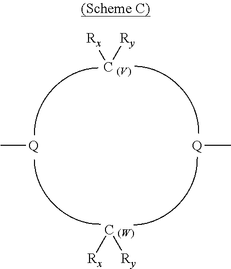
in which:
each Q is independently a carbon atom or a nitrogen atom;
v is 1, 2, 3 or 4 and w is 1, 2, 3 or 4, such that the sum of (v+w) is 3, 4, 5, 6, 7 or 8 and such that the difference (v-w) is 1, 0 or −1;
each Rₓ that is present and each R_{y} that is present is independently selected from the group consisting of hydrogen, methyl, ethyl, F, CF₃ and isopropyl; or
(2) Formula XCIX or Formula C: 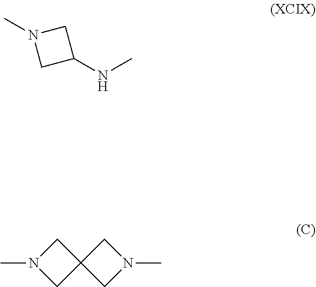
in which Formula XCIX or Formula C may be unsubstituted or substituted with one or more substituents selected from the group consisting of methyl, ethyl, F, CF₃, and isopropyl;
[D] is selected from the group consisting of COOH, CONHSO₂R or SO₂NHCOR, wherein R is —C₁-C₈ alkyl, CH₂(C₁-C₈ cycloalkyl), CH₂(heterocyclyl with 1-6 ring atoms), C₁-C₈ alkoxy or C₁-C₈ amine); tetrazole; and 4H-1,2,4-oxadiazol-5-one;
X and Y are each independently a nitrogen atom or a carbon atom, such that no more than one of X and Y is a nitrogen atom, And Z is a nitrogen atom or a carbon atom, wherein X, Y and Z are chosen such that the resulting ring structure is an aromatic ring when X is a nitrogen atom, R₁ is not present;
when Y is a nitrogen atom, R₂ is not present; and when Z is a nitrogen atom, R₄ is not present;
R₁, when present, is selected from the group consisting of H, F, Cl, Br, I, C₁-C₈ alkyl, C₃-C₄ cycloalkyl, CF₃, C₁-C₈ alkoxy, amine (—NH₂), C₁-C₂ substituted amine, and cyano;
R₂, when present, is selected from the group consisting of H, F, Cl, Br, I, C₁-C₈ alkyl, C₁-C₈ alkoxy, cycloalkyl, amine (—NH₂), and C₁-C₂ substituted amine;
R₃ is selected from the group consisting of H, F, Cl, Br, I, C₁₋₈ alkyl, fluoro-substituted methyl, C₁-C₈ alkoxy, —O—CF₃, —O—(CH₂)₂O—CH₃, —O—CH₂—CHF₂, C₃-C₆ cycloalkyl, —CH₂—(C₃-C₆ cycloalkyl), —O—CH₂—(C₃-C₆ cycloalkyl), —O—(C₃-C₆ cycloalkyl), —NH—(C₃-C₆ cycloalkyl), —N(C₁-C₃)—(C₃-C₆ cycloalkyl), —NH-heteroalkyl, —N(C₁-C₃)-heteroalkyl, 3-10 membered heterocyclyl, —CH₂-(heterocyclyl), —O—CH₂-(3-10 membered heterocyclyl), —O-(3-10 membered heterocyclyl), —NH-(3-membered heterocyclyl), —N(C₁-C₃)-(3-10 membered heterocyclyl), vinyl, methyl-substituted vinyl, allyl, methyl-substituted allyl, isobutenyl, methyl-substituted isobutenyl, and cyano, wherein a 3-10 membered heterocyclyl has 1-2 heteroatoms independently selected from N, O, and S; and
R₄, when present, is selected from the group consisting of H, F, Cl, Br, and I.

7. The compound of claim 6, which is a compound of Formula V:

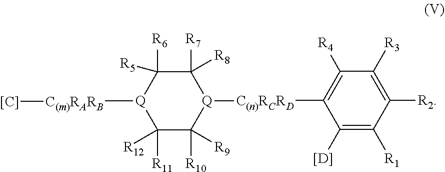

8. The compound of claim 7, wherein each Q is N.

9. The compound of claim 7, wherein [C] is monocyclic.

10. The compound of claim 6, which is a compound of Formula VI:

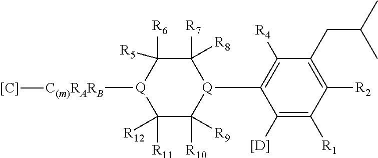

11. The compound of claim 7, wherein:
R_{A} is H;
R_{B} is H;
R₁ is selected from the group consisting of H, F, C₁-C₄ alkyl, C₃ cycloalkyl, and NH(C₁-C₂ alkyl);
R₂ is selected from the group consisting of H, F, C₁-C₄ alkyl, C₃ cycloalkyl, and C₁-C₄ alkoxy;
R₄ is H or F;
R₅ is H, methyl, ethyl, or isopropyl;
R₆ is H or methyl;
R₇ is H, methyl, or ethyl;
R₉ is H or methyl;
R₁₁ is H or methyl;
R₈, R₁₀, and R₁₂ are each H; and
D is tetrazole.

12. The compound of claim 6, selected from the group consisting of:
2-[[4-(1,3-benzothiazol-2-yl)piperazin-1-yl]methyl]benzoic acid;
2-[[4-(1,3-benzothiazol-2-yl)piperazin-1-yl]methyl]-N-ethylsulfonyl-benzamide;
2-[4-[[2-(2H-tetrazol-5-yl)phenyl]methyl]piperazin-1-yl]-1,3-benzothiazole;
2-[4-(1,3-benzothiazol-2-ylmethyl)piperazin-1-yl]-N-ethylsulfonyl-benzamide;
2-[[4-[2-(2H-tetrazol-5-yl)phenyl]piperazin-1-yl]methyl]-1,3-benzothiazole;
2-[[1-[2-(2H-tetrazol-5-yl)phenyl]-4-piperidyl]methyl]-1,3-benzothiazole;
6-nitro-2-[4-[[2-(2H-tetrazol-5-yl)phenyl]methyl]piperazin-1-yl]-1,3-benzothiazole;
ethyl N-[2-[4-[(1-methylbenzimidazol-2-yl)methyl]piperazin-1-yl]-4-(trifluoromethyl)phenyl]sulfonylcarbamate;
butyl N-[2-[4-[(1-methylbenzimidazol-2-yl)methyl]piperazin-1-yl]-4-(trifluoromethyl)phenyl]sulfonylcarbamate;
1-butyl-3-[2-[4-[(1-methylbenzimidazol-2-yl)methyl]piperazin-1-yl]-4-(trifluoromethyl)phenyl]sulfonyl-urea;
butyl N-[4-methyl-2-[4-[(1-methylbenzimidazol-2-yl)methyl]piperazin-1-yl]phenyl]sulfonylcarbamate;
1-butyl-3-[4-methyl-2-[4-[(1-methylbenzimidazol-2-yl)methyl]piperazin-1-yl]phenyl]sulfonyl-urea;
N-ethylsulfonyl-2-[4-[(1-methylbenzimidazol-2-yl)methyl]piperazin-1-yl]benzamide;
N-ethylsulfonyl-4-isopropoxy-2-[4-[(1-methylbenzimidazol-2-yl)methyl]piperazin-1-yl]benzamide;
1-methyl-2-[[4-[2-(2H-tetrazol-5-yl)phenyl]piperazin-1-yl]methyl]benzimidazole;
1-methyl-2-[[4-[5-methyl-2-(2H-tetrazol-5-yl)phenyl]piperazin-1-yl]methyl]benzimidazole;
1-methyl-2-[[4-[2-(2H-tetrazol-5-yl)-5-(trifluoromethyl)phenyl]piperazin-1-yl]methyl]benzimidazole;
1-methyl-2-[[4-[5-[(E)-prop-1-enyl]-2-(2H-tetrazol-5-yl)phenyl]piperazin-1-yl]methyl]benzimidazole;
1-methyl-2-[[4-[5-propyl-2-(2H-tetrazol-5-yl)phenyl]piperazin-1-yl]methyl]benzimidazole;
2-[[4-[5-isobutyl-2-(2H-tetrazol-5-yl)phenyl]piperazin-1-yl]methyl]-1-methyl-benzimidazole;
1-methyl-2-[[4-[5-(2-methylprop-1-enyl)-2-(2H-tetrazol-5-yl)phenyl]piperazin-1-yl]methyl]benzimidazole
2-[[4-[5-methoxy-2-(2H-tetrazol-5-yl)phenyl]piperazin-1-yl]methyl]-1-methyl-benzimidazole
2-[[4-[5-ethoxy-2-(2H-tetrazol-5-yl)phenyl]piperazin-1-yl]methyl]-1-methyl-benzimidazole
2-[[4-[5-isopropoxy-2-(2H-tetrazol-5-yl)phenyl]piperazin-1-yl]methyl]-1-methyl-benzimidazole
2-[[1-[5-isopropoxy-2-(2H-tetrazol-5-yl)phenyl]-4-piperidyl]methyl]-1-methyl-benzimidazole
2-[[4-[2-(2H-tetrazol-5-yl)phenyl]piperazin-1-yl]methyl]-3H-quinazolin-4-one
2-[[4-[5-methyl-2-(2H-tetrazol-5-yl)phenyl]piperazin-1-yl]methyl]-3H-quinazolin-4-one
2-[[4-[5-cyclopropyl-2-(2H-tetrazol-5-yl)phenyl]piperazin-1-yl]methyl]-3H-quinazolin-4-one
2-[[4-[2-(2H-tetrazol-5-yl)-5-(trifluoromethyl)phenyl]piperazin-1-yl]methyl]-3H-quinazolin-4-one
2-[[4-[5-(2-methylprop-1-enyl)-2-(2H-tetrazol-5-yl)phenyl]piperazin-1-yl]methyl]-3H-quinazolin-4-one
2-[[4-[5-isobutyl-2-(2H-tetrazol-5-yl)phenyl]piperazin-1-yl]methyl]-3H-quinazolin-4-one
2-[[4-[5-isopropoxy-2-(2H-tetrazol-5-yl)phenyl]piperazin-1-yl]methyl]-3H-quinazolin-4-one
2-[4-(1,3-benzothiazol-2-ylmethyl)piperazin-1-yl]-4-ethyl-N-ethylsulfonyl-benzamide
2-[4-(1,3-benzothiazol-2-ylmethyl)piperazin-1-yl]-N-ethylsulfonyl-4-isopropyl-benzamide
2-[4-(1,3-benzothiazol-2-ylmethyl)piperazin-1-yl]-4-cyclopropyl-N-ethylsulfonyl-benzamide
2-[4-(1,3-benzothiazol-2-ylmethyl)piperazin-1-yl]-N-ethylsulfonyl-4-isobutyl-benzamide
2-[4-(1,3-benzothiazol-2-ylmethyl)piperazin-1-yl]-N-ethylsulfonyl-4-methoxy-benzamide
2-[4-(1,3-benzothiazol-2-ylmethyl)piperazin-1-yl]-4-ethoxy-N-ethylsulfonyl-benzamide
2-[[4-(1,3-benzothiazol-2-yl)piperazin-1-yl]methyl]-N-ethylsulfonyl-4-isopropoxy-benzamide
2-[4-(1,3-benzothiazol-2-ylmethyl)piperazin-1-yl]-N-ethylsulfonyl-4-isopropoxy-benzamide
2-[4-(1,3-benzothiazol-2-ylmethyl)piperazin-1-yl]-4-(cyclopropoxy)-N-ethylsulfonyl-benzamide
2-[4-(1,3-benzothiazol-2-ylmethyl)piperazin-1-yl]-N-ethylsulfonyl-4-propoxy-benzamide
2-[4-(1,3-benzothiazol-2-ylmethyl)piperazin-1-yl]-N-ethylsulfonyl-4-isobutoxy-benzamide
2-[4-(1,3-benzothiazol-2-ylmethyl)piperazin-1-yl]-4-(ethylamino)-N-ethylsulfonyl-benzamide
2-[[4-[5-ethyl-2-(2H-tetrazol-5-yl)phenyl]piperazin-1-yl]methyl]-1,3-benzothiazole
2-[[4-[5-isopropyl-2-(2H-tetrazol-5-yl)phenyl]piperazin-1-yl]methyl]-1,3-benzothiazole
2-[[4-[5-cyclopropyl-2-(2H-tetrazol-5-yl)phenyl]piperazin-1-yl]methyl]-1,3-benzothiazole
2-[[4-[5-isobutyl-2-(2H-tetrazol-5-yl)phenyl]piperazin-1-yl]methyl]-1,3-benzothiazole
2-[4-[5-isobutyl-2-(2H-tetrazol-5-yl)phenyl]piperazin-1-yl]-1,3-benzothiazole
2-[4-[[5-isopropoxy-2-(2H-tetrazol-5-yl)phenyl]methyl]piperazin-1-yl]-1,3-benzothiazole
2-[[4-[5-isopropoxy-2-(2H-tetrazol-5-yl)phenyl]piperazin-1-yl]methyl]-1,3-benzothiazole
2-[[4-[5-(cyclopropoxy)-2-(2H-tetrazol-5-yl)phenyl]piperazin-1-yl]methyl]-1,3-benzothiazole
2-[[4-[5-isobutoxy-2-(2H-tetrazol-5-yl)phenyl]piperazin-1-yl]methyl]-1,3-benzothiazole
2-[[4-[5-(cyclopropylmethoxy)-2-(2H-tetrazol-5-yl)phenyl]piperazin-1-yl]methyl]-1,3-benzothiazole
2-[[4-[5-(2,2-difluoroethoxy)-2-(2H-tetrazol-5-yl)phenyl]piperazin-1-yl]methyl]-1,3-benzothiazole
2-[[4-[5-(2-methoxyethoxy)-2-(2H-tetrazol-5-yl)phenyl]piperazin-1-yl]methyl]-1,3-benzothiazole
2-[[4-[5-(cyclopentoxy)-2-(2H-tetrazol-5-yl)phenyl]piperazin-1-yl]methyl]-1,3-benzothiazole
2-[[4-[5-(oxetan-3-ylmethoxy)-2-(2H-tetrazol-5-yl)phenyl]piperazin-1-yl]methyl]-1,3-benzothiazole
3-[4-(1,3-benzothiazol-2-ylmethyl)piperazin-1-yl]-N-ethyl-4-(2H-tetrazol-5-yl)aniline
3-[4-(1,3-benzothiazol-2-ylmethyl)piperazin-1-yl]-N-isopropyl-4-(2H-tetrazol-5-yl)aniline
3-[4-(1,3-benzothiazol-2-ylmethyl)piperazin-1-yl]-N-cyclopropyl-4-(2H-tetrazol-5-yl)aniline hydrochloride
3-[4-(1,3-benzothiazol-2-ylmethyl)piperazin-1-yl]-N-(cyclopropylmethyl)-4-(2H-tetrazol-5-yl)aniline
3-[4-(1,3-benzothiazol-2-ylmethyl)piperazin-1-yl]-N-(2-methoxyethyl)-4-(2H-tetrazol-5-yl)aniline
3-[4-(1,3-benzothiazol-2-ylmethyl)piperazin-1-yl]-N-cyclopentyl-4-(2H-tetrazol-5-yl)aniline
2-[[4-[5-(1-piperidyl)-2-(2H-tetrazol-5-yl)phenyl]piperazin-1-yl]methyl]-1,3-benzothiazole
2-[[4-[5-(4-methylpiperazin-1-yl)-2-(2H-tetrazol-5-yl)phenyl]piperazin-1-yl]methyl]-1,3-benzothiazole
4-[3-[4-(1,3-benzothiazol-2-ylmethyl)piperazin-1-yl]-4-(2H-tetrazol-5-yl)phenyl]morpholine 3-[4-(1,3-benzothiazol-2-ylmethyl)piperazin-1-yl]-N-(3-methoxycyclobutyl)-4-(2H-tetrazol-5-yl)aniline
6-[[4-[5-isobutyl-2-(2H-tetrazol-5-yl)phenyl]piperazin-1-yl]methyl]-1-methyl-7H-pyrazolo[3,4-d]pyrimidin-4-one
2-[[4-[5-isobutyl-2-(2H-tetrazol-5-yl)phenyl]piperazin-1-yl]methyl]-7-methyl-pyrido[1,2-a]pyrimidin-4-one
2-[[4-[5-isobutyl-2-(2H-tetrazol-5-yl)phenyl]piperazin-1-yl]methyl]oxazolo[4,5-b]pyridine
2-[[4-[5-isobutyl-2-(2H-tetrazol-5-yl)phenyl]piperazin-1-yl]methyl]-8-methyl-pyrido[1,2-a]pyrimidin-4-one
2-[[4-[5-isobutyl-2-(2H-tetrazol-5-yl)phenyl]piperazin-1-yl]methyl]-6-methyl-pyrido[1,2-a]pyrimidin-4-one
5-[[4-[5-isobutyl-2-(2H-tetrazol-5-yl)phenyl]piperazin-1-yl]methyl]-1-methyl-pyrazolo[1,5-a]pyrimidin-7-one
7-[[4-[5-isobutyl-2-(2H-tetrazol-5-yl)phenyl]piperazin-1-yl]methyl]-3-methyl-thiazolo[3,2-a]pyrimidin-5-one
2-[[4-[5-isobutyl-2-(2H-tetrazol-5-yl)phenyl]piperazin-1-yl]methyl]pyrido[1,2-a]pyrimidin-4-one
3-[[4-[5-isobutyl-2-(2H-tetrazol-5-yl)phenyl]piperazin-1-yl]methyl]-1-methyl-quinolin-2-one
2-[[4-[5-isobutyl-2-(2H-tetrazol-5-yl)phenyl]piperazin-1-yl]methyl]-3-methyl-quinazolin-4-one
3-[[4-[5-isobutyl-2-(2H-tetrazol-5-yl)phenyl]piperazin-1-yl]methyl]-1-methyl-quinoxalin-2-one
3-[[4-[5-isobutyl-2-(2H-tetrazol-5-yl)phenyl]piperazin-1-yl]methyl]imidazo[1,2-a]pyridine
3-[[4-[5-isobutyl-2-(2H-tetrazol-5-yl)phenyl]piperazin-1-yl]methyl]-1-methyl-indazole
3-[[4-[5-isobutyl-2-(2H-tetrazol-5-yl)phenyl]piperazin-1-yl]methyl]-5-methyl-isoxazole
1-[5-isobutyl-2-(2H-tetrazol-5-yl)phenyl]-4-[(1-methylimidazol-2-yl)methyl]piperazine
1-[5-isobutyl-2-(2H-tetrazol-5-yl)phenyl]-4-[(2-methylpyrazol-3-yl)methyl]piperazine 1-[5-isobutyl-2-(2H-tetrazol-5-yl)phenyl]-4-[[5-(trifluoromethyl)-2-pyridyl]methyl]piperazine
3-[[4-[5-isobutyl-2-(2H-tetrazol-5-yl)phenyl]piperazin-1-yl]methyl]-5-methyl-1,2,4-oxadiazole
4-[[4-[5-isobutyl-2-(2H-tetrazol-5-yl)phenyl]piperazin-1-yl]methyl]-2-methyl-thiazole
2-[[4-[5-isobutyl-2-(2H-tetrazol-5-yl)phenyl]piperazin-1-yl]methyl]pyrimidine
5-[[4-[5-isobutyl-2-(2H-tetrazol-5-yl)phenyl]piperazin-1-yl]methyl]-3-methyl-isoxazole
3-[[4-[5-isobutyl-2-(2H-tetrazol-5-yl)phenyl]piperazin-1-yl]methyl]pyridazine
3-[[4-[5-cyclopropyl-2-(2H-tetrazol-5-yl)phenyl]piperazin-1-yl]methyl]pyridazine
2-[[4-[5-isobutyl-2-(2H-tetrazol-5-yl)phenyl]piperazin-1-yl]methyl]-5-methyl-1,3,4-oxadiazole
2-[[4-[5-isobutyl-2-(2H-tetrazol-5-yl)phenyl]piperazin-1-yl]methyl]pyrazine
1-[5-isobutyl-2-(2H-tetrazol-5-yl)phenyl]-4-[(4-methyl-1,2,4-triazol-3-yl)methyl]piperazine
6-[[4-[5-isobutyl-2-(2H-tetrazol-5-yl)phenyl]piperazin-1-yl]methyl]-2-methyl-1H-pyrimidin-4-one
5-[[4-[5-isobutyl-2-(2H-tetrazol-5-yl)phenyl]piperazin-1-yl]methyl]-3-methyl-1,2,4-oxadiazole
2-[[4-[5-isobutyl-2-(2H-tetrazol-5-yl)phenyl]piperazin-1-yl]methyl]thiazole
2-[[4-[5-isobutyl-2-(2H-tetrazol-5-yl)phenyl]piperazin-1-yl]methyl]oxazole
4-[[4-[5-isobutyl-2-(2H-tetrazol-5-yl)phenyl]piperazin-1-yl]methyl]-2-methyl-pyrimidine
2-[[4-[5-isobutyl-2-(2H-tetrazol-5-yl)phenyl]piperazin-1-yl]methyl]-6-methyl-1H-pyrimidin-4-one
4-[[4-[5-isobutyl-2-(2H-tetrazol-5-yl)phenyl]piperazin-1-yl]methyl]-3,5-dimethyl-isoxazole
5-cyclopropyl-3-[[4-[5-isobutyl-2-(2H-tetrazol-5-yl)phenyl]piperazin-1-yl]methyl]isoxazole
5-[[4-[5-isobutyl-2-(2H-tetrazol-5-yl)phenyl]piperazin-1-yl]methyl]-2-methyl-pyrimidine 1-[(2,5-dimethylpyrazol-3-yl)methyl]-4-[5-isobutyl-2-(2H-tetrazol-5-yl)phenyl]piperazine
1-[5-isobutyl-2-(2H-tetrazol-5-yl)phenyl]-4-[(3-methylimidazol-4-yl)methyl]piperazine
2-[[4-[5-isobutyl-2-(2H-tetrazol-5-yl)phenyl]piperazin-1-yl]methyl]-5-methyl-thiazole
6-[[4-[5-isobutyl-2-(2H-tetrazol-5-yl)phenyl]piperazin-1-yl]methyl]pyridine-2-carbonitrile
3-[[4-[5-isobutyl-2-(2H-tetrazol-5-yl)phenyl]piperazin-1-yl]methyl]-1,2,4-oxadiazole
1-[5-isobutyl-2-(2H-tetrazol-5-yl)phenyl]-4-[(1-methylimidazol-4-yl)methyl]piperazine
1-[5-isobutyl-2-(2H-tetrazol-5-yl)phenyl]-4-[(1-methylpyrazol-3-yl)methyl]piperazine
4-[[4-[5-isobutyl-2-(2H-tetrazol-5-yl)phenyl]piperazin-1-yl]methyl]-5-methyl-thiazole
3-[[4-[5-isobutyl-2-(2H-tetrazol-5-yl)phenyl]piperazin-1-yl]methyl]-5-methyl-pyridazine
3-[[4-[5-isobutyl-2-(2H-tetrazol-5-yl)phenyl]piperazin-1-yl]methyl]-6-methyl-pyridazine
1-[(2,3-dimethylimidazol-4-yl)methyl]-4-[5-isobutyl-2-(2H-tetrazol-5-yl)phenyl]piperazine
3-[[4-[5-isobutyl-2-(2H-tetrazol-5-yl)phenyl]piperazin-1-yl]methyl]-6-methoxy-pyridazine
2-[[4-[3-fluoro-5-methoxy-2-(2H-tetrazol-5-yl)phenyl]piperazin-1-yl]methyl]-1,3-benzothiazole
2-[[4-[4-fluoro-5-methoxy-2-(2H-tetrazol-5-yl)phenyl]piperazin-1-yl]methyl]-1,3-benzothiazole
2-[[4-[3-fluoro-5-isopropoxy-2-(2H-tetrazol-5-yl)phenyl]piperazin-1-yl]methyl]-1,3-benzothiazole
2-[[4-[4-ethyl-5-isopropoxy-2-(2H-tetrazol-5-yl)phenyl]piperazin-1-yl]methyl]-1,3-benzothiazole
2-[[4-[3-ethyl-5-isopropoxy-2-(2H-tetrazol-5-yl)phenyl]piperazin-1-yl]methyl]-1,3-benzothiazole 2-[[4-[4-cyclopropyl-5-isopropoxy-2-(2H-tetrazol-5-yl)phenyl]piperazin-1-yl]methyl]-1,3-benzothiazole
2-[[4-[3-cyclopropyl-5-isopropoxy-2-(2H-tetrazol-5-yl)phenyl]piperazin-1-yl]methyl]-1,3-benzothiazole
2-[[4-[4-ethoxy-5-isopropoxy-2-(2H-tetrazol-5-yl)phenyl]piperazin-1-yl]methyl]-1,3-benzothiazole
2-[[4-[5-cyclopropyl-4-fluoro-2-(2H-tetrazol-5-yl)phenyl]piperazin-1-yl]methyl]-1,3-benzothiazole
2-[[4-[5-cyclopropyl-3-fluoro-2-(2H-tetrazol-5-yl)phenyl]piperazin-1-yl]methyl]-1,3-benzothiazole
2-[[4-[5-cyclopropyl-4-methyl-2-(2H-tetrazol-5-yl)phenyl]piperazin-1-yl]methyl]-1,3-benzothiazole
2-[[4-[5-cyclopropyl-3-methyl-2-(2H-tetrazol-5-yl)phenyl]piperazin-1-yl]methyl]-1,3-benzothiazole
2-[[4-[5-cyclopropyl-3-ethyl-2-(2H-tetrazol-5-yl)phenyl]piperazin-1-yl]methyl]-1,3-benzothiazole
2-[[4-[3,5-dicyclopropyl-2-(2H-tetrazol-5-yl)phenyl]piperazin-1-yl]methyl]-1,3-benzothiazole
4-[4-(1,3-benzothiazol-2-ylmethyl)piperazin-1-yl]-2-cyclopropyl-N-ethyl-5-(2H-tetrazol-5-yl)aniline
N-[4-[4-(1,3-benzothiazol-2-ylmethyl)piperazin-1-yl]-2-cyclopropyl-5-(2H-tetrazol-5-yl)phenyl]acetamide
2-[[4-[3-fluoro-5-isobutyl-2-(2H-tetrazol-5-yl)phenyl]piperazin-1-yl]methyl]-1,3-benzothiazole
2-[[4-[4-fluoro-5-isobutyl-2-(2H-tetrazol-5-yl)phenyl]piperazin-1-yl]methyl]-1,3-benzothiazole
2-[[4-[5-isobutyl-4-methyl-2-(2H-tetrazol-5-yl)phenyl]piperazin-1-yl]methyl]-1,3-benzothiazole
2-[[4-[5-isobutyl-3-methyl-2-(2H-tetrazol-5-yl)phenyl]piperazin-1-yl]methyl]-1,3-benzothiazole
2-[[4-[3-ethyl-5-isobutyl-2-(2H-tetrazol-5-yl)phenyl]piperazin-1-yl]methyl]-1,3-benzothiazole 2-[[4-[3-cyclopropyl-5-isobutyl-2-(2H-tetrazol-5-yl)phenyl]piperazin-1-yl]methyl]-1,3-benzothiazole
4-[4-(1,3-benzothiazol-2-ylmethyl)piperazin-1-yl]-2-isobutyl-5-(2H-tetrazol-5-yl)aniline
4-[4-(1,3-benzothiazol-2-ylmethyl)piperazin-1-yl]-2-isobutyl-N-methyl-5-(2H-tetrazol-5-yl)aniline
4-[4-(1,3-benzothiazol-2-ylmethyl)piperazin-1-yl]-N-ethyl-2-isobutyl-5-(2H-tetrazol-5-yl)aniline
2-[[4-[5-ethyl-2-(2H-tetrazol-5-yl)-3-pyridyl]piperazin-1-yl]methyl]-1,3-benzothiazole
2-[[4-[6-ethyl-3-(2H-tetrazol-5-yl)-2-pyridyl]piperazin-1-yl]methyl]-1,3-benzothiazole
2-[[4-[5-cyclopropyl-2-(2H-tetrazol-5-yl)-3-pyridyl]piperazin-1-yl]methyl]-1,3-benzothiazole
2-[[4-[2-cyclopropyl-5-(2H-tetrazol-5-yl)-4-pyridyl]piperazin-1-yl]methyl]-1,3-benzothiazole
2-[[4-[6-cyclopropyl-3-(2H-tetrazol-5-yl)-2-pyridyl]piperazin-1-yl]methyl]-1,3-benzothiazole
2-[[4-[6-cyclopropyl-3-(2H-tetrazol-5-yl)pyrazin-2-yl]piperazin-1-yl]methyl]-1,3-benzothiazole
2-[[4-[5-isobutyl-2-(2H-tetrazol-5-yl)-3-pyridyl]piperazin-1-yl]methyl]-1,3-benzothiazole
2-[[4-[6-isobutyl-3-(2H-tetrazol-5-yl)-2-pyridyl]piperazin-1-yl]methyl]-1,3-benzothiazole
5-[4-(1,3-benzothiazol-2-ylmethyl)piperazin-1-yl]-N-(cyclopropylmethyl)-6-(2H-tetrazol-5-yl)pyridin-3-amine
2-[[4-[3-fluoro-5-methoxy-2-(2H-tetrazol-5-yl)phenyl]piperazin-1-yl]methyl]-3H-quinazolin-4-one
2-[[4-[3-fluoro-5-isopropoxy-2-(2H-tetrazol-5-yl)phenyl]piperazin-1-yl]methyl]-3H-quinazolin-4-one
2-[[4-[3-cyclopropyl-5-isopropoxy-2-(2H-tetrazol-5-yl)phenyl]piperazin-1-yl]methyl]-3H-quinazolin-4-one
2-[[4-[4-fluoro-5-isobutyl-2-(2H-tetrazol-5-yl)phenyl]piperazin-1-yl]methyl]-3H-quinazolin-4-one
2-[[4-[3-fluoro-5-isobutyl-2-(2H-tetrazol-5-yl)phenyl]piperazin-1-yl]methyl]-3H-quinazolin-4-one
3-[[4-[3-fluoro-5-methoxy-2-(2H-tetrazol-5-yl)phenyl]piperazin-1-yl]methyl]pyridazine
3-[[4-[3-fluoro-5-isopropoxy-2-(2H-tetrazol-5-yl)phenyl]piperazin-1-yl]methyl]pyridazine
3-[[4-[2-fluoro-3-isopropoxy-6-(2H-tetrazol-5-yl)phenyl]piperazin-1-yl]methyl]pyridazine
3-[[4-[4-ethyl-5-isopropoxy-2-(2H-tetrazol-5-yl)phenyl]piperazin-1-yl]methyl]pyridazine
3-[[4-[3-ethyl-5-isopropoxy-2-(2H-tetrazol-5-yl)phenyl]piperazin-1-yl]methyl]pyridazine
3-[[4-[4-cyclopropyl-5-isopropoxy-2-(2H-tetrazol-5-yl)phenyl]piperazin-1-yl]methyl]pyridazine
3-[[4-[3-cyclopropyl-5-isopropoxy-2-(2H-tetrazol-5-yl)phenyl]piperazin-1-yl]methyl]pyridazine
3-[[4-[4-ethoxy-5-isopropoxy-2-(2H-tetrazol-5-yl)phenyl]piperazin-1-yl]methyl]pyridazine
3-[[4-[4-fluoro-5-isobutyl-2-(2H-tetrazol-5-yl)phenyl]piperazin-1-yl]methyl]pyridazine
3-[[4-[3-fluoro-5-isobutyl-2-(2H-tetrazol-5-yl)phenyl]piperazin-1-yl]methyl]pyridazine
3-[[4-[2-fluoro-3-isobutyl-6-(2H-tetrazol-5-yl)phenyl]piperazin-1-yl]methyl]pyridazine
3-[[4-[5-isobutyl-4-methyl-2-(2H-tetrazol-5-yl)phenyl]piperazin-1-yl]methyl]pyridazine
3-[[4-[5-isobutyl-3-methyl-2-(2H-tetrazol-5-yl)phenyl]piperazin-1-yl]methyl]pyridazine
3-[[4-[3-ethyl-5-isobutyl-2-(2H-tetrazol-5-yl)phenyl]piperazin-1-yl]methyl]pyridazine
3-[[4-[3-cyclopropyl-5-isobutyl-2-(2H-tetrazol-5-yl)phenyl]piperazin-1-yl]methyl]pyridazine 2-isobutyl-N-methyl-4-[4-(pyridazin-3-ylmethyl)piperazin-1-yl]-5-(2H-tetrazol-5-yl)aniline
5-isobutyl-N-methyl-3-[4-(pyridazin-3-ylmethyl)piperazin-1-yl]-2-(2H-tetrazol-5-yl)aniline
N-ethyl-2-isobutyl-4-[4-(pyridazin-3-ylmethyl)piperazin-1-yl]-5-(2H-tetrazol-5-yl)aniline
3-[[4-[3-fluoro-5-isobutyl-2-(2H-tetrazol-5-yl)phenyl]-2-methyl-piperazin-1-yl]methyl]pyridazine
3-[[(2S)-4-[3-fluoro-5-isobutyl-2-(2H-tetrazol-5-yl)phenyl]-2-methyl-piperazin-1-yl]methyl]pyridazine
3-[[(2R)-4-[3-fluoro-5-isobutyl-2-(2H-tetrazol-5-yl)phenyl]-2-methyl-piperazin-1-yl]methyl]pyridazine
3-[[(2S)-4-[2-fluoro-3-isobutyl-6-(2H-tetrazol-5-yl)phenyl]-2-methyl-piperazin-1-yl]methyl]pyridazine
3-[[(2S)-4-[5-isobutyl-2-(2H-tetrazol-5-yl)-3-pyridyl]-2-methyl-piperazin-1-yl]methyl]pyridazine
3-[[2-ethyl-4-[3-fluoro-5-isobutyl-2-(2H-tetrazol-5-yl)phenyl]piperazin-1-yl]methyl]pyridazine
3-[[4-[3-fluoro-5-isobutyl-2-(2H-tetrazol-5-yl)phenyl]-2-isopropyl-piperazin-1-yl]methyl]pyridazine
3-[[4-[3-fluoro-5-isobutyl-2-(2H-tetrazol-5-yl)phenyl]-3-methyl-piperazin-1-yl]methyl]pyridazine
3-[[3-ethyl-4-[3-fluoro-5-isobutyl-2-(2H-tetrazol-5-yl)phenyl]piperazin-1-yl]methyl]pyridazine
3-[[4-[3-fluoro-5-isobutyl-2-(2H-tetrazol-5-yl)phenyl]-2,2-dimethyl-piperazin-1-yl]methyl]pyridazine
3-[[4-[3-fluoro-5-isobutyl-2-(2H-tetrazol-5-yl)phenyl]-2,5-dimethyl-piperazin-1-yl]methyl]pyridazine
3-[[4-[3-fluoro-5-isobutyl-2-(2H-tetrazol-5-yl)phenyl]-2,6-dimethyl-piperazin-1-yl]methyl]pyridazine
3-[[(2R,6R)-4-[3-fluoro-5-isobutyl-2-(2H-tetrazol-5-yl)phenyl]-2,6-dimethyl-piperazin-1-yl]methyl]pyridazine 3-[[(2S,6R)-4-[3-fluoro-5-isobutyl-2-(2H-tetrazol-5-yl)phenyl]-2,6-dimethyl-piperazin-1-yl]methyl]pyridazine
3-[[(2S,6S)-4-[3-fluoro-5-isobutyl-2-(2H-tetrazol-5-yl)phenyl]-2,6-dimethyl-piperazin-1-yl]methyl]pyridazine
3-[[4-[3-fluoro-5-isobutyl-2-(2H-tetrazol-5-yl)phenyl]-2,3-dimethyl-piperazin-1-yl]methyl]pyridazine
2-[3-fluoro-5-isobutyl-2-(2H-tetrazol-5-yl)phenyl]-5-(pyridazin-3-ylmethyl)-2,5-diazabicyclo[2.2.1]heptane
3-[3-fluoro-5-isobutyl-2-(2H-tetrazol-5-yl)phenyl]-8-(pyridazin-3-ylmethyl)-3, 8-diazabicyclo[3.2.1]octane
1-[(4-chloro-2-pyridyl)methyl]-4-[3-fluoro-5-isobutyl-2-(2H-tetrazol-5-yl)phenyl]piperazine
1-[(5-chloro-2-pyridyl)methyl]-4-[3-fluoro-5-isobutyl-2-(2H-tetrazol-5-yl)phenyl]piperazine
1-[(3-chloro-2-pyridyl)methyl]-4-[3-fluoro-5-isobutyl-2-(2H-tetrazol-5-yl)phenyl]piperazine
1-[3-fluoro-5-isobutyl-2-(2H-tetrazol-5-yl)phenyl]-4-[(5-methoxy-2-pyridyl)methyl]piperazine
1-[3-fluoro-5-isobutyl-2-(2H-tetrazol-5-yl)phenyl]-4-[(5-fluoro-2-pyridyl)methyl]piperazine
1-[3-fluoro-5-isobutyl-2-(2H-tetrazol-5-yl)phenyl]-4-[(3-fluoro-2-pyridyl)methyl]piperazine
1-[(3,5-dimethyl-2-pyridyl)methyl]-4-[3-fluoro-5-isobutyl-2-(2H-tetrazol-5-yl)phenyl]piperazine
1-[3-fluoro-5-isobutyl-2-(2H-tetrazol-5-yl)phenyl]-4-[(3-methoxy-2-pyridyl)methyl]piperazine
1-[3-fluoro-5-isobutyl-2-(2H-tetrazol-5-yl)phenyl]-4-[(4-methoxy-2-pyridyl)methyl]piperazine
1-[3-fluoro-5-isobutyl-2-(2H-tetrazol-5-yl)phenyl]-4-[(4-methoxy-3,5-dimethyl-2-pyridyl)methyl]piperazine
1-[3-fluoro-5-isobutyl-2-(2H-tetrazol-5-yl)phenyl]-4-[(5-methyl-2-pyridyl)methyl]piperazine 1-[3-fluoro-5-isobutyl-2-(2H-tetrazol-5-yl)phenyl]-4-[[4-(trifluoromethyl)-2-pyridyl]methyl]piperazine
1-[3-fluoro-5-isobutyl-2-(2H-tetrazol-5-yl)phenyl]-4-[(3-methyl-2-pyridyl)methyl]piperazine
1-[3-fluoro-5-isobutyl-2-(2H-tetrazol-5-yl)phenyl]-4-[(4-methyl-2-pyridyl)methyl]piperazine
1-[(5-chloro-3-fluoro-2-pyridyl)methyl]-4-[3-fluoro-5-isobutyl-2-(2H-tetrazol-5-yl)phenyl]piperazine
1-[(3-chloro-5-fluoro-2-pyridyl)methyl]-4-[3-fluoro-5-isobutyl-2-(2H-tetrazol-5-yl)phenyl]piperazine
1-[(4,5-dimethoxy-2-pyridyl)methyl]-4-[3-fluoro-5-isobutyl-2-(2H-tetrazol-5-yl)phenyl]piperazine
1-[3-fluoro-5-isobutyl-2-(2H-tetrazol-5-yl)phenyl]-4-[(4-methoxy-3-methyl-2-pyridyl)methyl]piperazine
6-[[4-[3-fluoro-5-isobutyl-2-(2H-tetrazol-5-yl)phenyl]piperazin-1-yl]methyl]pyridine-3-carbonitrile
1-[(4-chloro-5-fluoro-2-pyridyl)methyl]-4-[3-fluoro-5-isobutyl-2-(2H-tetrazol-5-yl)phenyl]piperazine
1-[(3,5-difluoro-2-pyridyl)methyl]-4-[3-fluoro-5-isobutyl-2-(2H-tetrazol-5-yl)phenyl]piperazine
3-[1-[4-[5-isobutyl-2-(2H-tetrazol-5-yl)phenyl]piperazin-1-yl]ethyl]pyridazine
[4-[3-fluoro-5-isobutyl-2-(2H-tetrazol-5-yl)phenyl]piperazin-1-yl]-pyridazin-3-yl-methanone
1-[3-fluoro-5-isobutyl-2-(2H-tetrazol-5-yl)phenyl]-4-[1-(2-pyridyl)ethyl]piperazine
[4-[3-fluoro-5-isobutyl-2-(2H-tetrazol-5-yl)phenyl]piperazin-1-yl]-(2-pyridyl)methanone
[(2S)-4-[3-fluoro-5-isobutyl-2-(2H-tetrazol-5-yl)phenyl]-2-methyl-piperazin-1-yl]-(2-pyridyl)methanone
(2S)-4-[3-fluoro-5-isobutyl-2-(2H-tetrazol-5-yl)phenyl]-2-methyl-1-(2-pyridylmethyl)piperazine
(2S)-4-[3-fluoro-5-isobutyl-2-(2H-tetrazol-5-yl)phenyl]-2-methyl-1-[1-(2-pyridyl)ethyl]piperazine 2-[[4-[2-fluoro-3-isobutyl-6-(2H-tetrazol-5-yl)phenyl]piperazin-1-yl]methyl]-5-methyl-thiazole
2-[[4-[3-fluoro-5-isobutyl-2-(2H-tetrazol-5-yl)phenyl]piperazin-1-yl]methyl]-5-methyl-thiazole
2-[[4-[5-isobutyl-2-(2H-tetrazol-5-yl)-3-pyridyl]piperazin-1-yl]methyl]-5-methyl-thiazole
2-[[4-[3-fluoro-5-isobutyl-2-(2H-tetrazol-5-yl)phenyl]-2-methyl-piperazin-1-yl]methyl]-5-methyl-thiazole
2-[[(2S)-4-[3-fluoro-5-isobutyl-2-(2H-tetrazol-5-yl)phenyl]-2-methyl-piperazin-1-yl]methyl]-5-methyl-thiazole
2-[[4-[3-fluoro-5-isobutyl-2-(2H-tetrazol-5-yl)phenyl]-2,6-dimethyl-piperazin-1-yl]methyl]-5-methyl-thiazole
2-[1-[4-[3-fluoro-5-isobutyl-2-(2H-tetrazol-5-yl)phenyl]piperazin-1-yl]ethyl]-5-methyl-thiazole
2-[1-[(2S)-4-[3-fluoro-5-isobutyl-2-(2H-tetrazol-5-yl)phenyl]-2-methyl-piperazin-1-yl]ethyl]-5-methyl-thiazole
(2S)-4-[3-fluoro-5-isobutyl-2-(2H-tetrazol-5-yl)phenyl]-1-[(4-methoxy-2-pyridyl)methyl]-2-methyl-piperazine
(2S)-4-[3-fluoro-5-isobutyl-2-(2H-tetrazol-5-yl)phenyl]-2-methyl-1-[(3-methyl-2-pyridyl)methyl]piperazine
(2S)-4-[3-fluoro-5-isobutyl-2-(2H-tetrazol-5-yl)phenyl]-2-methyl-I-[(4-methyl-2-pyridyl)methyl]piperazine
1-[5-isobutyl-2-(2H-tetrazol-5-yl)phenyl]-4-(pyridazin-3-ylmethyl)-1,4-diazepane
1-[3-fluoro-5-isobutyl-2-(2H-tetrazol-5-yl)phenyl]-4-(pyridazin-3-ylmethyl)-1,4-diazepane
5-[5-isobutyl-2-(2H-tetrazol-5-yl)phenyl]-2-(pyridazin-3-ylmethyl)-1,3,3a,4,6,6a-hexahydropyrrolo[3,4-c]pyrrole
5-[3-fluoro-5-isobutyl-2-(2H-tetrazol-5-yl)phenyl]-2-(pyridazin-3-ylmethyl)-1,3,3a,4,6,6a-hexahydropyrrolo[3,4-c]pyrrole
3-[[4-[5-isobutyl-2-(2H-tetrazol-5-yl)phenyl]-1-piperidyl]methyl]pyridazine
3-[[3-[5-isobutyl-2-(2H-tetrazol-5-yl)phenyl]pyrrolidin-1-yl]methyl]pyridazine
1-(5-isobutyl-2-(2H-tetrazol-5-yl)phenyl)-N-(pyridazin-3-ylmethyl)azetidin-3-amine
2-((1-(3-fluoro-5-isobutyl-2-(2H-tetrazol-5-yl)phenyl)piperidin-4-yl)methyl)pyridine
3-[[4-[[5-isobutyl-2-(2H-tetrazol-5-yl)phenyl]methyl]piperazin-1-yl]methyl]pyridazine
(5-isobutyl-2-(2H-tetrazol-5-yl)phenyl)(4-(pyridazin-3-ylmethyl)piperazin-1-yl)methanone
2-ethyl-3-[[1-[2-(2H-tetrazol-5-yl)phenyl]-4-piperidyl]methyl]quinazolin-4-one
2-[4-[(2-ethyl-4-oxo-quinazolin-3-yl)methyl]-1-piperidyl]-N-ethylsulfonyl-benzamide
2-ethyl-6-nitro-3-[[1-[2-(2H-tetrazol-5-yl)phenyl]-4-piperidyl]methyl]quinazolin-4-one
N-[2-ethyl-4-oxo-3-[[1-[2-(2H-tetrazol-5-yl)phenyl]-4-piperidyl]methyl]quinazolin-6-yl]-N-propyl-thiophene-2-carboxamide
N-[2-ethyl-4-oxo-3-[[1-[2-(2H-tetrazol-5-yl)phenyl]-4-piperidyl]methyl]quinazolin-6-yl]-N-(3-pyridylmethyl)thiophene-2-carboxamide
N-[2-ethyl-4-oxo-3-[[1-[2-(2H-tetrazol-5-yl)phenyl]-4-piperidyl]methyl]quinazolin-6-yl]benzamide
N-benzyl-N-[2-ethyl-4-oxo-3-[[1-[2-(2H-tetrazol-5-yl)phenyl]-4-piperidyl]methyl]quinazolin-6-yl]thiophene-2-carboxamide
N-[2-ethyl-4-oxo-3-[[1-[2-(2H-tetrazol-5-yl)phenyl]-4-piperidyl]methyl]quinazolin-6-yl]cyclopropanecarboxamide
N-[(2-chlorophenyl)methyl]-N-[2-ethyl-4-oxo-3-[[1-[2-(2H-tetrazol-5-yl)phenyl]-4-piperidyl]methyl]quinazolin-6-yl]thiophene-2-carboxamide
2-phenyl-3-[[1-[2-(1H-tetrazol-5-yl)phenyl]-4-piperidyl]methyl]quinazolin-4-one
3-[[(2S)-4-[5-(1,2-dideuterio-2-methyl-propyl)-3-f(luoro-2-(2H-tetrazol-5-yl)phenyl]-2-methyl-piperazin-1-yl]methyl]pyridazine
2,2,3,3,5,6,6-heptadeuterio-1-[3-fluoro-5-isobutyl-2-(2H-tetrazol-5-yl)phenyl]-4-(pyridazin-3-ylmethyl)-5-(trideuteriomethyl)piperazine.

13. The compound of claim 7, selected from the group consisting of:
2-[[4-[5-cyclopropyl-3-fluoro-2-(2H-tetrazol-5-yl)phenyl]piperazin-1-yl]methyl]-1,3-benzothiazole;
3-[[4-[5-isobutyl-2-(2H-tetrazol-5-yl)phenyl]piperazin-1-yl]methyl]imidazo[1,2-a]pyridine;
2-[[4-[3-fluoro-5-isobutyl-2-(2H-tetrazol-5-yl)phenyl]piperazin-1-yl]methyl]-1,3-benzothiazole;
2-[4-(1,3-benzothiazol-2-ylmethyl)piperazin-1-yl]-N-ethylsulfonyl-4-isopropyl-benzamide;
2-[[4-[3-fluoro-5-isobutyl-2-(2H-tetrazol-5-yl)phenyl]piperazin-1-yl]methyl]-3H-quinazolin-4-one;
2-[[4-[3-cyclopropyl-5-isopropoxy-2-(2H-tetrazol-5-yl)phenyl]piperazin-1-yl]methyl]-1,3-benzothiazole;
2-[[4-[5-isobutyl-2-(2H-tetrazol-5-yl)phenyl]piperazin-1-yl]methyl]-5-methyl-thiazole;
2-[[4-[3,5-dicyclopropyl-2-(2H-tetrazol-5-yl)phenyl]piperazin-1-yl]methyl]-1,3-benzothiazole;
3-[[4-[3-fluoro-5-isobutyl-2-(2H-tetrazol-5-yl)phenyl]-3-methyl-piperazin-1-yl]methyl]pyridazine;
3-[[4-[3-fluoro-5-isobutyl-2-(2H-tetrazol-5-yl)phenyl]-2-methyl-piperazin-1-yl]methyl]pyridazine;
3-[[2-ethyl-4-[3-fluoro-5-isobutyl-2-(2H-tetrazol-5-yl)phenyl]piperazin-1-yl]methyl]pyridazine;
2-[[4-[3-ethyl-5-isobutyl-2-(2H-tetrazol-5-yl)phenyl]piperazin-1-yl]methyl]-1,3-benzothiazole;
2-[[4-[5-cyclopropyl-3-methyl-2-(2H-tetrazol-5-yl)phenyl]piperazin-1-yl]methyl]-1,3-benzothiazole;
3-[[4-[3-cyclopropyl-5-isobutyl-2-(2H-tetrazol-5-yl)phenyl]piperazin-1-yl]methyl]pyridazine;
3-[[4-[3-ethyl-5-isobutyl-2-(2H-tetrazol-5-yl)phenyl]piperazin-1-yl]methyl]pyridazine;
5-isobutyl-N-methyl-3-[4-(pyridazin-3-ylmethyl)piperazin-1-yl]-2-(2H-tetrazol-5-yl)aniline;
2-[[4-[3-cyclopropyl-5-isobutyl-2-(2H-tetrazol-5-yl)phenyl]piperazin-1-yl]methyl]-1,3-benzothiazole;
2-[[4-[5-isobutyl-3-methyl-2-(2H-tetrazol-5-yl)phenyl]piperazin-1-yl]methyl]-1,3-benzothiazole;
3-[[3-ethyl-4-[3-fluoro-5-isobutyl-2-(2H-tetrazol-5-yl)phenyl]piperazin-1-yl]methyl]pyridazine;
3-[[4-[5-isobutyl-3-methyl-2-(2H-tetrazol-5-yl)phenyl]piperazin-1-yl]methyl]pyridazine;
3-[[4-[3-fluoro-5-isobutyl-2-(2H-tetrazol-5-yl)phenyl]-2,5-dimethyl-piperazin-1-yl]methyl]pyridazine;
3-[[4-[3-fluoro-5-isobutyl-2-(2H-tetrazol-5-yl)phenyl]-2,2-dimethyl-piperazin-1-yl]methyl]pyridazine;
3-[1-[4-[5-isobutyl-2-(2H-tetrazol-5-yl)phenyl]piperazin-1-yl]ethyl]pyridazine;
3-[[4-[3-fluoro-5-isobutyl-2-(2H-tetrazol-5-yl)phenyl]-2,6-dimethyl-piperazin-1-yl]methyl]pyridazine;
3-[[4-[3-fluoro-5-isobutyl-2-(2H-tetrazol-5-yl)phenyl]-2,3-dimethyl-piperazin-1-yl]methyl]pyridazine;
3-[[(2S)-4-[3-fluoro-5-isobutyl-2-(2H-tetrazol-5-yl)phenyl]-2-methyl-piperazin-1-yl]methyl]pyridazine;
3-[[(2R)-4-[3-fluoro-5-isobutyl-2-(2H-tetrazol-5-yl)phenyl]-2-methyl-piperazin-1-yl]methyl]pyridazine;
1-[(4-chloro-2-pyridyl)methyl]-4-[3-fluoro-5-isobutyl-2-(2H-tetrazol-5-yl)phenyl]piperazine;
1-[(3-chloro-2-pyridyl)methyl]-4-[3-fluoro-5-isobutyl-2-(2H-tetrazol-5-yl)phenyl]piperazine;
1-[3-fluoro-5-isobutyl-2-(2H-tetrazol-5-yl)phenyl]-4-[(4-methoxy-2-pyridyl)methyl]piperazine;
1-[(3,5-dimethyl-2-pyridyl)methyl]-4-[3-fluoro-5-isobutyl-2-(2H-tetrazol-5-yl)phenyl]piperazine;
[4-[3-fluoro-5-isobutyl-2-(2H-tetrazol-5-yl)phenyl]piperazin-1-yl]-pyridazin-3-yl-methanone;
2-[[4-[3-fluoro-5-isobutyl-2-(2H-tetrazol-5-yl)phenyl]piperazin-1-yl]methyl]-5-methyl-thiazole;
2-[1-[4-[3-fluoro-5-isobutyl-2-(2H-tetrazol-5-yl)phenyl]piperazin-1-yl]ethyl]-5-methyl-thiazole;
2-[[4-[3-fluoro-5-isobutyl-2-(2H-tetrazol-5-yl)phenyl]-2-methyl-piperazin-1-yl]methyl]-5-methyl-thiazole;
2-[[(2S)-4-[3-fluoro-5-isobutyl-2-(2H-tetrazol-5-yl)phenyl]-2-methyl-piperazin-1-yl]methyl]-5-methyl-thiazole;
1-[3-fluoro-5-isobutyl-2-(2H-tetrazol-5-yl)phenyl]-4-[(3-methyl-2-pyridyl)methyl]piperazine;
1-[3-fluoro-5-isobutyl-2-(2H-tetrazol-5-yl)phenyl]-4-[(4-methyl-2-pyridyl)methyl]piperazine;
1-[(5-chloro-3-fluoro-2-pyridyl)methyl]-4-[3-fluoro-5-isobutyl-2-(2H-tetrazol-5-yl)phenyl]piperazine;
2-[[4-[3-fluoro-5-isobutyl-2-(2H-tetrazol-5-yl)phenyl]-2,6-dimethyl-piperazin-1-yl]methyl]-5-methyl-thiazole;
2-[i-[(2S)-4-[3-fluoro-5-isobutyl-2-(2H-tetrazol-5-yl)phenyl]-2-methyl-piperazin-1-yl]ethyl]-5-methyl-thiazole;
3-[[(2S)-4-[2-fluoro-3-isobutyl-6-(2H-tetrazol-5-yl)phenyl]-2-methyl-piperazin-1-yl]methyl]pyridazine;
3-[[(2R,6R)-4-[3-fluoro-5-isobutyl-2-(2H-tetrazol-5-yl)phenyl]-2,6-dimethyl-piperazin-1-yl]methyl]pyridazine;
3-[[(2S,6R)-4-[3-fluoro-5-isobutyl-2-(2H-tetrazol-5-yl)phenyl]-2,6-dimethyl-piperazin-1-yl]methyl]pyridazine;
3-[[(2S,6S)-4-[3-fluoro-5-isobutyl-2-(2H-tetrazol-5-yl)phenyl]-2,6-dimethyl-piperazin-1-yl]methyl]pyridazine;
(2S)-4-[3-fluoro-5-isobutyl-2-(2H-tetrazol-5-yl)phenyl]-1-[(4-methoxy-2-pyridyl)methyl]-2-methyl-piperazine;
(2S)-4-[3-fluoro-5-isobutyl-2-(2H-tetrazol-5-yl)phenyl]-2-methyl-1-[(3-methyl-2-pyridyl)methyl]piperazine;
(2S)-4-[3-fluoro-5-isobutyl-2-(2H-tetrazol-5-yl)phenyl]-2-methyl-1-[(4-methyl-2-pyridyl)methyl]piperazine;
1-[3-fluoro-5-isobutyl-2-(2H-tetrazol-5-yl)phenyl]-4-(pyridazin-3-ylmethyl)-1,4-diazepane;
[4-[3-fluoro-5-isobutyl-2-(2H-tetrazol-5-yl)phenyl]piperazin-1-yl]-(2-pyridyl)methanone;
(2S)-4-[3-fluoro-5-isobutyl-2-(2H-tetrazol-5-yl)phenyl]-2-methyl-1-(2-pyridylmethyl)piperazine; and
(2S)-4-[3-fluoro-5-isobutyl-2-(2H-tetrazol-5-yl)phenyl]-2-methyl-1-[1-(2-pyridyl)ethyl]piperazine;
or a pharmaceutically acceptable salt thereof.

14. The compound of claim 11, which is: 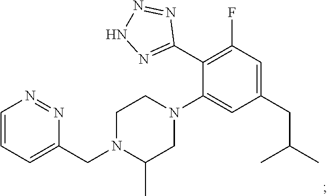 [SMILES: CC(C)Cc1cc(F)c(-c2nn[nH]n2)c(N2CCN(Cc3cccnn3)C(C)C2)c1]
or a pharmaceutically acceptable salt thereof.

15. The compound of claim 14, which is: 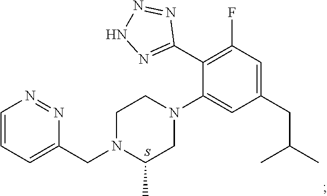
or a pharmaceutically acceptable salt thereof.

16. The compound of claim 14, which is: 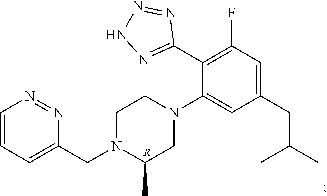
or a pharmaceutically acceptable salt thereof.

17. The compound of claim 6, selected from the group consisting of:
2-[[4-(1,3-benzothiazol-2-yl)piperazin-1-yl]methyl]benzoic acid;
2-[[4-(1,3-benzothiazol-2-yl)piperazin-1-yl]methyl]-N-ethylsulfonyl-benzamide;
2-[4-[[2-(2H-tetrazol-5-yl)phenyl]methyl]piperazin-1-yl]-1,3-benzothiazole;
2-[4-(1,3-benzothiazol-2-ylmethyl)piperazin-1-yl]-N-ethylsulfonyl-benzamide;
2-[[4-[2-(2H-tetrazol-5-yl)phenyl]piperazin-1-yl]methyl]-1,3-benzothiazole;
2-[[1-[2-(2H-tetrazol-5-yl)phenyl]-4-piperidyl]methyl]-1,3-benzothiazole;
6-nitro-2-[4-[[2-(2H-tetrazol-5-yl)phenyl]methyl]piperazin-1-yl]-1,3-benzothiazole;
ethyl N-[2-[4-[(1-methylbenzimidazol-2-yl)methyl]piperazin-1-yl]-4-(trifluoromethyl)phenyl]sulfonylcarbamate;
butyl N-[2-[4-[(1-methylbenzimidazol-2-yl)methyl]piperazin-1-yl]-4-(trifluoromethyl)phenyl]sulfonylcarbamate;
1-butyl-3-[2-[4-[(1-methylbenzimidazol-2-yl)methyl]piperazin-1-yl]-4-(trifluoromethyl)phenyl]sulfonyl-urea;
butyl N-[4-methyl-2-[4-[(1-methylbenzimidazol-2-yl)methyl]piperazin-1-yl]phenyl]sulfonylcarbamate;
1-butyl-3-[4-methyl-2-[4-[(1-methylbenzimidazol-2-yl)methyl]piperazin-1-yl]phenyl]sulfonyl-urea;
N-ethylsulfonyl-2-[4-[(1-methylbenzimidazol-2-yl)methyl]piperazin-1-yl]benzamide;
N-ethylsulfonyl-4-isopropoxy-2-[4-[(1-methylbenzimidazol-2-yl)methyl]piperazin-1-yl]benzamide;
1-methyl-2-[[4-[2-(2H-tetrazol-5-yl)phenyl]piperazin-1-yl]methyl]benzimidazole;
1-methyl-2-[[4-[5-methyl-2-(2H-tetrazol-5-yl)phenyl]piperazin-1-yl]methyl]benzimidazole;
1-methyl-2-[[4-[2-(2H-tetrazol-5-yl)-5-(trifluoromethyl)phenyl]piperazin-1-yl]methyl]benzimidazole;
1-methyl-2-[[4-[5-[(E)-prop-1-enyl]-2-(2H-tetrazol-5-yl)phenyl]piperazin-1-yl]methyl]benzimidazole;
1-methyl-2-[[4-[5-propyl-2-(2H-tetrazol-5-yl)phenyl]piperazin-1-yl]methyl]benzimidazole;
2-[[4-[5-isobutyl-2-(2H-tetrazol-5-yl)phenyl]piperazin-1-yl]methyl]-1-methyl-benzimidazole;
1-methyl-2-[[4-[5-(2-methylprop-1-enyl)-2-(2H-tetrazol-5-yl)phenyl]piperazin-1-yl]methyl]benzimidazole;
2-[[4-[5-methoxy-2-(2H-tetrazol-5-yl)phenyl]piperazin-1-yl]methyl]-1-methyl-benzimidazole;
2-[[4-[5-ethoxy-2-(2H-tetrazol-5-yl)phenyl]piperazin-1-yl]methyl]-1-methyl-benzimidazole;
2-[[4-[5-isopropoxy-2-(2H-tetrazol-5-yl)phenyl]piperazin-1-yl]methyl]-1-methyl-benzimidazole;
2-[[1-[5-isopropoxy-2-(2H-tetrazol-5-yl)phenyl]-4-piperidyl]methyl]-1-methyl-benzimidazole;
2-[[4-[2-(2H-tetrazol-5-yl)phenyl]piperazin-1-yl]methyl]-3H-quinazolin-4-one;
2-[[4-[5-methyl-2-(2H-tetrazol-5-yl)phenyl]piperazin-1-yl]methyl]-3H-quinazolin-4-one;
2-[[4-[5-cyclopropyl-2-(2H-tetrazol-5-yl)phenyl]piperazin-1-yl]methyl]-3H-quinazolin-4-one;
2-[[4-[2-(2H-tetrazol-5-yl)-5-(trifluoromethyl)phenyl]piperazin-1-yl]methyl]-3H-quinazolin-4-one;
2-[[4-[5-(2-methylprop-1-enyl)-2-(2H-tetrazol-5-yl)phenyl]piperazin-1-yl]methyl]-3H-quinazolin-4-one;
2-[[4-[5-isobutyl-2-(2H-tetrazol-5-yl)phenyl]piperazin-1-yl]methyl]-3H-quinazolin-4-one;
2-[[4-[5-isopropoxy-2-(2H-tetrazol-5-yl)phenyl]piperazin-1-yl]methyl]-3H-quinazolin-4-one;
2-[4-(1,3-benzothiazol-2-ylmethyl)piperazin-1-yl]-4-ethyl-N-ethylsulfonyl-benzamide;
2-[4-(1,3-benzothiazol-2-ylmethyl)piperazin-1-yl]-4-cyclopropyl-N-ethylsulfonyl-benzamide;
2-[4-(1,3-benzothiazol-2-ylmethyl)piperazin-1-yl]-N-ethylsulfonyl-4-isobutyl-benzamide;
2-[4-(1,3-benzothiazol-2-ylmethyl)piperazin-1-yl]-N-ethylsulfonyl-4-methoxy-benzamide;
2-[4-(1,3-benzothiazol-2-ylmethyl)piperazin-1-yl]-4-ethoxy-N-ethylsulfonyl-benzamide;
2-[[4-(1,3-benzothiazol-2-yl)piperazin-1-yl]methyl]-N-ethylsulfonyl-4-isopropoxy-benzamide;
2-[4-(1,3-benzothiazol-2-ylmethyl)piperazin-1-yl]-N-ethylsulfonyl-4-isopropoxy-benzamide;
2-[4-(1,3-benzothiazol-2-ylmethyl)piperazin-1-yl]-4-(cyclopropoxy)-N-ethylsulfonyl-benzamide;
2-[4-(1,3-benzothiazol-2-ylmethyl)piperazin-1-yl]-N-ethylsulfonyl-4-propoxy-benzamide;
2-[4-(1,3-benzothiazol-2-ylmethyl)piperazin-1-yl]-N-ethylsulfonyl-4-isobutoxy-benzamide;
2-[4-(1,3-benzothiazol-2-ylmethyl)piperazin-1-yl]-4-(ethylamino)-N-ethylsulfonyl-benzamide;
2-[[4-[5-ethyl-2-(2H-tetrazol-5-yl)phenyl]piperazin-1-yl]methyl]-1,3-benzothiazole;
2-[[4-[5-isopropyl-2-(2H-tetrazol-5-yl)phenyl]piperazin-1-yl]methyl]-1,3-benzothiazole;
2-[[4-[5-cyclopropyl-2-(2H-tetrazol-5-yl)phenyl]piperazin-1-yl]methyl]-1,3-benzothiazole;
2-[[4-[5-isobutyl-2-(2H-tetrazol-5-yl)phenyl]piperazin-1-yl]methyl]-1,3-benzothiazole;
2-[4-[5-isobutyl-2-(2H-tetrazol-5-yl)phenyl]piperazin-1-yl]-1,3-benzothiazole;
2-[4-[[5-isopropoxy-2-(2H-tetrazol-5-yl)phenyl]methyl]piperazin-1-yl]-1,3-benzothiazole;
2-[[4-[5-isopropoxy-2-(2H-tetrazol-5-yl)phenyl]piperazin-1-yl]methyl]-1,3-benzothiazole;
2-[[4-[5-(cyclopropoxy)-2-(2H-tetrazol-5-yl)phenyl]piperazin-1-yl]methyl]-1,3-benzothiazole;
2-[[4-[5-isobutoxy-2-(2H-tetrazol-5-yl)phenyl]piperazin-1-yl]methyl]-1,3-benzothiazole;
2-[[4-[5-(cyclopropylmethoxy)-2-(2H-tetrazol-5-yl)phenyl]piperazin-1-yl]methyl]-1,3-benzothiazole;
2-[[4-[5-(2,2-difluoroethoxy)-2-(2H-tetrazol-5-yl)phenyl]piperazin-1-yl]methyl]-1,3-benzothiazole;
2-[[4-[5-(2-methoxyethoxy)-2-(2H-tetrazol-5-yl)phenyl]piperazin-1-yl]methyl]-1,3-benzothiazole;
2-[[4-[5-(cyclopentoxy)-2-(2H-tetrazol-5-yl)phenyl]piperazin-1-yl]methyl]-1,3-benzothiazole;
2-[[4-[5-(oxetan-3-ylmethoxy)-2-(2H-tetrazol-5-yl)phenyl]piperazin-1-yl]methyl]-1,3-benzothiazole;
3-[4-(1,3-benzothiazol-2-ylmethyl)piperazin-1-yl]-N-ethyl-4-(2H-tetrazol-5-yl)aniline;
3-[4-(1,3-benzothiazol-2-ylmethyl)piperazin-1-yl]-N-isopropyl-4-(2H-tetrazol-5-yl)aniline;
3-[4-(1,3-benzothiazol-2-ylmethyl)piperazin-1-yl]-N-cyclopropyl-4-(2H-tetrazol-5-yl)aniline hydrochloride;
3-[4-(1,3-benzothiazol-2-ylmethyl)piperazin-1-yl]-N-(cyclopropylmethyl)-4-(2H-tetrazol-5-yl)aniline;
3-[4-(1,3-benzothiazol-2-ylmethyl)piperazin-1-yl]-N-(2-methoxyethyl)-4-(2H-tetrazol-5-yl)aniline;
3-[4-(1,3-benzothiazol-2-ylmethyl)piperazin-1-yl]-N-cyclopentyl-4-(2H-tetrazol-5-yl)aniline;
2-[[4-[5-(1-piperidyl)-2-(2H-tetrazol-5-yl)phenyl]piperazin-1-yl]methyl]-1,3-benzothiazole;
2-[[4-[5-(4-methylpiperazin-1-yl)-2-(2H-tetrazol-5-yl)phenyl]piperazin-1-yl]methyl]-1,3-benzothiazole;
4-[3-[4-(1,3-benzothiazol-2-ylmethyl)piperazin-1-yl]-4-(2H-tetrazol-5-yl)phenyl]morpholine;
3-[4-(1,3-benzothiazol-2-ylmethyl)piperazin-1-yl]-N-(3-methoxycyclobutyl)-4-(2H-tetrazol-5-yl)aniline;
6-[[4-[5-isobutyl-2-(2H-tetrazol-5-yl)phenyl]piperazin-1-yl]methyl]-1-methyl-7H-pyrazolo[3,4-d]pyrimidin-4-one;
2-[[4-[5-isobutyl-2-(2H-tetrazol-5-yl)phenyl]piperazin-1-yl]methyl]-7-methyl-pyrido[1,2-a]pyrimidin-4-one;
2-[[4-[5-isobutyl-2-(2H-tetrazol-5-yl)phenyl]piperazin-1-yl]methyl]oxazolo[4,5-b]pyridine;
2-[[4-[5-isobutyl-2-(2H-tetrazol-5-yl)phenyl]piperazin-1-yl]methyl]-8-methyl-pyrido[1,2-a]pyrimidin-4-one;
2-[[4-[5-isobutyl-2-(2H-tetrazol-5-yl)phenyl]piperazin-1-yl]methyl]-6-methyl-pyrido[1,2-a]pyrimidin-4-one;
5-[[4-[5-isobutyl-2-(2H-tetrazol-5-yl)phenyl]piperazin-1-yl]methyl]-1-methyl-pyrazolo[1,5-a]pyrimidin-7-one;
7-[[4-[5-isobutyl-2-(2H-tetrazol-5-yl)phenyl]piperazin-1-yl]methyl]-3-methyl-thiazolo[3,2-a]pyrimidin-5-one;
2-[[4-[5-isobutyl-2-(2H-tetrazol-5-yl)phenyl]piperazin-1-yl]methyl]pyrido[1,2-a]pyrimidin-4-one;
3-[[4-[5-isobutyl-2-(2H-tetrazol-5-yl)phenyl]piperazin-1-yl]methyl]-1-methyl-quinolin-2-one;
2-[[4-[5-isobutyl-2-(2H-tetrazol-5-yl)phenyl]piperazin-1-yl]methyl]-3-methyl-quinazolin-4-one;
3-[[4-[5-isobutyl-2-(2H-tetrazol-5-yl)phenyl]piperazin-1-yl]methyl]-1-methyl-quinoxalin-2-one;
3-[[4-[5-isobutyl-2-(2H-tetrazol-5-yl)phenyl]piperazin-1-yl]methyl]-1-methyl-indazole;
3-[[4-[5-isobutyl-2-(2H-tetrazol-5-yl)phenyl]piperazin-1-yl]methyl]-5-methyl-isoxazole;
1-[5-isobutyl-2-(2H-tetrazol-5-yl)phenyl]-4-[(1-methylimidazol-2-yl)methyl]piperazine;
1-[5-isobutyl-2-(2H-tetrazol-5-yl)phenyl]-4-[(2-methylpyrazol-3-yl)methyl]piperazine;
1-[5-isobutyl-2-(2H-tetrazol-5-yl)phenyl]-4-[[5-(trifluoromethyl)-2-pyridyl]methyl]piperazine;
3-[[4-[5-isobutyl-2-(2H-tetrazol-5-yl)phenyl]piperazin-1-yl]methyl]-5-methyl-1,2,4-oxadiazole;
4-[[4-[5-isobutyl-2-(2H-tetrazol-5-yl)phenyl]piperazin-1-yl]methyl]-2-methyl-thiazole;
2-[[4-[5-isobutyl-2-(2H-tetrazol-5-yl)phenyl]piperazin-1-yl]methyl]pyrimidine;
5-[[4-[5-isobutyl-2-(2H-tetrazol-5-yl)phenyl]piperazin-1-yl]methyl]-3-methyl-isoxazole;
3-[[4-[5-isobutyl-2-(2H-tetrazol-5-yl)phenyl]piperazin-1-yl]methyl]pyridazine;
3-[[4-[5-cyclopropyl-2-(2H-tetrazol-5-yl)phenyl]piperazin-1-yl]methyl]pyridazine;
2-[[4-[5-isobutyl-2-(2H-tetrazol-5-yl)phenyl]piperazin-1-yl]methyl]-5-methyl-1,3,4-oxadiazole;
2-[[4-[5-isobutyl-2-(2H-tetrazol-5-yl)phenyl]piperazin-1-yl]methyl]pyrazine;
1-[5-isobutyl-2-(2H-tetrazol-5-yl)phenyl]-4-[(4-methyl-1,2,4-triazol-3-yl)methyl]piperazine;
6-[[4-[5-isobutyl-2-(2H-tetrazol-5-yl)phenyl]piperazin-1-yl]methyl]-2-methyl-1H-pyrimidin-4-one;
5-[[4-[5-isobutyl-2-(2H-tetrazol-5-yl)phenyl]piperazin-1-yl]methyl]-3-methyl-1,2,4-oxadiazole;
2-[[4-[5-isobutyl-2-(2H-tetrazol-5-yl)phenyl]piperazin-1-yl]methyl]thiazole;
2-[[4-[5-isobutyl-2-(2H-tetrazol-5-yl)phenyl]piperazin-1-yl]methyl]oxazole;
4-[[4-[5-isobutyl-2-(2H-tetrazol-5-yl)phenyl]piperazin-1-yl]methyl]-2-methyl-pyrimidine;
2-[[4-[5-isobutyl-2-(2H-tetrazol-5-yl)phenyl]piperazin-1-yl]methyl]-6-methyl-1H-pyrimidin-4-one;
4-[[4-[5-isobutyl-2-(2H-tetrazol-5-yl)phenyl]piperazin-1-yl]methyl]-3,5-dimethyl-isoxazole;
5-cyclopropyl-3-[[4-[5-isobutyl-2-(2H-tetrazol-5-yl)phenyl]piperazin-1-yl]methyl]isoxazole;
5-[[4-[5-isobutyl-2-(2H-tetrazol-5-yl)phenyl]piperazin-1-yl]methyl]-2-methyl-pyrimidine;
1-[(2,5-dimethylpyrazol-3-yl)methyl]-4-[5-isobutyl-2-(2H-tetrazol-5-yl)phenyl]piperazine;
1-[5-isobutyl-2-(2H-tetrazol-5-yl)phenyl]-4-[(3-methylimidazol-4-yl)methyl]piperazine;
6-[[4-[5-isobutyl-2-(2H-tetrazol-5-yl)phenyl]piperazin-1-yl]methyl]pyridine-2-carbonitrile;
3-[[4-[5-isobutyl-2-(2H-tetrazol-5-yl)phenyl]piperazin-1-yl]methyl]-1,2,4-oxadiazole;
1-[5-isobutyl-2-(2H-tetrazol-5-yl)phenyl]-4-[(1-methylimidazol-4-yl)methyl]piperazine;
1-[5-isobutyl-2-(2H-tetrazol-5-yl)phenyl]-4-[(1-methylpyrazol-3-yl)methyl]piperazine;
4-[[4-[5-isobutyl-2-(2H-tetrazol-5-yl)phenyl]piperazin-1-yl]methyl]-5-methyl-thiazole;
3-[[4-[5-isobutyl-2-(2H-tetrazol-5-yl)phenyl]piperazin-1-yl]methyl]-5-methyl-pyridazine;
3-[[4-[5-isobutyl-2-(2H-tetrazol-5-yl)phenyl]piperazin-1-yl]methyl]-6-methyl-pyridazine;
1-[(2,3-dimethylimidazol-4-yl)methyl]-4-[5-isobutyl-2-(2H-tetrazol-5-yl)phenyl]piperazine;
3-[[4-[5-isobutyl-2-(2H-tetrazol-5-yl)phenyl]piperazin-1-yl]methyl]-6-methoxy-pyridazine;
2-[[4-[3-fluoro-5-methoxy-2-(2H-tetrazol-5-yl)phenyl]piperazin-1-yl]methyl]-1,3-benzothiazole;
2-[[4-[4-fluoro-5-methoxy-2-(2H-tetrazol-5-yl)phenyl]piperazin-1-yl]methyl]-1,3-benzothiazole;
2-[[4-[3-fluoro-5-isopropoxy-2-(2H-tetrazol-5-yl)phenyl]piperazin-1-yl]methyl]-1,3-benzothiazole;
2-[[4-[4-ethyl-5-isopropoxy-2-(2H-tetrazol-5-yl)phenyl]piperazin-1-yl]methyl]-1,3-benzothiazole;
2-[[4-[3-ethyl-5-isopropoxy-2-(2H-tetrazol-5-yl)phenyl]piperazin-1-yl]methyl]-1,3-benzothiazole;
2-[[4-[4-cyclopropyl-5-isopropoxy-2-(2H-tetrazol-5-yl)phenyl]piperazin-1-yl]methyl]-1,3-benzothiazole;
2-[[4-[4-ethoxy-5-isopropoxy-2-(2H-tetrazol-5-yl)phenyl]piperazin-1-yl]methyl]-1,3-benzothiazole;
2-[[4-[5-cyclopropyl-4-fluoro-2-(2H-tetrazol-5-yl)phenyl]piperazin-1-yl]methyl]-1,3-benzothiazole;
2-[[4-[5-cyclopropyl-3-fluoro-2-(2H-tetrazol-5-yl)phenyl]piperazin-1-yl]methyl]-1,3-benzothiazole;
2-[[4-[5-cyclopropyl-4-methyl-2-(2H-tetrazol-5-yl)phenyl]piperazin-1-yl]methyl]-1,3-benzothiazole;
2-[[4-[5-cyclopropyl-3-ethyl-2-(2H-tetrazol-5-yl)phenyl]piperazin-1-yl]methyl]-1,3-benzothiazole;
4-[4-(1,3-benzothiazol-2-ylmethyl)piperazin-1-yl]-2-cyclopropyl-N-ethyl-5-(2H-tetrazol-5-yl)aniline;
N-[4-[4-(1,3-benzothiazol-2-ylmethyl)piperazin-1-yl]-2-cyclopropyl-5-(2H-tetrazol-5-yl)phenyl]acetamide;
2-[[4-[4-fluoro-5-isobutyl-2-(2H-tetrazol-5-yl)phenyl]piperazin-1-yl]methyl]-1,3-benzothiazole;
2-[[4-[5-isobutyl-4-methyl-2-(2H-tetrazol-5-yl)phenyl]piperazin-1-yl]methyl]-1,3-benzothiazole;
4-[4-(1,3-benzothiazol-2-ylmethyl)piperazin-1-yl]-2-isobutyl-5-(2H-tetrazol-5-yl)aniline;
4-[4-(1,3-benzothiazol-2-ylmethyl)piperazin-1-yl]-2-isobutyl-N-methyl-5-(2H-tetrazol-5-yl)aniline;
4-[4-(1,3-benzothiazol-2-ylmethyl)piperazin-1-yl]-N-ethyl-2-isobutyl-5-(2H-tetrazol-5-yl)aniline;
2-[[4-[5-ethyl-2-(2H-tetrazol-5-yl)-3-pyridyl]piperazin-1-yl]methyl]-1,3-benzothiazole;
2-[[4-[6-ethyl-3-(2H-tetrazol-5-yl)-2-pyridyl]piperazin-1-yl]methyl]-1,3-benzothiazole;
2-[[4-[5-cyclopropyl-2-(2H-tetrazol-5-yl)-3-pyridyl]piperazin-1-yl]methyl]-1,3-benzothiazole;
2-[[4-[2-cyclopropyl-5-(2H-tetrazol-5-yl)-4-pyridyl]piperazin-1-yl]methyl]-1,3-benzothiazole;
2-[[4-[6-cyclopropyl-3-(2H-tetrazol-5-yl)-2-pyridyl]piperazin-1-yl]methyl]-1,3-benzothiazole;
2-[[4-[6-cyclopropyl-3-(2H-tetrazol-5-yl)pyrazin-2-yl]piperazin-1-yl]methyl]-1,3-benzothiazole;
2-[[4-[5-isobutyl-2-(2H-tetrazol-5-yl)-3-pyridyl]piperazin-1-yl]methyl]-1,3-benzothiazole;
2-[[4-[6-isobutyl-3-(2H-tetrazol-5-yl)-2-pyridyl]piperazin-1-yl]methyl]-1,3-benzothiazole;
5-[4-(1,3-benzothiazol-2-ylmethyl)piperazin-1-yl]-N-(cyclopropylmethyl)-6-(2H-tetrazol-5-yl)pyridin-3-amine;
2-[[4-[3-fluoro-5-methoxy-2-(2H-tetrazol-5-yl)phenyl]piperazin-1-yl]methyl]-3H-quinazolin-4-one;
2-[[4-[3-fluoro-5-isopropoxy-2-(2H-tetrazol-5-yl)phenyl]piperazin-1-yl]methyl]-3H-quinazolin-4-one;
2-[[4-[3-cyclopropyl-5-isopropoxy-2-(2H-tetrazol-5-yl)phenyl]piperazin-1-yl]methyl]-3H-quinazolin-4-one;
2-[[4-[4-fluoro-5-isobutyl-2-(2H-tetrazol-5-yl)phenyl]piperazin-1-yl]methyl]-3H-quinazolin-4-one;
3-[[4-[3-fluoro-5-methoxy-2-(2H-tetrazol-5-yl)phenyl]piperazin-1-yl]methyl]pyridazine;
3-[[4-[3-fluoro-5-isopropoxy-2-(2H-tetrazol-5-yl)phenyl]piperazin-1-yl]methyl]pyridazine;
3-[[4-[2-fluoro-3-isopropoxy-6-(2H-tetrazol-5-yl)phenyl]piperazin-1-yl]methyl]pyridazine;
3-[[4-[4-ethyl-5-isopropoxy-2-(2H-tetrazol-5-yl)phenyl]piperazin-1-yl]methyl]pyridazine;
3-[[4-[3-ethyl-5-isopropoxy-2-(2H-tetrazol-5-yl)phenyl]piperazin-1-yl]methyl]pyridazine;
3-[[4-[4-cyclopropyl-5-isopropoxy-2-(2H-tetrazol-5-yl)phenyl]piperazin-1-yl]methyl]pyridazine;
3-[[4-[3-cyclopropyl-5-isopropoxy-2-(2H-tetrazol-5-yl)phenyl]piperazin-1-yl]methyl]pyridazine;
3-[[4-[4-ethoxy-5-isopropoxy-2-(2H-tetrazol-5-yl)phenyl]piperazin-1-yl]methyl]pyridazine;
3-[[4-[4-fluoro-5-isobutyl-2-(2H-tetrazol-5-yl)phenyl]piperazin-1-yl]methyl]pyridazine;
3-[[4-[3-fluoro-5-isobutyl-2-(2H-tetrazol-5-yl)phenyl]piperazin-1-yl]methyl]pyridazine;
3-[[4-[2-fluoro-3-isobutyl-6-(2H-tetrazol-5-yl)phenyl]piperazin-1-yl]methyl]pyridazine;
3-[[4-[5-isobutyl-4-methyl-2-(2H-tetrazol-5-yl)phenyl]piperazin-1-yl]methyl]pyridazine;
2-isobutyl-N-methyl-4-[4-(pyridazin-3-ylmethyl)piperazin-1-yl]-5-(2H-tetrazol-5-yl)aniline;
N-ethyl-2-isobutyl-4-[4-(pyridazin-3-ylmethyl)piperazin-1-yl]-5-(2H-tetrazol-5-yl)aniline;
3-[[(2S)-4-[5-isobutyl-2-(2H-tetrazol-5-yl)-3-pyridyl]-2-methyl-piperazin-1-yl]methyl]pyridazine;
3-[[4-[3-fluoro-5-isobutyl-2-(2H-tetrazol-5-yl)phenyl]-2-isopropyl-piperazin-1-yl]methyl]pyridazine;
2-[3-fluoro-5-isobutyl-2-(2H-tetrazol-5-yl)phenyl]-5-(pyridazin-3-ylmethyl)-2,5-diazabicyclo[2.2.1]heptane;
3-[3-fluoro-5-isobutyl-2-(2H-tetrazol-5-yl)phenyl]-8-(pyridazin-3-ylmethyl)-3, 8-diazabicyclo[3.2.1]octane;
1-[(5-chloro-2-pyridyl)methyl]-4-[3-fluoro-5-isobutyl-2-(2H-tetrazol-5-yl)phenyl]piperazine;
1-[3-fluoro-5-isobutyl-2-(2H-tetrazol-5-yl)phenyl]-4-[(5-methoxy-2-pyridyl)methyl]piperazine;
1-[3-fluoro-5-isobutyl-2-(2H-tetrazol-5-yl)phenyl]-4-[(5-fluoro-2-pyridyl)methyl]piperazine;
1-[3-fluoro-5-isobutyl-2-(2H-tetrazol-5-yl)phenyl]-4-[(3-fluoro-2-pyridyl)methyl]piperazine;
1-[3-fluoro-5-isobutyl-2-(2H-tetrazol-5-yl)phenyl]-4-[(3-methoxy-2-pyridyl)methyl]piperazine;
1-[3-fluoro-5-isobutyl-2-(2H-tetrazol-5-yl)phenyl]-4-[(4-methoxy-3,5-dimethyl-2-pyridyl)methyl]piperazine;
1-[3-fluoro-5-isobutyl-2-(2H-tetrazol-5-yl)phenyl]-4-[(5-methyl-2-pyridyl)methyl]piperazine;
1-[3-fluoro-5-isobutyl-2-(2H-tetrazol-5-yl)phenyl]-4-[[4-(trifluoromethyl)-2-pyridyl]methyl]piperazine;
1-[(3-chloro-5-fluoro-2-pyridyl)methyl]-4-[3-fluoro-5-isobutyl-2-(2H-tetrazol-5-yl)phenyl]piperazine;
1-[(4,5-dimethoxy-2-pyridyl)methyl]-4-[3-fluoro-5-isobutyl-2-(2H-tetrazol-5-yl)phenyl]piperazine;
1-[3-fluoro-5-isobutyl-2-(2H-tetrazol-5-yl)phenyl]-4-[(4-methoxy-3-methyl-2-pyridyl)methyl]piperazine;
6-[[4-[3-fluoro-5-isobutyl-2-(2H-tetrazol-5-yl)phenyl]piperazin-1-yl]methyl]pyridine-3-carbonitrile;
1-[(4-chloro-5-fluoro-2-pyridyl)methyl]-4-[3-fluoro-5-isobutyl-2-(2H-tetrazol-5-yl)phenyl]piperazine;
1-[(3,5-difluoro-2-pyridyl)methyl]-4-[3-fluoro-5-isobutyl-2-(2H-tetrazol-5-yl)phenyl]piperazine;
1-[3-fluoro-5-isobutyl-2-(2H-tetrazol-5-yl)phenyl]-4-[1-(2-pyridyl)ethyl]piperazine;
[(2S)-4-[3-fluoro-5-isobutyl-2-(2H-tetrazol-5-yl)phenyl]-2-methyl-piperazin-1-yl]-(2-pyridyl)methanone;
2-[[4-[2-fluoro-3-isobutyl-6-(2H-tetrazol-5-yl)phenyl]piperazin-1-yl]methyl]-5-methyl-thiazole;
2-[[4-[5-isobutyl-2-(2H-tetrazol-5-yl)-3-pyridyl]piperazin-1-yl]methyl]-5-methyl-thiazole;
1-[5-isobutyl-2-(2H-tetrazol-5-yl)phenyl]-4-(pyridazin-3-ylmethyl)-1,4-diazepane;
5-[5-isobutyl-2-(2H-tetrazol-5-yl)phenyl]-2-(pyridazin-3-ylmethyl)-1,3,3a,4,6,6a-hexahydropyrrolo[3,4-c]pyrrole;
5-[3-fluoro-5-isobutyl-2-(2H-tetrazol-5-yl)phenyl]-2-(pyridazin-3-ylmethyl)-1,3,3a,4,6,6a-hexahydropyrrolo[3,4-c]pyrrole;
3-[[4-[5-isobutyl-2-(2H-tetrazol-5-yl)phenyl]-1-piperidyl]methyl]pyridazine;
3-[[3-[5-isobutyl-2-(2H-tetrazol-5-yl)phenyl]pyrrolidin-1-yl]methyl]pyridazine;
1-(5-isobutyl-2-(2H-tetrazol-5-yl)phenyl)-N-(pyridazin-3-ylmethyl)azetidin-3-amine;
2-((1-(3-fluoro-5-isobutyl-2-(2H-tetrazol-5-yl)phenyl)piperidin-4-yl)methyl)pyridine;
3-[[4-[[5-isobutyl-2-(2H-tetrazol-5-yl)phenyl]methyl]piperazin-1-yl]methyl]pyridazine;
(5-isobutyl-2-(2H-tetrazol-5-yl)phenyl)(4-(pyridazin-3-ylmethyl)piperazin-1-yl)methanone;
2-ethyl-3-[[1-[2-(2H-tetrazol-5-yl)phenyl]-4-piperidyl]methyl]quinazolin-4-one;
2-[4-[(2-ethyl-4-oxo-quinazolin-3-yl)methyl]-1-piperidyl]-N-ethylsulfonyl-benzamide;
2-ethyl-6-nitro-3-[[1-[2-(2H-tetrazol-5-yl)phenyl]-4-piperidyl]methyl]quinazolin-4-one;
N-[2-ethyl-4-oxo-3-[[1-[2-(2H-tetrazol-5-yl)phenyl]-4-piperidyl]methyl]quinazolin-6-yl]-N-propyl-thiophene-2-carboxamide;
N-[2-ethyl-4-oxo-3-[[1-[2-(2H-tetrazol-5-yl)phenyl]-4-piperidyl]methyl]quinazolin-6-yl]-N-(3-pyridylmethyl)thiophene-2-carboxamide;
N-[2-ethyl-4-oxo-3-[[1-[2-(2H-tetrazol-5-yl)phenyl]-4-piperidyl]methyl]quinazolin-6-yl]benzamide;
N-benzyl-N-[2-ethyl-4-oxo-3-[[1-[2-(2H-tetrazol-5-yl)phenyl]-4-piperidyl]methyl]quinazolin-6-yl]thiophene-2-carboxamide;
N-[2-ethyl-4-oxo-3-[[1-[2-(2H-tetrazol-5-yl)phenyl]-4-piperidyl]methyl]quinazolin-6-yl]cyclopropanecarboxamide;
N-[(2-chlorophenyl)methyl]-N-[2-ethyl-4-oxo-3-[[1-[2-(2H-tetrazol-5-yl)phenyl]-4-piperidyl]methyl]quinazolin-6-yl]thiophene-2-carboxamide;
2-phenyl-3-[[1-[2-(1H-tetrazol-5-yl)phenyl]-4-piperidyl]methyl]quinazolin-4-one;
3-[[(2S)-4-[5-(1,2-dideuterio-2-methyl-propyl)-3-f(luoro-2-(2H-tetrazol-5-yl)phenyl]-2-methyl-piperazin-1-yl]methyl]pyridazine; and
2,2,3,3,5,6,6-heptadeuterio-1-[3-fluoro-5-isobutyl-2-(2H-tetrazol-5-yl)phenyl]-4-(pyridazin-3-ylmethyl)-5-(trideuteriomethyl)piperazine;
or a pharmaceutically acceptable salt thereof.

18. A pharmaceutical composition comprising the compound of claim 6, and optionally a pharmaceutically acceptable carrier.

19. A method of treating or preventing chronic pain, comprising administering to a subject in need thereof the compound of claim 6.

20. The method of claim 19, wherein the pain is diabetic peripheral neuropathic pain.
